SEQUENCE LISTING (612 sequences)

SEQ ID NO: 1 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 2 - DNA, 206 bp, synthetic construct
  source 1..206: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaa

SEQ ID NO: 3 - DNA, 91 bp, synthetic construct
  source 1..91: mol_type = other DNA
aaaaaaaaagctgccatcagtcggcgtggactgtagaacactgccaatgccggtcccaagcccggataaaagtggagggtacagtccacgc

SEQ ID NO: 4 - DNA, 373 bp, synthetic construct
  source 1..373: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaagttttagagctagaaatagcaagttaaaataaggctagtccgttatcaacttgaaaaagtggcaccgagtcggtgcaaaaaaaaagctgccatcagtcggcgtggactgtagaacactgccaatgccggtcccaagcccggataaaagtggagggtacagtccacgc

SEQ ID NO: 5 - DNA, 155 bp, synthetic construct
  source 1..155: mol_type = other DNA
cggcagcatagcaagttaaaataaggctagtccgttatcaacttgaaaaagtggcaccgagtcggtgcaaaaaaaaagctgccatcagtcggcgtggactgtagaaccatgccgactgatggcagaaaaaaaaaagttttagagctatgctgccg

SEQ ID NO: 6 - DNA, 20 bp, synthetic construct
  source 1..20: mol_type = other DNA
gtcgtcactagtgtcggcta

SEQ ID NO: 7 - DNA, 27 bp, synthetic construct
  source 1..27: mol_type = other DNA
gcctgttccgtggccgacactagtgac

SEQ ID NO: 8 - DNA, 64 bp, synthetic construct
  source 1..64: mol_type = other DNA
aaggaattgggtcaggagccccccccctgaacccaggataaccctcaaagtcggggggcaaccc

SEQ ID NO: 9 - DNA, 45 bp, synthetic construct
  source 1..45: mol_type = other DNA
taaaggaacgcggttctatctagttacgcgttaaaccaactagaa

SEQ ID NO: 10 - DNA, 20 bp, synthetic construct
  source 1..20: mol_type = other DNA
ctggcaaactgcctgttccg

SEQ ID NO: 11 - DNA, 20 bp, synthetic construct
  source 1..20: mol_type = other DNA
ctagtgacgacgctctgcta

SEQ ID NO: 12 - DNA, 20 bp, synthetic construct
  source 1..20: mol_type = other DNA
ccagtgcttttcaagatacc

SEQ ID NO: 13 - DNA, 20 bp, synthetic construct
  source 1..20: mol_type = other DNA
ggcccagactgagcacgtga

SEQ ID NO: 14 - DNA, 23 bp, synthetic construct
  source 1..23: mol_type = other DNA
tctgccatctcgtgctcagtctg

SEQ ID NO: 15 - DNA, 20 bp, synthetic construct
  source 1..20: mol_type = other DNA
ggcccagactgagcacgtga

SEQ ID NO: 16 - DNA, 23 bp, synthetic construct
  source 1..23: mol_type = other DNA
tctgccatcgcgtgctcagtctg

SEQ ID NO: 17 - DNA, 23 bp, synthetic construct
  source 1..23: mol_type = other DNA
tctgccatcccgtgctcagtctg

SEQ ID NO: 18 - DNA, 23 bp, synthetic construct
  source 1..23: mol_type = other DNA
tctgccattacgtgctcagtctg

SEQ ID NO: 19 - DNA, 23 bp, synthetic construct
  source 1..23: mol_type = other DNA
tctgccatgacgtgctcagtctg

SEQ ID NO: 20 - DNA, 23 bp, synthetic construct
  source 1..23: mol_type = other DNA
tctgccataacgtgctcagtctg

SEQ ID NO: 21 - DNA, 23 bp, synthetic construct
  source 1..23: mol_type = other DNA
tctgccagcacgtgctcagtctg

SEQ ID NO: 22 - DNA, 23 bp, synthetic construct
  source 1..23: mol_type = other DNA
tctgccaccacgtgctcagtctg

SEQ ID NO: 23 - DNA, 23 bp, synthetic construct
  source 1..23: mol_type = other DNA
tctgccaacacgtgctcagtctg

SEQ ID NO: 24 - DNA, 23 bp, synthetic construct
  source 1..23: mol_type = other DNA
tctgccttcacgtgctcagtctg

SEQ ID NO: 25 - DNA, 23 bp, synthetic construct
  source 1..23: mol_type = other DNA
tctgccgtcacgtgctcagtctg

SEQ ID NO: 26 - DNA, 23 bp, synthetic construct
  source 1..23: mol_type = other DNA
tctgccctcacgtgctcagtctg

SEQ ID NO: 27 - DNA, 23 bp, synthetic construct
  source 1..23: mol_type = other DNA
tctgctatcacgtgctcagtctg

SEQ ID NO: 28 - DNA, 23 bp, synthetic construct
  source 1..23: mol_type = other DNA
tctgcgatcacgtgctcagtctg

SEQ ID NO: 29 - DNA, 23 bp, synthetic construct
  source 1..23: mol_type = other DNA
tctgcaatcacgtgctcagtctg

SEQ ID NO: 30 - DNA, 23 bp, synthetic construct
  source 1..23: mol_type = other DNA
tctgtcatcacgtgctcagtctg

SEQ ID NO: 31 - DNA, 23 bp, synthetic construct
  source 1..23: mol_type = other DNA
tctggcatcacgtgctcagtctg

SEQ ID NO: 32 - DNA, 23 bp, synthetic construct
  source 1..23: mol_type = other DNA
tctgacatcacgtgctcagtctg

SEQ ID NO: 33 - DNA, 23 bp, synthetic construct
  source 1..23: mol_type = other DNA
tcttccatcacgtgctcagtctg

SEQ ID NO: 34 - DNA, 23 bp, synthetic construct
  source 1..23: mol_type = other DNA
tctcccatcacgtgctcagtctg

SEQ ID NO: 35 - DNA, 23 bp, synthetic construct
  source 1..23: mol_type = other DNA
tctaccatcacgtgctcagtctg

SEQ ID NO: 36 - DNA, 23 bp, synthetic construct
  source 1..23: mol_type = other DNA
tcggccatcacgtgctcagtctg

SEQ ID NO: 37 - DNA, 23 bp, synthetic construct
  source 1..23: mol_type = other DNA
tccgccatcacgtgctcagtctg

SEQ ID NO: 38 - DNA, 23 bp, synthetic construct
  source 1..23: mol_type = other DNA
tcagccatcacgtgctcagtctg

SEQ ID NO: 39 - DNA, 47 bp, synthetic construct
  source 1..47: mol_type = other DNA
tggaggaagcagggcttcctttcctctgccatctcgtgctcagtctg

SEQ ID NO: 40 - DNA, 47 bp, synthetic construct
  source 1..47: mol_type = other DNA
tggaggaagcagggcttcctttgctctgccatcacgtgctcagtctg

SEQ ID NO: 41 - DNA, 47 bp, synthetic construct
  source 1..47: mol_type = other DNA
tggaggaagcagggcttcctatcctctgccatcacgtgctcagtctg

SEQ ID NO: 42 - DNA, 20 bp, synthetic construct
  source 1..20: mol_type = other DNA
gattcctggtgccagaaaca

SEQ ID NO: 43 - DNA, 60 bp, synthetic construct
  source 1..60: mol_type = other DNA
tctgccctcccgtcacccctgtcttatcgtcgtcatccttgtaatcttctggcaccagga

SEQ ID NO: 44 - DNA, 50 bp, synthetic construct
  source 1..50: mol_type = other DNA
cctcttctttgacgcggttctatctagttacgcgttaaaccaactagaaa

SEQ ID NO: 45 - DNA, 64 bp, synthetic construct
  source 1..64: mol_type = other DNA
cctcttctgggtcaggagccccccccctgaacccaggataaccctcaaagtcggggggcaaccc

SEQ ID NO: 46 - DNA, 20 bp, synthetic construct
  source 1..20: mol_type = other DNA
gcattttcaggaggaagcga

SEQ ID NO: 47 - DNA, 54 bp, synthetic construct
  source 1..54: mol_type = other DNA
tgtctgaagccatcccttatcgtcgtcatccttgtaatccttcctcctgaaaat

SEQ ID NO: 48 - DNA, 50 bp, synthetic construct
  source 1..50: mol_type = other DNA
aactctctttgacgcggttctatctagttacgcgttaaaccaactagaaa

SEQ ID NO: 49 - DNA, 64 bp, synthetic construct
  source 1..64: mol_type = other DNA
aactctctgggtcaggagccccccccctgaacccaggataaccctcaaagtcggggggcaaccc

SEQ ID NO: 50 - DNA, 20 bp, synthetic construct
  source 1..20: mol_type = other DNA
gatgtctgcaggccagatga

SEQ ID NO: 51 - DNA, 59 bp, synthetic construct
  source 1..59: mol_type = other DNA
aatgtgccatctggagcactcacttatcgtcgtcatccttgtaatctctggcctgcaga

SEQ ID NO: 52 - DNA, 50 bp, synthetic construct
  source 1..50: mol_type = other DNA
acaatctcttgacgcggttctatctagttacgcgttaaaccaactagaaa

SEQ ID NO: 53 - DNA, 68 bp, synthetic construct
  source 1..68: mol_type = other DNA
acaatctcgtcagggtcaggagccccccccctgcacccaggaaaaccctcaaagtcggggggcaaccc

SEQ ID NO: 54 - DNA, 34 bp, synthetic construct
  source 1..34: mol_type = other DNA
ccttattttaacttgctatttctagctctaaaac

SEQ ID NO: 55 - DNA, 23 bp, synthetic construct
  source 1..23: mol_type = other DNA
atcaacttgaaaaagtggcaccg

SEQ ID NO: 56 - DNA, 23 bp, synthetic construct
  source 1..23: mol_type = other DNA
cggtgccactttttcaagttgat

SEQ ID NO: 57 - DNA, 34 bp, synthetic construct
  source 1..34: mol_type = other DNA
gttttagagctagaaatagcaagttaaaataagg

SEQ ID NO: 58 - DNA, 53 bp, synthetic construct
  source 1..53: mol_type = other DNA
acactctttccctacacgacgctcttccgatctatgtgggctgcctagaaagg

SEQ ID NO: 59 - DNA, 54 bp, synthetic construct
  source 1..54: mol_type = other DNA
gtgactggagttcagacgtgtgctcttccgatctcccagccaaacttgtcaacc

SEQ ID NO: 60 - DNA, 54 bp, synthetic construct
  source 1..54: mol_type = other DNA
acactctttccctacacgacgctcttccgatctcacaacagcttcatgtcagcc

SEQ ID NO: 61 - DNA, 52 bp, synthetic construct
  source 1..52: mol_type = other DNA
tggagttcagacgtgtgctcttccgatctacgttaatgtttcctgatggtcc

SEQ ID NO: 62 - DNA, 54 bp, synthetic construct
  source 1..54: mol_type = other DNA
acactctttccctacacgacgctcttccgatctacaaacaagacagggaactgg

SEQ ID NO: 63 - DNA, 50 bp, synthetic construct
  source 1..50: mol_type = other DNA
tggagttcagacgtgtgctcttccgatctctagaggggtgaggctgaaac

SEQ ID NO: 64 - DNA, 55 bp, synthetic construct
  source 1..55: mol_type = other DNA
acactctttccctacacgacgctcttccgatctacttggtgccaaattcttctcc

SEQ ID NO: 65 - DNA, 50 bp, synthetic construct
  source 1..50: mol_type = other DNA
tggagttcagacgtgtgctcttccgatctaaagagggaatgggctttgga

SEQ ID NO: 66 - DNA, 1903 bp, synthetic construct
  source 1..1903: mol_type = other DNA
tctggctaactagagaacccactgcttactggcttatcgaaattaatacgactcactatagggagacccaagctggctagcgtttaaacgggccctctagactcgaggccaccatggtgagcaagggcgaggaggacaacatggccatcattaaggagttcatgaggttcaaggtccacatggagggaagcgtgaacggccacgagttcgagatcgagggagagggcgagggcagaccttacgagggcacccagaccgccaagctgaaggtcaccaagggaggccccctcccctttgcttgggacatcctgtccccccagttcatgtacggcagcaaggcctatgtcaagcaccccgccgatatccccgactacctgaagctgtccttccccgagggcttcaagtgggagagggtgatgaacttcgaggacggcggcgtggtcaccgtgacacaggactccagcctccaggacggcgagttcatctacaaggtgaagctgaggggcaccaacttccctagcgacggccctgtcatgcagaagaagaccatgggatgggaggccagctccgagaggatgtaccccgaggacggcgctctgaagggcgagatcaagcagaggctcaagctgaaggacggcggccactacgacgccgaggtgaagaccacctacaaagccaagaagcccgtgcaactgcccggcgcctacaacgtcaacatcaagctcgacatcacctcccacaacgaggactacaccatcgtggagcaatacgagagggccgagggaagacacagcacaggcggcatggacgagctgtacaagggctccggcgccaccaacttcagcctgctcaaacaagccggcgatgtggaggagaaccctggacccgactacaaggacgacgacgacaaggtgagcaaaggcgaggagctgttcaccggcgtggtgcctatcctggtcgagctggacggcgatgtgaacggccacaagttcagcgtgtccggcgaaggagagggcgatgccacctatggcaagctgaccctgaagtttatttgcactactggcaaactgcctgttccgtagccgacactagtgacgacgctctgctatggcgtccagtgcttttcaagatacccggatcacatgaaacggcatgactttttcaagagtgccatgcccgaaggttatgtacaggaaaggaccatctttttcaaagacgacggcaactacaagaccagggctgaggtgaagttcgagggcgataccctcgtgaacagaatcgagctcaagggcatcgacttcaaggaggacggcaacatcctcggccacaagctggaatacaattacaactcccacaacgtctatatcatggccgataagcagaagaacggcattaaggtcaatttcaagatcaggcacaacatcgaggacggcagcgtgcagctggccgatcactaccagcagaacacccccatcggagacggacctgtgctgctgcctgacaaccattatctgagcacccagtccgccctgtccaaggaccctaacgagaagagggaccacatggtcctcctggagttcgtgaccgccgctggcatcaccctgggaatggacgagctctacaaatgaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 67 - DNA, 3404 bp, synthetic construct
  source 1..3404: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtggcaccgagtcggtgctctgccattacgtgctcagtctgaaaaaaaaagctgccatcagtcggcgtggactgtagaacactgccaatgccggtcccaagcccggataaaagtggagggtacagtccacgctttttttcatggtcatagctgtttcctttttttcgtactgagtcgcccagtctcagaatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 68 - DNA, 350 bp, unidentified
  source 1..350: mol_type = unassigned DNA
ggccaaagcatgcatggggtcgtagcgaactgagagtttcagagctatgctggaaacagcatagcaagttgaaataaggctagtccgttatcaacttgaaaaagtggcaccgagtcggtgccctcggcccttctcagttcgctacgaaaaaaaaaagctgccatcagtcggcgtggactgtagaacactgccaatgccggtcccaagcccggataaaagtggagggtacagtccacgctttttttggttttggtcttaggaactttgctgccacaataccttggcccttctcagttcgctacgacccatacacccaaaggtccgctaagcagcttggcgtaactagatct

SEQ ID NO: 69 - DNA, 350 bp, unidentified
  source 1..350: mol_type = unassigned DNA
ggccaaagcatgcatggggtcgtagcgaactgagagtttcagagctatgctggaaacagcatagcaagttgaaataaggctagtccgttatcaacttgaaaaagtggcaccgagtcggtgcaatacctcggcccttctcagttcgctacgaaaaaaaaaagctgccatcagtcggcgtggactgtagaacactgccaatgccggtcccaagcccggataaaagtggagggtacagtccacgctttttttttggtcttaggaactttgctgccacaataccttggcccttctcagttcgctacgacccatacacccaacaggcagctaagcagcttggcgtaactagatct

SEQ ID NO: 70 - DNA, 350 bp, unidentified
  source 1..350: mol_type = unassigned DNA
ggccaaagcatgcatggggtcgtagcgaactgagagtttcagagctatgctggaaacagcatagcaagttgaaataaggctagtccgttatcaacttgaaaaagtggcaccgagtcggtgcgctgccacaatacctcggcccttctcagttcgctacgaaaaaaaaaagctgccatcagtcggcgtggactgtagaacactgccaatgccggtcccaagcccggataaaagtggagggtacagtccacgctttttttaggaactttgctgccacaataccttggcccttctcagttcgctacgacccatacacccaagaaggtgctaagcagcttggcgtaactagatct

SEQ ID NO: 71 - DNA, 350 bp, unidentified
  source 1..350: mol_type = unassigned DNA
ggccaaagcatgcatgggtcgtagcgaactgagaagtttcagagctatgctggaaacagcatagcaagttgaaataaggctagtccgttatcaacttgaaaaagtggcaccgagtcggtgccctcggcccttctcagttcgctacgaaaaaaaaagctgccatcagtcggcgtggactgtagaacactgccaatgccggtcccaagcccggataaaagtggagggtacagtccacgctttttttgtggttttggtcttaggaactttgctgccacaataccttggcccttctcagttcgctacgacccatacacccagcattggctaagcagcttggcgtaactagatct

SEQ ID NO: 72 - DNA, 350 bp, unidentified
  source 1..350: mol_type = unassigned DNA
ggccaaagcatgcatgggtcgtagcgaactgagaagtttcagagctatgctggaaacagcatagcaagttgaaataaggctagtccgttatcaacttgaaaaagtggcaccgagtcggtgcaatacctcggcccttctcagttcgctacgaaaaaaaaagctgccatcagtcggcgtggactgtagaacactgccaatgccggtcccaagcccggataaaagtggagggtacagtccacgctttttttttttggtcttaggaactttgctgccacaataccttggcccttctcagttcgctacgacccatacacccacttaacgctaagcagcttggcgtaactagatct

SEQ ID NO: 73 - DNA, 350 bp, unidentified
  source 1..350: mol_type = unassigned DNA
ggccaaagcatgcatgggtcgtagcgaactgagaagtttcagagctatgctggaaacagcatagcaagttgaaataaggctagtccgttatcaacttgaaaaagtggcaccgagtcggtgccacaatacctcggcccttctcagttcgctacgaaaaaaaaagctgccatcagtcggcgtggactgtagaacactgccaatgccggtcccaagcccggataaaagtggagggtacagtccacgcttttttttggtcttaggaactttgctgccacaataccttggcccttctcagttcgctacgacccatacacccagcgtaagctaagcagcttggcgtaactagatct

SEQ ID NO: 74 - DNA, 350 bp, unidentified
  source 1..350: mol_type = unassigned DNA
ggccaaagcatgcatgggtcgtagcgaactgagaagtttcagagctatgctggaaacagcatagcaagttgaaataaggctagtccgttatcaacttgaaaaagtggcaccgagtcggtgcgctgccacaatacctcggcccttctcagttcgctacgaaaaaaaaagctgccatcagtcggcgtggactgtagaacactgccaatgccggtcccaagcccggataaaagtggagggtacagtccacgctttttttttaggaactttgctgccacaataccttggcccttctcagttcgctacgacccatacacccattctaagctaagcagcttggcgtaactagatct

SEQ ID NO: 75 - DNA, 350 bp, unidentified
  source 1..350: mol_type = unassigned DNA
ggccaaagcatgcatgagcgaactgagaagggccagtttcagagctatgctggaaacagcatagcaagttgaaataaggctagtccgttatcaacttgaaaaagtggcaccgagtcggtgccctcggcccttctcagttcaaaaaaaaagctgccatcagtcggcgtggactgtagaacactgccaatgccggtcccaagcccggataaaagtggagggtacagtccacgcttttttttcactcaagcctgtggttttggtcttaggaactttgctgccacaataccttggcccttctcagttcgctacgacccataactagcgctaagcagcttggcgtaactagatct

SEQ ID NO: 76 - DNA, 350 bp, unidentified
  source 1..350: mol_type = unassigned DNA
ggccaaagcatgcatgagcgaactgagaagggccagtttcagagctatgctggaaacagcatagcaagttgaaataaggctagtccgttatcaacttgaaaaagtggcaccgagtcggtgcaatacctcggcccttctcagttcaaaaaaaaagctgccatcagtcggcgtggactgtagaacactgccaatgccggtcccaagcccggataaaagtggagggtacagtccacgcttttttttcaagcctgtggttttggtcttaggaactttgctgccacaataccttggcccttctcagttcgctacgacccatagtaagggctaagcagcttggcgtaactagatct

SEQ ID NO: 77 - DNA, 350 bp, unidentified
  source 1..350: mol_type = unassigned DNA
ggccaaagcatgcatgagcgaactgagaagggccagtttcagagctatgctggaaacagcatagcaagttgaaataaggctagtccgttatcaacttgaaaaagtggcaccgagtcggtgccacaatacctcggcccttctcagttcaaaaaaaaagctgccatcagtcggcgtggactgtagaacactgccaatgccggtcccaagcccggataaaagtggagggtacagtccacgctttttttagcctgtggttttggtcttaggaactttgctgccacaataccttggcccttctcagttcgctacgacccatatacagcgctaagcagcttggcgtaactagatct

SEQ ID NO: 78 - DNA, 350 bp, unidentified
  source 1..350: mol_type = unassigned DNA
ggccaaagcatgcatgagcgaactgagaagggccagtttcagagctatgctggaaacagcatagcaagttgaaataaggctagtccgttatcaacttgaaaaagtggcaccgagtcggtgcgctgccacaatacctcggcccttctcagttcaaaaaaaaagctgccatcagtcggcgtggactgtagaacactgccaatgccggtcccaagcccggataaaagtggagggtacagtccacgctttttttgtggttttggtcttaggaactttgctgccacaataccttggcccttctcagttcgctacgacccataccccgagctaagcagcttggcgtaactagatct

SEQ ID NO: 79 - DNA, 350 bp, unidentified
  source 1..350: mol_type = unassigned DNA
ggccaaagcatgcatgcctcaatcctttgggtgtagtttcagagctatgctggaaacagcatagcaagttgaaataaggctagtccgttatcaacttgaaaaagtggcaccgagtcggtgcgctacgacccatacacccaaaggattgaaaaaaaaagctgccatcagtcggcgtggactgtagaacactgccaatgccggtcccaagcccggataaaagtggagggtacagtccacgctttttttgctgccacaatacctcggcccttctcagttcgctgcgacccatacacccaaaggattgaggtcttggacaactgcaggctaagcagcttggcgtaactagatct

SEQ ID NO: 80 - DNA, 350 bp, unidentified
  source 1..350: mol_type = unassigned DNA
ggccaaagcatgcatgcctcaatcctttgggtgtagtttcagagctatgctggaaacagcatagcaagttgaaataaggctagtccgttatcaacttgaaaaagtggcaccgagtcggtgcgttcgctacgacccatacacccaaaggattgaaaaaaaaagctgccatcagtcggcgtggactgtagaacactgccaatgccggtcccaagcccggataaaagtggagggtacagtccacgctttttttccacaatacctcggcccttctcagttcgctgcgacccatacacccaaaggattgaggtcttggacaactgctggctaagcagcttggcgtaactagatct

SEQ ID NO: 81 - DNA, 350 bp, unidentified
  source 1..350: mol_type = unassigned DNA
ggccaaagcatgcatgcctcaatcctttgggtgtagtttcagagctatgctggaaacagcatagcaagttgaaataaggctagtccgttatcaacttgaaaaagtggcaccgagtcggtgcccttctcagttcgctacgacccatacacccaaaggattgaaaaaaaaagctgccatcagtcggcgtggactgtagaacactgccaatgccggtcccaagcccggataaaagtggagggtacagtccacgctttttttcctcggcccttctcagttcgctgcgacccatacacccaaaggattgaggtcttggacaacagatggctaagcagcttggcgtaactagatct

SEQ ID NO: 82 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgcgctacgacccatacacccaaaggattaaaaaaaaagctgccatcagtcggcgtggactgtagaacactgccaatgccggtcccaagcccggataaaagtggagggtacagtccacgctttttttttgctgccacaatacctcggcccttctcagttcgctgcgacccatacacccaaaggattgaggtcttggacagcacgcgctaagcagcttggcgtaactagatct

SEQ ID NO: 83 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgcgttcgctacgacccatacacccaaaggattaaaaaaaaagctgccatcagtcggcgtggactgtagaacactgccaatgccggtcccaagcccggataaaagtggagggtacagtccacgcttttttttgccacaatacctcggcccttctcagttcgctgcgacccatacacccaaaggattgaggtcttggacaatcggggctaagcagcttggcgtaactagatct

SEQ ID NO: 84 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgctcagttcgctacgacccatacacccaaaggattaaaaaaaaagctgccatcagtcggcgtggactgtagaacactgccaatgccggtcccaagcccggataaaagtggagggtacagtccacgctttttttcacaatacctcggcccttctcagttcgctgcgacccatacacccaaaggattgaggtcttggacacgagttgctaagcagcttggcgtaactagatct

SEQ ID NO: 85 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgcccttctcagttcgctacgacccatacacccaaaggattaaaaaaaaagctgccatcagtcggcgtggactgtagaacactgccaatgccggtcccaagcccggataaaagtggagggtacagtccacgcttttttttacctcggcccttctcagttcgctgcgacccatacacccaaaggattgaggtcttggacagggggagctaagcagcttggcgtaactagatct

SEQ ID NO: 86 - DNA, 350 bp, unidentified
  source 1..350: mol_type = unassigned DNA
ggccaaagcatgcatgtgccacgtaatagaggggcgtttcagagctatgctggaaacagcatagcaagttgaaataaggctagtccgttatcaacttgaaaaagtggcaccgagtcggtgctcacggagttccagcccctctattacgtgaaaaaaaaagctgccatcagtcggcgtggactgtagaacactgccaatgccggtcccaagcccggataaaagtggagggtacagtccacgctttttttagaagacagccatccaaaattacactgtcatggagttccagcccctctattacgtggcagagagttttacgttctgctaagcagcttggcgtaactagatct

SEQ ID NO: 87 - DNA, 350 bp, unidentified
  source 1..350: mol_type = unassigned DNA
ggccaaagcatgcatgtgccacgtaatagaggggcgtttcagagctatgctggaaacagcatagcaagttgaaataaggctagtccgttatcaacttgaaaaagtggcaccgagtcggtgctacactgtcacggagttccagcccctctattacgtgaaaaaaaaagctgccatcagtcggcgtggactgtagaacactgccaatgccggtcccaagcccggataaaagtggagggtacagtccacgctttttttagccatccaaaattacactgtcatggagttccagcccctctattacgtggcagagagttttaaagttggctaagcagcttggcgtaactagatct

SEQ ID NO: 88 - DNA, 350 bp, unidentified
  source 1..350: mol_type = unassigned DNA
ggccaaagcatgcatgtgccacgtaatagaggggcgtttcagagctatgctggaaacagcatagcaagttgaaataaggctagtccgttatcaacttgaaaaagtggcaccgagtcggtgcaaaattacactgtcacggagttccagcccctctattacgtgaaaaaaaaagctgccatcagtcggcgtggactgtagaacactgccaatgccggtcccaagcccggataaaagtggagggtacagtccacgcttttttttccaaaattacactgtcatggagttccagcccctctattacgtggcagagagttttagattgtgctaagcagcttggcgtaactagatct

SEQ ID NO: 89 - DNA, 350 bp, unidentified
  source 1..350: mol_type = unassigned DNA
ggccaaagcatgcatgatccaaaattacactgtcagtttcagagctatgctggaaacagcatagcaagttgaaataaggctagtccgttatcaacttgaaaaagtggcaccgagtcggtgctccgtgacagtgtaattttgaaaaaaaaagctgccatcagtcggcgtggactgtagaacactgccaatgccggtcccaagcccggataaaagtggagggtacagtccacgctttttttttggcatcattaaaactctctgccacgtaatagaggggctggaactccatgacagtgtaattttggatggctgtcttcaacaccgctaagcagcttggcgtaactagatct

SEQ ID NO: 90 - DNA, 350 bp, unidentified
  source 1..350: mol_type = unassigned DNA
ggccaaagcatgcatgatccaaaattacactgtcagtttcagagctatgctggaaacagcatagcaagttgaaataaggctagtccgttatcaacttgaaaaagtggcaccgagtcggtgcgaactccgtgacagtgtaattttgaaaaaaaaagctgccatcagtcggcgtggactgtagaacactgccaatgccggtcccaagcccggataaaagtggagggtacagtccacgctttttttcatcattaaaactctctgccacgtaatagaggggctggaactccatgacagtgtaattttggatggctgtcttctataatgctaagcagcttggcgtaactagatct

SEQ ID NO: 91 - DNA, 350 bp, unidentified
  source 1..350: mol_type = unassigned DNA
ggccaaagcatgcatgatccaaaattacactgtcagtttcagagctatgctggaaacagcatagcaagttgaaataaggctagtccgttatcaacttgaaaaagtggcaccgagtcggtgcctggaactccgtgacagtgtaattttgaaaaaaaaagctgccatcagtcggcgtggactgtagaacactgccaatgccggtcccaagcccggataaaagtggagggtacagtccacgctttttttcattaaaactctctgccacgtaatagaggggctggaactccatgacagtgtaattttggatggctgtcttccagtcagctaagcagcttggcgtaactagatct

SEQ ID NO: 92 - DNA, 350 bp, unidentified
  source 1..350: mol_type = unassigned DNA
ggccaaagcatgcatgatccaaaattacactgtcagtttcagagctatgctggaaacagcatagcaagttgaaataaggctagtccgttatcaacttgaaaaagtggcaccgagtcggtgcaggggctggaactccgtgacagtgtaattttgaaaaaaaaagctgccatcagtcggcgtggactgtagaacactgccaatgccggtcccaagcccggataaaagtggagggtacagtccacgctttttttaaactctctgccacgtaatagaggggctggaactccatgacagtgtaattttggatggctgtcttccacatagctaagcagcttggcgtaactagatct

SEQ ID NO: 93 - DNA, 350 bp, unidentified
  source 1..350: mol_type = unassigned DNA
ggccaaagcatgcatggagaagggccgaggtattggtttcagagctatgctggaaacagcatagcaagttgaaataaggctagtccgttatcaacttgaaaaagtggcaccgagtcggtgcttgctgccacaatacctcggcccttaaaaaaaaagctgccatcagtcggcgtggactgtagaacactgccaatgccggtcccaagcccggataaaagtggagggtacagtccacgctttttttcttcactcaagcctgtggttttggtcttaggaactttgttgccacaatacctcggcccttctcagttcgctacagatcagctaagcagcttggcgtaactagatct

SEQ ID NO: 94 - DNA, 350 bp, unidentified
  source 1..350: mol_type = unassigned DNA
ggccaaagcatgcatggagaagggccgaggtattggtttcagagctatgctggaaacagcatagcaagttgaaataaggctagtccgttatcaacttgaaaaagtggcaccgagtcggtgcaactttgctgccacaatacctcggcccttaaaaaaaaagctgccatcagtcggcgtggactgtagaacactgccaatgccggtcccaagcccggataaaagtggagggtacagtccacgctttttttactcaagcctgtggttttggtcttaggaactttgttgccacaatacctcggcccttctcagttcgctaccgagatgctaagcagcttggcgtaactagatct

SEQ ID NO: 95 - DNA, 350 bp, unidentified
  source 1..350: mol_type = unassigned DNA
ggccaaagcatgcatggagaagggccgaggtattggtttcagagctatgctggaaacagcatagcaagttgaaataaggctagtccgttatcaacttgaaaaagtggcaccgagtcggtgcaggaactttgctgccacaatacctcggcccttaaaaaaaaagctgccatcagtcggcgtggactgtagaacactgccaatgccggtcccaagcccggataaaagtggagggtacagtccacgctttttttcaagcctgtggttttggtcttaggaactttgttgccacaatacctcggcccttctcagttcgctacggtcgtgctaagcagcttggcgtaactagatct

SEQ ID NO: 96 - DNA, 350 bp, unidentified
  source 1..350: mol_type = unassigned DNA
ggccaaagcatgcatgagcctgtggttttggtcttgtttcagagctatgctggaaacagcatagcaagttgaaataaggctagtccgttatcaacttgaaaaagtggcaccgagtcggtgctgtggcagcaaagttcctaagaccaaaaccacagaaaaaaaaagctgccatcagtcggcgtggactgtagaacactgccaatgccggtcccaagcccggataaaagtggagggtacagtccacgctttttttgagaagggccgaggtattgtggcaacaaagttcctaagaccaaaaccacaggcttgagtgaagggctggcgctaagcagcttggcgtaactagatct

SEQ ID NO: 97 - DNA, 350 bp, unidentified
  source 1..350: mol_type = unassigned DNA
ggccaaagcatgcatgagcctgtggttttggtcttgtttcagagctatgctggaaacagcatagcaagttgaaataaggctagtccgttatcaacttgaaaaagtggcaccgagtcggtgctattgtggcagcaaagttcctaagaccaaaaccacagaaaaaaaaagctgccatcagtcggcgtggactgtagaacactgccaatgccggtcccaagcccggataaaagtggagggtacagtccacgctttttttaagggccgaggtattgtggcaacaaagttcctaagaccaaaaccacaggcttgagtgaaggaggcttgctaagcagcttggcgtaactagatct

SEQ ID NO: 98 - DNA, 350 bp, unidentified
  source 1..350: mol_type = unassigned DNA
ggccaaagcatgcatgagcctgtggttttggtcttgtttcagagctatgctggaaacagcatagcaagttgaaataaggctagtccgttatcaacttgaaaaagtggcaccgagtcggtgccgaggtattgtggcagcaaagttcctaagaccaaaaccacagaaaaaaaaagctgccatcagtcggcgtggactgtagaacactgccaatgccggtcccaagcccggataaaagtggagggtacagtccacgctttttttccgaggtattgtggcaacaaagttcctaagaccaaaaccacaggcttgagtgaagggaattagctaagcagcttggcgtaactagatct

SEQ ID NO: 99 - DNA, 350 bp, unidentified
  source 1..350: mol_type = unassigned DNA
ggccaaagcatgcatgagaaggtctagattcagtggtttcagagctatgctggaaacagcatagcaagttgaaataaggctagtccgttatcaacttgaaaaagtggcaccgagtcggtgcacattgaatctagacctaaaaaaaaagctgccatcagtcggcgtggactgtagaacactgccaatgccggtcccaagcccggataaaagtggagggtacagtccacgctttttttctcctcaccctccccattctctcttctaggagaatgatgtaaacctgacccacactgaatctagaccttctcgtttaaagacaaggagctaagcagcttggcgtaactagatct

SEQ ID NO: 100 - DNA, 350 bp, unidentified
  source 1..350: mol_type = unassigned DNA
ggccaaagcatgcatgagaaggtctagattcagtggtttcagagctatgctggaaacagcatagcaagttgaaataaggctagtccgttatcaacttgaaaaagtggcaccgagtcggtgcacccacattgaatctagacctaaaaaaaaagctgccatcagtcggcgtggactgtagaacactgccaatgccggtcccaagcccggataaaagtggagggtacagtccacgcttttttttcaccctccccattctctcttctaggagaatgatgtaaacctgacccacactgaatctagaccttctcgtttaaagactgctagctaagcagcttggcgtaactagatct

SEQ ID NO: 101 - DNA, 350 bp, unidentified
  source 1..350: mol_type = unassigned DNA
ggccaaagcatgcatgagaaggtctagattcagtggtttcagagctatgctggaaacagcatagcaagttgaaataaggctagtccgttatcaacttgaaaaagtggcaccgagtcggtgcctgacccacattgaatctagacctaaaaaaaaagctgccatcagtcggcgtggactgtagaacactgccaatgccggtcccaagcccggataaaagtggagggtacagtccacgctttttttccctccccattctctcttctaggagaatgatgtaaacctgacccacactgaatctagaccttctcgtttaaagagctcacgctaagcagcttggcgtaactagatct

SEQ ID NO: 102 - DNA, 350 bp, unidentified
  source 1..350: mol_type = unassigned DNA
ggccaaagcatgcatgagaaggtctagattcagtggtttcagagctatgctggaaacagcatagcaagttgaaataaggctagtccgttatcaacttgaaaaagtggcaccgagtcggtgctaaacctgacccacattgaatctagacctaaaaaaaaagctgccatcagtcggcgtggactgtagaacactgccaatgccggtcccaagcccggataaaagtggagggtacagtccacgctttttttcccattctctcttctaggagaatgatgtaaacctgacccacactgaatctagaccttctcgtttaaagagaaactgctaagcagcttggcgtaactagatct

SEQ ID NO: 103 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgctccgagtcttccactgcacacagtacatcaaaaaaaaagctgccatcagtcggcgtggactgtagaacactgccaatgccggtcccaagcccggataaaagtggagggtacagtccacgctttttttctcgggatttcttgggtggcctggccttccaagtcttccactgcacacagtacatcagacatggatccaaagtgagctaagcagcttggcgtaactagatct

SEQ ID NO: 104 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgcgccttccgagtcttccactgcacacagtacatcaaaaaaaaagctgccatcagtcggcgtggactgtagaacactgccaatgccggtcccaagcccggataaaagtggagggtacagtccacgctttttttggatttcttgggtggcctggccttccaagtcttccactgcacacagtacatcagacatggatccacttctcgctaagcagcttggcgtaactagatct

SEQ ID NO: 105 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgcctggccttccgagtcttccactgcacacagtacatcaaaaaaaaagctgccatcagtcggcgtggactgtagaacactgccaatgccggtcccaagcccggataaaagtggagggtacagtccacgcttttttttttcttgggtggcctggccttccaagtcttccactgcacacagtacatcagacatggatccaaccggtgctaagcagcttggcgtaactagatct

SEQ ID NO: 106 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgcgtggcctggccttccgagtcttccactgcacacagtacatcaaaaaaaaagctgccatcagtcggcgtggactgtagaacactgccaatgccggtcccaagcccggataaaagtggagggtacagtccacgcttttttttgggtggcctggccttccaagtcttccactgcacacagtacatcagacatggatccatgatcggctaagcagcttggcgtaactagatct

SEQ ID NO: 107 - DNA, 350 bp, unidentified
  source 1..350: mol_type = unassigned DNA
ggccaaagcatgcatgctgtgtgcagtggaagactgtttcagagctatgctggaaacagcatagcaagttgaaataaggctagtccgttatcaacttgaaaaagtggcaccgagtcggtgctccgagtcttccactgcacaaaaaaaaaagctgccatcagtcggcgtggactgtagaacactgccaatgccggtcccaagcccggataaaagtggagggtacagtccacgcttttttttggctggcctgctttcctctcgggatttcttgggtggcctggccttccaagtcttccactgcacacagtacatcagacggtcgggctaagcagcttggcgtaactagatct

SEQ ID NO: 108 - DNA, 350 bp, unidentified
  source 1..350: mol_type = unassigned DNA
ggccaaagcatgcatgctgtgtgcagtggaagactgtttcagagctatgctggaaacagcatagcaagttgaaataaggctagtccgttatcaacttgaaaaagtggcaccgagtcggtgcgccttccgagtcttccactgcacaaaaaaaaaagctgccatcagtcggcgtggactgtagaacactgccaatgccggtcccaagcccggataaaagtggagggtacagtccacgcttttttttggcctgctttcctctcgggatttcttgggtggcctggccttccaagtcttccactgcacacagtacatcagactggaccgctaagcagcttggcgtaactagatct

SEQ ID NO: 109 - DNA, 350 bp, unidentified
  source 1..350: mol_type = unassigned DNA
ggccaaagcatgcatgctgtgtgcagtggaagactgtttcagagctatgctggaaacagcatagcaagttgaaataaggctagtccgttatcaacttgaaaaagtggcaccgagtcggtgcctggccttccgagtcttccactgcacaaaaaaaaaagctgccatcagtcggcgtggactgtagaacactgccaatgccggtcccaagcccggataaaagtggagggtacagtccacgctttttttcctgctttcctctcgggatttcttgggtggcctggccttccaagtcttccactgcacacagtacatcagactagctagctaagcagcttggcgtaactagatct

SEQ ID NO: 110 - DNA, 350 bp, unidentified
  source 1..350: mol_type = unassigned DNA
ggccaaagcatgcatgctgtgtgcagtggaagactgtttcagagctatgctggaaacagcatagcaagttgaaataaggctagtccgttatcaacttgaaaaagtggcaccgagtcggtgcgtggcctggccttccgagtcttccactgcacaaaaaaaaaagctgccatcagtcggcgtggactgtagaacactgccaatgccggtcccaagcccggataaaagtggagggtacagtccacgcttttttttttcctctcgggatttcttgggtggcctggccttccaagtcttccactgcacacagtacatcagaccataatgctaagcagcttggcgtaactagatct

SEQ ID NO: 111 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgcactcggaaggccaggccacccaagaaatcaaaaaaaaagctgccatcagtcggcgtggactgtagaacactgccaatgccggtcccaagcccggataaaagtggagggtacagtccacgcttttttttgtctgatgtactgtgtgcagtggaagacttggaaggccaggccacccaagaaatcccgagaggaaagcagaatagctaagcagcttggcgtaactagatct

SEQ ID NO: 112 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgcgaagactcggaaggccaggccacccaagaaatcaaaaaaaaagctgccatcagtcggcgtggactgtagaacactgccaatgccggtcccaagcccggataaaagtggagggtacagtccacgcttttttttgatgtactgtgtgcagtggaagacttggaaggccaggccacccaagaaatcccgagaggaaagcgaaccggctaagcagcttggcgtaactagatct

SEQ ID NO: 113 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgcgtggaagactcggaaggccaggccacccaagaaatcaaaaaaaaagctgccatcagtcggcgtggactgtagaacactgccaatgccggtcccaagcccggataaaagtggagggtacagtccacgcttttttttgtactgtgtgcagtggaagacttggaaggccaggccacccaagaaatcccgagaggaaagcgcgaacgctaagcagcttggcgtaactagatct

SEQ ID NO: 114 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgcgtgcagtggaagactcggaaggccaggccacccaagaaatcaaaaaaaaagctgccatcagtcggcgtggactgtagaacactgccaatgccggtcccaagcccggataaaagtggagggtacagtccacgcttttttttgtgtgcagtggaagacttggaaggccaggccacccaagaaatcccgagaggaaagcgataccgctaagcagcttggcgtaactagatct

SEQ ID NO: 115 - DNA, 350 bp, unidentified
  source 1..350: mol_type = unassigned DNA
ggccaaagcatgcatgattccttacctgggaaaacgtttcagagctatgctggaaacagcatagcaagttgaaataaggctagtccgttatcaacttgaaaaagtggcaccgagtcggtgctttgcccagttttcccaggtaaggaaaaaaaaagctgccatcagtcggcgtggactgtagaacactgccaatgccggtcccaagcccggataaaagtggagggtacagtccacgctttttttgctgttgggacatgtgcccttgttttcagatcgcagcttttcccagttttcccaggtaaggaatggattttttatcaagcgctaagcagcttggcgtaactagatct

SEQ ID NO: 116 - DNA, 350 bp, unidentified
  source 1..350: mol_type = unassigned DNA
ggccaaagcatgcatgattccttacctgggaaaacgtttcagagctatgctggaaacagcatagcaagttgaaataaggctagtccgttatcaacttgaaaaagtggcaccgagtcggtgccagctttgcccagttttcccaggtaaggaaaaaaaaagctgccatcagtcggcgtggactgtagaacactgccaatgccggtcccaagcccggataaaagtggagggtacagtccacgctttttttttgggacatgtgcccttgttttcagatcgcagcttttcccagttttcccaggtaaggaatggattttttagactacgctaagcagcttggcgtaactagatct

SEQ ID NO: 117 - DNA, 350 bp, unidentified
  source 1..350: mol_type = unassigned DNA
ggccaaagcatgcatgattccttacctgggaaaacgtttcagagctatgctggaaacagcatagcaagttgaaataaggctagtccgttatcaacttgaaaaagtggcaccgagtcggtgctcgcagctttgcccagttttcccaggtaaggaaaaaaaaagctgccatcagtcggcgtggactgtagaacactgccaatgccggtcccaagcccggataaaagtggagggtacagtccacgctttttttggacatgtgcccttgttttcagatcgcagcttttcccagttttcccaggtaaggaatggattttttacatagagctaagcagcttggcgtaactagatct

SEQ ID NO: 118 - DNA, 350 bp, unidentified
  source 1..350: mol_type = unassigned DNA
ggccaaagcatgcatgattccttacctgggaaaacgtttcagagctatgctggaaacagcatagcaagttgaaataaggctagtccgttatcaacttgaaaaagtggcaccgagtcggtgctcagatcgcagctttgcccagttttcccaggtaaggaaaaaaaaagctgccatcagtcggcgtggactgtagaacactgccaatgccggtcccaagcccggataaaagtggagggtacagtccacgcttttttttgtgcccttgttttcagatcgcagcttttcccagttttcccaggtaaggaatggattttttagattccgctaagcagcttggcgtaactagatct

SEQ ID NO: 119 - DNA, 350 bp, unidentified
  source 1..350: mol_type = unassigned DNA
ggccaaagcatgcatgttccttacctgggaaaactgtttcagagctatgctggaaacagcatagcaagttgaaataaggctagtccgttatcaacttgaaaaagtggcaccgagtcggtgctttgcccagttttcccaggtaagaaaaaaaaagctgccatcagtcggcgtggactgtagaacactgccaatgccggtcccaagcccggataaaagtggagggtacagtccacgctttttttgagctgttgggacatgtgcccttgttttcagatcgcagcttttcccagttttcccaggtaaggaatggatttttttgccttgctaagcagcttggcgtaactagatct

SEQ ID NO: 120 - DNA, 350 bp, unidentified
  source 1..350: mol_type = unassigned DNA
ggccaaagcatgcatgttccttacctgggaaaactgtttcagagctatgctggaaacagcatagcaagttgaaataaggctagtccgttatcaacttgaaaaagtggcaccgagtcggtgccagctttgcccagttttcccaggtaagaaaaaaaaagctgccatcagtcggcgtggactgtagaacactgccaatgccggtcccaagcccggataaaagtggagggtacagtccacgcttttttttgttgggacatgtgcccttgttttcagatcgcagcttttcccagttttcccaggtaaggaatggatttttttgtctcgctaagcagcttggcgtaactagatct

SEQ ID NO: 121 - DNA, 350 bp, unidentified
  source 1..350: mol_type = unassigned DNA
ggccaaagcatgcatgttccttacctgggaaaactgtttcagagctatgctggaaacagcatagcaagttgaaataaggctagtccgttatcaacttgaaaaagtggcaccgagtcggtgctcgcagctttgcccagttttcccaggtaagaaaaaaaaagctgccatcagtcggcgtggactgtagaacactgccaatgccggtcccaagcccggataaaagtggagggtacagtccacgcttttttttgggacatgtgcccttgttttcagatcgcagcttttcccagttttcccaggtaaggaatggattttttggaggtgctaagcagcttggcgtaactagatct

SEQ ID NO: 122 - DNA, 350 bp, unidentified
  source 1..350: mol_type = unassigned DNA
ggccaaagcatgcatgcctcaatcctttgggtgtagtttcagagctatgctggaaacagcatagcaagttgaaataaggctagtccgttatcaacttgaaaaagtggcaccgagtcggtgctacgacccatacacccaaaggattgaaaaaaaaagctgccatcagtcggcgtggactgtagaacactgccaatgccggtcccaagcccggataaaagtggagggtacagtccacgctttttttttgctgccacaatacctcggcccttctcagttcgctacaacccatacacccaaaggattgaggtcttggacaacctgcagctaagcagcttggcgtaactagatct

SEQ ID NO: 123 - DNA, 350 bp, unidentified
  source 1..350: mol_type = unassigned DNA
ggccaaagcatgcatgcctcaatcctttgggtgtagtttcagagctatgctggaaacagcatagcaagttgaaataaggctagtccgttatcaacttgaaaaagtggcaccgagtcggtgctcgctacgacccatacacccaaaggattgaaaaaaaaagctgccatcagtcggcgtggactgtagaacactgccaatgccggtcccaagcccggataaaagtggagggtacagtccacgcttttttttgccacaatacctcggcccttctcagttcgctacaacccatacacccaaaggattgaggtcttggacaaaacgccgctaagcagcttggcgtaactagatct

SEQ ID NO: 124 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgctacgacccatacacccaaaggattaaaaaaaaagctgccatcagtcggcgtggactgtagaacactgccaatgccggtcccaagcccggataaaagtggagggtacagtccacgctttttttctttgctgccacaatacctcggcccttctcagttcgctacaacccatacacccaaaggattgaggtcttggacacgtttcgctaagcagcttggcgtaactagatct

SEQ ID NO: 125 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgctcgctacgacccatacacccaaaggattaaaaaaaaagctgccatcagtcggcgtggactgtagaacactgccaatgccggtcccaagcccggataaaagtggagggtacagtccacgctttttttgctgccacaatacctcggcccttctcagttcgctacaacccatacacccaaaggattgaggtcttggacagtggcagctaagcagcttggcgtaactagatct

SEQ ID NO: 126 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgcagttcgctacgacccatacacccaaaggattaaaaaaaaagctgccatcagtcggcgtggactgtagaacactgccaatgccggtcccaagcccggataaaagtggagggtacagtccacgctttttttgccacaatacctcggcccttctcagttcgctacaacccatacacccaaaggattgaggtcttggacacacactgctaagcagcttggcgtaactagatct

SEQ ID NO: 127 - DNA, 350 bp, unidentified
  source 1..350: mol_type = unassigned DNA
ggccaaagcatgcatgctcaatcctttgggtgtatgtttcagagctatgctggaaacagcatagcaagttgaaataaggctagtccgttatcaacttgaaaaagtggcaccgagtcggtgcttctcagttcgctacgacccatacacccaaaggattaaaaaaaaagctgccatcagtcggcgtggactgtagaacactgccaatgccggtcccaagcccggataaaagtggagggtacagtccacgctttttttaatacctcggcccttctcagttcgctacaacccatacacccaaaggattgaggtcttggacaaatgcagctaagcagcttggcgtaactagatct

SEQ ID NO: 128 - DNA, 350 bp, unidentified
  source 1..350: mol_type = unassigned DNA
ggccaaagcatgcatgagctggaggacagtactcagtttcagagctatgctggaaacagcatagcaagttgaaataaggctagtccgttatcaacttgaaaaagtggcaccgagtcggtgccccgaaccgtgagtactgtcctccaaaaaaaaaagctgccatcagtcggcgtggactgtagaacactgccaatgccggtcccaagcccggataaaagtggagggtacagtccacgctttttttagtacatcagacatggatccaagcccatgtatacccccaaaccgtgagtactgtcctccagctaccagttgcccactcagctaagcagcttggcgtaactagatct

SEQ ID NO: 129 - DNA, 350 bp, unidentified
  source 1..350: mol_type = unassigned DNA
ggccaaagcatgcatgagctggaggacagtactcagtttcagagctatgctggaaacagcatagcaagttgaaataaggctagtccgttatcaacttgaaaaagtggcaccgagtcggtgctacccccgaaccgtgagtactgtcctccaaaaaaaaaagctgccatcagtcggcgtggactgtagaacactgccaatgccggtcccaagcccggataaaagtggagggtacagtccacgctttttttcatcagacatggatccaagcccatgtatacccccaaaccgtgagtactgtcctccagctaccagttgccggtggggctaagcagcttggcgtaactagatct

SEQ ID NO: 130 - DNA, 350 bp, unidentified
  source 1..350: mol_type = unassigned DNA
ggccaaagcatgcatgaggacagtactcacggtttgtttcagagctatgctggaaacagcatagcaagttgaaataaggctagtccgttatcaacttgaaaaagtggcaccgagtcggtgccccgaaccgtgagtactgtaaaaaaaaagctgccatcagtcggcgtggactgtagaacactgccaatgccggtcccaagcccggataaaagtggagggtacagtccacgcttttttttccactgcacacagtacatcagacatggatccaagcccatgtatacccccaaaccgtgagtactgtcctccagctaccactcaaagctaagcagcttggcgtaactagatct

SEQ ID NO: 131 - DNA, 350 bp, unidentified
  source 1..350: mol_type = unassigned DNA
ggccaaagcatgcatgaggacagtactcacggtttgtttcagagctatgctggaaacagcatagcaagttgaaataaggctagtccgttatcaacttgaaaaagtggcaccgagtcggtgctacccccgaaccgtgagtactgtaaaaaaaaagctgccatcagtcggcgtggactgtagaacactgccaatgccggtcccaagcccggataaaagtggagggtacagtccacgctttttttctgcacacagtacatcagacatggatccaagcccatgtatacccccaaaccgtgagtactgtcctccagctaccatgaagcgctaagcagcttggcgtaactagatct

SEQ ID NO: 132 - DNA, 350 bp, unidentified
  source 1..350: mol_type = unassigned DNA
ggccaaagcatgcatgaggacagtactcacggtttgtttcagagctatgctggaaacagcatagcaagttgaaataaggctagtccgttatcaacttgaaaaagtggcaccgagtcggtgcgtatacccccgaaccgtgagtactgtaaaaaaaaagctgccatcagtcggcgtggactgtagaacactgccaatgccggtcccaagcccggataaaagtggagggtacagtccacgctttttttcacacagtacatcagacatggatccaagcccatgtatacccccaaaccgtgagtactgtcctccagctaccagatgtggctaagcagcttggcgtaactagatct

SEQ ID NO: 133 - DNA, 350 bp, unidentified
  source 1..350: mol_type = unassigned DNA
ggccaaagcatgcatgaggacagtactcacggtttgtttcagagctatgctggaaacagcatagcaagttgaaataaggctagtccgttatcaacttgaaaaagtggcaccgagtcggtgccccatgtatacccccgaaccgtgagtactgtaaaaaaaaagctgccatcagtcggcgtggactgtagaacactgccaatgccggtcccaagcccggataaaagtggagggtacagtccacgctttttttagtacatcagacatggatccaagcccatgtatacccccaaaccgtgagtactgtcctccagctaccaagccatgctaagcagcttggcgtaactagatct

SEQ ID NO: 134 - DNA, 350 bp, unidentified
  source 1..350: mol_type = unassigned DNA
ggccaaagcatgcatgggacagtactcacggtttggtttcagagctatgctggaaacagcatagcaagttgaaataaggctagtccgttatcaacttgaaaaagtggcaccgagtcggtgccccgaaccgtgagtactgaaaaaaaaagctgccatcagtcggcgtggactgtagaacactgccaatgccggtcccaagcccggataaaagtggagggtacagtccacgctttttttcttccactgcacacagtacatcagacatggatccaagcccatgtatacccccaaaccgtgagtactgtcctccagctaccctctatgctaagcagcttggcgtaactagatct

SEQ ID NO: 135 - DNA, 350 bp, unidentified
  source 1..350: mol_type = unassigned DNA
ggccaaagcatgcatgggacagtactcacggtttggtttcagagctatgctggaaacagcatagcaagttgaaataaggctagtccgttatcaacttgaaaaagtggcaccgagtcggtgctacccccgaaccgtgagtactgaaaaaaaaagctgccatcagtcggcgtggactgtagaacactgccaatgccggtcccaagcccggataaaagtggagggtacagtccacgctttttttcactgcacacagtacatcagacatggatccaagcccatgtatacccccaaaccgtgagtactgtcctccagctaccgtatgggctaagcagcttggcgtaactagatct

SEQ ID NO: 136 - DNA, 350 bp, unidentified
  source 1..350: mol_type = unassigned DNA
ggccaaagcatgcatgggacagtactcacggtttggtttcagagctatgctggaaacagcatagcaagttgaaataaggctagtccgttatcaacttgaaaaagtggcaccgagtcggtgcgtatacccccgaaccgtgagtactgaaaaaaaaagctgccatcagtcggcgtggactgtagaacactgccaatgccggtcccaagcccggataaaagtggagggtacagtccacgcttttttttgcacacagtacatcagacatggatccaagcccatgtatacccccaaaccgtgagtactgtcctccagctacccctgttgctaagcagcttggcgtaactagatct

SEQ ID NO: 137 - DNA, 350 bp, unidentified
  source 1..350: mol_type = unassigned DNA
ggccaaagcatgcatgggacagtactcacggtttggtttcagagctatgctggaaacagcatagcaagttgaaataaggctagtccgttatcaacttgaaaaagtggcaccgagtcggtgccccatgtatacccccgaaccgtgagtactgaaaaaaaaagctgccatcagtcggcgtggactgtagaacactgccaatgccggtcccaagcccggataaaagtggagggtacagtccacgctttttttacagtacatcagacatggatccaagcccatgtatacccccaaaccgtgagtactgtcctccagctaccatttaggctaagcagcttggcgtaactagatct

SEQ ID NO: 138 - DNA, 350 bp, unidentified
  source 1..350: mol_type = unassigned DNA
ggccaaagcatgcatgctctgataagcagtactgtgtttcagagctatgctggaaacagcatagcaagttgaaataaggctagtccgttatcaacttgaaaaagtggcaccgagtcggtgccttggggcctacagtactgcttatcaaaaaaaaaagctgccatcagtcggcgtggactgtagaacactgccaatgccggtcccaagcccggataaaagtggagggtacagtccacgcttttttttgatcctgatttaacagtgataataacttttcacttagggcctacagtactgcttatcagagaagccaaagccaccaggctaagcagcttggcgtaactagatct

SEQ ID NO: 139 - DNA, 350 bp, unidentified
  source 1..350: mol_type = unassigned DNA
ggccaaagcatgcatgctctgataagcagtactgtgtttcagagctatgctggaaacagcatagcaagttgaaataaggctagtccgttatcaacttgaaaaagtggcaccgagtcggtgcttcacttggggcctacagtactgcttatcaaaaaaaaaagctgccatcagtcggcgtggactgtagaacactgccaatgccggtcccaagcccggataaaagtggagggtacagtccacgctttttttcctgatttaacagtgataataacttttcacttagggcctacagtactgcttatcagagaagccaaagctaagcagctaagcagcttggcgtaactagatct

SEQ ID NO: 140 - DNA, 350 bp, unidentified
  source 1..350: mol_type = unassigned DNA
ggccaaagcatgcatgctctgataagcagtactgtgtttcagagctatgctggaaacagcatagcaagttgaaataaggctagtccgttatcaacttgaaaaagtggcaccgagtcggtgccttttcacttggggcctacagtactgcttatcaaaaaaaaaagctgccatcagtcggcgtggactgtagaacactgccaatgccggtcccaagcccggataaaagtggagggtacagtccacgctttttttgatttaacagtgataataacttttcacttagggcctacagtactgcttatcagagaagccaaagccaagtggctaagcagcttggcgtaactagatct

SEQ ID NO: 141 - DNA, 350 bp, unidentified
  source 1..350: mol_type = unassigned DNA
ggccaaagcatgcatgtgataataacttttcacttgtttcagagctatgctggaaacagcatagcaagttgaaataaggctagtccgttatcaacttgaaaaagtggcaccgagtcggtgcccccaagtgaaaagttattaaaaaaaaaagctgccatcagtcggcgtggactgtagaacactgccaatgccggtcccaagcccggataaaagtggagggtacagtccacgcttttttttccagggggagaagctttggcttctctgataagcagtactgtaggccctaagtgaaaagttattatcactgttaaatcatcgacgctaagcagcttggcgtaactagatct

SEQ ID NO: 142 - DNA, 350 bp, unidentified
  source 1..350: mol_type = unassigned DNA
ggccaaagcatgcatgtgataataacttttcacttgtttcagagctatgctggaaacagcatagcaagttgaaataaggctagtccgttatcaacttgaaaaagtggcaccgagtcggtgctaggccccaagtgaaaagttattaaaaaaaaaagctgccatcagtcggcgtggactgtagaacactgccaatgccggtcccaagcccggataaaagtggagggtacagtccacgctttttttgggggagaagctttggcttctctgataagcagtactgtaggccctaagtgaaaagttattatcactgttaaatcatcatggctaagcagcttggcgtaactagatct

SEQ ID NO: 143 - DNA, 350 bp, unidentified
  source 1..350: mol_type = unassigned DNA
ggccaaagcatgcatgtgataataacttttcacttgtttcagagctatgctggaaacagcatagcaagttgaaataaggctagtccgttatcaacttgaaaaagtggcaccgagtcggtgcctgtaggccccaagtgaaaagttattaaaaaaaaaagctgccatcagtcggcgtggactgtagaacactgccaatgccggtcccaagcccggataaaagtggagggtacagtccacgctttttttggagaagctttggcttctctgataagcagtactgtaggccctaagtgaaaagttattatcactgttaaatcccggtggctaagcagcttggcgtaactagatct

SEQ ID NO: 144 - DNA, 350 bp, unidentified
  source 1..350: mol_type = unassigned DNA
ggccaaagcatgcatgtgataataacttttcacttgtttcagagctatgctggaaacagcatagcaagttgaaataaggctagtccgttatcaacttgaaaaagtggcaccgagtcggtgccagtactgtaggccccaagtgaaaagttattaaaaaaaaaagctgccatcagtcggcgtggactgtagaacactgccaatgccggtcccaagcccggataaaagtggagggtacagtccacgctttttttagctttggcttctctgataagcagtactgtaggccctaagtgaaaagttattatcactgttaaatcccttcagctaagcagcttggcgtaactagatct

SEQ ID NO: 145 - DNA, 350 bp, unidentified
  source 1..350: mol_type = unassigned DNA
ggccaaagcatgcatggataataacttttcacttagtttcagagctatgctggaaacagcatagcaagttgaaataaggctagtccgttatcaacttgaaaaagtggcaccgagtcggtgcccccaagtgaaaagttattaaaaaaaaagctgccatcagtcggcgtggactgtagaacactgccaatgccggtcccaagcccggataaaagtggagggtacagtccacgctttttttgctccagggggagaagctttggcttctctgataagcagtactgtaggccctaagtgaaaagttattatcactgttaaatgggacagctaagcagcttggcgtaactagatct

SEQ ID NO: 146 - DNA, 350 bp, unidentified
  source 1..350: mol_type = unassigned DNA
ggccaaagcatgcatggataataacttttcacttagtttcagagctatgctggaaacagcatagcaagttgaaataaggctagtccgttatcaacttgaaaaagtggcaccgagtcggtgctaggccccaagtgaaaagttattaaaaaaaaagctgccatcagtcggcgtggactgtagaacactgccaatgccggtcccaagcccggataaaagtggagggtacagtccacgctttttttcagggggagaagctttggcttctctgataagcagtactgtaggccctaagtgaaaagttattatcactgttaaatccatgagctaagcagcttggcgtaactagatct

SEQ ID NO: 147 - DNA, 350 bp, unidentified
  source 1..350: mol_type = unassigned DNA
ggccaaagcatgcatggataataacttttcacttagtttcagagctatgctggaaacagcatagcaagttgaaataaggctagtccgttatcaacttgaaaaagtggcaccgagtcggtgcctgtaggccccaagtgaaaagttattaaaaaaaaagctgccatcagtcggcgtggactgtagaacactgccaatgccggtcccaagcccggataaaagtggagggtacagtccacgctttttttggggagaagctttggcttctctgataagcagtactgtaggccctaagtgaaaagttattatcactgttaaattcttatgctaagcagcttggcgtaactagatct

SEQ ID NO: 148 - DNA, 350 bp, unidentified
  source 1..350: mol_type = unassigned DNA
ggccaaagcatgcatggataataacttttcacttagtttcagagctatgctggaaacagcatagcaagttgaaataaggctagtccgttatcaacttgaaaaagtggcaccgagtcggtgccagtactgtaggccccaagtgaaaagttattaaaaaaaaagctgccatcagtcggcgtggactgtagaacactgccaatgccggtcccaagcccggataaaagtggagggtacagtccacgctttttttgaagctttggcttctctgataagcagtactgtaggccctaagtgaaaagttattatcactgttaaattgtcctgctaagcagcttggcgtaactagatct

SEQ ID NO: 149 - DNA, 350 bp, unidentified
  source 1..350: mol_type = unassigned DNA
ggccaaagcatgcatgttccagcccctctattacggtttcagagctatgctggaaacagcatagcaagttgaaataaggctagtccgttatcaacttgaaaaagtggcaccgagtcggtgcctctctgccacgtaatagaggggctgaaaaaaaaagctgccatcagtcggcgtggactgtagaacactgccaatgccggtcccaagcccggataaaagtggagggtacagtccacgctttttttcctcaccttactttctccttggcatcattaaaactccctgccacgtaatagaggggctggaactccgtgacaattgacgctaagcagcttggcgtaactagatct

SEQ ID NO: 150 - DNA, 350 bp, unidentified
  source 1..350: mol_type = unassigned DNA
ggccaaagcatgcatgttccagcccctctattacggtttcagagctatgctggaaacagcatagcaagttgaaataaggctagtccgttatcaacttgaaaaagtggcaccgagtcggtgcaaaactctctgccacgtaatagaggggctgaaaaaaaaagctgccatcagtcggcgtggactgtagaacactgccaatgccggtcccaagcccggataaaagtggagggtacagtccacgctttttttaccttactttctccttggcatcattaaaactccctgccacgtaatagaggggctggaactccgtgacaggccacgctaagcagcttggcgtaactagatct

SEQ ID NO: 151 - DNA, 350 bp, unidentified
  source 1..350: mol_type = unassigned DNA
ggccaaagcatgcatgttccagcccctctattacggtttcagagctatgctggaaacagcatagcaagttgaaataaggctagtccgttatcaacttgaaaaagtggcaccgagtcggtgcattaaaactctctgccacgtaatagaggggctgaaaaaaaaagctgccatcagtcggcgtggactgtagaacactgccaatgccggtcccaagcccggataaaagtggagggtacagtccacgctttttttttactttctccttggcatcattaaaactccctgccacgtaatagaggggctggaactccgtgacacctgtagctaagcagcttggcgtaactagatct

SEQ ID NO: 152 - DNA, 350 bp, unidentified
  source 1..350: mol_type = unassigned DNA
ggccaaagcatgcatgttccagcccctctattacggtttcagagctatgctggaaacagcatagcaagttgaaataaggctagtccgttatcaacttgaaaaagtggcaccgagtcggtgcgcatcattaaaactctctgccacgtaatagaggggctgaaaaaaaaagctgccatcagtcggcgtggactgtagaacactgccaatgccggtcccaagcccggataaaagtggagggtacagtccacgctttttttttctccttggcatcattaaaactccctgccacgtaatagaggggctggaactccgtgacagaacatgctaagcagcttggcgtaactagatct

SEQ ID NO: 153 - DNA, 350 bp, unidentified
  source 1..350: mol_type = unassigned DNA
ggccaaagcatgcatgagcccctctattacgtggcgtttcagagctatgctggaaacagcatagcaagttgaaataaggctagtccgttatcaacttgaaaaagtggcaccgagtcggtgcctctctgccacgtaatagagggaaaaaaaaagctgccatcagtcggcgtggactgtagaacactgccaatgccggtcccaagcccggataaaagtggagggtacagtccacgcttttttttgtcaccacctcaccttactttctccttggcatcattaaaactccctgccacgtaatagaggggctggaactccgtgtaaccgctaagcagcttggcgtaactagatct

SEQ ID NO: 154 - DNA, 350 bp, unidentified
  source 1..350: mol_type = unassigned DNA
ggccaaagcatgcatgagcccctctattacgtggcgtttcagagctatgctggaaacagcatagcaagttgaaataaggctagtccgttatcaacttgaaaaagtggcaccgagtcggtgcaaaactctctgccacgtaatagagggaaaaaaaaagctgccatcagtcggcgtggactgtagaacactgccaatgccggtcccaagcccggataaaagtggagggtacagtccacgctttttttaccacctcaccttactttctccttggcatcattaaaactccctgccacgtaatagaggggctggaactccgtggacgtgctaagcagcttggcgtaactagatct

SEQ ID NO: 155 - DNA, 350 bp, unidentified
  source 1..350: mol_type = unassigned DNA
ggccaaagcatgcatgagcccctctattacgtggcgtttcagagctatgctggaaacagcatagcaagttgaaataaggctagtccgttatcaacttgaaaaagtggcaccgagtcggtgcattaaaactctctgccacgtaatagagggaaaaaaaaagctgccatcagtcggcgtggactgtagaacactgccaatgccggtcccaagcccggataaaagtggagggtacagtccacgctttttttacctcaccttactttctccttggcatcattaaaactccctgccacgtaatagaggggctggaactccgtaaataggctaagcagcttggcgtaactagatct

SEQ ID NO: 156 - DNA, 350 bp, unidentified
  source 1..350: mol_type = unassigned DNA
ggccaaagcatgcatgagcccctctattacgtggcgtttcagagctatgctggaaacagcatagcaagttgaaataaggctagtccgttatcaacttgaaaaagtggcaccgagtcggtgcgcatcattaaaactctctgccacgtaatagagggaaaaaaaaagctgccatcagtcggcgtggactgtagaacactgccaatgccggtcccaagcccggataaaagtggagggtacagtccacgctttttttaccttactttctccttggcatcattaaaactccctgccacgtaatagaggggctggaactccgttgcgatgctaagcagcttggcgtaactagatct

SEQ ID NO: 157 - DNA, 350 bp, unidentified
  source 1..350: mol_type = unassigned DNA
ggccaaagcatgcatggcccctctattacgtggcagtttcagagctatgctggaaacagcatagcaagttgaaataaggctagtccgttatcaacttgaaaaagtggcaccgagtcggtgcctctctgccacgtaatagaggaaaaaaaaagctgccatcagtcggcgtggactgtagaacactgccaatgccggtcccaagcccggataaaagtggagggtacagtccacgcttttttttttgtcaccacctcaccttactttctccttggcatcattaaaactccctgccacgtaatagaggggctggaactccgtacattgctaagcagcttggcgtaactagatct

SEQ ID NO: 158 - DNA, 350 bp, unidentified
  source 1..350: mol_type = unassigned DNA
ggccaaagcatgcatggcccctctattacgtggcagtttcagagctatgctggaaacagcatagcaagttgaaataaggctagtccgttatcaacttgaaaaagtggcaccgagtcggtgcaaaactctctgccacgtaatagaggaaaaaaaaagctgccatcagtcggcgtggactgtagaacactgccaatgccggtcccaagcccggataaaagtggagggtacagtccacgcttttttttcaccacctcaccttactttctccttggcatcattaaaactccctgccacgtaatagaggggctggaactccgactaccgctaagcagcttggcgtaactagatct

SEQ ID NO: 159 - DNA, 350 bp, unidentified
  source 1..350: mol_type = unassigned DNA
ggccaaagcatgcatggcccctctattacgtggcagtttcagagctatgctggaaacagcatagcaagttgaaataaggctagtccgttatcaacttgaaaaagtggcaccgagtcggtgcattaaaactctctgccacgtaatagaggaaaaaaaaagctgccatcagtcggcgtggactgtagaacactgccaatgccggtcccaagcccggataaaagtggagggtacagtccacgctttttttccacctcaccttactttctccttggcatcattaaaactccctgccacgtaatagaggggctggaactccgggcgacgctaagcagcttggcgtaactagatct

SEQ ID NO: 160 - DNA, 350 bp, unidentified
  source 1..350: mol_type = unassigned DNA
ggccaaagcatgcatggcccctctattacgtggcagtttcagagctatgctggaaacagcatagcaagttgaaataaggctagtccgttatcaacttgaaaaagtggcaccgagtcggtgcgcatcattaaaactctctgccacgtaatagaggaaaaaaaaagctgccatcagtcggcgtggactgtagaacactgccaatgccggtcccaagcccggataaaagtggagggtacagtccacgcttttttttcaccttactttctccttggcatcattaaaactccctgccacgtaatagaggggctggaactccgacccgcgctaagcagcttggcgtaactagatct

SEQ ID NO: 161 - DNA, 350 bp, unidentified
  source 1..350: mol_type = unassigned DNA
ggccaaagcatgcatgcctccatgtattccactcagtttcagagctatgctggaaacagcatagcaagttgaaataaggctagtccgttatcaacttgaaaaagtggcaccgagtcggtgccctcgagtggaatacatggaaaaaaaaagctgccatcagtcggcgtggactgtagaacactgccaatgccggtcccaagcccggataaaagtggagggtacagtccacgcttttttttgcttgagacacctattttgtgcctgtattctagtgggcagcccatcccttgagtggaatacatggaggaagaaaagaacgtaacgctaagcagcttggcgtaactagatct

SEQ ID NO: 162 - DNA, 350 bp, unidentified
  source 1..350: mol_type = unassigned DNA
ggccaaagcatgcatgcctccatgtattccactcagtttcagagctatgctggaaacagcatagcaagttgaaataaggctagtccgttatcaacttgaaaaagtggcaccgagtcggtgccatccctcgagtggaatacatggaaaaaaaaagctgccatcagtcggcgtggactgtagaacactgccaatgccggtcccaagcccggataaaagtggagggtacagtccacgcttttttttgagacacctattttgtgcctgtattctagtgggcagcccatcccttgagtggaatacatggaggaagaaaagaactaaaagctaagcagcttggcgtaactagatct

SEQ ID NO: 163 - DNA, 350 bp, unidentified
  source 1..350: mol_type = unassigned DNA
ggccaaagcatgcatgcctccatgtattccactcagtttcagagctatgctggaaacagcatagcaagttgaaataaggctagtccgttatcaacttgaaaaagtggcaccgagtcggtgcgcccatccctcgagtggaatacatggaaaaaaaaagctgccatcagtcggcgtggactgtagaacactgccaatgccggtcccaagcccggataaaagtggagggtacagtccacgctttttttgacacctattttgtgcctgtattctagtgggcagcccatcccttgagtggaatacatggaggaagaaaagaataaaaagctaagcagcttggcgtaactagatct

SEQ ID NO: 164 - DNA, 350 bp, unidentified
  source 1..350: mol_type = unassigned DNA
ggccaaagcatgcatgcctccatgtattccactcagtttcagagctatgctggaaacagcatagcaagttgaaataaggctagtccgttatcaacttgaaaaagtggcaccgagtcggtgcgggcagcccatccctcgagtggaatacatggaaaaaaaaagctgccatcagtcggcgtggactgtagaacactgccaatgccggtcccaagcccggataaaagtggagggtacagtccacgctttttttctattttgtgcctgtattctagtgggcagcccatcccttgagtggaatacatggaggaagaaaagaatagagtgctaagcagcttggcgtaactagatct

SEQ ID NO: 165 - DNA, 350 bp, unidentified
  source 1..350: mol_type = unassigned DNA
ggccaaagcatgcatgctccatgtattccactcaagtttcagagctatgctggaaacagcatagcaagttgaaataaggctagtccgttatcaacttgaaaaagtggcaccgagtcggtgccctcgagtggaatacatgaaaaaaaaagctgccatcagtcggcgtggactgtagaacactgccaatgccggtcccaagcccggataaaagtggagggtacagtccacgctttttttcctgcttgagacacctattttgtgcctgtattctagtgggcagcccatcccttgagtggaatacatggaggaagaaaagatctcgtgctaagcagcttggcgtaactagatct

SEQ ID NO: 166 - DNA, 350 bp, unidentified
  source 1..350: mol_type = unassigned DNA
ggccaaagcatgcatgctccatgtattccactcaagtttcagagctatgctggaaacagcatagcaagttgaaataaggctagtccgttatcaacttgaaaaagtggcaccgagtcggtgccatccctcgagtggaatacatgaaaaaaaaagctgccatcagtcggcgtggactgtagaacactgccaatgccggtcccaagcccggataaaagtggagggtacagtccacgctttttttcttgagacacctattttgtgcctgtattctagtgggcagcccatcccttgagtggaatacatggaggaagaaaagatgtgcagctaagcagcttggcgtaactagatct

SEQ ID NO: 167 - DNA, 350 bp, unidentified
  source 1..350: mol_type = unassigned DNA
ggccaaagcatgcatgctccatgtattccactcaagtttcagagctatgctggaaacagcatagcaagttgaaataaggctagtccgttatcaacttgaaaaagtggcaccgagtcggtgcgcccatccctcgagtggaatacatgaaaaaaaaagctgccatcagtcggcgtggactgtagaacactgccaatgccggtcccaagcccggataaaagtggagggtacagtccacgctttttttgagacacctattttgtgcctgtattctagtgggcagcccatcccttgagtggaatacatggaggaagaaaagacacggcgctaagcagcttggcgtaactagatct

SEQ ID NO: 168 - DNA, 350 bp, unidentified
  source 1..350: mol_type = unassigned DNA
ggccaaagcatgcatgctccatgtattccactcaagtttcagagctatgctggaaacagcatagcaagttgaaataaggctagtccgttatcaacttgaaaaagtggcaccgagtcggtgcgggcagcccatccctcgagtggaatacatgaaaaaaaaagctgccatcagtcggcgtggactgtagaacactgccaatgccggtcccaagcccggataaaagtggagggtacagtccacgctttttttacctattttgtgcctgtattctagtgggcagcccatcccttgagtggaatacatggaggaagaaaagaggggcagctaagcagcttggcgtaactagatct

SEQ ID NO: 169 - DNA, 350 bp, unidentified
  source 1..350: mol_type = unassigned DNA
ggccaaagcatgcatgaggaaagcaggccagccacgtttcagagctatgctggaaacagcatagcaagttgaaataaggctagtccgttatcaacttgaaaaagtggcaccgagtcggtgctccgacctgtggctggcctgctttaaaaaaaaagctgccatcagtcggcgtggactgtagaacactgccaatgccggtcccaagcccggataaaagtggagggtacagtccacgcttttttttttcttcttttcatcccagcttgcactggtttccgcctccaacctgtggctggcctgctttcctctcgggatttgatatggctaagcagcttggcgtaactagatct

SEQ ID NO: 170 - DNA, 350 bp, unidentified
  source 1..350: mol_type = unassigned DNA
ggccaaagcatgcatgaggaaagcaggccagccacgtttcagagctatgctggaaacagcatagcaagttgaaataaggctagtccgttatcaacttgaaaaagtggcaccgagtcggtgcctggtttccgcctccgacctgtggctggcctgctttaaaaaaaaagctgccatcagtcggcgtggactgtagaacactgccaatgccggtcccaagcccggataaaagtggagggtacagtccacgctttttttatcccagcttgcactggtttccgcctccaacctgtggctggcctgctttcctctcgggattttcgagggctaagcagcttggcgtaactagatct

SEQ ID NO: 171 - DNA, 350 bp, unidentified
  source 1..350: mol_type = unassigned DNA
ggccaaagcatgcatgaagcaggccagccacaggtgtttcagagctatgctggaaacagcatagcaagttgaaataaggctagtccgttatcaacttgaaaaagtggcaccgagtcggtgctccgacctgtggctggcctgaaaaaaaaagctgccatcagtcggcgtggactgtagaacactgccaatgccggtcccaagcccggataaaagtggagggtacagtccacgctttttttgagttttctttcttcttttcatcccagcttgcactggtttccgcctccaacctgtggctggcctgctttcctctcgggtcaattgctaagcagcttggcgtaactagatct

SEQ ID NO: 172 - DNA, 350 bp, unidentified
  source 1..350: mol_type = unassigned DNA
ggccaaagcatgcatgaagcaggccagccacaggtgtttcagagctatgctggaaacagcatagcaagttgaaataaggctagtccgttatcaacttgaaaaagtggcaccgagtcggtgccgcctccgacctgtggctggcctgaaaaaaaaagctgccatcagtcggcgtggactgtagaacactgccaatgccggtcccaagcccggataaaagtggagggtacagtccacgcttttttttttctttcttcttttcatcccagcttgcactggtttccgcctccaacctgtggctggcctgctttcctctcgggtagtttgctaagcagcttggcgtaactagatct

SEQ ID NO: 173 - DNA, 350 bp, unidentified
  source 1..350: mol_type = unassigned DNA
ggccaaagcatgcatgaagcaggccagccacaggtgtttcagagctatgctggaaacagcatagcaagttgaaataaggctagtccgttatcaacttgaaaaagtggcaccgagtcggtgcctggtttccgcctccgacctgtggctggcctgaaaaaaaaagctgccatcagtcggcgtggactgtagaacactgccaatgccggtcccaagcccggataaaagtggagggtacagtccacgctttttttttcttttcatcccagcttgcactggtttccgcctccaacctgtggctggcctgctttcctctcgggtgtagcgctaagcagcttggcgtaactagatct

SEQ ID NO: 174 - DNA, 350 bp, unidentified
  source 1..350: mol_type = unassigned DNA
ggccaaagcatgcatgcaggccagccacaggttgggtttcagagctatgctggaaacagcatagcaagttgaaataaggctagtccgttatcaacttgaaaaagtggcaccgagtcggtgctccgacctgtggctggcaaaaaaaaagctgccatcagtcggcgtggactgtagaacactgccaatgccggtcccaagcccggataaaagtggagggtacagtccacgctttttttagctttgagttttctttcttcttttcatcccagcttgcactggtttccgcctccaacctgtggctggcctgctttcctctcgtacatgctaagcagcttggcgtaactagatct

SEQ ID NO: 175 - DNA, 350 bp, unidentified
  source 1..350: mol_type = unassigned DNA
ggccaaagcatgcatgcaggccagccacaggttgggtttcagagctatgctggaaacagcatagcaagttgaaataaggctagtccgttatcaacttgaaaaagtggcaccgagtcggtgcctggtttccgcctccgacctgtggctggcaaaaaaaaagctgccatcagtcggcgtggactgtagaacactgccaatgccggtcccaagcccggataaaagtggagggtacagtccacgcttttttttctttcttcttttcatcccagcttgcactggtttccgcctccaacctgtggctggcctgctttcctctcttttacgctaagcagcttggcgtaactagatct

SEQ ID NO: 176 - DNA, 350 bp, unidentified
  source 1..350: mol_type = unassigned DNA
ggccaaagcatgcatgcctccatgtattccactaggtttcagagctatgctggaaacagcatagcaagttgaaataaggctagtccgttatcaacttgaaaaagtggcaccgagtcggtgcctcgagtggaatacatggaaaaaaaaagctgccatcagtcggcgtggactgtagaacactgccaatgccggtcccaagcccggataaaagtggagggtacagtccacgctttttttctgcttgagacacctattttgtgcctgtattctagtgggcagcccatccctctagtggaatacatggaggaagaaaagaagtccatgctaagcagcttggcgtaactagatct

SEQ ID NO: 177 - DNA, 350 bp, unidentified
  source 1..350: mol_type = unassigned DNA
ggccaaagcatgcatgcctccatgtattccactaggtttcagagctatgctggaaacagcatagcaagttgaaataaggctagtccgttatcaacttgaaaaagtggcaccgagtcggtgcatccctcgagtggaatacatggaaaaaaaaagctgccatcagtcggcgtggactgtagaacactgccaatgccggtcccaagcccggataaaagtggagggtacagtccacgctttttttttgagacacctattttgtgcctgtattctagtgggcagcccatccctctagtggaatacatggaggaagaaaagaacgatttgctaagcagcttggcgtaactagatct

SEQ ID NO: 178 - DNA, 350 bp, unidentified
  source 1..350: mol_type = unassigned DNA
ggccaaagcatgcatgcctccatgtattccactaggtttcagagctatgctggaaacagcatagcaagttgaaataaggctagtccgttatcaacttgaaaaagtggcaccgagtcggtgccccatccctcgagtggaatacatggaaaaaaaaagctgccatcagtcggcgtggactgtagaacactgccaatgccggtcccaagcccggataaaagtggagggtacagtccacgctttttttagacacctattttgtgcctgtattctagtgggcagcccatccctctagtggaatacatggaggaagaaaagaattagtcgctaagcagcttggcgtaactagatct

SEQ ID NO: 179 - DNA, 350 bp, unidentified
  source 1..350: mol_type = unassigned DNA
ggccaaagcatgcatgcctccatgtattccactaggtttcagagctatgctggaaacagcatagcaagttgaaataaggctagtccgttatcaacttgaaaaagtggcaccgagtcggtgcggcagcccatccctcgagtggaatacatggaaaaaaaaagctgccatcagtcggcgtggactgtagaacactgccaatgccggtcccaagcccggataaaagtggagggtacagtccacgctttttttcctattttgtgcctgtattctagtgggcagcccatccctctagtggaatacatggaggaagaaaagaaggatgcgctaagcagcttggcgtaactagatct

SEQ ID NO: 180 - DNA, 350 bp, unidentified
  source 1..350: mol_type = unassigned DNA
ggccaaagcatgcatgctccatgtattccactagagtttcagagctatgctggaaacagcatagcaagttgaaataaggctagtccgttatcaacttgaaaaagtggcaccgagtcggtgcctcgagtggaatacatgaaaaaaaaagctgccatcagtcggcgtggactgtagaacactgccaatgccggtcccaagcccggataaaagtggagggtacagtccacgctttttttccctgcttgagacacctattttgtgcctgtattctagtgggcagcccatccctctagtggaatacatggaggaagaaaagaacccatgctaagcagcttggcgtaactagatct

SEQ ID NO: 181 - DNA, 350 bp, unidentified
  source 1..350: mol_type = unassigned DNA
ggccaaagcatgcatgctccatgtattccactagagtttcagagctatgctggaaacagcatagcaagttgaaataaggctagtccgttatcaacttgaaaaagtggcaccgagtcggtgcatccctcgagtggaatacatgaaaaaaaaagctgccatcagtcggcgtggactgtagaacactgccaatgccggtcccaagcccggataaaagtggagggtacagtccacgctttttttgcttgagacacctattttgtgcctgtattctagtgggcagcccatccctctagtggaatacatggaggaagaaaagactaccagctaagcagcttggcgtaactagatct

SEQ ID NO: 182 - DNA, 350 bp, unidentified
  source 1..350: mol_type = unassigned DNA
ggccaaagcatgcatgctccatgtattccactagagtttcagagctatgctggaaacagcatagcaagttgaaataaggctagtccgttatcaacttgaaaaagtggcaccgagtcggtgccccatccctcgagtggaatacatgaaaaaaaaagctgccatcagtcggcgtggactgtagaacactgccaatgccggtcccaagcccggataaaagtggagggtacagtccacgcttttttttgagacacctattttgtgcctgtattctagtgggcagcccatccctctagtggaatacatggaggaagaaaagagggatagctaagcagcttggcgtaactagatct

SEQ ID NO: 183 - DNA, 350 bp, unidentified
  source 1..350: mol_type = unassigned DNA
ggccaaagcatgcatgctccatgtattccactagagtttcagagctatgctggaaacagcatagcaagttgaaataaggctagtccgttatcaacttgaaaaagtggcaccgagtcggtgcggcagcccatccctcgagtggaatacatgaaaaaaaaagctgccatcagtcggcgtggactgtagaacactgccaatgccggtcccaagcccggataaaagtggagggtacagtccacgctttttttcacctattttgtgcctgtattctagtgggcagcccatccctctagtggaatacatggaggaagaaaagaaacgacgctaagcagcttggcgtaactagatct

SEQ ID NO: 184 - DNA, 350 bp, unidentified
  source 1..350: mol_type = unassigned DNA
ggccaaagcatgcatggggcagcccatccctctaggtttcagagctatgctggaaacagcatagcaagttgaaataaggctagtccgttatcaacttgaaaaagtggcaccgagtcggtgcactcgagggatgggctgaaaaaaaaagctgccatcagtcggcgtggactgtagaacactgccaatgccggtcccaagcccggataaaagtggagggtacagtccacgctttttttagagtcttgaacactgtgccccatgttttcttttcttcctccatgtattccactagagggatgggctgcccactagaataccaggccgctaagcagcttggcgtaactagatct

SEQ ID NO: 185 - DNA, 350 bp, unidentified
  source 1..350: mol_type = unassigned DNA
ggccaaagcatgcatggggcagcccatccctctaggtttcagagctatgctggaaacagcatagcaagttgaaataaggctagtccgttatcaacttgaaaaagtggcaccgagtcggtgcttccactcgagggatgggctgaaaaaaaaagctgccatcagtcggcgtggactgtagaacactgccaatgccggtcccaagcccggataaaagtggagggtacagtccacgcttttttttcttgaacactgtgccccatgttttcttttcttcctccatgtattccactagagggatgggctgcccactagaatacactggcgctaagcagcttggcgtaactagatct

SEQ ID NO: 186 - DNA, 350 bp, unidentified
  source 1..350: mol_type = unassigned DNA
ggccaaagcatgcatggggcagcccatccctctaggtttcagagctatgctggaaacagcatagcaagttgaaataaggctagtccgttatcaacttgaaaaagtggcaccgagtcggtgcgtattccactcgagggatgggctgaaaaaaaaagctgccatcagtcggcgtggactgtagaacactgccaatgccggtcccaagcccggataaaagtggagggtacagtccacgcttttttttgaacactgtgccccatgttttcttttcttcctccatgtattccactagagggatgggctgcccactagaatacagctacgctaagcagcttggcgtaactagatct

SEQ ID NO: 187 - DNA, 350 bp, unidentified
  source 1..350: mol_type = unassigned DNA
ggccaaagcatgcatggggcagcccatccctctaggtttcagagctatgctggaaacagcatagcaagttgaaataaggctagtccgttatcaacttgaaaaagtggcaccgagtcggtgctccatgtattccactcgagggatgggctgaaaaaaaaagctgccatcagtcggcgtggactgtagaacactgccaatgccggtcccaagcccggataaaagtggagggtacagtccacgctttttttactgtgccccatgttttcttttcttcctccatgtattccactagagggatgggctgcccactagaatacgcgcgggctaagcagcttggcgtaactagatct

SEQ ID NO: 188 - DNA, 350 bp, unidentified
  source 1..350: mol_type = unassigned DNA
ggccaaagcatgcatgccacccaagaaatcccaaggtttcagagctatgctggaaacagcatagcaagttgaaataaggctagtccgttatcaacttgaaaaagtggcaccgagtcggtgctctcgggatttcttgggaaaaaaaaagctgccatcagtcggcgtggactgtagaacactgccaatgccggtcccaagcccggataaaagtggagggtacagtccacgctttttttatcccagcttgcactggtttccgcctccgacctgtggctggcctgctttcctcttgggatttcttgggtggcctggccttcgccgaggctaagcagcttggcgtaactagatct

SEQ ID NO: 189 - DNA, 350 bp, unidentified
  source 1..350: mol_type = unassigned DNA
ggccaaagcatgcatgccacccaagaaatcccaaggtttcagagctatgctggaaacagcatagcaagttgaaataaggctagtccgttatcaacttgaaaaagtggcaccgagtcggtgcttcctctcgggatttcttgggaaaaaaaaagctgccatcagtcggcgtggactgtagaacactgccaatgccggtcccaagcccggataaaagtggagggtacagtccacgctttttttcagcttgcactggtttccgcctccgacctgtggctggcctgctttcctcttgggatttcttgggtggcctggccttcgttcgagctaagcagcttggcgtaactagatct

SEQ ID NO: 190 - DNA, 350 bp, unidentified
  source 1..350: mol_type = unassigned DNA
ggccaaagcatgcatgccacccaagaaatcccaaggtttcagagctatgctggaaacagcatagcaagttgaaataaggctagtccgttatcaacttgaaaaagtggcaccgagtcggtgcgctttcctctcgggatttcttgggaaaaaaaaagctgccatcagtcggcgtggactgtagaacactgccaatgccggtcccaagcccggataaaagtggagggtacagtccacgctttttttcttgcactggtttccgcctccgacctgtggctggcctgctttcctcttgggatttcttgggtggcctggccttcggctcggctaagcagcttggcgtaactagatct

SEQ ID NO: 191 - DNA, 350 bp, unidentified
  source 1..350: mol_type = unassigned DNA
ggccaaagcatgcatgccacccaagaaatcccaaggtttcagagctatgctggaaacagcatagcaagttgaaataaggctagtccgttatcaacttgaaaaagtggcaccgagtcggtgcggcctgctttcctctcgggatttcttgggaaaaaaaaagctgccatcagtcggcgtggactgtagaacactgccaatgccggtcccaagcccggataaaagtggagggtacagtccacgctttttttactggtttccgcctccgacctgtggctggcctgctttcctcttgggatttcttgggtggcctggccttctttttagctaagcagcttggcgtaactagatct

SEQ ID NO: 192 - DNA, 350 bp, unidentified
  source 1..350: mol_type = unassigned DNA
ggccaaagcatgcatggctggcctgctttcctcttgtttcagagctatgctggaaacagcatagcaagttgaaataaggctagtccgttatcaacttgaaaaagtggcaccgagtcggtgccccgagaggaaagcaggccaaaaaaaaagctgccatcagtcggcgtggactgtagaacactgccaatgccggtcccaagcccggataaaagtggagggtacagtccacgctttttttgtactgtgtgcagtggaagactcggaaggccaggccacccaagaaatcccaagaggaaagcaggccagccacaggtcgggaatacgctaagcagcttggcgtaactagatct

SEQ ID NO: 193 - DNA, 350 bp, unidentified
  source 1..350: mol_type = unassigned DNA
ggccaaagcatgcatggctggcctgctttcctcttgtttcagagctatgctggaaacagcatagcaagttgaaataaggctagtccgttatcaacttgaaaaagtggcaccgagtcggtgcaaatcccgagaggaaagcaggccaaaaaaaaagctgccatcagtcggcgtggactgtagaacactgccaatgccggtcccaagcccggataaaagtggagggtacagtccacgcttttttttgtgtgcagtggaagactcggaaggccaggccacccaagaaatcccaagaggaaagcaggccagccacaggtcggaaactagctaagcagcttggcgtaactagatct

SEQ ID NO: 194 - DNA, 350 bp, unidentified
  source 1..350: mol_type = unassigned DNA
ggccaaagcatgcatggaggacagtactcacggttgtttcagagctatgctggaaacagcatagcaagttgaaataaggctagtccgttatcaacttgaaaaagtggcaccgagtcggtgcatgtatacccccgaaccgtgagtactgtcaaaaaaaaagctgccatcagtcggcgtggactgtagaacactgccaatgccggtcccaagcccggataaaagtggagggtacagtccacgctttttttcagtacatcagacatggatccaagcccatggatacccccgaaccgtgagtactgtcctccagctaccagaccaaggctaagcagcttggcgtaactagatct

SEQ ID NO: 195 - DNA, 350 bp, unidentified
  source 1..350: mol_type = unassigned DNA
ggccaaagcatgcatggaggacagtactcacggttgtttcagagctatgctggaaacagcatagcaagttgaaataaggctagtccgttatcaacttgaaaaagtggcaccgagtcggtgcgcccatgtatacccccgaaccgtgagtactgtcaaaaaaaaagctgccatcagtcggcgtggactgtagaacactgccaatgccggtcccaagcccggataaaagtggagggtacagtccacgctttttttacatcagacatggatccaagcccatggatacccccgaaccgtgagtactgtcctccagctaccagccacatgctaagcagcttggcgtaactagatct

SEQ ID NO: 196 - DNA, 350 bp, unidentified
  source 1..350: mol_type = unassigned DNA
ggccaaagcatgcatggaggacagtactcacggttgtttcagagctatgctggaaacagcatagcaagttgaaataaggctagtccgttatcaacttgaaaaagtggcaccgagtcggtgccaagcccatgtatacccccgaaccgtgagtactgtcaaaaaaaaagctgccatcagtcggcgtggactgtagaacactgccaatgccggtcccaagcccggataaaagtggagggtacagtccacgcttttttttcagacatggatccaagcccatggatacccccgaaccgtgagtactgtcctccagctaccagtcctaggctaagcagcttggcgtaactagatct

SEQ ID NO: 197 - DNA, 350 bp, unidentified
  source 1..350: mol_type = unassigned DNA
ggccaaagcatgcatggaggacagtactcacggttgtttcagagctatgctggaaacagcatagcaagttgaaataaggctagtccgttatcaacttgaaaaagtggcaccgagtcggtgcggatccaagcccatgtatacccccgaaccgtgagtactgtcaaaaaaaaagctgccatcagtcggcgtggactgtagaacactgccaatgccggtcccaagcccggataaaagtggagggtacagtccacgctttttttcatggatccaagcccatggatacccccgaaccgtgagtactgtcctccagctaccagagaatcgctaagcagcttggcgtaactagatct

SEQ ID NO: 198 - DNA, 350 bp, unidentified
  source 1..350: mol_type = unassigned DNA
ggccaaagcatgcatgggacagtactcacggttcggtttcagagctatgctggaaacagcatagcaagttgaaataaggctagtccgttatcaacttgaaaaagtggcaccgagtcggtgcgcccatgtatacccccgaaccgtgagtactgaaaaaaaaagctgccatcagtcggcgtggactgtagaacactgccaatgccggtcccaagcccggataaaagtggagggtacagtccacgctttttttcagtacatcagacatggatccaagcccatggatacccccgaaccgtgagtactgtcctccagctaccaccgaagctaagcagcttggcgtaactagatct

SEQ ID NO: 199 - DNA, 350 bp, unidentified
  source 1..350: mol_type = unassigned DNA
ggccaaagcatgcatgggacagtactcacggttcggtttcagagctatgctggaaacagcatagcaagttgaaataaggctagtccgttatcaacttgaaaaagtggcaccgagtcggtgccaagcccatgtatacccccgaaccgtgagtactgaaaaaaaaagctgccatcagtcggcgtggactgtagaacactgccaatgccggtcccaagcccggataaaagtggagggtacagtccacgcttttttttacatcagacatggatccaagcccatggatacccccgaaccgtgagtactgtcctccagctaccagtgtcgctaagcagcttggcgtaactagatct

SEQ ID NO: 200 - DNA, 350 bp, unidentified
  source 1..350: mol_type = unassigned DNA
ggccaaagcatgcatgggacagtactcacggttcggtttcagagctatgctggaaacagcatagcaagttgaaataaggctagtccgttatcaacttgaaaaagtggcaccgagtcggtgcggatccaagcccatgtatacccccgaaccgtgagtactgaaaaaaaaagctgccatcagtcggcgtggactgtagaacactgccaatgccggtcccaagcccggataaaagtggagggtacagtccacgctttttttcagacatggatccaagcccatggatacccccgaaccgtgagtactgtcctccagctacccgttgagctaagcagcttggcgtaactagatct

SEQ ID NO: 201 - DNA, 350 bp, unidentified
  source 1..350: mol_type = unassigned DNA
ggccaaagcatgcatggacagtactcacggttcgggtttcagagctatgctggaaacagcatagcaagttgaaataaggctagtccgttatcaacttgaaaaagtggcaccgagtcggtgcatgtatacccccgaaccgtgagtactaaaaaaaaagctgccatcagtcggcgtggactgtagaacactgccaatgccggtcccaagcccggataaaagtggagggtacagtccacgcttttttttgcacacagtacatcagacatggatccaagcccatggatacccccgaaccgtgagtactgtcctccagctactctttcgctaagcagcttggcgtaactagatct

SEQ ID NO: 202 - DNA, 350 bp, unidentified
  source 1..350: mol_type = unassigned DNA
ggccaaagcatgcatggacagtactcacggttcgggtttcagagctatgctggaaacagcatagcaagttgaaataaggctagtccgttatcaacttgaaaaagtggcaccgagtcggtgcgcccatgtatacccccgaaccgtgagtactaaaaaaaaagctgccatcagtcggcgtggactgtagaacactgccaatgccggtcccaagcccggataaaagtggagggtacagtccacgctttttttcacagtacatcagacatggatccaagcccatggatacccccgaaccgtgagtactgtcctccagctaccctagggctaagcagcttggcgtaactagatct

SEQ ID NO: 203 - DNA, 350 bp, unidentified
  source 1..350: mol_type = unassigned DNA
ggccaaagcatgcatggacagtactcacggttcgggtttcagagctatgctggaaacagcatagcaagttgaaataaggctagtccgttatcaacttgaaaaagtggcaccgagtcggtgccaagcccatgtatacccccgaaccgtgagtactaaaaaaaaagctgccatcagtcggcgtggactgtagaacactgccaatgccggtcccaagcccggataaaagtggagggtacagtccacgctttttttagtacatcagacatggatccaagcccatggatacccccgaaccgtgagtactgtcctccagctacagtctagctaagcagcttggcgtaactagatct

SEQ ID NO: 204 - DNA, 350 bp, unidentified
  source 1..350: mol_type = unassigned DNA
ggccaaagcatgcatggacagtactcacggttcgggtttcagagctatgctggaaacagcatagcaagttgaaataaggctagtccgttatcaacttgaaaaagtggcaccgagtcggtgcggatccaagcccatgtatacccccgaaccgtgagtactaaaaaaaaagctgccatcagtcggcgtggactgtagaacactgccaatgccggtcccaagcccggataaaagtggagggtacagtccacgctttttttatcagacatggatccaagcccatggatacccccgaaccgtgagtactgtcctccagctacactacggctaagcagcttggcgtaactagatct

SEQ ID NO: 205 - DNA, 350 bp, unidentified
  source 1..350: mol_type = unassigned DNA
ggccaaagcatgcatggggaagcagcctgatgcccgtttcagagctatgctggaaacagcatagcaagttgaaataaggctagtccgttatcaacttgaaaaagtggcaccgagtcggtgcttctggggcatcaggctgcttaaaaaaaaagctgccatcagtcggcgtggactgtagaacactgccaatgccggtcccaagcccggataaaagtggagggtacagtccacgctttttttcccagccttgctccggatcagctccaaactgtcggggtttttcttccggggcatcaggctgcttcccgtgctggggaggaggagctaagcagcttggcgtaactagatct

SEQ ID NO: 206 - DNA, 350 bp, unidentified
  source 1..350: mol_type = unassigned DNA
ggccaaagcatgcatggggaagcagcctgatgcccgtttcagagctatgctggaaacagcatagcaagttgaaataaggctagtccgttatcaacttgaaaaagtggcaccgagtcggtgctttcttctggggcatcaggctgcttaaaaaaaaagctgccatcagtcggcgtggactgtagaacactgccaatgccggtcccaagcccggataaaagtggagggtacagtccacgctttttttgccttgctccggatcagctccaaactgtcggggtttttcttccggggcatcaggctgcttcccgtgctggggattccaggctaagcagcttggcgtaactagatct

SEQ ID NO: 207 - DNA, 350 bp, unidentified
  source 1..350: mol_type = unassigned DNA
ggccaaagcatgcatggggaagcagcctgatgcccgtttcagagctatgctggaaacagcatagcaagttgaaataaggctagtccgttatcaacttgaaaaagtggcaccgagtcggtgcgtttttcttctggggcatcaggctgcttaaaaaaaaagctgccatcagtcggcgtggactgtagaacactgccaatgccggtcccaagcccggataaaagtggagggtacagtccacgctttttttttgctccggatcagctccaaactgtcggggtttttcttccggggcatcaggctgcttcccgtgctggggaagttcggctaagcagcttggcgtaactagatct

SEQ ID NO: 208 - DNA, 350 bp, unidentified
  source 1..350: mol_type = unassigned DNA
ggccaaagcatgcatggggaagcagcctgatgcccgtttcagagctatgctggaaacagcatagcaagttgaaataaggctagtccgttatcaacttgaaaaagtggcaccgagtcggtgctcggggtttttcttctggggcatcaggctgcttaaaaaaaaagctgccatcagtcggcgtggactgtagaacactgccaatgccggtcccaagcccggataaaagtggagggtacagtccacgctttttttccggatcagctccaaactgtcggggtttttcttccggggcatcaggctgcttcccgtgctggggaggcacagctaagcagcttggcgtaactagatct

SEQ ID NO: 209 - DNA, 350 bp, unidentified
  source 1..350: mol_type = unassigned DNA
ggccaaagcatgcatgactgtcggggtttttcttcgtttcagagctatgctggaaacagcatagcaagttgaaataaggctagtccgttatcaacttgaaaaagtggcaccgagtcggtgccccagaagaaaaaccccgacaaaaaaaaagctgccatcagtcggcgtggactgtagaacactgccaatgccggtcccaagcccggataaaagtggagggtacagtccacgcttttttttcagcgccagcacctctgtccccagcacgggaagcagcctgatgccccggaagaaaaaccccgacagtttggagctgaaaaactgctaagcagcttggcgtaactagatct

SEQ ID NO: 210 - DNA, 350 bp, unidentified
  source 1..350: mol_type = unassigned DNA
ggccaaagcatgcatgactgtcggggtttttcttcgtttcagagctatgctggaaacagcatagcaagttgaaataaggctagtccgttatcaacttgaaaaagtggcaccgagtcggtgcatgccccagaagaaaaaccccgacaaaaaaaaagctgccatcagtcggcgtggactgtagaacactgccaatgccggtcccaagcccggataaaagtggagggtacagtccacgctttttttcgccagcacctctgtccccagcacgggaagcagcctgatgccccggaagaaaaaccccgacagtttggagctgactggctgctaagcagcttggcgtaactagatct

SEQ ID NO: 211 - DNA, 350 bp, unidentified
  source 1..350: mol_type = unassigned DNA
ggccaaagcatgcatgactgtcggggtttttcttcgtttcagagctatgctggaaacagcatagcaagttgaaataaggctagtccgttatcaacttgaaaaagtggcaccgagtcggtgcctgatgccccagaagaaaaaccccgacaaaaaaaaagctgccatcagtcggcgtggactgtagaacactgccaatgccggtcccaagcccggataaaagtggagggtacagtccacgctttttttcagcacctctgtccccagcacgggaagcagcctgatgccccggaagaaaaaccccgacagtttggagctgatgcacggctaagcagcttggcgtaactagatct

SEQ ID NO: 212 - DNA, 350 bp, unidentified
  source 1..350: mol_type = unassigned DNA
ggccaaagcatgcatgactgtcggggtttttcttcgtttcagagctatgctggaaacagcatagcaagttgaaataaggctagtccgttatcaacttgaaaaagtggcaccgagtcggtgcgcagcctgatgccccagaagaaaaaccccgacaaaaaaaaagctgccatcagtcggcgtggactgtagaacactgccaatgccggtcccaagcccggataaaagtggagggtacagtccacgctttttttcctctgtccccagcacgggaagcagcctgatgccccggaagaaaaaccccgacagtttggagctgaacgagggctaagcagcttggcgtaactagatct

SEQ ID NO: 213 - DNA, 350 bp, unidentified
  source 1..350: mol_type = unassigned DNA
ggccaaagcatgcatgctgtcggggtttttcttccgtttcagagctatgctggaaacagcatagcaagttgaaataaggctagtccgttatcaacttgaaaaagtggcaccgagtcggtgccccagaagaaaaaccccgaaaaaaaaaagctgccatcagtcggcgtggactgtagaacactgccaatgccggtcccaagcccggataaaagtggagggtacagtccacgctttttttcttcagcgccagcacctctgtccccagcacgggaagcagcctgatgccccggaagaaaaaccccgacagtttggagctggaaatggctaagcagcttggcgtaactagatct

SEQ ID NO: 214 - DNA, 350 bp, unidentified
  source 1..350: mol_type = unassigned DNA
ggccaaagcatgcatgctgtcggggtttttcttccgtttcagagctatgctggaaacagcatagcaagttgaaataaggctagtccgttatcaacttgaaaaagtggcaccgagtcggtgcatgccccagaagaaaaaccccgaaaaaaaaaagctgccatcagtcggcgtggactgtagaacactgccaatgccggtcccaagcccggataaaagtggagggtacagtccacgctttttttagcgccagcacctctgtccccagcacgggaagcagcctgatgccccggaagaaaaaccccgacagtttggagctgcggattgctaagcagcttggcgtaactagatct

SEQ ID NO: 215 - DNA, 350 bp, unidentified
  source 1..350: mol_type = unassigned DNA
ggccaaagcatgcatgctgtcggggtttttcttccgtttcagagctatgctggaaacagcatagcaagttgaaataaggctagtccgttatcaacttgaaaaagtggcaccgagtcggtgcctgatgccccagaagaaaaaccccgaaaaaaaaaagctgccatcagtcggcgtggactgtagaacactgccaatgccggtcccaagcccggataaaagtggagggtacagtccacgctttttttgccagcacctctgtccccagcacgggaagcagcctgatgccccggaagaaaaaccccgacagtttggagctgactggagctaagcagcttggcgtaactagatct

SEQ ID NO: 216 - DNA, 350 bp, unidentified
  source 1..350: mol_type = unassigned DNA
ggccaaagcatgcatgctgtcggggtttttcttccgtttcagagctatgctggaaacagcatagcaagttgaaataaggctagtccgttatcaacttgaaaaagtggcaccgagtcggtgcgcagcctgatgccccagaagaaaaaccccgaaaaaaaaaagctgccatcagtcggcgtggactgtagaacactgccaatgccggtcccaagcccggataaaagtggagggtacagtccacgctttttttcacctctgtccccagcacgggaagcagcctgatgccccggaagaaaaaccccgacagtttggagctgagggaggctaagcagcttggcgtaactagatct

SEQ ID NO: 217 - DNA, 350 bp, unidentified
  source 1..350: mol_type = unassigned DNA
ggccaaagcatgcatgcatacctgtaattcaccaagtttcagagctatgctggaaacagcatagcaagttgaaataaggctagtccgttatcaacttgaaaaagtggcaccgagtcggtgcctcctgtcatcctttggtgaattacaggtaaaaaaaaagctgccatcagtcggcgtggactgtagaacactgccaatgccggtcccaagcccggataaaagtggagggtacagtccacgctttttttgactccataaaggcatatggtgctgggctcgtgtcatcctttggtgaattacaggtatgaccttcacagggagaagctaagcagcttggcgtaactagatct

SEQ ID NO: 218 - DNA, 350 bp, unidentified
  source 1..350: mol_type = unassigned DNA
ggccaaagcatgcatgcatacctgtaattcaccaagtttcagagctatgctggaaacagcatagcaagttgaaataaggctagtccgttatcaacttgaaaaagtggcaccgagtcggtgctgggctcctgtcatcctttggtgaattacaggtaaaaaaaaagctgccatcagtcggcgtggactgtagaacactgccaatgccggtcccaagcccggataaaagtggagggtacagtccacgctttttttccataaaggcatatggtgctgggctcgtgtcatcctttggtgaattacaggtatgaccttcacagcccagagctaagcagcttggcgtaactagatct

SEQ ID NO: 219 - DNA, 350 bp, unidentified
  source 1..350: mol_type = unassigned DNA
ggccaaagcatgcatgcatacctgtaattcaccaagtttcagagctatgctggaaacagcatagcaagttgaaataaggctagtccgttatcaacttgaaaaagtggcaccgagtcggtgctgctgggctcctgtcatcctttggtgaattacaggtaaaaaaaaagctgccatcagtcggcgtggactgtagaacactgccaatgccggtcccaagcccggataaaagtggagggtacagtccacgcttttttttaaaggcatatggtgctgggctcgtgtcatcctttggtgaattacaggtatgaccttcacaggaagaagctaagcagcttggcgtaactagatct

SEQ ID NO: 220 - DNA, 350 bp, unidentified
  source 1..350: mol_type = unassigned DNA
ggccaaagcatgcatgcatacctgtaattcaccaagtttcagagctatgctggaaacagcatagcaagttgaaataaggctagtccgttatcaacttgaaaaagtggcaccgagtcggtgctatggtgctgggctcctgtcatcctttggtgaattacaggtaaaaaaaaagctgccatcagtcggcgtggactgtagaacactgccaatgccggtcccaagcccggataaaagtggagggtacagtccacgctttttttgcatatggtgctgggctcgtgtcatcctttggtgaattacaggtatgaccttcacaggttgtcgctaagcagcttggcgtaactagatct

SEQ ID NO: 221 - DNA, 350 bp, unidentified
  source 1..350: mol_type = unassigned DNA
ggccaaagcatgcatgccataaaggcatatggtgcgtttcagagctatgctggaaacagcatagcaagttgaaataaggctagtccgttatcaacttgaaaaagtggcaccgagtcggtgcgatgacaggagcccagcaccatatgcctttaaaaaaaaaagctgccatcagtcggcgtggactgtagaacactgccaatgccggtcccaagcccggataaaagtggagggtacagtccacgctttttttgtcatacctgtaattcaccaaaggatgacacgagcccagcaccatatgcctttatggagtctccttggaataggctaagcagcttggcgtaactagatct

SEQ ID NO: 222 - DNA, 350 bp, unidentified
  source 1..350: mol_type = unassigned DNA
ggccaaagcatgcatgccataaaggcatatggtgcgtttcagagctatgctggaaacagcatagcaagttgaaataaggctagtccgttatcaacttgaaaaagtggcaccgagtcggtgcaaggatgacaggagcccagcaccatatgcctttaaaaaaaaaagctgccatcagtcggcgtggactgtagaacactgccaatgccggtcccaagcccggataaaagtggagggtacagtccacgctttttttatacctgtaattcaccaaaggatgacacgagcccagcaccatatgcctttatggagtctccttgcgggcagctaagcagcttggcgtaactagatct

SEQ ID NO: 223 - DNA, 350 bp, unidentified
  source 1..350: mol_type = unassigned DNA
ggccaaagcatgcatgccataaaggcatatggtgcgtttcagagctatgctggaaacagcatagcaagttgaaataaggctagtccgttatcaacttgaaaaagtggcaccgagtcggtgccaccaaaggatgacaggagcccagcaccatatgcctttaaaaaaaaaagctgccatcagtcggcgtggactgtagaacactgccaatgccggtcccaagcccggataaaagtggagggtacagtccacgcttttttttgtaattcaccaaaggatgacacgagcccagcaccatatgcctttatggagtctccttggttaaggctaagcagcttggcgtaactagatct

SEQ ID NO: 224 - DNA, 350 bp, unidentified
  source 1..350: mol_type = unassigned DNA
ggccaaagcatgcatgcataaaggcatatggtgctgtttcagagctatgctggaaacagcatagcaagttgaaataaggctagtccgttatcaacttgaaaaagtggcaccgagtcggtgcacaggagcccagcaccatatgcctttaaaaaaaaagctgccatcagtcggcgtggactgtagaacactgccaatgccggtcccaagcccggataaaagtggagggtacagtccacgctttttttgtgaaggtcatacctgtaattcaccaaaggatgacacgagcccagcaccatatgcctttatggagtctccttggaccagctaagcagcttggcgtaactagatct

SEQ ID NO: 225 - DNA, 350 bp, unidentified
  source 1..350: mol_type = unassigned DNA
ggccaaagcatgcatgcataaaggcatatggtgctgtttcagagctatgctggaaacagcatagcaagttgaaataaggctagtccgttatcaacttgaaaaagtggcaccgagtcggtgcgatgacaggagcccagcaccatatgcctttaaaaaaaaagctgccatcagtcggcgtggactgtagaacactgccaatgccggtcccaagcccggataaaagtggagggtacagtccacgctttttttaggtcatacctgtaattcaccaaaggatgacacgagcccagcaccatatgcctttatggagtctcctttaccgagctaagcagcttggcgtaactagatct

SEQ ID NO: 226 - DNA, 350 bp, unidentified
  source 1..350: mol_type = unassigned DNA
ggccaaagcatgcatgcataaaggcatatggtgctgtttcagagctatgctggaaacagcatagcaagttgaaataaggctagtccgttatcaacttgaaaaagtggcaccgagtcggtgcaaggatgacaggagcccagcaccatatgcctttaaaaaaaaagctgccatcagtcggcgtggactgtagaacactgccaatgccggtcccaagcccggataaaagtggagggtacagtccacgcttttttttcatacctgtaattcaccaaaggatgacacgagcccagcaccatatgcctttatggagtctcctttacgctgctaagcagcttggcgtaactagatct

SEQ ID NO: 227 - DNA, 350 bp, unidentified
  source 1..350: mol_type = unassigned DNA
ggccaaagcatgcatgcataaaggcatatggtgctgtttcagagctatgctggaaacagcatagcaagttgaaataaggctagtccgttatcaacttgaaaaagtggcaccgagtcggtgccaccaaaggatgacaggagcccagcaccatatgcctttaaaaaaaaagctgccatcagtcggcgtggactgtagaacactgccaatgccggtcccaagcccggataaaagtggagggtacagtccacgctttttttcctgtaattcaccaaaggatgacacgagcccagcaccatatgcctttatggagtctccttaatattgctaagcagcttggcgtaactagatct

SEQ ID NO: 228 - DNA, 350 bp, unidentified
  source 1..350: mol_type = unassigned DNA
ggccaaagcatgcatgaggaaagcaggccagccacgtttcagagctatgctggaaacagcatagcaagttgaaataaggctagtccgttatcaacttgaaaaagtggcaccgagtcggtgcctccgacctgtggctggcctgctttaaaaaaaaagctgccatcagtcggcgtggactgtagaacactgccaatgccggtcccaagcccggataaaagtggagggtacagtccacgctttttttttcttcttttcatcccagcttgcactggtttccgcctctgacctgtggctggcctgctttcctctcgggatttcagatcgctaagcagcttggcgtaactagatct

SEQ ID NO: 229 - DNA, 350 bp, unidentified
  source 1..350: mol_type = unassigned DNA
ggccaaagcatgcatgaggaaagcaggccagccacgtttcagagctatgctggaaacagcatagcaagttgaaataaggctagtccgttatcaacttgaaaaagtggcaccgagtcggtgcccgcctccgacctgtggctggcctgctttaaaaaaaaagctgccatcagtcggcgtggactgtagaacactgccaatgccggtcccaagcccggataaaagtggagggtacagtccacgcttttttttcttttcatcccagcttgcactggtttccgcctctgacctgtggctggcctgctttcctctcgggatttgaactcgctaagcagcttggcgtaactagatct

SEQ ID NO: 230 - DNA, 350 bp, unidentified
  source 1..350: mol_type = unassigned DNA
ggccaaagcatgcatgaggaaagcaggccagccacgtttcagagctatgctggaaacagcatagcaagttgaaataaggctagtccgttatcaacttgaaaaagtggcaccgagtcggtgctttccgcctccgacctgtggctggcctgctttaaaaaaaaagctgccatcagtcggcgtggactgtagaacactgccaatgccggtcccaagcccggataaaagtggagggtacagtccacgcttttttttttcatcccagcttgcactggtttccgcctctgacctgtggctggcctgctttcctctcgggatttttctgtgctaagcagcttggcgtaactagatct

SEQ ID NO: 231 - DNA, 350 bp, unidentified
  source 1..350: mol_type = unassigned DNA
ggccaaagcatgcatgaggaaagcaggccagccacgtttcagagctatgctggaaacagcatagcaagttgaaataaggctagtccgttatcaacttgaaaaagtggcaccgagtcggtgcactggtttccgcctccgacctgtggctggcctgctttaaaaaaaaagctgccatcagtcggcgtggactgtagaacactgccaatgccggtcccaagcccggataaaagtggagggtacagtccacgcttttttttcccagcttgcactggtttccgcctctgacctgtggctggcctgctttcctctcgggatttgctcgcgctaagcagcttggcgtaactagatct

SEQ ID NO: 232 - DNA, 350 bp, unidentified
  source 1..350: mol_type = unassigned DNA
ggccaaagcatgcatgcaggccagccacaggtcaggtttcagagctatgctggaaacagcatagcaagttgaaataaggctagtccgttatcaacttgaaaaagtggcaccgagtcggtgcctccgacctgtggctggcaaaaaaaaagctgccatcagtcggcgtggactgtagaacactgccaatgccggtcccaagcccggataaaagtggagggtacagtccacgctttttttgctttgagttttctttcttcttttcatcccagcttgcactggtttccgcctctgacctgtggctggcctgctttcctctctactgggctaagcagcttggcgtaactagatct

SEQ ID NO: 233 - DNA, 350 bp, unidentified
  source 1..350: mol_type = unassigned DNA
ggccaaagcatgcatgcaggccagccacaggtcaggtttcagagctatgctggaaacagcatagcaagttgaaataaggctagtccgttatcaacttgaaaaagtggcaccgagtcggtgcccgcctccgacctgtggctggcaaaaaaaaagctgccatcagtcggcgtggactgtagaacactgccaatgccggtcccaagcccggataaaagtggagggtacagtccacgcttttttttgagttttctttcttcttttcatcccagcttgcactggtttccgcctctgacctgtggctggcctgctttcctctccttcacgctaagcagcttggcgtaactagatct

SEQ ID NO: 234 - DNA, 350 bp, unidentified
  source 1..350: mol_type = unassigned DNA
ggccaaagcatgcatgcaggccagccacaggtcaggtttcagagctatgctggaaacagcatagcaagttgaaataaggctagtccgttatcaacttgaaaaagtggcaccgagtcggtgctttccgcctccgacctgtggctggcaaaaaaaaagctgccatcagtcggcgtggactgtagaacactgccaatgccggtcccaagcccggataaaagtggagggtacagtccacgctttttttgttttctttcttcttttcatcccagcttgcactggtttccgcctctgacctgtggctggcctgctttcctctctcttgcgctaagcagcttggcgtaactagatct

SEQ ID NO: 235 - DNA, 350 bp, unidentified
  source 1..350: mol_type = unassigned DNA
ggccaaagcatgcatgcaggccagccacaggtcaggtttcagagctatgctggaaacagcatagcaagttgaaataaggctagtccgttatcaacttgaaaaagtggcaccgagtcggtgcactggtttccgcctccgacctgtggctggcaaaaaaaaagctgccatcagtcggcgtggactgtagaacactgccaatgccggtcccaagcccggataaaagtggagggtacagtccacgctttttttctttcttcttttcatcccagcttgcactggtttccgcctctgacctgtggctggcctgctttcctctccaaatagctaagcagcttggcgtaactagatct

SEQ ID NO: 236 - DNA, 350 bp, unidentified
  source 1..350: mol_type = unassigned DNA
ggccaaagcatgcatgacggttcgggggtatacatgtttcagagctatgctggaaacagcatagcaagttgaaataaggctagtccgttatcaacttgaaaaagtggcaccgagtcggtgccatggatccaagcccatgtatacccccgaacaaaaaaaaagctgccatcagtcggcgtggactgtagaacactgccaatgccggtcccaagcccggataaaagtggagggtacagtccacgcttttttttccactgcacacagtacatcagacattgatccaagcccatgtatacccccgaaccgtgagtactgtctgtggcgctaagcagcttggcgtaactagatct

SEQ ID NO: 237 - DNA, 350 bp, unidentified
  source 1..350: mol_type = unassigned DNA
ggccaaagcatgcatgacggttcgggggtatacatgtttcagagctatgctggaaacagcatagcaagttgaaataaggctagtccgttatcaacttgaaaaagtggcaccgagtcggtgccagacatggatccaagcccatgtatacccccgaacaaaaaaaaagctgccatcagtcggcgtggactgtagaacactgccaatgccggtcccaagcccggataaaagtggagggtacagtccacgctttttttctgcacacagtacatcagacattgatccaagcccatgtatacccccgaaccgtgagtactgtcacaactgctaagcagcttggcgtaactagatct

SEQ ID NO: 238 - DNA, 350 bp, unidentified
  source 1..350: mol_type = unassigned DNA
ggccaaagcatgcatgacggttcgggggtatacatgtttcagagctatgctggaaacagcatagcaagttgaaataaggctagtccgttatcaacttgaaaaagtggcaccgagtcggtgccatcagacatggatccaagcccatgtatacccccgaacaaaaaaaaagctgccatcagtcggcgtggactgtagaacactgccaatgccggtcccaagcccggataaaagtggagggtacagtccacgctttttttcacacagtacatcagacattgatccaagcccatgtatacccccgaaccgtgagtactgtcggtacagctaagcagcttggcgtaactagatct

SEQ ID NO: 239 - DNA, 350 bp, unidentified
  source 1..350: mol_type = unassigned DNA
ggccaaagcatgcatgtcgggggtatacatgggctgtttcagagctatgctggaaacagcatagcaagttgaaataaggctagtccgttatcaacttgaaaaagtggcaccgagtcggtgccatggatccaagcccatgtataccccaaaaaaaaagctgccatcagtcggcgtggactgtagaacactgccaatgccggtcccaagcccggataaaagtggagggtacagtccacgctttttttttccgagtcttccactgcacacagtacatcagacattgatccaagcccatgtatacccccgaaccgtgagtagatcgagctaagcagcttggcgtaactagatct

SEQ ID NO: 240 - DNA, 350 bp, unidentified
  source 1..350: mol_type = unassigned DNA
ggccaaagcatgcatgaggaaagcaggccagccacgtttcagagctatgctggaaacagcatagcaagttgaaataaggctagtccgttatcaacttgaaaaagtggcaccgagtcggtgctggtttccgcctccgacctgtggctggcctgctttaaaaaaaaagctgccatcagtcggcgtggactgtagaacactgccaatgccggtcccaagcccggataaaagtggagggtacagtccacgctttttttcatcccagcttgcactggtttctgcctccgacctgtggctggcctgctttcctctcgggatttgttgctgctaagcagcttggcgtaactagatct

SEQ ID NO: 241 - DNA, 350 bp, unidentified
  source 1..350: mol_type = unassigned DNA
ggccaaagcatgcatgaagcaggccagccacaggtgtttcagagctatgctggaaacagcatagcaagttgaaataaggctagtccgttatcaacttgaaaaagtggcaccgagtcggtgctggtttccgcctccgacctgtggctggcctgaaaaaaaaagctgccatcagtcggcgtggactgtagaacactgccaatgccggtcccaagcccggataaaagtggagggtacagtccacgctttttttcttcttttcatcccagcttgcactggtttctgcctccgacctgtggctggcctgctttcctctcgggaaatcagctaagcagcttggcgtaactagatct

SEQ ID NO: 242 - DNA, 350 bp, unidentified
  source 1..350: mol_type = unassigned DNA
ggccaaagcatgcatgaagcaggccagccacaggtgtttcagagctatgctggaaacagcatagcaagttgaaataaggctagtccgttatcaacttgaaaaagtggcaccgagtcggtgccactggtttccgcctccgacctgtggctggcctgaaaaaaaaagctgccatcagtcggcgtggactgtagaacactgccaatgccggtcccaagcccggataaaagtggagggtacagtccacgctttttttcttttcatcccagcttgcactggtttctgcctccgacctgtggctggcctgctttcctctcgggcaacccgctaagcagcttggcgtaactagatct

SEQ ID NO: 243 - DNA, 350 bp, unidentified
  source 1..350: mol_type = unassigned DNA
ggccaaagcatgcatgaagcaggccagccacaggtgtttcagagctatgctggaaacagcatagcaagttgaaataaggctagtccgttatcaacttgaaaaagtggcaccgagtcggtgcgcttgcactggtttccgcctccgacctgtggctggcctgaaaaaaaaagctgccatcagtcggcgtggactgtagaacactgccaatgccggtcccaagcccggataaaagtggagggtacagtccacgctttttttcatcccagcttgcactggtttctgcctccgacctgtggctggcctgctttcctctcgggtggacggctaagcagcttggcgtaactagatct

SEQ ID NO: 244 - DNA, 350 bp, unidentified
  source 1..350: mol_type = unassigned DNA
ggccaaagcatgcatgttttcatcccagcttgcacgtttcagagctatgctggaaacagcatagcaagttgaaataaggctagtccgttatcaacttgaaaaagtggcaccgagtcggtgcggtcggaggcggaaaccagtgcaagctgggatgaaaaaaaaaagctgccatcagtcggcgtggactgtagaacactgccaatgccggtcccaagcccggataaaagtggagggtacagtccacgctttttttaaagcaggccagccacaggtcggaggcagaaaccagtgcaagctgggatgaaaagaagaaagaacctctcgctaagcagcttggcgtaactagatct

SEQ ID NO: 245 - DNA, 350 bp, unidentified
  source 1..350: mol_type = unassigned DNA
ggccaaagcatgcatgttttcatcccagcttgcacgtttcagagctatgctggaaacagcatagcaagttgaaataaggctagtccgttatcaacttgaaaaagtggcaccgagtcggtgcccacaggtcggaggcggaaaccagtgcaagctgggatgaaaaaaaaaagctgccatcagtcggcgtggactgtagaacactgccaatgccggtcccaagcccggataaaagtggagggtacagtccacgctttttttaggccagccacaggtcggaggcagaaaccagtgcaagctgggatgaaaagaagaaagaaacccaagctaagcagcttggcgtaactagatct

SEQ ID NO: 246 - DNA, 350 bp, unidentified
  source 1..350: mol_type = unassigned DNA
ggccaaagcatgcatgctgaaaaaatctcatcctagtttcagagctatgctggaaacagcatagcaagttgaaataaggctagtccgttatcaacttgaaaaagtggcaccgagtcggtgccaccctgtgagtccatggcccgtaggatgagattttttaaaaaaaaagctgccatcagtcggcgtggactgtagaacactgccaatgccggtcccaagcccggataaaagtggagggtacagtccacgctttttttgatgctgaccaccctgtgattccatggcccgtaggatgagattttttcagtgcctctcctgcaccggctaagcagcttggcgtaactagatct

SEQ ID NO: 247 - DNA, 350 bp, unidentified
  source 1..350: mol_type = unassigned DNA
ggccaaagcatgcatgctgaaaaaatctcatcctagtttcagagctatgctggaaacagcatagcaagttgaaataaggctagtccgttatcaacttgaaaaagtggcaccgagtcggtgcctgaccaccctgtgagtccatggcccgtaggatgagattttttaaaaaaaaagctgccatcagtcggcgtggactgtagaacactgccaatgccggtcccaagcccggataaaagtggagggtacagtccacgcttttttttgaccaccctgtgattccatggcccgtaggatgagattttttcagtgcctctcctgccgaagctaagcagcttggcgtaactagatct

SEQ ID NO: 248 - DNA, 350 bp, unidentified
  source 1..350: mol_type = unassigned DNA
ggccaaagcatgcatgtgaaaaaatctcatcctacgtttcagagctatgctggaaacagcatagcaagttgaaataaggctagtccgttatcaacttgaaaaagtggcaccgagtcggtgctgagtccatggcccgtaggatgagatttttaaaaaaaaagctgccatcagtcggcgtggactgtagaacactgccaatgccggtcccaagcccggataaaagtggagggtacagtccacgctttttttgcggaactggatgctgaccaccctgtgattccatggcccgtaggatgagattttttcagtgcctctccccgcaagctaagcagcttggcgtaactagatct

SEQ ID NO: 249 - DNA, 350 bp, unidentified
  source 1..350: mol_type = unassigned DNA
ggccaaagcatgcatgtgaaaaaatctcatcctacgtttcagagctatgctggaaacagcatagcaagttgaaataaggctagtccgttatcaacttgaaaaagtggcaccgagtcggtgccctgtgagtccatggcccgtaggatgagatttttaaaaaaaaagctgccatcagtcggcgtggactgtagaacactgccaatgccggtcccaagcccggataaaagtggagggtacagtccacgctttttttaactggatgctgaccaccctgtgattccatggcccgtaggatgagattttttcagtgcctctccttctctgctaagcagcttggcgtaactagatct

SEQ ID NO: 250 - DNA, 350 bp, unidentified
  source 1..350: mol_type = unassigned DNA
ggccaaagcatgcatgtgaaaaaatctcatcctacgtttcagagctatgctggaaacagcatagcaagttgaaataaggctagtccgttatcaacttgaaaaagtggcaccgagtcggtgccaccctgtgagtccatggcccgtaggatgagatttttaaaaaaaaagctgccatcagtcggcgtggactgtagaacactgccaatgccggtcccaagcccggataaaagtggagggtacagtccacgcttttttttggatgctgaccaccctgtgattccatggcccgtaggatgagattttttcagtgcctctccttaaaggctaagcagcttggcgtaactagatct

SEQ ID NO: 251 - DNA, 350 bp, unidentified
  source 1..350: mol_type = unassigned DNA
ggccaaagcatgcatgtgaaaaaatctcatcctacgtttcagagctatgctggaaacagcatagcaagttgaaataaggctagtccgttatcaacttgaaaaagtggcaccgagtcggtgcctgaccaccctgtgagtccatggcccgtaggatgagatttttaaaaaaaaagctgccatcagtcggcgtggactgtagaacactgccaatgccggtcccaagcccggataaaagtggagggtacagtccacgctttttttgctgaccaccctgtgattccatggcccgtaggatgagattttttcagtgcctctccacccacgctaagcagcttggcgtaactagatct

SEQ ID NO: 252 - DNA, 350 bp, unidentified
  source 1..350: mol_type = unassigned DNA
ggccaaagcatgcatgaatctcatcctacgggccagtttcagagctatgctggaaacagcatagcaagttgaaataaggctagtccgttatcaacttgaaaaagtggcaccgagtcggtgctgagtccatggcccgtaggatgagaaaaaaaaagctgccatcagtcggcgtggactgtagaacactgccaatgccggtcccaagcccggataaaagtggagggtacagtccacgctttttttctcagctatggagcggaactggatgctgaccaccctgtgattccatggcccgtaggatgagattttttcagtgcagtttggctaagcagcttggcgtaactagatct

SEQ ID NO: 253 - DNA, 350 bp, unidentified
  source 1..350: mol_type = unassigned DNA
ggccaaagcatgcatgaatctcatcctacgggccagtttcagagctatgctggaaacagcatagcaagttgaaataaggctagtccgttatcaacttgaaaaagtggcaccgagtcggtgccctgtgagtccatggcccgtaggatgagaaaaaaaaagctgccatcagtcggcgtggactgtagaacactgccaatgccggtcccaagcccggataaaagtggagggtacagtccacgctttttttgctatggagcggaactggatgctgaccaccctgtgattccatggcccgtaggatgagattttttcagtgcaagtaggctaagcagcttggcgtaactagatct

SEQ ID NO: 254 - DNA, 350 bp, unidentified
  source 1..350: mol_type = unassigned DNA
ggccaaagcatgcatgaatctcatcctacgggccagtttcagagctatgctggaaacagcatagcaagttgaaataaggctagtccgttatcaacttgaaaaagtggcaccgagtcggtgccaccctgtgagtccatggcccgtaggatgagaaaaaaaaagctgccatcagtcggcgtggactgtagaacactgccaatgccggtcccaagcccggataaaagtggagggtacagtccacgctttttttatggagcggaactggatgctgaccaccctgtgattccatggcccgtaggatgagattttttcagtgctcaagagctaagcagcttggcgtaactagatct

SEQ ID NO: 255 - DNA, 350 bp, unidentified
  source 1..350: mol_type = unassigned DNA
ggccaaagcatgcatgaatctcatcctacgggccagtttcagagctatgctggaaacagcatagcaagttgaaataaggctagtccgttatcaacttgaaaaagtggcaccgagtcggtgcctgaccaccctgtgagtccatggcccgtaggatgagaaaaaaaaagctgccatcagtcggcgtggactgtagaacactgccaatgccggtcccaagcccggataaaagtggagggtacagtccacgctttttttgcggaactggatgctgaccaccctgtgattccatggcccgtaggatgagattttttcagtgcactcgcgctaagcagcttggcgtaactagatct

SEQ ID NO: 256 - DNA, 350 bp, unidentified
  source 1..350: mol_type = unassigned DNA
ggccaaagcatgcatgtcatacctgtaattcacaagtttcagagctatgctggaaacagcatagcaagttgaaataaggctagtccgttatcaacttgaaaaagtggcaccgagtcggtgcttggtgaattacaggtaaaaaaaaaagctgccatcagtcggcgtggactgtagaacactgccaatgccggtcccaagcccggataaaagtggagggtacagtccacgcttttttttgcaaacaaggagactccataaaggcatatggtgctgggctcctgtcatcctttgtgaattacaggtatgaccttcacaggccgcctgctaagcagcttggcgtaactagatct

SEQ ID NO: 257 - DNA, 350 bp, unidentified
  source 1..350: mol_type = unassigned DNA
ggccaaagcatgcatgtcatacctgtaattcacaagtttcagagctatgctggaaacagcatagcaagttgaaataaggctagtccgttatcaacttgaaaaagtggcaccgagtcggtgctcctttggtgaattacaggtaaaaaaaaaagctgccatcagtcggcgtggactgtagaacactgccaatgccggtcccaagcccggataaaagtggagggtacagtccacgctttttttaacaaggagactccataaaggcatatggtgctgggctcctgtcatcctttgtgaattacaggtatgaccttcacagggattaagctaagcagcttggcgtaactagatct

SEQ ID NO: 258 - DNA, 350 bp, unidentified
  source 1..350: mol_type = unassigned DNA
ggccaaagcatgcatgtcatacctgtaattcacaagtttcagagctatgctggaaacagcatagcaagttgaaataaggctagtccgttatcaacttgaaaaagtggcaccgagtcggtgctcctgtcatcctttggtgaattacaggtaaaaaaaaaagctgccatcagtcggcgtggactgtagaacactgccaatgccggtcccaagcccggataaaagtggagggtacagtccacgctttttttgactccataaaggcatatggtgctgggctcctgtcatcctttgtgaattacaggtatgaccttcacagggagctggctaagcagcttggcgtaactagatct

SEQ ID NO: 259 - DNA, 350 bp, unidentified
  source 1..350: mol_type = unassigned DNA
ggccaaagcatgcatgggctgtcttctccagctccgtttcagagctatgctggaaacagcatagcaagttgaaataaggctagtccgttatcaacttgaaaaagtggcaccgagtcggtgcaaagcttctccccctggagctggagaagacaaaaaaaaaagctgccatcagtcggcgtggactgtagaacactgccaatgccggtcccaagcccggataaaagtggagggtacagtccacgctttttttacagtactgcttatcagagaagccaaacttctccccctggagctggagaagacagccatccaaaattgcatgtgctaagcagcttggcgtaactagatct

SEQ ID NO: 260 - DNA, 350 bp, unidentified
  source 1..350: mol_type = unassigned DNA
ggccaaagcatgcatgggctgtcttctccagctccgtttcagagctatgctggaaacagcatagcaagttgaaataaggctagtccgttatcaacttgaaaaagtggcaccgagtcggtgcagaagccaaagcttctccccctggagctggagaagacaaaaaaaaaagctgccatcagtcggcgtggactgtagaacactgccaatgccggtcccaagcccggataaaagtggagggtacagtccacgcttttttttgcttatcagagaagccaaacttctccccctggagctggagaagacagccatccaaaattaagtctgctaagcagcttggcgtaactagatct

SEQ ID NO: 261 - DNA, 350 bp, unidentified
  source 1..350: mol_type = unassigned DNA
ggccaaagcatgcatgggctgtcttctccagctccgtttcagagctatgctggaaacagcatagcaagttgaaataaggctagtccgttatcaacttgaaaaagtggcaccgagtcggtgcatcagagaagccaaagcttctccccctggagctggagaagacaaaaaaaaaagctgccatcagtcggcgtggactgtagaacactgccaatgccggtcccaagcccggataaaagtggagggtacagtccacgctttttttatcagagaagccaaacttctccccctggagctggagaagacagccatccaaaattcgacgcgctaagcagcttggcgtaactagatct

SEQ ID NO: 262 - DNA, 350 bp, unidentified
  source 1..350: mol_type = unassigned DNA
ggccaaagcatgcatgctgtcttctccagctccaggtttcagagctatgctggaaacagcatagcaagttgaaataaggctagtccgttatcaacttgaaaaagtggcaccgagtcggtgcaaagcttctccccctggagctggagaagaaaaaaaaaagctgccatcagtcggcgtggactgtagaacactgccaatgccggtcccaagcccggataaaagtggagggtacagtccacgctttttttgcctacagtactgcttatcagagaagccaaacttctccccctggagctggagaagacagccatccaaaagtgccggctaagcagcttggcgtaactagatct

SEQ ID NO: 263 - DNA, 350 bp, unidentified
  source 1..350: mol_type = unassigned DNA
ggccaaagcatgcatgctgtcttctccagctccaggtttcagagctatgctggaaacagcatagcaagttgaaataaggctagtccgttatcaacttgaaaaagtggcaccgagtcggtgcagaagccaaagcttctccccctggagctggagaagaaaaaaaaaagctgccatcagtcggcgtggactgtagaacactgccaatgccggtcccaagcccggataaaagtggagggtacagtccacgctttttttgtactgcttatcagagaagccaaacttctccccctggagctggagaagacagccatccaaaagcgggtgctaagcagcttggcgtaactagatct

SEQ ID NO: 264 - DNA, 350 bp, unidentified
  source 1..350: mol_type = unassigned DNA
ggccaaagcatgcatgctgtcttctccagctccaggtttcagagctatgctggaaacagcatagcaagttgaaataaggctagtccgttatcaacttgaaaaagtggcaccgagtcggtgcatcagagaagccaaagcttctccccctggagctggagaagaaaaaaaaaagctgccatcagtcggcgtggactgtagaacactgccaatgccggtcccaagcccggataaaagtggagggtacagtccacgctttttttgcttatcagagaagccaaacttctccccctggagctggagaagacagccatccaaaatctcttgctaagcagcttggcgtaactagatct

SEQ ID NO: 265 - DNA, 350 bp, unidentified
  source 1..350: mol_type = unassigned DNA
ggccaaagcatgcatgagctccagggggagaagttgtttcagagctatgctggaaacagcatagcaagttgaaataaggctagtccgttatcaacttgaaaaagtggcaccgagtcggtgcaaagcttctccccctggaaaaaaaaaagctgccatcagtcggcgtggactgtagaacactgccaatgccggtcccaagcccggataaaagtggagggtacagtccacgcttttttttgataataacttttcacttggggcctacagtactgcttatcagagaagccaaacttctccccctggagctggagaagacaaattcggctaagcagcttggcgtaactagatct

SEQ ID NO: 266 - DNA, 350 bp, unidentified
  source 1..350: mol_type = unassigned DNA
ggccaaagcatgcatgagctccagggggagaagttgtttcagagctatgctggaaacagcatagcaagttgaaataaggctagtccgttatcaacttgaaaaagtggcaccgagtcggtgcagccaaagcttctccccctggaaaaaaaaaagctgccatcagtcggcgtggactgtagaacactgccaatgccggtcccaagcccggataaaagtggagggtacagtccacgctttttttaataacttttcacttggggcctacagtactgcttatcagagaagccaaacttctccccctggagctggagaagacaagtaacgctaagcagcttggcgtaactagatct

SEQ ID NO: 267 - DNA, 350 bp, unidentified
  source 1..350: mol_type = unassigned DNA
ggccaaagcatgcatgagctccagggggagaagttgtttcagagctatgctggaaacagcatagcaagttgaaataaggctagtccgttatcaacttgaaaaagtggcaccgagtcggtgcagaagccaaagcttctccccctggaaaaaaaaaagctgccatcagtcggcgtggactgtagaacactgccaatgccggtcccaagcccggataaaagtggagggtacagtccacgctttttttaacttttcacttggggcctacagtactgcttatcagagaagccaaacttctccccctggagctggagaagacaacatgcgctaagcagcttggcgtaactagatct

SEQ ID NO: 268 - DNA, 350 bp, unidentified
  source 1..350: mol_type = unassigned DNA
ggccaaagcatgcatgagctccagggggagaagttgtttcagagctatgctggaaacagcatagcaagttgaaataaggctagtccgttatcaacttgaaaaagtggcaccgagtcggtgcatcagagaagccaaagcttctccccctggaaaaaaaaaagctgccatcagtcggcgtggactgtagaacactgccaatgccggtcccaagcccggataaaagtggagggtacagtccacgctttttttttcacttggggcctacagtactgcttatcagagaagccaaacttctccccctggagctggagaagacagtccaggctaagcagcttggcgtaactagatct

SEQ ID NO: 269 - DNA, 350 bp, unidentified
  source 1..350: mol_type = unassigned DNA
ggccaaagcatgcatgaaaataaagtgcagctgaagtttcagagctatgctggaaacagcatagcaagttgaaataaggctagtccgttatcaacttgaaaaagtggcaccgagtcggtgctcacggcccttcagctgcactttataaaaaaaaagctgccatcagtcggcgtggactgtagaacactgccaatgccggtcccaagcccggataaaagtggagggtacagtccacgcttttttttttcttctccggtaaagttttttagagtgagaaggtcatggcccttcagctgcactttattttgtagtggttgagaatggctaagcagcttggcgtaactagatct

SEQ ID NO: 270 - DNA, 350 bp, unidentified
  source 1..350: mol_type = unassigned DNA
ggccaaagcatgcatgaaaataaagtgcagctgaagtttcagagctatgctggaaacagcatagcaagttgaaataaggctagtccgttatcaacttgaaaaagtggcaccgagtcggtgcaaggtcacggcccttcagctgcactttataaaaaaaaagctgccatcagtcggcgtggactgtagaacactgccaatgccggtcccaagcccggataaaagtggagggtacagtccacgctttttttttctccggtaaagttttttagagtgagaaggtcatggcccttcagctgcactttattttgtagtggttgcttatagctaagcagcttggcgtaactagatct

SEQ ID NO: 271 - DNA, 350 bp, unidentified
  source 1..350: mol_type = unassigned DNA
ggccaaagcatgcatgaaaataaagtgcagctgaagtttcagagctatgctggaaacagcatagcaagttgaaataaggctagtccgttatcaacttgaaaaagtggcaccgagtcggtgcgagaaggtcacggcccttcagctgcactttataaaaaaaaagctgccatcagtcggcgtggactgtagaacactgccaatgccggtcccaagcccggataaaagtggagggtacagtccacgcttttttttccggtaaagttttttagagtgagaaggtcatggcccttcagctgcactttattttgtagtggttggttaatgctaagcagcttggcgtaactagatct

SEQ ID NO: 272 - DNA, 350 bp, unidentified
  source 1..350: mol_type = unassigned DNA
ggccaaagcatgcatgaaaataaagtgcagctgaagtttcagagctatgctggaaacagcatagcaagttgaaataaggctagtccgttatcaacttgaaaaagtggcaccgagtcggtgcagagtgagaaggtcacggcccttcagctgcactttataaaaaaaaagctgccatcagtcggcgtggactgtagaacactgccaatgccggtcccaagcccggataaaagtggagggtacagtccacgcttttttttaaagttttttagagtgagaaggtcatggcccttcagctgcactttattttgtagtggttgatactggctaagcagcttggcgtaactagatct

SEQ ID NO: 273 - DNA, 350 bp, unidentified
  source 1..350: mol_type = unassigned DNA
ggccaaagcatgcatgaaagttttttagagtgagagtttcagagctatgctggaaacagcatagcaagttgaaataaggctagtccgttatcaacttgaaaaagtggcaccgagtcggtgcgccgtgaccttctcactctaaaaaacaaaaaaaaagctgccatcagtcggcgtggactgtagaacactgccaatgccggtcccaagcccggataaaagtggagggtacagtccacgctttttttgacaaccactacaaaataaagtgcagctgaagggccatgaccttctcactctaaaaaactttaccggagaaggaggcagctaagcagcttggcgtaactagatct

SEQ ID NO: 274 - DNA, 350 bp, unidentified
  source 1..350: mol_type = unassigned DNA
ggccaaagcatgcatgaaagttttttagagtgagagtttcagagctatgctggaaacagcatagcaagttgaaataaggctagtccgttatcaacttgaaaaagtggcaccgagtcggtgcaagggccgtgaccttctcactctaaaaaacaaaaaaaaagctgccatcagtcggcgtggactgtagaacactgccaatgccggtcccaagcccggataaaagtggagggtacagtccacgctttttttaccactacaaaataaagtgcagctgaagggccatgaccttctcactctaaaaaactttaccggagaagcttacagctaagcagcttggcgtaactagatct

SEQ ID NO: 275 - DNA, 350 bp, unidentified
  source 1..350: mol_type = unassigned DNA
ggccaaagcatgcatgaaagttttttagagtgagagtttcagagctatgctggaaacagcatagcaagttgaaataaggctagtccgttatcaacttgaaaaagtggcaccgagtcggtgcctgaagggccgtgaccttctcactctaaaaaacaaaaaaaaagctgccatcagtcggcgtggactgtagaacactgccaatgccggtcccaagcccggataaaagtggagggtacagtccacgctttttttactacaaaataaagtgcagctgaagggccatgaccttctcactctaaaaaactttaccggagaagtttaacgctaagcagcttggcgtaactagatct

SEQ ID NO: 276 - DNA, 350 bp, unidentified
  source 1..350: mol_type = unassigned DNA
ggccaaagcatgcatgaaagttttttagagtgagagtttcagagctatgctggaaacagcatagcaagttgaaataaggctagtccgttatcaacttgaaaaagtggcaccgagtcggtgctgcagctgaagggccgtgaccttctcactctaaaaaacaaaaaaaaagctgccatcagtcggcgtggactgtagaacactgccaatgccggtcccaagcccggataaaagtggagggtacagtccacgctttttttaaaataaagtgcagctgaagggccatgaccttctcactctaaaaaactttaccggagaagtgtgttgctaagcagcttggcgtaactagatct

SEQ ID NO: 277 - DNA, 350 bp, unidentified
  source 1..350: mol_type = unassigned DNA
ggccaaagcatgcatgttttagagtgagaaggtcagtttcagagctatgctggaaacagcatagcaagttgaaataaggctagtccgttatcaacttgaaaaagtggcaccgagtcggtgcgccgtgaccttctcactctaaaaaaaaaagctgccatcagtcggcgtggactgtagaacactgccaatgccggtcccaagcccggataaaagtggagggtacagtccacgcttttttttttacaggtgtggacaaccactacaaaataaagtgcagctgaagggccatgaccttctcactctaaaaaactttaccgcagaatgctaagcagcttggcgtaactagatct

SEQ ID NO: 278 - DNA, 350 bp, unidentified
  source 1..350: mol_type = unassigned DNA
ggccaaagcatgcatgttttagagtgagaaggtcagtttcagagctatgctggaaacagcatagcaagttgaaataaggctagtccgttatcaacttgaaaaagtggcaccgagtcggtgcaagggccgtgaccttctcactctaaaaaaaaaagctgccatcagtcggcgtggactgtagaacactgccaatgccggtcccaagcccggataaaagtggagggtacagtccacgctttttttcaggtgtggacaaccactacaaaataaagtgcagctgaagggccatgaccttctcactctaaaaaactttaccgacgacagctaagcagcttggcgtaactagatct

SEQ ID NO: 279 - DNA, 350 bp, unidentified
  source 1..350: mol_type = unassigned DNA
ggccaaagcatgcatgttttagagtgagaaggtcagtttcagagctatgctggaaacagcatagcaagttgaaataaggctagtccgttatcaacttgaaaaagtggcaccgagtcggtgcctgaagggccgtgaccttctcactctaaaaaaaaaagctgccatcagtcggcgtggactgtagaacactgccaatgccggtcccaagcccggataaaagtggagggtacagtccacgctttttttgtgtggacaaccactacaaaataaagtgcagctgaagggccatgaccttctcactctaaaaaactttaccgccctatgctaagcagcttggcgtaactagatct

SEQ ID NO: 280 - DNA, 350 bp, unidentified
  source 1..350: mol_type = unassigned DNA
ggccaaagcatgcatgttttagagtgagaaggtcagtttcagagctatgctggaaacagcatagcaagttgaaataaggctagtccgttatcaacttgaaaaagtggcaccgagtcggtgctgcagctgaagggccgtgaccttctcactctaaaaaaaaaagctgccatcagtcggcgtggactgtagaacactgccaatgccggtcccaagcccggataaaagtggagggtacagtccacgctttttttgacaaccactacaaaataaagtgcagctgaagggccatgaccttctcactctaaaaaactttaccgtaccacgctaagcagcttggcgtaactagatct

SEQ ID NO: 281 - DNA, 350 bp, unidentified
  source 1..350: mol_type = unassigned DNA
ggccaaagcatgcatgcctcaatcctttgggtgtagtttcagagctatgctggaaacagcatagcaagttgaaataaggctagtccgttatcaacttgaaaaagtggcaccgagtcggtgcttcgctacgacccatacacccaaaggattgaaaaaaaaagctgccatcagtcggcgtggactgtagaacactgccaatgccggtcccaagcccggataaaagtggagggtacagtccacgctttttttgccacaatacctcggcccttctcagttccctacgacccatacacccaaaggattgaggtcttggacaagtgcacgctaagcagcttggcgtaactagatct

SEQ ID NO: 282 - DNA, 350 bp, unidentified
  source 1..350: mol_type = unassigned DNA
ggccaaagcatgcatgcctcaatcctttgggtgtagtttcagagctatgctggaaacagcatagcaagttgaaataaggctagtccgttatcaacttgaaaaagtggcaccgagtcggtgcggcccttctcagttcgctacgacccatacacccaaaggattgaaaaaaaaagctgccatcagtcggcgtggactgtagaacactgccaatgccggtcccaagcccggataaaagtggagggtacagtccacgctttttttcggcccttctcagttccctacgacccatacacccaaaggattgaggtcttggacaagttagagctaagcagcttggcgtaactagatct

SEQ ID NO: 283 - DNA, 350 bp, unidentified
  source 1..350: mol_type = unassigned DNA
ggccaaagcatgcatgctttgggtgtatgggtcgtgtttcagagctatgctggaaacagcatagcaagttgaaataaggctagtccgttatcaacttgaaaaagtggcaccgagtcggtgcttcgctacgacccatacacccaaaaaaaaaagctgccatcagtcggcgtggactgtagaacactgccaatgccggtcccaagcccggataaaagtggagggtacagtccacgcttttttttcttaggaactttgctgccacaatacctcggcccttctcagttccctacgacccatacacccaaaggattgaggtcgtgatcgctaagcagcttggcgtaactagatct

SEQ ID NO: 284 - DNA, 350 bp, unidentified
  source 1..350: mol_type = unassigned DNA
ggccaaagcatgcatgctttgggtgtatgggtcgtgtttcagagctatgctggaaacagcatagcaagttgaaataaggctagtccgttatcaacttgaaaaagtggcaccgagtcggtgctcagttcgctacgacccatacacccaaaaaaaaaagctgccatcagtcggcgtggactgtagaacactgccaatgccggtcccaagcccggataaaagtggagggtacagtccacgctttttttaggaactttgctgccacaatacctcggcccttctcagttccctacgacccatacacccaaaggattgaggtccgataggctaagcagcttggcgtaactagatct

SEQ ID NO: 285 - DNA, 350 bp, unidentified
  source 1..350: mol_type = unassigned DNA
ggccaaagcatgcatgctttgggtgtatgggtcgtgtttcagagctatgctggaaacagcatagcaagttgaaataaggctagtccgttatcaacttgaaaaagtggcaccgagtcggtgcttctcagttcgctacgacccatacacccaaaaaaaaaagctgccatcagtcggcgtggactgtagaacactgccaatgccggtcccaagcccggataaaagtggagggtacagtccacgctttttttaactttgctgccacaatacctcggcccttctcagttccctacgacccatacacccaaaggattgaggtcacggcggctaagcagcttggcgtaactagatct

SEQ ID NO: 286 - DNA, 350 bp, unidentified
  source 1..350: mol_type = unassigned DNA
ggccaaagcatgcatgctttgggtgtatgggtcgtgtttcagagctatgctggaaacagcatagcaagttgaaataaggctagtccgttatcaacttgaaaaagtggcaccgagtcggtgcggcccttctcagttcgctacgacccatacacccaaaaaaaaaagctgccatcagtcggcgtggactgtagaacactgccaatgccggtcccaagcccggataaaagtggagggtacagtccacgcttttttttgctgccacaatacctcggcccttctcagttccctacgacccatacacccaaaggattgaggtctccgaagctaagcagcttggcgtaactagatct

SEQ ID NO: 287 - DNA, 350 bp, unidentified
  source 1..350: mol_type = unassigned DNA
ggccaaagcatgcatgtttgggtgtatgggtcgtagtttcagagctatgctggaaacagcatagcaagttgaaataaggctagtccgttatcaacttgaaaaagtggcaccgagtcggtgcttcgctacgacccatacacccaaaaaaaaagctgccatcagtcggcgtggactgtagaacactgccaatgccggtcccaagcccggataaaagtggagggtacagtccacgctttttttggtcttaggaactttgctgccacaatacctcggcccttctcagttccctacgacccatacacccaaaggattgaggtcggggcgctaagcagcttggcgtaactagatct

SEQ ID NO: 288 - DNA, 350 bp, unidentified
  source 1..350: mol_type = unassigned DNA
ggccaaagcatgcatgtttgggtgtatgggtcgtagtttcagagctatgctggaaacagcatagcaagttgaaataaggctagtccgttatcaacttgaaaaagtggcaccgagtcggtgctcagttcgctacgacccatacacccaaaaaaaaagctgccatcagtcggcgtggactgtagaacactgccaatgccggtcccaagcccggataaaagtggagggtacagtccacgctttttttttaggaactttgctgccacaatacctcggcccttctcagttccctacgacccatacacccaaaggattgaggtatatgagctaagcagcttggcgtaactagatct

SEQ ID NO: 289 - DNA, 350 bp, unidentified
  source 1..350: mol_type = unassigned DNA
ggccaaagcatgcatgtttgggtgtatgggtcgtagtttcagagctatgctggaaacagcatagcaagttgaaataaggctagtccgttatcaacttgaaaaagtggcaccgagtcggtgcttctcagttcgctacgacccatacacccaaaaaaaaagctgccatcagtcggcgtggactgtagaacactgccaatgccggtcccaagcccggataaaagtggagggtacagtccacgctttttttggaactttgctgccacaatacctcggcccttctcagttccctacgacccatacacccaaaggattgaggttaaagcgctaagcagcttggcgtaactagatct

SEQ ID NO: 290 - DNA, 350 bp, unidentified
  source 1..350: mol_type = unassigned DNA
ggccaaagcatgcatgtttgggtgtatgggtcgtagtttcagagctatgctggaaacagcatagcaagttgaaataaggctagtccgttatcaacttgaaaaagtggcaccgagtcggtgcggcccttctcagttcgctacgacccatacacccaaaaaaaaagctgccatcagtcggcgtggactgtagaacactgccaatgccggtcccaagcccggataaaagtggagggtacagtccacgcttttttttttgctgccacaatacctcggcccttctcagttccctacgacccatacacccaaaggattgaggtttgcctgctaagcagcttggcgtaactagatct

SEQ ID NO: 291 - DNA, 350 bp, unidentified
  source 1..350: mol_type = unassigned DNA
ggccaaagcatgcatgatccgttcaatgtctgctagtttcagagctatgctggaaacagcatagcaagttgaaataaggctagtccgttatcaacttgaaaaagtggcaccgagtcggtgcacgtatgaccttagcagacattgaacgaaaaaaaaagctgccatcagtcggcgtggactgtagaacactgccaatgccggtcccaagcccggataaaagtggagggtacagtccacgcttttttttatatttgttgcttgtgtttgtttttcccctacagacgaccttagcagacattgaacggattgctcctctggtgattgctaagcagcttggcgtaactagatct

SEQ ID NO: 292 - DNA, 350 bp, unidentified
  source 1..350: mol_type = unassigned DNA
ggccaaagcatgcatgatccgttcaatgtctgctagtttcagagctatgctggaaacagcatagcaagttgaaataaggctagtccgttatcaacttgaaaaagtggcaccgagtcggtgcacagacgtatgaccttagcagacattgaacgaaaaaaaaagctgccatcagtcggcgtggactgtagaacactgccaatgccggtcccaagcccggataaaagtggagggtacagtccacgcttttttttttgttgcttgtgtttgtttttcccctacagacgaccttagcagacattgaacggattgctcctctggtcgacgctaagcagcttggcgtaactagatct

SEQ ID NO: 293 - DNA, 350 bp, unidentified
  source 1..350: mol_type = unassigned DNA
ggccaaagcatgcatgatccgttcaatgtctgctagtttcagagctatgctggaaacagcatagcaagttgaaataaggctagtccgttatcaacttgaaaaagtggcaccgagtcggtgccctacagacgtatgaccttagcagacattgaacgaaaaaaaaagctgccatcagtcggcgtggactgtagaacactgccaatgccggtcccaagcccggataaaagtggagggtacagtccacgctttttttgttgcttgtgtttgtttttcccctacagacgaccttagcagacattgaacggattgctcctctgtcgactgctaagcagcttggcgtaactagatct

SEQ ID NO: 294 - DNA, 350 bp, unidentified
  source 1..350: mol_type = unassigned DNA
ggccaaagcatgcatgatccgttcaatgtctgctagtttcagagctatgctggaaacagcatagcaagttgaaataaggctagtccgttatcaacttgaaaaagtggcaccgagtcggtgctttcccctacagacgtatgaccttagcagacattgaacgaaaaaaaaagctgccatcagtcggcgtggactgtagaacactgccaatgccggtcccaagcccggataaaagtggagggtacagtccacgctttttttttgtgtttgtttttcccctacagacgaccttagcagacattgaacggattgctcctctgaattgtgctaagcagcttggcgtaactagatct

SEQ ID NO: 295 - DNA, 350 bp, unidentified
  source 1..350: mol_type = unassigned DNA
ggccaaagcatgcatggggtcgtagcgaactgagagtttcagagctatgctggaaacagcatagcaagttgaaataaggctagtccgttatcaacttgaaaaagtggcaccgagtcggtgcctcggcccttctcagttcgctacgaaaaaaaaaagctgccatcagtcggcgtggactgtagaacactgccaatgccggtcccaagcccggataaaagtggagggtacagtccacgcttttttttggttttggtcttaggaactttgctgccacaatacctcagcccttctcagttcgctacgacccatacacccaagaaagagctaagcagcttggcgtaactagatct

SEQ ID NO: 296 - DNA, 350 bp, unidentified
  source 1..350: mol_type = unassigned DNA
ggccaaagcatgcatggggtcgtagcgaactgagagtttcagagctatgctggaaacagcatagcaagttgaaataaggctagtccgttatcaacttgaaaaagtggcaccgagtcggtgcatacctcggcccttctcagttcgctacgaaaaaaaaaagctgccatcagtcggcgtggactgtagaacactgccaatgccggtcccaagcccggataaaagtggagggtacagtccacgcttttttttttggtcttaggaactttgctgccacaatacctcagcccttctcagttcgctacgacccatacacccaaaatactgctaagcagcttggcgtaactagatct

SEQ ID NO: 297 - DNA, 350 bp, unidentified
  source 1..350: mol_type = unassigned DNA
ggccaaagcatgcatggggtcgtagcgaactgagagtttcagagctatgctggaaacagcatagcaagttgaaataaggctagtccgttatcaacttgaaaaagtggcaccgagtcggtgcacaatacctcggcccttctcagttcgctacgaaaaaaaaaagctgccatcagtcggcgtggactgtagaacactgccaatgccggtcccaagcccggataaaagtggagggtacagtccacgctttttttggtcttaggaactttgctgccacaatacctcagcccttctcagttcgctacgacccatacacccaaccatccgctaagcagcttggcgtaactagatct

SEQ ID NO: 298 - DNA, 350 bp, unidentified
  source 1..350: mol_type = unassigned DNA
ggccaaagcatgcatggggtcgtagcgaactgagagtttcagagctatgctggaaacagcatagcaagttgaaataaggctagtccgttatcaacttgaaaaagtggcaccgagtcggtgcctgccacaatacctcggcccttctcagttcgctacgaaaaaaaaaagctgccatcagtcggcgtggactgtagaacactgccaatgccggtcccaagcccggataaaagtggagggtacagtccacgcttttttttaggaactttgctgccacaatacctcagcccttctcagttcgctacgacccatacacccaaacgtcagctaagcagcttggcgtaactagatct

SEQ ID NO: 299 - DNA, 350 bp, unidentified
  source 1..350: mol_type = unassigned DNA
ggccaaagcatgcatgggtcgtagcgaactgagaagtttcagagctatgctggaaacagcatagcaagttgaaataaggctagtccgttatcaacttgaaaaagtggcaccgagtcggtgcatacctcggcccttctcagttcgctacgaaaaaaaaagctgccatcagtcggcgtggactgtagaacactgccaatgccggtcccaagcccggataaaagtggagggtacagtccacgctttttttgttttggtcttaggaactttgctgccacaatacctcagcccttctcagttcgctacgacccatacacccatgagaagctaagcagcttggcgtaactagatct

SEQ ID NO: 300 - DNA, 350 bp, unidentified
  source 1..350: mol_type = unassigned DNA
ggccaaagcatgcatgggtcgtagcgaactgagaagtttcagagctatgctggaaacagcatagcaagttgaaataaggctagtccgttatcaacttgaaaaagtggcaccgagtcggtgcacaatacctcggcccttctcagttcgctacgaaaaaaaaagctgccatcagtcggcgtggactgtagaacactgccaatgccggtcccaagcccggataaaagtggagggtacagtccacgctttttttttggtcttaggaactttgctgccacaatacctcagcccttctcagttcgctacgacccatacacccaagtagcgctaagcagcttggcgtaactagatct

SEQ ID NO: 301 - DNA, 350 bp, unidentified
  source 1..350: mol_type = unassigned DNA
ggccaaagcatgcatgagcgaactgagaagggctggtttcagagctatgctggaaacagcatagcaagttgaaataaggctagtccgttatcaacttgaaaaagtggcaccgagtcggtgcatacctcggcccttctcagttcaaaaaaaaagctgccatcagtcggcgtggactgtagaacactgccaatgccggtcccaagcccggataaaagtggagggtacagtccacgctttttttctcaagcctgtggttttggtcttaggaactttgctgccacaatacctcagcccttctcagttcgctacgacccataccgactgctaagcagcttggcgtaactagatct

SEQ ID NO: 302 - DNA, 350 bp, unidentified
  source 1..350: mol_type = unassigned DNA
ggccaaagcatgcatgagcgaactgagaagggctggtttcagagctatgctggaaacagcatagcaagttgaaataaggctagtccgttatcaacttgaaaaagtggcaccgagtcggtgcacaatacctcggcccttctcagttcaaaaaaaaagctgccatcagtcggcgtggactgtagaacactgccaatgccggtcccaagcccggataaaagtggagggtacagtccacgctttttttaagcctgtggttttggtcttaggaactttgctgccacaatacctcagcccttctcagttcgctacgacccatattgcatgctaagcagcttggcgtaactagatct

SEQ ID NO: 303 - DNA, 350 bp, unidentified
  source 1..350: mol_type = unassigned DNA
ggccaaagcatgcatgtcccccaattacaggaagtgtttcagagctatgctggaaacagcatagcaagttgaaataaggctagtccgttatcaacttgaaaaagtggcaccgagtcggtgccaatttcctgtaattgggaaaaaaaaagctgccatcagtcggcgtggactgtagaacactgccaatgccggtcccaagcccggataaaagtggagggtacagtccacgctttttttgtggcgagcttttcaatgtattcatcaggtgcacccagagaggcaaggccaacttcctgtaattgggggaaaatagaaccggtgaagctaagcagcttggcgtaactagatct

SEQ ID NO: 304 - DNA, 350 bp, unidentified
  source 1..350: mol_type = unassigned DNA
ggccaaagcatgcatgtcccccaattacaggaagtgtttcagagctatgctggaaacagcatagcaagttgaaataaggctagtccgttatcaacttgaaaaagtggcaccgagtcggtgcaggccaatttcctgtaattgggaaaaaaaaagctgccatcagtcggcgtggactgtagaacactgccaatgccggtcccaagcccggataaaagtggagggtacagtccacgctttttttcgagcttttcaatgtattcatcaggtgcacccagagaggcaaggccaacttcctgtaattgggggaaaatagaaccaatggagctaagcagcttggcgtaactagatct

SEQ ID NO: 305 - DNA, 350 bp, unidentified
  source 1..350: mol_type = unassigned DNA
ggccaaagcatgcatgtcccccaattacaggaagtgtttcagagctatgctggaaacagcatagcaagttgaaataaggctagtccgttatcaacttgaaaaagtggcaccgagtcggtgcgcaaggccaatttcctgtaattgggaaaaaaaaagctgccatcagtcggcgtggactgtagaacactgccaatgccggtcccaagcccggataaaagtggagggtacagtccacgctttttttgcttttcaatgtattcatcaggtgcacccagagaggcaaggccaacttcctgtaattgggggaaaatagaaccagcgtagctaagcagcttggcgtaactagatct

SEQ ID NO: 306 - DNA, 350 bp, unidentified
  source 1..350: mol_type = unassigned DNA
ggccaaagcatgcatgtcccccaattacaggaagtgtttcagagctatgctggaaacagcatagcaagttgaaataaggctagtccgttatcaacttgaaaaagtggcaccgagtcggtgcgagaggcaaggccaatttcctgtaattgggaaaaaaaaagctgccatcagtcggcgtggactgtagaacactgccaatgccggtcccaagcccggataaaagtggagggtacagtccacgcttttttttcaatgtattcatcaggtgcacccagagaggcaaggccaacttcctgtaattgggggaaaatagaaccatctccgctaagcagcttggcgtaactagatct

SEQ ID NO: 307 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgcttccgagtcttccactgcacacagtacatcaaaaaaaaagctgccatcagtcggcgtggactgtagaacactgccaatgccggtcccaagcccggataaaagtggagggtacagtccacgcttttttttcgggatttcttgggtggcctggccttctgagtcttccactgcacacagtacatcagacatggatccagcggaggctaagcagcttggcgtaactagatct

SEQ ID NO: 308 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgcggccttccgagtcttccactgcacacagtacatcaaaaaaaaagctgccatcagtcggcgtggactgtagaacactgccaatgccggtcccaagcccggataaaagtggagggtacagtccacgctttttttgatttcttgggtggcctggccttctgagtcttccactgcacacagtacatcagacatggatccacaggctgctaagcagcttggcgtaactagatct

SEQ ID NO: 309 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgccctggccttccgagtcttccactgcacacagtacatcaaaaaaaaagctgccatcagtcggcgtggactgtagaacactgccaatgccggtcccaagcccggataaaagtggagggtacagtccacgctttttttttcttgggtggcctggccttctgagtcttccactgcacacagtacatcagacatggatccaatgcccgctaagcagcttggcgtaactagatct

SEQ ID NO: 310 - DNA, 350 bp, unidentified
  source 1..350: mol_type = unassigned DNA
ggccaaagcatgcatgtctgatgtactgtgtgcaggtttcagagctatgctggaaacagcatagcaagttgaaataaggctagtccgttatcaacttgaaaaagtggcaccgagtcggtgcggtggcctggccttccgagtcttccactgcacacagtacatcaaaaaaaaagctgccatcagtcggcgtggactgtagaacactgccaatgccggtcccaagcccggataaaagtggagggtacagtccacgctttttttgggtggcctggccttctgagtcttccactgcacacagtacatcagacatggatccaagatttgctaagcagcttggcgtaactagatct

SEQ ID NO: 311 - DNA, 350 bp, unidentified
  source 1..350: mol_type = unassigned DNA
ggccaaagcatgcatggtgcagtggaagactcagagtttcagagctatgctggaaacagcatagcaagttgaaataaggctagtccgttatcaacttgaaaaagtggcaccgagtcggtgcttccgagtcttccactgaaaaaaaaagctgccatcagtcggcgtggactgtagaacactgccaatgccggtcccaagcccggataaaagtggagggtacagtccacgctttttttcgacctgtggctggcctgctttcctctcgggatttcttgggtggcctggccttctgagtcttccactgcacacagtacatccgtggtgctaagcagcttggcgtaactagatct

SEQ ID NO: 312 - DNA, 350 bp, unidentified
  source 1..350: mol_type = unassigned DNA
ggccaaagcatgcatggtgcagtggaagactcagagtttcagagctatgctggaaacagcatagcaagttgaaataaggctagtccgttatcaacttgaaaaagtggcaccgagtcggtgcggccttccgagtcttccactgaaaaaaaaagctgccatcagtcggcgtggactgtagaacactgccaatgccggtcccaagcccggataaaagtggagggtacagtccacgctttttttctgtggctggcctgctttcctctcgggatttcttgggtggcctggccttctgagtcttccactgcacacagtacatcatgttggctaagcagcttggcgtaactagatct

SEQ ID NO: 313 - DNA, 350 bp, unidentified
  source 1..350: mol_type = unassigned DNA
ggccaaagcatgcatggtgcagtggaagactcagagtttcagagctatgctggaaacagcatagcaagttgaaataaggctagtccgttatcaacttgaaaaagtggcaccgagtcggtgccctggccttccgagtcttccactgaaaaaaaaagctgccatcagtcggcgtggactgtagaacactgccaatgccggtcccaagcccggataaaagtggagggtacagtccacgcttttttttggctggcctgctttcctctcgggatttcttgggtggcctggccttctgagtcttccactgcacacagtacatcttagaggctaagcagcttggcgtaactagatct

SEQ ID NO: 314 - DNA, 350 bp, unidentified
  source 1..350: mol_type = unassigned DNA
ggccaaagcatgcatggtgcagtggaagactcagagtttcagagctatgctggaaacagcatagcaagttgaaataaggctagtccgttatcaacttgaaaaagtggcaccgagtcggtgcggtggcctggccttccgagtcttccactgaaaaaaaaagctgccatcagtcggcgtggactgtagaacactgccaatgccggtcccaagcccggataaaagtggagggtacagtccacgctttttttggcctgctttcctctcgggatttcttgggtggcctggccttctgagtcttccactgcacacagtacatcttcagtgctaagcagcttggcgtaactagatct

SEQ ID NO: 315 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgcctcggaaggccaggccacccaagaaatcaaaaaaaaagctgccatcagtcggcgtggactgtagaacactgccaatgccggtcccaagcccggataaaagtggagggtacagtccacgctttttttatgtctgatgtactgtgtgcagtggaagactcagaaggccaggccacccaagaaatcccgagaggaaagcaaagtcgctaagcagcttggcgtaactagatct

SEQ ID NO: 316 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgcaagactcggaaggccaggccacccaagaaatcaaaaaaaaagctgccatcagtcggcgtggactgtagaacactgccaatgccggtcccaagcccggataaaagtggagggtacagtccacgctttttttctgatgtactgtgtgcagtggaagactcagaaggccaggccacccaagaaatcccgagaggaaagcctggtcgctaagcagcttggcgtaactagatct

SEQ ID NO: 317 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgctggaagactcggaaggccaggccacccaagaaatcaaaaaaaaagctgccatcagtcggcgtggactgtagaacactgccaatgccggtcccaagcccggataaaagtggagggtacagtccacgctttttttatgtactgtgtgcagtggaagactcagaaggccaggccacccaagaaatcccgagaggaaagcgtccctgctaagcagcttggcgtaactagatct

SEQ ID NO: 318 - DNA, 350 bp, unidentified
  source 1..350: mol_type = unassigned DNA
ggccaaagcatgcatgcgggatttcttgggtggccgtttcagagctatgctggaaacagcatagcaagttgaaataaggctagtccgttatcaacttgaaaaagtggcaccgagtcggtgctgcagtggaagactcggaaggccaggccacccaagaaatcaaaaaaaaagctgccatcagtcggcgtggactgtagaacactgccaatgccggtcccaagcccggataaaagtggagggtacagtccacgctttttttctgtgtgcagtggaagactcagaaggccaggccacccaagaaatcccgagaggaaagctagttcgctaagcagcttggcgtaactagatct

SEQ ID NO: 319 - DNA, 350 bp, unidentified
  source 1..350: mol_type = unassigned DNA
ggccaaagcatgcatgtgagcagctcaggctgccggtttcagagctatgctggaaacagcatagcaagttgaaataaggctagtccgttatcaacttgaaaaagtggcaccgagtcggtgctgagccacggcagcctgagctgcaaaaaaaaagctgccatcagtcggcgtggactgtagaacactgccaatgccggtcccaagcccggataaaagtggagggtacagtccacgctttttttacccaggcttgggcaggaaactctctgactttggacaggtgatccacggcagcctgagctgctcagttaggggaagaggtggctaagcagcttggcgtaactagatct

SEQ ID NO: 320 - DNA, 350 bp, unidentified
  source 1..350: mol_type = unassigned DNA
ggccaaagcatgcatgtgagcagctcaggctgccggtttcagagctatgctggaaacagcatagcaagttgaaataaggctagtccgttatcaacttgaaaaagtggcaccgagtcggtgccaggtgagccacggcagcctgagctgcaaaaaaaaagctgccatcagtcggcgtggactgtagaacactgccaatgccggtcccaagcccggataaaagtggagggtacagtccacgctttttttaggcttgggcaggaaactctctgactttggacaggtgatccacggcagcctgagctgctcagttaggggaaatagccgctaagcagcttggcgtaactagatct

SEQ ID NO: 321 - DNA, 350 bp, unidentified
  source 1..350: mol_type = unassigned DNA
ggccaaagcatgcatgtgagcagctcaggctgccggtttcagagctatgctggaaacagcatagcaagttgaaataaggctagtccgttatcaacttgaaaaagtggcaccgagtcggtgcggacaggtgagccacggcagcctgagctgcaaaaaaaaagctgccatcagtcggcgtggactgtagaacactgccaatgccggtcccaagcccggataaaagtggagggtacagtccacgctttttttcttgggcaggaaactctctgactttggacaggtgatccacggcagcctgagctgctcagttaggggaatcactggctaagcagcttggcgtaactagatct

SEQ ID NO: 322 - DNA, 350 bp, unidentified
  source 1..350: mol_type = unassigned DNA
ggccaaagcatgcatgtgagcagctcaggctgccggtttcagagctatgctggaaacagcatagcaagttgaaataaggctagtccgttatcaacttgaaaaagtggcaccgagtcggtgcactttggacaggtgagccacggcagcctgagctgcaaaaaaaaagctgccatcagtcggcgtggactgtagaacactgccaatgccggtcccaagcccggataaaagtggagggtacagtccacgctttttttgcaggaaactctctgactttggacaggtgatccacggcagcctgagctgctcagttaggggaatccactgctaagcagcttggcgtaactagatct

SEQ ID NO: 323 - DNA, 350 bp, unidentified
  source 1..350: mol_type = unassigned DNA
ggccaaagcatgcatggcaggaaactctctgacttgtttcagagctatgctggaaacagcatagcaagttgaaataaggctagtccgttatcaacttgaaaaagtggcaccgagtcggtgctggctcacctgtccaaagtcagagagtttccaaaaaaaaagctgccatcagtcggcgtggactgtagaacactgccaatgccggtcccaagcccggataaaagtggagggtacagtccacgcttttttttaactgagcagctcaggctgccgtggatcacctgtccaaagtcagagagtttcctgcccaagcctgggatttcgctaagcagcttggcgtaactagatct

SEQ ID NO: 324 - DNA, 350 bp, unidentified
  source 1..350: mol_type = unassigned DNA
ggccaaagcatgcatggcaggaaactctctgacttgtttcagagctatgctggaaacagcatagcaagttgaaataaggctagtccgttatcaacttgaaaaagtggcaccgagtcggtgcgccgtggctcacctgtccaaagtcagagagtttccaaaaaaaaagctgccatcagtcggcgtggactgtagaacactgccaatgccggtcccaagcccggataaaagtggagggtacagtccacgcttttttttgagcagctcaggctgccgtggatcacctgtccaaagtcagagagtttcctgcccaagcctggacgtcggctaagcagcttggcgtaactagatct

SEQ ID NO: 325 - DNA, 350 bp, unidentified
  source 1..350: mol_type = unassigned DNA
ggccaaagcatgcatggcaggaaactctctgacttgtttcagagctatgctggaaacagcatagcaagttgaaataaggctagtccgttatcaacttgaaaaagtggcaccgagtcggtgcgctgccgtggctcacctgtccaaagtcagagagtttccaaaaaaaaagctgccatcagtcggcgtggactgtagaacactgccaatgccggtcccaagcccggataaaagtggagggtacagtccacgctttttttgcagctcaggctgccgtggatcacctgtccaaagtcagagagtttcctgcccaagcctggactcgtgctaagcagcttggcgtaactagatct

SEQ ID NO: 326 - DNA, 350 bp, unidentified
  source 1..350: mol_type = unassigned DNA
ggccaaagcatgcatgaaactctctgactttggacgtttcagagctatgctggaaacagcatagcaagttgaaataaggctagtccgttatcaacttgaaaaagtggcaccgagtcggtgctggctcacctgtccaaagtcagagagaaaaaaaaagctgccatcagtcggcgtggactgtagaacactgccaatgccggtcccaagcccggataaaagtggagggtacagtccacgctttttttcaaattcccctaactgagcagctcaggctgccgtggatcacctgtccaaagtcagagagtttcctgcccaaggaggaagctaagcagcttggcgtaactagatct

SEQ ID NO: 327 - DNA, 350 bp, unidentified
  source 1..350: mol_type = unassigned DNA
ggccaaagcatgcatgaaactctctgactttggacgtttcagagctatgctggaaacagcatagcaagttgaaataaggctagtccgttatcaacttgaaaaagtggcaccgagtcggtgcgccgtggctcacctgtccaaagtcagagagaaaaaaaaagctgccatcagtcggcgtggactgtagaacactgccaatgccggtcccaagcccggataaaagtggagggtacagtccacgctttttttttcccctaactgagcagctcaggctgccgtggatcacctgtccaaagtcagagagtttcctgcccaagattcctgctaagcagcttggcgtaactagatct

SEQ ID NO: 328 - DNA, 350 bp, unidentified
  source 1..350: mol_type = unassigned DNA
ggccaaagcatgcatgaaactctctgactttggacgtttcagagctatgctggaaacagcatagcaagttgaaataaggctagtccgttatcaacttgaaaaagtggcaccgagtcggtgcgctgccgtggctcacctgtccaaagtcagagagaaaaaaaaagctgccatcagtcggcgtggactgtagaacactgccaatgccggtcccaagcccggataaaagtggagggtacagtccacgctttttttccctaactgagcagctcaggctgccgtggatcacctgtccaaagtcagagagtttcctgcccaagcgccttgctaagcagcttggcgtaactagatct

SEQ ID NO: 329 - DNA, 350 bp, unidentified
  source 1..350: mol_type = unassigned DNA
ggccaaagcatgcatgaaactctctgactttggacgtttcagagctatgctggaaacagcatagcaagttgaaataaggctagtccgttatcaacttgaaaaagtggcaccgagtcggtgcctcaggctgccgtggctcacctgtccaaagtcagagagaaaaaaaaagctgccatcagtcggcgtggactgtagaacactgccaatgccggtcccaagcccggataaaagtggagggtacagtccacgctttttttactgagcagctcaggctgccgtggatcacctgtccaaagtcagagagtttcctgcccaagtcgggtgctaagcagcttggcgtaactagatct

SEQ ID NO: 330 - DNA, 350 bp, unidentified
  source 1..350: mol_type = unassigned DNA
ggccaaagcatgcatgcttatctcgtgaaagctcagtttcagagctatgctggaaacagcatagcaagttgaaataaggctagtccgttatcaacttgaaaaagtggcaccgagtcggtgcgtgccactgtccatgagctttcacgagataaaaaaaaagctgccatcagtcggcgtggactgtagaacactgccaatgccggtcccaagcccggataaaagtggagggtacagtccacgcttttttttcatcaagatcttgaggcatgacattggtgacactgtccatgagctttcacgagataagaagaaagacaaactaagctaagcagcttggcgtaactagatct

SEQ ID NO: 331 - DNA, 350 bp, unidentified
  source 1..350: mol_type = unassigned DNA
ggccaaagcatgcatgcttatctcgtgaaagctcagtttcagagctatgctggaaacagcatagcaagttgaaataaggctagtccgttatcaacttgaaaaagtggcaccgagtcggtgcattggtgccactgtccatgagctttcacgagataaaaaaaaagctgccatcagtcggcgtggactgtagaacactgccaatgccggtcccaagcccggataaaagtggagggtacagtccacgctttttttcaagatcttgaggcatgacattggtgacactgtccatgagctttcacgagataagaagaaagacatcagttgctaagcagcttggcgtaactagatct

SEQ ID NO: 332 - DNA, 350 bp, unidentified
  source 1..350: mol_type = unassigned DNA
ggccaaagcatgcatgcttatctcgtgaaagctcagtttcagagctatgctggaaacagcatagcaagttgaaataaggctagtccgttatcaacttgaaaaagtggcaccgagtcggtgcgacattggtgccactgtccatgagctttcacgagataaaaaaaaagctgccatcagtcggcgtggactgtagaacactgccaatgccggtcccaagcccggataaaagtggagggtacagtccacgctttttttgatcttgaggcatgacattggtgacactgtccatgagctttcacgagataagaagaaagacatttcgggctaagcagcttggcgtaactagatct

SEQ ID NO: 333 - DNA, 350 bp, unidentified
  source 1..350: mol_type = unassigned DNA
ggccaaagcatgcatgcttatctcgtgaaagctcagtttcagagctatgctggaaacagcatagcaagttgaaataaggctagtccgttatcaacttgaaaaagtggcaccgagtcggtgcggcatgacattggtgccactgtccatgagctttcacgagataaaaaaaaagctgccatcagtcggcgtggactgtagaacactgccaatgccggtcccaagcccggataaaagtggagggtacagtccacgcttttttttgaggcatgacattggtgacactgtccatgagctttcacgagataagaagaaagacaacggtagctaagcagcttggcgtaactagatct

SEQ ID NO: 334 - DNA, 350 bp, unidentified
  source 1..350: mol_type = unassigned DNA
ggccaaagcatgcatgagatcttgaggcatgacatgtttcagagctatgctggaaacagcatagcaagttgaaataaggctagtccgttatcaacttgaaaaagtggcaccgagtcggtgcgtggcaccaatgtcatgcctcaagaaaaaaaaaagctgccatcagtcggcgtggactgtagaacactgccaatgccggtcccaagcccggataaaagtggagggtacagtccacgcttttttttgtctttcttcttatctcgtgaaagctcatggacagtgtcaccaatgtcatgcctcaagatcttgatgatgttgagttcgctaagcagcttggcgtaactagatct

SEQ ID NO: 335 - DNA, 350 bp, unidentified
  source 1..350: mol_type = unassigned DNA
ggccaaagcatgcatgagatcttgaggcatgacatgtttcagagctatgctggaaacagcatagcaagttgaaataaggctagtccgttatcaacttgaaaaagtggcaccgagtcggtgcgacagtggcaccaatgtcatgcctcaagaaaaaaaaaagctgccatcagtcggcgtggactgtagaacactgccaatgccggtcccaagcccggataaaagtggagggtacagtccacgcttttttttttcttcttatctcgtgaaagctcatggacagtgtcaccaatgtcatgcctcaagatcttgatgatgttgctagggctaagcagcttggcgtaactagatct

SEQ ID NO: 336 - DNA, 350 bp, unidentified
  source 1..350: mol_type = unassigned DNA
ggccaaagcatgcatgagatcttgaggcatgacatgtttcagagctatgctggaaacagcatagcaagttgaaataaggctagtccgttatcaacttgaaaaagtggcaccgagtcggtgcatggacagtggcaccaatgtcatgcctcaagaaaaaaaaaagctgccatcagtcggcgtggactgtagaacactgccaatgccggtcccaagcccggataaaagtggagggtacagtccacgctttttttcttcttatctcgtgaaagctcatggacagtgtcaccaatgtcatgcctcaagatcttgatgatgtttagttggctaagcagcttggcgtaactagatct

SEQ ID NO: 337 - DNA, 350 bp, unidentified
  source 1..350: mol_type = unassigned DNA
ggccaaagcatgcatgagatcttgaggcatgacatgtttcagagctatgctggaaacagcatagcaagttgaaataaggctagtccgttatcaacttgaaaaagtggcaccgagtcggtgcagctcatggacagtggcaccaatgtcatgcctcaagaaaaaaaaaagctgccatcagtcggcgtggactgtagaacactgccaatgccggtcccaagcccggataaaagtggagggtacagtccacgcttttttttatctcgtgaaagctcatggacagtgtcaccaatgtcatgcctcaagatcttgatgatgttcattaggctaagcagcttggcgtaactagatct

SEQ ID NO: 338 - DNA, 350 bp, unidentified
  source 1..350: mol_type = unassigned DNA
ggccaaagcatgcatggggcagcccatccctcgaggtttcagagctatgctggaaacagcatagcaagttgaaataaggctagtccgttatcaacttgaaaaagtggcaccgagtcggtgctattccactcgagggatgggctgaaaaaaaaagctgccatcagtcggcgtggactgtagaacactgccaatgccggtcccaagcccggataaaagtggagggtacagtccacgctttttttttgaacactgtgccccatgttttcttttcttcctccatgtatcccactcgagggatgggctgcccactagaatacagggtggctaagcagcttggcgtaactagatct

SEQ ID NO: 339 - DNA, 350 bp, unidentified
  source 1..350: mol_type = unassigned DNA
ggccaaagcatgcatggggcagcccatccctcgaggtttcagagctatgctggaaacagcatagcaagttgaaataaggctagtccgttatcaacttgaaaaagtggcaccgagtcggtgccatgtattccactcgagggatgggctgaaaaaaaaagctgccatcagtcggcgtggactgtagaacactgccaatgccggtcccaagcccggataaaagtggagggtacagtccacgctttttttacactgtgccccatgttttcttttcttcctccatgtatcccactcgagggatgggctgcccactagaatactatgtcgctaagcagcttggcgtaactagatct

SEQ ID NO: 340 - DNA, 350 bp, unidentified
  source 1..350: mol_type = unassigned DNA
ggccaaagcatgcatggggcagcccatccctcgaggtttcagagctatgctggaaacagcatagcaagttgaaataaggctagtccgttatcaacttgaaaaagtggcaccgagtcggtgcctccatgtattccactcgagggatgggctgaaaaaaaaagctgccatcagtcggcgtggactgtagaacactgccaatgccggtcccaagcccggataaaagtggagggtacagtccacgctttttttctgtgccccatgttttcttttcttcctccatgtatcccactcgagggatgggctgcccactagaataccttccagctaagcagcttggcgtaactagatct

SEQ ID NO: 341 - DNA, 350 bp, unidentified
  source 1..350: mol_type = unassigned DNA
ggccaaagcatgcatggggcagcccatccctcgaggtttcagagctatgctggaaacagcatagcaagttgaaataaggctagtccgttatcaacttgaaaaagtggcaccgagtcggtgctcttcctccatgtattccactcgagggatgggctgaaaaaaaaagctgccatcagtcggcgtggactgtagaacactgccaatgccggtcccaagcccggataaaagtggagggtacagtccacgctttttttccccatgttttcttttcttcctccatgtatcccactcgagggatgggctgcccactagaataccagagtgctaagcagcttggcgtaactagatct

SEQ ID NO: 342 - DNA, 350 bp, unidentified
  source 1..350: mol_type = unassigned DNA
ggccaaagcatgcatgggcagcccatccctcgagtgtttcagagctatgctggaaacagcatagcaagttgaaataaggctagtccgttatcaacttgaaaaagtggcaccgagtcggtgctcttcctccatgtattccactcgagggatgggctaaaaaaaaagctgccatcagtcggcgtggactgtagaacactgccaatgccggtcccaagcccggataaaagtggagggtacagtccacgcttttttttgccccatgttttcttttcttcctccatgtatcccactcgagggatgggctgcccactagaataacggacgctaagcagcttggcgtaactagatct

SEQ ID NO: 343 - DNA, 350 bp, unidentified
  source 1..350: mol_type = unassigned DNA
ggccaaagcatgcatgaggaagaaaagaaaacatggtttcagagctatgctggaaacagcatagcaagttgaaataaggctagtccgttatcaacttgaaaaagtggcaccgagtcggtgcgccccatgttttcttttcttaaaaaaaaagctgccatcagtcggcgtggactgtagaacactgccaatgccggtcccaagcccggataaaagtggagggtacagtccacgctttttttgcaagcatgggttttatacaaggacttcagagtcttgaacactgtgcctcatgttttcttttcttcctccatgtattcgtcaccgctaagcagcttggcgtaactagatct

SEQ ID NO: 344 - DNA, 350 bp, unidentified
  source 1..350: mol_type = unassigned DNA
ggccaaagcatgcatgaggaagaaaagaaaacatggtttcagagctatgctggaaacagcatagcaagttgaaataaggctagtccgttatcaacttgaaaaagtggcaccgagtcggtgcctgtgccccatgttttcttttcttaaaaaaaaagctgccatcagtcggcgtggactgtagaacactgccaatgccggtcccaagcccggataaaagtggagggtacagtccacgctttttttgcatgggttttatacaaggacttcagagtcttgaacactgtgcctcatgttttcttttcttcctccatgtattcctaggggctaagcagcttggcgtaactagatct

SEQ ID NO: 345 - DNA, 350 bp, unidentified
  source 1..350: mol_type = unassigned DNA
ggccaaagcatgcatgttttcatcccagcttgcacgtttcagagctatgctggaaacagcatagcaagttgaaataaggctagtccgttatcaacttgaaaaagtggcaccgagtcggtgcaggcggaaaccagtgcaagctgggatgaaaaaaaaaagctgccatcagtcggcgtggactgtagaacactgccaatgccggtcccaagcccggataaaagtggagggtacagtccacgctttttttgagaggaaagcaggccagccacaggtcggaggtggaaaccagtgcaagctgggatgaaaagaagaaagaacggatggctaagcagcttggcgtaactagatct

SEQ ID NO: 346 - DNA, 350 bp, unidentified
  source 1..350: mol_type = unassigned DNA
ggccaaagcatgcatgttttcatcccagcttgcacgtttcagagctatgctggaaacagcatagcaagttgaaataaggctagtccgttatcaacttgaaaaagtggcaccgagtcggtgctcggaggcggaaaccagtgcaagctgggatgaaaaaaaaaagctgccatcagtcggcgtggactgtagaacactgccaatgccggtcccaagcccggataaaagtggagggtacagtccacgctttttttggaaagcaggccagccacaggtcggaggtggaaaccagtgcaagctgggatgaaaagaagaaagaacgaacggctaagcagcttggcgtaactagatct

SEQ ID NO: 347 - DNA, 350 bp, unidentified
  source 1..350: mol_type = unassigned DNA
ggccaaagcatgcatgttttcatcccagcttgcacgtttcagagctatgctggaaacagcatagcaagttgaaataaggctagtccgttatcaacttgaaaaagtggcaccgagtcggtgcaggtcggaggcggaaaccagtgcaagctgggatgaaaaaaaaaagctgccatcagtcggcgtggactgtagaacactgccaatgccggtcccaagcccggataaaagtggagggtacagtccacgctttttttaagcaggccagccacaggtcggaggtggaaaccagtgcaagctgggatgaaaagaagaaagaagcagttgctaagcagcttggcgtaactagatct

SEQ ID NO: 348 - DNA, 350 bp, unidentified
  source 1..350: mol_type = unassigned DNA
ggccaaagcatgcatgttttcatcccagcttgcacgtttcagagctatgctggaaacagcatagcaagttgaaataaggctagtccgttatcaacttgaaaaagtggcaccgagtcggtgcgccacaggtcggaggcggaaaccagtgcaagctgggatgaaaaaaaaaagctgccatcagtcggcgtggactgtagaacactgccaatgccggtcccaagcccggataaaagtggagggtacagtccacgctttttttggccagccacaggtcggaggtggaaaccagtgcaagctgggatgaaaagaagaaagaacctgatgctaagcagcttggcgtaactagatct

SEQ ID NO: 349 - DNA, 350 bp, unidentified
  source 1..350: mol_type = unassigned DNA
ggccaaagcatgcatgccacccaagaaatcccgaggtttcagagctatgctggaaacagcatagcaagttgaaataaggctagtccgttatcaacttgaaaaagtggcaccgagtcggtgcctgctttcctctcgggatttcttgggaaaaaaaaagctgccatcagtcggcgtggactgtagaacactgccaatgccggtcccaagcccggataaaagtggagggtacagtccacgcttttttttgcactggtttccgcctccgacctgtggctggcctgttttcctctcgggatttcttgggtggcctggccttccaaacagctaagcagcttggcgtaactagatct

SEQ ID NO: 350 - DNA, 350 bp, unidentified
  source 1..350: mol_type = unassigned DNA
ggccaaagcatgcatgccacccaagaaatcccgaggtttcagagctatgctggaaacagcatagcaagttgaaataaggctagtccgttatcaacttgaaaaagtggcaccgagtcggtgctggcctgctttcctctcgggatttcttgggaaaaaaaaagctgccatcagtcggcgtggactgtagaacactgccaatgccggtcccaagcccggataaaagtggagggtacagtccacgctttttttctggtttccgcctccgacctgtggctggcctgttttcctctcgggatttcttgggtggcctggccttcctcccggctaagcagcttggcgtaactagatct

SEQ ID NO: 351 - DNA, 350 bp, unidentified
  source 1..350: mol_type = unassigned DNA
ggccaaagcatgcatgccacccaagaaatcccgaggtttcagagctatgctggaaacagcatagcaagttgaaataaggctagtccgttatcaacttgaaaaagtggcaccgagtcggtgcggctggcctgctttcctctcgggatttcttgggaaaaaaaaagctgccatcagtcggcgtggactgtagaacactgccaatgccggtcccaagcccggataaaagtggagggtacagtccacgctttttttgtttccgcctccgacctgtggctggcctgttttcctctcgggatttcttgggtggcctggccttcacacaagctaagcagcttggcgtaactagatct

SEQ ID NO: 352 - DNA, 350 bp, unidentified
  source 1..350: mol_type = unassigned DNA
ggccaaagcatgcatgccacccaagaaatcccgaggtttcagagctatgctggaaacagcatagcaagttgaaataaggctagtccgttatcaacttgaaaaagtggcaccgagtcggtgccctgtggctggcctgctttcctctcgggatttcttgggaaaaaaaaagctgccatcagtcggcgtggactgtagaacactgccaatgccggtcccaagcccggataaaagtggagggtacagtccacgctttttttcgcctccgacctgtggctggcctgttttcctctcgggatttcttgggtggcctggccttcgcgtgcgctaagcagcttggcgtaactagatct

SEQ ID NO: 353 - DNA, 350 bp, unidentified
  source 1..350: mol_type = unassigned DNA
ggccaaagcatgcatggaaatcccgagaggaaaacgtttcagagctatgctggaaacagcatagcaagttgaaataaggctagtccgttatcaacttgaaaaagtggcaccgagtcggtgcctgctttcctctcgggataaaaaaaaagctgccatcagtcggcgtggactgtagaacactgccaatgccggtcccaagcccggataaaagtggagggtacagtccacgcttttttttcttttcatcccagcttgcactggtttccgcctccgacctgtggctggcctgttttcctctcgggatttcttgggtggccagggaagctaagcagcttggcgtaactagatct

SEQ ID NO: 354 - DNA, 350 bp, unidentified
  source 1..350: mol_type = unassigned DNA
ggccaaagcatgcatggaaatcccgagaggaaaacgtttcagagctatgctggaaacagcatagcaagttgaaataaggctagtccgttatcaacttgaaaaagtggcaccgagtcggtgctggcctgctttcctctcgggataaaaaaaaagctgccatcagtcggcgtggactgtagaacactgccaatgccggtcccaagcccggataaaagtggagggtacagtccacgctttttttttcatcccagcttgcactggtttccgcctccgacctgtggctggcctgttttcctctcgggatttcttgggtggccgctggagctaagcagcttggcgtaactagatct

SEQ ID NO: 355 - DNA, 350 bp, unidentified
  source 1..350: mol_type = unassigned DNA
ggccaaagcatgcatggaaatcccgagaggaaaacgtttcagagctatgctggaaacagcatagcaagttgaaataaggctagtccgttatcaacttgaaaaagtggcaccgagtcggtgcggctggcctgctttcctctcgggataaaaaaaaagctgccatcagtcggcgtggactgtagaacactgccaatgccggtcccaagcccggataaaagtggagggtacagtccacgctttttttatcccagcttgcactggtttccgcctccgacctgtggctggcctgttttcctctcgggatttcttgggtggcctctgtagctaagcagcttggcgtaactagatct

SEQ ID NO: 356 - DNA, 350 bp, unidentified
  source 1..350: mol_type = unassigned DNA
ggccaaagcatgcatggaaatcccgagaggaaaacgtttcagagctatgctggaaacagcatagcaagttgaaataaggctagtccgttatcaacttgaaaaagtggcaccgagtcggtgccctgtggctggcctgctttcctctcgggataaaaaaaaagctgccatcagtcggcgtggactgtagaacactgccaatgccggtcccaagcccggataaaagtggagggtacagtccacgctttttttagcttgcactggtttccgcctccgacctgtggctggcctgttttcctctcgggatttcttgggtggccgcaaccgctaagcagcttggcgtaactagatct

SEQ ID NO: 357 - DNA, 350 bp, unidentified
  source 1..350: mol_type = unassigned DNA
ggccaaagcatgcatgtccgcctccgacctgtggcgtttcagagctatgctggaaacagcatagcaagttgaaataaggctagtccgttatcaacttgaaaaagtggcaccgagtcggtgcaaagcaggccagccacaggtcggaggcaaaaaaaaagctgccatcagtcggcgtggactgtagaacactgccaatgccggtcccaagcccggataaaagtggagggtacagtccacgctttttttaaggccaggccacccaagaaatcccgagaggaaaacaggccagccacaggtcggaggcggaaaccagtgcaagacatgctaagcagcttggcgtaactagatct

SEQ ID NO: 358 - DNA, 350 bp, unidentified
  source 1..350: mol_type = unassigned DNA
ggccaaagcatgcatgtccgcctccgacctgtggcgtttcagagctatgctggaaacagcatagcaagttgaaataaggctagtccgttatcaacttgaaaaagtggcaccgagtcggtgccgagaggaaagcaggccagccacaggtcggaggcaaaaaaaaagctgccatcagtcggcgtggactgtagaacactgccaatgccggtcccaagcccggataaaagtggagggtacagtccacgctttttttggccacccaagaaatcccgagaggaaaacaggccagccacaggtcggaggcggaaaccagtgcagcttaagctaagcagcttggcgtaactagatct

SEQ ID NO: 359 - DNA, 350 bp, unidentified
  source 1..350: mol_type = unassigned DNA
ggccaaagcatgcatggctggcctgctttcctctcgtttcagagctatgctggaaacagcatagcaagttgaaataaggctagtccgttatcaacttgaaaaagtggcaccgagtcggtgctcccgagaggaaagcaggccaaaaaaaaagctgccatcagtcggcgtggactgtagaacactgccaatgccggtcccaagcccggataaaagtggagggtacagtccacgcttttttttactgtgtgcagtggaagactcggaaggccaggccacccaagaaatcctgagaggaaagcaggccagccacaggtcggcgatctgctaagcagcttggcgtaactagatct

SEQ ID NO: 360 - DNA, 350 bp, unidentified
  source 1..350: mol_type = unassigned DNA
ggccaaagcatgcatgttccagcccctctattacagtttcagagctatgctggaaacagcatagcaagttgaaataaggctagtccgttatcaacttgaaaaagtggcaccgagtcggtgcccacgtaatagaggggctgaaaaaaaaagctgccatcagtcggcgtggactgtagaacactgccaatgccggtcccaagcccggataaaagtggagggtacagtccacgctttttttgtcaccacctcaccttactttctccttggcatcattaaaactctctgccatgtaatagaggggctggaactccgtgacaactagggctaagcagcttggcgtaactagatct

SEQ ID NO: 361 - DNA, 350 bp, unidentified
  source 1..350: mol_type = unassigned DNA
ggccaaagcatgcatgttccagcccctctattacagtttcagagctatgctggaaacagcatagcaagttgaaataaggctagtccgttatcaacttgaaaaagtggcaccgagtcggtgctctgccacgtaatagaggggctgaaaaaaaaagctgccatcagtcggcgtggactgtagaacactgccaatgccggtcccaagcccggataaaagtggagggtacagtccacgctttttttccacctcaccttactttctccttggcatcattaaaactctctgccatgtaatagaggggctggaactccgtgacacacgtagctaagcagcttggcgtaactagatct

SEQ ID NO: 362 - DNA, 350 bp, unidentified
  source 1..350: mol_type = unassigned DNA
ggccaaagcatgcatgttccagcccctctattacagtttcagagctatgctggaaacagcatagcaagttgaaataaggctagtccgttatcaacttgaaaaagtggcaccgagtcggtgcctctctgccacgtaatagaggggctgaaaaaaaaagctgccatcagtcggcgtggactgtagaacactgccaatgccggtcccaagcccggataaaagtggagggtacagtccacgctttttttcctcaccttactttctccttggcatcattaaaactctctgccatgtaatagaggggctggaactccgtgacaagccgggctaagcagcttggcgtaactagatct

SEQ ID NO: 363 - DNA, 350 bp, unidentified
  source 1..350: mol_type = unassigned DNA
ggccaaagcatgcatgttccagcccctctattacagtttcagagctatgctggaaacagcatagcaagttgaaataaggctagtccgttatcaacttgaaaaagtggcaccgagtcggtgctaaaactctctgccacgtaatagaggggctgaaaaaaaaagctgccatcagtcggcgtggactgtagaacactgccaatgccggtcccaagcccggataaaagtggagggtacagtccacgctttttttccttactttctccttggcatcattaaaactctctgccatgtaatagaggggctggaactccgtgacaatgtcggctaagcagcttggcgtaactagatct

SEQ ID NO: 364 - DNA, 350 bp, unidentified
  source 1..350: mol_type = unassigned DNA
ggccaaagcatgcatgagctggaggacagtactcagtttcagagctatgctggaaacagcatagcaagttgaaataaggctagtccgttatcaacttgaaaaagtggcaccgagtcggtgcaccgtgagtactgtcctccaaaaaaaaaagctgccatcagtcggcgtggactgtagaacactgccaatgccggtcccaagcccggataaaagtggagggtacagtccacgctttttttcacacagtacatcagacatggatccaagcccatgtatacccccgaaccatgagtactgtcctccagctaccagttgcctctgttgctaagcagcttggcgtaactagatct

SEQ ID NO: 365 - DNA, 350 bp, unidentified
  source 1..350: mol_type = unassigned DNA
ggccaaagcatgcatgagctggaggacagtactcagtttcagagctatgctggaaacagcatagcaagttgaaataaggctagtccgttatcaacttgaaaaagtggcaccgagtcggtgccccccgaaccgtgagtactgtcctccaaaaaaaaaagctgccatcagtcggcgtggactgtagaacactgccaatgccggtcccaagcccggataaaagtggagggtacagtccacgcttttttttacatcagacatggatccaagcccatgtatacccccgaaccatgagtactgtcctccagctaccagttgcccattaagctaagcagcttggcgtaactagatct

SEQ ID NO: 366 - DNA, 350 bp, unidentified
  source 1..350: mol_type = unassigned DNA
ggccaaagcatgcatgaggaccgcagtggacacgcgtttcagagctatgctggaaacagcatagcaagttgaaataaggctagtccgttatcaacttgaaaaagtggcaccgagtcggtgcagcatgtccactgcggtaaaaaaaaagctgccatcagtcggcgtggactgtagaacactgccaatgccggtcccaagcccggataaaagtggagggtacagtccacgctttttttctgcctgtacctgaggccctaaaaagccagagacctcactcccggggagccagcgtgtccactgcggtcctggaaaacccagcttctgctaagcagcttggcgtaactagatct

SEQ ID NO: 367 - DNA, 350 bp, unidentified
  source 1..350: mol_type = unassigned DNA
ggccaaagcatgcatgaggaccgcagtggacacgcgtttcagagctatgctggaaacagcatagcaagttgaaataaggctagtccgttatcaacttgaaaaagtggcaccgagtcggtgcagccagcatgtccactgcggtaaaaaaaaagctgccatcagtcggcgtggactgtagaacactgccaatgccggtcccaagcccggataaaagtggagggtacagtccacgctttttttctgtacctgaggccctaaaaagccagagacctcactcccggggagccagcgtgtccactgcggtcctggaaaacccagaatccgctaagcagcttggcgtaactagatct

SEQ ID NO: 368 - DNA, 350 bp, unidentified
  source 1..350: mol_type = unassigned DNA
ggccaaagcatgcatgaggaccgcagtggacacgcgtttcagagctatgctggaaacagcatagcaagttgaaataaggctagtccgttatcaacttgaaaaagtggcaccgagtcggtgcgggagccagcatgtccactgcggtaaaaaaaaagctgccatcagtcggcgtggactgtagaacactgccaatgccggtcccaagcccggataaaagtggagggtacagtccacgcttttttttacctgaggccctaaaaagccagagacctcactcccggggagccagcgtgtccactgcggtcctggaaaacccagtctaggctaagcagcttggcgtaactagatct

SEQ ID NO: 369 - DNA, 350 bp, unidentified
  source 1..350: mol_type = unassigned DNA
ggccaaagcatgcatgaggaccgcagtggacacgcgtttcagagctatgctggaaacagcatagcaagttgaaataaggctagtccgttatcaacttgaaaaagtggcaccgagtcggtgctcccggggagccagcatgtccactgcggtaaaaaaaaagctgccatcagtcggcgtggactgtagaacactgccaatgccggtcccaagcccggataaaagtggagggtacagtccacgctttttttgaggccctaaaaagccagagacctcactcccggggagccagcgtgtccactgcggtcctggaaaacccaatgagagctaagcagcttggcgtaactagatct

SEQ ID NO: 370 - DNA, 350 bp, unidentified
  source 1..350: mol_type = unassigned DNA
ggccaaagcatgcatgagccagagacctcactcccgtttcagagctatgctggaaacagcatagcaagttgaaataaggctagtccgttatcaacttgaaaaagtggcaccgagtcggtgcgtggacatgctggctccccgggagtgaggtctctgaaaaaaaaagctgccatcagtcggcgtggactgtagaacactgccaatgccggtcccaagcccggataaaagtggagggtacagtccacgctttttttttttccaggaccgcagtggacacgctggctccccgggagtgaggtctctggctttttagggccagacctgctaagcagcttggcgtaactagatct

SEQ ID NO: 371 - DNA, 350 bp, unidentified
  source 1..350: mol_type = unassigned DNA
ggccaaagcatgcatgagccagagacctcactcccgtttcagagctatgctggaaacagcatagcaagttgaaataaggctagtccgttatcaacttgaaaaagtggcaccgagtcggtgcgcagtggacatgctggctccccgggagtgaggtctctgaaaaaaaaagctgccatcagtcggcgtggactgtagaacactgccaatgccggtcccaagcccggataaaagtggagggtacagtccacgcttttttttccaggaccgcagtggacacgctggctccccgggagtgaggtctctggctttttagggcctacttcgctaagcagcttggcgtaactagatct

SEQ ID NO: 372 - DNA, 350 bp, unidentified
  source 1..350: mol_type = unassigned DNA
ggccaaagcatgcatggccagagacctcactcccggtttcagagctatgctggaaacagcatagcaagttgaaataaggctagtccgttatcaacttgaaaaagtggcaccgagtcggtgcacatgctggctccccgggagtgaggtctctaaaaaaaaagctgccatcagtcggcgtggactgtagaacactgccaatgccggtcccaagcccggataaaagtggagggtacagtccacgctttttttcctgggttttccaggaccgcagtggacacgctggctccccgggagtgaggtctctggctttttagggcctgtcagctaagcagcttggcgtaactagatct

SEQ ID NO: 373 - DNA, 350 bp, unidentified
  source 1..350: mol_type = unassigned DNA
ggccaaagcatgcatggccagagacctcactcccggtttcagagctatgctggaaacagcatagcaagttgaaataaggctagtccgttatcaacttgaaaaagtggcaccgagtcggtgcgtggacatgctggctccccgggagtgaggtctctaaaaaaaaagctgccatcagtcggcgtggactgtagaacactgccaatgccggtcccaagcccggataaaagtggagggtacagtccacgctttttttggttttccaggaccgcagtggacacgctggctccccgggagtgaggtctctggctttttagggcaagcgcgctaagcagcttggcgtaactagatct

SEQ ID NO: 374 - DNA, 350 bp, unidentified
  source 1..350: mol_type = unassigned DNA
ggccaaagcatgcatggccagagacctcactcccggtttcagagctatgctggaaacagcatagcaagttgaaataaggctagtccgttatcaacttgaaaaagtggcaccgagtcggtgcggaccgcagtggacatgctggctccccgggagtgaggtctctaaaaaaaaagctgccatcagtcggcgtggactgtagaacactgccaatgccggtcccaagcccggataaaagtggagggtacagtccacgctttttttaggaccgcagtggacacgctggctccccgggagtgaggtctctggctttttagggcgatacagctaagcagcttggcgtaactagatct

SEQ ID NO: 375 - DNA, 350 bp, unidentified
  source 1..350: mol_type = unassigned DNA
ggccaaagcatgcatgggcgtcatctctacgctgtgtttcagagctatgctggaaacagcatagcaagttgaaataaggctagtccgttatcaacttgaaaaagtggcaccgagtcggtgccctgcagcgtagagatgacaaaaaaaaagctgccatcagtcggcgtggactgtagaacactgccaatgccggtcccaagcccggataaaagtggagggtacagtccacgctttttttccagtgcctgggacctaggggaggagaagcaggggggtgatgtcttgcctacagcgtagagatgacgccagtggccaccttatctgctaagcagcttggcgtaactagatct

SEQ ID NO: 376 - DNA, 350 bp, unidentified
  source 1..350: mol_type = unassigned DNA
ggccaaagcatgcatgggcgtcatctctacgctgtgtttcagagctatgctggaaacagcatagcaagttgaaataaggctagtccgttatcaacttgaaaaagtggcaccgagtcggtgccttgcctgcagcgtagagatgacaaaaaaaaagctgccatcagtcggcgtggactgtagaacactgccaatgccggtcccaagcccggataaaagtggagggtacagtccacgcttttttttgcctgggacctaggggaggagaagcaggggggtgatgtcttgcctacagcgtagagatgacgccagtggccaccatccgtgctaagcagcttggcgtaactagatct

SEQ ID NO: 377 - DNA, 350 bp, unidentified
  source 1..350: mol_type = unassigned DNA
ggccaaagcatgcatgggcgtcatctctacgctgtgtttcagagctatgctggaaacagcatagcaagttgaaataaggctagtccgttatcaacttgaaaaagtggcaccgagtcggtgctgtcttgcctgcagcgtagagatgacaaaaaaaaagctgccatcagtcggcgtggactgtagaacactgccaatgccggtcccaagcccggataaaagtggagggtacagtccacgctttttttctgggacctaggggaggagaagcaggggggtgatgtcttgcctacagcgtagagatgacgccagtggccacctgacctgctaagcagcttggcgtaactagatct

SEQ ID NO: 378 - DNA, 350 bp, unidentified
  source 1..350: mol_type = unassigned DNA
ggccaaagcatgcatgggcgtcatctctacgctgtgtttcagagctatgctggaaacagcatagcaagttgaaataaggctagtccgttatcaacttgaaaaagtggcaccgagtcggtgcggtgatgtcttgcctgcagcgtagagatgacaaaaaaaaagctgccatcagtcggcgtggactgtagaacactgccaatgccggtcccaagcccggataaaagtggagggtacagtccacgctttttttacctaggggaggagaagcaggggggtgatgtcttgcctacagcgtagagatgacgccagtggccaccaaggtcgctaagcagcttggcgtaactagatct

SEQ ID NO: 379 - DNA, 350 bp, unidentified
  source 1..350: mol_type = unassigned DNA
ggccaaagcatgcatggagagttttaatgatgccagtttcagagctatgctggaaacagcatagcaagttgaaataaggctagtccgttatcaacttgaaaaagtggcaccgagtcggtgcccttactttctccttggcatcattaaaactaaaaaaaaagctgccatcagtcggcgtggactgtagaacactgccaatgccggtcccaagcccggataaaagtggagggtacagtccacgctttttttgtggctcacctttgtcaccacctcacctaactttctccttggcatcattaaaactctctgccacgtaaaccgatgctaagcagcttggcgtaactagatct

SEQ ID NO: 380 - DNA, 350 bp, unidentified
  source 1..350: mol_type = unassigned DNA
ggccaaagcatgcatggagagttttaatgatgccagtttcagagctatgctggaaacagcatagcaagttgaaataaggctagtccgttatcaacttgaaaaagtggcaccgagtcggtgcctcaccttactttctccttggcatcattaaaactaaaaaaaaagctgccatcagtcggcgtggactgtagaacactgccaatgccggtcccaagcccggataaaagtggagggtacagtccacgctttttttctcacctttgtcaccacctcacctaactttctccttggcatcattaaaactctctgccacgtaactcgtggctaagcagcttggcgtaactagatct

SEQ ID NO: 381 - DNA, 350 bp, unidentified
  source 1..350: mol_type = unassigned DNA
ggccaaagcatgcatggagagttttaatgatgccagtttcagagctatgctggaaacagcatagcaagttgaaataaggctagtccgttatcaacttgaaaaagtggcaccgagtcggtgccacctcaccttactttctccttggcatcattaaaactaaaaaaaaagctgccatcagtcggcgtggactgtagaacactgccaatgccggtcccaagcccggataaaagtggagggtacagtccacgctttttttacctttgtcaccacctcacctaactttctccttggcatcattaaaactctctgccacgtaagagttggctaagcagcttggcgtaactagatct

SEQ ID NO: 382 - DNA, 350 bp, unidentified
  source 1..350: mol_type = unassigned DNA
ggccaaagcatgcatggagagttttaatgatgccagtttcagagctatgctggaaacagcatagcaagttgaaataaggctagtccgttatcaacttgaaaaagtggcaccgagtcggtgcgtcaccacctcaccttactttctccttggcatcattaaaactaaaaaaaaagctgccatcagtcggcgtggactgtagaacactgccaatgccggtcccaagcccggataaaagtggagggtacagtccacgcttttttttgtcaccacctcacctaactttctccttggcatcattaaaactctctgccacgtaacctcctgctaagcagcttggcgtaactagatct

SEQ ID NO: 383 - DNA, 350 bp, unidentified
  source 1..350: mol_type = unassigned DNA
ggccaaagcatgcatgtgatgccaaggagaaagttgtttcagagctatgctggaaacagcatagcaagttgaaataaggctagtccgttatcaacttgaaaaagtggcaccgagtcggtgcccttactttctccttggcaaaaaaaaaagctgccatcagtcggcgtggactgtagaacactgccaatgccggtcccaagcccggataaaagtggagggtacagtccacgctttttttagtcaggaggcccccagagctagtggctcacctttgtcaccacctcacctaactttctccttggcatcattaaaactcttatctggctaagcagcttggcgtaactagatct

SEQ ID NO: 384 - DNA, 350 bp, unidentified
  source 1..350: mol_type = unassigned DNA
ggccaaagcatgcatgtgatgccaaggagaaagttgtttcagagctatgctggaaacagcatagcaagttgaaataaggctagtccgttatcaacttgaaaaagtggcaccgagtcggtgccacctcaccttactttctccttggcaaaaaaaaaagctgccatcagtcggcgtggactgtagaacactgccaatgccggtcccaagcccggataaaagtggagggtacagtccacgctttttttaggcccccagagctagtggctcacctttgtcaccacctcacctaactttctccttggcatcattaaaactctagataagctaagcagcttggcgtaactagatct

SEQ ID NO: 385 - DNA, 350 bp, unidentified
  source 1..350: mol_type = unassigned DNA
ggccaaagcatgcatgtgatgccaaggagaaagttgtttcagagctatgctggaaacagcatagcaagttgaaataaggctagtccgttatcaacttgaaaaagtggcaccgagtcggtgcgtcaccacctcaccttactttctccttggcaaaaaaaaaagctgccatcagtcggcgtggactgtagaacactgccaatgccggtcccaagcccggataaaagtggagggtacagtccacgctttttttcccagagctagtggctcacctttgtcaccacctcacctaactttctccttggcatcattaaaactctcaacgggctaagcagcttggcgtaactagatct

SEQ ID NO: 386 - DNA, 350 bp, unidentified
  source 1..350: mol_type = unassigned DNA
ggccaaagcatgcatgaggtgcacccagagagacagtttcagagctatgctggaaacagcatagcaagttgaaataaggctagtccgttatcaacttgaaaaagtggcaccgagtcggtgcttgcctctctgggtgcaaaaaaaaaagctgccatcagtcggcgtggactgtagaacactgccaatgccggtcccaagcccggataaaagtggagggtacagtccacgctttttttattacaggaacagaacaggttctattttcccccaattacaggaaattggccttgtctctctgggtgcacctgatgaatacactattcgctaagcagcttggcgtaactagatct

SEQ ID NO: 387 - DNA, 350 bp, unidentified
  source 1..350: mol_type = unassigned DNA
ggccaaagcatgcatgaggtgcacccagagagacagtttcagagctatgctggaaacagcatagcaagttgaaataaggctagtccgttatcaacttgaaaaagtggcaccgagtcggtgcggccttgcctctctgggtgcaaaaaaaaaagctgccatcagtcggcgtggactgtagaacactgccaatgccggtcccaagcccggataaaagtggagggtacagtccacgctttttttcaggaacagaacaggttctattttcccccaattacaggaaattggccttgtctctctgggtgcacctgatgaatacaattttcgctaagcagcttggcgtaactagatct

SEQ ID NO: 388 - DNA, 350 bp, unidentified
  source 1..350: mol_type = unassigned DNA
ggccaaagcatgcatgaggtgcacccagagagacagtttcagagctatgctggaaacagcatagcaagttgaaataaggctagtccgttatcaacttgaaaaagtggcaccgagtcggtgcattggccttgcctctctgggtgcaaaaaaaaaagctgccatcagtcggcgtggactgtagaacactgccaatgccggtcccaagcccggataaaagtggagggtacagtccacgctttttttgaacagaacaggttctattttcccccaattacaggaaattggccttgtctctctgggtgcacctgatgaatacaattaatgctaagcagcttggcgtaactagatct

SEQ ID NO: 389 - DNA, 350 bp, unidentified
  source 1..350: mol_type = unassigned DNA
ggccaaagcatgcatgaggtgcacccagagagacagtttcagagctatgctggaaacagcatagcaagttgaaataaggctagtccgttatcaacttgaaaaagtggcaccgagtcggtgcaggaaattggccttgcctctctgggtgcaaaaaaaaaagctgccatcagtcggcgtggactgtagaacactgccaatgccggtcccaagcccggataaaagtggagggtacagtccacgctttttttgaacaggttctattttcccccaattacaggaaattggccttgtctctctgggtgcacctgatgaatacaaatagtgctaagcagcttggcgtaactagatct

SEQ ID NO: 390 - DNA, 350 bp, unidentified
  source 1..350: mol_type = unassigned DNA
ggccaaagcatgcatgtcccccaattacaggaaatgtttcagagctatgctggaaacagcatagcaagttgaaataaggctagtccgttatcaacttgaaaaagtggcaccgagtcggtgcgaggcaaggccaatttcctgtaattgggaaaaaaaaagctgccatcagtcggcgtggactgtagaacactgccaatgccggtcccaagcccggataaaagtggagggtacagtccacgcttttttttttcaatgtattcatcaggtgcacccagagagacaaggccaatttcctgtaattgggggaaaatagaacccgttcggctaagcagcttggcgtaactagatct

SEQ ID NO: 391 - DNA, 350 bp, unidentified
  source 1..350: mol_type = unassigned DNA
ggccaaagcatgcatgtcccccaattacaggaaatgtttcagagctatgctggaaacagcatagcaagttgaaataaggctagtccgttatcaacttgaaaaagtggcaccgagtcggtgccagagaggcaaggccaatttcctgtaattgggaaaaaaaaagctgccatcagtcggcgtggactgtagaacactgccaatgccggtcccaagcccggataaaagtggagggtacagtccacgctttttttaatgtattcatcaggtgcacccagagagacaaggccaatttcctgtaattgggggaaaatagaaccgactccgctaagcagcttggcgtaactagatct

SEQ ID NO: 392 - DNA, 350 bp, unidentified
  source 1..350: mol_type = unassigned DNA
ggccaaagcatgcatgtcccccaattacaggaaatgtttcagagctatgctggaaacagcatagcaagttgaaataaggctagtccgttatcaacttgaaaaagtggcaccgagtcggtgcacccagagaggcaaggccaatttcctgtaattgggaaaaaaaaagctgccatcagtcggcgtggactgtagaacactgccaatgccggtcccaagcccggataaaagtggagggtacagtccacgctttttttgtattcatcaggtgcacccagagagacaaggccaatttcctgtaattgggggaaaatagaacctcctttgctaagcagcttggcgtaactagatct

SEQ ID NO: 393 - DNA, 350 bp, unidentified
  source 1..350: mol_type = unassigned DNA
ggccaaagcatgcatgtcccccaattacaggaaatgtttcagagctatgctggaaacagcatagcaagttgaaataaggctagtccgttatcaacttgaaaaagtggcaccgagtcggtgcggtgcacccagagaggcaaggccaatttcctgtaattgggaaaaaaaaagctgccatcagtcggcgtggactgtagaacactgccaatgccggtcccaagcccggataaaagtggagggtacagtccacgctttttttcatcaggtgcacccagagagacaaggccaatttcctgtaattgggggaaaatagaaccgctaaagctaagcagcttggcgtaactagatct

SEQ ID NO: 394 - DNA, 350 bp, unidentified
  source 1..350: mol_type = unassigned DNA
ggccaaagcatgcatgaaaaaatccattccttatcgtttcagagctatgctggaaacagcatagcaagttgaaataaggctagtccgttatcaacttgaaaaagtggcaccgagtcggtgccaggtaaggaatggatttaaaaaaaaagctgccatcagtcggcgtggactgtagaacactgccaatgccggtcccaagcccggataaaagtggagggtacagtccacgctttttttgttgggacatgtgcccttgttttcagatcgcagctttgcccagttttcccagataaggaatggattttttagccttctagccatcagctaagcagcttggcgtaactagatct

SEQ ID NO: 395 - DNA, 350 bp, unidentified
  source 1..350: mol_type = unassigned DNA
ggccaaagcatgcatgaaaaaatccattccttatcgtttcagagctatgctggaaacagcatagcaagttgaaataaggctagtccgttatcaacttgaaaaagtggcaccgagtcggtgcttcccaggtaaggaatggatttaaaaaaaaagctgccatcagtcggcgtggactgtagaacactgccaatgccggtcccaagcccggataaaagtggagggtacagtccacgctttttttggacatgtgcccttgttttcagatcgcagctttgcccagttttcccagataaggaatggattttttagccttctaggggtgagctaagcagcttggcgtaactagatct

SEQ ID NO: 396 - DNA, 350 bp, unidentified
  source 1..350: mol_type = unassigned DNA
ggccaaagcatgcatgaaaaaatccattccttatcgtttcagagctatgctggaaacagcatagcaagttgaaataaggctagtccgttatcaacttgaaaaagtggcaccgagtcggtgcgttttcccaggtaaggaatggatttaaaaaaaaagctgccatcagtcggcgtggactgtagaacactgccaatgccggtcccaagcccggataaaagtggagggtacagtccacgctttttttcatgtgcccttgttttcagatcgcagctttgcccagttttcccagataaggaatggattttttagccttctagcatcacgctaagcagcttggcgtaactagatct

SEQ ID NO: 397 - DNA, 350 bp, unidentified
  source 1..350: mol_type = unassigned DNA
ggccaaagcatgcatgaaaaaatccattccttatcgtttcagagctatgctggaaacagcatagcaagttgaaataaggctagtccgttatcaacttgaaaaagtggcaccgagtcggtgcgcccagttttcccaggtaaggaatggatttaaaaaaaaagctgccatcagtcggcgtggactgtagaacactgccaatgccggtcccaagcccggataaaagtggagggtacagtccacgctttttttgcccttgttttcagatcgcagctttgcccagttttcccagataaggaatggattttttagccttctaggtcctcgctaagcagcttggcgtaactagatct

SEQ ID NO: 398 - DNA, 350 bp, unidentified
  source 1..350: mol_type = unassigned DNA
ggccaaagcatgcatgaaaaatccattccttatctgtttcagagctatgctggaaacagcatagcaagttgaaataaggctagtccgttatcaacttgaaaaagtggcaccgagtcggtgccaggtaaggaatggattaaaaaaaaagctgccatcagtcggcgtggactgtagaacactgccaatgccggtcccaagcccggataaaagtggagggtacagtccacgctttttttctgttgggacatgtgcccttgttttcagatcgcagctttgcccagttttcccagataaggaatggattttttagccttctattcgaagctaagcagcttggcgtaactagatct

SEQ ID NO: 399 - DNA, 350 bp, unidentified
  source 1..350: mol_type = unassigned DNA
ggccaaagcatgcatgaaaaatccattccttatctgtttcagagctatgctggaaacagcatagcaagttgaaataaggctagtccgttatcaacttgaaaaagtggcaccgagtcggtgcttcccaggtaaggaatggattaaaaaaaaagctgccatcagtcggcgtggactgtagaacactgccaatgccggtcccaagcccggataaaagtggagggtacagtccacgcttttttttgggacatgtgcccttgttttcagatcgcagctttgcccagttttcccagataaggaatggattttttagccttctagccagggctaagcagcttggcgtaactagatct

SEQ ID NO: 400 - DNA, 350 bp, unidentified
  source 1..350: mol_type = unassigned DNA
ggccaaagcatgcatgaaaaatccattccttatctgtttcagagctatgctggaaacagcatagcaagttgaaataaggctagtccgttatcaacttgaaaaagtggcaccgagtcggtgcgttttcccaggtaaggaatggattaaaaaaaaagctgccatcagtcggcgtggactgtagaacactgccaatgccggtcccaagcccggataaaagtggagggtacagtccacgctttttttgacatgtgcccttgttttcagatcgcagctttgcccagttttcccagataaggaatggattttttagccttctagagggagctaagcagcttggcgtaactagatct

SEQ ID NO: 401 - DNA, 350 bp, unidentified
  source 1..350: mol_type = unassigned DNA
ggccaaagcatgcatgaaaaatccattccttatctgtttcagagctatgctggaaacagcatagcaagttgaaataaggctagtccgttatcaacttgaaaaagtggcaccgagtcggtgcgcccagttttcccaggtaaggaatggattaaaaaaaaagctgccatcagtcggcgtggactgtagaacactgccaatgccggtcccaagcccggataaaagtggagggtacagtccacgctttttttgtgcccttgttttcagatcgcagctttgcccagttttcccagataaggaatggattttttagccttctaatcaaggctaagcagcttggcgtaactagatct

SEQ ID NO: 402 - DNA, 350 bp, unidentified
  source 1..350: mol_type = unassigned DNA
ggccaaagcatgcatgtgcccagttttcccagatagtttcagagctatgctggaaacagcatagcaagttgaaataaggctagtccgttatcaacttgaaaaagtggcaccgagtcggtgcttacctgggaaaactggaaaaaaaaagctgccatcagtcggcgtggactgtagaacactgccaatgccggtcccaagcccggataaaagtggagggtacagtccacgcttttttttgagaaattcaggtcacagacctataactagaaggctaaaaaatccattccttatctgggaaaactgggcaaagctgcgattccagtgctaagcagcttggcgtaactagatct

SEQ ID NO: 403 - DNA, 350 bp, unidentified
  source 1..350: mol_type = unassigned DNA
ggccaaagcatgcatgtgcccagttttcccagatagtttcagagctatgctggaaacagcatagcaagttgaaataaggctagtccgttatcaacttgaaaaagtggcaccgagtcggtgcttccttacctgggaaaactggaaaaaaaaagctgccatcagtcggcgtggactgtagaacactgccaatgccggtcccaagcccggataaaagtggagggtacagtccacgctttttttaaattcaggtcacagacctataactagaaggctaaaaaatccattccttatctgggaaaactgggcaaagctgcgatgagccggctaagcagcttggcgtaactagatct

SEQ ID NO: 404 - DNA, 350 bp, unidentified
  source 1..350: mol_type = unassigned DNA
ggccaaagcatgcatgtgcccagttttcccagatagtttcagagctatgctggaaacagcatagcaagttgaaataaggctagtccgttatcaacttgaaaaagtggcaccgagtcggtgcccattccttacctgggaaaactggaaaaaaaaagctgccatcagtcggcgtggactgtagaacactgccaatgccggtcccaagcccggataaaagtggagggtacagtccacgctttttttttcaggtcacagacctataactagaaggctaaaaaatccattccttatctgggaaaactgggcaaagctgcgatggtgttgctaagcagcttggcgtaactagatct

SEQ ID NO: 405 - DNA, 350 bp, unidentified
  source 1..350: mol_type = unassigned DNA
ggccaaagcatgcatgtgcccagttttcccagatagtttcagagctatgctggaaacagcatagcaagttgaaataaggctagtccgttatcaacttgaaaaagtggcaccgagtcggtgcaaaatccattccttacctgggaaaactggaaaaaaaaagctgccatcagtcggcgtggactgtagaacactgccaatgccggtcccaagcccggataaaagtggagggtacagtccacgctttttttgtcacagacctataactagaaggctaaaaaatccattccttatctgggaaaactgggcaaagctgcgattaggacgctaagcagcttggcgtaactagatct

SEQ ID NO: 406 - DNA, 350 bp, unidentified
  source 1..350: mol_type = unassigned DNA
ggccaaagcatgcatggggcttggatccatgtctggtttcagagctatgctggaaacagcatagcaagttgaaataaggctagtccgttatcaacttgaaaaagtggcaccgagtcggtgctacatcagacatggatccaagaaaaaaaaagctgccatcagtcggcgtggactgtagaacactgccaatgccggtcccaagcccggataaaagtggagggtacagtccacgcttttttttttcttgggtggcctggccttccgagtcttccactgcacacagtacctcagacatggatccaagcccatgtatacccgtcagcgctaagcagcttggcgtaactagatct

SEQ ID NO: 407 - DNA, 350 bp, unidentified
  source 1..350: mol_type = unassigned DNA
ggccaaagcatgcatggggcttggatccatgtctggtttcagagctatgctggaaacagcatagcaagttgaaataaggctagtccgttatcaacttgaaaaagtggcaccgagtcggtgcacagtacatcagacatggatccaagaaaaaaaaagctgccatcagtcggcgtggactgtagaacactgccaatgccggtcccaagcccggataaaagtggagggtacagtccacgctttttttttgggtggcctggccttccgagtcttccactgcacacagtacctcagacatggatccaagcccatgtatacccttccctgctaagcagcttggcgtaactagatct

SEQ ID NO: 408 - DNA, 350 bp, unidentified
  source 1..350: mol_type = unassigned DNA
ggccaaagcatgcatggggcttggatccatgtctggtttcagagctatgctggaaacagcatagcaagttgaaataaggctagtccgttatcaacttgaaaaagtggcaccgagtcggtgccacacagtacatcagacatggatccaagaaaaaaaaagctgccatcagtcggcgtggactgtagaacactgccaatgccggtcccaagcccggataaaagtggagggtacagtccacgctttttttggtggcctggccttccgagtcttccactgcacacagtacctcagacatggatccaagcccatgtatacccgtattcgctaagcagcttggcgtaactagatct

SEQ ID NO: 409 - DNA, 350 bp, unidentified
  source 1..350: mol_type = unassigned DNA
ggccaaagcatgcatggggcttggatccatgtctggtttcagagctatgctggaaacagcatagcaagttgaaataaggctagtccgttatcaacttgaaaaagtggcaccgagtcggtgccactgcacacagtacatcagacatggatccaagaaaaaaaaagctgccatcagtcggcgtggactgtagaacactgccaatgccggtcccaagcccggataaaagtggagggtacagtccacgctttttttcctggccttccgagtcttccactgcacacagtacctcagacatggatccaagcccatgtatacccgttgccgctaagcagcttggcgtaactagatct

SEQ ID NO: 410 - DNA, 350 bp, unidentified
  source 1..350: mol_type = unassigned DNA
ggccaaagcatgcatgggaagccacagtacttttcgtttcagagctatgctggaaacagcatagcaagttgaaataaggctagtccgttatcaacttgaaaaagtggcaccgagtcggtgcttcttgaaaagtactgtggctaaaaaaaaagctgccatcagtcggcgtggactgtagaacactgccaatgccggtcccaagcccggataaaagtggagggtacagtccacgcttttttttataaaacccatgcttgctatgagtacaatcacatttttccacttcctgaaaagtactgtggcttccatgaagataaccttacgctaagcagcttggcgtaactagatct

SEQ ID NO: 411 - DNA, 350 bp, unidentified
  source 1..350: mol_type = unassigned DNA
ggccaaagcatgcatgggaagccacagtacttttcgtttcagagctatgctggaaacagcatagcaagttgaaataaggctagtccgttatcaacttgaaaaagtggcaccgagtcggtgcccacttcttgaaaagtactgtggctaaaaaaaaagctgccatcagtcggcgtggactgtagaacactgccaatgccggtcccaagcccggataaaagtggagggtacagtccacgctttttttaaacccatgcttgctatgagtacaatcacatttttccacttcctgaaaagtactgtggcttccatgaagataagctcgggctaagcagcttggcgtaactagatct

SEQ ID NO: 412 - DNA, 350 bp, unidentified
  source 1..350: mol_type = unassigned DNA
ggccaaagcatgcatgggaagccacagtacttttcgtttcagagctatgctggaaacagcatagcaagttgaaataaggctagtccgttatcaacttgaaaaagtggcaccgagtcggtgctttccacttcttgaaaagtactgtggctaaaaaaaaagctgccatcagtcggcgtggactgtagaacactgccaatgccggtcccaagcccggataaaagtggagggtacagtccacgctttttttcccatgcttgctatgagtacaatcacatttttccacttcctgaaaagtactgtggcttccatgaagataaccattggctaagcagcttggcgtaactagatct

SEQ ID NO: 413 - DNA, 350 bp, unidentified
  source 1..350: mol_type = unassigned DNA
ggccaaagcatgcatgggaagccacagtacttttcgtttcagagctatgctggaaacagcatagcaagttgaaataaggctagtccgttatcaacttgaaaaagtggcaccgagtcggtgcacatttttccacttcttgaaaagtactgtggctaaaaaaaaagctgccatcagtcggcgtggactgtagaacactgccaatgccggtcccaagcccggataaaagtggagggtacagtccacgctttttttgcttgctatgagtacaatcacatttttccacttcctgaaaagtactgtggcttccatgaagataacgaagcgctaagcagcttggcgtaactagatct

SEQ ID NO: 414 - DNA, 350 bp, unidentified
  source 1..350: mol_type = unassigned DNA
ggccaaagcatgcatgcattaggaccatggtggatgtttcagagctatgctggaaacagcatagcaagttgaaataaggctagtccgttatcaacttgaaaaagtggcaccgagtcggtgctcacgcctctgcccgatccaccatggtcctaaaaaaaaaagctgccatcagtcggcgtggactgtagaacactgccaatgccggtcccaagcccggataaaagtggagggtacagtccacgctttttttccccctgggtgtccctgtaggactcatgcctctgcccgatccaccatggtcctaatgagctcccagaacctgtgctaagcagcttggcgtaactagatct

SEQ ID NO: 415 - DNA, 350 bp, unidentified
  source 1..350: mol_type = unassigned DNA
ggccaaagcatgcatgcattaggaccatggtggatgtttcagagctatgctggaaacagcatagcaagttgaaataaggctagtccgttatcaacttgaaaaagtggcaccgagtcggtgcggactcacgcctctgcccgatccaccatggtcctaaaaaaaaaagctgccatcagtcggcgtggactgtagaacactgccaatgccggtcccaagcccggataaaagtggagggtacagtccacgctttttttctgggtgtccctgtaggactcatgcctctgcccgatccaccatggtcctaatgagctcccagatatttcgctaagcagcttggcgtaactagatct

SEQ ID NO: 416 - DNA, 350 bp, unidentified
  source 1..350: mol_type = unassigned DNA
ggccaaagcatgcatgcattaggaccatggtggatgtttcagagctatgctggaaacagcatagcaagttgaaataaggctagtccgttatcaacttgaaaaagtggcaccgagtcggtgcgtaggactcacgcctctgcccgatccaccatggtcctaaaaaaaaaagctgccatcagtcggcgtggactgtagaacactgccaatgccggtcccaagcccggataaaagtggagggtacagtccacgctttttttggtgtccctgtaggactcatgcctctgcccgatccaccatggtcctaatgagctcccagaggcgaggctaagcagcttggcgtaactagatct

SEQ ID NO: 417 - DNA, 350 bp, unidentified
  source 1..350: mol_type = unassigned DNA
ggccaaagcatgcatgattaggaccatggtggatcgtttcagagctatgctggaaacagcatagcaagttgaaataaggctagtccgttatcaacttgaaaaagtggcaccgagtcggtgctcacgcctctgcccgatccaccatggtcctaaaaaaaaagctgccatcagtcggcgtggactgtagaacactgccaatgccggtcccaagcccggataaaagtggagggtacagtccacgcttttttttgccccctgggtgtccctgtaggactcatgcctctgcccgatccaccatggtcctaatgagctcccaggcggtagctaagcagcttggcgtaactagatct

SEQ ID NO: 418 - DNA, 350 bp, unidentified
  source 1..350: mol_type = unassigned DNA
ggccaaagcatgcatgattaggaccatggtggatcgtttcagagctatgctggaaacagcatagcaagttgaaataaggctagtccgttatcaacttgaaaaagtggcaccgagtcggtgcggactcacgcctctgcccgatccaccatggtcctaaaaaaaaagctgccatcagtcggcgtggactgtagaacactgccaatgccggtcccaagcccggataaaagtggagggtacagtccacgctttttttccctgggtgtccctgtaggactcatgcctctgcccgatccaccatggtcctaatgagctcccagagagaggctaagcagcttggcgtaactagatct

SEQ ID NO: 419 - DNA, 350 bp, unidentified
  source 1..350: mol_type = unassigned DNA
ggccaaagcatgcatgattaggaccatggtggatcgtttcagagctatgctggaaacagcatagcaagttgaaataaggctagtccgttatcaacttgaaaaagtggcaccgagtcggtgcgtaggactcacgcctctgcccgatccaccatggtcctaaaaaaaaagctgccatcagtcggcgtggactgtagaacactgccaatgccggtcccaagcccggataaaagtggagggtacagtccacgcttttttttgggtgtccctgtaggactcatgcctctgcccgatccaccatggtcctaatgagctcccagggttgagctaagcagcttggcgtaactagatct

SEQ ID NO: 420 - DNA, 350 bp, unidentified
  source 1..350: mol_type = unassigned DNA
ggccaaagcatgcatgattaggaccatggtggatcgtttcagagctatgctggaaacagcatagcaagttgaaataaggctagtccgttatcaacttgaaaaagtggcaccgagtcggtgctccctgtaggactcacgcctctgcccgatccaccatggtcctaaaaaaaaagctgccatcagtcggcgtggactgtagaacactgccaatgccggtcccaagcccggataaaagtggagggtacagtccacgctttttttgtccctgtaggactcatgcctctgcccgatccaccatggtcctaatgagctcccagcactaggctaagcagcttggcgtaactagatct

SEQ ID NO: 421 - DNA, 350 bp, unidentified
  source 1..350: mol_type = unassigned DNA
ggccaaagcatgcatggccccctgggtgtccctgtgtttcagagctatgctggaaacagcatagcaagttgaaataaggctagtccgttatcaacttgaaaaagtggcaccgagtcggtgcggcgtgagtcctacagggacacccagggaaaaaaaaagctgccatcagtcggcgtggactgtagaacactgccaatgccggtcccaagcccggataaaagtggagggtacagtccacgctttttttctcattaggaccatggtggatcgggcagaggcatgagtcctacagggacacccagggggcagacagaggttcttttgctaagcagcttggcgtaactagatct

SEQ ID NO: 422 - DNA, 350 bp, unidentified
  source 1..350: mol_type = unassigned DNA
ggccaaagcatgcatggccccctgggtgtccctgtgtttcagagctatgctggaaacagcatagcaagttgaaataaggctagtccgttatcaacttgaaaaagtggcaccgagtcggtgccagaggcgtgagtcctacagggacacccagggaaaaaaaaagctgccatcagtcggcgtggactgtagaacactgccaatgccggtcccaagcccggataaaagtggagggtacagtccacgctttttttttaggaccatggtggatcgggcagaggcatgagtcctacagggacacccagggggcagacagaggttaagcggctaagcagcttggcgtaactagatct

SEQ ID NO: 423 - DNA, 350 bp, unidentified
  source 1..350: mol_type = unassigned DNA
ggccaaagcatgcatggccccctgggtgtccctgtgtttcagagctatgctggaaacagcatagcaagttgaaataaggctagtccgttatcaacttgaaaaagtggcaccgagtcggtgcgggcagaggcgtgagtcctacagggacacccagggaaaaaaaaagctgccatcagtcggcgtggactgtagaacactgccaatgccggtcccaagcccggataaaagtggagggtacagtccacgctttttttggaccatggtggatcgggcagaggcatgagtcctacagggacacccagggggcagacagaggtttgaccgctaagcagcttggcgtaactagatct

SEQ ID NO: 424 - DNA, 350 bp, unidentified
  source 1..350: mol_type = unassigned DNA
ggccaaagcatgcatggccccctgggtgtccctgtgtttcagagctatgctggaaacagcatagcaagttgaaataaggctagtccgttatcaacttgaaaaagtggcaccgagtcggtgcggatcgggcagaggcgtgagtcctacagggacacccagggaaaaaaaaagctgccatcagtcggcgtggactgtagaacactgccaatgccggtcccaagcccggataaaagtggagggtacagtccacgctttttttatggtggatcgggcagaggcatgagtcctacagggacacccagggggcagacagaggttaccctgctaagcagcttggcgtaactagatct

SEQ ID NO: 425 - DNA, 350 bp, unidentified
  source 1..350: mol_type = unassigned DNA
ggccaaagcatgcatgaaagtattgcgcttatttggtttcagagctatgctggaaacagcatagcaagttgaaataaggctagtccgttatcaacttgaaaaagtggcaccgagtcggtgcgcactgacctcaaataagcgcaatacaaaaaaaaagctgccatcagtcggcgtggactgtagaacactgccaatgccggtcccaagcccggataaaagtggagggtacagtccacgctttttttgctacgacattatccaagacaaacatgattgtagcagtgacctcaaataagcgcaatactttggccaatgcacaagtagctaagcagcttggcgtaactagatct

SEQ ID NO: 426 - DNA, 350 bp, unidentified
  source 1..350: mol_type = unassigned DNA
ggccaaagcatgcatgaaagtattgcgcttatttggtttcagagctatgctggaaacagcatagcaagttgaaataaggctagtccgttatcaacttgaaaaagtggcaccgagtcggtgctgtagcactgacctcaaataagcgcaatacaaaaaaaaagctgccatcagtcggcgtggactgtagaacactgccaatgccggtcccaagcccggataaaagtggagggtacagtccacgctttttttcgacattatccaagacaaacatgattgtagcagtgacctcaaataagcgcaatactttggccaatgcagagtgtgctaagcagcttggcgtaactagatct

SEQ ID NO: 427 - DNA, 350 bp, unidentified
  source 1..350: mol_type = unassigned DNA
ggccaaagcatgcatgaaagtattgcgcttatttggtttcagagctatgctggaaacagcatagcaagttgaaataaggctagtccgttatcaacttgaaaaagtggcaccgagtcggtgcgattgtagcactgacctcaaataagcgcaatacaaaaaaaaagctgccatcagtcggcgtggactgtagaacactgccaatgccggtcccaagcccggataaaagtggagggtacagtccacgctttttttcattatccaagacaaacatgattgtagcagtgacctcaaataagcgcaatactttggccaatgcatagaatgctaagcagcttggcgtaactagatct

SEQ ID NO: 428 - DNA, 350 bp, unidentified
  source 1..350: mol_type = unassigned DNA
ggccaaagcatgcatgaaagtattgcgcttatttggtttcagagctatgctggaaacagcatagcaagttgaaataaggctagtccgttatcaacttgaaaaagtggcaccgagtcggtgcaacatgattgtagcactgacctcaaataagcgcaatacaaaaaaaaagctgccatcagtcggcgtggactgtagaacactgccaatgccggtcccaagcccggataaaagtggagggtacagtccacgcttttttttccaagacaaacatgattgtagcagtgacctcaaataagcgcaatactttggccaatgcattgcaggctaagcagcttggcgtaactagatct

SEQ ID NO: 429 - DNA, 350 bp, unidentified
  source 1..350: mol_type = unassigned DNA
ggccaaagcatgcatgagcaaggcagacttactcggtttcagagctatgctggaaacagcatagcaagttgaaataaggctagtccgttatcaacttgaaaaagtggcaccgagtcggtgcctaccgccagtaagtctgccttaaaaaaaaagctgccatcagtcggcgtggactgtagaacactgccaatgccggtcccaagcccggataaaagtggagggtacagtccacgcttttttttaccgtgcaagacggaagcagtttgctgacattgcctacaactaccgcgagtaagtctgccttgcttgttgaggggggtgccgctaagcagcttggcgtaactagatct

SEQ ID NO: 430 - DNA, 350 bp, unidentified
  source 1..350: mol_type = unassigned DNA
ggccaaagcatgcatgagcaaggcagacttactcggtttcagagctatgctggaaacagcatagcaagttgaaataaggctagtccgttatcaacttgaaaaagtggcaccgagtcggtgccaactaccgccagtaagtctgccttaaaaaaaaagctgccatcagtcggcgtggactgtagaacactgccaatgccggtcccaagcccggataaaagtggagggtacagtccacgctttttttcgtgcaagacggaagcagtttgctgacattgcctacaactaccgcgagtaagtctgccttgcttgttgagggggtgggtgctaagcagcttggcgtaactagatct

SEQ ID NO: 431 - DNA, 350 bp, unidentified
  source 1..350: mol_type = unassigned DNA
ggccaaagcatgcatgagcaaggcagacttactcggtttcagagctatgctggaaacagcatagcaagttgaaataaggctagtccgttatcaacttgaaaaagtggcaccgagtcggtgcgcctacaactaccgccagtaagtctgccttaaaaaaaaagctgccatcagtcggcgtggactgtagaacactgccaatgccggtcccaagcccggataaaagtggagggtacagtccacgctttttttaagacggaagcagtttgctgacattgcctacaactaccgcgagtaagtctgccttgcttgttgaggggtacgatgctaagcagcttggcgtaactagatct

SEQ ID NO: 432 - DNA, 350 bp, unidentified
  source 1..350: mol_type = unassigned DNA
ggccaaagcatgcatgccaaggagaaagtaagctggtttcagagctatgctggaaacagcatagcaagttgaaataaggctagtccgttatcaacttgaaaaagtggcaccgagtcggtgctcaccttactttctcctaaaaaaaaagctgccatcagtcggcgtggactgtagaacactgccaatgccggtcccaagcccggataaaagtggagggtacagtccacgcttttttttggcaccagtcaggaggcccccagagctagtggctcacctttgtcaccacctcagcttactttctccttggcatcattaaacccggcgctaagcagcttggcgtaactagatct

SEQ ID NO: 433 - DNA, 350 bp, unidentified
  source 1..350: mol_type = unassigned DNA
ggccaaagcatgcatgccaaggagaaagtaagctggtttcagagctatgctggaaacagcatagcaagttgaaataaggctagtccgttatcaacttgaaaaagtggcaccgagtcggtgccacctcaccttactttctcctaaaaaaaaagctgccatcagtcggcgtggactgtagaacactgccaatgccggtcccaagcccggataaaagtggagggtacagtccacgctttttttaccagtcaggaggcccccagagctagtggctcacctttgtcaccacctcagcttactttctccttggcatcattaaacgacgagctaagcagcttggcgtaactagatct

SEQ ID NO: 434 - DNA, 350 bp, unidentified
  source 1..350: mol_type = unassigned DNA
ggccaaagcatgcatgccaaggagaaagtaagctggtttcagagctatgctggaaacagcatagcaagttgaaataaggctagtccgttatcaacttgaaaaagtggcaccgagtcggtgccaccacctcaccttactttctcctaaaaaaaaagctgccatcagtcggcgtggactgtagaacactgccaatgccggtcccaagcccggataaaagtggagggtacagtccacgctttttttagtcaggaggcccccagagctagtggctcacctttgtcaccacctcagcttactttctccttggcatcattaaatggcgcgctaagcagcttggcgtaactagatct

SEQ ID NO: 435 - DNA, 350 bp, unidentified
  source 1..350: mol_type = unassigned DNA
ggccaaagcatgcatgccaaggagaaagtaagctggtttcagagctatgctggaaacagcatagcaagttgaaataaggctagtccgttatcaacttgaaaaagtggcaccgagtcggtgctttgtcaccacctcaccttactttctcctaaaaaaaaagctgccatcagtcggcgtggactgtagaacactgccaatgccggtcccaagcccggataaaagtggagggtacagtccacgctttttttggaggcccccagagctagtggctcacctttgtcaccacctcagcttactttctccttggcatcattaaacttgacgctaagcagcttggcgtaactagatct

SEQ ID NO: 436 - DNA, 350 bp, unidentified
  source 1..350: mol_type = unassigned DNA
ggccaaagcatgcatgggtcactcccaagaacccggtttcagagctatgctggaaacagcatagcaagttgaaataaggctagtccgttatcaacttgaaaaagtggcaccgagtcggtgctccacgggttcttgggagtgaaaaaaaaagctgccatcagtcggcgtggactgtagaacactgccaatgccggtcccaagcccggataaaagtggagggtacagtccacgcttttttttggagggtgggatttacctgatgtgcatgaggttctcatccatgctccgcgggttcttgggagtgaccgggatgggaaatgtgtgctaagcagcttggcgtaactagatct

SEQ ID NO: 437 - DNA, 350 bp, unidentified
  source 1..350: mol_type = unassigned DNA
ggccaaagcatgcatgggtcactcccaagaacccggtttcagagctatgctggaaacagcatagcaagttgaaataaggctagtccgttatcaacttgaaaaagtggcaccgagtcggtgcatgctccacgggttcttgggagtgaaaaaaaaagctgccatcagtcggcgtggactgtagaacactgccaatgccggtcccaagcccggataaaagtggagggtacagtccacgctttttttgggtgggatttacctgatgtgcatgaggttctcatccatgctccgcgggttcttgggagtgaccgggatgggaagttgcggctaagcagcttggcgtaactagatct

SEQ ID NO: 438 - DNA, 350 bp, unidentified
  source 1..350: mol_type = unassigned DNA
ggccaaagcatgcatgggtcactcccaagaacccggtttcagagctatgctggaaacagcatagcaagttgaaataaggctagtccgttatcaacttgaaaaagtggcaccgagtcggtgctccatgctccacgggttcttgggagtgaaaaaaaaagctgccatcagtcggcgtggactgtagaacactgccaatgccggtcccaagcccggataaaagtggagggtacagtccacgcttttttttgggatttacctgatgtgcatgaggttctcatccatgctccgcgggttcttgggagtgaccgggatgggaatattcggctaagcagcttggcgtaactagatct

SEQ ID NO: 439 - DNA, 350 bp, unidentified
  source 1..350: mol_type = unassigned DNA
ggccaaagcatgcatgggtcactcccaagaacccggtttcagagctatgctggaaacagcatagcaagttgaaataaggctagtccgttatcaacttgaaaaagtggcaccgagtcggtgctctcatccatgctccacgggttcttgggagtgaaaaaaaaagctgccatcagtcggcgtggactgtagaacactgccaatgccggtcccaagcccggataaaagtggagggtacagtccacgcttttttttttacctgatgtgcatgaggttctcatccatgctccgcgggttcttgggagtgaccgggatgggaactctcggctaagcagcttggcgtaactagatct

SEQ ID NO: 440 - DNA, 350 bp, unidentified
  source 1..350: mol_type = unassigned DNA
ggccaaagcatgcatgggttctcatccatgctccggtttcagagctatgctggaaacagcatagcaagttgaaataaggctagtccgttatcaacttgaaaaagtggcaccgagtcggtgcccgtggagcatggatgagaaaaaaaaaagctgccatcagtcggcgtggactgtagaacactgccaatgccggtcccaagcccggataaaagtggagggtacagtccacgctttttttctttcctgcagcaacacgggattcccatcccggtcactcccaagaacccgcggagcatggatgagaacctcatgcacatgctagtgctaagcagcttggcgtaactagatct

SEQ ID NO: 441 - DNA, 350 bp, unidentified
  source 1..350: mol_type = unassigned DNA
ggccaaagcatgcatgggttctcatccatgctccggtttcagagctatgctggaaacagcatagcaagttgaaataaggctagtccgttatcaacttgaaaaagtggcaccgagtcggtgcgaacccgtggagcatggatgagaaaaaaaaaagctgccatcagtcggcgtggactgtagaacactgccaatgccggtcccaagcccggataaaagtggagggtacagtccacgctttttttcctgcagcaacacgggattcccatcccggtcactcccaagaacccgcggagcatggatgagaacctcatgcacataaccgtgctaagcagcttggcgtaactagatct

SEQ ID NO: 442 - DNA, 350 bp, unidentified
  source 1..350: mol_type = unassigned DNA
ggccaaagcatgcatgggttctcatccatgctccggtttcagagctatgctggaaacagcatagcaagttgaaataaggctagtccgttatcaacttgaaaaagtggcaccgagtcggtgccaagaacccgtggagcatggatgagaaaaaaaaaagctgccatcagtcggcgtggactgtagaacactgccaatgccggtcccaagcccggataaaagtggagggtacagtccacgctttttttgcagcaacacgggattcccatcccggtcactcccaagaacccgcggagcatggatgagaacctcatgcacattggtaggctaagcagcttggcgtaactagatct

SEQ ID NO: 443 - DNA, 350 bp, unidentified
  source 1..350: mol_type = unassigned DNA
ggccaaagcatgcatgggttctcatccatgctccggtttcagagctatgctggaaacagcatagcaagttgaaataaggctagtccgttatcaacttgaaaaagtggcaccgagtcggtgcactcccaagaacccgtggagcatggatgagaaaaaaaaaagctgccatcagtcggcgtggactgtagaacactgccaatgccggtcccaagcccggataaaagtggagggtacagtccacgctttttttaacacgggattcccatcccggtcactcccaagaacccgcggagcatggatgagaacctcatgcacatgcttttgctaagcagcttggcgtaactagatct

SEQ ID NO: 444 - DNA, 350 bp, unidentified
  source 1..350: mol_type = unassigned DNA
ggccaaagcatgcatggttctcatccatgctccgcgtttcagagctatgctggaaacagcatagcaagttgaaataaggctagtccgttatcaacttgaaaaagtggcaccgagtcggtgcccgtggagcatggatgagaaaaaaaaagctgccatcagtcggcgtggactgtagaacactgccaatgccggtcccaagcccggataaaagtggagggtacagtccacgctttttttctctttcctgcagcaacacgggattcccatcccggtcactcccaagaacccgcggagcatggatgagaacctcatgcacaccgtacgctaagcagcttggcgtaactagatct

SEQ ID NO: 445 - DNA, 350 bp, unidentified
  source 1..350: mol_type = unassigned DNA
ggccaaagcatgcatggttctcatccatgctccgcgtttcagagctatgctggaaacagcatagcaagttgaaataaggctagtccgttatcaacttgaaaaagtggcaccgagtcggtgcgaacccgtggagcatggatgagaaaaaaaaagctgccatcagtcggcgtggactgtagaacactgccaatgccggtcccaagcccggataaaagtggagggtacagtccacgctttttttttcctgcagcaacacgggattcccatcccggtcactcccaagaacccgcggagcatggatgagaacctcatgcacactagttgctaagcagcttggcgtaactagatct

SEQ ID NO: 446 - DNA, 350 bp, unidentified
  source 1..350: mol_type = unassigned DNA
ggccaaagcatgcatggttctcatccatgctccgcgtttcagagctatgctggaaacagcatagcaagttgaaataaggctagtccgttatcaacttgaaaaagtggcaccgagtcggtgccaagaacccgtggagcatggatgagaaaaaaaaagctgccatcagtcggcgtggactgtagaacactgccaatgccggtcccaagcccggataaaagtggagggtacagtccacgctttttttctgcagcaacacgggattcccatcccggtcactcccaagaacccgcggagcatggatgagaacctcatgcacaatacacgctaagcagcttggcgtaactagatct

SEQ ID NO: 447 - DNA, 350 bp, unidentified
  source 1..350: mol_type = unassigned DNA
ggccaaagcatgcatggttctcatccatgctccgcgtttcagagctatgctggaaacagcatagcaagttgaaataaggctagtccgttatcaacttgaaaaagtggcaccgagtcggtgcactcccaagaacccgtggagcatggatgagaaaaaaaaagctgccatcagtcggcgtggactgtagaacactgccaatgccggtcccaagcccggataaaagtggagggtacagtccacgctttttttgcaacacgggattcccatcccggtcactcccaagaacccgcggagcatggatgagaacctcatgcacatgctcagctaagcagcttggcgtaactagatct

SEQ ID NO: 448 - DNA, 350 bp, unidentified
  source 1..350: mol_type = unassigned DNA
ggccaaagcatgcatgttaaattcttcctcctttagtttcagagctatgctggaaacagcatagcaagttgaaataaggctagtccgttatcaacttgaaaaagtggcaccgagtcggtgctccggctcataaccctaaaggaggaagaattaaaaaaaaagctgccatcagtcggcgtggactgtagaacactgccaatgccggtcccaagcccggataaaagtggagggtacagtccacgctttttttctgccaacttggttacactagcatccagctcataaccctaaaggaggaagaatttaaatcttatttacgcggtgctaagcagcttggcgtaactagatct

SEQ ID NO: 449 - DNA, 350 bp, unidentified
  source 1..350: mol_type = unassigned DNA
ggccaaagcatgcatgttaaattcttcctcctttagtttcagagctatgctggaaacagcatagcaagttgaaataaggctagtccgttatcaacttgaaaaagtggcaccgagtcggtgcgttacactagcatccggctcataaccctaaaggaggaagaattaaaaaaaaagctgccatcagtcggcgtggactgtagaacactgccaatgccggtcccaagcccggataaaagtggagggtacagtccacgctttttttttacactagcatccagctcataaccctaaaggaggaagaatttaaatcttatttaaatgtggctaagcagcttggcgtaactagatct

SEQ ID NO: 450 - DNA, 350 bp, unidentified
  source 1..350: mol_type = unassigned DNA
ggccaaagcatgcatgctcctttagggttatgagcgtttcagagctatgctggaaacagcatagcaagttgaaataaggctagtccgttatcaacttgaaaaagtggcaccgagtcggtgcagcatccggctcataaccctaaagaaaaaaaaagctgccatcagtcggcgtggactgtagaacactgccaatgccggtcccaagcccggataaaagtggagggtacagtccacgctttttttcctctttggcatactgctctgccaacttggttacactagcatccagctcataaccctaaaggaggaagaatttacaaatcgctaagcagcttggcgtaactagatct

SEQ ID NO: 451 - DNA, 350 bp, unidentified
  source 1..350: mol_type = unassigned DNA
ggccaaagcatgcatgctcctttagggttatgagcgtttcagagctatgctggaaacagcatagcaagttgaaataaggctagtccgttatcaacttgaaaaagtggcaccgagtcggtgcactagcatccggctcataaccctaaagaaaaaaaaagctgccatcagtcggcgtggactgtagaacactgccaatgccggtcccaagcccggataaaagtggagggtacagtccacgctttttttctttggcatactgctctgccaacttggttacactagcatccagctcataaccctaaaggaggaagaatttacgttaagctaagcagcttggcgtaactagatct

SEQ ID NO: 452 - DNA, 350 bp, unidentified
  source 1..350: mol_type = unassigned DNA
ggccaaagcatgcatgctcctttagggttatgagcgtttcagagctatgctggaaacagcatagcaagttgaaataaggctagtccgttatcaacttgaaaaagtggcaccgagtcggtgcgttacactagcatccggctcataaccctaaagaaaaaaaaagctgccatcagtcggcgtggactgtagaacactgccaatgccggtcccaagcccggataaaagtggagggtacagtccacgctttttttgcatactgctctgccaacttggttacactagcatccagctcataaccctaaaggaggaagaatttatcagcggctaagcagcttggcgtaactagatct

SEQ ID NO: 453 - DNA, 350 bp, unidentified
  source 1..350: mol_type = unassigned DNA
ggccaaagcatgcatgagcagccgctcagagtctcgtttcagagctatgctggaaacagcatagcaagttgaaataaggctagtccgttatcaacttgaaaaagtggcaccgagtcggtgcacccgagactctgagcggctaaaaaaaaagctgccatcagtcggcgtggactgtagaacactgccaatgccggtcccaagcccggataaaagtggagggtacagtccacgctttttttctgaccctcctgcccctggcttcccacagccacgccgtggcactgacctgagactctgagcggctgctggaggtgcggagtttcgctaagcagcttggcgtaactagatct

SEQ ID NO: 454 - DNA, 350 bp, unidentified
  source 1..350: mol_type = unassigned DNA
ggccaaagcatgcatgagcagccgctcagagtctcgtttcagagctatgctggaaacagcatagcaagttgaaataaggctagtccgttatcaacttgaaaaagtggcaccgagtcggtgcactgacccgagactctgagcggctaaaaaaaaagctgccatcagtcggcgtggactgtagaacactgccaatgccggtcccaagcccggataaaagtggagggtacagtccacgctttttttccctcctgcccctggcttcccacagccacgccgtggcactgacctgagactctgagcggctgctggaggtgcggtgcgaagctaagcagcttggcgtaactagatct

SEQ ID NO: 455 - DNA, 350 bp, unidentified
  source 1..350: mol_type = unassigned DNA
ggccaaagcatgcatgagcagccgctcagagtctcgtttcagagctatgctggaaacagcatagcaagttgaaataaggctagtccgttatcaacttgaaaaagtggcaccgagtcggtgcggcactgacccgagactctgagcggctaaaaaaaaagctgccatcagtcggcgtggactgtagaacactgccaatgccggtcccaagcccggataaaagtggagggtacagtccacgcttttttttcctgcccctggcttcccacagccacgccgtggcactgacctgagactctgagcggctgctggaggtgcggaatgaagctaagcagcttggcgtaactagatct

SEQ ID NO: 456 - DNA, 350 bp, unidentified
  source 1..350: mol_type = unassigned DNA
ggccaaagcatgcatgagcagccgctcagagtctcgtttcagagctatgctggaaacagcatagcaagttgaaataaggctagtccgttatcaacttgaaaaagtggcaccgagtcggtgcgccgtggcactgacccgagactctgagcggctaaaaaaaaagctgccatcagtcggcgtggactgtagaacactgccaatgccggtcccaagcccggataaaagtggagggtacagtccacgctttttttcccctggcttcccacagccacgccgtggcactgacctgagactctgagcggctgctggaggtgcggttggaggctaagcagcttggcgtaactagatct

SEQ ID NO: 457 - DNA, 350 bp, unidentified
  source 1..350: mol_type = unassigned DNA
ggccaaagcatgcatgtcctgtgtaccgtgcaagagtttcagagctatgctggaaacagcatagcaagttgaaataaggctagtccgttatcaacttgaaaaagtggcaccgagtcggtgctccgtcttgcacggtacacaaaaaaaaaagctgccatcagtcggcgtggactgtagaacactgccaatgccggtcccaagcccggataaaagtggagggtacagtccacgctttttttggcagacttactggcggtagttgtaggcaatgtcagcaaactgcttccatcttgcacggtacacaggatctttaaaacacggctgctaagcagcttggcgtaactagatct

SEQ ID NO: 458 - DNA, 350 bp, unidentified
  source 1..350: mol_type = unassigned DNA
ggccaaagcatgcatgtcctgtgtaccgtgcaagagtttcagagctatgctggaaacagcatagcaagttgaaataaggctagtccgttatcaacttgaaaaagtggcaccgagtcggtgctgcttccgtcttgcacggtacacaaaaaaaaaagctgccatcagtcggcgtggactgtagaacactgccaatgccggtcccaagcccggataaaagtggagggtacagtccacgctttttttgacttactggcggtagttgtaggcaatgtcagcaaactgcttccatcttgcacggtacacaggatctttaaaactccgcagctaagcagcttggcgtaactagatct

SEQ ID NO: 459 - DNA, 350 bp, unidentified
  source 1..350: mol_type = unassigned DNA
ggccaaagcatgcatgtcctgtgtaccgtgcaagagtttcagagctatgctggaaacagcatagcaagttgaaataaggctagtccgttatcaacttgaaaaagtggcaccgagtcggtgcaactgcttccgtcttgcacggtacacaaaaaaaaaagctgccatcagtcggcgtggactgtagaacactgccaatgccggtcccaagcccggataaaagtggagggtacagtccacgctttttttttactggcggtagttgtaggcaatgtcagcaaactgcttccatcttgcacggtacacaggatctttaaaaccttttcgctaagcagcttggcgtaactagatct

SEQ ID NO: 460 - DNA, 350 bp, unidentified
  source 1..350: mol_type = unassigned DNA
ggccaaagcatgcatgtcctgtgtaccgtgcaagagtttcagagctatgctggaaacagcatagcaagttgaaataaggctagtccgttatcaacttgaaaaagtggcaccgagtcggtgccagcaaactgcttccgtcttgcacggtacacaaaaaaaaaagctgccatcagtcggcgtggactgtagaacactgccaatgccggtcccaagcccggataaaagtggagggtacagtccacgctttttttggcggtagttgtaggcaatgtcagcaaactgcttccatcttgcacggtacacaggatctttaaaacgtgcgcgctaagcagcttggcgtaactagatct

SEQ ID NO: 461 - DNA, 350 bp, unidentified
  source 1..350: mol_type = unassigned DNA
ggccaaagcatgcatggggcagcccatccctcgaagtttcagagctatgctggaaacagcatagcaagttgaaataaggctagtccgttatcaacttgaaaaagtggcaccgagtcggtgcccactcgagggatgggctgaaaaaaaaagctgccatcagtcggcgtggactgtagaacactgccaatgccggtcccaagcccggataaaagtggagggtacagtccacgctttttttagtcttgaacactgtgccccatgttttcttttcttcctccatgtattccattcgagggatgggctgcccactagaataccgtctagctaagcagcttggcgtaactagatct

SEQ ID NO: 462 - DNA, 350 bp, unidentified
  source 1..350: mol_type = unassigned DNA
ggccaaagcatgcatggggcagcccatccctcgaagtttcagagctatgctggaaacagcatagcaagttgaaataaggctagtccgttatcaacttgaaaaagtggcaccgagtcggtgctattccactcgagggatgggctgaaaaaaaaagctgccatcagtcggcgtggactgtagaacactgccaatgccggtcccaagcccggataaaagtggagggtacagtccacgctttttttttgaacactgtgccccatgttttcttttcttcctccatgtattccattcgagggatgggctgcccactagaatacttctcagctaagcagcttggcgtaactagatct

SEQ ID NO: 463 - DNA, 350 bp, unidentified
  source 1..350: mol_type = unassigned DNA
ggccaaagcatgcatggggcagcccatccctcgaagtttcagagctatgctggaaacagcatagcaagttgaaataaggctagtccgttatcaacttgaaaaagtggcaccgagtcggtgcatgtattccactcgagggatgggctgaaaaaaaaagctgccatcagtcggcgtggactgtagaacactgccaatgccggtcccaagcccggataaaagtggagggtacagtccacgctttttttaacactgtgccccatgttttcttttcttcctccatgtattccattcgagggatgggctgcccactagaataccaacgcgctaagcagcttggcgtaactagatct

SEQ ID NO: 464 - DNA, 350 bp, unidentified
  source 1..350: mol_type = unassigned DNA
ggccaaagcatgcatggggcagcccatccctcgaagtttcagagctatgctggaaacagcatagcaagttgaaataaggctagtccgttatcaacttgaaaaagtggcaccgagtcggtgccctccatgtattccactcgagggatgggctgaaaaaaaaagctgccatcagtcggcgtggactgtagaacactgccaatgccggtcccaagcccggataaaagtggagggtacagtccacgcttttttttgtgccccatgttttcttttcttcctccatgtattccattcgagggatgggctgcccactagaatacatgagtgctaagcagcttggcgtaactagatct

SEQ ID NO: 465 - DNA, 350 bp, unidentified
  source 1..350: mol_type = unassigned DNA
ggccaaagcatgcatgttccttacctgggaaaactgtttcagagctatgctggaaacagcatagcaagttgaaataaggctagtccgttatcaacttgaaaaagtggcaccgagtcggtgcatcgcagctttgcccagttttcccaggtaagaaaaaaaaagctgccatcagtcggcgtggactgtagaacactgccaatgccggtcccaagcccggataaaagtggagggtacagtccacgctttttttgggacatgtgcccttgttttcagatcacagctttgcccagttttcccaggtaaggaatggattttttgcaacagctaagcagcttggcgtaactagatct

SEQ ID NO: 466 - DNA, 350 bp, unidentified
  source 1..350: mol_type = unassigned DNA
ggccaaagcatgcatgttccttacctgggaaaactgtttcagagctatgctggaaacagcatagcaagttgaaataaggctagtccgttatcaacttgaaaaagtggcaccgagtcggtgcttttcagatcgcagctttgcccagttttcccaggtaagaaaaaaaaagctgccatcagtcggcgtggactgtagaacactgccaatgccggtcccaagcccggataaaagtggagggtacagtccacgctttttttgtgcccttgttttcagatcacagctttgcccagttttcccaggtaaggaatggatttttttgacatgctaagcagcttggcgtaactagatct

SEQ ID NO: 467 - DNA, 350 bp, unidentified
  source 1..350: mol_type = unassigned DNA
ggccaaagcatgcatgttccttacctgggaaaactgtttcagagctatgctggaaacagcatagcaagttgaaataaggctagtccgttatcaacttgaaaaagtggcaccgagtcggtgcccttgttttcagatcgcagctttgcccagttttcccaggtaagaaaaaaaaagctgccatcagtcggcgtggactgtagaacactgccaatgccggtcccaagcccggataaaagtggagggtacagtccacgctttttttcttgttttcagatcacagctttgcccagttttcccaggtaaggaatggatttttttcgaccgctaagcagcttggcgtaactagatct

SEQ ID NO: 468 - DNA, 350 bp, unidentified
  source 1..350: mol_type = unassigned DNA
ggccaaagcatgcatgttgtactcatagcaagcatgtttcagagctatgctggaaacagcatagcaagttgaaataaggctagtccgttatcaacttgaaaaagtggcaccgagtcggtgcacccatgcttgctatgagtaaaaaaaaaagctgccatcagtcggcgtggactgtagaacactgccaatgccggtcccaagcccggataaaagtggagggtacagtccacgctttttttaaaacatggggcacagtgttcaagactctgaagtccttgtataaaacctatgcttgctatgagtacaatcacatttttgaggtagctaagcagcttggcgtaactagatct

SEQ ID NO: 469 - DNA, 350 bp, unidentified
  source 1..350: mol_type = unassigned DNA
ggccaaagcatgcatgttgtactcatagcaagcatgtttcagagctatgctggaaacagcatagcaagttgaaataaggctagtccgttatcaacttgaaaaagtggcaccgagtcggtgctaaaacccatgcttgctatgagtaaaaaaaaaagctgccatcagtcggcgtggactgtagaacactgccaatgccggtcccaagcccggataaaagtggagggtacagtccacgctttttttcatggggcacagtgttcaagactctgaagtccttgtataaaacctatgcttgctatgagtacaatcacatttttcccttagctaagcagcttggcgtaactagatct

SEQ ID NO: 470 - DNA, 350 bp, unidentified
  source 1..350: mol_type = unassigned DNA
ggccaaagcatgcatgttgtactcatagcaagcatgtttcagagctatgctggaaacagcatagcaagttgaaataaggctagtccgttatcaacttgaaaaagtggcaccgagtcggtgcgtataaaacccatgcttgctatgagtaaaaaaaaaagctgccatcagtcggcgtggactgtagaacactgccaatgccggtcccaagcccggataaaagtggagggtacagtccacgctttttttggggcacagtgttcaagactctgaagtccttgtataaaacctatgcttgctatgagtacaatcacattttttctggggctaagcagcttggcgtaactagatct

SEQ ID NO: 471 - DNA, 350 bp, unidentified
  source 1..350: mol_type = unassigned DNA
ggccaaagcatgcatgttgtactcatagcaagcatgtttcagagctatgctggaaacagcatagcaagttgaaataaggctagtccgttatcaacttgaaaaagtggcaccgagtcggtgctccttgtataaaacccatgcttgctatgagtaaaaaaaaaagctgccatcagtcggcgtggactgtagaacactgccaatgccggtcccaagcccggataaaagtggagggtacagtccacgctttttttacagtgttcaagactctgaagtccttgtataaaacctatgcttgctatgagtacaatcacatttttccgtttgctaagcagcttggcgtaactagatct

SEQ ID NO: 472 - DNA, 350 bp, unidentified
  source 1..350: mol_type = unassigned DNA
ggccaaagcatgcatgatggctttactttattttcgtttcagagctatgctggaaacagcatagcaagttgaaataaggctagtccgttatcaacttgaaaaagtggcaccgagtcggtgcgtgccctccagaaaataaagtaaagcaaaaaaaaagctgccatcagtcggcgtggactgtagaacactgccaatgccggtcccaagcccggataaaagtggagggtacagtccacgctttttttttttctttgtaggtgaaattggaatcctttgcagtgacctccagaaaataaagtaaagccatggacagaatgggtactgctaagcagcttggcgtaactagatct

SEQ ID NO: 473 - DNA, 350 bp, unidentified
  source 1..350: mol_type = unassigned DNA
ggccaaagcatgcatgatggctttactttattttcgtttcagagctatgctggaaacagcatagcaagttgaaataaggctagtccgttatcaacttgaaaaagtggcaccgagtcggtgctgcagtgccctccagaaaataaagtaaagcaaaaaaaaagctgccatcagtcggcgtggactgtagaacactgccaatgccggtcccaagcccggataaaagtggagggtacagtccacgctttttttctttgtaggtgaaattggaatcctttgcagtgacctccagaaaataaagtaaagccatggacagaatggatctggctaagcagcttggcgtaactagatct

SEQ ID NO: 474 - DNA, 350 bp, unidentified
  source 1..350: mol_type = unassigned DNA
ggccaaagcatgcatgatggctttactttattttcgtttcagagctatgctggaaacagcatagcaagttgaaataaggctagtccgttatcaacttgaaaaagtggcaccgagtcggtgcctttgcagtgccctccagaaaataaagtaaagcaaaaaaaaagctgccatcagtcggcgtggactgtagaacactgccaatgccggtcccaagcccggataaaagtggagggtacagtccacgcttttttttgtaggtgaaattggaatcctttgcagtgacctccagaaaataaagtaaagccatggacagaatggatcgggctaagcagcttggcgtaactagatct

SEQ ID NO: 475 - DNA, 350 bp, unidentified
  source 1..350: mol_type = unassigned DNA
ggccaaagcatgcatgatggctttactttattttcgtttcagagctatgctggaaacagcatagcaagttgaaataaggctagtccgttatcaacttgaaaaagtggcaccgagtcggtgcgaatcctttgcagtgccctccagaaaataaagtaaagcaaaaaaaaagctgccatcagtcggcgtggactgtagaacactgccaatgccggtcccaagcccggataaaagtggagggtacagtccacgctttttttgtgaaattggaatcctttgcagtgacctccagaaaataaagtaaagccatggacagaatgtctgccgctaagcagcttggcgtaactagatct

SEQ ID NO: 476 - DNA, 350 bp, unidentified
  source 1..350: mol_type = unassigned DNA
ggccaaagcatgcatggctttactttattttctgggtttcagagctatgctggaaacagcatagcaagttgaaataaggctagtccgttatcaacttgaaaaagtggcaccgagtcggtgcgtgccctccagaaaataaagtaaaaaaaaaaagctgccatcagtcggcgtggactgtagaacactgccaatgccggtcccaagcccggataaaagtggagggtacagtccacgcttttttttggtgtttttctttgtaggtgaaattggaatcctttgcagtgacctccagaaaataaagtaaagccatggacagaccaaaagctaagcagcttggcgtaactagatct

SEQ ID NO: 477 - DNA, 350 bp, unidentified
  source 1..350: mol_type = unassigned DNA
ggccaaagcatgcatggctttactttattttctgggtttcagagctatgctggaaacagcatagcaagttgaaataaggctagtccgttatcaacttgaaaaagtggcaccgagtcggtgctgcagtgccctccagaaaataaagtaaaaaaaaaaagctgccatcagtcggcgtggactgtagaacactgccaatgccggtcccaagcccggataaaagtggagggtacagtccacgctttttttgtttttctttgtaggtgaaattggaatcctttgcagtgacctccagaaaataaagtaaagccatggacagacccaatgctaagcagcttggcgtaactagatct

SEQ ID NO: 478 - DNA, 350 bp, unidentified
  source 1..350: mol_type = unassigned DNA
ggccaaagcatgcatggctttactttattttctgggtttcagagctatgctggaaacagcatagcaagttgaaataaggctagtccgttatcaacttgaaaaagtggcaccgagtcggtgcgaatcctttgcagtgccctccagaaaataaagtaaaaaaaaaaagctgccatcagtcggcgtggactgtagaacactgccaatgccggtcccaagcccggataaaagtggagggtacagtccacgctttttttttgtaggtgaaattggaatcctttgcagtgacctccagaaaataaagtaaagccatggacagatcacatgctaagcagcttggcgtaactagatct

SEQ ID NO: 479 - DNA, 350 bp, unidentified
  source 1..350: mol_type = unassigned DNA
ggccaaagcatgcatgtcgggggtatacatgggctgtttcagagctatgctggaaacagcatagcaagttgaaataaggctagtccgttatcaacttgaaaaagtggcaccgagtcggtgctacatcagacatggatccaagcccatgtataccccaaaaaaaaagctgccatcagtcggcgtggactgtagaacactgccaatgccggtcccaagcccggataaaagtggagggtacagtccacgctttttttttccactgcacacagtacatcaaacatggatccaagcccatgtatacccccgaaccgtgagtaggttgcgctaagcagcttggcgtaactagatct

SEQ ID NO: 480 - DNA, 350 bp, unidentified
  source 1..350: mol_type = unassigned DNA
ggccaaagcatgcatgactcatctttctttaaacggtttcagagctatgctggaaacagcatagcaagttgaaataaggctagtccgttatcaacttgaaaaagtggcaccgagtcggtgcccttctcgtttaaagaaagatgaaaaaaaaagctgccatcagtcggcgtggactgtagaacactgccaatgccggtcccaagcccggataaaagtggagggtacagtccacgcttttttttcttctaggagaatgatgtaaacctgacccacattgaatctagacctcgtttaaagaaagatgagtatgaatttttagagccgctaagcagcttggcgtaactagatct

SEQ ID NO: 481 - DNA, 350 bp, unidentified
  source 1..350: mol_type = unassigned DNA
ggccaaagcatgcatgactcatctttctttaaacggtttcagagctatgctggaaacagcatagcaagttgaaataaggctagtccgttatcaacttgaaaaagtggcaccgagtcggtgctagaccttctcgtttaaagaaagatgaaaaaaaaagctgccatcagtcggcgtggactgtagaacactgccaatgccggtcccaagcccggataaaagtggagggtacagtccacgctttttttctaggagaatgatgtaaacctgacccacattgaatctagacctcgtttaaagaaagatgagtatgaattttttattcagctaagcagcttggcgtaactagatct

SEQ ID NO: 482 - DNA, 350 bp, unidentified
  source 1..350: mol_type = unassigned DNA
ggccaaagcatgcatgactcatctttctttaaacggtttcagagctatgctggaaacagcatagcaagttgaaataaggctagtccgttatcaacttgaaaaagtggcaccgagtcggtgcatctagaccttctcgtttaaagaaagatgaaaaaaaaagctgccatcagtcggcgtggactgtagaacactgccaatgccggtcccaagcccggataaaagtggagggtacagtccacgctttttttggagaatgatgtaaacctgacccacattgaatctagacctcgtttaaagaaagatgagtatgaatttttcacgacgctaagcagcttggcgtaactagatct

SEQ ID NO: 483 - DNA, 350 bp, unidentified
  source 1..350: mol_type = unassigned DNA
ggccaaagcatgcatgactcatctttctttaaacggtttcagagctatgctggaaacagcatagcaagttgaaataaggctagtccgttatcaacttgaaaaagtggcaccgagtcggtgcattgaatctagaccttctcgtttaaagaaagatgaaaaaaaaagctgccatcagtcggcgtggactgtagaacactgccaatgccggtcccaagcccggataaaagtggagggtacagtccacgctttttttatgatgtaaacctgacccacattgaatctagacctcgtttaaagaaagatgagtatgaattttttcaaaagctaagcagcttggcgtaactagatct

SEQ ID NO: 484 - DNA, 350 bp, unidentified
  source 1..350: mol_type = unassigned DNA
ggccaaagcatgcatgacaagcaaggcagacttacgtttcagagctatgctggaaacagcatagcaagttgaaataaggctagtccgttatcaacttgaaaaagtggcaccgagtcggtgcactaccgccagtaagtctgccttgctaaaaaaaaagctgccatcagtcggcgtggactgtagaacactgccaatgccggtcccaagcccggataaaagtggagggtacagtccacgctttttttgcaagacggaagcagtttgctgacattgcctacaactccgccagtaagtctgccttgcttgttgaggggaagttggttgctaagcagcttggcgtaactagatct

SEQ ID NO: 485 - DNA, 350 bp, unidentified
  source 1..350: mol_type = unassigned DNA
ggccaaagcatgcatgacaagcaaggcagacttacgtttcagagctatgctggaaacagcatagcaagttgaaataaggctagtccgttatcaacttgaaaaagtggcaccgagtcggtgctacaactaccgccagtaagtctgccttgctaaaaaaaaagctgccatcagtcggcgtggactgtagaacactgccaatgccggtcccaagcccggataaaagtggagggtacagtccacgctttttttgacggaagcagtttgctgacattgcctacaactccgccagtaagtctgccttgcttgttgaggggaagttcggtgctaagcagcttggcgtaactagatct

SEQ ID NO: 486 - DNA, 350 bp, unidentified
  source 1..350: mol_type = unassigned DNA
ggccaaagcatgcatgagcaaggcagacttactgggtttcagagctatgctggaaacagcatagcaagttgaaataaggctagtccgttatcaacttgaaaaagtggcaccgagtcggtgcactaccgccagtaagtctgccttaaaaaaaaagctgccatcagtcggcgtggactgtagaacactgccaatgccggtcccaagcccggataaaagtggagggtacagtccacgcttttttttaccgtgcaagacggaagcagtttgctgacattgcctacaactccgccagtaagtctgccttgcttgttgaggggtatgtggctaagcagcttggcgtaactagatct

SEQ ID NO: 487 - DNA, 350 bp, unidentified
  source 1..350: mol_type = unassigned DNA
ggccaaagcatgcatgagcaaggcagacttactgggtttcagagctatgctggaaacagcatagcaagttgaaataaggctagtccgttatcaacttgaaaaagtggcaccgagtcggtgctacaactaccgccagtaagtctgccttaaaaaaaaagctgccatcagtcggcgtggactgtagaacactgccaatgccggtcccaagcccggataaaagtggagggtacagtccacgctttttttgtgcaagacggaagcagtttgctgacattgcctacaactccgccagtaagtctgccttgcttgttgaggggatgtgcgctaagcagcttggcgtaactagatct

SEQ ID NO: 488 - DNA, 350 bp, unidentified
  source 1..350: mol_type = unassigned DNA
ggccaaagcatgcatgccatccacccagggagagagtttcagagctatgctggaaacagcatagcaagttgaaataaggctagtccgttatcaacttgaaaaagtggcaccgagtcggtgcgccacagtaagtcccttctctccctgggtggaaaaaaaaaagctgccatcagtcggcgtggactgtagaacactgccaatgccggtcccaagcccggataaaagtggagggtacagtccacgctttttttcctgatgaatacattgaaaagctcgccgtaagtcccttctctccctgggtggatggtggagtgctagcaagagctaagcagcttggcgtaactagatct

SEQ ID NO: 489 - DNA, 350 bp, unidentified
  source 1..350: mol_type = unassigned DNA
ggccaaagcatgcatgccatccacccagggagagagtttcagagctatgctggaaacagcatagcaagttgaaataaggctagtccgttatcaacttgaaaaagtggcaccgagtcggtgcaaagctcgccacagtaagtcccttctctccctgggtggaaaaaaaaaagctgccatcagtcggcgtggactgtagaacactgccaatgccggtcccaagcccggataaaagtggagggtacagtccacgctttttttaatacattgaaaagctcgccgtaagtcccttctctccctgggtggatggtggagtgctagtagcagctaagcagcttggcgtaactagatct

SEQ ID NO: 490 - DNA, 350 bp, unidentified
  source 1..350: mol_type = unassigned DNA
ggccaaagcatgcatgccatccacccagggagagagtttcagagctatgctggaaacagcatagcaagttgaaataaggctagtccgttatcaacttgaaaaagtggcaccgagtcggtgcattgaaaagctcgccacagtaagtcccttctctccctgggtggaaaaaaaaaagctgccatcagtcggcgtggactgtagaacactgccaatgccggtcccaagcccggataaaagtggagggtacagtccacgctttttttattgaaaagctcgccgtaagtcccttctctccctgggtggatggtggagtgctatggttggctaagcagcttggcgtaactagatct

SEQ ID NO: 491 - DNA, 350 bp, unidentified
  source 1..350: mol_type = unassigned DNA
ggccaaagcatgcatgcatccacccagggagagaagtttcagagctatgctggaaacagcatagcaagttgaaataaggctagtccgttatcaacttgaaaaagtggcaccgagtcggtgcgccacagtaagtcccttctctccctgggtggaaaaaaaaagctgccatcagtcggcgtggactgtagaacactgccaatgccggtcccaagcccggataaaagtggagggtacagtccacgctttttttcacctgatgaatacattgaaaagctcgccgtaagtcccttctctccctgggtggatggtggagtgctcagtaagctaagcagcttggcgtaactagatct

SEQ ID NO: 492 - DNA, 350 bp, unidentified
  source 1..350: mol_type = unassigned DNA
ggccaaagcatgcatgcatccacccagggagagaagtttcagagctatgctggaaacagcatagcaagttgaaataaggctagtccgttatcaacttgaaaaagtggcaccgagtcggtgcgctcgccacagtaagtcccttctctccctgggtggaaaaaaaaagctgccatcagtcggcgtggactgtagaacactgccaatgccggtcccaagcccggataaaagtggagggtacagtccacgcttttttttgatgaatacattgaaaagctcgccgtaagtcccttctctccctgggtggatggtggagtgctggaggcgctaagcagcttggcgtaactagatct

SEQ ID NO: 493 - DNA, 350 bp, unidentified
  source 1..350: mol_type = unassigned DNA
ggccaaagcatgcatgcatccacccagggagagaagtttcagagctatgctggaaacagcatagcaagttgaaataaggctagtccgttatcaacttgaaaaagtggcaccgagtcggtgcaaagctcgccacagtaagtcccttctctccctgggtggaaaaaaaaagctgccatcagtcggcgtggactgtagaacactgccaatgccggtcccaagcccggataaaagtggagggtacagtccacgcttttttttgaatacattgaaaagctcgccgtaagtcccttctctccctgggtggatggtggagtgctttttaagctaagcagcttggcgtaactagatct

SEQ ID NO: 494 - DNA, 350 bp, unidentified
  source 1..350: mol_type = unassigned DNA
ggccaaagcatgcatgcatccacccagggagagaagtttcagagctatgctggaaacagcatagcaagttgaaataaggctagtccgttatcaacttgaaaaagtggcaccgagtcggtgcattgaaaagctcgccacagtaagtcccttctctccctgggtggaaaaaaaaagctgccatcagtcggcgtggactgtagaacactgccaatgccggtcccaagcccggataaaagtggagggtacagtccacgctttttttacattgaaaagctcgccgtaagtcccttctctccctgggtggatggtggagtgctaaacacgctaagcagcttggcgtaactagatct

SEQ ID NO: 495 - DNA, 350 bp, unidentified
  source 1..350: mol_type = unassigned DNA
ggccaaagcatgcatgcagggagagaagggacttagtttcagagctatgctggaaacagcatagcaagttgaaataaggctagtccgttatcaacttgaaaaagtggcaccgagtcggtgcgccacagtaagtcccttctctccaaaaaaaaagctgccatcagtcggcgtggactgtagaacactgccaatgccggtcccaagcccggataaaagtggagggtacagtccacgctttttttcttgcctctctgggtgcacctgatgaatacattgaaaagctcgccgtaagtcccttctctccctgggtggatggttctggagctaagcagcttggcgtaactagatct

SEQ ID NO: 496 - DNA, 350 bp, unidentified
  source 1..350: mol_type = unassigned DNA
ggccaaagcatgcatgcagggagagaagggacttagtttcagagctatgctggaaacagcatagcaagttgaaataaggctagtccgttatcaacttgaaaaagtggcaccgagtcggtgcgctcgccacagtaagtcccttctctccaaaaaaaaagctgccatcagtcggcgtggactgtagaacactgccaatgccggtcccaagcccggataaaagtggagggtacagtccacgctttttttcctctctgggtgcacctgatgaatacattgaaaagctcgccgtaagtcccttctctccctgggtggatggtacagtagctaagcagcttggcgtaactagatct

SEQ ID NO: 497 - DNA, 350 bp, unidentified
  source 1..350: mol_type = unassigned DNA
ggccaaagcatgcatgcagggagagaagggacttagtttcagagctatgctggaaacagcatagcaagttgaaataaggctagtccgttatcaacttgaaaaagtggcaccgagtcggtgcaaagctcgccacagtaagtcccttctctccaaaaaaaaagctgccatcagtcggcgtggactgtagaacactgccaatgccggtcccaagcccggataaaagtggagggtacagtccacgctttttttctctgggtgcacctgatgaatacattgaaaagctcgccgtaagtcccttctctccctgggtggatggtgggcccgctaagcagcttggcgtaactagatct

SEQ ID NO: 498 - DNA, 350 bp, unidentified
  source 1..350: mol_type = unassigned DNA
ggccaaagcatgcatgcagggagagaagggacttagtttcagagctatgctggaaacagcatagcaagttgaaataaggctagtccgttatcaacttgaaaaagtggcaccgagtcggtgcattgaaaagctcgccacagtaagtcccttctctccaaaaaaaaagctgccatcagtcggcgtggactgtagaacactgccaatgccggtcccaagcccggataaaagtggagggtacagtccacgctttttttggtgcacctgatgaatacattgaaaagctcgccgtaagtcccttctctccctgggtggatggttatttggctaagcagcttggcgtaactagatct

SEQ ID NO: 499 - DNA, 350 bp, unidentified
  source 1..350: mol_type = unassigned DNA
ggccaaagcatgcatgctaacaggtattgagcatggtttcagagctatgctggaaacagcatagcaagttgaaataaggctagtccgttatcaacttgaaaaagtggcaccgagtcggtgcgaggttagtgccacatgctcaatacctgtaaaaaaaaagctgccatcagtcggcgtggactgtagaacactgccaatgccggtcccaagcccggataaaagtggagggtacagtccacgctttttttgctatgaaggcttctttggactgtatagagattagtgccacatgctcaatacctgttaggtgaaataactctattgctaagcagcttggcgtaactagatct

SEQ ID NO: 500 - DNA, 350 bp, unidentified
  source 1..350: mol_type = unassigned DNA
ggccaaagcatgcatgctaacaggtattgagcatggtttcagagctatgctggaaacagcatagcaagttgaaataaggctagtccgttatcaacttgaaaaagtggcaccgagtcggtgctatagaggttagtgccacatgctcaatacctgtaaaaaaaaagctgccatcagtcggcgtggactgtagaacactgccaatgccggtcccaagcccggataaaagtggagggtacagtccacgcttttttttgaaggcttctttggactgtatagagattagtgccacatgctcaatacctgttaggtgaaataacaggcgcgctaagcagcttggcgtaactagatct

SEQ ID NO: 501 - DNA, 350 bp, unidentified
  source 1..350: mol_type = unassigned DNA
ggccaaagcatgcatgctaacaggtattgagcatggtttcagagctatgctggaaacagcatagcaagttgaaataaggctagtccgttatcaacttgaaaaagtggcaccgagtcggtgcctgtatagaggttagtgccacatgctcaatacctgtaaaaaaaaagctgccatcagtcggcgtggactgtagaacactgccaatgccggtcccaagcccggataaaagtggagggtacagtccacgctttttttaggcttctttggactgtatagagattagtgccacatgctcaatacctgttaggtgaaataactgtttggctaagcagcttggcgtaactagatct

SEQ ID NO: 502 - DNA, 350 bp, unidentified
  source 1..350: mol_type = unassigned DNA
ggccaaagcatgcatgctaacaggtattgagcatggtttcagagctatgctggaaacagcatagcaagttgaaataaggctagtccgttatcaacttgaaaaagtggcaccgagtcggtgcttggactgtatagaggttagtgccacatgctcaatacctgtaaaaaaaaagctgccatcagtcggcgtggactgtagaacactgccaatgccggtcccaagcccggataaaagtggagggtacagtccacgcttttttttctttggactgtatagagattagtgccacatgctcaatacctgttaggtgaaataacgcgagggctaagcagcttggcgtaactagatct

SEQ ID NO: 503 - DNA, 350 bp, unidentified
  source 1..350: mol_type = unassigned DNA
ggccaaagcatgcatggagtctcgggtcagtgccagtttcagagctatgctggaaacagcatagcaagttgaaataaggctagtccgttatcaacttgaaaaagtggcaccgagtcggtgccagccacgccgtggcactgacccgagaaaaaaaaaagctgccatcagtcggcgtggactgtagaacactgccaatgccggtcccaagcccggataaaagtggagggtacagtccacgctttttttctgccctgaccctcctgcccctggcttcccacagccacgccatggcactgacccgagactctgagcggctggagtttgctaagcagcttggcgtaactagatct

SEQ ID NO: 504 - DNA, 350 bp, unidentified
  source 1..350: mol_type = unassigned DNA
ggccaaagcatgcatggagtctcgggtcagtgccagtttcagagctatgctggaaacagcatagcaagttgaaataaggctagtccgttatcaacttgaaaaagtggcaccgagtcggtgctcccacagccacgccgtggcactgacccgagaaaaaaaaaagctgccatcagtcggcgtggactgtagaacactgccaatgccggtcccaagcccggataaaagtggagggtacagtccacgctttttttctgaccctcctgcccctggcttcccacagccacgccatggcactgacccgagactctgagcggctgagcagagctaagcagcttggcgtaactagatct

SEQ ID NO: 505 - DNA, 350 bp, unidentified
  source 1..350: mol_type = unassigned DNA
ggccaaagcatgcatgttttaattgttcagccatggtttcagagctatgctggaaacagcatagcaagttgaaataaggctagtccgttatcaacttgaaaaagtggcaccgagtcggtgcctcgtggctgaacaattaaaaaaaaaagctgccatcagtcggcgtggactgtagaacactgccaatgccggtcccaagcccggataaaagtggagggtacagtccacgcttttttttcaagcagaccagcctttctcaatactgtagggccttcttccacccctcctcatggctgaacaattaaaaaataaagtaccggacagctaagcagcttggcgtaactagatct

SEQ ID NO: 506 - DNA, 350 bp, unidentified
  source 1..350: mol_type = unassigned DNA
ggccaaagcatgcatgttttaattgttcagccatggtttcagagctatgctggaaacagcatagcaagttgaaataaggctagtccgttatcaacttgaaaaagtggcaccgagtcggtgccctcctcgtggctgaacaattaaaaaaaaaagctgccatcagtcggcgtggactgtagaacactgccaatgccggtcccaagcccggataaaagtggagggtacagtccacgctttttttgcagaccagcctttctcaatactgtagggccttcttccacccctcctcatggctgaacaattaaaaaataaagtacgtcctggctaagcagcttggcgtaactagatct

SEQ ID NO: 507 - DNA, 350 bp, unidentified
  source 1..350: mol_type = unassigned DNA
ggccaaagcatgcatgttttaattgttcagccatggtttcagagctatgctggaaacagcatagcaagttgaaataaggctagtccgttatcaacttgaaaaagtggcaccgagtcggtgccttccacccctcctcgtggctgaacaattaaaaaaaaaagctgccatcagtcggcgtggactgtagaacactgccaatgccggtcccaagcccggataaaagtggagggtacagtccacgctttttttgcctttctcaatactgtagggccttcttccacccctcctcatggctgaacaattaaaaaataaagtacaaggtggctaagcagcttggcgtaactagatct

SEQ ID NO: 508 - DNA, 350 bp, unidentified
  source 1..350: mol_type = unassigned DNA
ggccaaagcatgcatgcttcttccacccctcctcagtttcagagctatgctggaaacagcatagcaagttgaaataaggctagtccgttatcaacttgaaaaagtggcaccgagtcggtgcccacgaggaggggtggaagaaaaaaaaagctgccatcagtcggcgtggactgtagaacactgccaatgccggtcccaagcccggataaaagtggagggtacagtccacgcttttttttgatggtcatgataatgtctgaagtactttattttttaattgttcagccatgaggaggggtggaagaaggccctacagtgattgcgctaagcagcttggcgtaactagatct

SEQ ID NO: 509 - DNA, 350 bp, unidentified
  source 1..350: mol_type = unassigned DNA
ggccaaagcatgcatgcttcttccacccctcctcagtttcagagctatgctggaaacagcatagcaagttgaaataaggctagtccgttatcaacttgaaaaagtggcaccgagtcggtgctcagccacgaggaggggtggaagaaaaaaaaagctgccatcagtcggcgtggactgtagaacactgccaatgccggtcccaagcccggataaaagtggagggtacagtccacgctttttttggtcatgataatgtctgaagtactttattttttaattgttcagccatgaggaggggtggaagaaggccctacagtgtgctggctaagcagcttggcgtaactagatct

SEQ ID NO: 510 - DNA, 350 bp, unidentified
  source 1..350: mol_type = unassigned DNA
ggccaaagcatgcatgcttcttccacccctcctcagtttcagagctatgctggaaacagcatagcaagttgaaataaggctagtccgttatcaacttgaaaaagtggcaccgagtcggtgctgttcagccacgaggaggggtggaagaaaaaaaaagctgccatcagtcggcgtggactgtagaacactgccaatgccggtcccaagcccggataaaagtggagggtacagtccacgctttttttcatgataatgtctgaagtactttattttttaattgttcagccatgaggaggggtggaagaaggccctacagtccggaagctaagcagcttggcgtaactagatct

SEQ ID NO: 511 - DNA, 350 bp, unidentified
  source 1..350: mol_type = unassigned DNA
ggccaaagcatgcatgcttcttccacccctcctcagtttcagagctatgctggaaacagcatagcaagttgaaataaggctagtccgttatcaacttgaaaaagtggcaccgagtcggtgcttaattgttcagccacgaggaggggtggaagaaaaaaaaagctgccatcagtcggcgtggactgtagaacactgccaatgccggtcccaagcccggataaaagtggagggtacagtccacgcttttttttaatgtctgaagtactttattttttaattgttcagccatgaggaggggtggaagaaggccctacagtggcacggctaagcagcttggcgtaactagatct

SEQ ID NO: 512 - DNA, 350 bp, unidentified
  source 1..350: mol_type = unassigned DNA
ggccaaagcatgcatgagcatggcagatgcagtatgtttcagagctatgctggaaacagcatagcaagttgaaataaggctagtccgttatcaacttgaaaaagtggcaccgagtcggtgcactcgagccaatactgcatctgccataaaaaaaaagctgccatcagtcggcgtggactgtagaacactgccaatgccggtcccaagcccggataaaagtggagggtacagtccacgctttttttttagccagggtgtccaaatctgattgtttatacacttgagccaatactgcatctgccatgctagacaatacacattatgctaagcagcttggcgtaactagatct

SEQ ID NO: 513 - DNA, 350 bp, unidentified
  source 1..350: mol_type = unassigned DNA
ggccaaagcatgcatgagcatggcagatgcagtatgtttcagagctatgctggaaacagcatagcaagttgaaataaggctagtccgttatcaacttgaaaaagtggcaccgagtcggtgcatacactcgagccaatactgcatctgccataaaaaaaaagctgccatcagtcggcgtggactgtagaacactgccaatgccggtcccaagcccggataaaagtggagggtacagtccacgctttttttccagggtgtccaaatctgattgtttatacacttgagccaatactgcatctgccatgctagacaatacacgtcttgctaagcagcttggcgtaactagatct

SEQ ID NO: 514 - DNA, 350 bp, unidentified
  source 1..350: mol_type = unassigned DNA
ggccaaagcatgcatgagcatggcagatgcagtatgtttcagagctatgctggaaacagcatagcaagttgaaataaggctagtccgttatcaacttgaaaaagtggcaccgagtcggtgctttatacactcgagccaatactgcatctgccataaaaaaaaagctgccatcagtcggcgtggactgtagaacactgccaatgccggtcccaagcccggataaaagtggagggtacagtccacgctttttttgggtgtccaaatctgattgtttatacacttgagccaatactgcatctgccatgctagacaatacaacggcagctaagcagcttggcgtaactagatct

SEQ ID NO: 515 - DNA, 350 bp, unidentified
  source 1..350: mol_type = unassigned DNA
ggccaaagcatgcatgagcatggcagatgcagtatgtttcagagctatgctggaaacagcatagcaagttgaaataaggctagtccgttatcaacttgaaaaagtggcaccgagtcggtgcgattgtttatacactcgagccaatactgcatctgccataaaaaaaaagctgccatcagtcggcgtggactgtagaacactgccaatgccggtcccaagcccggataaaagtggagggtacagtccacgcttttttttccaaatctgattgtttatacacttgagccaatactgcatctgccatgctagacaatacacggagtgctaagcagcttggcgtaactagatct

SEQ ID NO: 516 - DNA, 350 bp, unidentified
  source 1..350: mol_type = unassigned DNA
ggccaaagcatgcatgcaggagaatcctggcacatgtttcagagctatgctggaaacagcatagcaagttgaaataaggctagtccgttatcaacttgaaaaagtggcaccgagtcggtgcttagattcccgatgtgccaggattctcaaaaaaaaagctgccatcagtcggcgtggactgtagaacactgccaatgccggtcccaagcccggataaaagtggagggtacagtccacgcttttttttcatacataaccaagtgaaaacattgtaattttacattcccgatgtgccaggattctcctgggtgactccctccatggctaagcagcttggcgtaactagatct

SEQ ID NO: 517 - DNA, 350 bp, unidentified
  source 1..350: mol_type = unassigned DNA
ggccaaagcatgcatgcaggagaatcctggcacatgtttcagagctatgctggaaacagcatagcaagttgaaataaggctagtccgttatcaacttgaaaaagtggcaccgagtcggtgcaattttagattcccgatgtgccaggattctcaaaaaaaaagctgccatcagtcggcgtggactgtagaacactgccaatgccggtcccaagcccggataaaagtggagggtacagtccacgctttttttacataaccaagtgaaaacattgtaattttacattcccgatgtgccaggattctcctgggtgactcccaacttagctaagcagcttggcgtaactagatct

SEQ ID NO: 518 - DNA, 350 bp, unidentified
  source 1..350: mol_type = unassigned DNA
ggccaaagcatgcatgcaggagaatcctggcacatgtttcagagctatgctggaaacagcatagcaagttgaaataaggctagtccgttatcaacttgaaaaagtggcaccgagtcggtgctgtaattttagattcccgatgtgccaggattctcaaaaaaaaagctgccatcagtcggcgtggactgtagaacactgccaatgccggtcccaagcccggataaaagtggagggtacagtccacgcttttttttaaccaagtgaaaacattgtaattttacattcccgatgtgccaggattctcctgggtgactccccaagaggctaagcagcttggcgtaactagatct

SEQ ID NO: 519 - DNA, 350 bp, unidentified
  source 1..350: mol_type = unassigned DNA
ggccaaagcatgcatgcaggagaatcctggcacatgtttcagagctatgctggaaacagcatagcaagttgaaataaggctagtccgttatcaacttgaaaaagtggcaccgagtcggtgcaacattgtaattttagattcccgatgtgccaggattctcaaaaaaaaagctgccatcagtcggcgtggactgtagaacactgccaatgccggtcccaagcccggataaaagtggagggtacagtccacgctttttttaagtgaaaacattgtaattttacattcccgatgtgccaggattctcctgggtgactcccctggccgctaagcagcttggcgtaactagatct

SEQ ID NO: 520 - DNA, 350 bp, unidentified
  source 1..350: mol_type = unassigned DNA
ggccaaagcatgcatgaggagaatcctggcacatcgtttcagagctatgctggaaacagcatagcaagttgaaataaggctagtccgttatcaacttgaaaaagtggcaccgagtcggtgcttagattcccgatgtgccaggattctaaaaaaaaagctgccatcagtcggcgtggactgtagaacactgccaatgccggtcccaagcccggataaaagtggagggtacagtccacgctttttttaatcatacataaccaagtgaaaacattgtaattttacattcccgatgtgccaggattctcctgggtgactcccgaaaagctaagcagcttggcgtaactagatct

SEQ ID NO: 521 - DNA, 350 bp, unidentified
  source 1..350: mol_type = unassigned DNA
ggccaaagcatgcatgaggagaatcctggcacatcgtttcagagctatgctggaaacagcatagcaagttgaaataaggctagtccgttatcaacttgaaaaagtggcaccgagtcggtgcaattttagattcccgatgtgccaggattctaaaaaaaaagctgccatcagtcggcgtggactgtagaacactgccaatgccggtcccaagcccggataaaagtggagggtacagtccacgctttttttatacataaccaagtgaaaacattgtaattttacattcccgatgtgccaggattctcctgggtgactcctagccagctaagcagcttggcgtaactagatct

SEQ ID NO: 522 - DNA, 350 bp, unidentified
  source 1..350: mol_type = unassigned DNA
ggccaaagcatgcatgaggagaatcctggcacatcgtttcagagctatgctggaaacagcatagcaagttgaaataaggctagtccgttatcaacttgaaaaagtggcaccgagtcggtgctgtaattttagattcccgatgtgccaggattctaaaaaaaaagctgccatcagtcggcgtggactgtagaacactgccaatgccggtcccaagcccggataaaagtggagggtacagtccacgctttttttcataaccaagtgaaaacattgtaattttacattcccgatgtgccaggattctcctgggtgactccagaagagctaagcagcttggcgtaactagatct

SEQ ID NO: 523 - DNA, 350 bp, unidentified
  source 1..350: mol_type = unassigned DNA
ggccaaagcatgcatgaggagaatcctggcacatcgtttcagagctatgctggaaacagcatagcaagttgaaataaggctagtccgttatcaacttgaaaaagtggcaccgagtcggtgcaacattgtaattttagattcccgatgtgccaggattctaaaaaaaaagctgccatcagtcggcgtggactgtagaacactgccaatgccggtcccaagcccggataaaagtggagggtacagtccacgctttttttccaagtgaaaacattgtaattttacattcccgatgtgccaggattctcctgggtgactcctacggcgctaagcagcttggcgtaactagatct

SEQ ID NO: 524 - DNA, 350 bp, unidentified
  source 1..350: mol_type = unassigned DNA
ggccaaagcatgcatgcatccatcccaattatcaggtttcagagctatgctggaaacagcatagcaagttgaaataaggctagtccgttatcaacttgaaaaagtggcaccgagtcggtgcccattgataattgggatggaaaaaaaaagctgccatcagtcggcgtggactgtagaacactgccaatgccggtcccaagcccggataaaagtggagggtacagtccacgcttttttttgaggtaatcagccaggatctggataggatggtacaaatctgacagcccactgataattgggatggatgcttctttagcaccggggctaagcagcttggcgtaactagatct

SEQ ID NO: 525 - DNA, 350 bp, unidentified
  source 1..350: mol_type = unassigned DNA
ggccaaagcatgcatgcatccatcccaattatcaggtttcagagctatgctggaaacagcatagcaagttgaaataaggctagtccgttatcaacttgaaaaagtggcaccgagtcggtgccagcccattgataattgggatggaaaaaaaaagctgccatcagtcggcgtggactgtagaacactgccaatgccggtcccaagcccggataaaagtggagggtacagtccacgctttttttgtaatcagccaggatctggataggatggtacaaatctgacagcccactgataattgggatggatgcttctttagcccaaatgctaagcagcttggcgtaactagatct

SEQ ID NO: 526 - DNA, 350 bp, unidentified
  source 1..350: mol_type = unassigned DNA
ggccaaagcatgcatgcatccatcccaattatcaggtttcagagctatgctggaaacagcatagcaagttgaaataaggctagtccgttatcaacttgaaaaagtggcaccgagtcggtgctgacagcccattgataattgggatggaaaaaaaaagctgccatcagtcggcgtggactgtagaacactgccaatgccggtcccaagcccggataaaagtggagggtacagtccacgctttttttatcagccaggatctggataggatggtacaaatctgacagcccactgataattgggatggatgcttctttagctataacgctaagcagcttggcgtaactagatct

SEQ ID NO: 527 - DNA, 350 bp, unidentified
  source 1..350: mol_type = unassigned DNA
ggccaaagcatgcatgcatccatcccaattatcaggtttcagagctatgctggaaacagcatagcaagttgaaataaggctagtccgttatcaacttgaaaaagtggcaccgagtcggtgcaaatctgacagcccattgataattgggatggaaaaaaaaagctgccatcagtcggcgtggactgtagaacactgccaatgccggtcccaagcccggataaaagtggagggtacagtccacgctttttttccaggatctggataggatggtacaaatctgacagcccactgataattgggatggatgcttctttagcgacctagctaagcagcttggcgtaactagatct

SEQ ID NO: 528 - DNA, 350 bp, unidentified
  source 1..350: mol_type = unassigned DNA
ggccaaagcatgcatgatccatcccaattatcagtgtttcagagctatgctggaaacagcatagcaagttgaaataaggctagtccgttatcaacttgaaaaagtggcaccgagtcggtgcccattgataattgggatgaaaaaaaaagctgccatcagtcggcgtggactgtagaacactgccaatgccggtcccaagcccggataaaagtggagggtacagtccacgctttttttcgtgaggtaatcagccaggatctggataggatggtacaaatctgacagcccactgataattgggatggatgcttctttagcaccatgctaagcagcttggcgtaactagatct

SEQ ID NO: 529 - DNA, 350 bp, unidentified
  source 1..350: mol_type = unassigned DNA
ggccaaagcatgcatgatccatcccaattatcagtgtttcagagctatgctggaaacagcatagcaagttgaaataaggctagtccgttatcaacttgaaaaagtggcaccgagtcggtgccagcccattgataattgggatgaaaaaaaaagctgccatcagtcggcgtggactgtagaacactgccaatgccggtcccaagcccggataaaagtggagggtacagtccacgctttttttaggtaatcagccaggatctggataggatggtacaaatctgacagcccactgataattgggatggatgcttctttagcgcgccgctaagcagcttggcgtaactagatct

SEQ ID NO: 530 - DNA, 350 bp, unidentified
  source 1..350: mol_type = unassigned DNA
ggccaaagcatgcatgatccatcccaattatcagtgtttcagagctatgctggaaacagcatagcaagttgaaataaggctagtccgttatcaacttgaaaaagtggcaccgagtcggtgctgacagcccattgataattgggatgaaaaaaaaagctgccatcagtcggcgtggactgtagaacactgccaatgccggtcccaagcccggataaaagtggagggtacagtccacgcttttttttaatcagccaggatctggataggatggtacaaatctgacagcccactgataattgggatggatgcttctttaggtaggcgctaagcagcttggcgtaactagatct

SEQ ID NO: 531 - DNA, 350 bp, unidentified
  source 1..350: mol_type = unassigned DNA
ggccaaagcatgcatgatccatcccaattatcagtgtttcagagctatgctggaaacagcatagcaagttgaaataaggctagtccgttatcaacttgaaaaagtggcaccgagtcggtgcaaatctgacagcccattgataattgggatgaaaaaaaaagctgccatcagtcggcgtggactgtagaacactgccaatgccggtcccaagcccggataaaagtggagggtacagtccacgctttttttagccaggatctggataggatggtacaaatctgacagcccactgataattgggatggatgcttctttagcatatcgctaagcagcttggcgtaactagatct

SEQ ID NO: 532 - DNA, 350 bp, unidentified
  source 1..350: mol_type = unassigned DNA
ggccaaagcatgcatgtcattcattgtgttaaatagtttcagagctatgctggaaacagcatagcaagttgaaataaggctagtccgttatcaacttgaaaaagtggcaccgagtcggtgcttcggtgccatatttaacacaatgaaaaaaaaaaagctgccatcagtcggcgtggactgtagaacactgccaatgccggtcccaagcccggataaaagtggagggtacagtccacgcttttttttctcatcttatgttctgtcaatccctttgatttttcagtgccatatttaacacaatgaatgaatagagaacatgagtagctaagcagcttggcgtaactagatct

SEQ ID NO: 533 - DNA, 350 bp, unidentified
  source 1..350: mol_type = unassigned DNA
ggccaaagcatgcatgtcattcattgtgttaaatagtttcagagctatgctggaaacagcatagcaagttgaaataaggctagtccgttatcaacttgaaaaagtggcaccgagtcggtgcatttttcggtgccatatttaacacaatgaaaaaaaaaaagctgccatcagtcggcgtggactgtagaacactgccaatgccggtcccaagcccggataaaagtggagggtacagtccacgctttttttatcttatgttctgtcaatccctttgatttttcagtgccatatttaacacaatgaatgaatagagaacacgccccgctaagcagcttggcgtaactagatct

SEQ ID NO: 534 - DNA, 350 bp, unidentified
  source 1..350: mol_type = unassigned DNA
ggccaaagcatgcatgtcattcattgtgttaaatagtttcagagctatgctggaaacagcatagcaagttgaaataaggctagtccgttatcaacttgaaaaagtggcaccgagtcggtgcttgatttttcggtgccatatttaacacaatgaaaaaaaaaaagctgccatcagtcggcgtggactgtagaacactgccaatgccggtcccaagcccggataaaagtggagggtacagtccacgctttttttttatgttctgtcaatccctttgatttttcagtgccatatttaacacaatgaatgaatagagaacatctagggctaagcagcttggcgtaactagatct

SEQ ID NO: 535 - DNA, 350 bp, unidentified
  source 1..350: mol_type = unassigned DNA
ggccaaagcatgcatgtcattcattgtgttaaatagtttcagagctatgctggaaacagcatagcaagttgaaataaggctagtccgttatcaacttgaaaaagtggcaccgagtcggtgctccctttgatttttcggtgccatatttaacacaatgaaaaaaaaaaagctgccatcagtcggcgtggactgtagaacactgccaatgccggtcccaagcccggataaaagtggagggtacagtccacgctttttttttctgtcaatccctttgatttttcagtgccatatttaacacaatgaatgaatagagaacatttcaagctaagcagcttggcgtaactagatct

SEQ ID NO: 536 - DNA, 350 bp, unidentified
  source 1..350: mol_type = unassigned DNA
ggccaaagcatgcatgggaggaagaaaagaaaacagtttcagagctatgctggaaacagcatagcaagttgaaataaggctagtccgttatcaacttgaaaaagtggcaccgagtcggtgcaacactgtgccccatgttttcttttcttccaaaaaaaaagctgccatcagtcggcgtggactgtagaacactgccaatgccggtcccaagcccggataaaagtggagggtacagtccacgcttttttttttatacaaggacttcagagtcttgaacgctgtgccccatgttttcttttcttcctccatgtattccaacacgggctaagcagcttggcgtaactagatct

SEQ ID NO: 537 - DNA, 350 bp, unidentified
  source 1..350: mol_type = unassigned DNA
ggccaaagcatgcatgggaggaagaaaagaaaacagtttcagagctatgctggaaacagcatagcaagttgaaataaggctagtccgttatcaacttgaaaaagtggcaccgagtcggtgccttgaacactgtgccccatgttttcttttcttccaaaaaaaaagctgccatcagtcggcgtggactgtagaacactgccaatgccggtcccaagcccggataaaagtggagggtacagtccacgcttttttttacaaggacttcagagtcttgaacgctgtgccccatgttttcttttcttcctccatgtattccacatgaggctaagcagcttggcgtaactagatct

SEQ ID NO: 538 - DNA, 350 bp, unidentified
  source 1..350: mol_type = unassigned DNA
ggccaaagcatgcatgggaggaagaaaagaaaacagtttcagagctatgctggaaacagcatagcaagttgaaataaggctagtccgttatcaacttgaaaaagtggcaccgagtcggtgcagtcttgaacactgtgccccatgttttcttttcttccaaaaaaaaagctgccatcagtcggcgtggactgtagaacactgccaatgccggtcccaagcccggataaaagtggagggtacagtccacgctttttttaaggacttcagagtcttgaacgctgtgccccatgttttcttttcttcctccatgtattccacacaatgctaagcagcttggcgtaactagatct

SEQ ID NO: 539 - DNA, 350 bp, unidentified
  source 1..350: mol_type = unassigned DNA
ggccaaagcatgcatggaggaagaaaagaaaacatgtttcagagctatgctggaaacagcatagcaagttgaaataaggctagtccgttatcaacttgaaaaagtggcaccgagtcggtgcaacactgtgccccatgttttcttttcttcaaaaaaaaagctgccatcagtcggcgtggactgtagaacactgccaatgccggtcccaagcccggataaaagtggagggtacagtccacgctttttttgttttatacaaggacttcagagtcttgaacgctgtgccccatgttttcttttcttcctccatgtattcctgcactgctaagcagcttggcgtaactagatct

SEQ ID NO: 540 - DNA, 350 bp, unidentified
  source 1..350: mol_type = unassigned DNA
ggccaaagcatgcatggaggaagaaaagaaaacatgtttcagagctatgctggaaacagcatagcaagttgaaataaggctagtccgttatcaacttgaaaaagtggcaccgagtcggtgccttgaacactgtgccccatgttttcttttcttcaaaaaaaaagctgccatcagtcggcgtggactgtagaacactgccaatgccggtcccaagcccggataaaagtggagggtacagtccacgcttttttttatacaaggacttcagagtcttgaacgctgtgccccatgttttcttttcttcctccatgtattcctggcatgctaagcagcttggcgtaactagatct

SEQ ID NO: 541 - DNA, 350 bp, unidentified
  source 1..350: mol_type = unassigned DNA
ggccaaagcatgcatggaggaagaaaagaaaacatgtttcagagctatgctggaaacagcatagcaagttgaaataaggctagtccgttatcaacttgaaaaagtggcaccgagtcggtgcagtcttgaacactgtgccccatgttttcttttcttcaaaaaaaaagctgccatcagtcggcgtggactgtagaacactgccaatgccggtcccaagcccggataaaagtggagggtacagtccacgctttttttacaaggacttcagagtcttgaacgctgtgccccatgttttcttttcttcctccatgtattccgttcctgctaagcagcttggcgtaactagatct

SEQ ID NO: 542 - DNA, 350 bp, unidentified
  source 1..350: mol_type = unassigned DNA
ggccaaagcatgcatggaggaagaaaagaaaacatgtttcagagctatgctggaaacagcatagcaagttgaaataaggctagtccgttatcaacttgaaaaagtggcaccgagtcggtgcttcagagtcttgaacactgtgccccatgttttcttttcttcaaaaaaaaagctgccatcagtcggcgtggactgtagaacactgccaatgccggtcccaagcccggataaaagtggagggtacagtccacgctttttttgacttcagagtcttgaacgctgtgccccatgttttcttttcttcctccatgtattccatagctgctaagcagcttggcgtaactagatct

SEQ ID NO: 543 - DNA, 350 bp, unidentified
  source 1..350: mol_type = unassigned DNA
ggccaaagcatgcatgaggaagaaaagaaaacatggtttcagagctatgctggaaacagcatagcaagttgaaataaggctagtccgttatcaacttgaaaaagtggcaccgagtcggtgcaacactgtgccccatgttttcttttcttaaaaaaaaagctgccatcagtcggcgtggactgtagaacactgccaatgccggtcccaagcccggataaaagtggagggtacagtccacgctttttttgggttttatacaaggacttcagagtcttgaacgctgtgccccatgttttcttttcttcctccatgtattcgtatgtgctaagcagcttggcgtaactagatct

SEQ ID NO: 544 - DNA, 350 bp, unidentified
  source 1..350: mol_type = unassigned DNA
ggccaaagcatgcatgaggaagaaaagaaaacatggtttcagagctatgctggaaacagcatagcaagttgaaataaggctagtccgttatcaacttgaaaaagtggcaccgagtcggtgcagtcttgaacactgtgccccatgttttcttttcttaaaaaaaaagctgccatcagtcggcgtggactgtagaacactgccaatgccggtcccaagcccggataaaagtggagggtacagtccacgctttttttatacaaggacttcagagtcttgaacgctgtgccccatgttttcttttcttcctccatgtattccctaaggctaagcagcttggcgtaactagatct

SEQ ID NO: 545 - DNA, 350 bp, unidentified
  source 1..350: mol_type = unassigned DNA
ggccaaagcatgcatgaggaagaaaagaaaacatggtttcagagctatgctggaaacagcatagcaagttgaaataaggctagtccgttatcaacttgaaaaagtggcaccgagtcggtgcttcagagtcttgaacactgtgccccatgttttcttttcttaaaaaaaaagctgccatcagtcggcgtggactgtagaacactgccaatgccggtcccaagcccggataaaagtggagggtacagtccacgctttttttaggacttcagagtcttgaacgctgtgccccatgttttcttttcttcctccatgtattctttccagctaagcagcttggcgtaactagatct

SEQ ID NO: 546 - DNA, 350 bp, unidentified
  source 1..350: mol_type = unassigned DNA
ggccaaagcatgcatgaggcagctactccaaagttgtttcagagctatgctggaaacagcatagcaagttgaaataaggctagtccgttatcaacttgaaaaagtggcaccgagtcggtgcctacctttggagtagctgaaaaaaaaagctgccatcagtcggcgtggactgtagaacactgccaatgccggtcccaagcccggataaaagtggagggtacagtccacgctttttttatctgaggatgatttaagagaggggttagtttcaaggcaaaaagtttccctaactttggagtagctgcctgaaggtgcatggtaatgctaagcagcttggcgtaactagatct

SEQ ID NO: 547 - DNA, 350 bp, unidentified
  source 1..350: mol_type = unassigned DNA
ggccaaagcatgcatgaggcagctactccaaagttgtttcagagctatgctggaaacagcatagcaagttgaaataaggctagtccgttatcaacttgaaaaagtggcaccgagtcggtgcttccctacctttggagtagctgaaaaaaaaagctgccatcagtcggcgtggactgtagaacactgccaatgccggtcccaagcccggataaaagtggagggtacagtccacgctttttttgaggatgatttaagagaggggttagtttcaaggcaaaaagtttccctaactttggagtagctgcctgaaggtgcatatcactgctaagcagcttggcgtaactagatct

SEQ ID NO: 548 - DNA, 350 bp, unidentified
  source 1..350: mol_type = unassigned DNA
ggccaaagcatgcatgaggcagctactccaaagttgtttcagagctatgctggaaacagcatagcaagttgaaataaggctagtccgttatcaacttgaaaaagtggcaccgagtcggtgcagtttccctacctttggagtagctgaaaaaaaaagctgccatcagtcggcgtggactgtagaacactgccaatgccggtcccaagcccggataaaagtggagggtacagtccacgctttttttgatgatttaagagaggggttagtttcaaggcaaaaagtttccctaactttggagtagctgcctgaaggtgcattctcgcgctaagcagcttggcgtaactagatct

SEQ ID NO: 549 - DNA, 350 bp, unidentified
  source 1..350: mol_type = unassigned DNA
ggccaaagcatgcatgaggcagctactccaaagttgtttcagagctatgctggaaacagcatagcaagttgaaataaggctagtccgttatcaacttgaaaaagtggcaccgagtcggtgccaaaaagtttccctacctttggagtagctgaaaaaaaaagctgccatcagtcggcgtggactgtagaacactgccaatgccggtcccaagcccggataaaagtggagggtacagtccacgcttttttttttaagagaggggttagtttcaaggcaaaaagtttccctaactttggagtagctgcctgaaggtgcattgaccggctaagcagcttggcgtaactagatct

SEQ ID NO: 550 - DNA, 350 bp, unidentified
  source 1..350: mol_type = unassigned DNA
ggccaaagcatgcatgggcagctactccaaagttagtttcagagctatgctggaaacagcatagcaagttgaaataaggctagtccgttatcaacttgaaaaagtggcaccgagtcggtgcctacctttggagtagctaaaaaaaaagctgccatcagtcggcgtggactgtagaacactgccaatgccggtcccaagcccggataaaagtggagggtacagtccacgctttttttgcatctgaggatgatttaagagaggggttagtttcaaggcaaaaagtttccctaactttggagtagctgcctgaaggtgcatatgttgctaagcagcttggcgtaactagatct

SEQ ID NO: 551 - DNA, 350 bp, unidentified
  source 1..350: mol_type = unassigned DNA
ggccaaagcatgcatgggcagctactccaaagttagtttcagagctatgctggaaacagcatagcaagttgaaataaggctagtccgttatcaacttgaaaaagtggcaccgagtcggtgcttccctacctttggagtagctaaaaaaaaagctgccatcagtcggcgtggactgtagaacactgccaatgccggtcccaagcccggataaaagtggagggtacagtccacgctttttttctgaggatgatttaagagaggggttagtttcaaggcaaaaagtttccctaactttggagtagctgcctgaaggtgcaacaaccgctaagcagcttggcgtaactagatct

SEQ ID NO: 552 - DNA, 350 bp, unidentified
  source 1..350: mol_type = unassigned DNA
ggccaaagcatgcatgggcagctactccaaagttagtttcagagctatgctggaaacagcatagcaagttgaaataaggctagtccgttatcaacttgaaaaagtggcaccgagtcggtgcagtttccctacctttggagtagctaaaaaaaaagctgccatcagtcggcgtggactgtagaacactgccaatgccggtcccaagcccggataaaagtggagggtacagtccacgctttttttaggatgatttaagagaggggttagtttcaaggcaaaaagtttccctaactttggagtagctgcctgaaggtgcagtcgcagctaagcagcttggcgtaactagatct

SEQ ID NO: 553 - DNA, 350 bp, unidentified
  source 1..350: mol_type = unassigned DNA
ggccaaagcatgcatgggcagctactccaaagttagtttcagagctatgctggaaacagcatagcaagttgaaataaggctagtccgttatcaacttgaaaaagtggcaccgagtcggtgccaaaaagtttccctacctttggagtagctaaaaaaaaagctgccatcagtcggcgtggactgtagaacactgccaatgccggtcccaagcccggataaaagtggagggtacagtccacgctttttttgatttaagagaggggttagtttcaaggcaaaaagtttccctaactttggagtagctgcctgaaggtgcacagtcggctaagcagcttggcgtaactagatct

SEQ ID NO: 554 - DNA, 350 bp, unidentified
  source 1..350: mol_type = unassigned DNA
ggccaaagcatgcatgagctggaggacagtactcagtttcagagctatgctggaaacagcatagcaagttgaaataaggctagtccgttatcaacttgaaaaagtggcaccgagtcggtgcatacccccgaaccgtgagtactgtcctccaaaaaaaaaagctgccatcagtcggcgtggactgtagaacactgccaatgccggtcccaagcccggataaaagtggagggtacagtccacgctttttttatcagacatggatccaagcccatgtatatccccgaaccgtgagtactgtcctccagctaccagttgccggccgagctaagcagcttggcgtaactagatct

SEQ ID NO: 555 - DNA, 350 bp, unidentified
  source 1..350: mol_type = unassigned DNA
ggccaaagcatgcatgagctggaggacagtactcagtttcagagctatgctggaaacagcatagcaagttgaaataaggctagtccgttatcaacttgaaaaagtggcaccgagtcggtgcatgtatacccccgaaccgtgagtactgtcctccaaaaaaaaaagctgccatcagtcggcgtggactgtagaacactgccaatgccggtcccaagcccggataaaagtggagggtacagtccacgctttttttgacatggatccaagcccatgtatatccccgaaccgtgagtactgtcctccagctaccagttgcccgtgcagctaagcagcttggcgtaactagatct

SEQ ID NO: 556 - DNA, 350 bp, unidentified
  source 1..350: mol_type = unassigned DNA
ggccaaagcatgcatgagctggaggacagtactcagtttcagagctatgctggaaacagcatagcaagttgaaataaggctagtccgttatcaacttgaaaaagtggcaccgagtcggtgcccaagcccatgtatacccccgaaccgtgagtactgtcctccaaaaaaaaaagctgccatcagtcggcgtggactgtagaacactgccaatgccggtcccaagcccggataaaagtggagggtacagtccacgcttttttttccaagcccatgtatatccccgaaccgtgagtactgtcctccagctaccagttgccactttggctaagcagcttggcgtaactagatct

SEQ ID NO: 557 - DNA, 350 bp, unidentified
  source 1..350: mol_type = unassigned DNA
ggccaaagcatgcatgaggacagtactcacggttcgtttcagagctatgctggaaacagcatagcaagttgaaataaggctagtccgttatcaacttgaaaaagtggcaccgagtcggtgcatacccccgaaccgtgagtactgtaaaaaaaaagctgccatcagtcggcgtggactgtagaacactgccaatgccggtcccaagcccggataaaagtggagggtacagtccacgcttttttttgcacacagtacatcagacatggatccaagcccatgtatatccccgaaccgtgagtactgtcctccagctaccagccaaagctaagcagcttggcgtaactagatct

SEQ ID NO: 558 - DNA, 350 bp, unidentified
  source 1..350: mol_type = unassigned DNA
ggccaaagcatgcatgaggacagtactcacggttcgtttcagagctatgctggaaacagcatagcaagttgaaataaggctagtccgttatcaacttgaaaaagtggcaccgagtcggtgcatgtatacccccgaaccgtgagtactgtaaaaaaaaagctgccatcagtcggcgtggactgtagaacactgccaatgccggtcccaagcccggataaaagtggagggtacagtccacgctttttttcacagtacatcagacatggatccaagcccatgtatatccccgaaccgtgagtactgtcctccagctaccacaacctgctaagcagcttggcgtaactagatct

SEQ ID NO: 559 - DNA, 350 bp, unidentified
  source 1..350: mol_type = unassigned DNA
ggccaaagcatgcatgaggacagtactcacggttcgtttcagagctatgctggaaacagcatagcaagttgaaataaggctagtccgttatcaacttgaaaaagtggcaccgagtcggtgccccatgtatacccccgaaccgtgagtactgtaaaaaaaaagctgccatcagtcggcgtggactgtagaacactgccaatgccggtcccaagcccggataaaagtggagggtacagtccacgctttttttagtacatcagacatggatccaagcccatgtatatccccgaaccgtgagtactgtcctccagctaccaaacggggctaagcagcttggcgtaactagatct

SEQ ID NO: 560 - DNA, 350 bp, unidentified
  source 1..350: mol_type = unassigned DNA
ggccaaagcatgcatgaggacagtactcacggttcgtttcagagctatgctggaaacagcatagcaagttgaaataaggctagtccgttatcaacttgaaaaagtggcaccgagtcggtgcccaagcccatgtatacccccgaaccgtgagtactgtaaaaaaaaagctgccatcagtcggcgtggactgtagaacactgccaatgccggtcccaagcccggataaaagtggagggtacagtccacgctttttttatcagacatggatccaagcccatgtatatccccgaaccgtgagtactgtcctccagctaccagccccagctaagcagcttggcgtaactagatct

SEQ ID NO: 561 - DNA, 350 bp, unidentified
  source 1..350: mol_type = unassigned DNA
ggccaaagcatgcatgggacagtactcacggttcggtttcagagctatgctggaaacagcatagcaagttgaaataaggctagtccgttatcaacttgaaaaagtggcaccgagtcggtgcatacccccgaaccgtgagtactgaaaaaaaaagctgccatcagtcggcgtggactgtagaacactgccaatgccggtcccaagcccggataaaagtggagggtacagtccacgctttttttactgcacacagtacatcagacatggatccaagcccatgtatatccccgaaccgtgagtactgtcctccagctaccagtcttgctaagcagcttggcgtaactagatct

SEQ ID NO: 562 - DNA, 350 bp, unidentified
  source 1..350: mol_type = unassigned DNA
ggccaaagcatgcatgggacagtactcacggttcggtttcagagctatgctggaaacagcatagcaagttgaaataaggctagtccgttatcaacttgaaaaagtggcaccgagtcggtgccccatgtatacccccgaaccgtgagtactgaaaaaaaaagctgccatcagtcggcgtggactgtagaacactgccaatgccggtcccaagcccggataaaagtggagggtacagtccacgctttttttacagtacatcagacatggatccaagcccatgtatatccccgaaccgtgagtactgtcctccagctacctcaacggctaagcagcttggcgtaactagatct

SEQ ID NO: 563 - DNA, 350 bp, unidentified
  source 1..350: mol_type = unassigned DNA
ggccaaagcatgcatgggacagtactcacggttcggtttcagagctatgctggaaacagcatagcaagttgaaataaggctagtccgttatcaacttgaaaaagtggcaccgagtcggtgcccaagcccatgtatacccccgaaccgtgagtactgaaaaaaaaagctgccatcagtcggcgtggactgtagaacactgccaatgccggtcccaagcccggataaaagtggagggtacagtccacgctttttttacatcagacatggatccaagcccatgtatatccccgaaccgtgagtactgtcctccagctaccccccacgctaagcagcttggcgtaactagatct

SEQ ID NO: 564 - DNA, 350 bp, unidentified
  source 1..350: mol_type = unassigned DNA
ggccaaagcatgcatgcacggttcgggggtatacagtttcagagctatgctggaaacagcatagcaagttgaaataaggctagtccgttatcaacttgaaaaagtggcaccgagtcggtgcaagcccatgtatacccccgaaccaaaaaaaaagctgccatcagtcggcgtggactgtagaacactgccaatgccggtcccaagcccggataaaagtggagggtacagtccacgctttttttcgagtcttccactgcacacagtacatcagacatggatccaagtccatgtatacccccgaaccgtgagtactgtccctctcagctaagcagcttggcgtaactagatct

SEQ ID NO: 565 - DNA, 350 bp, unidentified
  source 1..350: mol_type = unassigned DNA
ggccaaagcatgcatgcacggttcgggggtatacagtttcagagctatgctggaaacagcatagcaagttgaaataaggctagtccgttatcaacttgaaaaagtggcaccgagtcggtgcatccaagcccatgtatacccccgaaccaaaaaaaaagctgccatcagtcggcgtggactgtagaacactgccaatgccggtcccaagcccggataaaagtggagggtacagtccacgcttttttttcttccactgcacacagtacatcagacatggatccaagtccatgtatacccccgaaccgtgagtactgtccatatacgctaagcagcttggcgtaactagatct

SEQ ID NO: 566 - DNA, 350 bp, unidentified
  source 1..350: mol_type = unassigned DNA
ggccaaagcatgcatgcacggttcgggggtatacagtttcagagctatgctggaaacagcatagcaagttgaaataaggctagtccgttatcaacttgaaaaagtggcaccgagtcggtgctggatccaagcccatgtatacccccgaaccaaaaaaaaagctgccatcagtcggcgtggactgtagaacactgccaatgccggtcccaagcccggataaaagtggagggtacagtccacgcttttttttccactgcacacagtacatcagacatggatccaagtccatgtatacccccgaaccgtgagtactgtccgtcttcgctaagcagcttggcgtaactagatct

SEQ ID NO: 567 - DNA, 350 bp, unidentified
  source 1..350: mol_type = unassigned DNA
ggccaaagcatgcatgcacggttcgggggtatacagtttcagagctatgctggaaacagcatagcaagttgaaataaggctagtccgttatcaacttgaaaaagtggcaccgagtcggtgcagacatggatccaagcccatgtatacccccgaaccaaaaaaaaagctgccatcagtcggcgtggactgtagaacactgccaatgccggtcccaagcccggataaaagtggagggtacagtccacgcttttttttgcacacagtacatcagacatggatccaagtccatgtatacccccgaaccgtgagtactgtccgtacgtgctaagcagcttggcgtaactagatct

SEQ ID NO: 568 - DNA, 7924 bp, unidentified
  source 1..7924: mol_type = unassigned DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccatcagtcgccggtcccaagcccggataaaatgggagggggcgggaaaccgcctaaccatgccgactgatggcagaaaaaaaaaaatgcatcgaaggactggaggtgaaggttgaccagagccagtagatcagtgagtcgaaaggcaggctaagcacgcgtgtactagtgtctcgagcttattccagatgcgtgcggatggaattcgagctcggtaccatgccaaaagcaaagcgctatcgcgccttacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgcttttcatgtgtttagtcgaggttaaaaaacgtctaggccccccgaaccacggggacgtggttttcctttgaaaaacacgatgataataacatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggagggcctcctggcgccgcaccggcccaaggagcccgcgtggttcctggccaccgtcggcgtctcgcccgaccaccagggcaagggtctgggcagcgccgtcgtgctccccggagtggaggccgccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctaggctagcttgactgactgagtcgacaatcaaccttttggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaacgcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcgaatgaaagaccccacctgtaggtttggcaagatagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtcgccctgatcattgtcgatcctaccatccactcgacacacccgccagggcccgcatccaccatcgcagacttatcatggatcc

SEQ ID NO: 569-570 - skipped (2 entries)

SEQ ID NO: 571 - RNA, 76 nt, unidentified
  source 1..76: mol_type = unassigned RNA
gttttagagctagaaatagcaagttaaaataaggctagtccgttatcaacttgaaaaagtggcaccgagtcggtgc

SEQ ID NO: 572 - DNA, 76 bp, unidentified
  source 1..76: mol_type = unassigned DNA
gttttagagctagaaatagcaagttaaaataaggctagtccgttatcaacttgaaaaagtgggaccgagtcggtcc

SEQ ID NO: 573 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccatctcgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 574 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccatcgcgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 575 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccatcccgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 576 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccattacgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 577 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccatgacgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 578 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccataacgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 579 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccagcacgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 580 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccaccacgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 581 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccaacacgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 582 - DNA, 492 bp, synthetic construct
  source 1..492: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaacggcagcatagcaagttaaaataaggctagtccgttatcaacttgaaaaagtggcaccgagtcggtgctctgccttcacgtgctcagtctgaaaaaaaaaaaaaaaaaaaaggcccagactgagcacgtgagttttagagctatgctgccgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 583 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccatctcgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 584 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccatcgcgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 585 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccatcccgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 586 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccattacgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 587 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccatgacgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 588 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccataacgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 589 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccagcacgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 590 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccaccacgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 591 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccaacacgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 592 - DNA, 460 bp, synthetic construct
  source 1..460: mol_type = other DNA
gagggcctatttcccatgattccttcatatttgcatatacgatagcttaccgtaacttgaaagtatttcgatttcttggctttatatatcttgtggaaaggacgaaacaccgaaacaccgccatcagtcgccggtcccaagcccggataaaatgggagggggcgggaaaccgcctaaccatgccgactgatggcagaaaaaaaaaaggcccagactgagcacgtgagttttagagctagaaatagcaagttaaaataaggctagtccgttatcaacttgaaaaagtgggaccgagtcggtcctctgccttcacgtgctcagtctgaaaaaaaaagctgccatcagtcggcgtggactgtagaacactgccaatgccggtcccaagcccggataaaagtggagggtacagtccacgcttttttaaaggacaggattaggccaggaaggaggaagcaaggaa

SEQ ID NO: 593 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccatctcgtgctcagtctgttttttaaaggacaggattaggccaggaaggaggaagcaaggaa

SEQ ID NO: 594 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccatcgcgtgctcagtctgttttttaaaggacaggattaggccaggaaggaggaagcaaggaa

SEQ ID NO: 595 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccatcccgtgctcagtctgttttttaaaggacaggattaggccaggaaggaggaagcaaggaa

SEQ ID NO: 596 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccattacgtgctcagtctgttttttaaaggacaggattaggccaggaaggaggaagcaaggaa

SEQ ID NO: 597 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccatgacgtgctcagtctgttttttaaaggacaggattaggccaggaaggaggaagcaaggaa

SEQ ID NO: 598 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccataacgtgctcagtctgttttttaaaggacaggattaggccaggaaggaggaagcaaggaa

SEQ ID NO: 599 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccagcacgtgctcagtctgttttttaaaggacaggattaggccaggaaggaggaagcaaggaa

SEQ ID NO: 600 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccaccacgtgctcagtctgttttttaaaggacaggattaggccaggaaggaggaagcaaggaa

SEQ ID NO: 601 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccaacacgtgctcagtctgttttttaaaggacaggattaggccaggaaggaggaagcaaggaa

SEQ ID NO: 602 - DNA, 163 bp, synthetic construct
  source 1..163: mol_type = other DNA
ggcccagactgagcacgtgagttttagagctagaaatagcaagttaaaataaggctagtccgttatcaacttgaaaaagtgggaccgagtcggtcctctgccttcacgtgctcagtctgttttttaaaggacaggattaggccaggaaggaggaagcaaggaa

SEQ ID NO: 603 - DNA, 140 bp, synthetic construct
  source 1..140: mol_type = other DNA
gtcaaccagtatcccggtgcgttttagagctagaaatagcaagttaaaataaggctagtccgttatcaacttgaaaaagtggcaccgagtcggtgcttttttaaaggacaggattaggccaggaaggaggaagcaaggaa

SEQ ID NO: 604 - DNA, 86 bp, synthetic construct
  source 1..86: mol_type = other DNA
gtttcagagctatgctggaaacagcatagcaagttgaaataaggctagtccgttatcaacttgaaaaagtggcaccgagtcggtgc

SEQ ID NO: 605 - RNA, 15 nt, synthetic construct
  source 1..15: mol_type = other RNA
cggcatcagttcggc

SEQ ID NO: 606 - protein, 8 aa, synthetic construct
VSFTATDL

SEQ ID NO: 607 - protein, 32 aa, synthetic construct
VSSTERYYRSTCFRCLHFRKIFWHCDVMILSL

SEQ ID NO: 608 - DNA, 40 bp, synthetic construct
  source 1..40: mol_type = other DNA
tggggcccagactgagcacgtgatggcagaggaaaggaag

SEQ ID NO: 609 - DNA, 40 bp, synthetic construct; identical to SEQ ID NO: 608
  source 1..40: mol_type = other DNA

SEQ ID NO: 610 - DNA, 40 bp, synthetic construct
  source 1..40: mol_type = other DNA
tggggcccagactgagcacgtgatggcaaaggaaaggaag

SEQ ID NO: 611 - DNA, 40 bp, synthetic construct
  source 1..40: mol_type = other DNA
tggggcccagactgagcacgtgatggcagaggaaaggaac

SEQ ID NO: 612 - DNA, 40 bp, synthetic construct
  source 1..40: mol_type = other DNA
tggggcccagactgagcacgtgatggcaaaggaaaggaac

(19) United States
(12) Patent Application Publication
(10) Pub. No.: US 2025/0313863 A1
(43) Pub. Date: Oct. 9, 2025

(54) ENGINEERED CIRCULARIZED pegRNAs AND USES THEREOF

(71) Applicant: Trustees of Boston University, Boston, MA (US)

(72) Inventors: Milad Babaei Amameh, Needham, MA (US); Alexander Arthur Green, Chestnut Hill, MA (US)

(73) Assignee: Trustees of Boston University, Boston, MA (US)

(21) Appl. No.: 19/174,171

(22) Filed: Apr. 9, 2025

Related U.S. Application Data
(60) Provisional application No. 63/631,858, filed on Apr. 9, 2024.

(51) Int. Cl. C12N 15/90; C12N 9/22; C12N 15/11

(52) U.S. Cl. CPC C12N 15/907; C12N 9/226; C12N 15/11; C12N 2310/20; C12N 2310/351; C12N 2310/532

(57) ABSTRACT

The technology described herein relates to circularized prime editing guide RNAs (cpegRNAs) comprising at least a spacer, a gRNA scaffold, a primer binding site, and a template sequence with one or more nucleotide changes relative to a target sequence. The disclosure also provides compositions and prime editing systems comprising the pegRNAs and uses thereof for prime editing.

### CROSS-REFERENCE TO RELATED APPLICATIONS

This application claims benefit under 35 U.S.C. § 119(e) of U.S. Provisional Application 63/631,858 filed on Apr. 9, 2024, the content of which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 21, 2025, is named 701586-000133USPT_SL.xml and is 729,304 bytes in size.

### TECHNICAL FIELD

The technology described herein relates to circularized prime editing guide RNAs (cpegRNAs), compositions and prime editing systems comprising same and uses thereof.

### BACKGROUND

Genomic modification technologies have a wide range of therapeutic and agricultural applications. Prime editors (PEs) are modular molecular machines that can engineer directed base substitution, deletion, or insertion modifications within the genome of the mammalian and plant cells. Prime editor guide RNAs (pegRNAs) direct the PE molecular machinery for the synthesis of the desired genomic alteration. The design and stability of pegRNAs play essential roles in defining PE performance and require multiple rounds of iterative screening. Nevertheless, PEs function less efficiently in Mismatch Repair (MMR) competent cells that account for most of the therapeutically relevant target cell types. Thus, there remains a need in the art for compositions and methods capable of efficient prime editing in MMR competent cells. The present disclosure addresses this need.

### SUMMARY

Prime editing is a cutting-edge genetic engineering technique designed to correct a wide range of human mutations. Traditional prime editing systems utilize linear prime editing guide RNA (pegRNA), which often degrades quickly within cells, leading to low efficiency, especially in therapeutic applications involving mismatch repair (MMR) competent cells. To address these challenges, a system using involving circularized pegRNA (cpegRNA) is disclosed herein. The circularized form of pegRNA is more stable within cells, evades immune recognition due to the absence of 5′ and 3′ ends, and overcomes safety concerns associated with the requirement for nicking gRNA. Without wishing to be bound by a theory, cpegRNA significantly enhances the functionality of prime editing proteins, with notable increases in editing efficiency in various cell types.

Described herein are advancements in prime editing technology through the development of a circularized pegRNA (cpegRNA) system. Prime editing has faced challenges in efficiency and stability, particularly in mismatch repair (MMR) competent cells. Traditional linear pegRNAsystems degrade quickly within cells, leading to low efficiency in therapeutic applications. Without wishing to be bound by a theory, pegRNAs, e.g., cpegRNAs and prime editing systems described herein offer a solution by enhancing stability and efficiency, evading immune recognition due to the absence of 5′ and 3′ ends, and addressing safety concerns associated with nicking gRNA requirements.

Without wishing to be bound by a theory, the pegRNAs and prime editing systems described herein are compatible with multiple prime editing strategies, including the split PE strategy, which eliminates the need for MCP/MS2 tethering components and allows for packaging in AAV payloads. Additionally, in an aspect of any of the embodiments described herein, is a rotated-pegRNA system to further protect the spacer and primer binding site (PBS) from exonuclease degradation, enhancing stability and editing efficiency. The use of DNA polymerases in the synthesis of circularized rotated pegRNA (cropegRNA) provides additional stability. In an aspect of any of the embodiments described herein, the stability of cpegRNAs show utility for enveloped virus-like particles (eVLP), where the stability of pegRNA is crucial for functionality. Overall, the advancements in cpegRNA technology disclosed herein represent a significant step forward in improving the efficiency and stability of prime editing for therapeutic applications.

In one aspect, provided herein is a prime editing guide RNA(pegRNA). comprising: (a) a spacer domain comprising a sequence substantially complementary to a region of a first strand of a double-stranded target nucleic acid; (b) a gRNA core domain capable of associating with a nucleic acid programmable DNA binding protein (napDNAbp); (c) a nucleic acid synthesis template domain comprising an edit template domain comprising a sequence having one or more nucleotide changes compared to a second strand of the double-stranded target nucleic acid, and optionally the nucleic acid synthesis template domain further comprises a homology arm domain comprising a sequence substantially complementary the second strand of the double-stranded target nucleic acid; and (d) a primer binding site (PBS) comprising a sequence substantially complementary to a region upstream of the region complementary to the nucleic acid synthesis template domain in the second strand of the double-stranded target nucleic acid, and wherein (i) the pegRNA is circularized, or (ii) the pegRNA comprises a first portion of the gRNA core domain at one of the 5′-end or the 3′-end, and a second portion of the gRNA core domain at the other of the 5′-end or the 3′-end, and wherein the first and second portions together form the gRNA core domain; or (iii) the pegRNA comprises a first ribozyme and a first ligation sequence positioned 3′ to the first ribozyme at 5′-end, and a second ribozyme and a second ligation sequence positioned 3′ to the second ribozyme at the 3′-end, and wherein a portion of the first ligation sequence is complementary to a portion of the first ribozyme and a portion of the second ligation sequence is complementary to a portion of the second ribozyme, wherein a portion of the first ligation sequence is complementary to a portion of the second ligation sequence; and wherein the portion of the first ligation sequence complementary to the portion of the first ribozyme is complementary to the portion of the second ligation sequence complementary to the portion of the second ribozyme.

In another aspect, provided herein is a prime editing system comprising: (a) a pegRNA described herein or a nucleic acid encoding same; (b) a nucleic acid programmable DNA binding protein (napDNAbp); and (c) a nucleic acid modifying enzyme.

In yet another aspect, provided herein is a composition comprising a pegRNA described herein or a nucleic acid encoding same. In some embodiments, the composition further comprises napDNAbp or a nucleic acid encoding same; and/or a nucleic acid modifying enzyme or a nucleic acid encoding same.

In another aspect provided herein is a genome-editing composition comprising a cell modified using a method described herein. In some embodiments, the genome-editing composition is selected from the group consisting of: (a) an autologous, ex vivo CRISPR/Cas9 gene-edited hematopoietic stem cell therapy for the treatment of sickle cell disease or β-thalassemia; (b) an allogeneic CRISPR/Cas9 gene-edited CAR T cell therapy targeting CD19+ malignancies and autoimmune diseases; (c) an allogeneic CRISPR/Cas9 gene-edited CAR T cell therapy targeting CD70 for the treatment of solid tumors and hematological malignancies; (d) an in vivo gene-editing therapy utilizing lipid nanoparticle (LNP) delivery to target ANGPTL3 for cardiovascular disease; (e) an in vivo gene-editing therapy utilizing LNP delivery to target Lp(a) for cardiovascular disease; (f) an in vivo gene-editing therapy utilizing LNP delivery to target hepatic angiotensinogen (AGT) for refractory hypertension; (g) an in vivo gene-editing therapy utilizing LNP delivery to target ALAS1 for acute hepatic porphyria (AHP); (h) an allogeneic, gene-edited, immune-evasive, stem cell-derived beta-cell replacement therapy for Type 1 diabetes mellitus; (i) an ex vivo base editing therapy for sickle cell disease to induce fetal hemoglobin expression; (j) a multiplex base edited anti-CD7 CAR-T cell therapy for the treatment of relapsed and refractory T-cell acute lymphoblastic leukemia and T-cell lymphoblastic lymphoma; (k) a liver-targeting LNP-formulated base editing therapy for glycogen storage disease type 1a; (l) a liver-targeting LNP-formulated base editing therapy for Alpha-1 antitrypsin deficiency; (m) an ex vivo autologous hematopoietic stem cell therapy for the treatment of p47\hat{ }phox Chronic Granulomatous Disease; (n) an ex vivo hematopoietic stem cell therapy utilizing a prime editing approach for X-linked Chronic Granulomatous Disease; (o) an in vivo prime editing therapy targeting ATP7B for the treatment of Wilson's Disease; and (p) an in vivo prime editing therapy targeting mutations associated with cystic fibrosis.

In still another aspect provided herein is a kit comprising a pegRNA described herein or a nucleic acid encoding same. In some embodiments, the kit further comprises napDNAbp or a nucleic acid encoding same; and/or a nucleic acid modifying enzyme or a nucleic acid encoding same.

In still yet another provided herein is a cell comprising a pegRNA described herein or a nucleic acid encoding same. In some embodiments, the cell further comprises napDNAbp or a nucleic acid encoding same; and/or a nucleic acid modifying enzyme or a nucleic acid encoding same. In some embodiments, the cell is a mammalian cell. In some embodiments, the cell is a human cell. In some embodiments, the cell is a mismatch repair (MMR) deficient cell. In some embodiments, the cell is a mismatch repair (MMR) competent cell. In some embodiments, the cell is selected from the group consisting of hematopoietic stem cells, T cells, liver cells (e.g., hepatocytes, pancreatic islet beta cells, and lung epithelial cells. In some embodiments, the cell is in vitro. In some embodiments, the cell is ex vivo. In some embodiments, the cell is in vivo. In some embodiments, the cell is a modified cell. In some embodiments, the target nucleic acid is in a cell. In some embodiments, the cell is human cell.

In another aspect, provided herein is a method of introducing one or more changes in the nucleotide sequence of a target nucleic acid, the method comprises: contacting a double-stranded target nucleic acid (e.g., DNA) with a prime editing system described herein. In some embodiments, the target nucleic acid is in a cell. Thus, in some embodiments, the method comprises: contacting or administering to the cell comprising the target nucleic acid: (a) a pegRNA described herein or a nucleic acid encoding same; (b) a nucleic acid programmable DNA binding protein (napDNAbp) or a nucleic acid encoding same; and (c) a nucleic acid modifying enzyme or a nucleic acid encoding same. In some embodiments, the method is a therapeutic gene editing method. For example, the method comprises administering to a target cell selected from the group consisting of: (a) hematopoietic stem cells; (b) T cells; (c) liver cells (hepatocytes); (d) pancreatic islet beta cells; (e) lung epithelial cells.

In some embodiments of any one of the aspects described herein, the spacer domain is 5′ of the gRNA core domain, the gRNA core domain is 5′ of the nucleic acid synthesis template domain, and the nucleic acid synthesis template domain is 5′ the primer binding site. In some embodiments of any one of the aspects described herein, a first portion of the gRNA core domain is 5′ of the nucleic acid synthesis template domain, the nucleic acid synthesis template domain is 5′ of the primer binding site, the primer binding site is 5′ of the spacer domain, and the spacer domain is 5′ of a second portion of the gRNA core domain, and wherein the first and second portions together form the gRNA core domain. In some embodiments of any one of the aspects described herein, a first ligation sequence is 5′ of a portion of the gRNA core domain, the first portion of the gRNA core domain is 5′ of the nucleic acid synthesis template domain, the nucleic acid synthesis template domain is 5′ of the primer binding site, the primer binding site is 5′ of the spacer domain, the spacer domain is 5′ of the second portion of the gRNA core domain, and a second portion of the gRNA core domain is 5′ of a second ligation sequence, and wherein the first and second portions together form the gRNA core domain, and optionally, a portion of the first ligation sequence is complementary to a portion of the second ligation sequence.

In some embodiments of any one of the aspects described herein, the first linking domain does not form a secondary structure. In some other embodiments, the first linking domain forms at least one secondary structure, (e.g., a hairpin).

In some embodiments of any one of the aspects described herein, the second linking domain does not form a secondary structure. In some other embodiments, the second linking domain forms at least one secondary structure, (e.g., a hairpin).

In some embodiments of any one of the aspects described herein, the pegRNA is a RNA:DNA chimera.

In some embodiments of any one of the aspects described herein, nucleic acid synthesis template domain is a template for an RNA-dependent polymerase (e.g., reverse transcriptase). In some embodiments of any one of the aspects described herein, the nucleic acid synthesis template domain is a template for a DNA-dependent polymerase (e.g., DNA polymerase, such as Bsu polymerase or phiDNA polymerase). In some embodiments of any one of the aspects described herein, at least a part of the nucleic acid synthesis template domain comprises a sequence substantially complementary to a region downstream of a nick region in a second strand of the double-stranded target nucleic acid. In some embodiments of any one of the aspects described herein, the nucleic acid synthesis template domain and the primer binding site are directly adjacent to each other. In some embodiments of any one of the aspects described herein, the nucleic acid synthesis template domain is positioned 5′ to the primer binding site.

In some embodiments of any one of the aspects described herein, the one or more nucleotide changes comprises insertions of one or more nucleotides, substitutions of one or more nucleotides, deletions of one or more nucleotides, or a combination of any such nucleotide changes, as compared to the double-stranded target DNA sequence.

In some embodiments of any one of the aspects described herein, the primer binding site is from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, from 7 to 17 nucleotides, or from 50 nucleotides to 300 nucleotides in length. In some embodiments of any one of the aspects described herein, the primer binding site comprises a sequence having 100% complementarity to a region upstream of the nick site in the second strand of the double-stranded target nucleic acid.

In some embodiments of any one of the aspects described herein, the spacer domain is from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, from 7 to 17 nucleotides in length, or from 20 nucleotide to 200 nucleotides. In some embodiments of any one of the aspects described herein, the spacer domain comprises a sequence having 100% complementarity to the first strand of the double-stranded target nucleic acid, or the spacer domain comprises a sequence having one or more (e.g., 1, 2, 3, 4, or 5) mismatches with the first strand of the double-stranded target nucleic acid.

In some embodiments of any one of the aspects described herein, the gRNA core domain comprises one or more secondary structures. In some embodiments of any one of the aspects described herein, the gRNA core domain comprises at least one (e.g., two, three or more) hairpins. In some embodiments of any one of the aspects described herein, the gRNA core domain comprises a nucleotide sequence having at least 80% identity to a sequence selected from the group consisting of:

(SEQ ID NO: 604)
GTTTCAGAGCTATGCTGGAAACAGCATAGCAAGTTGAAATAAGGCTAGT
CCGTTATCAACTTGAAAAAGTGGCACCGAGTCGGTGC;
and
(SEQ ID NO: 572)
GTTTTAGAGCTAGAAATAGCAAGTTAAAATAAGGCTAGTCCGTTATCAA
CTTGAAAAAGTGGGACCGAGTCGGTCC

In some embodiments of any one of the aspects described herein, the gRNA core domain comprises a nucleotide sequence having 1, 2, 3, 4, 5 or more mutations relative to SEQ ID NO: 1 or 572.

In some embodiments of any one of the aspects described herein, the pegRNA comprises an RNA-binding protein recruitment domain. In some embodiments of any one of the aspects described herein, the pegRNA does not comprise an RNA-binding protein recruitment domain. In some embodiments of any one of the aspects described herein, the RNA-binding protein recruitment domain is positioned 3′ to the primer binding site. In some embodiments of any one of the aspects described herein, the RNA-binding protein recruitment domain is positioned 5′ to the primer binding site. In some embodiments of any one of the aspects described herein, the RNA-binding protein recruitment domain is positioned 3′ to the spacer. In some embodiments of any one of the aspects described herein, the RNA-binding protein recruitment domain is positioned 5′ to the spacer. In some embodiments of any one of the aspects described herein, the RNA-binding protein recruitment domain is an aptamer sequence. In some embodiments of any one of the aspects described herein, the aptamer sequence is a MS2 aptamer sequence.

In some embodiments of any one of the aspects described herein, the pegRNA is circularized. In some embodiments of any one of the aspects described herein, the pegRNA comprises a first portion of the gRNA core domain at one of the 5′-end or the 3′-end, and a second portion of the gRNA core domain at the other of the 5′-end or the 3′-end, and wherein the first and second portions together form the gRNA core domain.

In some embodiments of any one of the aspects described herein, the pegRNA comprises a first ribozyme and a first ligation sequence positioned 3′ to the first ribozyme at 5′-end, and a second ribozyme and a second ligation sequence positioned 3′ to the second ribozyme at the 3′-end, and wherein a portion of the first ligation sequence is complementary to a portion of the first ribozyme and a portion of the second ligation sequence is complementary to a portion of the second ribozyme, wherein a portion of the first ligation sequence is complementary to a portion of the second ligation sequence; and wherein the portion of the first ligation sequence complementary to the portion of the first ribozyme is complementary to the portion of the second ligation sequence complementary to the portion of the second ribozyme. In some embodiments of any one of the aspects described herein, each of the first ribozyme and the second ribozyme comprises a sequence that may be cleaved to produce a 5′-OH end and a 2′,3′-cyclic phosphate end. In some embodiments of any one of the aspects described herein, each of the first and the second ribozyme is independently selected from the group consisting of Hammerhead, Hairpin, Hepatitis Delta Virus (“HDV”), Varkud Satellite (“VS”), Vg1, glucosamine-6-phosphate synthase (“glmS”), Twister, Twister Sister, Hatchet, Pistol ribozymes, engineered synthetic ribozymes, or derivatives thereof. In some embodiments of any one of the aspects described herein, each of the first and the second ribozyme is, independently, a split ribozyme or ligand-activated ribozyme derivative. In some embodiments of any one of the aspects described herein, the first ribozyme is a P3 Twister ribozyme and the second ribozyme is a P1 Twister ribozyme. In some embodiments of any one of the aspects described herein, each of the first ligation sequence and the second ligation sequence are substrates for an RNA ligase. In some embodiments of any one of the aspects described herein, each of the first ligation sequence and the second ligation sequence comprise a portion of a tRNA exon sequence or derivative thereof. In some embodiments of any one of the aspects described herein, the RNA ligase is RtcB.

In some embodiments of any one of the aspects described herein, the nucleic acid programmable DNA binding protein has nickase activity. In some embodiments of any one of the aspects described herein, the nucleic acid programmable DNA binding protein is an RNA guided DNA-binding protein, optionally the nucleic acid programmable DNA binding protein is a CRISPR Cas enzyme, an Argonaute protein, an obligate mobile element guided activity (OMEGA) enzyme, a RuVC nucleases, or a homolog, ortholog or variant thereof. In some embodiments of any one of the aspects described herein, the nucleic acid programmable DNA binding protein is selected from the group consisting of: Cas9 (also known as CsnI and CsxI2), Cas1, Cas100, Cas12a (Cpf1), Cas12b, Cas12b1 (C2c1), Cas12b2, Cas12c (C2c3), Cas12d (CasY), Cas12e (CasX), Cas13a (C2c2), Cas13b (C2c6), Cas13c (C2c7), Cas1B, Cas2, Cas3, Cas4, Cas5, Cas6, Cas7, Cas8, CasI, CasIB, CasIO, Cmr1, Cmr3, Cmr4, Cmr5, Cmr6, Cpf1, Csa5, Csa5, CsaX, Csb1, Csb2, Csb3, Csc1, Csc2, C2c5, C2c8, C2c9, C2c10, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, Ago1, Ago2, Ago3, Ago4, Fz TnpB, Fz, IS110 family recombinases (e.g., IS621, and homologs, orthologs and variants thereof. In some embodiments of any one of the aspects described herein, the nucleic acid programmable DNA binding protein is a Cas9. In some embodiments of any one of the aspects described herein, the nucleic acid programmable DNA binding protein is a mutated Cas9, optionally the mutated Cas9 comprises a dead HNH domain or a dead RuVC domain, and/or the mutated Cas9 is shorter than a wildtype Cas9. In some embodiments of any one of the aspects described herein, the nucleic acid programmable DNA binding protein is Cas9 nickase (nCas9).

In some embodiments of any one of the aspects described herein, the nucleic acid modifying enzyme is a polymerase, an RNA deaminase, an RNA methylase, an RNA demethylase, a retrotransposon or an integrase fused with a polymerase. In some embodiments of any one of the aspects described herein, the nucleic acid modifying enzyme is a polymerase. In some embodiments of any one of the aspects described herein, the polymerase is an RNA-dependent polymerase (e.g., reverse transcriptase). In some embodiments of any one of the aspects described herein, the reverse transcriptase is a reverse transcriptase from a retrovirus or a retrotransposon. In some embodiments of any one of the aspects described herein, the reverse transcriptase is a Moloney-Murine Leukemia Virus reverse transcriptase (M-MLV RT) or a variant of M-MLV RT. In some embodiments of any one of the aspects described herein, the polymerase is a DNA-dependent polymerase (e.g., DNA polymerase, such as Bsu polymerase or phiDNA polymerase). In some embodiments of any one of the aspects described herein, the nucleic acid modifying enzyme lacks nuclease activity.

In some embodiments of any one of the aspects described herein, the pegRNA comprises at least one nucleic acid modification. In some embodiments of any one of the aspects described herein, the pegRNA comprises at least one nucleic acid modification selected from the group consisting of modified internucleoside linkages, modified nucleobases, modified sugars, and any combinations thereof.

In some embodiments of any one of the aspects described herein, the nucleic acid programmable DNA binding protein is not attached or tethered to the nucleic acid modifying enzyme. In some embodiments of any one of the aspects described herein, the nucleic acid programmable DNA binding protein is attached or tethered to the nucleic acid modifying enzyme. In some embodiments of any one of the aspects described herein, the nucleic acid programmable DNA binding protein and the nucleic acid modifying enzyme are comprised in a fusion protein.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1G show Tornado (“Twister-optimized-ribozyme for durable overexpression”) enabled circularization of pegRNA improves base-substitution on a plasmid reporter. FIG. 1A depicts the steps required for Tornado to generate circular pegRNA and alter transition (TAG->TGG) base-substitution on eGFP containing stop-codon. FIG. 1B, linear and Tornado pegRNA were coupled with PE2 to direct eGFP gain-of-function (GoF) and data was analyzed upon gating for mCherry and eGFP positive-cells. FIG. 1C is the histogram representation of the eGFP channel. FIG. 1D shows a comparison of cpegRNA against epegRNA using PE2 in 293T cells. Mean±s.d. of n=3 independent biological replicates. FIG. 1E shows a further comparison of cpegRNA against epegRNA using PEmax in 293T cells. Mean±s.d. of n=3 independent biological replicates. FIG. 1F shows accessing cpegRNA against epegRNA in HeLa cells using PE2. Mean±s.d. of n=3 independent biological replicates. FIG. 1G shows further accessing of cpegRNA and epegRNA in HeLa cells using PEmax system. Mean±s.d. of n=3 independent biological replicates.

FIGS. 2A-2D show engineering of split PE system using cpegRNA. FIG. 2A is a general depiction of the split PE utility using circularized pegRNA. FIG. 2B is an illustration of the pegRNA counterparts coupled with the split PE system. FIG. 2C shows a comparison of PE2 and split PE system against linear and cpegRNA counterparts using the wild-type EGFP construct as an internal control. Mean±s.d. of n=3 independent biological replicates. FIG. 2D shows RT-PCR verification of the linear (e)pegRNA and cpegRNA using circular and linear primer pairs in HeLa cells. Mean±s.d. of n=3 independent biological replicates.

FIGS. 3A-3F show circularized and rotated-pegRNA enabled site-directed insertion and base-substitution within HEK3 genomic loci. FIGS. 3A and 3B show results of inducing 12 types of base modification in HEK3 loci of HEK 293T and HeLa cells using PEmax and circularized pegRNA that bears functionally verified spacer, PBS and RTT domains (2). Mean±s.d. of n=3 independent biological replicates. FIGS. 3C and 3D show a examination of long-range editing in HEK3 loci of HEK 293T and HeLa cells using PE2max and circularized pegRNA that bears 34-nt RTT domain (2). Mean±s.d. of n=3 independent biological replicates. FIGS. 3E and 3F show circularized and rotated-pegRNA enabled site-directed insertion and base-substitution within HEK3 genomic loci. Rotated-pegRNA architecture.

FIGS. 4A-4G show a comparison of circularized cpegRNA to engineered pegRNAs across multiple genomic loci. FIG. 4A is an illustration of the engineered pegRNA and circularized cpegRNA system. FIGS. 4B-4D show results of inducing FLAG-tag insertion within DMNT1 (FIG. 4B), RUNX (FIG. 4C) and VEGF (FIG. 4D) loci using linear (engineered) pegRNA and circularized cpegRNA counterparts in HEK 293T cells. Mean±s.d. of n=3 independent biological replicates. FIGS. 4E-4G shows comparison of the FLAG-tag insertion within DMNT1 (FIG. 4E), RUNX (FIG. 4F) and VEGF (FIG. 4G) loci using linear engineered pegRNA and circularized cpegRNA system in HeLa cells. Mean±s.d. of n=3 independent biological replicates.

FIG. 5 shows sample gating using no pegRNA control construct.

FIG. 6 shows the rotated-pegRNA was not able to edit alterations in HEK3 loci of HeLa cells.

FIG. 7 is a schematic representation of lentiviral based cpegRNA screen against a panel of known disease mutations. The lentiviral library is used to screen for cpegRNA functionality against mutational targets that are supplied downstream of the construct. The integration of the library into the genome, hence, provides a way to infer for the edits using a one-step PCR amplification of the genomic loci. Sample library information is provided in Table 4. The library is adopted from exiting lentiviral screen.

FIG. 8 is a schematic showing coupling of tracrRNA and crRNA within a single rotated pegRNA transcript. In this strategy, the pegRNA is designed to hybridize with its 5′ and 3′ ends around the typical stem formed between the crRNA and tracrRNA. The nicked position can be varied within the tetraloop in addition to stem loop 1, 2 and 3 of the Cas9 gRNA scaffold. The ends of the pegRNA are protected by the CRISPR/Cas enzyme, while the rest of the transcript mimics the structure of the circularized pegRNAs.

FIG. 9 is a schematic depicting use of circularized rotated-pegRNA for prime-editing. The circularization of the rotated pegRNA can be achieved within the tetraloop region of the Cas9 sgRNA scaffold. The circularization event can improve PE functionality due to overcoming possible degradation on the 5′ and 3′ tetraloop domains that are important for PE functionality.

FIG. 10 is a schematic depicting the use of DNA Polymerase (e.g. Phi29 and Bsu) with Rotated-pegRNA for prime-editing. The rotated pegRNA can be made as a DNA:RNA chimera to accommodate DNA polymerase enabled genomic modification. Instead of the RTT domain, the pegRNA can bear DNA Polymerase Template (DPT) that can be synthesized as DNA instead of RNA. The utility of DNA within the rotated pegRNA assures that the genomic synthesis does not extend past the DPT DNAtemplate, which omits possible synthesis of the tracrRNA domain within the genome. The 5′ and 3′ ends of rotated pegRNA can be synthesized with 2′O-methyl groups and phosphorothioate bonds on the first and last three nucleotides. The DNA polymerase mRNA can be supplied either in fusion to Cas9 nickase or supplied as a separate transcript.

FIG. 11 is a schematic depicting use of DNA Polymerase (e.g. Phi29 and Bsu) with Circularized Rotated-pegRNA for prime-editing. The circularized DNA:RNA chimeric Rotated pegRNA can be used to enhance the stability of the pegRNA for DNA polymerase enabled genomic modifications.

FIG. 12 depicts the utility of the cpegRNA in Engineered virus-like particles (eVLPs). The eVLPs provide a payload for transient expression of Prime Editor and pegRNA that bears MS2 in the tetraloop domain. The eVLPs utilize fusion between Gag and MCP domain to install MS2 bearing pegRNA within the payload. The PE is embedded in the payload via engineered protease domain fused to Gag domains that bear Nuclear Export Signal (NES) to assure that the Gag domains are not delivered to nucleus with PE protein. The transient expression of the PE and pegRNA within eVLPs require prolong stability since the cargo must be generated via transduction in human cells and stored prior to delivery in vivo applications. The utility of the cpegRNA can enhance eVLPs functionality due to its higher stability.

FIGS. 13A-13C depict Tornado pegRNA coupled with PEmax provides for site-directed insertion and base-substitution within functionally verified HEK3 loci in 293T cells. FIG. 13A depicts 12 types of base modification from position +1 to +8 of the HEK3 loci were examined using Tornado pegRNA via functionally verified spacer, PBS and RTT domains (2). Mean±s.d. of n=3 independent biological replicates. FIG. 13B depicts examination of long-range PE editing in a verified HEK3 loci using Tornado pegRNA with 34-nt RTT domain (2). Mean±s.d. of n=3 independent biological replicates. FIG. 13C shows a comparison of FLAG-tag insertion within HEK3 loci using Tornado and linear pegRNA counterparts in 293T cells (2). Mean±s.d. of n=3 independent biological replicates.

FIGS. 14A-14C depict coupling of Tornado pegRNA with split PE2 system. FIG. 14A is a general depiction of the constructs used in the split PE system that either consists of PE2, nCas9, MCP-MMLV and MMLV that are combinatorially coupled to Tornado pegRNA, Tornado MS2 pegRNA and linear pegRNA counterparts. FIG. 14B is depiction of the mechanism that couples (Tornado) pegRNA with the split PE system to drive eGFP GoF. FIG. 14C shows results of use of PE2 and split PE system against linear and Tornado pegRNA counterparts. Mean±s.d. of n=3 independent biological replicates.

FIG. 15 is schematic showing coupling of tracrRNA and crRNA within a single quasi-circularized pegRNA transcript. In this strategy, the pegRNA is designed to hybridize with its 5′ and 3′ ends around the typical stem formed between the crRNA and tracrRNA. The nicked position can be varied within the tetraloop in addition to stem loop 1, 2 and 3 of the Cas9 gRNA scaffold. The ends of the pegRNA are protected by the CRISPR/Cas enzyme, while the rest of the transcript mimics the structure of the circularized pegRNAs.

FIG. 16 shows sample library preparation.

FIG. 17 is a schematic showing cpegRNA PE2max, 34-nt RT Template in 293T HEK3 cells +12 G to C. A distribution of identified alleles around the cleavage site for the sgRNA GGCCCAGACTGAGCACGTGA (SEQ ID NO: 15). Nucleotides are indicated by unique colors (A=green, C=red, G=yellow, T=purple). Substitutions are shown in bold font. Red rectangles highlight inserted sequences. Horizontal dashed lines indicate deleted sequences. The vertical dashed line indicates the predicted cleavage site. Figure discloses SEQ ID NOS 608-612, respectively, in order of appearance.

### DETAILED DESCRIPTION

In one aspect of any of the embodiments, described herein is a prime editing guide RNA (pegRNA). The pegRNA comprises: (a) a spacer domain; (b) a gRNA core (scaffold) domain capable of associating with a nucleic acid programmable DNA binding protein (napDNAbp); (c) a nucleic acid synthesis template (RTT) domain; and (d) a primer binding site (PBS) comprising a sequence substantially complementary to a region upstream of the region complementary to the nucleic acid synthesis template domain in the second strand of the double-stranded target nucleic acid. In some embodiments of any one of the aspects described herein, the pegRNA is circularized. As used herein, the term “circularized pegRNA” or “circular pegRNA”, abbreviated “cpegRNA”, refers to a pegRNA the 3′- and 5′-end of which are protected against degradation, e.g., degradation by an exonuclease. For example, the cpegRNA lacks one or both of a 3′-OH and/or 5′-OH. In some embodiments, the 3′-end and the 5′-end of the pegRNA are covalently linked to each other.

The term, “prime editing guide RNA” or “pegRNA” as used herein, refers to a guide RNA molecule that encodes the crRNA-tracrRNA fused to a primer binding site (PBS) and a nucleic acid synthesis template (e.g., a polymerase template) nucleic acid sequence.

In some embodiments of any one of the aspects described herein, the pegRNA comprises a first portion of the gRNA core domain at one of the 5′-end or the 3′-end, and a second portion of the gRNA core domain at the other of the 5′-end or the 3′-end, and wherein the first and second portions together form the gRNA core domain.

In some embodiments of any one of the aspects described herein, the pegRNA comprises a first ribozyme and a first ligation sequence positioned 3′ to the first ribozyme at 5′-end, and a second ribozyme and a second ligation sequence positioned 3′ to the second ribozyme at the 3′-end, and wherein a portion of the first ligation sequence is complementary to a portion of the first ribozyme and a portion of the second ligation sequence is complementary to a portion of the second ribozyme, wherein a portion of the first ligation sequence is complementary to a portion of the second ligation sequence; and wherein the portion of the first ligation sequence complementary to the portion of the first ribozyme is complementary to the portion of the second ligation sequence complementary to the portion of the second ribozyme.

It is noted that the spacer domain, the gRNA core domain, the nucleic acid synthesis template domain, and the primer binding site of the pegRNA can be located or oriented independently of each other in the pegRNA. In some embodiments, the spacer domain is 5′ of the scaffold domain. In some embodiments, the spacer domain is 3′ of the scaffold domain. In some embodiments, the spacer domain is 5′ of the RTT domain. In some embodiments, the spacer domain is 3′ of the RTT domain. In some embodiments, the spacer domain is 5′ of the PBD. In some embodiments, the spacer domain is 3′ of the PBS. In some preferred embodiments, 3′-end of the spacer domain is linked directly to 5′-end of the gRNA core domain, i.e., a RTT domain and/or a PBS is not present between the 3′-end of the spacer domain and the 5′-end of the gRNA core domain.

In some embodiments, the scaffold domain is 5′ of the nucleic acid synthesis template domain. In some embodiments, the scaffold domain is 3′ of the RTT domain. In some embodiments, the scaffold domain is 5′ of the PBS. In some embodiments, the scaffold domain is 3′ of the PBS. In some preferred embodiments, 3′-end of the scaffold domain is linked directly to 5′-end of the RTT domain, i.e., a spacer domain and/or a PBS is not present between the 3′-end of the gRNA core domain and the 5′-end of the nucleic acid synthesis template domain.

In some embodiments, the RTT domain is 5′ of the PBS. In some embodiments, the RTT domain is 3′ of the PBS. In some preferred embodiments, 3′-end of the RTT domain is linked directly to 5′-end of the PBS, i.e., a spacer domain and/or a gRNA core domain is not present between the 3′-end of the nucleic acid synthesis template domain and the 5′-end of the PBS.

In some embodiments of any of the aspects, the nucleic acid synthesis template domain and the primer binding site are directly adjacent to each other. In some embodiments of any of the aspects, the nucleic acid synthesis template domain is positioned 5′ to the primer binding site.

In some embodiments of any one of the aspects, the pegRNA comprises in a 5′ to 3′ orientation: the spacer domain, the gRNA core domain, the nucleic acid synthesis template domain, and the primer binding site.

It is noted that a circularized pegRNA does not have a 5′- or 3′-end per se. Thus, in the context of a circularized pegRNA, reference to a 5′ to 3′ orientation means starting with the first domain listed and proceeding in a 5′ to 3′ direction along the sequence. Thus, the spacer domain is located 5′ of the gRNA core, the gRNA core is located 5′ of the nucleic acid synthesis template domain, and the nucleic acid synthesis template domain is located 5′ of the primer binding site in a pegRNA that comprises in a 5′ to 3′ orientation: the spacer domain, the gRNA core domain, the nucleic acid synthesis template domain, and the primer binding site. Stated in another way, the PBS is located 3′ of the nucleic acid synthesis template domain, the nucleic acid synthesis template domain is located 3′ of the gRNA core, and the gRNA core is located 3′ of the spacer domain in a pegRNA that comprises in a 5′ to 3′ orientation: the spacer domain, the gRNA core domain, the nucleic acid synthesis template domain, and the primer binding site.

In some embodiments of any one of the aspects, the pegRNA comprises in a 5′ to 3′ orientation: a first portion of the gRNA core domain, the nucleic acid synthesis template domain, the primer binding site, the spacer domain, and a second portion of the gRNA core domain, and wherein first and second portions together form the gRNA core domain. Stated in another way, the pegRNA comprises a first portion of the gRNA core domain located 5′ of the nucleic acid synthesis template domain, the nucleic acid synthesis template domain located 5′ of the primer binding site, the primer binding site located 5′ of the spacer domain, and the spacer domain located 5′ of a second portion of the gRNA core domain.

In some embodiments of any one of the aspects, the pegRNA comprises in a 5′ to 3′ orientation: a first ligation sequence, a first portion of the gRNA core domain, the nucleic acid synthesis template domain, the primer binding site, the spacer domain, a second portion of the gRNA core domain, and a second ligation sequence, and wherein the first and second portions together form the gRNA core domain. For example, the pegRNA comprises in a 5′ to 3′ orientation: a first ligation sequence, a first portion of the gRNA core domain, the nucleic acid synthesis template domain, the primer binding site, the spacer domain, a second portion of the gRNA core domain, and a second ligation sequence, and wherein the first and second portions together form the gRNA core domain, and wherein a portion of the first ligation sequence is complementary to a portion of the second ligation sequence. Stated in another way, the pegRNA comprises a first ligation sequence located 5′ of a first portion of the gRNA core domain, the first portion of the gRNA core domain located 5′ of the nucleic acid synthesis template domain, the nucleic acid synthesis template domain located 5′ of the primer binding site, the primer binding site located 5′ of the spacer domain, the spacer domain located 5′ of a second portion of the gRNA core domain, and the second portion of the gRNA core domain located 5′ of a second ligation sequence, and optionally the first and second portions together form the gRNA core domain, and/or a portion of the first ligation sequence is complementary to a portion of the second ligation sequence.

In some embodiments of any one of the aspects, the pegRNA comprises in a 5′ to 3′ orientation: a first ribozyme, a first ligation sequence, a first portion of the gRNA core domain, the nucleic acid synthesis template domain, the primer binding site, the spacer domain, a second portion of the gRNA core domain, a second ligation sequence, and a second ribozyme, and optionally the first and second portions together form the gRNA core domain, and/or a portion of the first ligation sequence is complementary to a portion of the second ligation sequence. Stated in another way, the pegRNA comprises a first ribozyme located 5′ of a first ligation, the first ligation sequence located 5′ of a first portion of the gRNA core domain, the first portion of the gRNA core domain located 5′ of the nucleic acid synthesis template domain, the nucleic acid synthesis template domain located 5′ of the primer binding site, the primer binding site located 5′ of the spacer domain, the spacer domain located 5′ of a second portion of the gRNA core domain, the second portion of the gRNA core domain located 5′ of a second ligation sequence, the second ligation sequence located 5′ of the second ribozyme, and optionally the first and second portions together form the gRNA core domain, and/or a portion of the first ligation sequence is complementary to a portion of the second ligation sequence.

In some embodiments of any of the aspects, the pegRNA comprises a first ribozyme and a first ligation sequence positioned 3′ to the first ribozyme at 5′-end, and a second ribozyme and a second ligation sequence positioned 3′ to the second ribozyme at the 3′-end, and wherein a portion of the first ligation sequence is complementary to a portion of the first ribozyme and a portion of the second ligation sequence is complementary to a portion of the second ribozyme, wherein a portion of the first ligation sequence is complementary to a portion of the second ligation sequence; and wherein the portion of the first ligation sequence complementary to the portion of the first ribozyme is complementary to the portion of the second ligation sequence complementary to the portion of the second ribozyme.

In some embodiments of any of the aspects, the pegRNA is a RNA:DNA chimera. By an “RNA:DNA chimera” is meant the pegRNA comprises both ribonucleotides (e.g., RNA) and deoxyribonucleotides (e.g., DNA).

### Spacer Domain

Embodiments of the various aspects described herein include a spacer domain. As used herein, a “spacer domain” refers to a nucleotide sequence recognizing a target sequence. A spacer domain is also referred to a guide sequence herein. Generally, the spacer domain comprises a nucleotide sequence substantially complementary to the desired target site, e.g., a nucleotide sequence complementary to the non-target strand, i.e., the non-edit strand of the double-stranded target nucleic acid.

In some embodiments, the spacer domain comprises a nucleotide sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%) complementarity to the non-edit strand of the target nucleic acid. In some embodiments, the spacer domain comprises a nucleotide sequence having at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%) complementarity to non-edit strand of the target nucleic acid. In some embodiments, the spacer domain comprises a nucleotide sequence having at least 90% (e.g., at least 91%, at least 92%, at least 93%, at least 94%, at least 95%, at least 96%, at least 97%, at least 98%, at least 99%) complementarity to the non-edit strand of the target nucleic acid. In some embodiments, the spacer domain comprises a nucleotide sequence having at least 95% (e.g., at least 96%, at least 97%, at least 98%, at least 99%) complementarity to the non-edit strand of the target nucleic acid. In some embodiments, the spacer domain comprises a nucleotide sequence having 100% (i.e., complete) complementarity to the non-edit strand of the target nucleic acid.

Without wishing to be bound by a theory, conditional mismatches between the spacer domain sequence and the non-edit strand of the target nucleic acid can allow the nicking event to be solely programmed against a mutation variant of the target rather than the wild-type counterpart. In other words, the spacer can be fully cognate to the mutation but not the wild-type sequence. In some instances, the mismatch in the seed region of the Cas9 gRNA spacer can allow for conditional nicking event. Thus, in some embodiments, the spacer domain comprises a nucleotide sequence having at least 1 (e.g., 2, 3, 4, 5 or more) mismatches with the non-edit strand of the target nucleic acid.

The length of the spacer domain can range from a few nucleotides to 100 s of nucleotides. For example, the spacer domain can be at least 3, at least 4, at least 5, at least 6, at least 7, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 1 at least 167, at least 168, at least 169, at least 170, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250 or more nucleotides in length. In some embodiments of any one of the aspects, the spacer domain is from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, or from 7 to 17 nucleotides in length. In some preferred embodiments, the spacer domain is from about 15 to about 25 nucleotides in length.

In some embodiments, the spacer domain is from about 15 to about 250 nucleotides in length. For example, from about 20 to about 200 nucleotides in length.

In some embodiments of any of the aspects, the spacer domain comprises a sequence having 100% complementarity to a first strand (e.g., the non-PAM or non-edit strand) of a double-stranded target nucleic acid, e.g., DNA. In some embodiments of any one of the aspects described herein, the spacer domain comprises one or more, e.g., 1, 2, 3, 4, 5 or more mismatches with the first strand (e.g., the non-PAM or non-edit strand) of a double-stranded target nucleic acid, e.g., DNA.

gRNA Core Domain

Embodiments of the various aspects descried herein include a gRNA core domain. As used herein, the term “gRNA core domain” or “gRNA scaffold” refers to a nucleotide sequence that is capable of associating with or binding with a nucleic acid programmable DNA binding protein. (napDNAbp). The gRNA core domain is also referred to as a scaffold domain herein.

The gRNA core domain can be at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 1 at least 167, at least 168, at least 169, at least 170, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250 or more nucleotides in length. In some embodiments, the gRNA core domain is from about 50 nucleotides to about 250 nucleotides in length. For example, the gRNA core domain is from about 60 nucleotides to about 220 nucleotides, from about 70 nucleotides to about 200 nucleotides, from about 75 nucleotides to about 175 nucleotides, from about 80 nucleotides to about 150 nucleotides, from about 90 nucleotides to about 125 nucleotides, from about 70 nucleotides to about 120 nucleotides, from about 60 nucleotides to about 100 nucleotides, or from about 80 nucleotides to about 100 nucleotides in length.

A nucleotide sequence of the gRNA core domain can comprise one or more secondary structures, e.g., for associating or binding with a napDNAbp. For example, the gRNA core domain can comprise a nucleotide sequence that capable of forming one or more hairpin or stem-loop structures. In some embodiments, the gRNA core domain comprises 1, 2, 3, 4 or 5 hairpin or stem-loop structures. For example, the gRNA core domain comprises 2, 3 or 4 hairpin or stem-loop structures. In some embodiments, the gRNA core domain comprises only 3 hairpin or stem-loop structures. In some embodiments, the gRNA core domain comprises only 4 hairpin or stem-loop structures. In some preferred embodiments, the gRNA core domain comprises only 2 hairpin or stem-loop structures.

In some embodiments, the gRNA core domain comprises a hairpin or stem-loop structure comprising an internal loop and/or bulge loop. For example, the gRNA core domain comprises a hairpin or stem-loop structure comprising an internal symmetric or asymmetric loop. In some embodiments, the gRNA core domain comprises a bulge loop. In some embodiments, the gRNA core domain comprises a multi-branch loop. It is noted that a mismatch in a stem or double-stranded region of a hairpin or stem-loop structure can be considered an internal symmetric loop or bulge.

In some embodiments, the gRNA core domain comprises a hairpin or stem-loop structure comprising from about 20 nucleotides, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, or about 65 nucleotides. For example, the gRNA core domain comprises a hairpin or stem-loop structure comprising from about 15 to about 45, from about 20 to about 40 or from about 25 to 35 nucleotides. In some embodiments, the gRNA core domain comprises a hairpin or stem-loop structure comprising from about about 25 to 35 (e.g., 26, 27, 28, 29, 30, 31, 32, 33, or 34, such as 28, 29, 30, 31 or 32, preferably 29, 30 or 31) nucleotides and wherein the hairpin or stem-loop structure further comprises an internal symmetric or asymmetric bulge or loop, preferably an asymmetric internal bulge or loop.

In some embodiments, the gRNA core domain comprises a hairpin or stem-loop structure comprising from about 8 nucleotides, about 9, about 10, about 11, about 12, about 13, about 14, about 15, about 16, about 17, about 18, about 19, about 20, about 21, about 22, about 23, about 24, or about 25 nucleotides. For example, the gRNA core domain comprises a hairpin or stem-loop structure comprising from about 10 to about 20, from about 12 to about 18 or from about 13 to about nucleotides. In some embodiments, the gRNA core domain comprises a hairpin or stem-loop structure comprising from about about 10 to about 20 (e.g., 11, 12, 13, 14, 15, 16, 17, 18 or 19, such as 13, 14, 15, 16, or 17, preferably 14, 15 or 16) nucleotides and wherein the hairpin or stem-loop structure further comprises an internal symmetric or asymmetric bulge or loop, preferably an asymmetric internal bulge or loop. In some embodiments, the gRNA core domain comprises a hairpin or stem-loop structure comprising from about about 10 to about 20 (e.g., 11, 12, 13, 14, 15, 16, 17, 18 or 19, such as 13, 14, 15, 16, or 17, preferably 14, 15 or 16) nucleotides and wherein the hairpin or stem-loop structure further comprises both an internal symmetric bulge or loop (e.g., a mismatch) and an internal asymmetric bulge or loop.

In some embodiments, the gRNA core domain comprises a hairpin or stem-loop structure comprising from about about 10 to about 20 (e.g., 11, 12, 13, 14, 15, 16, 17, 18 or 19, such as 13, 14, 15, 16, or 17, preferably 14, 15 or 16) nucleotides and wherein the hairpin or stem-loop structure does not comprise an internal loop or bulge.

In some embodiments, the gRNA core domain comprises a hairpin or stem-loop structure comprising from about about 7 to about 17 (e.g., 8, 9, 10, 11, 12, 13, 14, 15, or 16, such as 10, 11, 12, 13, or 14, preferably 11, 12 or 13) nucleotides.

In some embodiments, the gRNA core domain comprises comprising a hairpin or stem-loop structure comprising from about about 25 to 35 (e.g., 26, 27, 28, 29, 30, 31, 32, 33, or 34, such as 28, 29, 30, 31 or 32, preferably 29, 30 or 31) nucleotides; a second hairpin or stem-loop structure comprising from about about 10 to about 20 (e.g., 11, 12, 13, 14, 15, 16, 17, 18 or 19, such as 13, 14, 15, 16, or 17, preferably 14, 15 or 16) nucleotides; a third hairpin or stem-loop structure comprising from about about 7 to about 17 (e.g., 8, 9, 10, 11, 12, 13, 14, 15, or 16, such as 10, 11, 12, 13, or 14, preferably 11, 12 or 13) nucleotides; and a fourth hairpin or stem-loop structure comprising from about about 10 to about 20 (e.g., 11, 12, 13, 14, 15, 16, 17, 18 or 19, such as 13, 14, 15, 16, or 17, preferably 14, 15 or 16) nucleotides.

In some embodiments, the gRNA core domain comprises a nucleotide sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity to a sequence selected from the group consisting of:

(SEQ ID NO: 604)
GTTTCAGAGCTATGCTGGAAACAGCATAGCAAGTTGAAATAAGGCTAGT
CCGTTATCAACTTGAAAAAGTGGCACCGAGTCGGTGC;
(SEQ ID NO: 572)
GTTTTAGAGCTAGAAATAGCAAGTTAAAATAAGGCTAGTCCGTTATCAA
CTTGAAAAAGTGGGACCGAGTCGGTCC;
and
(SEQ ID NO: 571)
GUUUUAGAGCUAGAAAUAGCAAGUUAAAAUAAGGCUAGUCCGUUAUCAA
CUUGAAAAAGUGGCACCGAGUCGGUGC.

In some embodiments, the gRNA core domain comprises a nucleotide sequence having at least 85%, e.g., at least 86%, at least 87%, at least 88%, at least 89%, at least 90%, at least 91%, at least 92%, at least 93%, at least 94%, at least 95%, at least 96%, at least 97%, at least 98%, at least 99% identity to one of SEQ ID NO: 1, 571, and 572. For example, the gRNA core domain comprises a nucleotide sequence having at least 90%, e.g., at least 91%, at least 92%, at least 93%, at least 94%, at least 95%, at least 96%, at least 97%, at least 98%, at least 99% identity to one of SEQ ID NO: 1, 571, and 572. In some embodiments, the gRNA core domain comprises a nucleotide sequence having at least 95%, at least 96%, at least 97%, at least 98%, at least 99% identity to one of SEQ ID NO: 1, 571, and 572. In some embodiments, the gRNA core domain comprises a nucleotide sequence having 100%, i.e., complete identity to one of SEQ ID NO: 1, 571, and 572.

### Nucleic Acid Synthesis Template (RTT) Domain

Embodiments of the various aspects descried herein include a nucleic acid synthesis template (RTT) domain. As used herein, the term “nucleic acid synthesis template domain” refers to the region or portion of a pegRNA that is utilized as a template strand by a polymerase of a prime editor to encode a 3′ single-strand DNA flap that contains the desired edit and which then, through the mechanism of prime editing, replaces the corresponding endogenous strand of double-stranded nucleic acid at the target site. Generally, the nucleic acid synthesis template domain encodes (by the polymerase) a single-stranded DNA which, in turn, has been designed to be (a) homologous with the target nucleic acid to be edited, and (b) which comprises at least one desired nucleotide change (e.g., a transition, a transversion, a deletion, or an insertion) to be introduced or integrated into the target nucleic acid, e.g., DNA.

Generally, the nucleic acid template comprising both an edit of interest (e.g., an “edit template domain” and regions of homology (i.e., “homology arm domains”) that are homologous with the 5′ ended single stranded strand (DNA) immediately following the nick site on the PAM strand. The edit template domain can be as small as a single nucleotide substitution, or it may be an insertion, an inversion or transversion. The edit template domain can also include a deletion, which can be engineered by encoding homology arm that contains a desired deletion. It is noted that a sequence of the edit template domain comprises one or more nucleotide changes compared to the second strand (i.e., the edit-strand) of the target nucleic acid. Thus, a sequence of the edit template domain is not fully complementary to the edit-strand. For example, the edit template domain comprises a sequence that has less than 100% (e.g., less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, or even) identity to the edit strand of the target nucleic acid.

The length of the edit template domain can range from a few nucleotides to 1000 s of nucleotides. For example, the nucleic acid synthesis template domain can be 1 nucleotide or longer. For example, the edit template can be at least 2, at least 3, at least 4, at least 5, at least 6, at least 7, at least at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least at least 67, at least 68, at least 69, at least 70, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 1 at least 167, at least 168, at least 169, at least 170, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 275, at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775, at least 800, at least 825, at least 850, at least 875, at least 900, at least 925, at least 950, at least 975, at least 1 k, at least 1.5 k, at least 2 k, at least 2 k, at least 2.5 k, at least 3 k, at least 3.5 k, at least 4 k, at least 4.5 k, at least 5 k, at least 5.5 k, at least 6 k, at least 6.5 k, at least 7 k, at least 7.5 k, at least 8 k, at least 8.5 k, at least 91 k, at least 9.5 k, at least 10 k or more nucleotides in length. In some embodiments, the edit template can be a single nucleotide.

Generally, the homology arm domain comprises a nucleotide sequence substantially complementary to the edit-strand of the target nucleic acid. In some embodiments, the homology arm domain comprises a nucleotide sequence substantially complementary to a region of the edit-strand that is downstream of the region of the first strand (i.e., the non-edit strand) of the target nucleic acid that is complementary to the spacer domain. In some embodiments, the homology arm domain comprises a nucleotide sequence substantially complementary to a region of the edit-strand that is downstream of the region of the first strand (i.e., the non-edit strand) of the target nucleic acid that is complementary to the spacer domain. In some embodiments, the homology arm domain comprises a nucleotide sequence substantially complementary to a region of the edit-strand that overlaps with the region of the first strand (i.e., the non-edit strand) of the target nucleic acid that is complementary to the spacer domain.

In some embodiments of any of the aspects, the nucleic acid synthesis template comprises a sequence substantially complementary to a region downstream of a nick region in the second strand (i.e., edit strand) of the double-stranded target nucleic acid.

In some embodiments, the homology arm domain comprises a nucleotide sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%) complementarity to the edit strand of the target nucleic acid. In some embodiments, the homology arm domain comprises a nucleotide sequence having at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%) complementarity to edit strand of the target nucleic acid. In some embodiments, the homology arm domain comprises a nucleotide sequence having at least 90% (e.g., at least 91%, at least 92%, at least 93%, at least 94%, at least 95%, at least 96%, at least 97%, at least 98%, at least 99%) complementarity to the edit strand of the target nucleic acid. In some embodiments, the homology arm domain comprises a nucleotide sequence having at least 95% (e.g., at least 96%, at least 97%, at least 98%, at least 99%) complementarity to the edit strand of the target nucleic acid. In some embodiments, the homology arm domain comprises a nucleotide sequence having 100% (i.e., complete) complementarity to the edit strand of the target nucleic acid

Length of each homology arm can range from a few nucleotides to 10 s of nucleotides. For example, each homology arm independently can be at least 3, at least 4, at least 5, at least 6, at least 7, at least at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 or more nucleotides in length. In some embodiments, each homology arm independently be from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, or from 7 to 17 nucleotides in length.

In some embodiments, the nucleic acid synthesis template can also include a sequence or secondary structure that causes termination of polymerase activity.

In some embodiments, the nucleic acid synthesis template domain comprises an edit template flanked by homology arms on both sides. Stated in another way, the nucleic acid synthesis template domain comprises in series, a first homology arm, an edit template, and a second homology arm.

The length of the nucleic acid synthesis template domain comprising the edit template domain and the homology arm domain can range from a few nucleotides to 1000 s of nucleotides. For example, the nucleic acid synthesis template domain can be at least 3, at least 4, at least 5, at least 6, at least 7, at least at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least at least 67, at least 68, at least 69, at least 70, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 1 at least 167, at least 168, at least 169, at least 170, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 275, at least 300, at least 325, at least 350, at least 375, at least 400, at least 425, at least 450, at least 475, at least 500, at least 525, at least 550, at least 575, at least 600, at least 625, at least 650, at least 675, at least 700, at least 725, at least 750, at least 775, at least 800, at least 825, at least 850, at least 875, at least 900, at least 925, at least 950, at least 975, at least 1 k, at least 1.5 k, at least 2 k, at least 2 k, at least 2.5 k, at least 3 k, at least 3.5 k, at least 4 k, at least 4.5 k, at least 5 k, at least 5.5 k, at least 6 k, at least 6.5 k, at least 7 k, at least 7.5 k, at least 8 k, at least 8.5 k, at least 91 k, at least 9.5 k, at least 10 k or more nucleotides in length. For example, the nucleic acid synthesis template domain of the pegRNA can be from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, or from 7 to 17 nucleotides in length.

In some embodiments, the nucleic acid synthesis template domain comprises a sequence substantially complementary to a region downstream of the region of the first strand (that is complementary to the spacer domain (e.g., the target strand (i.e., the non-PAM strand or the non-edit strand))) in a second strand (e.g., the non-target strand (i.e., the PAM strand or the edit strand)) of the double-stranded target nucleic acid, and wherein the sequence of the nucleic acid synthesis template domain comprises one or more nucleotide changes compared to the double-stranded target nucleic acid, i.e., compared to the non-target strand (i.e., the PAM strand or the edit strand).

In some embodiments of any of the aspects, the nucleic acid synthesis template domain is a template or substrate for a nucleic acid modifying enzyme. For example, the nucleic acid synthesis template domain is a template or substrate for a polymerase (e.g., an RNA-dependent polymerase or a DNA-dependent polymerase), or an enzyme that can edit a nucleotide or ribonucleotide (e.g., adenosine deaminases, ADAR family proteins, cytidine deaminases, APOBEC family proteins, and PPR proteins), those that can methylate RNA (e.g., domains from m6A methyltransferase factors such as METTL3, METTL4, METTL14, or WTAP), those that can demethylate RNA (e.g., human alkylation repair homolog 5 or ALKBH5), those that can affect splicing (e.g., the RS-rich domain of SRSF1, the Gly-rich domain of hnRNP A1, the alanine-rich motif of RBM4, or the proline-rich motif of DAZAP1), those that can activate translation (e.g., eIF4E, N-terminal domain of the YT521-B homolog domain family protein 1 (YTHDF1, a cytoplasmicm6A reader protein that recruits the translation machinery) and other translation initiation factors, a domain of the yeast poly(A)-binding protein or GLD2), those that can repress translation (e.g., Pumilio or FBF PUF proteins, deadenylases, or CAF1) and those that can affect RNA stability (e.g., tristetraprolin (TTP) or domains from UPF1, EXOSC5, and STAU1).

In some embodiments, the nucleic acid synthesis template domain is template for an RNA-dependent polymerase (e.g., reverse transcriptase).

In some other embodiments of any of the aspects, the nucleic acid synthesis template domain is a template for a DNA-dependent polymerase (e.g., DNA polymerase).

In some embodiments of any of the aspects, the nucleic acid synthesis template domain and the primer binding site are directly adjacent to each other.

In some embodiments of any of the aspects, the nucleic acid synthesis template domain is positioned 5′ to the primer binding site.

### Primer Binding Site (PBS)

Embodiments of the various aspects descried herein include a primer binding site (PBS). As used herein the “primer binding site” or “the PBS” refers to a nucleotide sequence in the pegRNA that hybridizes to a single-strand sequence (e.g., the primer sequence) that is formed after nicking of the target sequence, e.g., the edit strand by the napDNAbp. Without wishing to be bound by a theory, when the napDNAbp, e.g., Cas9 nickase nicks one strand of the double-stranded target nucleic acid, a 3′-ended single-stranded flap is formed, which serves as a primer sequence that anneals to the primer binding site on the pegRNA to prime the polymerase, e.g., a reverse transcriptase or a DNA polymerase. The PBS serves to bind the pegRNA to the primer sequence that is formed after nicking of the target sequence, e.g., the edit strand by the napDNAbp. It is noted that the primer binding site does not generally form a part of the template that is used by the polymerase (e.g., reverse transcriptase or DNA polymerase) to encode the resulting 3′ single-strand flap that includes the desired edit.

Generally, the PBS comprises a sequence substantially complementary to a second stand (i.e., edit strand) of the target nucleic acid. For example, the PBS comprises a sequence substantially complementary to a region upstream of the region complementary to the nucleic acid synthesis template domain in the second strand (i.e., edit strand) of the target nucleic acid. In some embodiments of any one of the aspects, the PBS comprises a sequence substantially complementary to a region upstream of the nick site in the second strand (i.e., the edit strand) of the target nucleic acid.

The PBS can comprise a nucleotide sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%) complementarity to the edit strand of the target nucleic acid. In some embodiments, the PBS comprises a nucleotide sequence having at least 85% (e.g., at least 86%, at least 87%, at least 88%, at least 89%, at least 90%, at least 91%, at least 92%, at least 93%, at least 94%, at least 95%, at least 96%, at least 97%, at least 98%, at least 99%) complementarity to edit strand of the target nucleic acid. In some embodiments, the PBS comprises a nucleotide sequence having at least 90% (e.g., at least 91%, at least 92%, at least 93%, at least 94%, at least 95%, at least 96%, at least 97%, at least 98%, at least 99%) complementarity to the edit strand of the target nucleic acid. In some embodiments, the PBS comprises a nucleotide sequence having at least 95% (e.g., at least 96%, at least 97%, at least 98%, at least 99%) complementarity to the edit strand of the target nucleic acid. In some embodiments, the PBS comprises a nucleotide sequence having 100% (i.e., complete) complementarity to the edit strand of the target nucleic acid.

The length of the PBS can range from a few nucleotides to 100 s of nucleotides. For example, the nucleic acid synthesis template domain can be 1 nucleotide or longer. For example, the edit template can be at least 2, at least 3, at least 4, at least 5, at least 6, at least 7, at least at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least at least 67, at least 68, at least 69, at least 70, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 1 at least 167, at least 168, at least 169, at least 170, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250 or more nucleotides in length. For example, the primer binding site is from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, or from 7 to 17 nucleotides in length.

### Linking Domain

Various domains of the pegRNA, e.g., the spacer domain, the scaffold domain, the RTT domain and/or the PBS can be linked to each other via a linking domain. As used herein, a “linking domain” in reference to polynucleotide, e.g., a pegRNA refers to a sequence of one or more nucleotides used to link two domains together, e.g., as a separator between two domains. A linking domain can comprise one or more nucleotides.

The length of each linking domain can range from a few nucleotides to 100 s of nucleotides. For example, each linking domain independently can be a single nucleotide or longer. For example, the each linking domain independently can be at least 2, at least 3, at least 4, at least 5, at least 6, at least 7, at least at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least at least 67, at least 68, at least 69, at least 70, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 1 at least 167, at least 168, at least 169, at least 170, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250 or more nucleotides in length. In some embodiments, each linking domain is from about 5 to about 150, from about 10 to about 100, from about 15 to about 75, or from about 20 to about 50 nucleotides in length.

A nucleotide sequence of a linking domain can comprise one or more secondary structures. For example, a linking domain can comprise a nucleotide sequence that capable of forming one or more hairpin or stem-loop structures. In some embodiments, a linking domain comprises a hairpin or stem-loop structure comprising an internal loop and/or bulge loop. For example, the linking domain comprises a hairpin or stem-loop structure comprising an internal symmetric or asymmetric loop.

In some other embodiments, a linking domain does not comprise a secondary structure, e.g., a hairpin or stem-loop structure.

In some embodiments, a linking domain comprises a poly A sequence. For example, the linking domain comprises the sequence (A)ₙ, where n is an integer from 4 to 25, e.g., n is 5, 6, 7, 8, 9, 10, 11, 12, 13, 14, 15, 16, 17, 18, 19, 20, 21, 22, 23, or 24,

In some embodiments of any one of the aspects, the pegRNA comprises a linking domain between the primer binding site and the spacer domain. For example, the pegRNA comprises a linking domain between the primer binding site and the spacer domain, and wherein the linking domain does not form a secondary structure. In another non-limiting example, the pegRNA comprises a linking domain between the primer binding site and the spacer domain, and wherein the linking domain can form secondary structure, e.g., at least one hairpin or stem-loop structure.

In some embodiments, the linking domain between the between the primer binding site and the spacer domain is at least 1, at least 2, at least 3, at least 4, at least 5, at least 6, at least 7, at least 8, at least 9, at least 10, at least 11, at least 12, at least 13, at least 14, at least 15 in length, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 80, at least 55, at least 90, at least 95, at least 100, at least 150, at least 200, at least 250 or more nucleotides in length.

The linking domain between the between the primer binding site and the spacer domain can comprise a nucleotide sequence that capable of forming one or more hairpin or stem-loop structures. In some embodiments, the linking domain between the between the primer binding site and the spacer domain comprises a hairpin or stem-loop structure comprising an internal loop and/or bulge loop. For example, the linking domain comprises a hairpin or stem-loop structure comprising an internal symmetric or asymmetric loop. In some embodiments, the linking domain between the between the primer binding site and the spacer domain does not comprise a secondary structure, e.g., a hairpin or stem-loop structure. In some embodiments, the linking domain between the between the primer binding site and the spacer domain comprises a polyA sequence.

In some embodiments of any one of the aspects, the pegRNA comprises a linking domain between a first portion of the gRNA core domain and a second portion of the gRNA core domain, optionally, the first portion of the gRNA core domain and the second portion of the gRNA core domain together form the full gRNA core domain. For example, the pegRNA comprises a linking domain between the first portion of the gRNA core domain and the second portion of the gRNA core domain, and wherein the linking domain does not form a secondary structure. In another non-limiting example, the pegRNA comprises a linking domain between the first portion of the gRNA core domain and the second portion of the gRNA core domain, and wherein the linking domain can form secondary structure, e.g., at least one hairpin or stem-loop structure.

In some embodiments, the linking domain between the first portion of the gRNA core domain and the second portion of the gRNA core domain is at least 1, at least 2, at least 3, at least 4, at least 5, at least 6, at least 7, at least 8, at least 9, at least 10, at least 11, at least 12, at least 13, at least 14, at least 15 in length, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 80, at least 55, at least 90, at least 95, at least 100, at least 150, at least 200, at least 250 or more nucleotides in length.

The linking domain between the first portion of the gRNA core domain and the second portion of the gRNA core domain can comprise a nucleotide sequence that capable of forming one or more hairpin or stem-loop structures. In some embodiments, the linking domain between the first portion of the gRNA core domain and the second portion of the gRNA core domain comprises a hairpin or stem-loop structure comprising an internal loop and/or bulge loop. For example, the linking domain comprises a hairpin or stem-loop structure comprising an internal symmetric or asymmetric loop. In some embodiments, the linking domain between the first portion of the gRNA core domain and the second portion of the gRNA core domain does not comprise a secondary structure, e.g., a hairpin or stem-loop structure. In some embodiments, the linking domain between the first portion of the gRNA core domain and the second portion of the gRNA core domain comprises a poly A sequence.

### Ribozymes and Ligation Sequences

In some embodiments of any of the aspects, the pegRNA comprises a first ribozyme and second ribozyme. As used herein, the term “ribozyme” refers to an RNA sequence that hybridizes to a complementary sequence in a substrate RNA and cleaves the substrate RNA in a sequence specific manner at a substrate cleavage site. Typically, a ribozyme contains a catalytic region flanked by two binding regions. The ribozyme binding regions hybridize to the substrate RNA, while the catalytic region cleaves the substrate RNA at a substrate cleavage site to yield a cleaved RNA product. The nucleotide sequence of the ribozyme binding regions can be completely complementary or partially complementary to the substrate RNA sequence with which the ribozyme hybridizes

In some embodiments of any one of the aspects, each of the first ribozyme and the second ribozyme comprise a sequence that can be cleaved to produce a 5′-OH end and a 2′,3′-cyclic phosphate end. In accordance with this embodiment, each of the first ribozyme and the second ribozyme is a self-cleaving ribozyme. Self-cleaving ribozymes are known in the art and are characterized by distinct active site architectures and divergent, but similar, biochemical properties. The cleavage activities of self-cleaving ribozymes are highly dependent upon divalent cations, pH, and base-specific mutations, which can cause changes in the nucleotide arrangement and/or electrostatic potential around the cleavage site. Some exemplary self-cleaving ribozymes include, but are not limited to, Hammerhead, Hairpin, Hepatitis Delta Virus (“HDV”), Neurospora Varkud Satellite (“VS”), Vgl, glucosamine-6-phosphate synthase (glmS), Twister, Twister Sister, Hatchet, Pistol, and engineered synthetic ribozymes, and derivatives thereof. See, for example, Weinberg et al., “New Classes of Self-Cleaving Ribozymes Revealed by Comparative Genomics Analysis,” Nat. Chem. Biol. 11(8): 606-610 (2015); Lee et al., “Structural and Biochemical Properties of Novel Self-Cleaving Ribozymes,” Molecules 22(4):E678 (2017); Harris et al., “Biochemical Analysis of Pistol Self-Cleaving Ribozymes,” RNA 21(11):1852-8 (2015); Roth et al., “A Widespread Self-Cleaving Ribozyme Class is Revealed by Bioinformatics,” Nature Chem. Biol. 10(1):56-60 (2014); and Gebetsberger et al., “Unwinding the Twister Ribozyme: from Structure to Mechanism,” WIREs RNA 8(3):e1402 (2017), the contents of all of which are incorporated herein by reference in their entireties.

In some embodiments of any one of the aspects, one of the first ribozyme and second ribozyme is a Twister ribozyme, a Twister Sister ribozyme or a Pistol ribozyme. For example, one of the first ribozyme and second ribozyme can be a P3 Twister ribozyme. In another non-limiting example one of the first ribozyme and second ribozyme can be a P1 Twister ribozyme.

In some embodiments of any one of the aspects, the first ribozyme and second ribozyme independently are a Twister ribozyme, a Twister Sister ribozyme or a Pistol ribozyme. For example, one of the first ribozyme and second ribozyme can be a P1 Twister ribozyme and the other can be a P3 Twister ribozyme.

In some embodiments of any one of the aspects, each of the first and the second ribozyme is, independently, a split ribozyme or ligand-activated ribozyme derivative.

Embodiments of the various aspects described herein include a ligation sequence. For example, the pegRNA comprises a first ligation sequence and a second ligation sequence. As used herein, the term “ligation sequence” refers to a sequence complementary to another sequence, which enables the formation of Watson-Crick base pairing to form suitable substrates for ligation by a ligase, e.g., an RNA ligase, such as RtcB. In some embodiments, each of the first ligation sequence and the second ligation sequence comprise a portion of a tRNA exon sequence or derivative thereof. The first ligation sequence and the second ligation sequence may each, independently, comprise at least 1, 2, 3, 4, 5, 6, 7, 8, 9, 10, 11, or 12 additional nucleotides to promote base-pairing with each other. In some embodiments of the various aspects descried herein, the ligation sequences are substrates for an RNA ligase, such as RtcB.

The ligation sequences can assist in circularization of the pegRNA, and/or protect the pegRNA from degradation. Without wishing to be bound by a theory, this can enhance expression of the pegRNA. While it is thought that pegRNA of the present invention could circularize without the ligation sequences, and such an invention is hereby contemplated, the ligation sequences are also believed to cause the pegRNA ends to more efficiently come together for the RNA ligase (e.g., RtcB). In other words, the ligation sequences can help draw proper 5′ and 3′ ends of the pegRNA closer to each other to assist in the circularization of the pegRNA.

Length of a ligation sequence can range from a few nucleotides to 10 s of nucleotides. For example, each homology arm independently can be at least 3, at least 4, at least 5, at least 6, at least 7, at least at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 or more nucleotides in length.

### Adaptor Protein Recruitment Domain

In some embodiments of any one of the various aspects described herein, the pegRNA can comprise an adaptor protein recruitment domain. As used herein, an “adaptor protein recruitment domain” refers to a nucleotide sequence that can associate or bind with an adaptor protein, which adaptor protein can recruit a nucleic acid modifying enzyme to the pegRNA. Thus, without wishing to be bound by a theory, the adaptor protein recruitment domain can be used to recruit a nucleic acid modifying enzyme to the pegRNA. Exemplary adaptor proteins include, but are not limited to MS2, PP7, Qβ, F2, GA, fr, JP501, M12, R17, BZ13, JP34, JP500, KUI, M11, MX1, TW18, VK, SP, FI, ID2, NL95, TW19, AP205, ϕCb5, ϕCb8r, ϕCb12r, ϕCb23r, 7s, and PRR1. The adaptor protein recruitment domain is also referred to as an RNA-binding protein recruitment domain herein.

In some embodiments of any one of the aspects described herein, the adaptor protein recruitment domain (i.e., the RNA-binding protein recruitment domain) is an aptamer. As used herein, the term “aptamer” refers to a nucleic acid molecule that binds with high affinity and specificity to a target. It is noted that aptamers can be single-stranded, partially single-stranded, partially double-stranded, or double-stranded nucleotide sequences. In some embodiments, the adaptor protein recruitment domain is a MS2 aptamer, e.g., the adaptor protein recruitment domain comprises the sequence CGGCAUCAGUUCGGC (SEQ ID NO: 605). In some embodiments, the adaptor protein recruitment domain is a PP7 aptamer, e.g., the adaptor protein recruitment domain comprises the AUAUGG.

In some embodiments of any one of the various aspects described herein, the pegRNA does not comprise an adaptor protein recruitment domain. For example, the pegRNA does not comprise an MS2 aptamer or PP7 aptamer sequence.

When present, the adaptor protein recruitment domain (i.e., the RNA-binding protein recruitment domain) can be positioned anywhere in the pegRNA. For example, the adaptor protein recruitment domain can be positioned 3′ to the primer binding site. In another non-limiting example, the adaptor protein recruitment domain can be positioned 5′ to the primer binding site. In yet another non-limiting examples, the adaptor protein recruitment domain can be positioned 3′ to the spacer. In still some other non-limiting examples, the adaptor protein recruitment domain can be positioned 5′ to the spacer. In yet still some other examples, the adaptor protein recruitment domain can be positioned 3′ to the gRNA core domain. In some cases, the adaptor protein recruitment domain is positioned 5′ to the gRNA core domain. In some examples, the adaptor protein recruitment domain can be positioned between the primer binding site and the spacer. In some other examples, the adaptor protein recruitment domain can be positioned between the spacer and the gRNA core. In yet other examples, the adaptor protein recruitment domain can be positioned between the gRNA core and the PBS or the RTT domain.

### Nucleic Acid Modifying Enzyme

Embodiments of the various aspects descried herein include a nucleic acid modifying enzyme. As used herein, a “nucleic acid modifying enzyme” refers to an enzyme or a functional fragment thereof capable of modifying nucleic acids. Nucleic acid modifying enzymes include DNA modifying enzymes and RNA modifying enzymes. The term also includes nucleic acid polymerases such as RNA polymerases, DNA polymerases, retrotransposons, and integrases.

Exemplary nucleic acid modifying enzymes include, but are not limited to, polymerases (e.g., RNA polymerases and DNA polymerases) and active fragments thereof, retrotransposons and active fragments thereof, integrases and active fragments thereof, and enzymes that can edit a nucleotide or ribonucleotide (e.g., adenosine deaminases, ADAR family proteins, cytidine deaminases, APOBEC family proteins, and PPR proteins), those that can methylate RNA (e.g., domains from m6A methyltransferase factors such as METTL3, METTL4, METTL14, or WTAP), those that can demethylate RNA (e.g., human alkylation repair homolog 5 or ALKBH5), those that can affect splicing (e.g., the RS-rich domain of SRSF1, the Gly-rich domain of hnRNP A1, the alanine-rich motif of RBM4, or the proline-rich motif of DAZAP1), those that can activate translation (e.g., eIF4E, N-terminal domain of the YT521-B homolog domain family protein 1 (YTHDF1, a cytoplasmicm6A reader protein that recruits the translation machinery) and other translation initiation factors, a domain of the yeast poly(A)-binding protein or GLD2), those that can repress translation (e.g., Pumilio or FBF PUF proteins, deadenylases, or CAF1) and those that can affect RNA stability (e.g., tristetraprolin (TTP) or domains from UPF1, EXOSC5, and STAU1), and homologs, orthologs and variants thereof.

In some embodiments of any one of the aspects described herein, the nucleic acid modifying enzyme is an RNA-dependent polymerase. As used herein, the term “RNA-dependent polymerase” refers to an enzyme that produces a polynucleotide sequence (DNA or RNA), complementary to a pre-existing template polyribonucleotide (RNA). The RNA-dependent polymerase can be either an RNA-dependent RNA polymerase or an RNA-dependent DNA polymerase. The RNA-dependent polymerase can be either an RNA viral polymerase or replicase or an RNA-dependent cellular polymerase. Exemplary RNA polymerases include, but are not limited to, Exemplary RNA polymerases include, but are not limited to, viral RNA polymerases such as T7 RNA polymerase, T3 polymerase, SP6 polymerase, and KII polymerase; eukaryotic RNA polymerases such as RNA polymerase 1, RNA polymerase II, RNA polymerase III, RNA polymerase IV, and RNA polymerase V; archaeal RNA polymerases, and homologs, orthologs and variants thereof. Additional exemplary RNA polymerases are descried in U.S. Pat. No. 8,460,910, contents of which are incorporated herein by reference in their entireties.

In some embodiments, the RNA dependent polymerase is a reverse transcriptase. Exemplary reverse transcriptases include, but are not limited to, reverse transcriptases from Murine Moloney Leukemia Virus (MMLV), Avian Myelomatosis Virus (AMV), and/or Human Immunodeficiency Virus (HIV), telomerase reverse transcriptases such as (hTERT), SuperScript™ III, SuperScript™ IV reverse transcriptase, and ProtoScript™ II reverse transcriptase and homologs, orthologs and variants thereof. Some specific exemplary reverse transcriptases include, but are not limited to, Murine Moloney Leukemia virus (MLV) reverse transcriptase, murine leukemia virus (MLV) reverse transcriptase, Avian Myeloblastosis Virus (AMV) reverse transcriptase, Respiratory Syncytial Virus (RSV) reverse transcriptase, Equine Infectious Anemia Virus (EIAV) reverse transcriptase, Rous-associated Virus-2 (RAV2) reverse transcriptase, SUPERSCRIPT II reverse transcriptase, SUPERSCRIPT I reverse transcriptase, THERMOSCRIPT reverse transcriptase and MMLV RNase H⁻ reverse transcriptases, and homologs, orthologs and variants thereof.

In some preferred embodiments, the nucleic acid modifying enzyme is a MMLV reverse transcriptase (MMLV RT) or a homolog, ortholog or variant of MMLV RT. In some preferred embodiments, the nucleic acid modifying enzyme is MMLV RT.

In some embodiments of any one of the aspects described herein, the nucleic acid modifying enzyme is a DNA-dependent polymerase. As used herein, the term “DNA-dependent polymerase” refers to an enzyme that produces a polynucleotide sequence (DNA or RNA), complementary to a pre-existing template polydeoxyribonucleotide (DNA). The DNA-dependent polymerase may be either a DNA-dependent RNA polymerase or a DNA-dependent DNA polymerase.

Exemplary DNA polymerases include, but are not limited to bacterial DNA polymerases, eukaryotic DNA polymerases, archaeal DNA polymerases, viral DNA polymerases and phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, μ, and k, as well as the RevI polymerase (terminal deoxycytidyl transferase) and terminal deoxynucleotidyl transferase (TdT). Viral DNA polymerases include T4 DNA polymerase, phi-29 DNA polymerase, GA-I, phi-29-like DNA polymerases, PZA DNA polymerase, phi-15 DNA polymerase, CpI DNA polymerase, Cp7 DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; the heterodimeric DNA polymerase DP1/DP2, and homologs, orthologs and variants thereof.

In some embodiments, the DNA polymerase is Bsu or phi29DNA. It is noted that Bsu and phi29 DNA polymerases can operate near room temperature at isothermal condition.

In some embodiments, the nucleic acid modifying enzyme is an RNA deaminase, an RNA methylase, an RNA demethylase, or a homolog, ortholog, or variant thereof.

It is noted that retrotransposons including R2 variants as well as variants that can reverse-transcribe gene sized cargo can be used as a nucleic acid modifying enzyme. Thus, in some embodiments, the nucleic acid modifying enzyme is a retrotransposon or a homolog, ortholog or variant thereof. Exemplary retrotransposons are described, for example, in US patent publication US20240035008 and in Zhang, X., Van Treeck, B., Horton, C. A. et al. Harnessing eukaryotic retroelement proteins for transgene insertion into human safe-harbor loci. Nat Biotechnol 43, 42-51 (2025), contents of all of which are incorporated herein by reference in their entireties.

In some embodiments, the nucleic acid modifying enzyme is an integrase. For example, the nucleic acid modifying enzyme is an integrase fused with a polymerase, e.g. fused with a reverse transcriptase. Exemplary integrases are described in Fell, C. W., Schmitt-Ulms, C., Tagliaferri, D. V. et al. Precise kilobase-scale genomic insertions in mammalian cells using PASTE. Nat Protoc (2024), contents of all of which are incorporated herein by reference in their entireties.

In some embodiments, the nucleic acid modifying enzyme is attached to or tethered with an adaptor protein. For example, the nucleic acid modifying enzyme and the adaptor protein are in the form of a fusion protein. As used herein, the term “adaptor protein” means a protein having the ability to specifically bind to a certain molecule (e.g., adaptor protein recruitment domain), and which permits binding of a first (e.g., a nucleic acid modifying enzyme) and second component (e.g., pegRNA), either by modifying or interacting with (e.g., binding) the first (e.g., a nucleic acid modifying enzyme) or the second component (e.g., pegRNA) component such that it is then able to bind the other directly, or by binding both of the first and second components, thereby creating a bridge such that the first and second components are present in the same complex. Some exemplary adaptor proteins include, but are not limited to MS2, PP7, QP, F2, GA, fr, JP501, M12, R17, BZ13, JP34, JP500, KUI, M11, MX1, TW18, VK, SP, FI, ID2, NL95, TW19, AP205, ϕCb5, ϕCb8r, ϕCb12r, ϕCb23r, 7s, and PRR1. In some embodiments, the adaptor protein is MS2.

In some preferred embodiments, the nucleic acid modifying enzyme is not attached to or tethered with an adaptor protein

### Nucleic Acid Programmable DNA Binding Protein

As used herein, the term “nucleic acid programmable DNA binding protein” or “napDNAbp,” of which Cas9 is an example, refer to a proteins which use RNA:DNA hybridization to target and bind to specific sequences in a DNA molecule. Each napDNAbp is associated with at least one guide nucleic acid (e.g., guide RNA such as pegRNA), which localizes the napDNAbp to a DNA sequence that comprises a DNA strand (i.e., a target strand) that is complementary to the guide nucleic acid, or a portion thereof (e.g., the protospacer of a guide RNA such as the spacer domain of the pegRNA). In other words, the guide nucleic-acid (e.g., the pegRNA) “programs” the napDNAbp (e.g., Cas9 or equivalent) to localize and bind to a complementary sequence. Accordingly, in some embodiments, the nucleic acid programmable DNA binding protein is an RNA guided DNA-binding protein.

Without being bound by a theory, the binding mechanism of a napDNAbp-guide RNA complex, in general, can include a step of forming an R-loop whereby the napDNAbp induces the unwinding of a double-strand DNA target, thereby separating the strands in the region bound by the napDNAbp. The guide RNA protospacer (e.g., the spacer domain of the pegRNA) then hybridizes to the “target strand.” This displaces a “non-target strand” that is complementary to the target strand, which can form the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand.

In some embodiments of any of the aspects, the napDNAbp binding protein of the prime is not attached or tethered to the nucleic acid modifying enzyme. Alternatively, in some embodiments of any of the aspects, the napDNAbp is attached or tethered to the nucleic acid modifying enzyme.

In some embodiments, the napDNAbp is a CRISPR Cas enzyme. As used herein, the term “Cas enzyme,” or the term “CRISPR Cas enzyme,” refers to any of the enzymes involved in a CRISPR system for any form of bacteria or archaea, which are nucleases capable of cutting a specific nucleic acid target by complexing with a guide RNA. Some exemplary CRISPR Cas enzymes include, but are not limited to, Cas9 (also known as CsnI and CsxI2), Cas1, Cas100, Cas12a (Cpf1), Cas12b, Cas12b1 (C2c1), Cas12b2, Cas12c (C2c3), Cas12d (CasY), Cas12e (CasX), Cas13a (C2c2), Cas13b (C2c6), Cas13c (C2c7), Cas1B, Cas2, Cas3, Cas4, Cas5, Cas6, Cas7, Cas8, CasI, CasIB, CasIO, Cmr1, Cmr3, Cmr4, Cmr5, Cmr6, Cpf1, Csa5, Csa5, CsaX, Csb1, Csb2, Csb3, Csc1, Csc2, C2c5, C2c8, C2c9, C2c10, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues, variants and modified versions thereof.

In some embodiments of any one of the aspects described herein, the napDNAbp has nickase activity, i.e., the napDNAbp is a nickase. As used herein, the term “nickase” refers to a napDNAbp (e.g., a Cas9) that can cleave only one strand of the duplex in a double-stranded nucleic acid molecule. Stated in another way, a “nickase” refers to a napDNAbp having only a single nuclease activity that cuts only one strand of a target DNA, rather than both strands. Thus, a nickase (e.g., nCas9) does not leave a double-strand break.

In some embodiments, the napDNABP is a Cas9. In some embodiments, a Cas9 nuclease comprises one or more mutations that partially impair or inactivate the DNA cleavage domain. For example, the Cas9 comprising one or more mutations has a dead HNH domain or a dead RuVC domain. Exemplary napDNAbp with partially impaired or inactivated DNA cleavage domain include “Cas9 nickase” (“nCas9”) and a deactivated Cas9 having no nuclease activities (“dead Cas9” or “dCas9”). In some preferred embodiments, the napDNAbp is a mutated Cas9 such as a Cas9 nickase. Exemplary mutated Cas9 enzymes are described, for example, in PCT publications WO2020191248, WO2020191241, WO2020191243, WO2020191242, WO2020191245, WO2020191171, WO2020191246, WO2020191249, WO2020191153 and WO2020191234, contents of all of which are incorporated herein by reference in their entireties.

It is noted that any direct evolved version of the Cas9 that may bear mutations that improve its catalytic function can be used. In addition, miniaturized version of Cas9 that can be packaged in AAV construct while optionally being fused to a nucleic acid modifying enzyme, e.g., a reverse transcriptase can also be used.

In some embodiments of any one of the aspects described herein, the napDNAbp is a recombinase. Exemplary recombinases include, but are not limited to, IS110 family recombinases (e.g., IS621), RecA, UvsX, RadA, Rad51, DmcI, UvsY, Cre, Flp, Dre, SCre, VCre, Vika, B2, B3, KD, ϕDC31, Bxb1, λ, HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, HbiFm, and homologues thereof, variants thereof and modified versions thereof. For example, the napDNAbp is a IS110 family recombinase (e.g., IS621), or a homolog, ortholog, variant or modified version thereof. IS110 family recombinases are described in Hiraizumi, M., Perry, N. T., Durrant, M. G. et al. Structural mechanism of bridge RNA-guided recombination. Nature 630, 994-1002 (2024), contents of which are incorporated herein by reference in their entireties.

In some embodiments, the napDNAbp is an obligate mobile element guided activity (OMEGA) enzyme. For example, the napDNAbp is TnpB, Fz, Tnpb-IS200/IS605-like protein, a homolog, ortholog, variant or modified version thereof. Exemplary OMEGA enzymes are described in Saito, M., Xu, P., Faure, G. et al. Fanzor is a eukaryotic programmable RNA-guided endonuclease. Nature 620, 660-668 (2023), contents of which are incorporated herein by reference in their entireties.

In some embodiments, the napDNAbp is an argonaute (Ago) protein. For example, the napDNAbp is a DNA-guided argonaute protein. For example, the napDNAbp is Ago1, Ago2, Ago 3, Ago4, PIWI1, PIWI2, PIWI3, PIWI4, or a homolog, ortholog or variant thereof.

### Prime Editing System

In another aspect provided herein is a prime editing system. Generally, the prime editing system comprises a pegRNA described herein or a nucleic acid encoding same, a napDNAbp or a nucleic acid encoding same, and a nucleic acid modifying enzyme or a nucleic acid encoding same. Prime editing system is also referred to as a prime editor herein.

It is noted that the prime editing system can comprise any one of the various pegRNA embodiments described herein. Similarly, the prime editing system can comprise any one of the various napDNAbp embodiments described herein. Furthe, the prime editing system can comprise any one of the various nucleic acid modifying enzyme embodiments described herein.

In some embodiments of the various aspects described herein, prime editing system comprises a pegRNA described herein or a nucleic acid encoding same, a napDNAbp or a nucleic acid encoding same, and a nucleic acid modifying enzyme or a nucleic acid encoding same, and wherein the pegRNA is circularized, the napDNAbp is nCas9, and the nucleic acid modifying enzyme is a reverse transcriptase (e.g., MMLV RT).

In some embodiments of the various aspects described herein, prime editing system comprises a pegRNA described herein or a nucleic acid encoding same, a napDNAbp or a nucleic acid encoding same, and a nucleic acid modifying enzyme or a nucleic acid encoding same, and wherein the napDNAbp is attached to or tethered to the nucleic acid modifying enzyme, optionally the napDNAbp and the nucleic acid modifying enzyme are comprises in a fusion protein.

In some embodiments of the various aspects described herein, prime editing system comprises a pegRNA described herein or a nucleic acid encoding same, a napDNAbp or a nucleic acid encoding same, and a nucleic acid modifying enzyme or a nucleic acid encoding same, and wherein the napDNAbp is not attached to or tethered to the nucleic acid modifying enzyme.

In some embodiments of the various aspects described herein, prime editing system comprises a pegRNA described herein or a nucleic acid encoding same, a napDNAbp or a nucleic acid encoding same, and a nucleic acid modifying enzyme or a nucleic acid encoding same, and wherein the pegRNA does not comprise an adaptor protein recruitment domain, and optionally the pegRNA is circularized.

### Compositions

In another aspect provided herein is a composition comprising a pegRNA described herein or a nucleic acid encoding same. It is noted that the prime editing system can comprise any one of the various pegRNA embodiments described herein.

In some embodiments, the composition further comprises a napDNAbp or a nucleic acid encoding same. It is noted that the prime editing system can comprise any one of the various napDNAbp embodiments described herein.

In some embodiments, the composition further comprises a nucleic acid modifying enzyme or a nucleic acid encoding same. It is noted that the prime editing system can comprise any one of the various nucleic acid modifying enzyme embodiments described herein.

In some embodiments of the various aspects described herein, the composition comprises a pegRNA described herein or a nucleic acid encoding same, a napDNAbp or a nucleic acid encoding same, and a nucleic acid modifying enzyme or a nucleic acid encoding same, and wherein the pegRNA is circularized, the napDNAbp is nCas9, and the nucleic acid modifying enzyme is a reverse transcriptase (e.g., MMLV RT).

In some embodiments of the various aspects described herein, the composition comprises a pegRNA described herein or a nucleic acid encoding same, a napDNAbp or a nucleic acid encoding same, and a nucleic acid modifying enzyme or a nucleic acid encoding same, and wherein the napDNAbp is attached to or tethered to the nucleic acid modifying enzyme, optionally the napDNAbp and the nucleic acid modifying enzyme are comprises in a fusion protein.

In some embodiments of the various aspects described herein, the composition comprises a pegRNA described herein or a nucleic acid encoding same, a napDNAbp or a nucleic acid encoding same, and a nucleic acid modifying enzyme or a nucleic acid encoding same, and wherein the napDNAbp is not attached to or tethered to the nucleic acid modifying enzyme.

In some embodiments of the various aspects described herein, the composition comprises a pegRNA described herein or a nucleic acid encoding same, a napDNAbp or a nucleic acid encoding same, and a nucleic acid modifying enzyme or a nucleic acid encoding same, and wherein the pegRNA doesnot comprise an adaptor protein recruitment domain, and optionally the pegRNA is circularized.

In some embodiments of the various aspects described herein, the composition further comprises a nucleic acid delivery system. As used herein, “nucleic acid delivery system” refers to the composition or components employed to deliver nucleic acids to a desired target, e.g., a cell in vitro, ex vivo and/or in vivo. Exemplary nucleic acid delivery systems include, liposomes, lipid nanoparticles, biolistics, virosomes, polycation or lipid:nucleic acid conjugates and artificial virions. In some embodiments of the various aspects described herein, the nucleic acid delivery system is a virus like particle (VLP), e.g., an engineered VLP.

### Virus Like Particle (VLP)

In some embodiments, the composition comprises a virus like particle (VLP). As used herein, the term “virus-like particle” refers to a structure resembling a virus particle but which has not been demonstrated to be pathogenic. Typically, a virus-like particle does not carry genetic information encoding the proteins of the virus-like particle. In general, virus-like particles lack the viral genome and, therefore, are noninfectious. Also, virus-like particles can often be produced in large quantities by heterologous expression and can be easily purified. Some virus-like particles may contain nucleic acid distinct from their genome. As indicated, a virus-like particle in accordance with the invention is non replicative and noninfectious since it lacks all or part of the viral genome, in particular the replicative and infectious components of the viral genome. A virus-like particle may contain nucleic acid distinct from their genome.

Generally, the VLP is, at a minimum, to a cell membrane-derived membrane component displaying a transmembrane viral envelope protein or a solvent-exposed portion thereof, and a nucleic acid cargo within the membrane component. In some embodiments, the nucleic acid cargo comprises a sequence that corresponds to the viral envelope protein.

A virus-like particle's nucleic acid cargo component can encode the envelope protein and the pegRNA, the napDNAbp and/or the nucleic acid modifying enzyme. In some embodiments, a virus-like particle includes viral-derived structure that permits the packaging of the nucleic acid into the particle, and/or viral-derived structure that permits the introduction of the nucleic acid to a target cell; thus, a virus-like particle can also include a capsid protein or a capsid that permits the packaging of the nucleic acid in a form permitting delivery into a cell. In some embodiments, a virus-like particle does not include viral-derived packaging structures

A typical and preferred embodiment of a virus-like is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAg's have a spherical form of icosahedral symmetry. The term “capsid-like structure” as used herein, refers to a macromolecular assembly composed of viral protein subunits reassembling the capsid morphology in the above defined sense but deviating from the typical symmetrical assembly while maintaining a sufficient degree of order and repetitiveness.

Generally, the VLP comprises a pegRNA described herein or a nucleic acid encoding same. In addition to the pegRNA, the VLP can also comprise a napDNAbp or a nucleic acid encoding same, and/or a nucleic acid modifying enzyme or a nucleic acid encoding same.

In some embodiments, the VLP is an engineered VLP (eVLP). Exemplary engineered VLPs are described, for example, in Banskota et al., Cell 185 (2): 250-265 (2022); Mangeot et al., Nature Communications (1): 1-15 (2019); Campbell, et al., Molecular Therapy 27:151-163 (2019); Campbell, et al., Molecular Therapy, 27 (2019): 151-163; and Mangeot et al Molecular Therapy, 19 (9): 1656-1666 (2011), contents of all of which are incorporated herein by reference in their entireties. Exemplary eVLP are also described, for example, in PCT publication WO2024215652, WO2024254346, contents of all of which are incorporated herein by reference in their entireties.

### Pharmaceutical Composition

In some embodiments, the composition is a pharmaceutical composition. These pharmaceutically acceptable compositions comprise one or more of the various components described herein (e.g., including, but not pegRNA, napDNbp, and nucleic acid modifying enzyme, and complexes comprising same), formulated together with one or more pharmaceutically acceptable carriers (additives) and/or diluents. As described in detail below, the pharmaceutical compositions described herein can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used here, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the composition is in form of a liposome. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. In some embodiments, the liposomes are also specifically targeted, e.g., to direct the protein effector polypeptides (or polynucleotides encoding same), fusion proteins (or polynucleotides encoding same), guide nucleic acids (or polynucleotides encoding same), and/or complexes comprising same to particular cell types.

### Lipid Nanoparticles (LNPs)

In some embodiments, the composition is in form of lipid nanoparticles (NLPs). For example, the composition comprises one or more of the various components described herein (e.g., including, but not limited to, the protein effector polypeptides (or polynucleotides encoding same), fusion proteins (or polynucleotides encoding same), guide nucleic acids (or polynucleotides encoding same), and complexes comprising same) and a cationic lipid. Exemplary cationic lipids include, but are not limited to, N-[1-(2,3-dioleyloxy)propyl-N,N,N-trimethylammonium chloride (DOTMA); N-[1-(2,3-dioleoyloxy)propyl-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1), N1-[2-((1 S)-1-[(3-aminopropyl)amino]-4-[di(3-amino-propyl) aminolbutylc arboxamidoiethylI-3, 4-di [oleyloxy]-benzamide (MVL5); Dioctadecylamido-glycylspermine (DOGS); 3b-[N—(N′,N′-dimethylaminoethyl)carb amoyl] cholesterol (DC-Chol); Dioctadecyldimethylammonium Bromide (DDAB); a Saint lipid (e.g., SAINT-2, N-methyl-4-(dioleyl)methylpyridinium); 1,2-dimyristyloxypropy1-3-dimethylhydroxyethylammonium bromide (DMRIE); 1,2-dioleoy1-3-dimethyl-hydroxyethyl ammonium bromide (DORIE); 1,2-dioleoyloxypropy1-3-dimethylhydroxyethyl ammonium chloride (DORI); Di-alkylated Amino Acid (DILA2) (e.g., C18:1-norArg —C16); Dioleyldimethylammonium chloride (DODAC); 1-p almitoy1-2-oleoyl-sn-glycero-3-ethylpho sphocholine (POEPC); and 1,2-dimyristoleo yl-sn-glycero-3-ethylphosphocholine (MOEPC). In some variations, the condensing agent, e.g. a cationic lipid, is a lipid such as, e.g., Dioctadecyldimethylammonium bromide (DDAB), 1,2-dilinoleyloxy-3-dimethylaminopropane (DLinDMA), 2,2-dilinoley1-4-(2dimethylaminoethyl)-[1,31-dioxolane (DLin-KC2-DMA), heptatriaconta-6,9,28,31-tetraen-19-yl-4-(dimethylamino)butanoate (DLin-MC3-DMA), 1,2-Dioleoyloxy-3-dimethylaminopropane (DODAP), 1,2-Dioleyloxy-3-dimethylaminopropane (DODMA), Morpholinocholesterol (Mo—CHOL), (R)-5-(dimethylamino)pentane-1,2-diy1 dioleate hydrochloride (DODAPen-C1), (R)-5-guanidinopentane-1,2-diy1 dioleate hydrochloride (DOPen-G), and (R)—N,N,N-trimethy1-4,5-bis(oleoyloxy)pentan-1-aminium chloride (DOTAPen). The cationic lipid can comprise 20-90% (mol) of the total lipid present in the lipid nanoparticle. For example, cationic lipid molar content can be 20-70% (mol), 30-60% (mol) or 40-50% (mol) of the total lipid present in the lipid nanoparticle. In some embodiments, cationic lipid comprises from about 50 mol % to about 90 mol % of the total lipid present in the lipid nanoparticle.

In some embodiments, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity. The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of cationic lipid to the neutral lipid ranges from about 2:1 to about 8:1. Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-pho sphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, lysophosphatidylcholine, dilinoleoylphosphatidylcholine, or mixtures thereof. It is to be understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Other examples of non-cationic lipids suitable for use in the lipid nanoparticles include nonphosphorous lipids such as, e.g., stearylamine, dodecylamine, hexadecylamine, acetyl palmitate, glycerolricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid is a phospholipid. In some embodiments, the non-cationic lipid is selected from DSPC, DPPC, DMPC, DLPE, DMPE, DPHyPe, DOPC, POPC, DOPE, and SM. In some preferred embodiments, the non-cationic lipid is DSPC.

In some embodiments, the lipid nanoparticle can further comprise a component, such as a sterol, to provide membrane integrity. The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle. Generally, the component used for providing membrane integrity is non-fusogenic, i.e., a component that does not or substantially does not fuse with a membrane or, if does fuse with a membrane, does not destabilize the membrane. Generally, the component used in the nanoparticles of the invention for providing membrane integrity does not have, or has very little, fusogenic activity at any pH.

One exemplary sterol that can be used in the lipid nanoparticle is cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholestryl hemisuccinate (CHEMS).

In some embodiments, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such asATTA-lipid conjugates), cationic-polymer lipid (CPL) conjugates, and mixtures thereof. In some embodiments, conjugated lipid molecule is a PEG-lipid conjugate, for example, a (methoxy polyethylene glycol)-conjugated lipid. The PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, US2015/0376115 and US2016/0376224, contents of all which are incorporated herein by reference in their entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid is selected from the group consisting N-(Carbonyl-methoxypo1yethy1eneg1yco1n)-1,2-dimyristoyl-sn-glycero-3-phosphoethanolamine (DMPE-PEGₙ, where n is 350, 500, 750, 1000 or 2000), N-(Carbonyl-methoxypolyethyleneglycoln)-1,2-distearoyl-sn-glycero-3-phosphoethanolamine (DSPE-PEGₙ, where n is 350, 500, 750, 1000 or 2000), DSPE-polyglycelin-cyclohexyl-carboxylic acid, DSPE-polyglycelin-2-methylglutar-carboxylic acid, polyethylene glycol-dimyristolglycerol (PEG-DMG), polyethylene glycol-distearoyl glycerol (PEG-DSG), or N-octanoyl-sphingosine-1-{succinyl[methoxy(polyethylene glycol)200011 (C8 PEG2000 Ceramide). In some examples of DMPE-PEGₙ, where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some examples of DSPE-PEG. where n is 350, 500, 750, 1000 or 2000, the PEG-lipid is N-(Carbonyl-methoxypolyethyleneglycol 2000)-1,2-distearoyl-sn-glycero-3-phosphoethanolamine (DSPE-PEG 2,000). In some preferred embodiments, the PEG-lipid is PEG-DMG.

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid.

Generally, the lipid nanoparticles have a mean diameter selected to provide an intended therapeutic effect. Accordingly, in some embodiments, the lipid nanoparticle has a mean diameter from about 30 nm to about 150 nm, more typically from about 50 nm to about 150 nm, more typically about 60 nm to about 130 nm, more typically about 70 nm to about 110 nm, most typically about 85 nm to about 105 nm, and preferably about 100 nm. In some embodiments, the lipid particles that larger in relative size to common nanoparticles and about 150 to 250 nm in size.

Depending on the intended use of the lipid particles, the proportions of the components can be varied and the delivery efficiency of a particular formulation can be measured using, for example, an endosomal release parameter (ERP) assay.

### Protein Nanoparticles

In some embodiments, the composition can be inform of protein nanoparticles. Non-limiting examples of protein nanoparticles include ferritin nanoparticles (see, e.g., Zhang, Y. Int. J. Mol., 12:5406-5421, 2011, incorporated by reference herein), encapsulin nanoparticles (see, e.g., Sutter et al., Nature Struct, and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively.

### Other Exemplary Compostions

In some embodiments, the composition is in form of lipoplexes/polyplexes. Lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²⁺ microcomplexes), polyethylenimine (PEI) (e.g., branched PEI), and poly(L-lysine) (PLL).

In some embodiments, the composition is in form DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42): 14722-5; and Sun W et al, Angew Chem Int Ed Engl. 2015 Oct. 5; 54(41): 12029-33. DNA nanoclew may have a palindromic sequences to be partially complementary to the guide RNA within the IscB polypeptide nuclease:hRNA ribonucleoprotein complex. A DNA nanoclew may be coated, e.g., coated with PEI to induce endosomal escape.

In some embodiments, the composition can comprise one or more of the various components described herein (e.g., including, but not limited to, the protein effector polypeptides (or polynucleotides encoding same), fusion proteins (or polynucleotides encoding same), guide nucleic acids (or polynucleotides encoding same), and complexes comprising same) complexed with gold nanoparticles. Gold nanoparticles may be coated, e.g., coated in a silicate and an endosomal disruptive polymer, PAsp(DET). Examples of gold nanoparticles include AURASENSE Therapeutics' Spherical Nucleic Acid (SNA™) constructs, and those described in Mout R, et al. (2017). ACS Nano 11:2452-8; Lee K, et al. (2017). Nat Biomed Eng 1:889-901.

In some embodiments, the composition further comprises iTOP. TOP refers to a combination of small molecules drives the highly efficient intracellular delivery of native proteins, independent of any transduction peptide. TOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of TOP methods and reagents include those described in D'Astolfo D S, Pagliero R J, Pras A, et al. (2015). Cell 161:674-690.

In some embodiments, the composition is in form of polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMERRNAi, VIROMERRED, VIROMER mRNA. Example methods of delivering the systems and compositions herein include those described in Bawage S S et al., Synthetic mRNA expressed CasI3a mitigates RNA virus infections, biorxiv.org/content/10.1101/370460vl.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi: 10.13140/RG.2.2.23912.16642.

In some embodiments, the composition is in form of lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In one embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

In some embodiments, the composition comprises inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

In some embodiments, the composition is in form of exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; E1-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75. In some examples, the exosome may form a complex (e.g., by binding directly or indirectly) to one or more of the various components described herein (e.g., including, but not limited to, the protein effector polypeptides (or polynucleotides encoding same), fusion proteins (or polynucleotides encoding same), guide nucleic acids (or polynucleotides encoding same), and complexes comprising same). In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Cells

Embodiments of the various aspects described herein include a cell. As used herein, the term “cell” refers to a single cell as well as to a population of (i.e., more than one) cells. A cell can be a prokaryotic or eukaryotic cell. Exemplary eukaryotic cells include a yeast cell, an insect cell, a fungal cell, a plant cell, and an animal cell (e.g., a mammalian cell).

It is noted a cell can be in vivo, in vitro or ex vivo. Accordingly, in some embodiments of any one of the aspects, the cell is in vitro. In some embodiments of any one of the aspects, the cell is ex vivo. In some embodiments of any one of the aspects, the cell is in vivo.

In some embodiments of any one of the aspects described herein, the cell is a mammalian cell. As used herein, “mammalian cell” “mammalian cell” includes cells derived from mammals, including humans, rats, mice, guinea pigs, chimpanzees, or macaques. Thus, suitable mammalian cells include, for example without limitation, human, non-human primate, cat, dog, sheep, goat, cow, horse, pig, rabbit, and rodent cells.

In some embodiments, the cell can be a primary cell derived from a subject. For example, primary cells that are derived from patients and expanded can be edited ex vivo using the pegRNA, composition, and/or prime editing systems described herein.

In some embodiments, the cell is an animal cell. For example, animal cells can be edited or re-engineered ex-vivo using the pegRNA, compositions, and/or prime editing systems described herein.to create edible tissues for food.

In some embodiments, the cell is a plant cell. For example, plant cells can be edited or re-engineered ex-vivo using the pegRNA, compositions, and/or prime editing systems described herein.to create crops that can survive better and give nutritious benefits.

In some embodiments of any one of the aspects described herein, the cell is a mismatch repair (MMR) deficient cell. As used herein, “mismatch repair deficient cell”, also referred to as “MMR deficient cell”, refers to a cell which is incapable of correcting DNA mismatches generated during DNA replication

In some embodiments of any one of the aspects described herein, the cell is a mismatch repair (MMR) competent cell. As used herein, “mismatch repair competent cell”, also referred to as “MMR competent cell”, refers to a cell which is capable of correcting DNA mismatches generated during DNA replication.

In some embodiments of any one of the aspects described herein, the cell is a modified cell. As used herein, “modified cell” refers to a recombinant (host) cell comprising at least one genetic modification that is not present in the “parent” host cell from which the modified cell is derived.

In some embodiments of any one of the aspects described herein, the cell is liver cell. Exemplary cells of the liver include but are not limited to hepatocytes, sinusoidal endothelial cells (SEC), Kupffer cells (KC), and hepatic stellate cells (HSC), as well as various immune cells In some embodiments of the any one of the aspects described herein, the cell is a hepatocyte.

In some embodiments of any one of the aspects described herein, the cell is an immune cell. An immune cell can be a cell of the lymphoid lineage. Non-limiting examples of cells of the lymphoid lineage that can be used as immune cells include T cells and Natural Killer (NK) cells. T cells express the T cell receptor (TCR), with most cells expressing α and β chains and a smaller population expressing γ and δ chains. T cells can be CD4⁺ or CD8⁺ and can include, but are not limited to, T helper cells (CD4⁺), cytotoxic T cells (also referred to as cytotoxic T lymphocytes, CTL; CD8⁺ T cells), and memory T cells, including central memory T cells, stem-cell-like memory T cells (or stem-like memory T cells), and effector memory T cells, for example, T_{EM} cells and T_{EMRA} (CD45RA⁺) cells, natural killer T cells, mucosal associated invariant T cells (MAIT), and γδ T cells. Other exemplary immune cells include, but are not limited to, macrophages, antigen presenting cells (APCs) such as dendritic cells. In some embodiments of any one of the aspects described herein, the cell is a T-cell.

In one aspect, provided herein is a cell comprising a pegRNA described herein or a nucleic acid encoding same.

In some embodiments, the cell further comprises a napDNAbp or a nucleic acid encoding same. It is noted that the cell can comprise any one of the various napDNAbp embodiments described herein.

In some embodiments, the cell further comprises a nucleic acid modifying enzyme or a nucleic acid encoding same. It is noted that the cell can comprise any one of the various nucleic acid modifying enzyme embodiments described herein.

In some embodiments of the various aspects described herein, the cell comprises a pegRNA described herein or a nucleic acid encoding same, a napDNAbp or a nucleic acid encoding same, and a nucleic acid modifying enzyme or a nucleic acid encoding same, and wherein the pegRNA is circularized, the napDNAbp is nCas9, and the nucleic acid modifying enzyme is a reverse transcriptase (e.g., MMLV RT).

In some embodiments of the various aspects described herein, the cell comprises a pegRNA described herein or a nucleic acid encoding same, a napDNAbp or a nucleic acid encoding same, and a nucleic acid modifying enzyme or a nucleic acid encoding same, and wherein the napDNAbp is attached to or tethered to the nucleic acid modifying enzyme, optionally the napDNAbp and the nucleic acid modifying enzyme are comprises in a fusion protein.

In some embodiments of the various aspects described herein, the cell comprises a pegRNA described herein or a nucleic acid encoding same, a napDNAbp or a nucleic acid encoding same, and a nucleic acid modifying enzyme or a nucleic acid encoding same, and wherein the napDNAbp is not attached to or tethered to the nucleic acid modifying enzyme.

In some embodiments of the various aspects described herein, the cell comprises a pegRNA described herein or a nucleic acid encoding same, a napDNAbp or a nucleic acid encoding same, and a nucleic acid modifying enzyme or a nucleic acid encoding same, and wherein the pegRNA does not comprise an adaptor protein recruitment domain, and optionally the pegRNA is circularized.

In another aspect provided herein is an isolated cell or progeny thereof comprising a nucleic acid modified by a pegRNA and/or method descried herein.

In yet another aspect, provided herein is a nucleic acid modified by a pegRNA and/or a method described herein.

### Kits

In another aspect provided herein is a kit comprising a pegRNA described herein or a nucleic acid encoding same. A kit is any manufacture (e.g., a package or container) comprising pegRNA described herein or a polynucleotide encoding a pegRNA described herein described herein. The manufacture can be promoted, distributed, or sold as a unit for performing the methods described herein. It is noted that the kit can comprise any one of the various pegRNA embodiments described herein.

In some embodiments, the kit further comprises a napDNAbp or a nucleic acid encoding same. It is noted that the kit can comprise any one of the various napDNAbp embodiments described herein.

In some embodiments, the kit further comprises a nucleic acid modifying enzyme or a nucleic acid encoding same. It is noted that the kit can comprise any one of the various nucleic acid modifying enzyme embodiments described herein.

In some embodiments of the various aspects described herein, the kit comprises a pegRNA described herein or a nucleic acid encoding same, a napDNAbp or a nucleic acid encoding same, and a nucleic acid modifying enzyme or a nucleic acid encoding same, and wherein the pegRNA is circularized, the napDNAbp is nCas9, and the nucleic acid modifying enzyme is a reverse transcriptase (e.g., MMLV RT).

In some embodiments of the various aspects described herein, the kit comprises a pegRNA described herein or a nucleic acid encoding same, a napDNAbp or a nucleic acid encoding same, and a nucleic acid modifying enzyme or a nucleic acid encoding same, and wherein the napDNAbp is attached to or tethered to the nucleic acid modifying enzyme, optionally the napDNAbp and the nucleic acid modifying enzyme are comprises in a fusion protein.

In some embodiments of the various aspects described herein, the kit comprises a pegRNA described herein or a nucleic acid encoding same, a napDNAbp or a nucleic acid encoding same, and a nucleic acid modifying enzyme or a nucleic acid encoding same, and wherein the napDNAbp is not attached to or tethered to the nucleic acid modifying enzyme.

In some embodiments of the various aspects described herein, the kit comprises a pegRNA described herein or a nucleic acid encoding same, a napDNAbp or a nucleic acid encoding same, and a nucleic acid modifying enzyme or a nucleic acid encoding same, and wherein the pegRNA doesnot comprise an adaptor protein recruitment domain, and optionally the pegRNA is circularized.

In some embodiments of the various aspects described herein, the kit further comprises a nucleic acid delivery system.

The kits described herein can optionally comprise additional components and reagents. As will be appreciated by one of skill in the art, components of the kit can be provided in any desired form, e.g., in a lyophilized form, a liquid form, a solid form, or a concentrated. In some embodiments of the various aspects described herein, the kit can comprise ampoules, syringes, or the like.

In some embodiments, the kit can comprise informational material. The informational material can be descriptive, instructional, marketing or other material that relates to the methods described herein. The informational material of the kits is not limited in its form. In some embodiments, the informational material can include information about production of the components, concentration, date of expiration, batch or production site information, and so forth. In one embodiment, the informational material relates to methods for using or administering the components of the kit.

It is notes that the components of a kit can provided singularly or in any combination as a kit. Such a kit includes the components described herein and packaging materials thereof.

In some embodiments, the components in a kit can be provided in a watertight or gas tight container which in some embodiments is substantially free of other components of the kit. For example, the components of the kit can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the reagents can be provided in sterile form and should not contain microorganisms or other contaminants. When the components described herein are provided in a liquid solution, the liquid solution preferably is an aqueous solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### One or More Nucleotide Changes

As used herein, “one or more nucleotide changes” refers to an alternation in one or more nucleotides in a target sequence, such as in DNA or RNA. One or more nucleotide changes can include insertions of one or more nucleotides, substitutions of one or more nucleotides, deletions of one or more nucleotides, or a combination of any such nucleotide changes, as compared to the target sequence, e.g., as compared to the double-stranded target DNA sequence.

In some embodiments of any one of the aspects described herein, the one or more nucleotide changes comprises a transition from one nucleotide to another nucleotide. As used herein, “transition” refer to the interchange of purine nucleobases (A↔G) or the interchange of pyrimidine nucleobases (C↔T). This class of interchanges involves nucleobases of similar shape. The pegRNAs. compositions and methods disclosed herein are capable of inducing one or more transitions in a target nucleic acid, e.g., DNA molecule. The pegRNA, compositions and methods disclosed herein are also capable of inducing both transitions and transversion in the same target nucleic acid, e.g., DNA molecule. These changes involve A↔G, G↔A, C↔T, or T↔C. In the context of a double-strand nucleic acid with Watson-Crick paired nucleobases, transitions refer to the following base pair exchanges: A:T↔G:C, G:G↔A:T, C:G↔T:A, or T:A↔C:G. Thus, in some embodiments, the one or more nucleotide changes comprises a transition selected from the group consisting of: (a) T to C; (b) A to G; (c) C to T; (d) G to A; and (e) A to I.

In some embodiments of any one of the aspects described herein, the one or more nucleotide changes comprises a transversion. As used herein, “transversion” refer to the interchange of purine nucleobases for pyrimidine nucleobases, or in the reverse and thus, involve the interchange of nucleobases with dissimilar shape. These changes involve T↔A, T4↔G, C↔G, C↔A, A↔T, A↔C, G↔C, and G↔T. In the context of a double-strand nucleic acid with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target nucleic acid, e.g., DNA molecule. Thus, in some embodiments of any one of the aspects described herein, the one or more nucleotide changes comprises a transversion selected from the group consisting of: (a) T to A; (b) T to G; (c) C to G; (d) C to A; (e) A to T; (f) A to C; (g) G to C; (h) G to T; and (i) and A to I.

In some embodiments of any of the aspects, the one or more nucleotide changes comprises a nucleotide insertion. As used herein, the term “nucleotide insertion” refers to the insertion of one or more additional nucleotides into a predetermined or native nucleotide sequence. Thus, in some embodiments, the one or more nucleotide changes comprises an insertion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides. For example, the one or more nucleotide changes comprises deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides.

In some embodiments of any of the aspects, the one or more nucleotide changes comprises a nucleotide deletion. As used herein, the term “nucleotide deletion” refers to the deletion of one or more nucleotides into a predetermined or native nucleotide sequence. Thus, in some embodiments, the one or more nucleotide changes comprises deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides. For example, the one or more nucleotide changes comprises deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides.

The position of the desired one or more nucleotide changes, e.g., edit can be in any position following downstream of the nick site on the PAM strand, which can include position +1, +2, +3, +4, +, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, +55, +56, +57, +58, +59, +60, +61, +62, +63, +64, +65, +66, +67, +68, +69, +70, +71, +72, +73, +74, +75, +76, +77, +78, +79, +80, +81, +82, +83, +84, +85, +86, +87, +88, +89, +90, +91, +92, +93, +94, +95, +96, +97, +98, +99, +100, +101, +102, +103, +104, +105, +106, +107, +108, +109, +110, +111, +112, +113, +114, +115, +116, +117, +118, +119, +120, +121, +122, +123, +124, +125, +126, +127, +128, +129, +130, +131, +132, +133, +134, +135, +136, +137, +138, +139, +140, +141, +142, +143, +144, +145, +146, +147, +148, +149, or +150, or more (relative to the downstream position of a nick site). Once the 3′ end single stranded DNA (containing the edit of interest) replaces the endogenous 5′ end single stranded DNA, the DNA repair and replication processes will result in permanent installation of the edit site on the PAM strand, and then correction of the mismatch on the non-PAM strand that will exist at the edit site. In this way, the edit will extend to both strands of DNA on the target DNA site. It will be appreciated that reference to “edited strand” and “non-edited” strand only intends to delineate the strands of DNA involved in the PE mechanism. The “edited strand” is the strand that first becomes edited by replacement of the 5′ ended single strand DNA immediately downstream of the nick site with the synthesized 3′ ended single stranded DNA containing the desired edit. The “non-edited” strand is the strand pair with the edited strand, but which itself also becomes edited through repair and/or replication to be complementary to the edited strand, and in particular, the edit of interest.

used herein, the term “edit” “editing” or “edited” refers to a method of altering a nucleic acid sequence of a polynucleotide (e.g., for example, a wild type naturally occurring nucleic acid sequence or a mutated naturally occurring sequence) by selective transition, transversion, insertion and/or deletion of one or more nucleotides in a specific target nucleic acid, such as a genomic target. Exemplary genomic targets include, but are not limited to, a chromosomal region, a gene, a promoter, an open reading frame or any nucleic acid sequence.

In some embodiments of any of the aspects, the primer binding site comprises a sequence having 100% complementarity to a region upstream of the nick site in the second strand of the double-stranded target nucleic acid.

### Methods

In another aspect, provided herein is a method of introducing one or more changes in the nucleotide sequence of a target nucleic acid. The method comprises contacting a double-stranded target nucleic acid (e.g., DNA) with a prime editing system described herein.

In some embodiments of any one of the aspects, the target nucleic acid can be in a cell.

It is noted that when the target nucleic acid is in a cell, the method comprises administering the prime editing system or components thereof to the cell. Methods for administering a prime editing system or components thereof to a cell are well known and available to one of skill in the art. As used herein, administering the prime editing system or components thereof to the cell means contacting the cell with the prime editing system or components thereof so that the prime editing system or components thereof are taken up by the cell. Generally, the cell can be contacted with the prime editing system or components thereof in a cell culture e.g., in vitro or ex vivo, or the prime editing system or components thereof can be administrated to a subject, e.g., in vivo. The term “contacting” or “contact” as used herein in connection with contacting a cell includes subjecting the cells to an appropriate culture media, which comprises the prime editing system or components thereof. Where the cell is in vivo, “contacting” or “contact” includes administering the prime editing system or components thereof, e.g., in a pharmaceutical composition to a subject via an appropriate administration route such that the prime editing system or components thereof contacts the cell in vivo.

For example, when the cell is in vitro, said administering to the cell can include subjecting the cell to an appropriate culture media which comprises the prime editing system or components thereof. Where the cell is in vivo, said administering to the cell includes administering the prime editing system or components thereof to a subject via an appropriate administration route such that the compound is administered to the cell in vivo.

The cell to be administered can be any desired cell. For example, the cell comprising the target nucleic acid can be mammalian cell. In some embodiments, the cell comprising the target nucleic acid can be a human cell. In some embodiments of any of the aspects, the cell comprising the target nucleic acid is a mismatch repair (MMR) deficient cell. In some other embodiments, the cell comprising the target nucleic acid is a mismatch repair (MMR) competent cell. In some embodiments of any of the aspects, the cell comprising the target nucleic acid is an immune cell, e.g., a T-cell. In some embodiments of any of the aspects, the cell comprising the target nucleic acid is a liver cell, e.g., a hepatocyte.

In some embodiments, the cell is selected from the group consisting of hematopoietic stem cells; T cells; liver cells (hepatocytes); pancreatic islet beta cells; and lung epithelial cells.

In some embodiments, the method is a method of therapeutic genome editing. For example, the method comprises administering to a target cell selected from the group consisting of: (a) hematopoietic stem cells; (b) T cells; (c) liver cells (hepatocytes); (d) pancreatic islet beta cells; (e) lung epithelial cells.

### “Disease-Associated” Genes

In some embodiments of any of the aspects, the one or more changes in the nucleotide sequence comprises a correction to a disease-associated gene. As used herein, a “disease-associated” gene or polynucleotide refers to any gene or polynucleotide which is yielding transcription or translation products at an abnormal level or in an abnormal form in cells derived from a disease-affected tissues compared with tissues or cells of anon disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web, contents of all which are incorporated herein by reference in their entireties.

For example, the disease associated gene is associated with disorder selected from the group consisting of: **** 22q13.3 deletion syndrome, 2-methyl-3-hydroxybutyric aciduria, 3 beta-Hydroxysteroid dehydrogenase deficiency, 3 Methylcrotonyl-CoA carboxylase 1 deficiency, 3-methylcrotonyl CoA carboxylase 2 deficiency, 3-Methylglutaconic aciduria type 1, 3-Methylglutaconic aciduria type 2, 3-Methylglutaconic aciduria type 3, Optic atrophy and cataract, autosomal dominant, 3-methylglutaconic aciduria type V, 3-methylglutaconic aciduria with cataracts, neurologic involvement, and neutropenia, 3-methylglutaconic aciduria with deafness, encephalopathy, and Leigh-like syndrome, 3-methylglutaconic aciduria, type VIII, 3-Oxo-5 alpha-steroid delta 4-dehydrogenase deficiency, 46,XY sex reversal, type 3, Premature ovarian failure 7, 46,XY sex reversal, type 5, 46,XY sex reversal, type 6, Inborn genetic diseases, ABCA4-Related Disorders, Age-related macular degeneration 2, Cone-rod dystrophy 3, MACULAR DEGENERATION, AGE-RELATED, 2, SUSCEPTIBILITY TO, Mandibulofacial dysostosis with mental deficiency, Retinitis pigmentosa 19, Stargardt disease, Stargardt disease 1, Abdominal obesity-metabolic syndrome 3, Abdominal pain, Fever, Mood changes, Visual loss, Vomiting, Abetalipoproteinaemia, Abnormal bleeding, von Willebrand disease type 3, von Willebrand disorder, von Willebrand disease type 2, Abnormal blistering of the skin, Abnormal urinary color, Constipation, Migraine, Charcot-Marie-Tooth disease, type 2, Charcot-Marie-Tooth disease, type 2A2A, Hereditary motor and sensory neuropathy with optic atrophy, Abnormal cortical gyration, FACIAL DYSMORPHISM, HYPERTRICHOSIS, EPILEPSY, INTELLECTUAL/DEVELOPMENTAL DELAY, AND GINGIVAL OVERGROWTH SYNDROME, Generalized hypertrichosis, Gingival overgrowth, Intellectual disability, Seizures, Abnormal macular morphology, Blindness, Pigmentary retinopathy, Rare genetic deafness, Retinal pigment epithelial atrophy, Retinitis pigmentosa 39, Rod-cone dystrophy, Usher syndrome, type 2A, Albinism, Fair hair, Horizontal nystagmus, Hypopigmentation of the skin, Ocular albinism, Strabismus, Tyrosinase-negative oculocutaneous albinism, Abnormality of brain morphology, Combined oxidative phosphorylation deficiency 7, Spastic paraplegia 55, autosomal recessive, Abnormality of cardiovascular system morphology, Congenital diaphragmatic hernia, Pancreatic agenesis and congenital heart disease, Juvenile myelomonocytic leukemia, Noonan syndrome, Noonan syndrome 1, Rasopathy, Abnormality of coagulation, Hyperammonemia, Ornithine carbamoyltransferase deficiency, Protein avoidance, Abnormality of retinal pigmentation, Cataract, Exudative retinopathy, Nystagmus, Optic disc drusen, Progressive visual loss, Retinal detachment, Retinal exudate, Rod-cone dystrophy, Unilateral strabismus, CONGENITAL HEART DEFECTS AND SKELETAL MALFORMATIONS SYNDROME, Failure to thrive, Abnormality of the anterior fontanelle, Abnormality of the cerebral white matter, Central hypotonia, Cryptorchidism, Deep plantar creases, Global developmental delay, Macrocephalus, Carious teeth, Intellectual disability, Microcephaly, Muscular hypotonia, Oral-pharyngeal dysphagia, Skeletal muscle atrophy, Cohen syndrome, Retinal dystrophy, CNS demyelination, Cerebral cortical atrophy, Difficulty walking, Dysmetria, Gait ataxia, Gait imbalance, Gout, Hypertension, Impaired vibration sensation in the lower limbs, Leukoencephalopathy with Brainstem and Spinal Cord Involvement and Lactate Elevation, Sensorimotor neuropathy, Talipes equinovarus, Aplasia of the ovary, Aplastic anemia, Atrial septal defect, Dilation of lateral ventricles, Downslanted palpebral fissures, Dry skin, Erythroid hypoplasia, Hemangioma, Hepatomegaly, Hypotelorism, Intracerebral periventricular calcifications, Low-set, posteriorly rotated ears, Pulmonary arterial hypertension, Relative macrocephaly, Vaginal hydrocele, Wide anterior fontanel, External ophthalmoplegia, OPHTHALMOPLEGIA, EXTERNAL, WITH RIB AND VERTEBRAL ANOMALIES, Scoliosis, Anonychia, Dominant dystrophic epidermolysis bullosa with absence of skin, Dystrophic epidermolysis bullosa, Epidermolysis bullosa dystrophica inversa, autosomal recessive, Epidermolysis bullosa dystrophica, AD, Epidermolysis bullosa dystrophica, AR, Epidermolysis bullosa pruriginosa, Generalized dominant dystrophic epidermolysis bullosa, Nail disorder, nonsyndromic congenital, 8, Nail dystrophy, Pretibial epidermolysis bullosa, Recessive dystrophic epidermolysis bullosa, Skin erosion, Transient bullous dermolysis of the newborn, Ptosis, Pulmonic stenosis, Short stature, Autistic disorder of childhood onset, Delayed speech and language development, Iris coloboma, Motor delay, Dominant hereditary optic atrophy, Mitochondrial diseases, Achondrogenesis, type IB, Atelosteogenesis type 2, Diastrophic dysplasia, Multiple epiphyseal dysplasia 4, Osteochondrodysplasia, SLC26A2-Related Disorders, Achondrogenesis, type II, Avascular necrosis of the head of femur, Coxa plana, Czech dysplasia metatarsal type, Epiphyseal dysplasia, multiple, with myopia and conductive deafness, Kniest dysplasia, Osteoarthritis with mild chondrodysplasia, Platyspondylic lethal skeletal dysplasia Torrance type, Spondyloepiphyseal dysplasia, Spondyloepiphyseal dysplasia, stanescu type, Spondylometaphyseal dysplasia, Spondyloperipheral dysplasia, Stickler syndrome type 1, Stickler syndrome, type I, nonsyndromic ocular, Achromatopsia, Achromatopsia 2, Achromatopsia 3, Achromatopsia 5, Achromatopsia 7, Acid-labile subunit deficiency, Acne inversa, familial, 2, Acne inversa, familial, 3, Alzheimer disease, type 3, Frontotemporal dementia, Pick's disease, Acrocephalosyndactyly type I, Antley-Bixler syndrome without genital anomalies or disordered steroidogenesis, Bent bone dysplasia syndrome, Craniosynostosis, Craniosynostosis, nonclassifiable autosomal dominant, Crouzon syndrome, Cutis Gyrata syndrome of Beare and Stevenson, FGFR2 related craniosynostosis, Jackson-Weiss syndrome, Levy-Hollister syndrome, Neoplasm of stomach, Pfeiffer syndrome, Saethre-Chotzen syndrome, Scaphocephaly, maxillary retrusion, and mental retardation, Acrodysostosis 2, with or without hormone resistance, Acromicric dysplasia, Cardiovascular phenotype, Ectopia lentis, isolated, autosomal dominant, Geleophysic dysplasia 2, MASS syndrome, Marfan Syndrome/Loeys-Dietz Syndrome/Familial Thoracic Aortic Aneurysms and Dissections, Marfan lipodystrophy syndrome, Marfan syndrome, Stiff skin syndrome, Thoracic aortic aneurysm and aortic dissection, Weill-Marchesani syndrome 2, ACTH resistance, Glucocorticoid Deficiency, Acute aortic dissection, Aortic aneurysm, familial thoracic 10, Familial thoracic aortic aneurysm, Acute intermittent porphyria, Thrombocytopenia, Acute myeloid leukemia, Beckwith-Wiedemann syndrome, Sotos syndrome 1, Brainstem glioma, Hepatocellular carcinoma, Neoplasm of brain, Neoplasm of the large intestine, D-2-hydroxyglutaric aciduria 2, Multiple myeloma, Myelodysplastic syndrome, Myelodysplastic syndrome progressed to acute myeloid leukemia, Myeloproliferative/lymphoproliferative neoplasms, familial (multiple types), susceptibility to, Spinocerebellar ataxia 35, Acute neuronopathic Gaucher's disease, Gaucher disease, Gaucher disease type 3C, Gaucher disease, perinatal lethal, Gaucher's disease, type 1, Lewy body dementia, Parkinson disease, late-onset, Subacute neuronopathic Gaucher's disease, Adams-Oliver syndrome 4, Adams-Oliver syndrome 5, Adams-Oliver syndrome 6, Addison's disease, Adrenoleukodystrophy, Adenine phosphoribosyltransferase deficiency, Adenocarcinoma of prostate, Adenocarcinoma of stomach, Carcinoma of colon, Chronic lymphocytic leukemia, Cutaneous melanoma, Follicular thyroid carcinoma, Lung adenocarcinoma, Malignant melanoma of skin, Malignant neoplasm of body of uterus, Neoplasm of the thyroid gland, Non-small cell lung cancer, Carcinoma of esophagus, Medulloblastoma, Pancreatic adenocarcinoma, Transitional cell carcinoma of the bladder, Cardio-facio-cutaneous syndrome, Cardiofaciocutaneous syndrome 4, Cardiomyopathy, dilated, 1NN, LEOPARD syndrome 2, Noonan syndrome 5, Noonan syndrome with multiple lentigines, Rasopathy, Adenylate kinase deficiency, hemolytic anemia due to, Adenylosuccinate lyase deficiency, Difficulty standing, Generalized myoclonic seizures, Inability to walk, Progressive neurologic deterioration, Severe global developmental delay, Adermatoglyphia, Keratoderma with scleroatrophy of the extremities, Adolescent nephronophthisis, Meckel syndrome type 7, Polycystic kidney dysplasia, Renal-hepatic-pancreatic dysplasia, Adrenal insufficiency, congenital, with 46,XY sex reversal, partial or complete, Adrenocorticotropic hormone deficiency, Adult hypophosphatasia, Childhood hypophosphatasia, Hypophosphatasia, Infantile hypophosphatasia, Adult junctional epidermolysis bullosa, Amelogenesis imperfecta, type IA, Junctional epidermolysis bullosa gravis of Herlitz, Adult neuronal ceroid lipofuscinosis, GBE1-Related Disorders, Glycogen storage disease IV, classic hepatic, Glycogen storage disease IV, nonprogressive hepatic, Glycogen storage disease, type IV, Polyglucosan body disease, adult, ADULT syndrome, Advanced sleep phase syndrome, familial, 3, Afibrinogenemia, congenital, Hypodysfibrinogenemia, congenital, Hypofibrinogenemia, Agammaglobulinemia 7, autosomal recessive, Immunodeficiency 36, SHORT syndrome, Agammaglobulinemia, non-Bruton type, X-linked agammaglobulinemia, Agenesis of corpus callosum, Cerebellar vermis hypoplasia, Intellectual developmental disorder with persistence of fetal hemoglobin, Aplastic anemia, susceptibility to, Deeply set eye, Microcephaly, Short stature, Shwachman syndrome, Splenomegaly, Age-related macular degeneration 1, Bull's eye maculopathy, Retinal dystrophy, Retinitis pigmentosa, Retinal dystrophy, early-onset severe, Aicardi Goutieres syndrome 2, Aicardi Goutieres syndrome 3, Aicardi Goutieres syndrome 4, Aicardi Goutieres syndrome 5, Singleton-Merten syndrome 1, AL KAISSI SYNDROME, Alacrima, achalasia, and mental retardation syndrome, Alagille syndrome 1, Deafness, congenital heart defects, and posterior embryotoxon, Tetralogy of Fallot, Alagille syndrome 2, Alazami syndrome, Albinism, ocular, with sensorineural deafness, Nonsyndromic Oculocutaneous Albinism, Oculocutaneous albinism type 1B, Skin/hair/eye pigmentation, variation in, 3, Aldosterone Producing Adrenal Cortex Adenoma, Alexander Disease, ALG12-congenital disorder of glycosylation, ALG9 congenital disorder of glycosylation, Alkaptonuria, Allan-Herndon-Dudley syndrome, Intellectual disability, Alpha Thalassemia, Alpha-1-antitrypsin deficiency, Chronic obstructive pulmonary disease, PI S, Hemorrhagic disease due to alpha-1-antitrypsin Pittsburgh mutation, PI NULL(BELLINGHAM), PI Q0(BELLINGHAM), PI NULL(LUDWIGSHAFEN), PI Q0(LUDWIGSHAFEN), PI S(IIYAMA), Alpha-B crystallinopathy, Dilated cardiomyopathy 111, Alpha-methylacyl-CoA racemase deficiency, Bile acid synthesis defect, congenital, 4, Alpha-thalassemia, Hmong type, Alport syndrome, Alport syndrome 1, X-linked recessive, Alport syndrome 3, autosomal dominant, Alport syndrome, autosomal recessive, Benign familial hematuria, Alstrom syndrome, Alternating hemiplegia of childhood 2, Cerebellar ataxia, areflexia, pes cavus, optic atrophy and sensorinural hearing loss, Dystonia 12, Global developmental delay, Hemiplegia, Oculogyric crisis, Alzheimer disease, familial, 3, with spastic paraparesis and apraxia, Alzheimer disease, familial, 3, with unusual plaques, Alzheimer disease, familial, with spastic paraparesis and unusual plaques, Alzheimer disease, type 1, Alzheimer's disease, Cerebral amyloid angiopathy, APP-related, Alzheimer disease, type 4, Amelogenesis imperfecta, hypocalcification type, Amelogenesis imperfecta, hypomaturation type IIA4, Amelogenesis imperfecta, hypomaturation type IIA5, Amelogenesis imperfecta, hypomaturation type, IIA1, Amelogenesis imperfecta, type 1E, Amyloidogenic transthyretin amyloidosis, Cardiomyopathy, Carpal tunnel syndrome, Dystransthyretinemic euthyroidal hyperthyroxinemia, Amyotrophic lateral sclerosis 14 without frontotemporal dementia, Amyotrophic lateral sclerosis 14, with or without frontotemporal dementia, Inclusion body myopathy with early-onset paget disease and frontotemporal dementia, Amyotrophic lateral sclerosis 16, juvenile, Distal spinal muscular atrophy, autosomal recessive 2, Amyotrophic lateral sclerosis 17, Frontotemporal Dementia, Chromosome 3-Linked, Amyotrophic lateral sclerosis 18, Amyotrophic lateral sclerosis 21, Amyotrophic lateral sclerosis 22 with or without frontotemporal dementia, AMYOTROPHIC LATERAL SCLEROSIS 23, Amyotrophic lateral sclerosis type 1, Amyotrophic lateral sclerosis type 10, TARDBP-related frontotemporal dementia, Amyotrophic lateral sclerosis type 11, Charcot-Marie-Tooth disease, Charcot-Marie-Tooth disease type 4, Charcot-Marie-Tooth disease, type 4J, Amyotrophic lateral sclerosis type 2, Infantile-onset ascending hereditary spastic paralysis, Juvenile primary lateral sclerosis, Amyotrophic lateral sclerosis type 4, Distal spinal muscular atrophy, Spinocerebellar ataxia autosomal recessive 1, Amyotrophic lateral sclerosis type 5, Charcot-Marie-Tooth disease, axonal type 2X, Spastic paraplegia 11, autosomal recessive, Amyotrophic lateral sclerosis type 6, Tremor, hereditary essential, 4, Amyotrophic lateral sclerosis type 8, Amyotrophic lateral sclerosis, typical, Spinal muscular atrophy, late-onset, finkel type, Amyotrophic lateral sclerosis type 9, Amyotrophy, hereditary neuralgic, Anauxetic dysplasia 2, Anauxetic dysplasia, Metaphyseal chondrodysplasia, McKusick type, Metaphyseal dysplasia without hypotrichosis, Andermann syndrome, Andersen Tawil syndrome, Atrial fibrillation, familial, 9, Congenital long QT syndrome, Short QT syndrome 3, Arrhythmia, Supraventricular tachycardia, Ventricular tachycardia, Androgen resistance syndrome, Bulbo-spinal atrophy X-linked, Anemia sideroblastic and spinocerebellar ataxia, Anemia without thromobocytopenia, X-linked, GATA-1-related thrombocytopenia with dyserythropoiesis, ANEMIA, CONGENITAL DYSERYTHROPOIETIC, TYPE Ib, Anemia, nonspherocytic hemolytic, due to G6PD deficiency, Susceptibility to malaria, FG syndrome 4, Mental retardation and microcephaly with pontine and cerebellar hypoplasia, Anemia, sideroblastic, 4, Anemia, sideroblastic, pyridoxine-refractory, autosomal recessive, Anemia, Beta-plus-thalassemia, HEMOGLOBIN T (CAMBODIA), Hb SS disease, Hemoglobin E, Hemoglobin E disease, Hemoglobin E/beta thalassemia disease, Malaria, resistance to, Heinz body anemia, Splenomegaly, Angelman syndrome, Angelman syndrome-like, Atypical Rett syndrome, Early infantile epileptic encephalopathy 2, Angioedema, Angiopathy, hereditary, with nephropathy, aneurysms, and muscle cramps, Aniridia 1, Anomalous origin of coronary artery from the pulmonary artery, Ciliary dyskinesia, primary, 27, Clinodactyly of the 5th finger, Cough, Anonychia, Anosmia, Arhinia choanal atresia microphthalmia, Anterior segment dysgenesis 3, ANTERIOR SEGMENT DYSGENESIS 4, PETERS ANOMALY SUBTYPE, Anterior Segment Dysgenesis 5—multiple subtypes, foveal hypoplasia 1 with or without anterior segment anomalies, anterior segment dysgenesis 7, anterior segment dysgenesis 8, Antley-Bixler syndrome with genital anomalies and disordered steroidogenesis, Disordered steroidogenesis due to cytochrome p450 oxidoreductase deficiency, FGFR2 related craniosynostosis, Pfeiffer syndrome, Pfeiffer syndrome—type III, Aortic aneurysm, familial thoracic 4, Aortic aneurysm, familial thoracic 4, Aortic aneurysm, familial thoracic 6; Aortic aneurysm, familial thoracic 6, Cardiovascular phenotype, Connective tissue disorder, Moyamoya disease 5, Multisystemic smooth muscle dysfunction syndrome, Multisystemic smooth muscle dysfunction syndrome, Thoracic aortic aneurysm and aortic dissection, alterations of great arteries and veins, Thoracic aortic aneurysm and aortic dissection, Aortic root dilatation, Arachnodactyly, Dissecting aortic dilatation, Ectopia lentis, Ischemic stroke, Marfan Syndrome/Loeys-Dietz Syndrome/Familial Thoracic Aortic Aneurysms and Dissections, Marfan syndrome, Melanoma, Severe Myopia, Thoracic aortic aneurysm and aortic dissection, not provided, Aplasia/Hypoplasia of the corpus callosum, Dyskinesia, Dystonia, Global brain atrophy, Intellectual disability, severe, Motor delay, Progressive microcephaly, Rolandic epilepsy, Severe global developmental delay, Unverricht-Lundborg syndrome, Aplastic anemia, Pulmonary fibrosis and/or bone marrow failure, telomere-related, 1, Pulmonary fibrosis and/or bone marrow failure, telomere-related, 2, Shwachman syndrome, APOE4(+), APOLIPOPROTEIN A-I (GIESSEN), APOLIPOPROTEIN A-I (MARBURG), APOLIPOPROTEIN A-I (MUNSTER3B), APOLIPOPROTEIN A-I (MUNSTER3C), Apolipoprotein A-I deficiency, APOLIPOPROTEIN C-II (AFRICAN), Apolipoprotein c-ii variant, Apparent mineralocorticoid excess, APRT deficiency, Japanese type, Adenine phosphoribosyltransferase deficiency, Arginase deficiency, Arginine:glycine amidinotransferase deficiency, Argininosuccinate lyase deficiency, Arhinia choanal atresia microphthalmia, Facioscapulohumeral muscular dystrophy 2, ARMC9-related Joubert syndrome, JOUBERT SYNDROME 30, Aromatase deficiency, Arrhythmia, Contractures of the joints of the lower limbs, Elbow flexion contracture, Limb-girdle muscular dystrophy, type 2A, Muscle weakness, Muscular dystrophy, Arrhythmogenic right ventricular cardiomyopathy, Arrhythmogenic right ventricular cardiomyopathy, type 10, Arrhythmogenic right ventricular cardiomyopathy, type 11, Dilated cardiomyopathy with woolly hair and keratoderma, Epidermolysis bullosa, lethal acantholytic, Keratosis palmoplantaris striata II, Skin fragility woolly hair syndrome, Primary dilated cardiomyopathy, Cardiomyopathy, dilated, with woolly hair, keratoderma, and tooth agenesis, Cardiomyopathy, Arrhythmogenic right ventricular dysplasia, familial, 11, with mild palmoplantar keratoderma and woolly hair, Arrhythmogenic right ventricular dysplasia, familial, 13, Arrhythmogenic right ventricular dysplasia, familial, 2, Cardiovascular phenotype, Catecholaminergic polymorphic ventricular tachycardia, Long QT syndrome, Ventricular fibrillation, Arterial calcification of infancy, Arthrogryposis multiplex congenita, Lethal congenital contracture syndrome 9, Arthrogryposis renal dysfunction cholestasis syndrome, ARTHROGRYPOSIS, CLEFT PALATE, CRANIOSYNOSTOSIS, AND IMPAIRED INTELLECTUAL DEVELOPMENT, ARTHROGRYPOSIS, DISTAL, TYPE 2B3, ARTHROGRYPOSIS, DISTAL, TYPE 2B4, Aspartylglucosaminuria, Aspartylglucosaminuria, finnish type, Asphyxiating thoracic dystrophy 4, Finnish congenital nephrotic syndrome, Jeune thoracic dystrophy, Nephronophthisis, Nephronophthisis 12, Type IV short rib polydactyly syndrome, Asplenia, isolated congenital, Astigmatism, Cryptorchidism, Epicanthus, Esotropia, Global developmental delay, Hypermetropia, Hypertelorism, Intellectual disability, Retrognathia, Wide nasal bridge, Astrocytoma, Brainstem glioma, Juvenile myelomonocytic leukemia, Multiple myeloma, Neoplasm of the large intestine, Neuroblastoma, Noonan syndrome, Rasopathy, Squamous cell lung carcinoma, Asymmetric septal hypertrophy, Dyspnea, Familial hypertrophic cardiomyopathy 4, Heart block, Hypertrophic cardiomyopathy, Left ventricular noncompaction 10, Noncompaction cardiomyopathy, Primary familial hypertrophic cardiomyopathy, Tachycardia, Ventricular extrasystoles, Ataxia and retinitis pigmentosa with isolated vitamin E deficiency, Ataxia with vitamin E deficiency, Ataxia, Friedreich-like, with isolated vitamin E deficiency, Ataxia, spastic, 1, autosomal dominant, Spastic paraplegia, Expressive language delay, Hypotonia, ataxia, and delayed development syndrome, Muscular hypotonia, Ataxia-oculomotor apraxia type 1, Early infantile epileptic encephalopathy 10, Ateleiotic dwarfism, Short stature with microcephaly and distinctive facies, Short stature, microcephaly, and endocrine dysfunction, Atelosteogenesis type 1, Atrial septal defect 7 with or without atrioventricular conduction defects, Atrial septal defect, Noncompaction cardiomyopathy, Ventricular fibrillation, Atrophia bulborum hereditaria, Exudative vitreoretinopathy 5, Persistent hyperplastic primary vitreous, autosomal recessive, Atrophoderma vermiculatum, Keratosis pilaris, ATR-X syndrome, Acquired hemoglobin H disease, Inborn genetic diseases, Mental retardation-hypotonic facies syndrome X-linked, 1, Attention deficit hyperactivity disorder, Hearing impairment, Obsessive-compulsive behavior, Seizures, Atypical hemolytic uremic syndrome, Hemolytic uremic syndrome, atypical, susceptibility to, 7, Nephrotic syndrome, type 7, Atypical hemolytic-uremic syndrome 1, Atypical hemolytic-uremic syndrome 2, Atypical mycobacteriosis, familial, X-linked 2, Chronic granulomatous disease, Atypical Rett syndrome, Early infantile epileptic encephalopathy 2, Auditory neuropathy, autosomal recessive, 1, Deafness, autosomal recessive 9, AU-KLINE SYNDROME, Auriculocondylar syndrome 1, Auriculocondylar syndrome 2, Autoimmune lymphoproliferative syndrome, type 1a, Autoimmune lymphoproliferative syndrome, type V, Autoimmune polyglandular syndrome type 1, autosomal dominant, Autoimmune polyglandular syndrome type 1, with reversible metaphyseal dysplasia, Polyglandular autoimmune syndrome, type 1, Autoinflammation with infantile enterocolitis, Syndrome of entercolitis and autoinfimmation caused by mutation of NLRC4 (SCAN4), Autoinflammation, panniculitis, and dermatosis syndrome, Autoinflammatory syndrome, familial, Behcet-like, Autonomic nervous system dysfunction, Autosomal dominant distal hereditary motor neuropathy, Charcot-Marie-Tooth disease type 2C, Distal spinal muscular atrophy, congenital nonprogressive, Neuromuscular Diseases, Skeletal dysplasia, Charcot-Marie-Tooth disease, axonal, type 2S, Inborn genetic diseases, Spinal muscular atrophy, distal, autosomal recessive, 1, Scapuloperoneal spinal muscular atrophy, Spinal muscular atrophy, lower extremity-predominant, 2A, autosomal dominant, Autosomal dominant hypohidrotic ectodermal dysplasia, Autosomal recessive hypohidrotic ectodermal dysplasia syndrome, Autosomal dominant intermediate Charcot-Marie-Tooth disease, Congenital myotonia, autosomal dominant form, Congenital myotonia, autosomal recessive form, Myotonia congenita, Autosomal dominant isolated somatotropin deficiency, Autosomal dominant nocturnal frontal lobe epilepsy, Epilepsy, nocturnal frontal lobe, type 1, Autosomal dominant optic atrophy plus syndrome, Mitochondrial diseases, Autosomal dominant progressive external ophthalmoplegia with mitochondrial DNA deletions 1, Cerebellar ataxia infantile with progressive external ophthalmoplegia, Mitochondrial DNA depletion syndrome 1 (MNGIE type), Mitochondrial DNA depletion syndrome 4B, MNGIE type, Progressive sclerosing poliodystrophy, Sensory ataxic neuropathy, dysarthria, and ophthalmoparesis, Autosomal recessive congenital ichthyosis 1, Ichthyosis, Autosomal recessive congenital ichthyosis 4B, Autosomal recessive congenital ichthyosis 5, Autosomal recessive congenital ichthyosis 6, Autosomal recessive congenital ichthyosis 3, Autosomal recessive cutis laxa type 1B, Autosomal recessive cutis laxa type IA, Autosomal recessive Dejerine-Sottas syndrome, Charcot-Marie-Tooth disease type 4, Charcot-Marie-Tooth disease, demyelinating, type 4F, Autosomal recessive hearing impairment with normal menstrual cycles, Perrault syndrome 3, Autosomal recessive hypophosphatemic bone disease, Autosomal recessive hypophosphatemic vitamin D refractory rickets, Autosomal Recessive Hypotrichosis with Woolly Hair, Autosomal recessive woolly hair 3, Autosomal recessive non-syndromic intellectual disability, Autosomal recessive non-syndromic sensorineural deafness type DFNB, Autosomal recessive polycystic kidney disease, Autosomal recessive severe congenital neutropenia, Specific granule deficiency, Specific granule deficiency 2, Autosomal recessive spastic ataxia, Spastic ataxia Charlevoix-Saguenay type, Axenfeld-Rieger syndrome type 1, Axenfeld-Rieger syndrome type 3, Hereditary cancer-predisposing syndrome, B lymphoblastic leukemia lymphoma, no ICD-O subtype, LEOPARD syndrome 1, Lymphoma, Metachondromatosis, Noonan syndrome 1, Noonan syndrome 3, Bainbridge-Ropers syndrome, BAKER-GORDON SYNDROME, SYT1-associated neurodevelopmental disorder, Baller-Gerold syndrome, Inborn genetic diseases, Rapadilino syndrome, Rothmund-Thomson syndrome, BAP1 Cancer Syndrome, Tumor susceptibility linked to germline BAP1 mutations, Baraitser-Winter syndrome 1, Baraitser-Winter Syndrome 2, Deafness, autosomal dominant 20, Barakat syndrome, Bardet-Biedl syndrome, Bardet-Biedl syndrome 1, Bardet-Biedl syndrome 10, Bardet-Biedl syndrome 13, Bardet-Biedl syndrome 14, Bardet-Biedl syndrome 16, Bardet-Biedl syndrome 17, Bardet-Biedl syndrome 18, Bardet-Biedl syndrome 2, Bardet-Biedl syndrome 21, Bardet-Biedl syndrome 3, Bardet-Biedl syndrome 4, Bardet-Biedl syndrome 5, Bardet-Biedl syndrome 6, Bardet-Biedl syndrome 7, Bardet-Biedl syndrome 9, COACH syndrome, Joubert syndrome 6, Bardet-biedl syndrome 2/6, digenic, Bardet-biedl syndrome 6/10, digenic, Inborn genetic diseases, Retinitis pigmentosa, Usher syndrome, Foot polydactyly, High-frequency hearing impairment, Intellectual disability, Macular degeneration, Postaxial hand polydactyly, Retinal dystrophy, Blindness, Cystic renal dysplasia, Leber congenital amaurosis 10, Meckel syndrome type 4, Meckel-Gruber syndrome, Nephronophthisis, Occipital encephalocele, Senior-Loken syndrome 6, Senior-Loken syndrome 7, Chronic kidney disease, Global developmental delay, Joubert syndrome 28, Limb undergrowth, Rotary nystagmus, Joubert syndrome 5, Retinal dystrophy, Retinitis pigmentosa 74, Retinitis pigmentosa 55, McKusick Kaufman syndrome, Inborn genetic diseases, Senior-Loken syndrome 7, Bare lymphocyte syndrome 2, Barrett esophagus/esophageal adenocarcinoma, Bartter syndrome type 3, Bartter syndrome type 4, Bartter syndrome, type 1, antenatal, Nephrocalcinosis, Nephrolithiasis, Bartter syndrome, type 2, antenatal, Bartter syndrome, type 3, with hypocalciuria, Bartter syndrome, type 5, antenatal, transient, Basal cell carcinoma, somatic, Basal ganglia calcification, idiopathic, 4, Basal ganglia calcification, idiopathic, 6, Basal laminar drusen, Basan syndrome, Basel-Vanagaite-Smirin-Yosef syndrome, BBS2-Related Disorders, Beaded hair, Beaulieu-Boycott-Innes syndrome, Becker muscular dystrophy, Dilated cardiomyopathy 3B, Duchenne muscular dystrophy, Beckwith-Wiedemann syndrome, Sotos syndrome 1, Benign familial hematuria, Benign familial neonatal seizures 1, Benign scapuloperoneal muscular dystrophy with cardiomyopathy, Congenital muscular dystrophy, LMNA-related, Limb-girdle muscular dystrophy, type 1B, Bent bone dysplasia syndrome, Bernard-Soulier syndrome type C, Macrothrombocytopenia, Bernard-Soulier syndrome, type A1, Bestrophinopathy, autosomal recessive, beta Thalassemia, Beta thalassemia intermedia, Beta-plus-thalassemia, Beta thalassemia major, beta\hat{ }0 \hat{ } Thalassemia, Beta-D-mannosidosis, Beta-Houston-thalassemia, Beta-malay-thalassemia, Beta-thalassemia, dominant inclusion body type, Bethlem myopathy 1, Dystonia 27, Ullrich congenital muscular dystrophy 1, BETHLEM MYOPATHY 1, AUTOSOMAL RECESSIVE, Beukes hip dysplasia, SPONDYLOEPIMETAPHYSEAL DYSPLASIA, DI ROCCO TYPE, Bietti crystalline corneoretinal dystrophy, Stargardt disease 1, Bifunctional peroxisomal enzyme deficiency, Perrault syndrome, Perrault syndrome 1, Bilateral conductive hearing impairment, Bilateral sensorineural hearing impairment, Deafness, Deafness, autosomal dominant 3a, Deafness, autosomal recessive 1A, Deafness, digenic, GJB2/GJB6, Hystrix-like ichthyosis with deafness, Keratitis-ichthyosis-deafness syndrome, autosomal dominant, Keratoderma palmoplantar deafness, Knuckle pads, deafness AND leukonychia syndrome, Mutilating keratoderma, Nonsyndromic Hearing Loss, Recessive, Nonsyndromic hearing loss and deafness, Rare genetic deafness, Severe sensorineural hearing impairment, Bilateral right-sidedness sequence, Bile acid synthesis defect, congenital, 1, Bile acid synthesis defect, congenital, 6, Biotinidase deficiency, Biotin-thiamine-responsive basal ganglia disease, Birk Barel mental retardation dysmorphism syndrome, Cardiac conduction disease with or without dilated cardiomyopathy, Cardiac valvular defect, developmental, Cardiac valvular dysplasia, X-linked, Cardio-facio-cutaneous syndrome, Cardiofaciocutaneous syndrome 3, Cardio-facio-cutaneous syndrome 1, Inborn genetic diseases, Rasopathy, LEOPARD syndrome 3, Lung cancer, Noonan syndrome 1, Noonan syndrome 7, Noonan syndrome and Noonan-related syndrome, Glioblastoma, Lung adenocarcinoma, Multiple myeloma, Transitional cell carcinoma of the bladder, Cutaneous melanoma, Neoplasm, Non-small cell lung cancer, Cardiomyopathy, Cardiomyopathy, dilated, with woolly hair, keratoderma, and tooth agenesis, Cardiomyopathy, familial hypertrophic, 26, Dilated Cardiomyopathy, Dominant, Myofibrillar myopathy, filamin C-related, Myopathy, distal, 4, Cardiomyopathy, familial restrictive, 5, Cardiomyopathy, mitochondrial, Cardiovascular phenotype, Familial hypertrophic cardiomyopathy 4, Hypertrophic cardiomyopathy, Left ventricular noncompaction, Familial hypertrophic cardiomyopathy 6, Glycogen storage disease of heart, lethal congenital, Primary familial hypertrophic cardiomyopathy, Wolff-Parkinson-White pattern, Dilated cardiomyopathy 1G, Hereditary myopathy with early respiratory failure, Limb-girdle muscular dystrophy, type 2J, Primary dilated cardiomyopathy, Familial dilated cardiomyopathy, Familial hypertrophic cardiomyopathy 1, Congenital long QT syndrome, Jervell and Lange-Nielsen syndrome 1, Long QT syndrome, Long QT syndrome 1, Congenital myopathy with fiber type disproportion, Dilated cardiomyopathy 1S, Myopathy, distal, 1, Myopathy, myosin storage, autosomal recessive, Myosin storage myopathy, Scapuloperoneal myopathy, MYH7-related, Primary dilated cardiomyopathy, Ebstein anomaly of the tricuspid valve, Left ventricular noncompaction 10, Familial restrictive cardiomyopathy 3, Ehlers-Danlos syndrome, hydroxylysine-deficient, Ehlers-Danlos syndrome, type 4, Fabry disease, Fabry disease, cardiac variant, Myopathy, distal, 1, MYBPC3-Related Disorders, Inborn genetic diseases, Thoracic aortic aneurysm and aortic dissection, Carney complex, type 1, Carnitine acylcarnitine translocase deficiency, Carnitine palmitoyltransferase I deficiency, Carnitine palmitoyltransferase II deficiency, Encephalopathy, acute, infection-induced, 4, susceptibility to, Carpenter syndrome 2, Carpenter syndrome 1, Cataract 1, Cataract 15, multiple types, Cataract 2, Coppock-like, Cataract 21, multiple types, Cataract 23, multiple types, Congenital cataract, Cataract 39, multiple types, Cataract 4, Cataract 44, Cataract Hutterite type, Cataract, autosomal dominant, Cataract, autosomal dominant, multiple types, with microcornea, Cataract, autosomal recessive congenital 4, Cataract, autosomal recessive congenital 5, Sengers syndrome, Cataract, congenital nuclear, autosomal recessive 2, Cataract, microphthalmia and nystagmus, Catecholaminergic polymorphic ventricular tachycardia, Catecholaminergic polymorphic ventricular tachycardia type 1, DOORS syndrome, Deafness, autosomal dominant 65, Epileptic encephalopathy, early infantile, 1, Cavernous hemangioma, CDH23-Related Disorders, Deafness, autosomal recessive 12, PITUITARY ADENOMA 5, MULTIPLE TYPES, Rare genetic deafness, Usher syndrome, type 1D, Central scotoma, Macular degeneration, Retinal atrophy, Stargardt disease 1, Visual impairment, Centromeric instability of chromosomes 1,9 and 16 and immunodeficiency, Cerebellar ataxia and mental retardation with quadrupedal locomotion 1, Cerebellar ataxia, mental retardation, and dysequilibrium syndrome 1, Cerebellar ataxia, Cerebellar atrophy, Dysdiadochokinesis, Dysmetria, Nystagmus, Slightly reduced reflexes, Slurred speech, Global developmental delay, Microcephaly, Myoclonus, Seizures, CEREBELLAR ATROPHY WITH SEIZURES AND VARIABLE DEVELOPMENTAL DELAY, Congenital anomalies of kidney and urinary tract, Cerebellar cortical atrophy, Dystonia, Cerebellar vermis hypoplasia, Early infantile epileptic encephalopathy, Hartsfield syndrome, Xia-Gibbs syndrome, CEREBELLAR, OCULAR, CRANIOFACIAL, AND GENITAL SYNDROME, Cerebellofaciodental syndrome, CEREBRAL AMYLOID ANGIOPATHY, APP-RELATED, PIEDMONT VARIANT, CEREBRAL AMYLOID ANGIOPATHY, PRNP-RELATED, Genetic prion diseases, Cerebral autosomal dominant arteriopathy with subcortical infarcts and leukoencephalopathy, Cerebral autosomal recessive arteriopathy with subcortical infarcts and leukoencephalopathy, Cerebral calcification, Dilatation, Interstitial pneumonitis, Liver cirrhosis, Rajab syndrome, Cerebral cavernous malformation, Cerebral cavernous malformations 1, Cerebral cavernous malformations 2, Cerebral cavernous malformations 3, Distal spinal muscular atrophy, Gait ataxia, Hereditary spastic paraplegia, Memory impairment, Spastic paraparesis, Spastic paraplegia, Spastic paraplegia 7, Cerebral creatine deficiency syndrome, Deficiency of guanidinoacetate methyltransferase, Cerebral dysgenesis, neuropathy, ichthyosis, and palmoplantar keratoderma syndrome, Cerebral folate deficiency, Cerebral hypomyelination, Elbow flexion contracture, Intellectual disability, Knee flexion contracture, Muscular hypotonia, Cerebral hypoplasia, Cerebral palsy, spastic quadriplegic, 1, Cerebral visual impairment and intellectual disability, Mental retardation, autosomal recessive 42, Cerebro-costo-mandibular syndrome, Cerebrooculofacioskeletal syndrome 2, Trichothiodystrophy 1, photosensitive, Xeroderma pigmentosum, group D, Cerebrooculofacioskeletal syndrome 3, Cerebroretinal microangiopathy with calcifications and cysts, Cerebroretinal microangiopathy with calcifications and cysts 1, Dyskeratosis congenita, Ceroid lipofuscinosis neuronal 1, Neuronal ceroid lipofuscinosis, Ceroid lipofuscinosis neuronal 10, Ceroid lipofuscinosis neuronal 2, Childhood-onset autosomal recessive slowly progressive spinocerebellar ataxia, Ceroid lipofuscinosis neuronal 4B autosomal dominant, Ceroid lipofuscinosis neuronal 5, Ceroid lipofuscinosis neuronal 6, Ceroid lipofuscinosis neuronal 7, Macular dystrophy with central cone involvement, Ceroid lipofuscinosis neuronal 8, Ceroid lipofuscinosis, neuronal, 8, northern epilepsy variant, Ceroid lipofuscinosis, neuronal, 11, Frontotemporal dementia, ubiquitin-positive, Primary progressive aphasia, Ceroid lipofuscinosis, neuronal, 13, Ceroid lipofuscinosis, neuronal, 3, protracted, Juvenile neuronal ceroid lipofuscinosis, Ceruloplasmin belfast, Deficiency of ferroxidase, CFHR5 deficiency, Chediak-Higashi syndrome, Charcot-Marie-Tooth disease, Charcot-Marie-Tooth disease dominant intermediate 3, Charcot-Marie-Tooth disease type 21, Charcot-Marie-Tooth disease type 2J, Charcot-Marie-Tooth disease, demyelinating, type 1b, Congenital hypomyelinating neuropathy 1, autosomal recessive, Dejerine-Sottas disease, Dejerine-Sottas syndrome, autosomal dominant, Roussy-Levy syndrome, Charcot-Marie-Tooth disease type 2C, Neuromuscular Diseases, Parastremmatic dwarfism, Skeletal dysplasia, Spondylometaphyseal dysplasia, Kozlowski type, Charcot-Marie-Tooth disease type 2E, Distal hereditary motor neuronopathy type 2B, Charcot-Marie-Tooth disease type 2K, Charcot-Marie-Tooth disease, axonal, with vocal cord paresis, autosomal recessive, Charcot-Marie-Tooth disease, recessive intermediate A, Charcot-Marie-Tooth disease, type 4A, Neuropathy, axonal, with vocal cord paresis, autosomal recessive, Charcot-Marie-Tooth disease type 4, Charcot-Marie-Tooth disease, demyelinating, type 4F, Charcot-Marie-Tooth disease, dominant intermediate B, Myopathy, centronuclear, Myopathy, centronuclear, 1, Charcot-Marie-Tooth disease, dominant intermediate E, Focal segmental glomerulosclerosis 5, Charcot-Marie-Tooth disease, dominant intermediate G, Charcot-Marie-Tooth disease, recessive intermediate c, Charcot-Marie-Tooth disease, type 1C, Charcot-Marie-Tooth disease, axonal type 2V, Mucopolysaccharidosis, MPS-III-B, Charcot-Marie-Tooth disease, axonal type 2X, Spastic paraplegia 11, autosomal recessive, Charcot-Marie-Tooth disease, axonal, autosomal recessive, type 2A2B, Hereditary motor and sensory neuropathy with optic atrophy, Charcot-Marie-Tooth disease, axonal, type 2CC, Charcot-Marie-Tooth disease, axonal, type 2DD, MPV17-Related Disorders, Navajo neurohepatopathy, Charcot-Marie-Tooth disease, axonal, type 20, Spinal muscular atrophy, lower extremity predominant 1, autosomal dominant, Charcot-Marie-Tooth disease, axonal, type 2Q, Charcot-Marie-Tooth disease, axonal, type 2R, Charcot-Marie-Tooth disease, axonal, type 2S, Spinal muscular atrophy, distal, autosomal recessive, 1, Werdnig-Hoffmann disease, Charcot-Marie-Tooth disease, axonal, type 2T, Charcot-Marie-Tooth disease, axonal, type 2w, Charcot-Marie-Tooth disease, axonal, type 2z, Charcot-Marie-Tooth disease, demyelinating, type 1d, Charcot-Marie-Tooth disease, type I, Charcot-Marie-Tooth disease, demyelinating, type 1f, Charcot-Marie-Tooth disease, demyelinating, type 1G, Charcot-Marie-Tooth disease, type 4B1, Charcot-Marie-Tooth disease, type 4B3, Charcot-Marie-Tooth disease, type 4C, Charcot-Marie-Tooth disease, type IA, Charcot-Marie-Tooth disease, X-linked recessive, type 5, Charcot-Marie-Tooth disease type 2, Distal spinal muscular atrophy, congenital nonprogressive, Scapuloperoneal spinal muscular atrophy, Charcot-Marie-Tooth Neuropathy X, X-linked hereditary motor and sensory neuropathy, CHARGE association, Kallmann syndrome 5, Chest pain, Child syndrome, Childhood Onset Dystonias, Optic atrophy, Childhood-Onset Schizophrenia, Cholecystitis, Cholestanol storage disease, Cholestasis, benign recurrent intrahepatic 1, Progressive intrahepatic cholestasis, Cholestasis, intrahepatic, of pregnancy 3, Progressive familial intrahepatic cholestasis 3, Cholesterol monooxygenase (side-chain cleaving) deficiency, Chondrodysplasia punctata 1, X-linked recessive, Chondrodysplasia punctata 2 X-linked dominant, Chondrodysplasia punctata 2, X-linked dominant, atypical, Chondrodysplasia with joint dislocations, GPAPP type, Chondrodysplasia with platyspondyly, distinctive brachydactyly, hydrocephaly, and microphthalmia, Chondroectodermal dysplasia, Curry-Hall syndrome, Ellis-van Creveld Syndrome, Short rib-polydactyly syndrome, Majewski type, Chorea, Epileptic encephalopathy, early infantile, 37, Progressive encephalopathy, Choreoacanthocytosis, Choreoathetosis, hypothyroidism, and neonatal respiratory distress, Choroidal dystrophy, central areolar 2, Cone/cone-rod dystrophy, Progressive cone dystrophy (without rod involvement), Choroidal sclerosis, Cone-rod dystrophy 6, Leber congenital amaurosis, Leber congenital amaurosis 1, NIGHT BLINDNESS, CONGENITAL STATIONARY, TYPE 11, Choroideremia, Christianson syndrome, CHRNG-Related Disorders, Lethal multiple pterygium syndrome, Multiple pterygium syndrome Escobar type, Chromophobe renal cell carcinoma, Renal cysts and diabetes syndrome, Chromosome 2q32-q33 deletion syndrome, Cleft palate, isolated, History of neurodevelopmental disorder, Chromosome 9q deletion syndrome, Chromosome Xq28 deletion syndrome, Chronic granulomatous disease, autosomal recessive cytochrome b-positive, type 1, Granulomatous disease, chronic, autosomal recessive, cytochrome b-positive, type III, Chronic granulomatous disease, autosomal recessive cytochrome b-positive, type 2, Chronic granulomatous disease, X-linked, Cryopyrin associated periodic syndrome, DEAFNESS, AUTOSOMAL DOMINANT 34, WITH OR WITHOUT INFLAMMATION, Familial amyloid nephropathy with urticaria AND deafness, Familial cold urticaria, Keratitis fugax hereditaria, Chronic intestinal pseudoobstruction, Megacystis, Visceral myopathy, Visceral neuropathy, familial, autosomal dominant, Chronic myelogenous leukemia, Chronic pancreatitis, Hereditary pancreatitis, Tropical calcific pancreatitis, Chronic progressive multiple sclerosis, Ciliary dyskinesia, primary, 10, Kartagener syndrome, Ciliary dyskinesia, primary, 11, Primary ciliary dyskinesia, Ciliary dyskinesia, primary, 13, Ciliary dyskinesia, primary, 15, Ciliary dyskinesia, primary, 16, Ciliary dyskinesia, primary, 17, Ciliary dyskinesia, primary, 18, Ciliary dyskinesia, primary, 19, Ciliary dyskinesia, primary, 2, Ciliary dyskinesia, primary, 20, Ciliary dyskinesia, primary, 21, Ciliary dyskinesia, primary, 22, Ciliary dyskinesia, primary, 26, Ciliary dyskinesia, primary, 28, Ciliary dyskinesia, primary, 29, Ciliary dyskinesia, primary, 3, Ciliary dyskinesia, primary, 30, Ciliary dyskinesia, primary, 32, Cone-rod dystrophy 2, Cone-rod dystrophy 20, Cone-rod dystrophy 3, Retinal dystrophy, Retinitis pigmentosa 19, Stargardt disease 1, Progressive cone dystrophy (without rod involvement), Cone-rod dystrophy amelogenesis imperfecta, Cone-rod dystrophy and hearing loss, Juvenile retinitis pigmentosa, Cone-rod dystrophy, X-linked 1, Retinal pigment epithelial atrophy, Retinitis pigmentosa 26, Rare genetic deafness, Usher syndrome, type 2A, Retinitis pigmentosa 39, Congenital absence of salivary gland, Levy-Hollister syndrome, Congenital adrenal hypoplasia, X-linked, Congenital amegakaryocytic thrombocytopenia, MPL-Related Disorders, Myelofibrosis, Thrombocytopenia, Thrombocytosis, benign familial microcytic, essential thrombocytemia, Hematologic neoplasm, Myelofibrosis with myeloid metaplasia, Thrombocythemia 2, somatic, Congenital bilateral absence of the vas deferens, Vas deferens, congenital bilateral aplasia of, X-linked, Congenital cataract, Neurodevelopmental delay, Severe sensorineural hearing impairment, Congenital cataracts, hearing loss, and neurodegeneration, Congenital central hypoventilation, Congenital contractural arachnodactyly, Congenital contractures of the limbs and face, hypotonia, and developmental delay, Intellectual disability with episodic ataxia and congenital arthrogryposis, Congenital defect of folate absorption, Congenital diaphragmatic hernia, Cryptorchidism, Hypospadias, penile, Intrauterine growth retardation, Long philtrum, Microretrognathia, Pulmonary hypoplasia, Right ventricular hypertrophy, Single umbilical artery, Congenital disorder of deglycosylation, Inborn genetic diseases, Congenital disorder of glycosylation type 1 bb, Congenital disorder of glycosylation type 1C, Congenital disorder of glycosylation type 1D, Congenital disorder of glycosylation type 1E, Congenital disorder of glycosylation type 1H, Congenital disorder of glycosylation type 1I, Congenital disorder of glycosylation type 1J, Congenital disorder of glycosylation type 1K, Congenital disorder of glycosylation type 1M, Congenital disorder of glycosylation type 1N, Congenital disorder of glycosylation type 1O, Congenital disorder of glycosylation type 1P, Congenital disorder of glycosylation type 1t, Congenital disorder of glycosylation type 1y, Congenital disorder of glycosylation type 2C, Congenital disorder of glycosylation type 2J, Congenital disorder of glycosylation with defective fucosylation 2, CONGENITAL DISORDER OF GLYCOSYLATION, TYPE IIm, CONGENITAL DISORDER OF GLYCOSYLATION, TYPE IIn, CONGENITAL DISORDER OF GLYCOSYLATION, TYPE IIo, Congenital disorders of glycosylation type II, CONGENITAL DISORDER OF GLYCOSYLATION, TYPE lip, Congenital dyserythropoietic anemia, type I, Congenital dyserythropoietic anemia, type II, Cowden syndrome 7, Congenital erythropoietic porphyria, Congenital generalized lipodystrophy type 1, Congenital generalized lipodystrophy type 2, Encephalopathy, progressive, with or without lipodystrophy, Congenital glaucoma, Glaucoma 3, primary congenital, A, Congenital glucose-galactose malabsorption, Congenital heart defects and ectodermal dysplasia, Congenital heart defects, multiple types, 1, X-linked, Congenital heart defects, multiple types, 2, Congenital heart defects, multiple types, 4, Congenital heart disease, Congenital hydrocephalus 1, Congenital hyperammonemia, type I, Congenital hypomyelinating neuropathy 1, autosomal dominant, Congenital hypomyelinating neuropathy 1, autosomal recessive, Congenital hypomyelinating neuropathy 3, Congenital hypothyroidism, Hypothyroidism, congenital, nongoitrous, 1, Congenital ATN1 related disorder, Congenital lactase deficiency, Congenital long QT syndrome, Long QT syndrome, Long QT syndrome 1, Long QT syndrome 1/2, digenic, Long QT syndrome 2, Long QT syndrome 2/5, Long QT syndrome, bradycardia-induced, Long QT syndrome, LQT1 subtype, Long qt syndrome 3/6, digenic, Long QT syndrome 3, Congenital microvillous atrophy, Congenital muscular dystrophy due to partial LAMA2 deficiency, Laminin alpha 2-related dystrophy, Merosin deficient congenital muscular dystrophy, Congenital muscular dystrophy-dystroglycanopathy (with or without mental retardation) type B5, Difficulty climbing stairs, Difficulty standing, Difficulty walking, Gait imbalance, Headache, Limb-girdle muscular dystrophy, Limb-girdle muscular dystrophy-dystroglycanopathy, type C5, Muscle weakness, Muscular dystrophy-dystroglycanopathy (congenital with brain and eye anomalies), type A, 1, Paresthesia, Scapular winging, Walker-Warburg congenital muscular dystrophy, Congenital muscular dystrophy-dystroglycanopathy with brain and eye anomalies type A5, FKRP-Related Disorder, Congenital muscular dystrophy-dystroglycanopathy with brain and eye anomalies, type A2, Congenital muscular dystrophy-dystroglycanopathy with mental retardation, type B2, Congenital muscular dystrophy-dystroglycanopathy with brain and eye anomalies, type A3, Congenital muscular dystrophy-dystroglycanopathy with brain and eye anomalies, type A4, FKTN-Related Disorders, Fukuyama congenital muscular dystrophy, Congenital muscular dystrophy-dystroglycanopathy with brain and eye anomalies, type A6, Congenital muscular dystrophy-dystroglycanopathy with brain and eye anomalies, type A7, Congenital muscular dystrophy-dystroglycanopathy with mental retardation, type B1, POMT1-Related Disorders, Congenital muscular hypertrophy-cerebral syndrome, Congenital myasthenic syndrome, Endplate acetylcholinesterase deficiency, Familial hyperkalemic periodic paralysis, Hyperkalemic Periodic Paralysis Type 1, Hypokalemic periodic paralysis 1, Hypokalemic periodic paralysis, type 2, Paramyotonia congenita of von Eulenburg, Paramyotonia congenita/hyperkalemic periodic paralysis, Potassium aggravated myotonia, Congenital myopathy with fiber type disproportion, Myopathy, Central Core, Congenital myotonia, autosomal dominant form, Congenital myotonia, autosomal recessive form, Myotonia congenita, Congenital NAD deficiency disorder, VERTEBRAL, CARDIAC, RENAL, AND LIMB DEFECTS SYNDROME 1, Congenital nonprogressive myopathy with Moebius and Robin sequences, Congenital omphalocele, Deafness, X-linked 2, Deafness, autosomal dominant 3a, Deafness, autosomal recessive 1A, Hearing impairment, Hearing loss, Horseshoe kidney, Hystrix-like ichthyosis with deafness, Keratitis-ichthyosis-deafness syndrome, autosomal dominant, Keratoderma palmoplantar deafness, Knuckle pads, deafness AND leukonychia syndrome, Mutilating keratoderma, Short palpebral fissure, Congenital secretory diarrhea, chloride type, Congenital stationary night blindness, Pigmentary retinal dystrophy, Retinitis pigmentosa 4, Congenital Stromal Corneal Dystrophy, Congenital titinopathy, Myopathy, early-onset, with fatal cardiomyopathy, Conotruncal anomaly face syndrome/velocardiofacial syndrome, CONTRACTURES, PTERYGIA, AND VARIABLE SKELETAL FUSIONS SYNDROME 1B, Distal arthrogryposis type 8, Spondylocarpotarsal synostosis syndrome, Cornea plana 2, Corneal dystrophy, Corneal dystrophy and perceptive deafness, Corneal dystrophy, Fuchs endothelial 1, Corneal dystrophy, posterior polymorphous, 2, Corneal dystrophy, Fuchs endothelial, 6, Cornelia de Lange syndrome 1, Cornelia de Lange syndrome 3, Cornelia de Lange syndrome 4, Cornelia de Lange syndrome 5, Coronary artery disease, Diabetes, Hypertension, Hypertriglyceridemia, Coronary heart disease, Hyperalphalipoproteinemia 2, Corpus callosum agenesis, Mirror movements 1, Cortical dysplasia, complex, with other brain malformations 1, Fibrosis of extraocular muscles, congenital, 3a, with or without extraocular involvement, Cortical dysplasia, complex, with other brain malformations 2, Cortical dysplasia, complex, with other brain malformations 3, Cortical dysplasia, complex, with other brain malformations 4, Cortical dysplasia, complex, with other brain malformations 5, Cortisone reductase deficiency 1, Cowchock syndrome, Cowden syndrome 6, Cranioectodermal dysplasia, Cranioectodermal dysplasia 1, Cranioectodermal dysplasia 2, Cranioectodermal dysplasia 4, Senior-Loken syndrome 8, Craniofacial dysmorphism, skeletal anomalies, and mental retardation syndrome, Craniofrontonasal dysplasia, Craniometaphyseal dysplasia, autosomal dominant, Craniosynostosis 1, Saethre-Chotzen syndrome, Craniosynostosis 2, Craniosynostosis 3, Craniosynostosis 6, Craniosynostosis, nonsyndromic unicoronal, Mental retardation, autosomal dominant, CRB1-Related Disorders, Retinitis pigmentosa 12, Creatine deficiency, X-linked, Crigler Najjar syndrome, type 1, Crisponi/Cold-induced sweating syndrome, Crouzon syndrome, FGFR2 related craniosynostosis, Jackson-Weiss syndrome, Pfeiffer syndrome, Treacher Collins syndrome, Cryopyrin associated periodic syndrome, Familial amyloid nephropathy with urticaria AND deafness, Familial cold urticaria, Cryptophthalmos syndrome, Culler-Jones syndrome, Holoprosencephaly 9, Currarino triad, Cutaneous melanoma, Dysgerminoma, Gastrointestinal stroma tumor, Hematologic neoplasm, Malignant tumor of testis, Thymoma, Cutis laxa with osteodystrophy, Cutis laxa with severe pulmonary, gastrointestinal, and urinary abnormalities, Cutis laxa, autosomal dominant 3, Cutis laxa, X-linked, Distal spinal muscular atrophy, X-linked 3, Menkes kinky-hair syndrome, Cutis laxa-corneal clouding-oligophrenia syndrome, Cyclical neutropenia, Neutropenia, severe congenital 1, autosomal dominant, Cylindromatosis, familial, Cystathioninuria, Cystic fibrosis, Sweat chloride elevation without cystic fibrosis, Cystinosis, Cystinosis, ocular nonnephropathic, Juvenile nephropathic cystinosis, Nephropathic cystinosis, Cystinuria, D-2-hydroxyglutaric aciduria, D-2-hydroxyglutaric aciduria 1, Dandy-Walker like malformation with atrioventricular septal defect, Danon disease, Hypertrophic cardiomyopathy, Primary dilated cardiomyopathy, Darier disease, acral hemorrhagic type, Deafness, Deafness and myopia, Deafness enamel hypoplasia nail defects, Leber congenital amaurosis, Peroxisomal disorder, Peroxisome biogenesis disorder 1A (Zellweger), Peroxisome biogenesis disorder 1B, Peroxisome biogenesis disorders, Zellweger syndrome spectrum, Deafness with labyrinthine aplasia microtia and microdontia (LAMM), Deafness, autosomal dominant 1, Deafness, autosomal dominant 11, Deafness, autosomal dominant 12, Deafness, autosomal dominant 15, Deafness, autosomal dominant 22, Deafness, autosomal dominant 36, Deafness, autosomal dominant 40, Deafness, autosomal dominant 4b, Deafness, autosomal dominant 5, Deafness, autosomal dominant 65, Deafness, autosomal dominant 67, Deafness, autosomal dominant 69, Deafness, autosomal dominant 9, Nonsyndromic hearing loss and deafness, Deafness, autosomal dominant nonsyndromic sensorineural 17, MYH9-related disorder, Macrothrombocytopenia and granulocyte inclusions with or without nephritis or sensorineural hearing loss, Deafness, autosomal recessive, Deafness, autosomal recessive 101, Deafness, autosomal recessive 103, DEAFNESS, AUTOSOMAL RECESSIVE 106, DEAFNESS, AUTOSOMAL RECESSIVE 107, DEAFNESS, AUTOSOMAL RECESSIVE 114, Deafness, autosomal recessive 12, PITUITARY ADENOMA 5, MULTIPLE TYPES, Usher syndrome, type 1D, Deafness, autosomal recessive 15, Deafness, autosomal recessive 16, Deafness, autosomal recessive 18, Retinitis pigmentosa, Usher syndrome, Deafness, autosomal recessive 18b, Deafness, autosomal recessive 2, Deafness, autosomal recessive 21, Deafness, autosomal recessive 22, Deafness, autosomal recessive 23, Deafness, autosomal recessive 25, Deafness, autosomal recessive 28, Deafness, autosomal recessive 29, Perrault syndrome, Deafness, autosomal recessive 3, Deafness, autosomal recessive 30, Deafness, autosomal recessive 49, Deafness, autosomal recessive 53, Deafness, autosomal recessive 6, Deafness, autosomal recessive 61, Deafness, autosomal recessive 66, Deafness, autosomal recessive 68, Deafness, autosomal recessive 7, Deafness, autosomal recessive 74, Deafness, autosomal recessive 76, Deafness, autosomal recessive 77, Deafness, autosomal recessive 79, Deafness, autosomal recessive 8, Deafness, autosomal recessive 84, Deafness, autosomal recessive 86, Deafness, autosomal recessive 89, Deafness, autosomal recessive 9, Deafness, autosomal recessive 97, Deafness, X-linked 4, Deafness, Y-linked 2, Deficiency of 2-methylbutyryl-CoA dehydrogenase, Deficiency of acetyl-CoA acetyltransferase, Deficiency of alpha-mannosidase, Deficiency of aromatic-L-amino-acid decarboxylase, Deficiency of bisphosphoglycerate mutase, Deficiency of butyrylcholine esterase, Deficiency of butyryl-CoA dehydrogenase, Deficiency of ferroxidase, Hemosiderosis, systemic, due to aceruloplasminemia, Deficiency of galactokinase, Deficiency of hydroxymethylglutaryl-CoA lyase, Deficiency of iodide peroxidase, Deficiency of pyrroline-5-carboxylate reductase, Deficiency of ribose-5-phosphate isomerase, Deficiency of steroid 11-beta-monooxygenase, Hyperaldosteronism, familial, type I, Deficiency of steroid 17-alpha-monooxygenase, Deficiency of transaldolase, Deficiency of UDPglucose-hexose-1-phosphate uridylyltransferase, Deficiency of UDPglucose-hexose-1-phosphate uridylyltransferase, Dejerine-Sottas disease, Dejerine-Sottas syndrome, autosomal dominant, Delayed gross motor development, Delayed speech and language development, Global developmental delay, Intellectual disability, Muscular hypotonia, Neonatal hypotonia, Sleep apnea, Xia-Gibbs syndrome, Early infantile epileptic encephalopathy 4, Generalized hypotonia, Horizontal nystagmus, Infantile spasms, Muscular hypotonia of the trunk, Strabismus, NEURODEVELOPMENTAL DISORDER WITH SEVERE MOTOR IMPAIRMENT AND ABSENT LANGUAGE, Hearing impairment, Microcephaly, Mental retardation, autosomal dominant 31, Seizures, delta Thalassemia, Delta-zero-thalassemia, knossos type, Dementia, Frontotemporal dementia, Memory impairment, Mental deterioration, Dendritic cell, monocyte, B lymphocyte, and natural killer lymphocyte deficiency, Dent disease 1, Dentin dysplasia, type I, with extreme microdontia and misshapen teeth, Dermatitis, atopic, 2, susceptibility to, Ichthyosis vulgaris, Dermatopathia pigmentosa reticularis, Desanto-shinawi syndrome, Desbuquois dysplasia 1, Desbuquois dysplasia 2, Desmosterolosis, Deuteranopia, DEVELOPMENTAL DELAY AND SEIZURES WITH OR WITHOUT MOVEMENT ABNORMALITIES, Retinitis pigmentosa, Retinitis pigmentosa 59, DEVELOPMENTAL DELAY WITH OR WITHOUT DYSMORPHIC FACIES AND AUTISM, DEVELOPMENTAL DELAY WITH VARIABLE INTELLECTUAL IMPAIRMENT AND BEHAVIORAL ABNORMALITIES, Inborn genetic diseases, Neurodevelopmental abnormality, DEVELOPMENTAL DELAY, INTELLECTUAL DISABILITY, OBESITY, AND DYSMORPHISM, DFNA 2 Nonsyndromic Hearing Loss, Diabetes insipidus, nephrogenic, autosomal recessive, Diabetes mellitus, permanent neonatal, with neurologic features, Permanent neonatal diabetes mellitus, Diamond-Blackfan anemia, Diamond-Blackfan anemia 1, Diamond-Blackfan anemia 10, Diamond-Blackfan anemia 13, Diamond-Blackfan anemia 14 with mandibulofacial dysostosis, Diamond-Blackfan anemia 15 with mandibulofacial dysostosis, Diamond-Blackfan anemia 19, Diamond-Blackfan anemia 3, Diamond-Blackfan anemia 4, Diamond-Blackfan anemia 7, GATA-1-related thrombocytopenia with dyserythropoiesis, Diaphanospondylodysostosis, Diaphragmatic hernia 3, Double outlet right ventricle, Diaphyseal dysplasia, Diarrhea 3, secretory sodium, congenital, syndromic, Diarrhea 4, malabsorptive, congenital, Diarrhea 7, Diarrhea 8, secretory sodium, congenital, Diastrophic dysplasia, Diastrophic dysplasia, broad bone-platyspondylic variant, Diffuse interstitial pulmonary fibrosis, Primary interstitial lung disease specific to childhood due to pulmonary surfactant protein anomalies, Pulmonary arterial hypertension, Pulmonary insufficiency, Respiratory insufficiency, Surfactant metabolism dysfunction, pulmonary, 3, Diffuse mesangial sclerosis, Drash syndrome, Frasier syndrome, Meacham syndrome, Wilms tumor 1, Diffuse palmoplantar keratoderma, Bothnian type, DiGeorge sequence, Digital arthropathy-brachydactyly, familial, Dihydropteridine reductase deficiency, Dihydropyrimidine dehydrogenase deficiency, Dilatation of the ascending aorta, Dolichocephaly, High palate, Mitral valve prolapse, Myopia, Pes planus, Scoliosis, Dilated cardiomyopathy 1A, Familial partial lipodystrophy 2, Primary dilated cardiomyopathy, Dilated cardiomyopathy 1BB, Dilated cardiomyopathy 1C, Dilated cardiomyopathy 1CC, Dilated cardiomyopathy 1DD, Dilated cardiomyopathy 1E, Dilated cardiomyopathy 1FF, Dilated cardiomyopathy 1G, Distal myopathy Markesbery-Griggs type, Familial dilated cardiomyopathy, Familial hypertrophic cardiomyopathy 9, Hereditary myopathy with early respiratory failure, Limb-girdle muscular dystrophy, type 2J, Myopathy, early-onset, with fatal cardiomyopathy, Dilated cardiomyopathy 1HH, Myofibrillar myopathy, BAG3-related, Dilated cardiomyopathy 1O, Hypertrichotic osteochondrodysplasia, Dilated cardiomyopathy 1S, Left ventricular noncompaction, Left ventricular noncompaction cardiomyopathy, Dilated cardiomyopathy 1X, Dilated cardiomyopathy 1Y, Dilated cardiomyopathy 3B, Duchenne muscular dystrophy, Dilated cardiomyopathy with woolly hair and keratoderma, Dilated Cardiomyopathy, Dominant, Disordered steroidogenesis due to cytochrome p450 oxidoreductase deficiency, Disseminated atypical mycobacterial infection, Distal arthrogryposis type 1A, Nemaline myopathy 4, Distal arthrogryposis type 5D, Distal arthrogryposis type 8, Distal hereditary motor neuronopathy 2D, Distal hereditary motor neuronopathy type 5, Growth delay, Intrauterine growth retardation, Pyridoxal 5′-phosphate-dependent epilepsy, Distal spinal muscular atrophy, Distichiasis-lymphedema syndrome, Dominant dystrophic epidermolysis bullosa with absence of skin, Dystrophic epidermolysis bullosa, Epidermolysis bullosa pruriginosa, Generalized dominant dystrophic epidermolysis bullosa, Nail disorder, nonsyndromic congenital, 8, Pretibial epidermolysis bullosa, Transient bullous dermolysis of the newborn, Donnai Barrow syndrome, DOORS syndrome, Dopamine beta hydroxylase deficiency, Dowling-Degos disease 1, Dowling-Degos disease 2, Dowling-degos disease 4, Duane retraction syndrome 3 with or without deafness, Duane syndrome type 1, Duane syndrome type 2, Duane-radial ray syndrome, Dubin-Johnson syndrome, Dyggve-Melchior-Clausen syndrome, Smith-McCort dysplasia 1, Dyschromatosis universalis hereditaria 1, Dyserythropoietic anemia with thrombocytopenia, Dysferlinopathy, Limb-girdle muscular dystrophy, type 2B, Miyoshi muscular dystrophy 1, Myopathy, distal, with anterior tibial onset, Dyskeratosis congenita, Dyskeratosis congenita autosomal dominant, Dyskeratosis congenita, autosomal dominant, 2, Dyskeratosis congenita, autosomal dominant, 3, Dyskeratosis congenita autosomal recessive 1, Dyskeratosis congenita, autosomal recessive 2, Dyskeratosis congenita X-linked, Hoyeraal Hreidarsson syndrome, Dyskeratosis congenita, autosomal dominant 6, Dyskeratosis congenita, autosomal recessive 7, Idiopathic fibrosing alveolitis, chronic form, Pulmonary fibrosis and/or bone marrow failure, telomere-related, 1, Revesz syndrome, Dyskinesia, familial, with facial myokymia, Dyskinesia, limb and orofacial, infantile-onset, Dysmorphic features, Epilepsy, Intellectual developmental disorder with dysmorphic facies, seizures, and distal limb anomalies, Learning difficulty, dysmorphy, Dysostosis multiplex, Mucopolysaccharidosis type I, Mucopolysaccharidosis, MPS-I-H/S, Mucopolysaccharidosis, MPS-I-S, Dysplastic pulmonary valve, Failure to thrive, Juvenile myelomonocytic leukemia, LEOPARD syndrome 1, Metachondromatosis, Noonan syndrome, Noonan syndrome 1, Patent ductus arteriosus, Rasopathy, Right ventricular hypertrophy, Secundum atrial septal defect, Tricuspid regurgitation, Dystonia 10, Paroxysmal kinesigenic dyskinesia, Dystonia 12, Dystonia 16, Dystonia 2, torsion, autosomal recessive, Dystonia 24, Dystonia 25, Dystonia 28, childhood-onset, Dystonia 5, Dopa-responsive type, GTP cyclohydrolase I deficiency, Dystonia 9, Epilepsy, idiopathic generalized, susceptibility to, 12, GLUT1 deficiency syndrome 1, GLUT1 deficiency syndrome 1, autosomal recessive, GLUT1 deficiency syndrome 2, Stomatin-deficient cryohydrocytosis with neurologic defects, Segawa syndrome, autosomal recessive, Sepiapterin reductase deficiency, Early infantile epileptic encephalopathy, Severe myoclonic epilepsy in infancy, Early onset focal segmental glomerulosclerosis, GALLOWAY-MOWAT SYNDROME 7, Light complexion, Early-onset retinal dystrophy, Leber congenital amaurosis 8, Retinitis pigmentosa 12, Ectodermal dysplasia 13, hair/tooth type, ECTODERMAL DYSPLASIA 14, HAIR/TOOTH TYPE WITH OR WITHOUT HYPOHIDROSIS, Ectodermal dysplasia 9, hair/nail type, Ectodermal dysplasia skin fragility syndrome, Ectodermal dysplasia, anhidrotic, with T-cell immunodeficiency, autosomal dominant, Ectodermal dysplasia/short stature syndrome, Ectodermal dysplasia-syndactyly syndrome 1, Ectopia lentis, isolated autosomal recessive, Ectopic ossification, Kyphosis, Myositis, Short stature, Ectrodactyly, ectodermal dysplasia, and cleft lip/palate syndrome 3, Rapp-Hodgkin ectodermal dysplasia syndrome, TP63-Related Spectrum Disorders, EHLERS-DANLOS SYNDROME, ARTHROCHALASIA TYPE, 2, Ehlers-Danlos syndrome, autosomal recessive, cardiac valvular form, Ehlers-Danlos syndrome, classic type, Osteogenesis imperfecta type I, Osteogenesis imperfecta type III, Osteogenesis imperfecta with normal sclerae, dominant form, Osteogenesis imperfecta, recessive perinatal lethal, Postmenopausal osteoporosis, Ehlers-Danlos syndrome, hydroxylysine-deficient, Ehlers-Danlos syndrome, musculocontractural type, Ehlers-Danlos syndrome, periodontal type, 2, Ehlers-Danlos syndrome, type 8, Ehlers-Danlos syndrome, procollagen proteinase deficient, Infantile cortical hyperostosis, Ehlers-Danlos syndrome, progeroid type, 2, Ehlers-Danlos syndrome, type 3, Ehlers-Danlos syndrome, type 4, POLYMICROGYRIA WITH OR WITHOUT VASCULAR-TYPE EHLERS-DANLOS SYNDROME, Thoracic aortic aneurysm and aortic dissection, Ehlers-Danlos syndrome, type vii, autosomal recessive, Eichsfeld type congenital muscular dystrophy, Elevated serum creatine phosphokinase, Elliptocytosis 1, Elliptocytosis 2, Hereditary pyropoikilocytosis, Prenatal anemia, Elliptocytosis 3, Emery-Dreifuss muscular dystrophy 1, X-linked, Emery-Dreifuss muscular dystrophy 4, autosomal dominant, Spinocerebellar ataxia, autosomal recessive 8, Emery-Dreifuss muscular dystrophy 6, EMG abnormality, Lower limb amyotrophy, Minicore myopathy, Talipes equinovarus, Enamel-renal syndrome, Encephalocele, Joubert syndrome, Joubert syndrome 9, Meckel syndrome type 6, Meckel-Gruber syndrome, Microcephaly, Narrow chest, Oligohydramnios, Encephalopathy due to defective mitochondrial and peroxisomal fission 1, Inborn genetic diseases, Optic atrophy 5, Encephalopathy, acute, infection-induced, 3, suceptibility to, ENCEPHALOPATHY, PROGRESSIVE, EARLY-ONSET, WITH BRAIN ATROPHY AND SPASTICITY, Encephalopathy, progressive, early-onset, with brain atrophy and thin corpus callosum, Encephalopathy, progressive, early-onset, with brain edema and/or leukoencephalopathy, Encephalopathy, progressive, with amyotrophy and optic atrophy, Endometrial carcinoma, Familial adenomatous polyposis 4, Endometrial neoplasm, Endplate acetylcholinesterase deficiency, Enlarged vestibular aqueduct, Pendred syndrome, Rare genetic deafness, Enterokinase deficiency, EPIDERMODYSPLASIA VERRUCIFORMIS, Epidermolysa bullosa simplex and limb girdle muscular dystrophy, Epidermolysis bullosa dystrophica, autosomal recessive, localisata variant, Epidermolysis bullosa herpetiformis, Dowling-Meara, Epidermolysis bullosa junctionalis with pyloric atresia, Epidermolysis bullosa simplex with migratory circinate erythema, Epidermolysis bullosa simplex with mottled pigmentation, Epidermolysis bullosa simplex with pyloric atresia, Epidermolysis bullosa simplex, autosomal recessive 2, Epilepsy, early-onset, vitamin b6-dependent, Epilepsy, familial adult myoclonic, 5, Epilepsy, familial focal, with variable foci 1, Rolandic epilepsy, Seizures, Epilepsy, familial focal, with variable foci 2, Epilepsy, familial focal, with variable foci 3, Epilepsy, familial temporal lobe, 7, Epilepsy, focal, with speech disorder and with or without mental retardation, Epilepsy, hearing loss, mental retardation syndrome, Epilepsy, idiopathic generalized, susceptibility to, 15; Inborn genetic diseases, Epilepsy, lateral temporal lobe, autosomal dominant, Epilepsy, nocturnal frontal lobe, 5, Epilepsy, nocturnal frontal lobe, type 3, Epilepsy, nocturnal frontal lobe, type 4, Epilepsy, progressive myoclonic 2b, Lafora disease, Epilepsy, progressive myoclonic 3, Epilepsy, progressive myoclonic 4, with or without renal failure, Epilepsy, progressive myoclonic 5, Sensory ataxic neuropathy, dysarthria, and ophthalmoparesis, Epilepsy, progressive myoclonic 6, Muscular dystrophy, GLUT1 deficiency syndrome 1, GLUT1 deficiency syndrome 1, autosomal recessive, Microcephaly, intellectual deficiency, Epileptic encephalopathy, Epileptic encephalopathy, childhood-onset, Epileptic encephalopathy, early infantile, 1, Epileptic encephalopathy, early infantile, 19, Epileptic encephalopathy, early infantile, 23, Epileptic encephalopathy, early infantile, 24, Epileptic encephalopathy, early infantile, 25, Epileptic encephalopathy, early infantile, 26, Epileptic encephalopathy, early infantile, 27, Mental retardation, autosomal dominant 6, Epileptic encephalopathy, early infantile, 28, Epileptic encephalopathy, early infantile, 30, Epileptic encephalopathy, early infantile, 31, Epileptic encephalopathy, early infantile, 32, Epileptic encephalopathy, early infantile, 33, Epileptic encephalopathy, early infantile, 42, Episodic ataxia type 2, Familial hemiplegic migraine type 1, Spinocerebellar ataxia 6, Epileptic encephalopathy, early infantile, 43, Epileptic encephalopathy, early infantile, 46, Epileptic encephalopathy, early infantile, 47, Epileptic encephalopathy, early infantile, 48, Epileptic encephalopathy, early infantile, 51, Infantile encephalopathy, Epileptic encephalopathy, early infantile, 53, Epileptic encephalopathy, early infantile, 54, Epileptic encephalopathy, early infantile, 58, Epileptic encephalopathy, early infantile, 68, Epileptic encephalopathy, early infantile, 69, Epileptic encephalopathy, early infantile, 70, Epileptic encephalopathy, early infantile, 73, Generalized epilepsy with febrile seizures plus 3, Rolandic epilepsy, Seizures, Epileptic encephalopathy, early infantile, 74, Epileptic encephalopathy, early infantile, 75, Epileptic encephalopathy, early infantile, 76, Epileptic encephalopathy, infantile or early childhood, 1, Epileptic encephalopathy, infantile or early childhood, 2, Epileptic encephalopathy, infantile or early childhood, 3, Epiphyseal dysplasia, multiple, with myopia and conductive deafness, Myopia, Episodic ataxia type 1, Episodic ataxia, type 6, Episodic pain syndrome, familial, 3, ERCC2-Related Disorders, Mixed Phenotype Acute Leukemia, T/Myeloid, Not Otherwise Specified, Erythema, Erythropoietic protoporphyria, Jaundice, Erythrocytosis 6, familial, HEMOGLOBIN CHEMILLY, HEMOGLOBIN HELSINKI, Erythroderma, congenital, with palmoplantar keratoderma, hypotrichosis, and hyper-ige, Erythroderma, ichthyosiform, congenital reticular, Erythrokeratodermia variabilis, Erythrokeratodermia variabilis et progressiva 2, Erythrokeratodermia variabilis et progressiva 4, Erythrokeratodermia variabilis et progressiva 5, Estrogen resistance, Ethylmalonic encephalopathy, Even-plus syndrome, Exfoliative ichthyosis, autosomal recessive, ichthyosis bullosa of siemens-like, Expressive language delay, Global developmental delay, NEURODEVELOPMENTAL DISORDER WITH DYSMORPHIC FACIES AND DISTAL LIMB ANOMALIES, Exudative retinopathy, Exudative vitreoretinopathy 1, Familial exudative vitreoretinopathy, Fabry disease, Fabry disease, cardiac variant, Factor V deficiency, Factor V Hong Kong, Factor VII deficiency, FACTOR VIII (OKAYAMA), Hereditary factor IX deficiency disease, Factor X deficiency, Failure of tooth eruption, primary, Familial acne inversa 1, Familial adenomatous polyposis 4, Familial amyloid nephropathy with urticaria AND deafness, Familial cold urticaria, Familial amyloid polyneuropathy, Iowa type, Familial atypical mycobacteriosis, type 1, X-linked, Familial cardiomyopathy, Familial cold autoinflammatory syndrome 2, Familial cold autoinflammatory syndrome 4, Familial dilated cardiomyopathy, Primary dilated cardiomyopathy, Familial febrile seizures 8, Familial hemiplegic migraine type 3, Familial hemophagocytic lymphohistiocytosis, Hemophagocytic lymphohistiocytosis, familial, 5, Familial hypercholesterolemia, Familial hyperinsulinism, Persistent hyperinsulinemic hypoglycemia of infancy, Familial hypertrophic cardiomyopathy 1, Familial hypertrophic cardiomyopathy 10, Familial hypertrophic cardiomyopathy 11, Familial hypertrophic cardiomyopathy 16, Familial hypertrophic cardiomyopathy 17, Familial hypertrophic cardiomyopathy 2, Familial restrictive cardiomyopathy 3, Left ventricular noncompaction 6, Primary dilated cardiomyopathy, Familial hypertrophic cardiomyopathy 20, Familial hypertrophic cardiomyopathy 3, Familial hypertrophic cardiomyopathy 4, Primary familial hypertrophic cardiomyopathy, Familial hypertrophic cardiomyopathy 6, Glycogen storage disease of heart, lethal congenital, Familial hypertrophic cardiomyopathy 7, Familial hypertrophic cardiomyopathy 8, Familial hypoalphalipoproteinemia, Familial hypobetalipoproteinemia, Familial hypocalciuric hypercalcemia, Hypocalcemia, autosomal dominant 1, Familial hypokalemia-hypomagnesemia, Familial hypoplastic, glomerulocystic kidney, Renal cysts and diabetes syndrome, Familial infantile myasthenia, Familial juvenile gout, Glomerulocystic kidney disease with hyperuricemia and isosthenuria, Medullary cystic kidney disease 2, Familial Mediterranean fever, Familial mediterranean fever, autosomal dominant, Familial partial lipodystrophy 2, Familial partial lipodystrophy 3, Familial platelet disorder with associated myeloid malignancy, Familial porphyria cutanea tarda, Familial progressive hyperpigmentation with or without hypopigmentation, Familial pulmonary capillary hemangiomatosis, Familial renal glucosuria, Familial renal hypouricemia, Hereditary renal hypouricemia, Familial type 3 hyperlipoproteinemia, Familial visceral amyloidosis, Ostertag type, Familial X-linked hypophosphatemic vitamin D refractory rickets, Fanconi anemia, Fanconi anemia, complementation group A, Neuroblastoma, Fanconi anemia, complementation group B, Fanconi anemia, complementation group D2, Fanconi anemia, complementation group G, Fanconi anemia, complementation group L, Fanconi anemia, complementation group P, Fanconi anemia, complementation group Q, Pre-B-cell acute lymphoblastic leukemia, Fanconi anemia, complementation group V, FANCONI ANEMIA, COMPLEMENTATION GROUP W, Fanconi-Bickel syndrome, Farber disease, Fatal familial insomnia, Genetic prion diseases, Huntington disease-like 1, Jakob-Creutzfeldt disease, Feeding difficulties, Laryngeal hypoplasia, Ventricular septal defect, FETAL AKINESIA DEFORMATION SEQUENCE 3, Fever-associated acute infantile liver failure syndrome, FGFR2 related craniosynostosis, Jackson-Weiss syndrome, Pfeiffer syndrome, Fibrochondrogenesis, FIBROMATOSIS, GINGIVAL, 5, Gingival fibromatosis 1, Fibrosis of extraocular muscles, congenital, 1, Fibrosis of extraocular muscles, congenital, 3b, Fibrosis of extraocular muscles, congenital, 3a, with or without extraocular involvement, Fibular hypoplasia and complex brachydactyly, Type A2 brachydactyly, Filippi syndrome, Finnish congenital nephrotic syndrome, Fleck corneal dystrophy, FLNA related lung disease, Floating-Harbor syndrome, Focal cortical dysplasia type II, Lymphangiomyomatosis, Tuberous sclerosis 1, Tuberous sclerosis syndrome, Focal segmental glomerulosclerosis 2, Focal segmental glomerulosclerosis 6, Follicle-stimulating hormone deficiency, isolated, Follicular lymphoma, Follicular thyroid carcinoma, Fontaine progeroid syndrome, Foveal hypoplasia 2, FOVEAL HYPOPLASIA 2 WITH OPTIC NERVE MISROUTING AND ANTERIOR SEGMENT DYSGENESIS, Fragile X syndrome, Frank Ter Haar syndrome, FRASER SYNDROME 2, Friedreich's ataxia, Frontometaphyseal dysplasia, Melnick-Needles syndrome, Oto-palato-digital syndrome, type II, Periventricular nodular heterotopia 1, Frontonasal dysplasia 2, Frontotemporal dementia, Frontotemporal dementia, ubiquitin-positive, Parkinson disease, late-onset, Parkinson-dementia syndrome, Pick's disease, Progressive supranuclear ophthalmoplegia, Fructose-biphosphatase deficiency, Fucosidosis, Fukuyama congenital muscular dystrophy, Fulminant hepatic failure, Fundus albipunctatus, autosomal recessive, Pigmentary retinal dystrophy, G6PD COSENZA, Glucose 6 phosphate dehydrogenase deficiency, G6PD MALAGA, G6PD SANTAMARIA, Galactosialidosis, late infantile, Galactosylceramide beta-galactosidase deficiency, Galloway-Mowat syndrome 1, GALLOWAY-MOWAT SYNDROME 2, X-LINKED, GALLOWAY-MOWAT SYNDROME 3, GALLOWAY-MOWAT SYNDROME 4, GALLOWAY-MOWAT SYNDROME 6, Gamma-aminobutyric acid transaminase deficiency, Gamma-glutamylcysteine synthetase deficiency, hemolytic anemia due to, Gastrointestinal stroma tumor, Polyps, multiple and recurrent inflammatory fibroid, gastrointestinal, GATA-1-related thrombocytopenia with dyserythropoiesis, Gaucher disease, Gaucher disease, atypical, due to saposin C deficiency, Gaucher disease, perinatal lethal, Gaucher's disease, type 1, Subacute neuronopathic Gaucher's disease, GBE1-Related Disorders, Glycogen storage disease IV, classic hepatic, Glycogen storage disease, type IV, Polyglucosan body disease, adult, Generalized arterial calcification of infancy 2, Pseudoxanthoma elasticum, Pseudoxanthoma elasticum, forme fruste, Generalized dominant dystrophic epidermolysis bullosa, Generalized epilepsy with febrile seizures plus, type 1, Severe myoclonic epilepsy in infancy, GENERALIZED EPILEPSY WITH FEBRILE SEIZURES PLUS, TYPE 10, Generalized epilepsy with febrile seizures plus, type 7, Hereditary sensory and autonomic neuropathy type IIA, Paroxysmal extreme pain disorder, Generalized epilepsy with febrile seizures plus, type 9, Generalized hypotonia, Gerstmann-Straussler-Scheinker syndrome, Gillespie syndrome, Gillessen-Kaesbach-Nishimura dysplasia, Gillessen-Kaesbach-Nishimura syndrome, Gingival fibromatosis 1, Inborn genetic diseases, Noonan syndrome, Noonan syndrome 4, Rasopathy, Glanzmann thrombasthenia, Glaucoma 1, open angle, a, digenic, Glaucoma 1, open angle, B, Glaucoma 1, open angle, F, Glaucoma 3, primary congenital, A, MYOC-Related Disorders, Primary open angle glaucoma juvenile onset 1, Glaucoma 3, primary congenital, d, Glaucoma, primary open angle, juvenile-onset, Glaucoma 3, primary congenital, E, GLB1-Related Disorders, GM1 gangliosidosis, Infantile GM1 gangliosidosis, Mucopolysaccharidosis, MPS-IV-B, Glioblastoma, Neoplasm of the breast, Renal cell carcinoma, papillary, 1, Smith-Kingsmore syndrome, Global developmental delay, absent or hypoplastic corpus callosum, and dysmorphic facies, Hydrocephalus, Seizures, Jaundice, Joubert syndrome 17, Orofaciodigital syndrome 6, Joubert syndrome 13, Typical Joubert syndrome MRI findings, Joubert syndrome 3, Neurodegeneration, Spastic paraplegia, Spastic paraplegia 56, autosomal recessive, Glomerulonephritis with sparse hair and telangiectases, Glomerulopathy with fibronectin deposits 2, Glomuvenous malformations, Glucocorticoid Deficiency, Glucocorticoid Deficiency 4 with Mineralocorticoid Deficiency, Glucocorticoid Deficiency 4 with or without Mineralocorticoid Deficiency, Glucocorticoid Deficiency with Achalasia, Glucocorticoid Resistance, Generalized, Glucose 6 Phosphate Dehydrogenase Deficiency, Glucose Transporter Type 1 Deficiency Syndrome, Glucose-6-Phosphate Transport Defect, Glycogen Storage Disease, Inborn Genetic Diseases, Phosphate Transport Defect, GLUT1 Deficiency Syndrome 1, GLUT1 Deficiency Syndrome 1, Autosomal Recessive, GLUT1 Deficiency Syndrome 2, Glutaric Acidemia IIA, Glutaric Aciduria, Type 2, Glutaric Aciduria, Type 1, Lipid Storage Myopathy Due to Flavin Adenine Dinucleotide Synthetase Deficiency, Glycine Encephalopathy with Normal Serum Glycine, Glycogen Storage Disease 0, Muscle, Glycogen Storage Disease II, Adult Form, Glycogen Storage Disease, Type II, Glycogen Storage Disease IIIa, Glycogen Storage Disease Type III, Glycogen Storage Disease IIIb, Glycogen Storage Disease IV, Classic Hepatic, Glycogen Storage Disease, Type IV, Glycogen Storage Disease IV, Congenital Neuromuscular, Glycogen Storage Disease IV, Fatal Perinatal Neuromuscular, Glycogen Storage Disease IXa2, Glycogen Storage Disease IXc, Glycogen Storage Disease IXd, Glycogen Storage Disease of Heart, Lethal Congenital, Glycogen Storage Disease Type 1A, GM1 Gangliosidosis Type 2, Gangliosidosis GM1 Type 3, Infantile GM1 Gangliosidosis, Mucopolysaccharidosis, MPS-IV-B, Gm2-Gangliosidosis, Adult, Tay-Sachs Disease, Gm2-Gangliosidosis, Subacute, Gnathodiaphyseal Dysplasia, Limb-Girdle Muscular Dystrophy, Type 2L, Miyoshi Muscular Dystrophy 3, GNE Myopathy, Sialuria, GNPTAB-Related Disorders, I Cell Disease, Pseudo-Hurler Polydystrophy, Gonadotropin-Independent Familial Sexual Precocity, Leydig Cell Agenesis, Precocious Puberty in Males, Gordon's Syndrome, Oculomelic Amyoplasia, Gorlin Syndrome, Hereditary Cancer-Predisposing Syndrome, Gray Platelet Syndrome, Greenberg Dysplasia, Greig Cephalopolysyndactyly Syndrome, Pallister-Hall Syndrome, Griscelli Disease, Griscelli Syndrome Type 2, Growth and Mental Retardation, Mandibulofacial Dysostosis, Microcephaly, and Cleft Palate, Growth Retardation, Intellectual Developmental Disorder, Hypotonia, and Hepatopathy, Gyrate Atrophy, Ornithine Aminotransferase Deficiency, HADHA-Related Disorders, Long-Chain 3-Hydroxyacyl-CoA Dehydrogenase Deficiency, Haemorrhagic Telangiectasia 1, Hereditary Hemorrhagic Telangiectasia, Osler Hemorrhagic Telangiectasia Syndrome, Haim-Munk Syndrome, Hair-Pulling, Tourette Syndrome, Hajdu-Cheney Syndrome, Hand Foot Uterus Syndrome, Hartsfield Syndrome, Hay-Wells Syndrome of Ectodermal Dysplasia, Hb Niigata, Beta Thalassemia, Beta\hat{ }0\hat{ } Thalassemia, Hearing Impairment, Hearing Loss, Hystrix-Like Ichthyosis with Deafness, Keratitis-Ichthyosis-Deafness Syndrome, Autosomal Dominant, Mutilating Keratoderma, Heart Block, Nonprogressive, Heart-Hand Syndrome, Slovenian Type, Heimler Syndrome 2, Peroxisome Biogenesis Disorder 4B, Peroxisome Biogenesis Disorder 4a (Zellweger), Heinz Body Anemia, Hemochromatosis Type 1, Hemochromatosis Type 2A, Hemochromatosis Type 3, Hemochromatosis Type 4, Hemoglobinopathy, Hemolytic Anemia, Hemolytic Anemia Due to Hexokinase Deficiency, Hemolytic Disease of Fetus or Newborn Due to Isoimmunization, Hemophagocytic Lymphohistiocytosis, Familial, 2, Stuve-Wiedemann Syndrome, Hemophagocytic Lymphohistiocytosis, Familial, 3, Hemorrhagic Destruction of the Brain, Subependymal Calcification, and Cataracts, Hennekam Lymphangiectasia-Lymphedema Syndrome, Hennekam Lymphangiectasia-Lymphedema Syndrome 2, Hennekam Lymphangiectasia-Lymphedema Syndrome 3, Heparin Cofactor II Deficiency, Hepatic Failure, Early-Onset, and Neurologic Disorder Due to Cytochrome C Oxidase Deficiency, Hepatic Methionine Adenosyltransferase Deficiency, Hepatic Venoocclusive Disease with Immunodeficiency, Hepatoerythropoietic Porphyria, Hereditary Acrodermatitis Enteropathica, Hereditary Angioedema Type 1, Hereditary Angioneurotic Edema, Hereditary C1 Esterase Inhibitor Deficiency-Dysfunctional Factor, Hereditary Cancer-Predisposing Syndrome, Hereditary Cerebral Amyloid Angiopathy, Icelandic Type, Hereditary Diffuse Leukoencephalopathy with Spheroids, Hereditary Factor IX Deficiency Disease, Hereditary Factor VIII Deficiency Disease, Hereditary Fructosuria, Hereditary Hemochromatosis, Hereditary Hemorrhagic Telangiectasia Type 2, Hereditary Hypotrichosis Simplex, Hypotrichosis 7, Woolly Hair, Autosomal Recessive 2, with or without Hypotrichosis, Hereditary Insensitivity to Pain with Anhidrosis, Hereditary Lymphedema, Hereditary Nephrotic Syndrome, Nephrotic Syndrome, Idiopathic, Steroid-Resistant, Hereditary Pancreatitis, Trypsinogen Deficiency, Hereditary Persistence of Fetal Hemoglobin, KLF1-Related, Hereditary Pyropoikilocytosis, Hereditary Sensory and Autonomic Neuropathy Type IC, Hereditary Sensory and Autonomic Neuropathy Type IIA, Mental Retardation, Autosomal Dominant 9, Spastic Paraplegia 30, Autosomal Recessive, Hereditary Sensory Neuropathy Type 1 D, Spastic Paraplegia 3, Hereditary Sensory Neuropathy Type IE, Hereditary Sideroblastic Anemia, Hereditary Spastic Paraplegia, Spastic Paraplegia 7, Hermansky Pudlak Syndrome 2, Hermansky-Pudlak Syndrome, Hermansky-Pudlak Syndrome 1, Hermansky-Pudlak Syndrome 3, Hermansky-Pudlak Syndrome 4, Hermansky-Pudlak Syndrome 5, Hermansky-Pudlak Syndrome 6, Hermansky-Pudlak Syndrome 8, Heterotaxy, Visceral, 2, Autosomal, Heterotaxy, Visceral, 8, Autosomal, Situs Inversus Totalis, Heterotaxy, Visceral, X-Linked, Heterotopia, Heterotopia, Periventricular Nodular, X-Linked Dominant, with Melnick-Needles Syndrome, Hexa, Dn Allele, Hirschsprung Disease 2, Waardenburg Syndrome Type 4A, Histiocytic Medullary Reticulosis, Severe Combined Immunodeficiency with Sensitivity to Ionizing Radiation, Severe Combined Immunodeficiency, B Cell-Negative, Severe Immunodeficiency, Autosomal Recessive, T-Cell Negative, B-Cell Negative, NK Cell-Positive, Histiocytosis-Lymphadenopathy Plus Syndrome, History of Neurodevelopmental Disorder, Mowat-Wilson Syndrome, Holocarboxylase Synthetase Deficiency, Holoprosencephaly 11, Holoprosencephaly 2, Holoprosencephaly 3, Holoprosencephaly 5, Holoprosencephaly 7, Holoprosencephaly 9, Holt-Oram Syndrome, Homocysteinemia Due to MTHFR Deficiency, Homocystinuria Due to CBS Deficiency, Homocystinuria, Pyridoxine-Responsive, Hutchinson-Gilford Progeria Syndrome, Atypical, Hutchinson-Gilford Progeria Syndrome, Childhood-Onset, Right Ventricular Cardiomyopathy, Hutchinson-Gilford Syndrome, Hyaline Fibromatosis Syndrome, Hydatidiform Mole, Hydatidiform Mole, Recurrent, 2, Hydranencephaly with Renal Aplasia-Dysplasia, Hydrocephalus Due to Aqueductal Stenosis, Spastic Paraplegia, X-Linked Hydrocephalus Syndrome, Hydrolethalus Syndrome, Hydrolethalus Syndrome 1, Hydroxykynureninuria, Hyperaldosteronism, Familial, Type II, Hyperammonemia, Type III, Hyperapobetalipoproteinemia, Hypercalcemia, Infantile, 2, Hypercalciuria, Childhood, Self-Limiting, Hyperchlorhidrosis, Isolated, Hypercholesterolemia, Autosomal Dominant, 3, Hypercholesterolemia, Autosomal Dominant, Type B, Hypercholesterolemia, Autosomal Recessive, Hyperekplexia 2, Hyperekplexia 3, Hyperekplexia Hereditary, Hyper-IgE Syndrome, Hyperimmunoglobulin E Syndrome, Hyperimmunoglobulin D with Periodic Fever, Mevalonic Aciduria, Porokeratosis, Disseminated Superficial Actinic 1, Hyperinsulinemic Hypoglycemia Familial 3, Hyperinsulinemic Hypoglycemia, Familial, 4, Hyperinsulinism-Hyperammonemia Syndrome, Hyperkalemic Periodic Paralysis Type 1, Hypokalemic Periodic Paralysis 1, Hypokalemic Periodic Paralysis, Type 2, Normokalemic Periodic Paralysis, Potassium-Sensitive, Paramyotonia Congenita of Von Eulenburg, Potassium Aggravated Myotonia, SCN4A-Related Disorder, Hyperlipoproteinemia, Type I, Hyperlipoproteinemia, Type ID, Hyperlysinemia, Hypermanganesemia with Dystonia 1, Hypermanganesemia with Dystonia 2, Hypermethioninemia Due to Adenosine Kinase Deficiency, Hyperornithinemia-Hyperammonemia-Homocitrullinuria Syndrome, Hyperostosis Cranialis Interna, Hyperphenylalaninemia, Non-PKU, Marfanoid Habitus and Intellectual Disability, Phenylketonuria, Hyperphosphatasemia with Bone Disease, Hyperphosphatasia with Mental Retardation Syndrome 1, Hyperphosphatasia with Mental Retardation Syndrome 4, Hyperphosphatasia with Mental Retardation Syndrome 5, Hyperproinsulinemia, Hypertelorism, Teebi Type, Opitz G/BBB Syndrome, Hypertension, Multiple Renal Cysts, Polycystic Kidney Disease, Adult Type, Hypertrophic Cardiomyopathy, Myopathy, Distal, 1, Primary Familial Hypertrophic Cardiomyopathy, Hyperuricemic Nephropathy, Familial Juvenile, 2, Hyperuricemic Nephropathy, Familial Juvenile, 4, Hypobetalipoproteinemia, Hypobetalipoproteinemia, Normotriglyceridemic, Hypocalcemia, Autosomal Dominant 1, Hypocalcemia, Autosomal Dominant 1, with Bartter Syndrome, Hypocalciuric Hypercalcemia, Familial, Type 1, Neonatal Severe Hyperparathyroidism, Hypocalciuric Hypercalcemia, Familial, Type II, Hypocalciuric Hypercalcemia, Familial, Type III, Hypocholesterolemia, Hypodysfibrinogenemia, Hypofibrinogenemia, Hypoglycemia with deficiency of glycogen synthetase in the liver, Hypogonadism, diabetes mellitus, alopecia, mental retardation and electrocardiographic abnormalities, Hypogonadotropic hypogonadism 10 with or without anosmia, Hypogonadotropic hypogonadism 10 without anosmia, Hypogonadotropic hypogonadism 12 with or without anosmia, Hypogonadotropic hypogonadism 21 with or without anosmia, Hypogonadotropic hypogonadism 22 with anosmia, Hypogonadotropic hypogonadism 4 with or without anosmia, Hypogonadotropic hypogonadism 7 with or without anosmia, Hypohidrotic ectodermal dysplasia with immune deficiency, Incontinentia pigmenti, atypical, Hypohidrotic X-linked ectodermal dysplasia, Tooth agenesis, selective, X-linked, 1, Hypokalemic periodic paralysis 1, Malignant hyperthermia susceptibility 5, Hypomagnesemia 1, intestinal, Hypomagnesemia 5, renal, with ocular involvement, HYPOMAGNESEMIA, SEIZURES, AND MENTAL RETARDATION 2, Hypomyelinating leukodystrophy 7, Hypomyelinating leukodystrophy 8, with or without oligodontia and/or hypogonadotropic hypogonadism, Hypomyelination and Congenital Cataract, Hypomyelination with brainstem and spinal cord involvement and leg spasticity, Hypoparathyroidism familial isolated, Hypoparathyroidism retardation dysmorphism syndrome, Kenny-Caffey syndrome type 1, Hypophosphatasia, perinatal lethal, Infantile hypophosphatasia, Hypophosphatemic rickets, autosomal recessive, 2, Hypoplastic left heart syndrome, Hypoplastic right heart syndrome, Wolff-Parkinson-White pattern, Hypoprebetalipoproteinemia, acanthocytosis, retinitis pigmentosa, and pallidal degeneration, Neurodegeneration with brain iron accumulation 1, atypical, Pigmentary pallidal degeneration, Hypospadias 1, X-linked, Hypospadias 2, X-linked, Hypothyroidism, central, and testicular enlargement, Hypothyroidism, congenital, nongoitrous, 1, Hypotonia, ataxia, and delayed development syndrome, Neurodevelopmental disorder, HYPOTONIA, ATAXIA, DEVELOPMENTAL DELAY, AND TOOTH ENAMEL DEFECT SYNDROME, Hypotonia, infantile, with psychomotor retardation and characteristic facies 2, Hypotonia, infantile, with psychomotor retardation and characteristic facies 3, Syndromic Infantile Encephalopathy, Hypotrichosis 13, HYPOTRICHOSIS 14, Hypotrichosis 4, Hypotrichosis simplex, Hypotrichosis-lymphedema-telangiectasia syndrome, I cell disease, Ichthyosis bullosa of Siemens, Ichthyosis vulgaris, Ichthyosis, congenital, autosomal recessive 11, Ichthyosis, congenital, autosomal recessive 12, Ichthyosis, cyclic, with epidermolytic hyperkeratosis, Ichthyosis, leukocyte vacuoles, alopecia, and sclerosing cholangitis, Ichthyosis, spastic quadriplegia, and mental retardation, Idiopathic basal ganglia calcification 1, Idiopathic basal ganglia calcification 5, Idiopathic fibrosing alveolitis, chronic form, Pulmonary fibrosis and/or bone marrow failure, telomere-related, 1, Idiopathic hypercalcemia of infancy, Muscle cramps, Idiopathic livedo reticularis with systemic involvement, Polyarteritis nodosa, childhoood-onset, IFAP syndrome with or without BRESHECK syndrome, IL21R immunodeficiency, Immune dysfunction with T-cell inactivation due to calcium entry defect 1, Immune dysfunction with T-cell inactivation due to calcium entry defect 2, Myopathy with tubular aggregates, Stormorken syndrome, Immune dysregulation-inflammatory bowel disease-arthritis-recurrent infections syndrome, Primary immunodeficiency, Immunodeficiency 14, Immunodeficiency 15, Immunodeficiency 18, severe combined immunodeficiency variant, Immunodeficiency 23, Immunodeficiency 24, Immunodeficiency 26 with or without neurologic abnormalities, Immunodeficiency 27b, Immunodeficiency 28, Immunodeficiency 29, Immunodeficiency 30, Immunodeficiency 31C, Immunodeficiency 36, Immunodeficiency 38 with basal ganglia calcification, Immunodeficiency 39, Immunodeficiency 45, Immunodeficiency 47, Immunodeficiency 51, Immunodeficiency 52, IMMUNODEFICIENCY 55, IMMUNODEFICIENCY 58, Immunodeficiency due to defect in cd3-zeta, Immunodeficiency due to ficolin 3 deficiency, Immunodeficiency with hyper IgM type 1, Immunodeficiency with hyper IgM type 2, Immunodeficiency with hyper IgM type 3, Immunodeficiency, common variable, 12, Immunodeficiency, common variable, 13, Immunodeficiency, X-Linked, with magnesium defect, Epstein-Barr virus infection, and neoplasia, Immunodeficiency-centromeric instability-facial anomalies syndrome 2, Immunodeficiency-centromeric instability-facial anomalies syndrome 3, Immunodeficiency-centromeric instability-facial anomalies syndrome 4, Inborn genetic diseases, Ichthyosis, cyclic, with epidermolytic hyperkeratosis, Ichthyosis, leukocyte vacuoles, alopecia, and sclerosing cholangitis, Ichthyosis, spastic quadriplegia, and mental retardation, Idiopathic basal ganglia calcification 1, Idiopathic basal ganglia calcification 5, Idiopathic fibrosing alveolitis, chronic form, Pulmonary fibrosis and/or bone marrow failure, telomere-related, 1, Idiopathic hypercalcemia of infancy, Muscle cramps, Idiopathic livedo reticularis with systemic involvement, Polyarteritis nodosa, childhoood-onset, IFAP syndrome with or without BRESHECK syndrome, IL21R immunodeficiency, Immune dysfunction with T-cell inactivation due to calcium entry defect 1, Immune dysfunction with T-cell inactivation due to calcium entry defect 2, Myopathy with tubular aggregates, Stormorken syndrome, Immune dysregulation-inflammatory bowel disease-arthritis-recurrent infections syndrome, Primary immunodeficiency, Immunodeficiency 14, Immunodeficiency 15, Immunodeficiency 18, severe combined immunodeficiency variant, Immunodeficiency 23, Immunodeficiency 24, Immunodeficiency 26 with or without neurologic abnormalities, Immunodeficiency 27b, Immunodeficiency 28, Immunodeficiency 29, Immunodeficiency 30, Immunodeficiency 31C, Immunodeficiency 36, Immunodeficiency 38 with basal ganglia calcification, Immunodeficiency 39, Immunodeficiency 45, Immunodeficiency 47, Immunodeficiency 51, Immunodeficiency 52, IMMUNODEFICIENCY 55, IMMUNODEFICIENCY 58, Immunodeficiency due to defect in cd3-zeta, Immunodeficiency due to ficolin 3 deficiency, Immunodeficiency with hyper IgM type 1, Immunodeficiency with hyper IgM type 2, Immunodeficiency with hyper IgM type 3, Immunodeficiency, common variable, 12, Immunodeficiency, common variable, 13, Immunodeficiency, X-Linked, with magnesium defect, Epstein-Barr virus infection, and neoplasia, Immunodeficiency-centromeric instability-facial anomalies syndrome 2, Immunodeficiency-centromeric instability-facial anomalies syndrome 3, Immunodeficiency-centromeric instability-facial anomalies syndrome 4, Inborn genetic diseases, Ichthyosis, cyclic, with epidermolytic hyperkeratosis, Ichthyosis, leukocyte vacuoles, alopecia, and sclerosing cholangitis, Ichthyosis, spastic quadriplegia, and mental retardation, Idiopathic basal ganglia calcification 1, Idiopathic basal ganglia calcification 5, Idiopathic fibrosing alveolitis, chronic form, Pulmonary fibrosis and/or bone marrow failure, telomere-related, 1, Idiopathic hypercalcemia of infancy, Muscle cramps, Idiopathic livedo reticularis with systemic involvement, Polyarteritis nodosa, childhoood-onset, IFAP syndrome with or without BRESHECK syndrome, IL21R immunodeficiency, Immune dysfunction with T-cell inactivation due to calcium entry defect 1, Immune dysfunction with T-cell inactivation due to calcium entry defect 2, Myopathy with tubular aggregates, Stormorken syndrome, Immune dysregulation-inflammatory bowel disease-arthritis-recurrent infections syndrome, Primary immunodeficiency, Immunodeficiency 14, Immunodeficiency 15, Immunodeficiency 18, severe combined immunodeficiency variant, Immunodeficiency 23, Immunodeficiency 24, Immunodeficiency 26 with or without neurologic abnormalities, Immunodeficiency 27b, Immunodeficiency 28, Immunodeficiency 29, Immunodeficiency 30, Immunodeficiency 31C, Immunodeficiency 36, Immunodeficiency 38 with basal ganglia calcification, Immunodeficiency 39, Immunodeficiency 45, Immunodeficiency 47, Immunodeficiency 51, Immunodeficiency 52, IMMUNODEFICIENCY 55, IMMUNODEFICIENCY 58, Immunodeficiency due to defect in cd3-zeta, Immunodeficiency due to ficolin 3 deficiency, Immunodeficiency with hyper IgM type 1, Immunodeficiency with hyper IgM type 2, Immunodeficiency with hyper IgM type 3, Immunodeficiency, common variable, 12, Immunodeficiency, common variable, 13, Immunodeficiency, X-Linked, with magnesium defect, Epstein-Barr virus infection, and neoplasia, Immunodeficiency-centromeric instability-facial anomalies syndrome 2, Immunodeficiency-centromeric instability-facial anomalies syndrome 3, Immunodeficiency-centromeric instability-facial anomalies syndrome 4, Inborn genetic diseases, Ichthyosis, cyclic, with epidermolytic hyperkeratosis, Ichthyosis, leukocyte vacuoles, alopecia, and sclerosing cholangitis, Ichthyosis, spastic quadriplegia, and mental retardation, Idiopathic basal ganglia calcification 1, Idiopathic basal ganglia calcification 5, Idiopathic fibrosing alveolitis, chronic form, Pulmonary fibrosis and/or bone marrow failure, telomere-related, 1, Idiopathic hypercalcemia of infancy, Muscle cramps, Idiopathic livedo reticularis with systemic involvement, Polyarteritis nodosa, childhoood-onset, IFAP syndrome with or without BRESHECK syndrome, IL21R immunodeficiency, Immune dysfunction with T-cell inactivation due to calcium entry defect 1, Immune dysfunction with T-cell inactivation due to calcium entry defect 2, Myopathy with tubular aggregates, Stormorken syndrome, Immune dysregulation-inflammatory bowel disease-arthritis-recurrent infections syndrome, Primary immunodeficiency, Immunodeficiency 14, Immunodeficiency 15, Immunodeficiency 18, severe combined immunodeficiency variant, Immunodeficiency 23, Immunodeficiency 24, Immunodeficiency 26 with or without neurologic abnormalities, Immunodeficiency 27b, Immunodeficiency 28, Immunodeficiency 29, Immunodeficiency 30, Immunodeficiency 31C, Immunodeficiency 36, Immunodeficiency 38 with basal ganglia calcification, Immunodeficiency 39, Immunodeficiency 45, Immunodeficiency 47, Immunodeficiency 51, Immunodeficiency 52, IMMUNODEFICIENCY 55, IMMUNODEFICIENCY 58, Immunodeficiency due to defect in cd3-zeta, Immunodeficiency due to ficolin 3 deficiency, Immunodeficiency with hyper IgM type 1, Immunodeficiency with hyper IgM type 2, Immunodeficiency with hyper IgM type 3, Immunodeficiency, common variable, 12, Immunodeficiency, common variable, 13, Immunodeficiency, X-Linked, with magnesium defect, Epstein-Barr virus infection, and neoplasia, Immunodeficiency-centromeric instability-facial anomalies syndrome 2, Immunodeficiency-centromeric instability-facial anomalies syndrome 3, Immunodeficiency-centromeric instability-facial anomalies syndrome 4, Inborn genetic diseases, Ichthyosis, cyclic, with epidermolytic hyperkeratosis, Ichthyosis, leukocyte vacuoles, alopecia, and sclerosing cholangitis, Ichthyosis, spastic quadriplegia, and mental retardation, Idiopathic basal ganglia calcification 1, Idiopathic basal ganglia calcification 5, Idiopathic fibrosing alveolitis, chronic form, Pulmonary fibrosis and/or bone marrow failure, telomere-related, 1, Idiopathic hypercalcemia of infancy, Muscle cramps, Idiopathic livedo reticularis with systemic involvement, Polyarteritis nodosa, childhoood-onset, IFAP syndrome with or without BRESHECK syndrome, IL21R immunodeficiency, Immune dysfunction with T-cell inactivation due to calcium entry defect 1, Immune dysfunction with T-cell inactivation due to calcium entry defect 2, Myopathy with tubular aggregates, Stormorken syndrome, Immune dysregulation-inflammatory bowel disease-arthritis-recurrent infections syndrome, Primary immunodeficiency, Immunodeficiency 14, Immunodeficiency 15, Immunodeficiency 18, severe combined immunodeficiency variant, Immunodeficiency 23, Immunodeficiency 24, Immunodeficiency 26 with or without neurologic abnormalities, Immunodeficiency 27b, Immunodeficiency 28, Immunodeficiency 29, Immunodeficiency 30, Immunodeficiency 31C, Immunodeficiency 36, Immunodeficiency 38 with basal ganglia calcification, Immunodeficiency 39, Immunodeficiency 45, Immunodeficiency 47, Immunodeficiency 51, Immunodeficiency 52, IMMUNODEFICIENCY 55, IMMUNODEFICIENCY 58, Immunodeficiency due to defect in cd3-zeta, Immunodeficiency due to ficolin 3 deficiency, Immunodeficiency with hyper IgM type 1, Immunodeficiency with hyper IgM type 2, Immunodeficiency with hyper IgM type 3, Immunodeficiency, common variable, 12, Immunodeficiency, common variable, 13, Immunodeficiency, X-Linked, with magnesium defect, Epstein-Barr virus infection, and neoplasia, Immunodeficiency-centromeric instability-facial anomalies syndrome 2, Immunodeficiency-centromeric instability-facial anomalies syndrome 3, Immunodeficiency-centromeric instability-facial anomalies syndrome 4, Inborn genetic diseases, Ichthyosis, cyclic, with epidermolytic hyperkeratosis, Ichthyosis, leukocyte vacuoles, alopecia, and sclerosing cholangitis, Ichthyosis, spastic quadriplegia, and mental retardation, Idiopathic basal ganglia calcification 1, Idiopathic basal ganglia calcification 5, Idiopathic fibrosing alveolitis, chronic form, Pulmonary fibrosis and/or bone marrow failure, telomere-related, 1, Idiopathic hypercalcemia of infancy, Muscle cramps, Idiopathic livedo reticularis with systemic involvement, Polyarteritis nodosa, childhoood-onset, IFAP syndrome with or without BRESHECK syndrome, IL21R immunodeficiency, Immune dysfunction with T-cell inactivation due to calcium entry defect 1, Immune dysfunction with T-cell inactivation due to calcium entry defect 2, Myopathy with tubular aggregates, Stormorken syndrome, Immune dysregulation-inflammatory bowel disease-arthritis-recurrent infections syndrome, Primary immunodeficiency, Immunodeficiency 14, Immunodeficiency 15, Immunodeficiency 18, severe combined immunodeficiency variant, Immunodeficiency 23, Immunodeficiency 24, Immunodeficiency 26 with or without neurologic abnormalities, Immunodeficiency 27b, Immunodeficiency 28, Immunodeficiency 29, Immunodeficiency 30, Immunodeficiency 31C, Immunodeficiency 36, Immunodeficiency 38 with basal ganglia calcification, Immunodeficiency 39, Immunodeficiency 45, Immunodeficiency 47, Immunodeficiency 51, Immunodeficiency 52, IMMUNODEFICIENCY 55, IMMUNODEFICIENCY 58, Immunodeficiency due to defect in cd3-zeta, Immunodeficiency due to ficolin 3 deficiency, Immunodeficiency with hyper IgM type 1, Immunodeficiency with hyper IgM type 2, Immunodeficiency with hyper IgM type 3, Immunodeficiency, common variable, 12, Immunodeficiency, common variable, 13, Immunodeficiency, X-Linked, with magnesium defect, Epstein-Barr virus infection, and neoplasia, Immunodeficiency-centromeric instability-facial anomalies syndrome 2, Immunodeficiency-centromeric instability-facial anomalies syndrome 3, Immunodeficiency-centromeric instability-facial anomalies syndrome 4, Inborn genetic diseases, Ichthyosis, cyclic, with epidermolytic hyperkeratosis, Ichthyosis, leukocyte vacuoles, alopecia, and sclerosing cholangitis, Ichthyosis, spastic quadriplegia, and mental retardation, Idiopathic basal ganglia calcification 1, Idiopathic basal ganglia calcification 5, Idiopathic fibrosing alveolitis, chronic form, Pulmonary fibrosis and/or bone marrow failure, telomere-related, 1, Idiopathic hypercalcemia of infancy, Muscle cramps, Idiopathic livedo reticularis with systemic involvement, Polyarteritis nodosa, childhoood-onset, IFAP syndrome with or without BRESHECK syndrome, IL21R immunodeficiency, Immune dysfunction with T-cell inactivation due to calcium entry defect 1, Immune dysfunction with T-cell inactivation due to calcium entry defect 2, Myopathy with tubular aggregates, Stormorken syndrome, Immune dysregulation-inflammatory bowel disease-arthritis-recurrent infections syndrome, Primary immunodeficiency, Immunodeficiency 14, Immunodeficiency 15, Immunodeficiency 18, severe combined immunodeficiency variant, Immunodeficiency 23, Immunodeficiency 24, Immunodeficiency 26 with or without neurologic abnormalities, Immunodeficiency 27b, Immunodeficiency 28, Immunodeficiency 29, Immunodeficiency 30, Immunodeficiency 31C, Immunodeficiency 36, Immunodeficiency 38 with basal ganglia calcification, Immunodeficiency 39, Immunodeficiency 45, Lethal congenital contracture syndrome 11, Lethal congenital contracture syndrome 5, Lethal congenital contracture syndrome 7, Lethal multiple pterygium syndrome, Multiple pterygium syndrome Escobar type, Lethal tight skin contracture syndrome, Leukemia, megakaryoblastic, of Down syndrome, Leukemia, Philadelphia chromosome-positive, resistant to imatinib, Leukocyte adhesion deficiency, Leukocyte adhesion deficiency type 1, Leukocyte adhesion deficiency, type III, Leukodystrophy, hypomyelinating, 10, Leukodystrophy, hypomyelinating, 11, Mandibulofacial dysostosis, Treacher Collins type, autosomal recessive, Leukodystrophy, hypomyelinating, 12, Leukoencephalopathy, LEUKODYSTROPHY, HYPOMYELINATING, 14, LEUKODYSTROPHY, HYPOMYELINATING, 15, LEUKODYSTROPHY, HYPOMYELINATING, 18, Leukodystrophy, hypomyelinating, 2, Spastic paraplegia, Leukodystrophy, hypomyelinating, 6, Metachromatic leukodystrophy, severe, Leukodystrophy, hypomyelinating, 9, Leukoencephalopathy with ataxia, Leukoencephalopathy with Brainstem and Spinal Cord Involvement and Lactate Elevation, Leukoencephalopathy with vanishing white matter, Ovarioleukodystrophy, LEUKOENCEPHALOPATHY, ACUTE REVERSIBLE, WITH INCREASED URINARY ALPHA-KETOGLUTARATE, Leukoencephalopathy, brain calcifications, and cysts, Leukoencephalopathy, progressive, with ovarian failure, Levy-Hollister syndrome, Leydig cell agenesis, Limb-girdle muscular dystrophy, type 1E, Limb-girdle muscular dystrophy, type 1F, Limb-girdle muscular dystrophy, type 2A, MUSCULAR DYSTROPHY, LIMB-GIRDLE, AUTOSOMAL DOMINANT 4, Limb-girdle muscular dystrophy, type 2B, Miyoshi muscular dystrophy 1, Proximal muscle weakness, Limb-girdle muscular dystrophy, type 2D, Muscular dystrophy, Sarcoglycanopathies, Limb-girdle muscular dystrophy, type 2E, Limb-girdle muscular dystrophy, type 2F, Limb-girdle muscular dystrophy, type 2G, Limb-girdle muscular dystrophy, type 2L, Limb-girdle muscular dystrophy, type 2Q, Limb-girdle muscular dystrophy-dystroglycanopathy, type C1, Limb-girdle muscular dystrophy-dystroglycanopathy, type C2, Limb-girdle muscular dystrophy-dystroglycanopathy, type C4, Muscular dystrophy-dystroglycanopathy (congenital with brain and eye anomalies), type A, 1, Limb-girdle muscular dystrophy-dystroglycanopathy, type C5, Walker-Warburg congenital muscular dystrophy, Linear skin defects with multiple congenital anomalies 2, Lipid proteinosis, Lipoyltransferase 1 deficiency, Lissencephaly 1, Lissencephaly 2, Lissencephaly 2, X-linked, Lissencephaly 3, Tubulinopathies, Lissencephaly 5, Lissencephaly 6, with microcephaly, Lissencephaly 8, Lissencephaly with decussation defect, Lissencephaly, X-linked, Subcortical laminar heterotopia, X-linked, Liver failure acute infantile, Localized epidermolytic hyperkeratosis, Loeys-Dietz syndrome 1, Loeys-Dietz syndrome 3, Loeys-Dietz syndrome 4, Long QT syndrome, Long QT syndrome 1, Long QT syndrome, LQT1 subtype, Romano-Ward syndrome, Long-chain 3-hydroxyacyl-CoA dehydrogenase deficiency, Mitochondrial trifunctional protein deficiency, Lopes-Maciel-Rodan syndrome, Lowe syndrome, Lower Urinary Tract Obstruction, Lung adenocarcinoma, Luscan-lumish syndrome, Lymphangiomyomatosis, Lymphedema, primary, with myelodysplasia, Lymphoproliferative syndrome 1, X-linked, X-Linked Lymphoproliferative Syndrome, Lymphoproliferative syndrome 2, X-linked, Lysinuric protein intolerance, Lysosomal acid lipase deficiency, Wolman disease, MacInnes syndrome, Macrocephaly, alopecia, cutis laxa, and scoliosis, Macrothrombocytopenia, Macrothrombocytopenia and granulocyte inclusions with or without nephritis or sensorineural hearing loss, Macrothrombocytopenia, familial, Bernard-Soulier type, Macular corneal dystrophy Type I, Macular corneal dystrophy, type II, Macular dystrophy, Macular dystrophy, vitelliform, adult-onset, Patterned dystrophy of retinal pigment epithelium, Retinitis pigmentosa, Retinitis pigmentosa 19, Stargardt disease 1, Majeed syndrome, Male infertility with teratozoospermia due to single gene mutation, Non-syndromic male infertility due to sperm motility disorder, SPERMATOGENIC FAILURE 33, SPERMATOGENIC FAILURE 38, Malformation of the heart and great vessels, Malignant hyperthermia, susceptibility to, 1, Minicore myopathy, Multi-minicore disease and atypical periodic paralysis, Myopathy, Central Core, Neuromuscular Diseases, RYR1-Related Disorders, Malignant neoplasm of body of uterus, Mental retardation, autosomal dominant 36, Uterine Carcinosarcoma, Malignant rhabdoid tumor, somatic, Malignant tumor of prostate, Malpuech facial clefting syndrome, Mandibuloacral dysostosis, Mandibuloacral dysplasia with type B lipodystrophy, Mandibulofacial dysostosis with alopecia, Maple syrup urine disease, Maple syrup urine disease type 1A, MAPLE SYRUP URINE DISEASE, CLASSIC, TYPE IB, Maple syrup urine disease, type 3, Marden Walker like syndrome, Marfan lipodystrophy syndrome, Marfan syndrome, Thoracic aortic aneurysm and aortic dissection, Marfan syndrome, neonatal, Marfan syndrome, severe classic, Marfan Syndrome/Loeys-Dietz Syndrome/Familial Thoracic Aortic Aneurysms and Dissections, Marshall/Stickler syndrome, Marshall-Smith syndrome, MARS-Related Disorder, Mast syndrome, Maturity onset diabetes mellitus in young, Maturity-onset diabetes of the young, type 3, Maturity-onset diabetes of the young, type 1, Maturity-onset diabetes of the young, type 2, Monogenic diabetes, Permanent neonatal diabetes mellitus, Transient neonatal diabetes mellitus 3, Maturity-onset diabetes of the young, type 13, McCune-Albright syndrome, PITUITARY ADENOMA 3, MULTIPLE TYPES, Meckel syndrome 13, Meckel syndrome type 1, Meckel syndrome type 3, Meckel syndrome type 6, Meckel syndrome type 8, Meckel syndrome, type 10, Meckel syndrome, type 11, Meckel-Gruber syndrome, Medium-chain acyl-coenzyme A dehydrogenase deficiency, Medulloblastoma, Medulloblastoma with extensive nodularity, Meesman's corneal dystrophy, Meester-loeys syndrome, Megalencephalic leukoencephalopathy with subcortical cysts 1, Megalencephaly cutis marmorata telangiectatica congenita, Megalencephaly-polymicrogyria-polydactyly-hydrocephalus syndrome 2, Megalencephaly-polymicrogyria-polydactyly-hydrocephalus syndrome 3, Megaloblastic anemia 1, Finnish type, Megaloblastic anemia due to dihydrofolate reductase deficiency, Megaloblastic anemia due to inborn errors of metabolism, Megalocornea, MEHMO syndrome, Meier-Gorlin syndrome 1, Meier-Gorlin syndrome 2, Meier-Gorlin syndrome 3, MEIER-GORLIN SYNDROME 8, Melnick-Fraser syndrome, Melnick-Needles syndrome, Oto-palato-digital syndrome, type II, Membranous cataract, MEND syndrome, Meningioma, Meningioma, familial, Neurofibromatosis, type 2, Menke-Hennekam syndrome 2, Menkes kinky-hair syndrome, Mental retardation 30, X-linked, Mental retardation 49, X-linked, Mental retardation and distinctive facial features with or without cardiac defects, Mental retardation and microcephaly with pontine and cerebellar hypoplasia, Mental retardation with language impairment and with or without autistic features, Mental retardation, autosomal dominant 1, Mental retardation, autosomal dominant 13, Mental retardation, autosomal dominant 14, Mental retardation, autosomal dominant 16, Mental retardation, autosomal dominant 18, Mental retardation, autosomal dominant 19, Mental retardation, autosomal dominant 21, Mental retardation, autosomal dominant 22, Mental retardation, autosomal dominant 23, Mental retardation, autosomal dominant 24, Mental retardation, autosomal dominant 26, Mental retardation, autosomal dominant 27, Mental retardation, autosomal dominant 30, Mental retardation, autosomal dominant 31, Mental retardation, autosomal dominant 32, Syndromic intellectual disability, Mental retardation, autosomal dominant 33, Mental retardation, autosomal dominant 35, Mental retardation, autosomal dominant 39, intellectual disability with severe speech impairment, Mental retardation, autosomal dominant 43, Mental retardation, autosomal dominant 44, MENTAL RETARDATION, AUTOSOMAL DOMINANT 46, MENTAL RETARDATION, AUTOSOMAL DOMINANT 48, Mental retardation, autosomal dominant 5, MENTAL RETARDATION, AUTOSOMAL DOMINANT 50, MENTAL RETARDATION, AUTOSOMAL DOMINANT 51, MENTAL RETARDATION, AUTOSOMAL DOMINANT 52, MENTAL RETARDATION, AUTOSOMAL DOMINANT 54, MENTAL RETARDATION, AUTOSOMAL DOMINANT 55, WITH SEIZURES, MENTAL RETARDATION, AUTOSOMAL DOMINANT 56, MENTAL RETARDATION, AUTOSOMAL DOMINANT 57, MENTAL RETARDATION, AUTOSOMAL DOMINANT 58, Mental retardation, autosomal dominant 6, Mental retardation, autosomal dominant 7, Mental retardation, autosomal dominant 9, Mental retardation, autosomal recessive 1, Mental retardation, autosomal recessive 12, Mental retardation, autosomal recessive 13, Mental retardation, autosomal recessive 15, Mental retardation, autosomal recessive 18, Mental retardation, autosomal recessive 27, Mental retardation, autosomal recessive 34, Mental retardation, autosomal recessive 36, Mental retardation, autosomal recessive 37, Mental retardation, autosomal recessive 38, Mental retardation, autosomal recessive 41, Mental retardation, autosomal recessive 44, Mental retardation, autosomal recessive 47, Mental retardation, autosomal recessive 53, Mental retardation, autosomal recessive 57, Mental retardation, autosomal recessive 58, Mental Retardation, Psychosocial, Mental retardation, stereotypic movements, epilepsy, and/or cerebral malformations, Mental retardation, syndromic 14, X-linked, Mental retardation, syndromic, Claes-Jensen type, X-linked, Spastic paraplegia, Mental retardation, X-linked 1, Severe intellectual deficiency, Mental retardation, X-linked 102, Mental retardation, X-linked 105, MENTAL RETARDATION, X-LINKED 106, Mental retardation, X-linked 12, Mental retardation, X-linked 18, Mental retardation, X-linked 19, Mental retardation, X-linked 61, Mental retardation, X-linked 72, Mental retardation, X-linked 93, Mental retardation, X-linked 96, Mental retardation, X-linked 98, Mental retardation, X-linked 99, Mental retardation, X-linked 99, syndromic, female-restricted, Mental retardation, X-linked, syndromic 13, Mental retardation, X-linked, syndromic 33, Mental retardation, X-linked, syndromic 34, Mental retardation, X-linked, syndromic, Raymond type, Mental retardation, X-linked, syndromic, Turner type, Mental retardation, X-linked, syndromic, wu type, Mental retardation-hypotonic facies syndrome X-linked, 1, Merosin deficient congenital muscular dystrophy, METABOLIC CRISES, RECURRENT, WITH RHABDOMYOLYSIS, CARDIAC ARRHYTHMIAS, AND NEURODEGENERATION, METABOLIC CRISES, RECURRENT, WITH VARIABLE ENCEPHALOMYOPATHIC FEATURES AND NEUROLOGIC REGRESSION, Metachondromatosis, Metachromatic leukodystrophy, Metachromatic leukodystrophy variant, Metaphyseal anadysplasia 2, Metaphyseal chondrodysplasia, Jansen type, Metaphyseal chondrodysplasia, Schmid type, Metatrophic dysplasia, Neuromuscular Diseases, Skeletal dysplasia, Methemoglobinemia type 2, Methemoglobinemia type 4, Methemoglobinemia, type I, METHYLCOBALAMIN DEFICIENCY, cbIG TYPE, Methylmalonic acidemia, Methylmalonic acidemia with homocystinuria, Methylmalonic acidemia with homocystinuria cbID, METHYLMALONIC ACIDURIA AND HOMOCYSTINURIA, cbIC TYPE, DIGENIC, METHYLMALONIC ACIDURIA AND HOMOCYSTINURIA, cbIJ TYPE, Methylmalonic aciduria cbIA type, Methylmalonic aciduria cbIB type, Methylmalonic aciduria due to methylmalonyl-CoA mutase deficiency, METHYLMALONIC ACIDURIA, mut(−) TYPE, METHYLMALONIC ACIDURIA, mut(0) TYPE, Methylmalonyl-CoA epimerase deficiency, Mevalonic aciduria, Michelin-tire baby, Microcephalic osteodysplastic primordial dwarfism type 2, Microcephaly 17, primary, autosomal recessive, MICROCEPHALY 20, PRIMARY, AUTOSOMAL RECESSIVE, MICROCEPHALY 22, PRIMARY, AUTOSOMAL RECESSIVE, Microcephaly and chorioretinopathy, autosomal recessive, 2, Microcephaly and chorioretinopathy, autosomal recessive, 3, Microcephaly with chorioretinopathy, autosomal recessive, Microcephaly with mental retardation and digital anomalies, Microcephaly with or without chorioretinopathy, lymphedema, or mental retardation, Microcephaly, epilepsy, and diabetes syndrome, MICROCEPHALY, SHORT STATURE, AND LIMB ABNORMALITIES, Microcephaly, short stature, and polymicrogyria with or without seizures, Microcephaly-capillary malformation syndrome, Microcytic anemia, Microphthalmia syndromic 3, Microphthalmia syndromic 5, Microphthalmia syndromic 9, Microphthalmia, cataracts, and iris abnormalities, Microphthalmia, isolated 2, Microphthalmia, isolated 4, Microphthalmia, isolated 5, Nanophthalmos 2, Microphthalmia, isolated, with coloboma 9, MICROPHTHALMIA, SYNDROMIC 15, Microphthalmia/coloboma and skeletal dysplasia syndrome, Midaortic syndrome, Midface hypoplasia, hearing impairment, elliptocytosis, and nephrocalcinosis, Mirror movements 1, Mirror movements 2, Mitchell-Riley syndrome, mitochondrial 3-hydroxy-3-methylglutaryl-CoA synthase deficiency, MITOCHONDRIAL COMPLEX I DEFICIENCY, NUCLEAR TYPE 10, MITOCHONDRIAL COMPLEX I DEFICIENCY, NUCLEAR TYPE 11, MITOCHONDRIAL COMPLEX I DEFICIENCY, NUCLEAR TYPE 16, MITOCHONDRIAL COMPLEX I DEFICIENCY, NUCLEAR TYPE 17, MITOCHONDRIAL COMPLEX I DEFICIENCY, NUCLEAR TYPE 33, Mitochondrial diseases, MITOCHONDRIAL COMPLEX I DEFICIENCY, NUCLEAR TYPE 5, MITOCHONDRIAL COMPLEX I DEFICIENCY, NUCLEAR TYPE 6, MITOCHONDRIAL COMPLEX I DEFICIENCY, NUCLEAR TYPE 9, Mitochondrial complex III deficiency, nuclear type 2, Mitochondrial complex III deficiency, nuclear type 9, MITOCHONDRIAL COMPLEX V (ATP SYNTHASE) DEFICIENCY, NUCLEAR TYPE 5, Mitochondrial diseases, Progressive external ophthalmoplegia with mitochondrial DNA deletions, autosomal recessive 5, Progressive sclerosing poliodystrophy, Spastic paraplegia 7, Mitochondrial DNA depletion syndrome 1 (MNGIE type), Mitochondrial DNA depletion syndrome 12a (cardiomyopathic type), autosomal dominant, Mitochondrial DNA depletion syndrome 13 (encephalomyopathic type), Mitochondrial DNA depletion syndrome 14 (cardioencephalomyopathic type), Mitochondrial DNAdepletion syndrome 2, Mitochondrial DNAdepletion syndrome 4B, MNGIE type, Mitochondrial DNA depletion syndrome 5 (encephalomyopathic with or without methylmalonic aciduria), Mitochondrial DNA depletion syndrome 7 (hepatocerebral type), Mitochondrial DNA depletion syndrome 9 (encephalomyopathic with methylmalonic aciduria), Mitochondrial DNA depletion syndrome, encephalomyopathic form, with renal tubulopathy, RRM2B-related mitochondrial disease, Mitochondrial DNA-depletion syndrome 3, hepatocerebral, Portal hypertension, noncirrhotic, Mitochondrial encephalopathy, Mitochondrial pyruvate carrier deficiency, Mitochondrial short-chain enoyl-coa hydratase 1 deficiency, Mitochondrial trifunctional protein deficiency, Miyoshi muscular dystrophy 1, Mohr-Tranebjaerg syndrome, Molybdenum cofactor deficiency, complementation group A, Molybdenum cofactor deficiency, complementation group C, Monocarboxylate transporter 1 deficiency, autosomal dominant, Monocarboxylate transporter 1 deficiency, autosomal recessive, Monogenic diabetes, Obesity, Morquio syndrome, Mucopolysaccharidosis, MPS-IV-A, Motor neuron disease, Mowat-Wilson syndrome, Moyamoya disease 6 with achalasia, Mucolipidosis III Gamma, Mucolipidosis type IV, Mucopolysaccharidosis type I, Mucopolysaccharidosis type VI, Mucopolysaccharidosis type VII, Mucopolysaccharidosis, MPS-II, Mucopolysaccharidosis, MPS-III-A, Sanfilippo syndrome, Mucopolysaccharidosis, MPS-III-B, Mucopolysaccharidosis, MPS-III-C, Retinitis pigmentosa 73, Mucopolysaccharidosis, MPS-III-D, Mucosa-associated lymphoma, Mulibrey nanism syndrome, Multicentric osteolysis nephropathy, Multicentric osteolysis, nodulosis and arthropathy, Multicystic renal dysplasia, bilateral, Multiple congenital anomalies-hypotonia-seizures syndrome 1, Multiple congenital anomalies-hypotonia-seizures syndrome 2, Multiple congenital anomalies-hypotonia-seizures syndrome 3, Multiple congenital exostosis, Multiple Cutaneous and Mucosal Venous Malformations, Multiple endocrine neoplasia, type 4, Multiple epiphyseal dysplasia 1, Multiple gastrointestinal atresias, Multiple mitochondrial dysfunctions syndrome 1, Multiple mitochondrial dysfunctions syndrome 3, Multiple mitochondrial dysfunctions syndrome 4, Multiple mitochondrial dysfunctions syndrome 6, Multiple pterygium syndrome Escobar type, Multiple sulfatase deficiency, Multiple synostoses syndrome 2, Symphalangism, proximal, 1 b, Muscle eye brain disease, Muscle weakness, Respiratory distress, Muscular Diseases, Muscular dystrophy, Muscular dystrophy, congenital, due to integrin alpha-7 deficiency, Muscular dystrophy, congenital, megaconial type, MUSCULAR DYSTROPHY, LIMB-GIRDLE, AUTOSOMAL RECESSIVE 23, Merosin deficient congenital muscular dystrophy, Muscular dystrophy, limb-girdle, type 2R, Myofibrillar myopathy 1, Scapuloperoneal syndrome, neurogenic, Kaeser type, Muscular dystrophy, limb-girdle, type 2y, Muscular dystrophy, limb-girdle, type 2z, Muscular dystrophy-dystroglycanopathy (congenital with brain and eye anomalies), type A, 1, Muscular dystrophy-dystroglycanopathy (congenital with brain and eye anomalies), type a, 10, Muscular dystrophy-dystroglycanopathy (congenital with brain and eye anomalies), type a, 12, Muscular dystrophy-dystroglycanopathy (congenital with mental retardation), type b, 14, Muscular dystrophy-dystroglycanopathy (limb-girdle), type c, 14, Muscular hypotonia, Nystagmus, Polydactyly, Myasthenia, limb-girdle, familial, Myasthenic syndrome, congenital, 11, associated with acetylcholine receptor deficiency, Myasthenic syndrome, congenital, 14, Myasthenic syndrome, congenital, 17, Sclerosteosis 2, Syndactyly Cenani Lenz type, Myasthenic syndrome, congenital, 18, Myasthenic syndrome, congenital, 19, Myasthenic syndrome, congenital, 22, Myasthenic syndrome, congenital, 3b, fast-channel, Myasthenic syndrome, congenital, 4a, slow-channel, Myasthenic syndrome, congenital, 7, presynaptic, Mycobacterial and viral infections, susceptibility to, autosomal recessive, Myelodysplasia, Myelofibrosis, Myeloperoxidase deficiency, MYH7-Related Disorders, Myopathy, distal, 1, Scapuloperoneal myopathy, MYH7-related, MYH9-related disorder, Macrothrombocytopenia and granulocyte inclusions with or without nephritis or sensorineural hearing loss, Myoclonic dystonia, Myoclonic epilepsy, familial infantile, Myoclonic-atonic epilepsy, Myoclonus, intractable, neonatal, Myofibrillar myopathy 3, Myoglobinuria, acute recurrent, autosomal recessive, Myokymia 1, Myopathy with extrapyramidal signs, Myopathy with postural muscle atrophy, X-linked, Myopathy with tubular aggregates, Myopathy, actin, congenital, with cores, Myopathy, areflexia, respiratory distress, and dysphagia, early-onset, Myopathy, Central Core, Myopathy, centronuclear, MYOPATHY, CENTRONUCLEAR, 6, WITH FIBER-TYPE DISPROPORTION, Myopathy, congenital, compton-north, MYOPATHY, CONGENITAL, WITH FAST-TWITCH (TYPE II) FIBER ATROPHY, Myopathy, congenital, with neuropathy and deafness, Myopathy, early-onset, with fatal cardiomyopathy, Myopathy, lactic acidosis, and sideroblastic anemia 2, MYOPATHY, MITOCHONDRIAL, AND ATAXIA, Myopathy, myofibrillar, 7, Myopathy, myofibrillar, 8, Myopathy, reducing body, X-linked, early-onset, severe, Myopathy, scapulohumeroperoneal, Myopathy, X-linked, with excessive autophagy, Myopia 22, autosomal dominant, Myopia 23, autosomal recessive, Myosin storage myopathy, Myotonia congenita, Naegeli-Franceschetti-Jadassohn syndrome, Nager syndrome, Nail disease, Nail disorder, nonsyndromic congenital, 10, Nail-patella syndrome, Nakajo syndrome, Nance-Horan syndrome, Nanophthalmos 4, Native American myopathy, Natural killer cell enteropathy, Navajo neurohepatopathy, Naxos disease, Nemaline myopathy 1, Nemaline myopathy 10, Nemaline myopathy 11, autosomal recessive, Nemaline myopathy 2, Nemaline myopathy 2, autosomal recessive, Nemaline myopathy 3, Nemaline myopathy 4, Nemaline myopathy 5, Neonatal adrenoleucodystrophy, Peroxisome biogenesis disorder 2A (Zellweger), Neonatal diabetes mellitus, Permanent neonatal diabetes mellitus, Neonatal insulin-dependent diabetes mellitus, Neonatal intrahepatic cholestasis caused by citrin deficiency, Neonatal pseudo-hydrocephalic progeroid syndrome, Neoplasm, Neoplasm of ovary, Noonan syndrome, Noonan syndrome 1, Noonan syndrome 10, Schwannomatosis 2, Short stature, Noonan syndrome 2, Noonan syndrome 3, Noonan syndrome 4, Noonan syndrome 5, Noonan syndrome 8, Noonan syndrome with multiple lentigines, Noonan syndrome-like disorder with juvenile myelomonocytic leukemia, Noonan syndrome-like disorder with loose anagen hair 2, Norum disease, Neuroblastoma, Neurodegeneration with brain iron accumulation 1, atypical, Neurodegeneration with brain iron accumulation 2b, Neurodegeneration with brain iron accumulation 4, Neurodegeneration with brain iron accumulation 5, NEURODEGENERATION WITH BRAIN IRON ACCUMULATION 7, NEURODEGENERATION, CHILDHOOD-ONSET, STRESS-INDUCED, WITH VARIABLE ATAXIA AND SEIZURES, NEURODEGENERATION, CHILDHOOD-ONSET, WITH CEREBELLAR ATROPHY, Neurodevelopmental abnormality, Neurodevelopmental delay, NEURODEVELOPMENTAL DISORDER WITH CENTRAL AND PERIPHERAL MOTOR DYSFUNCTION, NEURODEVELOPMENTAL DISORDER WITH CEREBELLAR ATROPHY AND WITH OR WITHOUT SEIZURES, Neurodevelopmental disorder with hypotonia, seizures, and absent language, NEURODEVELOPMENTAL DISORDER WITH MICROCEPHALY, SEIZURES, AND CORTICAL ATROPHY, Neurodevelopmental disorder with midbrain and hindbrain malformations, NEURODEVELOPMENTAL DISORDER WITH MOVEMENT ABNORMALITIES, ABNORMAL GAIT, AND AUTISTIC FEATURES, ZSWIM6 related intellectual disability, Neurodevelopmental disorder with or without anomalies of the brain, eye, or heart, Neurodevelopmental disorder with or without hyperkinetic movements and seizures, autosomal dominant, NEURODEVELOPMENTAL DISORDER WITH SEIZURES AND NONEPILEPTIC HYPERKINETIC MOVEMENTS, NEURODEVELOPMENTAL DISORDER, MITOCHONDRIAL, WITH ABNORMAL MOVEMENTS AND LACTIC ACIDOSIS, WITH OR WITHOUT SEIZURES, NEURODEVELOPMENTAL DISORDER, X-LINKED, WITH CRANIOFACIAL ABNORMALITIES, Neuroferritinopathy, Neurofibromatosis, familial spinal, Neurofibromatosis, type 1, Neurofibromatosis, type 2, Neurohypophyseal diabetes insipidus, Neurologic, endocrine, and pancreatic disease, multisystem, infantile-onset, Neuromuscular Diseases, Neuropathy, hereditary motor and sensory, Russe type, NEUROPATHY, HEREDITARY MOTOR AND SENSORY, TYPE VIB, WITH OPTIC ATROPHY, NEUROPATHY, HEREDITARY SENSORY AND AUTONOMIC, TYPE IC, SEVERE, Neuropathy, hereditary sensory and autonomic, type VI, Neuropathy, hereditary sensory and autonomic, type VII, Neuropathy, hereditary sensory and autonomic, type VIII, Neutral 1 amino acid transport defect, Neutral lipid storage disease with myopathy, Neutropenia, severe congenital, 7, autosomal recessive, Nicolaides-Baraitser syndrome, Niemann-Pick disease type C1, Niemann-Pick disease type C2, Niemann-Pick disease, type A, Niemann-Pick disease, type B, Night blindness, congenital stationary, type 1h, Nonepidermolytic palmoplantar keratoderma, Non-Hodgkin lymphoma, Non-immune hydrops fetalis, Sialidosis, type II, Non-ketotic hyperglycinemia, Non-small cell lung cancer, Squamous cell lung carcinoma, Nonsyndromic cleft lip with or without cleft palate, nonsyndromic cleft palate, Nonsyndromic hearing loss and deafness, Rare genetic deafness, Nonsyndromic hypergonadotropic hypogonadism, OVARIAN DYSGENESIS 5, Nonsyndromic Oculocutaneous Albinism, Tyrosinase-negative oculocutaneous albinism, Rasopathy, Tumoral calcinosis, familial, hyperphosphatemic, Type A2 brachydactyly, Type B brachydactyly, Type C brachydactyly, Type IV short rib polydactyly syndrome, Tyrosinase-negative oculocutaneous albinism, Tyrosinase-positive oculocutaneous albinism, Tyrosine kinase 2 deficiency, Tyrosinemia type I, UDPglucose-4-epimerase deficiency, Ullrich congenital muscular dystrophy 1, Uncombable hair syndrome, Unverricht-Lundborg syndrome, Upshaw-Schulman syndrome, Uridine 5-prime monophosphate hydrolase deficiency, hemolytic anemia due to, Urofacial syndrome 2, Uruguay faciocardiomusculoskeletal syndrome, Usher syndrome, Usher syndrome, type 1, Usher syndrome, type 1G, Usher syndrome, type 1J, Usher syndrome, type 1B, Usher syndrome, type 1D, Usher syndrome, type 1F, Usher syndrome, type 2A, Usher syndrome, type 2C, Usher syndrome, type 3A, USHER SYNDROME, TYPE ID/F, DIGENIC, UV-sensitive syndrome 2, UV-sensitive syndrome 3, Van der Woude syndrome, VAN ESCH-O'DRISCOLL SYNDROME, Van Maldergem syndrome 1, Variegate porphyria, Vascular malformation, primary intraosseous, Venous malformation, Ventricular tachycardia, catecholaminergic polymorphic, 2, Ventricular tachycardia, catecholaminergic polymorphic, 5, with or without muscle weakness, Ventricular tachycardia, somatic, Verheij syndrome, Verloes Bourguignon syndrome, Vertical talus, congenital, Very long chain acyl-CoA dehydrogenase deficiency, Vici syndrome, Visceral myopathy, Vitamin D-dependent rickets, type 1, Vitamin D-dependent rickets, type 2, Vitamin k-dependent clotting factors, combined deficiency of, 1, Vitelliform macular dystrophy type 2, Waardenburg syndrome type 1, Waardenburg syndrome type 2A, Waardenburg syndrome type 2E, with neurologic involvement, Waardenburg syndrome type 2E, without neurologic involvement, Waardenburg syndrome type 4A, Waardenburg syndrome type 4B, Waardenburg syndrome type 4C, Wagner syndrome, Walker-Warburg congenital muscular dystrophy, Warburg micro syndrome 1, Warburg micro syndrome 2, Warburg micro syndrome 3, Warburg micro syndrome 4, Warburg-Cinotti syndrome, Warfarin response, Warsaw breakage syndrome, Warts, hypogammaglobulinemia, infections, and myelokathexis, Werdnig-Hoffmann disease, Werner syndrome, WFS1-Related Disorders, White sponge nevus 2, White-sutton syndrome, Wieacker Wolff syndrome, Wiedemann-Steiner syndrome, Wilms tumor 1, Wilson disease, Wiskott-Aldrich syndrome, Witteveen-kolk syndrome, Wolcott-Rallison dysplasia, Wolff-Parkinson-White syndrome, childhood-onset, Wolfram syndrome, Wolman disease, Woolly hair, autosomal dominant, Xerocytosis, Xeroderma pigmentosum and Cockayne syndrome complex, Xeroderma pigmentosum, group C, Xeroderma pigmentosum, group D, Xeroderma pigmentosum, group G, Xeroderma pigmentosum, type 1, Xeroderma pigmentosum, variant type, Xia-Gibbs syndrome, X-linked agammaglobulinemia, X-linked hereditary motor and sensory neuropathy, X-linked hydrocephalus syndrome, X-linked ichthyosis with steryl-sulfatase deficiency, X-linked mental retardation with marfanoid habitus syndrome, X-linked severe combined immunodeficiency, Young Simpson syndrome, Yunis Varon syndrome, Zimmermann-Laband syndrome 1, ZNF711-Related X-linked Mental Retardation, Zonular pulverulent cataract 3, ZTTK syndrome, von Willebrand disease type 2, von Willebrand disease, type 2b, von Willebrand disorder, von Willebrand disease type 1, von Willebrand disease type 3, von Willebrand factor Vicenza, von Willebrand disease type 2N, von Willebrand disease, type 2a, NPHP1-Related Disorders, Nephronophthisis, Nephronophthisis 1, NSDHL-Related Disorders, N-terminal acetyltransferase deficiency, Nuclearly-encoded mitochondrial complex V (ATP synthase) deficiency 1, Nuclearly-encoded mitochondrial complex V (ATP synthase) deficiency 3, Nystagmus 6, congenital, X-linked, Obesity, autosomal dominant, Obesity, mild, early-onset, Schizophrenia, Ochoa syndrome, Ocular albinism, Ocular albinism, type I, Oculoauricular syndrome, Oculocutaneous albinism type 3, OCULOCUTANEOUS ALBINISM, TYPE II, MODIFIER OF, Oculocutaneous albinism type 4, Oculodentodigital dysplasia, Oculodentodigital dysplasia, autosomal recessive, Oculofaciocardiodental syndrome, Oculomaxillofacial dysostosis, Oculomelic amyoplasia, OCULOSKELETODENTAL SYNDROME, O″″DONNELL-LURIA-RODAN SYNDROME, Odontoonychodermal dysplasia, Oguchi disease 2, Oguchi's disease, Retinitis pigmentosa 47, SAG-Related Disorders, Ohdo syndrome, X-linked, Okur-chung neurodevelopmental syndrome, Oligosynaptic infertility, SPERMATOGENIC FAILURE 25, Olivopontocerebellar hypoplasia, Omodysplasia 1, Oocyte maturation defect 1, Oocyte maturation defect 2, OOCYTE MATURATION DEFECT 4, Opitz-Frias syndrome, Opsismodysplasia, Optic atrophy 5, Oral-facial-digital syndrome, Ornithine aminotransferase deficiency, Ornithine carbamoyltransferase deficiency, Orofacial cleft 11, Orofacial cleft 15, Orofacial cleft 6, susceptibility to, Popliteal pterygium syndrome, Van der Woude syndrome, Orofacial clefting, Tooth agenesis, Orofacial-digital syndrome III, Orofaciodigital syndrome 5, Orofaciodigital syndrome xiv, Orofaciodigital syndrome XV, Orotic aciduria, Osler hemorrhagic telangiectasia syndrome, Ossifying fibroma of the jaw, Osteochondritis dissecans, Osteochondrodysplasia, complex lethal, symoens-barnes-gistelinck type, Osteogenesis imperfecta, Osteogenesis imperfecta type 12, Osteogenesis imperfecta type 7, Osteogenesis imperfecta type 8, Osteogenesis imperfecta type 9, Osteogenesis imperfecta type I, Osteogenesis imperfecta type III, Osteogenesis imperfecta with normal sclerae, dominant form, Osteogenesis imperfecta, recessive perinatal lethal, Osteogenesis imperfecta, type VI, Osteogenesis imperfecta, type XI, Osteogenesis imperfecta, type xiii, OSTEOGENESIS IMPERFECTA, TYPE XIX, Osteogenesis imperfecta, type xv, Osteoglophonic dysplasia, Osteomyelitis, sterile multifocal, with periostitis and pustulosis, Osteopathia striata with cranial sclerosis, Osteopetrosis autosomal recessive 1, Osteopetrosis autosomal recessive 2, Osteopetrosis autosomal recessive 4, Osteopetrosis with renal tubular acidosis, Osteopetrosis, autosomal recessive 5, Osteoporosis with pseudoglioma, Otitis media, susceptibility to, Otofaciocervical syndrome 2, Otopalatodigital spectrum disorder, Ovarian dysgenesis 4, Premature ovarian failure 1, Ovarian Neoplasms, Pachyonychia congenita 1, Pachyonychia congenita 2, Steatocystoma multiplex, Pachyonychia congenita 3, Pachyonychia congenita 4, PAGANINI-MIOZZO SYNDROME, Paget disease of bone, familial, Pallister-Hall syndrome, Palmoplantar blistering, Skin fragility with non-scarring blistering, Palmoplantar keratoderma, mutilating, with periorificial keratotic plaques, Palmoplantar keratoderma, nagashima type, Palmoplantar keratoderma, nonepidermolytic, focal 2, Pancreatic agenesis and congenital heart disease, Papillon-Lefevre syndrome, Papulosquamous eruptions, Pityriasis rubra pilaris, Paramyotonia congenita of von Eulenburg, Parathyroid carcinoma, Parietal foramina 1, Parietal foramina 2, Parkinson disease 15, Parkinson disease 19b, early-onset, Parkinson disease 6, autosomal recessive early-onset, Parkinson disease 7, Parkinson disease 8, autosomal dominant, Parkinson disease 9, Parkinson disease, late-onset, Parkinson disease 23, autosomal recessive early-onset, Parkinsonism, early onset with mental retardation, Paroxysmal extreme pain disorder, Paroxysmal nocturnal hemoglobinuria 1, Partial adenosine deaminase deficiency, Severe combined immunodeficiency due to ADA deficiency, Partial albinism, Partial androgen insensitivity syndrome, Patterned dystrophy of retinal pigment epithelium, Peeling skin syndrome, Peeling skin syndrome 5, PEELING SKIN SYNDROME 6, Peeling skin syndrome, acral type, Peeling skin with leukonychia, acral punctate keratoses, cheilitis, and knuckle pads, Pelizaeus-Merzbacher disease, Pendred syndrome, Rare genetic deafness, Peripheral demyelinating neuropathy, central dysmyelination, Waardenburg syndrome, and Hirschsprung disease, PERIPHERAL NEUROPATHY, AUTOSOMAL RECESSIVE, WITH OR WITHOUT IMPAIRED INTELLECTUAL DEVELOPMENT, Periventricular nodular heterotopia 1, Periventricular nodular heterotopia 6, Periventricular nodular heterotopia 7, Periventricular nodular heterotopia with syndactyly, cleft palate and developmental delay, PERIVENTRICULAR NODULAR HETEROTOPIA 8, Permanent neonatal diabetes mellitus, Peroxisome biogenesis disorder 11B, Peroxisome biogenesis disorder 1A (Zellweger), Peroxisome biogenesis disorder 1B, Peroxisome biogenesis disorders, Zellweger syndrome spectrum, Peroxisome biogenesis disorder 3A, Peroxisome biogenesis disorder 4B, Peroxisome biogenesis disorder 4a (zellweger), Peroxisome biogenesis disorder 5a (zellweger), Peroxisome biogenesis disorder 5B, Peroxisome biogenesis disorder 6A, Peroxisome biogenesis disorder 6B, Peroxisome biogenesis disorder, complementation group 7, Peroxisome biogenesis disorder 7A, Peroxisome biogenesis disorder 7B, Peroxisome biogenesis disorder 9B, Rhizomelic chondrodysplasia punctata type 1, Perrault syndrome 3, Perrault syndrome 4, Perrault syndrome 5, Perrault syndrome 6, Perry syndrome, Persistent hyperinsulinemic hypoglycemia of infancy, Persistent hyperplastic primary vitreous, autosomal recessive, Persistent truncus arteriosus, Peters plus syndrome, Pettigrew syndrome, PEX7-Related Disorders, Phytanic acid storage disease, Pfeiffer syndrome, Phenylketonuria, Phosphoglycerate kinase 1 deficiency, Phosphohydroxylysinuria, Phosphoserine aminotransferase deficiency, Pick's disease, Pierson syndrome, Pigmentary disorder, reticulate, with systemic manifestations, X-linked, Pigmentary pallidal degeneration, Pili torti-deafness syndrome, Pineal hyperplasia AND diabetes mellitus syndrome, PINK1-Related Parkinsonism, Pitt-Hopkins syndrome, Severe intellectual deficiency, Pitt-Hopkins-like syndrome 1, PITUITARY ADENOMA 3, MULTIPLE TYPES, PITUITARY ADENOMAS, MULTIPLE TYPES, Pituitary dependent hypercortisolism, Pituitary hormone deficiency, combined 1, Septo-optic dysplasia sequence, Pituitary hormone deficiency, combined 2, Pituitary hormone deficiency, combined 3, Pituitary hormone deficiency, combined 4, Pituitary hormone deficiency, combined 5, Pituitary stalk interruption syndrome, Plasminogen deficiency, type I, Platelet glycoprotein IV deficiency, Platelet-type bleeding disorder 17, Platelet-type bleeding disorder 20, Poikiloderma with neutropenia, Poikiloderma, hereditary fibrosing, with tendon contractures, myopathy, and pulmonary fibrosis, POLG-Related Spectrum Disorders, Progressive sclerosing poliodystrophy, Polyagglutinable erythrocyte syndrome, Polyarteritis nodosa, childhoood-onset, Polycystic kidney disease 2, Polycystic kidney disease 3, Polycystic kidney disease, adult type, Polycystic kidney disease, autosomal dominant, Polycystic lipomembranous osteodysplasia with sclerosing leukoencephalopathy, POLYCYSTIC LIPOMEMBRANOUS OSTEODYSPLASIA WITH SCLEROSING LEUKOENCEPHALOPATHY 2, Polycystic liver disease 1, POLYCYSTIC LIVER DISEASE 3 WITH OR WITHOUT KIDNEY CYSTS, POLYCYSTIC LIVER DISEASE 4 WITH OR WITHOUT KIDNEY CYSTS, Polydactyly, preaxial I, Polydactyly, preaxial II, Triphalangeal thumb, Polyglandular autoimmune syndrome, type 1, Polyglucosan body myopathy 1 with or without immunodeficiency, Polyglucosan body myopathy 2, Polyhydramnios, megalencephaly, and symptomatic epilepsy, POLYMICROGYRIA WITHOUT VASCULAR-TYPE EHLERS-DANLOS SYNDROME, Polymicrogyria, bilateral frontoparietal, Polymicrogyria, bilateral perisylvian, autosomal recessive, Pontocerebellar hypoplasia type 2A, Pontocerebellar hypoplasia type 2B, Pontocerebellar hypoplasia type 6, Pontocerebellar hypoplasia type 8, PONTOCEREBELLAR HYPOPLASIA, TYPE 11, Pontoneocerebellar hypoplasia, Pontocerebellar hypoplasia, type 1 b, Pontocerebellar hypoplasia, type 9, Popliteal pterygium syndrome lethal type, Porencephaly 1, Rhizomelic chondrodysplasia punctata type 1, Rhizomelic chondrodysplasia punctata type 5, RHYNS syndrome, Rigidity and multifocal seizure syndrome, lethal neonatal, Rippling muscle disease 2, Ritscher-schinzel syndrome 2, RLBP1-Related Disorders, Retinitis pigmentosa, Retinitis punctata albescens, Roberts-SC phocomelia syndrome, Robinow syndrome, Robinow syndrome, autosomal dominant 2, Robinow syndrome, autosomal recessive, Robinow syndrome, autosomal recessive, with aplasia/hypoplasia of phalanges and metacarpals/metatarsals, Type B brachydactyly, Robinow syndrome, autosomal dominant 3, Rolandic epilepsy, Rolandic epilepsy with mental retardation and speech dyspraxia, X-linked, Romano-Ward syndrome, RRM2B-related mitochondrial disease, Rubinstein-Taybi syndrome 1, Rubinstein-Taybi syndrome 2, Ruijs-Aalfs syndrome, Russell-Silver syndrome, RYR1-Related Disorders, Saethre-Chotzen syndrome, Salla disease, Sialic acid storage disease, severe infantile type, Salt and pepper developmental regression syndrome, Sandhoff disease, Sandhoff disease, infantile, Sanfilippo syndrome, Scalp ear nipple syndrome, Schaaf-yang syndrome, Schimke immunoosseous dysplasia, Schizophrenia, Schizophrenia 19, Schneckenbecken dysplasia, Schwannomatosis 1, Schwannomatosis 2, SCID due to ADA deficiency, delayed onset, Seckel syndrome 1, Seckel syndrome 10, Seckel syndrome 2, Seckel syndrome 4, Seckel syndrome 5, Seckel syndrome 7, Secondary hypothyroidism, Segawa syndrome, autosomal recessive, Seizures, benign familial infantile, 2, Selective tooth agenesis 1, Sengers syndrome, Senior-Loken syndrome 7, Senior-Loken syndrome 8, Senior-Loken syndrome 9, Sepiapterin reductase deficiency, SeSAME syndrome, SETBP1-Related Disorder, Severe autosomal recessive muscular dystrophy of childhood—North African type, Severe combined immunodeficiency disease, Severe combined immunodeficiency due to ADA deficiency, Severe combined immunodeficiency with sensitivity to ionizing radiation, Severe combined immunodeficiency, athabascan-type, Severe combined immunodeficiency, atypical, Severe combined immunodeficiency, autosomal recessive, T cell-negative, B cell-positive, NK cell-negative, Severe congenital neutropenia, Severe congenital neutropenia 3, autosomal recessive, Severe congenital neutropenia 4, autosomal recessive, Severe congenital neutropenia 5, autosomal recessive, Severe congenital neutropenia X-linked, Severe intellectual deficiency, Severe myoclonic epilepsy in infancy, Severe neonatal-onset encephalopathy with microcephaly, Severe X-linked myotubular myopathy, SHANK3-Related Disorder, Shashi-Pena syndrome, Short QT syndrome 1, Short QT syndrome 2, Short Rib Polydactyly Syndrome, Short-rib thoracic dysplasia 16 with or without polydactyly, Short rib-polydactyly syndrome, Majewski type, Short stature, Short stature with nonspecific skeletal abnormalities, Short stature, auditory canal atresia, mandibular hypoplasia, and skeletal abnormalities, Short stature, idiopathic, autosomal, Short stature, idiopathic, X-linked, Short stature, microcephaly, and endocrine dysfunction, Short stature, rhizomelic, with microcephaly, micrognathia, and developmental delay, SHORT syndrome, Short-rib polydactyly syndrome type I, Short-rib polydactyly syndrome type III, Short-rib thoracic dysplasia 1 with or without polydactyly, Short-rib thoracic dysplasia 15 with polydactyly, Short-rib thoracic dysplasia 10 with or without polydactyly, Short-rib thoracic dysplasia 3 with or without polydactyly, Short-rib thoracic dysplasia 8 with or without polydactyly, Short-rib thoracic dysplasia without polydactyly, Shprintzen syndrome, Shprintzen-Goldberg syndrome, Shwachman syndrome, SHWACHMAN-DIAMOND SYNDROME 2, Sialidosis, type II, Sialuria, Siderius X-linked mental retardation syndrome, Sideroblastic anemia 3, pyridoxine-refractory, Sideroblastic anemia with B-cell immunodeficiency, periodic fevers, and developmental delay, Simpson-Golabi-Behmel syndrome, Wilms tumor 1, Single median maxillary incisor, Singleton-Merten syndrome 2, Sitosterolemia, Sjögren-Larsson syndrome, Skeletal dysplasia, Spondyloepiphyseal dysplasia Maroteaux type, Spondylometaphyseal dysplasia, Kozlowski type, Skin fragility woolly hair syndrome, Skin/hair/eye pigmentation, variation in, 1, SKRABAN-DEARDORFF SYNDROME, Smith-Kingsmore syndrome, Smith-Lemli-Opitz syndrome, Smith-Magenis Syndrome-like, Smith-McCort dysplasia 2, SMS-Related Disorder, Snyder Robinson syndrome, Somatotroph adenoma, Sorsby fundus dystrophy, Sotos syndrome 1, Sotos syndrome 2, Spastic ataxia Charlevoix-Saguenay type, Spastic paraplegia, Spastic paraplegia 1, Spastic paraplegia 11, autosomal recessive, Spastic paraplegia 12, Spastic paraplegia 15, Spastic paraplegia 2, Spastic paraplegia 26, Spastic paraplegia 3, Spastic paraplegia 31, autosomal dominant, Spastic paraplegia 35, Spastic paraplegia 4, autosomal dominant, Spastic paraplegia 42, autosomal dominant, Spastic paraplegia 45, autosomal recessive, Spastic paraplegia 46, autosomal recessive, Spastic paraplegia 47, autosomal recessive, Spastic paraplegia 48, autosomal recessive, Spastic paraplegia 49, autosomal recessive, Spastic paraplegia 5A, Spastic paraplegia 53, autosomal recessive, Spastic paraplegia 54, autosomal recessive, Spastic paraplegia 55, autosomal recessive, Spastic paraplegia 56, autosomal recessive, Spastic paraplegia 6, Spastic paraplegia 63, autosomal recessive, Spastic paraplegia 7, Spastic paraplegia 72, autosomal dominant, Spastic paraplegia 74, autosomal recessive, Spastic paraplegia 75, autosomal recessive, Spastic paraplegia 76, autosomal recessive, Spastic paraplegia 77, autosomal recessive, Spastic paraplegia 79, autosomal recessive, Spastic paraplegia 8, Spastic paraplegia 9, Spastic paraplegia and psychomotor retardation with or without seizures, Spastic paraplegia, intellectual disability, nystagmus, and obesity, Spastic tetraplegia, thin corpus callosum, and progressive microcephaly, Speech-language disorder 1, Spermatogenic failure 11, Spermatogenic failure 16, SPERMATOGENIC FAILURE 18, SPERMATOGENIC FAILURE 20, SPERMATOGENIC FAILURE 22, SPERMATOGENIC FAILURE 23, SPERMATOGENIC FAILURE 25, SPERMATOGENIC FAILURE 27, SPERMATOGENIC FAILURE 31, SPERMATOGENIC FAILURE 33, asthenozoospermia, dysplasia of the mitochondrial sheath, multiple morphologic abnormalities of the sperm flagellum, Spermatogenic failure 9, Spherocytosis type 1, Spherocytosis type 2, Spherocytosis type 4, Spherocytosis type 5, Spherocytosis, type 1, autosomal recessive, Sphingolipid activator protein 1 deficiency, Spiegler-Brooke syndrome, Spinal muscular atrophy with congenital bone fractures 2, Spinal muscular atrophy, distal, autosomal recessive, 1, Spinal muscular atrophy, jokela type, Spinal muscular atrophy, lower extremity-predominant, 2A, autosomal dominant, Spinal muscular atrophy, type II, Werdnig-Hoffmann disease, Spinal muscular atrophy, X-linked 2, Spinocerebellar ataxia 11, Spinocerebellar ataxia 14, Spinocerebellar ataxia 19, Spinocerebellar ataxia 21, Spinocerebellar ataxia 23, Spinocerebellar ataxia 27, Spinocerebellar ataxia 28, Spinocerebellar ataxia 29, Spinocerebellar ataxia 35, Spinocerebellar ataxia 38, SPINOCEREBELLAR ATAXIA 44, Spinocerebellar ataxia autosomal recessive 1, Spinocerebellar ataxia, autosomal recessive 10, Spinocerebellar ataxia, autosomal recessive 14, Spinocerebellar ataxia, autosomal recessive 15, Spinocerebellar ataxia, autosomal recessive 16, Spinocerebellar ataxia, autosomal recessive 17, Spinocerebellar ataxia, autosomal recessive 20, Spinocerebellar ataxia, autosomal recessive 22, Spinocerebellar ataxia, autosomal recessive 8, SPINOCEREBELLAR ATAXIA, AUTOSOMAL RECESSIVE, WITH AXONAL NEUROPATHY 3, Split-hand/foot malformation 1 with sensorineural hearing loss, Split-hand/foot malformation 1, Split-hand/foot malformation 6, Sponastrime dysplasia, Spondylocarpotarsal synostosis syndrome, Spondylocostal dysostosis 1, autosomal recessive, Spondylocostal dysostosis 2, Spondylocostal dysostosis 3, Spondylocostal dysostosis 5, Spondyloenchondrodysplasia with immune dysregulation, SPONDYLOEPIMETAPHYSEAL DYSPLASIA WITH JOINT LAXITY, TYPE 3, Spondyloepimetaphyseal dysplasia with multiple dislocations, Spondyloepimetaphyseal dysplasia, Aggrecan type, Spondyloepimetaphyseal dysplasia, Faden-Alkuraya type, Spondyloepimetaphyseal dysplasia, pakistani type, Spondyloepiphyseal dysplasia, Spondyloepiphyseal dysplasia tarda, Spondyloepiphyseal dysplasia, stanescu type, Spondylo-megaepiphyseal-metaphyseal dysplasia, Spondylometaphyseal dysplasia axial, Spondylometaphyseal dysplasia with cone-rod dystrophy, Spondylometaphyseal dysplasia, megarbane-dagher-melki type, Spondyloocular syndrome, autosomal recessive, Spongy degeneration of central nervous system, sporadic abdominal aortic aneurysm, Stargardt disease 1, Steatocystoma multiplex, Steel syndrome, Stickler syndrome type 1, Stickler syndrome, type 2, Stickler syndrome, type 5, Stiff skin syndrome, Sting-associated vasculopathy, infantile-onset, Stomatocytosis I, Striatal degeneration, autosomal dominant 1, Striatal necrosis, bilateral, and progressive polyneuropathy, Striatonigral degeneration infantile, Stromme syndrome, Structural heart defects and renal anomalies syndrome, Stuve-Wiedemann syndrome, Subcortical laminar heterotopia, X-linked, Succinate-semialdehyde dehydrogenase deficiency, Sucrase-isomaltase deficiency, Sudden cardiac failure, infantile, Sulfite oxidase deficiency, Sulfite oxidase deficiency, isolated, Superoxide dismutase, elevated extracellular, Supravalvar aortic stenosis, Surfactant metabolism dysfunction, pulmonary, 2, Surfactant metabolism dysfunction, pulmonary, 3, SWEENEY-COX SYNDROME, Symphalangism, proximal, 1b, Symphalangism-brachydactyly syndrome, Syndactyly Cenani Lenz type, Syndactyly type 9, Syndromic X-linked mental retardation, Cabezas type, Synpolydactyly 1, Takenouchi-Kosaki syndrome, Tangier disease, TARP syndrome, Tatton-Brown-rahman syndrome, Tay-Sachs disease, Tay-Sachs disease, B1 variant, Temple-Baraitser syndrome, Zimmermann-Laband syndrome 1, Temtamy syndrome, Thiamine metabolism dysfunction syndrome 5 (episodic encephalopathy type), Thoracic aortic aneurysm and aortic dissection, Three M syndrome 1, Thrombocytopenia 5, Thrombocytopenia, X-linked, Thrombocytopenia, X-linked, intermittent, Thrombophilia due to activated protein C resistance, Thrombophilia due to thrombomodulin defect, Thrombophilia, hereditary, due to protein C deficiency, autosomal dominant, Thrombophilia, hereditary, due to protein C deficiency, autosomal recessive, Thrombotic thrombocytopenic purpura, Thyroglobulin synthesis defect, Thyroid dysgenesis, Thyroid dyshormonogenesis 1, Thyroid dyshormonogenesis 6, Thyroid hormone metabolism, abnormal, Thyroid hormone resistance, generalized, Thyroid hormone resistance, generalized, autosomal dominant, Thyroid hormone resistance, generalized, autosomal recessive, Thyroxine-binding globulin, Chicago, Timothy syndrome, TNF receptor-associated periodic fever syndrome (TRAPS), Tooth agenesis, Tooth agenesis, selective, 7, Townes syndrome, Townes-Brocks syndrome 1, Transcobalamin II deficiency, Transcolabamin II deficiency, Transient bullous dermolysis of the newborn, Transient neonatal diabetes mellitus 1, Transient neonatal diabetes mellitus 3, Treacher Collins syndrome 1, Tricho-dento-osseous syndrome, Trichohepatoenteric syndrome 2, Trichomegaly, Trichorhinophalangeal dysplasia type I, Trichorhinophalangeal syndrome type 3, Trichothiodystrophy 1, photosensitive, Trichothiodystrophy 2, photosensitive, Trichothiodystrophy, nonphotosensitive 1, Triglyceride storage disease with ichthyosis, Trimethylaminuria, Triosephosphate isomerase deficiency, Troyer syndrome, Tuberous sclerosis 1, Tuberous sclerosis syndrome, Tubulinopathies, Tumor susceptibility linked to germline BAP1 mutations, Tumoral calcinosis, familial, hyperphosphatemic, Poretti-Boltshauser syndrome, Porokeratosis of Mibelli, Porphyria cutanea tarda, Postaxial polydactyly type A1, Posterior polymorphous corneal dystrophy 1, Potassium aggravated myotonia, Precocious puberty, central, 2, Preimplantation embryonic lethality 2, Premature ovarian failure 10, Premature ovarian failure 3, Premature ovarian failure 5, Premature ovarian failure 8, Primary autosomal recessive microcephaly, Primary autosomal recessive microcephaly 1, Primary autosomal recessive microcephaly 10, Primary autosomal recessive microcephaly 13, Primary autosomal recessive microcephaly 2, Primary autosomal recessive microcephaly 3, Primary autosomal recessive microcephaly 5, Primary ciliary dyskinesia, Primary ciliary dyskinesia 23, Primary ciliary dyskinesia 24, Primary dilated cardiomyopathy, Primary erythromelalgia, Primary familial hypertrophic cardiomyopathy, Primary hyperoxaluria, type I, Primary hyperoxaluria, type II, Primary hyperoxaluria, type III, Primary hypertrophic osteoarthropathy, autosomal recessive 2, Primary hypomagnesemia, Primary localized cutaneous amyloidosis 1, Primary open angle glaucoma juvenile onset 1, Primary progressive aphasia, Primary pulmonary hypertension, Primrose syndrome, Progressive familial intrahepatic cholestasis 2, Progressive familial intrahepatic cholestasis 3, Progressive familial intrahepatic cholestasis 4, Progressive intrahepatic cholestasis, Progressive myoclonus epilepsy with ataxia, Progressive myositis ossificans, Progressive osseous heteroplasia, Pseudohypoparathyroidism, Pseudohypoparathyroidism type 1C, Pseudohypoparathyroidism, type IA, with testotoxicosis, Pseudoneonatal adrenoleukodystrophy, Pseudoprimary hyperaldosteronism, Pseudoxanthoma elasticum, Psoriasis 2, pustular, Psoriasis susceptibility 2, Pulmonary alveolar microlithiasis, Pulmonary arterial hypertension related to hereditary hemorrhagic telangiectasia, Pulmonary fibrosis and/or bone marrow failure, telomere-related, 3, Pulmonary hypertension, primary, dexfenfluramine-associated, Pulmonary veno-occlusive disease, Pyknodysostosis, Pyle metaphyseal dysplasia, Pyogenic arthritis, pyoderma gangrenosum and acne, Pyridoxal 5′-phosphate-dependent epilepsy, Pyridoxine-dependent epilepsy, Pyruvate carboxylase deficiency, Pyruvate dehydrogenase E1-alpha deficiency, Pyruvate dehydrogenase E2 deficiency, Pyruvate dehydrogenase E3-binding protein deficiency, Pyruvate dehydrogenase lipoic acid synthetase deficiency, Pyruvate dehydrogenase phosphatase deficiency, Pyruvate kinase deficiency of red cells, Question mark ears, isolated, Radial aplasia-thrombocytopenia syndrome, Radiohumeral fusions with other skeletal and craniofacial anomalies, Radioulnar synostosis with amegakaryocytic thrombocytopenia, RAG1-Related Disorders, Raine syndrome, Rapadilino syndrome, Rare genetic deafness, Recessive dystrophic epidermolysis bullosa, Recurrent abortion, Reduced antithrombin III activity, Refsum disease, adult, 1, Renal adysplasia, Renal carnitine transport defect, Renal cell carcinoma, papillary, 1, Renal coloboma syndrome, Renal cysts and diabetes syndrome, Renal dysplasia, Renal hamartomas nephroblastomatosis and fetal gigantism, Renal hypodysplasia/aplasia 2, Renal hypodysplasia/aplasia 3, Renal hypouricemia 2, Renal tubular acidosis with progressive nerve deafness, Renal tubular acidosis, distal, autosomal recessive, Renal tubular acidosis, distal, with hemolytic anemia, Renal tubular acidosis, proximal, with ocular abnormalities and mental retardation, Renpenning syndrome 1, Restrictive cardiomyopathy, Reticular dysgenesis, Reticulate acropigmentation of Kitamura, Retinal degeneration, autosomal recessive, clumped pigment type, Retinal dystrophy, Retinal dystrophy with inner retinal dysfunction and ganglion cell abnormalities, Retinal dystrophy, early-onset severe, LRAT-related, Retinal dystrophy, iris coloboma, and comedogenic acne syndrome, Retinitis pigmentosa, Retinitis pigmentosa 10, Retinitis pigmentosa 12, Retinitis pigmentosa 13, Retinitis pigmentosa 14, Retinitis pigmentosa 15, Retinitis pigmentosa 18, Retinitis pigmentosa 19, Retinitis pigmentosa 20, Retinitis Pigmentosa 23, Retinitis pigmentosa 25, Retinitis pigmentosa 26, Retinitis pigmentosa 27, Retinitis pigmentosa 39, Retinitis pigmentosa 4, Retinitis pigmentosa 40, Retinitis pigmentosa 41, Retinitis pigmentosa 46, Retinitis pigmentosa 49, Retinitis pigmentosa 54, Retinitis pigmentosa 56, Retinitis pigmentosa 61, Retinitis pigmentosa 64, Retinitis pigmentosa 68, Retinitis pigmentosa 69, Retinitis pigmentosa 7, Retinitis pigmentosa 71, Retinitis pigmentosa 73, Retinitis pigmentosa 76, Retinitis pigmentosa 77, Retinitis pigmentosa 82 with or without situs inversus, Retinitis pigmentosa and erythrocytic microcytosis, Retinitis pigmentosa, X-linked, and sinorespiratory infections, with deafness, Rett syndrome, Rett syndrome, congenital variant, Rhabdoid tumor predisposition syndrome 1, Rhabdoid tumor predisposition syndrome 2, Rhabdomyosarcoma, RhD category D-VII, Rhd, weak d, type I, Tumoral calcinosis, familial, hyperphosphatemic, Type A2 brachydactyly, Type B brachydactyly, Type C brachydactyly, Type IV short rib polydactyly syndrome, Tyrosinase-negative oculocutaneous albinism, Tyrosinase-positive oculocutaneous albinism, Tyrosine kinase 2 deficiency, Tyrosinemia type I, UDPglucose-4-epimerase deficiency, Ullrich congenital muscular dystrophy 1, Ullrich congenital muscular dystrophy 1, autosomal dominant, Uncombable hair syndrome, Unverricht-Lundborg syndrome, Upshaw-Schulman syndrome, Uridine 5-prime monophosphate hydrolase deficiency, hemolytic anemia due to, Urofacial syndrome 2, Uruguay faciocardiomusculoskeletal syndrome, Usher syndrome, Usher syndrome, type 1, Usher syndrome, type 1G, Usher syndrome, type 1J, Usher syndrome, type 1B, Usher syndrome, type 1D, Usher syndrome, type 1F, Usher syndrome, type 2A, Usher syndrome, type 2C, Usher syndrome, type 3A, UV-sensitive syndrome 2, UV-sensitive syndrome 3, Van der Woude syndrome, Van Maldergem syndrome 1, Variegate porphyria, homozygous, Variegate porphyria, Vascular malformation, primary intraosseous, Venous malformation, Ventricular tachycardia, catecholaminergic polymorphic, 2, Ventricular tachycardia, catecholaminergic polymorphic, 5, with or without muscle weakness, Ventricular tachycardia, somatic, Verheij syndrome, Verloes Bourguignon syndrome, Vertical talus, congenital, Very long chain acyl-CoA dehydrogenase deficiency, Vici syndrome, Visceral myopathy, Vitamin D-dependent rickets, type 1, Vitamin D-dependent rickets, type 2, Vitamin k-dependent clotting factors, combined deficiency of, 1, Vitelliform macular dystrophy type 2, Waardenburg syndrome type 1, Waardenburg syndrome type 2A, Waardenburg syndrome type 2E, with neurologic involvement, Waardenburg syndrome type 2E, without neurologic involvement, Waardenburg syndrome type 4A, Waardenburg syndrome type 4B, Waardenburg syndrome type 4C, Wagner syndrome, Walker-Warburg congenital muscular dystrophy, Warburg micro syndrome 1, Warburg micro syndrome 2, Warburg micro syndrome 3, Warburg micro syndrome 4, Warburg-Cinotti syndrome, Warfarin response, Warsaw breakage syndrome, Warts, hypogammaglobulinemia, infections, and myelokathexis, Werdnig-Hoffmann disease, Werner syndrome, WFS1-Related Disorders, White sponge nevus 2, White-sutton syndrome, Wieacker Wolff syndrome, Wiedemann-Steiner syndrome, Wilms tumor 1, Wilson disease, Wiskott-Aldrich syndrome, Witteveen-kolk syndrome, Wolcott-Rallison dysplasia, Wolff-Parkinson-White syndrome, childhood-onset, Wolfram syndrome, Wolman disease, Woolly hair, autosomal dominant, Xerocytosis, Xeroderma pigmentosum and Cockayne syndrome complex, Xeroderma pigmentosum, group C, Xeroderma pigmentosum, group D, Xeroderma pigmentosum, group G, Xeroderma pigmentosum, type 1, Xeroderma pigmentosum, variant type, Xia-Gibbs syndrome, X-linked agammaglobulinemia, X-linked agammaglobulinemia with growth hormone deficiency, X-linked hereditary motor and sensory neuropathy, X-linked hydrocephalus syndrome, X-linked ichthyosis with steryl-sulfatase deficiency, X-linked mental retardation with marfanoid habitus syndrome, X-linked severe combined immunodeficiency, Young Simpson syndrome, Yunis Varon syndrome, Zimmermann-Laband syndrome 1, ZNF711-Related X-linked Mental Retardation, Zonular pulverulent cataract 3, and ZTTK syndrome.

In some embodiments, the disease associated gene is associated with disorder selected from the group consisting of: Phenylketonuria; Hyperphenylalaninemia; Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; a trinucleotide repeat disorder; a prion disease; Tay-Sachs Disease; heart disease; high blood pressure; Alzheimer's disease; arthritis; diabetes; cancer; and obesity.

In some embodiments, the disease associated gene is associated with sickle cell disease or β-thalassemia, cardiovascular disease, refractory hypertension, malignancies and autoimmune disease, solid tumors and hematological malignancies, acute hepatic porphyria, Type 1 diabetes mellitus refractory T-cell acute lymphoblastic leukemia and T-cell lymphoblastic lymphoma, glycogen storage disease type 1a, Alpha-1 antitrypsin deficiency, p47\hat{ }phox Chronic Granulomatous Disease, X-linked Chronic Granulomatous Disease, Wilson's Disease, and cystic fibrosis.

### Target Nucleic Acid

As used herein, the term “target nucleic acid” refers to any nucleic acid whose sequence is be edited or changed. For example, the target nucleic acid can be a cellular gene whose expression is associated with a particular disorder or disease state. Generally, the target nucleic acid is double-stranded target nucleic acid e.g., double-stranded DNA. The double-stranded target nucleic acid, e.g., DNA comprises a “non-target strand” and a “target strand.” The target-strand is the strand that becomes annealed to the spacer of a pegRNA. Optionally, the non-target strand may comprise a protospacer adjacent motif (PAM), which is a short nucleotide sequence (e.g., DNA sequence, such as NGG) located downstream of the target sequence that may be needed for a napDNAbp to bind and cleave target nucleic acid, e.g., cleave the non-target strand. The target strand is also referred to as the “non-edit strand” or the “non-PAM strand herein. The non-target strand is also referred to as the “edit strand” or the “PAM-strand” herein.

In some embodiments of any of the aspects, the target nucleic acid is DNA. For example, the target nucleic acid is double-stranded DNA. In some preferred embodiments, the target nucleic acid is genomic DNA, episomal DNA, viral DNA, or bacterial DNA.

In some embodiments of any of the aspects, the target nucleic acid is RNA. For example, the target nucleic acid is double-stranded RNA. In some embodiments, the target nucleic acid is RNA selected from the group consisting of messenger RNA (mRNA), pre-mRNA, ribosomal RNA (rRNA), transfer RNA (tRNA), micro-RNA (miRNA), small nuclear RNA (snRNA), small nucleolar RNA (snoRNA), non-coding RNA (ncRNA), long non-coding RNA (lncRNA), and small cytoplasmatic RNA (scRNA).

In some embodiments, the target nucleic acid is a gene selected from the group consisting of: ACADVL, ACTG2, ADAMTS13, ADGRV1, AHDC1, ATP7B, BEST1, BMPR1B, BTK, CASQ2, CDH23, CIB2, CLRN1, COL6A1, CSTB, CXCR4, CYP27B1, CYP2C9, DCHS1, DDR2, DDX11, EDN3, EDNRB, EIF2AK3, ELMO2, EPG5, ERCC2, ERCC5, ERCC8, FAH, FHL1, FIG4, FKRP, FKTN, GALE, GALNT3, GDF5, GGCX, GJA3, GJB1, GLMN, GNAI2, GPC3, HOXD10, IFT80, IGHMBP2, IL2RG, IRF6, KAT6B, KCNH1, KMT2A, KRT13, KRT74, L1CAM, LIPA, LRIG2, LTBP3, MED12, MITF, MYLK, MYO7A, NT5C3A, OCA2, PAD13, PAX3, PCDH15, PIEZO1, POGZ, POLA1, POLH, PPOX, PRKAG2, PUF60, RAB18, RAB3GAP1, RAB3GAP2, ROR2, SIN3A, SMN1, SON, SOX10, STS, TBC1D20, TRDN, TYK2, TYR, USH1G, USH2A, UVSSA, VCAN, VDR, WAS, WDR19, WFS1, WRN, WT1, XPA, XPC, ZC4H2, ZNF711, ABCA4, ABCB7, ABCD1, ADSL, AGTPBP1, AK1, AKR1C2, ALG12, ALMS1, ALPL, ALS2, AMACR, AMELX, ANG, ANXA11, APOA1, APP, APRT, AR, ARID1B, ATF6, ATP1A3, AUH, BCL11A, BICD2, BRAF, CAPN3, CASK, CBX2, CCDC65, CDK10, CDKL5, CEBPA, CFI, CHMP2B, CLN6, CLPB, CNGA3, CNGB3, COL17A1, COL2A1, COL4A1, COL4A3, COL4A4, COL4A5, COL7A1, CPAMD8, CRYAB, CTNNB1, DCTN1, DCX, DDX41, DHCR7, DHH, DHTKD1, DLL4, DLX3, DNAJC19, DOCK6, DYRK1A, DYRK1B, EOGT, ETV6, F9, FAM83H, FBLN5, FBN1, FGA, FGB, FGFR2, FGG, G6PD, GATA1, GATA6, GBA, GBE1, GFAP, GMPPA, GPNMB, GPR68, HBA2, HBB, HDAC8, HGD, HMBS, HMCN1, HPD, HSD17B10, HSD3B2, HSPA9, HTRA2, IFIH1, IGFALS, IMPG2, ITGB6, JAG1, KCNJ2, KCNK4, KIAA1109, KIF7, KLK4, LAMB3, MCCC1, MCCC2, MYF5, MYH11, NAGLU, NOTCH1, NOTCH2, NPHP3, NPR2, NROB1, NR5A1, NSD1, ODAPH, OPA1, OPA3, OTC, PAX6, PDE4D, PDE6C, PRR12, PSEN1, PTPN11, PTS, PXDN, RHOA, RMRP, RNASEH2A, RNASEH2B, RNASEH2C, SAMHD1, SBDS, SEPTIN9, SERAC1, SERPINA1, SERPINF2, SETX, SF3B1, SHANK3, SIGMAR1, SLC12A6, SLC16A2, SLC25A38, SLC26A2, SLURP1, SMARCAD1, SMCHD1, SMG9, SOD1, SOS1, SPG11, SPTB, SRD5A2, SRY, SYTL2, SZT2, TANGO2, TARDBP, TAZ, TBX19, TBX22, TGM6, TP63, TREX1, UBE3A, UBQLN1, VCP, VPS13B, VWF, WDR45, WDR7, ABCA12, ABCB11, ACOX2, ACTB, ACTG1, ACVR1B, ADGRG6, AGA, AIRE, ALOX12B, ALOXE3, ANKRD11, APOC2, APOE, APTX, ARG1, ARL6, ARMC9, ARSA, ASL, ASNS, ASPA, ATP2A1, ATRX, BAAT, BAP1, BAX, BBIP1, BBS1, BBS10, BBS12, BBS2, BBS4, BBS5, BBS7, BBS9, BICD2, BIN1, BLM, BSND, BTD, C1QB, C1QC, C8orf37, CA5A, CACNA1C, CACNA1G, CARD9, CD46, CDKN1C, CEP290, CFH, CFTR, CHD8, CHRNA4, CHRND, CLCN1, CLCNKB, CLPP, COL1A1, COL6A2, COL6A3, COLGALT1, COPA, CPT2, CRH, CRIPT, CRX, CTNNA3, CTNND1, CYBB, CYP4V2, DGKE, DMD, DNAJC21, DRD4, DSC2, EDAR, EDARADD, EFEMP2, ENPP1, EP300, EPHB4, ERGIC1, FBLN5, FLNB, FOXC1, FOXL2, FOXP1, GATA3, GATM, GDF1, GH1, GJA5, GJB2, GNAI3, GP1BA, GP9, H3-3A, H3C11, HINT1, HIVEP1, HNRNPK, HOXD13, HSD11B2, HSD17B4, HSD3B7, IFT27, ITCH, KCNH2, KCNJ1, KCNK9, KCNQ1, KCNQ2, KCNQ3, KLF1, KRT1, KRT10, LMNA, LOC110806306; TERC, LRP1, LZTFL1, MAGED2, MC4R, MECP2, MED25, MEF2C, MKKS, MKS1, MYBPC3, MYH3, MYH6, MYH9, NDP, NF1, NIPAL4, NKX2-1, NKX2-5, NOD2, NPPA, PHF6, PIEZO2, PITX2, PKD1, PKP2, PLS3, PMM2, PNKP, PNPLA1, PNPLA6, POLG, PPARG, PPP3CA, PRKAR1A, PROM1, PRPS1, PRX, RASA1, RECQL4, RFXAP, RGS9, RHO, RLBP1, RNF135, SCN5A, SCYL1, SDCCAG8, SGMS2, SIX1, SLC12A1, SLC19A3, SLC26A4, SLC2A10, SLC34A3, SLC35A3, SLC52A2, SLC52A3, SMAD4, SMARCAD1, SMARCD2, SMCHD1, TBC1D24, TBX5, TERC, TERT, TFAP2A, TGM1, THOC6, TLL1, TMEM43, TMEM67, TNFAIP3, TRPV4, TSC1, TSPAN12, TTC21B, TTC8, TTPA, TUBB4A, TWNK, TXNL4A, UFSP2, VAMP1, VIPAS39, AARS1, AARS2, ABCA4, ABCB4, ABL1, ACSF3, ACTG2, AGK, AHDC1, AIFM1, APP, ARID1B, ARID2, ARSL, ASS1, ASXL1, ATP1A1, ATP8B1, BAG3, BCAP31, BRAF, BRF1, BSCL2, C12orf65, C1QBP, C7, C8B, C8orf37, C9, CACNA2D2, CARMIL2, CARS2, CASQ2, CATSPER1, CBS, CC2D2A, CCDCl03, CCDCl14, CCDCl51, CCDC40, CCM2, CCNO, CD19, CERKL, CFAP298, CFHR5, CHD7, CHM, CHRNG, CLCF1, CLCN4, CLN3, CLN5, CLN6, CLN8, COG7, COL1A2, COL3A1, COQ2, COQ4, COQ6, COQ7, COQ8A, COQ9, CP, CPT1A, CPT2, CR2, CRLF1, CRX, CRYAA, CRYBA40, CRYBB1, CRYBB2, CRYBB3, CRYGB, CRYGC, CRYGD, CTC1, CTDP1, CTSA, CTSD, CTSF, CYP17A1, CYP21A2, CYP27A1, DHTKD1, DNAAF1, DNAAF2, DNAAF3, DNAAF5, DNAH1, DNAH5, DNA12, DNAJB13, DNAJC5, DNAL1, DNM2, DNMT3B, DPF2, DRC1, DSP, DYNC1H1, EBP, EED, EGR2, EHMT1, ELAC2, ELP1, EMC1, ERCC2, ERCC5, ERCC8, EVC, EVC2, FARS2, FARSB, FBN1, FGD4, FGFR1, FIG4, FLNA, FLNC, FOLR1, FRRS1L, GAD1, GAMT, GARS1, GATB, GDAP1, GFM1, GFM2, GJA8, GJB1, GLA, GNB1, GRN, GTPBP3, GUCY2D, HARS1, HDAC6, HNF1B, HNRNPA1, HSPB1, HSPB8, HTRA1, HYDIN, IGHMBP2, IL2RG, IMPAD1, INF2, KCNH2, KCNQ1, KDM1A, KIF1B, KLHL7, KRIT1, KRT8, LEMD2, LITAF, LMNA, LRRC6, LRSAM1, LSS, LYST, MAB21L1, MAF, MAP2K1, MAP2K2, MAP3K7, MARS1, MECR, MEGF8, MFN2, MPV17, MPZ, MRPL44, MTFMT, MTHFD1, MTMR2, MTO1, MYBPC3, MYH7, MYO5A, NAGLU, NARS2, NCF1, NCF2, NECTIN1, NEFH, NEFL, NFKB2, NKX2-1, NLRP3, NME8, NOTCH3, NSDHL, NTRK1, OPN1LW, PBRM1, PDCD10, PDSS1, PDSS2, PGAP1, PKP2, PLD1, PLEKHG5, PLOD1, PMP2, PMP22, PNPT1, PPCS, PPT1, PRKAG2, PRKAR1A, PRNP, PROM1, PRPH2, PRPS1, PSAP, PTPN11, RAB23, RAB28, RAB7A, RAF1, RAG1, RAG2, RAX2, RBM20, RETREG1, RPGRIP1, RPGRIP1L, RPS6KA3, RSPH3, RSPH4A, RUBCN, RUNX2, RYR2, SATB2, SBF1, SCN5A, SCN8A, SEC24D, SEMA4A, SETX, SEZ6, SFXN4, SH3TC2, SIX6, SLC25A1, SLC25A13, SLC25A20, SLC25A26, SLC2A1, SLC9A6, SMAD3, SMARCE1, SNAP29, SNRPB, SOX4, SPAG1, SPG11, SPG7, SPINK1, SPTLC1, STAR, STN1, STXBP1, TAZ, TBC1D24, TBX22, TCF4, TDRD7, TFRC, TGDS, TGFB2, TGFBR1, TMEM67, TNFRSF13B, TNN13, TNN13K, TNNT2, TPP1, TRDN, TTLL5, TTN, TUFM, UQCRFS1, VARS2, VLDLR, VPS13A, VPS13B, ZMYND10, ABCA4, ACADS, ACADSB, ACAT1, ACTA1, ADGRG2, AGPAT2, AGRN, AIFM1, AIPL1, AKT1, ALDH18A1, ALDH4A1, ALG1, ALG11, ALG2, ALG3, ALG6, ALG8, ANKH, APOC3, ATN1, ATP2A2, ATP6VOA2, ATP7A, BCHE, BCOR, BPGM, BRAF, BSCL2, CACNA1C, CACNA1F, CCDCl15, CCDC88C, CDAN1, CDH23, CDK13, CEACAM16, CELA2A, CEP250, CEP78, CERKL, CFTR, CHAT, CHRNA1, CHRNE, CLCN1, CLDN14, CLIC5, CNNM4, CNTNAP1, COCH, COG2, COG4, COL11A2, COL8A2, COLQ, CPS1, CRB1, CRLF1, CRPPA, CRX, CRYAA, CRYGD, CRYM, CTH, CTNNB1, CTNS, CYLD, CYP11B1, CYP17A1, D2HGDH, DCC, DCDC2, DCN, DDC, DHDDS, DIAPH1, DOLK, DPAGT1, DPM1, DPM3, EFNB1, EGR2, ELANE, EPS8, EPS8L2, ERF, FAM161A, FBN2, FCSK, FGF10, FGF3, FKRP, FKTN, FRAS1, FREM2, FRMD4A, GALK1, GALT, GATA4, GCNT2, GFPT1, GIPC3, GJA3, GJA8, GJB2, GJB3, GJB6, GI12, GNAQ, GNAT1, GPR179, GRAP, GRHL2, GRM6, GRXCR1, GRXCR2, GSDME, GUCY2D, H6PD, HAAO, HDAC8, HMGCL, IFT122, KARS1, KCNE2, KCNH2, KCNJ2, KCNQ1, KERA, KIF2A, KIF5C, KIT, KITLG, LAMA2, LAMP2, LARGE1, LCT, LOXHD1, LTBP4, MAN2B1, MAP2K1, MARVELD2, MET, MIP, MNX1, MPL, MSRB3, MSX2, MYH3, MYH9, MYMK, MYO15A, MYO3A, MYO5B, MYO6, MYO7A, NALCN, NGLY1, NHS, NIPBL, NKX2-5, NLRP3, NR0B1, NR2F2, NRAS, NYX, OSBPL2, OTOA, OTOF, OTOG, PAX6, PCDH15, PDE6B, PEX1, PGM1, PHOX2B, POC1B, POMGNT1, POMT1, POU3F4, POU4F3, PRKD1, PSMC3, PTPRQ, RAD21, RAPSN, RFT1, RHO, RPGR, RPIA, RYR1, S1PR2, SCN4A, SCN5A, SEC23B, SLC24A1, SLC26A3, SLC26A4, SLC26A5, SLC33A1, SLC35A2, SLC35C1, SLC39A8, SLC3A1, SLC46A1, SLC4A11, SLC5A1, SLC6A8, SLC7A9, SLITRK6, SMC1A, SMC3, SMPX, SSR4, STRC, SYNE4, TAB2, TALDO1, TBC1, and variants and mutated forms thereof.

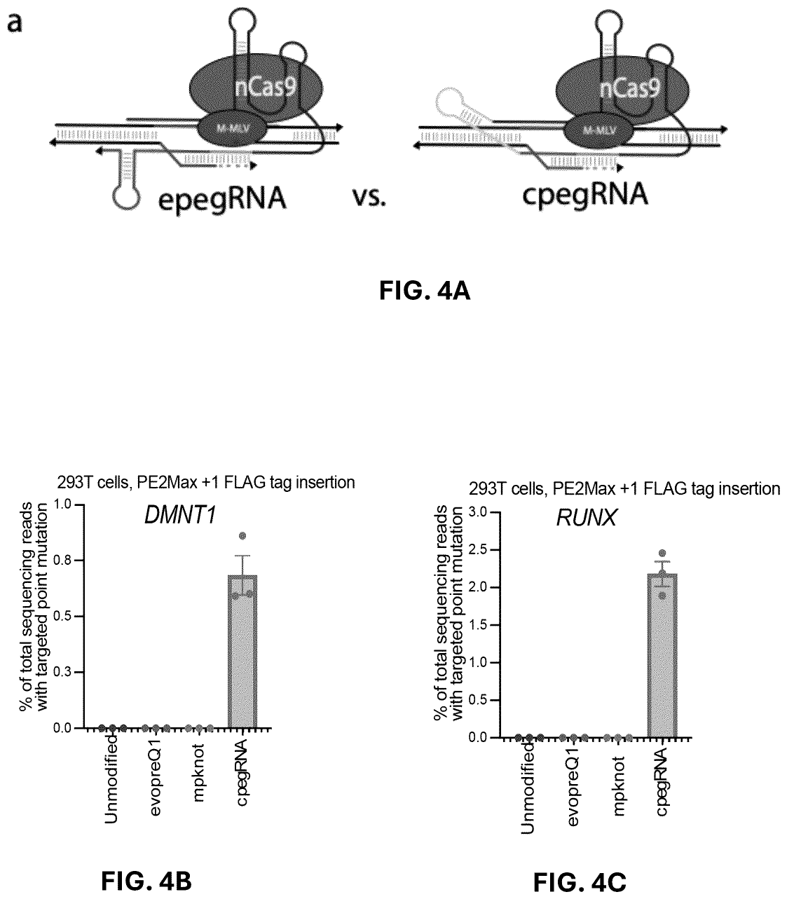

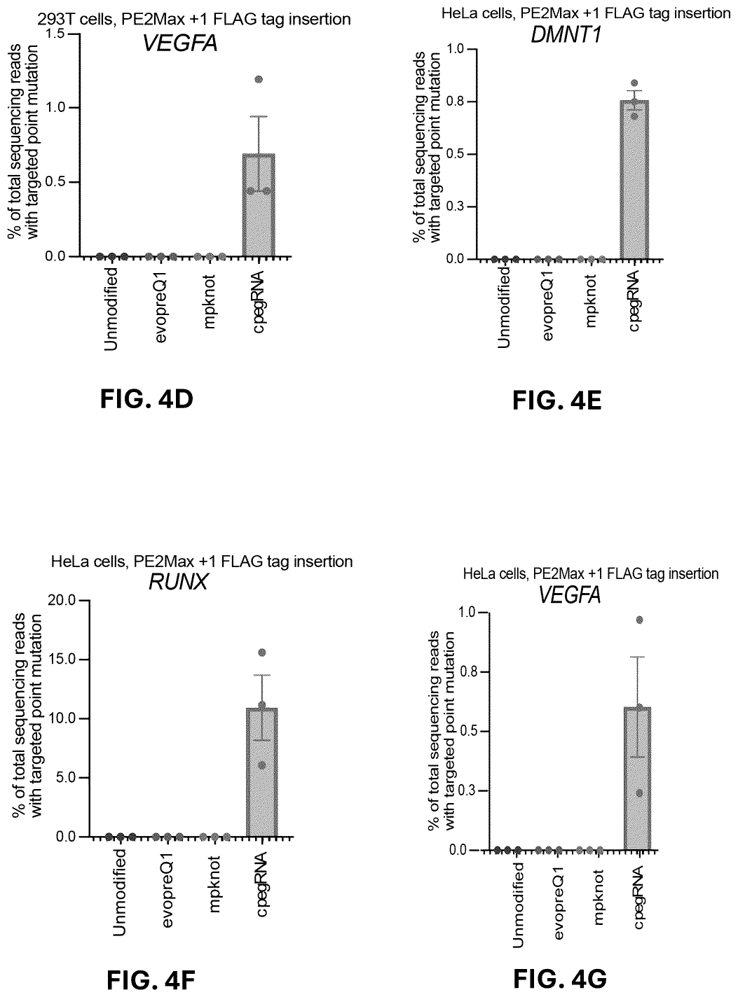

In some embodiments, the target nucleic acid is a mutated gene selected from the group consisting of: NM_000018.4(ACADVL):c.1372T>C (p.Phe458Leu), NM_000018.4(ACADVL):c.864del (p.Phe288fs), NM_000018.4(ACADVL):c.1144A>C (p.Lys382Gln), NM_000018.4(ACADVL):c.799_802del (p.Val267fs), NM_000018.4(ACADVL):c.708_709del (p.Cys237fs), NM_000018.4(ACADVL):c.644_647del (p.Phe214_Cys215insTer), NM_000018.4(ACADVL):c.497_498del (p.IIe166fs), NM_000018.4(ACADVL):c.1141_1143del (p.Glu381del), NM_000018.4(ACADVL):c.996del (p.Ala333fs), NM_000018.4(ACADVL):c.848T>C (p.Val283Ala), NM_000018.4(ACADVL):c.1322G>A (p.Gly441Asp), NM_000018.4(ACADVL):c.1843C>T (p.Arg615Ter), NM_000018.4(ACADVL):c.753-2A>C, NM_000018.3(ACADVL):c.1375dup, NM_000018.4(ACADVL):c.887_888del (p.Pro296fs), NM_001615.4(ACTG2):c.187C>G (p.Arg63Gly), NM_001615.4(ACTG2):c.255+210C>A, NM_001615.4(ACTG2):c.400T>A (p.Tyr134Asn), NM_001615.4(ACTG2):c.134T>C (p.Met45Thr), NM_139025.4(ADAMTS13):c.2851T>G (p.Cys951Gly), NM_139025.4(ADAMTS13):c.3070T>G (p.Cys1024Gly), NM_139025.4(ADAMTS13):c.3770dup (p.Leu1258fs), NM_139025.4(ADAMTS13):c.4143dup (p.Glu1382fs), NM_139025.4(ADAMTS13):c.1783_1784del (p.Leu595fs), NM_139025.4(ADAMTS13):c.2931_2936del (p.Cys977_Arg979delinsTrp), NM_139025.4(ADAMTS13):c.2017A>T (p.IIe673Fhe), NM_032119.4(ADGRV1):c.17200G>T (p.Glu5734Ter), NM_032119.4(ADGRV1):c.8790del (p.Met2931fs), NM_032119.4(ADGRV1):c.2258_2270del (p.Gln753fs), NM_001029882.3(AHDC1):c.1519A>T (p.Lys507Ter), NM_001029882.3(AHDC1):c.3497C>G (p.Ser1166Ter), NM_000053.4(ATP7B):c.3796G>A (p.Gly1266Arg), NM_000053.4(ATP7B):c.1847G>A (p.Arg616Gln), NM_000053.4(ATP7B):c.845del (p.Leu282fs), NM_000053.4(ATP7B):c.3904-2A>G, NM_000053.4(ATP7B):c.3305T>C (p.IIe1102Thr), NM_000053.4(ATP7B):c.994G>T (p.Glu332Ter), NM_000053.4(ATP7B):c.2297C>G (p.Thr766Arg), NM_000053.4(ATP7B):c.383del (p.Gly128fs), NM_000053.4(ATP7B):c.2304del (p.Met769fs), NM_000053.3(ATP7B):c.−441_-427del15, NM_000053.4(ATP7B):c.51+4A>T, NM_000053.4(ATP7B):c.1782del (p.Thr593_Tyr594insTer), NM_000053.4(ATP7B):c.3942_3943del (p.Lys1315fs), NM_000053.4(ATP7B):c.1374_1377del (p.Val459fs), NM_000053.4(ATP7B):c.1340_1343del (p.Gln447fs), NM_000053.4(ATP7B):c.3800del (p.Asp1267fs), NM_000053.4(ATP7B):c.3207C>A (p.His1069Gln), NM_000053.4(ATP7B):c.2333G>T (p.Arg778Leu), NM_000053.4(ATP7B):c.3809A>G (p.Asn1270Ser), NM_000053.4(ATP7B):c.2128G>A (p.Gly710Ser), NM_000053.4(ATP7B):c.3402del (p.Ala1135fs), NM_000053.4(ATP7B):c.2930C>T (p.Thr977Met), NM_000053.4(ATP7B):c.2305A>G (p.Met769Val), NM_000053.4(ATP7B):c.2975C>T (p.Pro992Leu), NM_000053.4(ATP7B):c.2532del (p.Val845fs), NM_000053.4(ATP7B):c.2906G>A (p.Arg969Gln), NM_000053.4(ATP7B):c.2810del (p.Val937fs), NM_000053.4(ATP7B):c.1745_1746del (p.IIe582fs), NM_000053.4(ATP7B):c.2336G>A (p.Trp779Ter), NM_000053.4(ATP7B):c.2123T>C (p.Leu708Pro), NM_004183.4(BEST1):c.172_173dup (p.Gln58fs), NM_004183.4(BEST1):c.728C>T (p.Ala243Val), NM_004183.4(BEST1):c.16A>C (p.Thr6Pro), NM_004183.4(BEST1):c.679T>A (p.Tyr227Asn), NM_001203.3(BMPR1B):c.599T>A (p.IIe200Lys), NM_000061.2(BTK):c.310-1G>C, NM_000061.2(BTK):c.338T>A (p.Val113Asp), NM_000061.2(BTK):c.310-2A>G, NM_000061.2(BTK):c.97A>C (p.Thr33Pro), NM_000061.2(BTK):c.389del (p.Asn130fs), NM_000061.2(BTK):c.228_231del (p.Glu76fs), NM_000061.2(BTK):c.141+3_141+4del, NM_000061.2(BTK):c.1004T>A (p.Val335Asp), NM_000061.2(BTK):c.655del (p.Val219fs), NM_000061.2(BTK):c.799_806del (p.Asn267fs), NM_000061.2(BTK):c.1673_1680del (p.Lys558fs), NM_000061.2(BTK):c.1526T>C (p.Met509Thr), NM_000061.2(BTK):c.1125T>G (p.Tyr375Ter), NM_000061.2(BTK):c.1685G>C (p.Arg562Pro), NM_000061.2(BTK):c.557dup (p.Pro187fs), NM_000061.2(BTK):c.472_475del (p.Thr158fs), NM_000061.2(BTK):c.215dup (p.Asn72fs), NM_001232.3(CASQ2):c.919G>C (p.Asp307His), NM_001232.3(CASQ2):c.62del (p.Glu21fs), NM_022124.6(CDH23):c.6000C>A (p.Tyr2000Ter), NM_022124.6(CDH23):c.8239del (p.Val2747fs), NM_022124.6(CDH23):c.193del (p.Leu65fs), NM_006383.4(CIB2):c.192G>C (p.Glu64Asp), NM_052995.2(CLRN1):c.231_233del (p.IIe77_Leu78delinsMet), NM_001848.2(COL6A1):c.1977C>G (p.Tyr659Ter), NM_001848.2(COL6A1):c.857del (p.Pro286fs), NM_001848.2(COL6A1):c.1465del (p.Ala489fs), NM_001848.2(COL6A1):c.868G>C (p.Gly290Arg), NM_000100.3(CSTB):c.10G>C (p.Gly4Arg), NM_000100.3(CSTB):c.212A>C (p.Gln71Pro), NM_001008540.2(CXCR4):c.1028_1029del (p.Ser343fs), NM_000785.4(CYP27B1):c.1165C>G (p.Arg389Gly), NM_000785.4(CYP27B1):c.962C>G (p.Thr321Arg), NM_000785.4(CYP27B1):c.1004G>C (p.Arg335Pro), NM_000785.4(CYP27B1):c.631del (p.Glu211fs), NM_000785.4(CYP27B1):c.262del (p.Val88fs), NM_000771.4(CYP2C9):c.622T>G (p.Leu208Val), NM_003737.4(DCHS1):c.2503G>T (p.Gly835Ter), NM_003737.4(DCHS1):c.2543del (p.Thr848fs), NM_003737.4(DCHS1):c.7109A>T (p.Asn2370IIe), NM_006182.4(DDR2):c.2219A>G (p.Tyr740Cys), NM_006182.4(DDR2):c.1829T>C (p.Leu610Pro), NM_030653.4(DDX11):c.2576T>G (p.Val859Gly), NM_030653.4(DDX11):c.2689_2691del (p.Lys897del), NM_207034.3(EDN3):c.277C>G (p.Arg93Gly), NM_207034.3(EDN3):c.507C>A (p.Cys169Ter), NM_207034.3(EDN3):c.335A>G (p.His112Arg), NM_001122659.3(EDNRB):c.548C>G (p.Ala183Gly), NM_004836.7(EIF2AK3):c.1035dup (p.Lys346Ter), NM_004836.7(EIF2AK3):c.1564_1565del (p.Trp522fs), NM_133171.5(ELMO2):c.2080del (p.Leu694fs), NM_020964.3(EPG5):c.6232C>T (p.Arg2078Ter), NM_020964.3(EPG5):c.2575G>T (p.Glu859Ter), NM_020964.3(EPG5):c.5938G>T (p.Glu1980Ter), NM_020964.3(EPG5):c.4665del (p.Glu1555fs), NM_000400.3(ERCC2):c.184-1G>T, NM_000400.3(ERCC2):c.1621A>C (p.Ser541Arg), NM_000123.3(ERCC5):c.348_352del (p.Arg116fs), NM_000123.3(ERCC5):c.1115_1118del (p.Arg372fs), NM_000123.3(ERCC5):c.1494del (p.Asp499fs), NM_000123.3(ERCC5):c.2751del (p.Lys917fs), NM_000123.3(ERCC5):c.2878G>T (p.Glu960Ter), NM_000082.3(ERCC8):c.1083G>T (p.Trp361Cys), NM_000137.3(FAH):c.786G>A (p.Trp262Ter), NM_000137.3(FAH):c.47A>T (p.Asn16IIe), NM_000137.3(FAH):c.698A>T (p.Asp233Val), NM_000137.3(FAH):c.615del (p.Phe205fs), NM_000137.3(FAH):c.438del (p.Asn146fs), NM_000137.3(FAH):c.1190del (p.Gn397fs), NM_000137.3(FAH):c.1062+5G>A, NM_000137.3(FAH):c.554-1G>T, NM_000137.3(FAH):c.192G>T (p.Gn64His), NM_001159699.2(FHL1):c.550-2A>G, NM_014845.5(FIG. 4):c.1260_1261del (p.Thr422fs), NM_014845.5(FIG. 4):c.831_838del (p.Lys278fs), NM_024301.5(FKRP):c.1083C>A (p.Tyr361Ter), NM_006731.2(FKTN):c.1153A>T (p.Lys385Ter), NM_001008216.2(GALE):c.548T>C (p.Leu183Pro), NM_001008216.2(GALE):c.101A>G (p.Asn34Ser), NM_004482.4(GALNT3):c.803dup (p.Thr269fs), NM_004482.4(GALNT3):c.677del (p.Ala226fs), NM_000557.5(GDF5):c.1461T>G (p.Tyr487Ter), NM_000821.7(GGCX):c.215-1G>T, NM_000821.7(GGCX):c.1181T>G (p.Leu394Arg), NM_021954.4(GJA3):c.563A>T (p.Asn188IIe), NM_021954.4(GJA3):c.563A>C (p.Asn188Thr), NM_000166.6(GJB1):c.259C>G (p.Pro87Ala), NM_053274.3(GLMN):c.108C>A (p.Cys36Ter), NM_002070.4(GNAI2):c.600T>A (p.Phe200Leu), NM_004484.3(GPC3):c.1471G>T (p.Glu491Ter), NM_002148.3(HOXD10):c.956T>A (p.Met319Lys), NM_020800.3(IFT80):c.487_490del (p.Leu163fs), NM_002180.2(IGHMBP2):c.707T>G (p.Leu236Ter), NM 002180.2(IGHMBP2):c.675del (p.Glu226fs), NM_000206.2(IL2RG):c.186T>A (p.Cys62Ter), NM_000206.2(IL2RG):c.458T>A (p.IIe153Asn), NM_000206.2(IL2RG):c.903_910del (p.Glu302fs), NM_000206.2(IL2RG):c.703_711dup (p.Gln235_Trp237dup), NM_000206.2(IL2RG):c.87del (p.Asn31fs), NM_000206.2(IL2RG):c.865C>T (p.Arg289Ter), NM_000206.2(IL2RG):c.677G>A (p.Arg226His), NM_000206.2(IL2RG):c.270-15A>G, NM_006147.4(IRF6):c.274G>T (p.Glu92Ter), NM_006147.4(IRF6):c.1195del (p.Ala399fs), NM_012330.4(KAT6B):c.3216del (p.Glu1073fs), NM_012330.4(KAT6B):c.4584del (p.Glu1529fs), NM_012330.4(KAT6B):c.3962_3963del (p.Gln1321fs), NM_012330.4(KAT6B):c.4405dup (p.Ser1469fs), NM_012330.4(KAT6B):c.3018del (p.Glu1007fs), NM_000350.3(ABCA4):c.4139C>T (p.Pro1380Leu), NM_000350.3(ABCA4):c.5714+5G>A, NM_000350.3(ABCA4):c.4469G>A (p.Cys1490Tyr), NM_000350.3(ABCA4):c.32T>C (p.Leu11Pro), NM_000350.3(ABCA4):c.5461-10T>C, NM_000350.3(ABCA4):c.6088C>T (p.Arg2030Ter), NM_000350.3(ABCA4):c.6118C>T (p.Arg204OTer), NM_000350.3(ABCA4):c.5917del (p.Gly1972_Val1973insTer), NM_004299.6(ABCB7):c.1234G>C (p.Val412Leu), NM_001271696.3(ABCB7):c.1200T>G (p.IIe400Met), NM_000033.4(ABCD1):c.1850G>A (p.Arg617His), NM_000033.4(ABCD1):c.1628C>T (p.Pro543Leu), NM 000033.4(ABCD1):c.1772G>A (p.Arg591Gln), NM_005157.6(ABL1):c.677A>G (p.Tyr226Cys), NM_014049.5(ACAD9):c.130T>A (p.Phe44IIe), NM_014049.5(ACAD9):c.976G>C (p.Ala326Pro), NM_014049.5(ACAD9):c.359del (p.Phe120fs), NM_001613.4(ACTA2):c.536G>A (p.Arg179His), NM_001613.4(ACTA2):c.353G>A (p.Arg118Gln), NM_001613.4(ACTA2):c.773G>A (p.Arg258His), NM_000666.3(ACY1):c.699A>C (p.Glu233Asp), NM_000666.3(ACY1):c.1001_1001+5del, NM_001111.5(ADAR):c.3335A>T (p.Tyr1112Phe), NM_000026.4(ADSL):c.1312T>C (p.Ser438Pro), NM_000026.4(ADSL):c.674T>C (p.Met225Thr), NM_000026.4(ADSL):c.1277G>A (p.Arg426His), NM_000026.4(ADSL):c.340T>C (p.Tyr114His), NM_000026.4(ADSL):c.568C>T (p.Arg190Ter), NM_000026.4(ADSL):c.1339T>C (p.Ser447Pro), NM_001286715.1(AGTPBP1):c.2492-1G>T, NM_000476.2(AK1):c.139del (p.Val47fs), NM_001354.5(AKR1C2):c.666T>G (p.His222Gln), NM_001354.5(AKR1C2):c.270T>G (p.His90Gln), NM_001354.5(AKR1C2):c.899A>C (p.Asn300Thr), NM_024105.4(ALG12):c.117del (p.Gln40fs), NM_024105.4(ALG12):c.1001del (p.Asn334fs), NM_015120.4(ALMS1):c.6436C>T (p.Arg2146Ter), NM_015120.4(ALMS1):c.2822T>A (p.Leu941Ter), NM_015120.4(ALMS1):c.6571_6574del (p.Ser2191fs), NM_015120.4(ALMS1):c.9433dup (p.Thr3145fs), NM_015120.4(ALMS1):c.1819G>T (p.Gly607Ter), NM_015120.4(ALMS1):c.9116del (p.Pro3039fs), NM_015120.4(ALMS1):c.11207C>A (p.Ser3736Ter), NM_015120.4(ALMS1):c.3575del (p.Phe1192fs), NM_015120.4(ALMS1):c.1432+2_1432+15del, NM_000478.6(ALPL):c.1133A>T (p.Asp378Val), NM_000478.6(ALPL):c.881A>C (p.Asp294Ala), NM_001135745.2(ALS2):c.553del (p.Thr185fs), NM_020919.4(ALS2):c.138del (p.Ala47fs), NM_020919.4(ALS2):c.1233T>G (p.Tyr411Ter), NM_014324.6(AMACR):c.154T>C (p.Ser52Pro), NM_001142.2(AMELX):c.14_22del (p.IIe5_Ala8delinsThr), NM_001142.2(AMELX):c.113del (p.Pro38fs), NM_001142.2(AMELX):c.378del (p.Tyr127fs), NM_001142.2(AMELX):c.431del (p.Pro144fs), NM_001142.2(AMELX):c.499del (p.Leu167fs), NM_002937.5(RNASE4):c.−17-5684A>T, NM_145868.2(ANXA11):c.119A>G (p.Asp40Gly), NM_000039.2(APOA1):c.518G>C (p.Arg173Pro), NM_000484.4(APP):c.2078A>G (p.Glu693Gly), NM_000484.4(APP):c.2075C>G (p.Ala692Gly), NM_000484.4(APP):c.2140A>G (p.Thr714Ala), NM_000484.4(APP):c.2146A>G (p.lIe716Val), NM_000484.4(APP):c.2150T>G (p.Val717Gly), NM_000484.4(APP):c.2149G>C (p.Val717Leu), NM_000485.3(APRT):c.258_261dup (p.Lys88fs), NM_000485.3(APRT):c.321+2dup, NM_000485.3(APRT):c.194A>T (p.Asp65Val), NM_000485.3(APRT):c.400+2dup, NM_000044.6(AR):c.2155T>C (p.Trp719Arg), NM_000044.6(AR):c.2299C>G (p.Pro767Ala), NM_000044.6(AR):c.321C>A (p.Tyr107Ter), NM_000044.6(AR):c.743G>T (p.Gly248Val), NM_000044.6(AR):c.1443C>G (p.Tyr481Ter), NM_000044.6(AR):c.1748T>A (p.Phe583Tyr), NM_000044.6(AR):c.1771A>T (p.Lys591Ter), NM_000044.6(AR):c.2069A>C (p.His690Pro), NM_000044.6(AR):c.2266del (p.Thr756fs), NM_000044.6(AR):c.2599G>A (p.Val867Met), NM_000044.6(AR):c.2668G>A (p.Val890Met), NM_000044.6(AR):c.2521C>T (p.Arg841Cys), NM_000044.6(AR):c.2567G>A (p.Arg856His), NM_000044.6(AR):c.2176T>C (p.Phe726Leu), NM_000044.6(AR):c.2318+1G>C, NM_017519.2(ARID1B):c.4234dup (p.Tyr1412fs), NM_007348.4(ATF6):c.797dup (p.Pro266_Asn267insTer), NM_007348.4(ATF6):c.1110dup (p.Val371fs), NM_007348.4(ATF6):c.355dup (p.Glu119Glyfs), NM_007348.4(ATF6):c.82+5G>T, NM_007348.4(ATF6):c.1187+5G>C, NM_000701.8(ATP1A1):c.311T>G (p.Leu104Arg), NM_000701.8(ATP1A1):c.995T>G (p.Val332Gly), NM_152296.5(ATP1A3):c.410C>T (p.Ser137Phe), NM_152296.5(ATP1A3):c.821T>A (p.IIe274Asn), NM_152296.5(ATP1A3):c.419A>T (p.Gln140Leu), NM_152296.5(ATP1A3):c.1003A>C (p.Thr335Pro), NM_152296.5(ATP1A3):c.2428A>T (p.IIe810Phe), NM_152296.5(ATP1A3):c.2429T>G (p.IIe810Ser), NM_152296.5(ATP1A3):c.2318A>T (p.Asn773IIe), NM_152296.5(ATP1A3):c.965T>A (p.Val322Asp), NM_152296.5(ATP1A3):c.2443G>A (p.Glu815Lys), NM_152296.5(ATP1A3):c.2431T>C (p.Ser811Pro), NM_001698.2(AUH):c.943-2A>G, NM_022893.4(BCL11A):c.295del (p.Val99fs), NM_001003800.2(BICD2):c.1636_1638del (p.Asn546del), NM_001203.3(BMPR1B):c.640C>A (p.Arg214Ser), NM_004333.6(BRAF):c.1801A>G (p.Lys601Glu), NM_004333.6(BRAF):c.1405G>C (p.Gly469Arg), NM_004333.6(BRAF):c.1781A>G (p.Asp594Gly), NM_000061.2(BTK):c.83G>A (p.Arg28His), NM_152269.5(C12orf65):c.248del (p.Val83fs), NM_001321759.2(C15orf41):c.533T>A (p.Leu178Gln), NM_001321759.2(C15orf41):c.281A>G (p.Tyr94Cys), NM_033124.5(CCDC65):c.494del (p.Glu165fs), NM_052988.5(CDK10):c.139del (p.Glu47fs), NM_001323289.2(CDKL5):c.1675C>T (p.Arg559Ter), NM_001323289.2(CDKL5):c.532C>T (p.Arg178Trp), NM_001323289.2(CDKL5):c.2635_2636del (p.Leu879fs), NM_001323289.2(CDKL5):c.2345C>A (p.Ser782Ter), NM_001323289.2(CDKL5):c.2022del (p.Phe675fs), NM_001323289.2(CDKL5):c.244del (p.Arg82fs), NM_001323289.2(CDKL5):c.659T>C (p.Leu220Pro), NM_004364.4(CEBPA):c.68del (p.Pro23fs), NM_004364.4(CEBPA):c.141del (p.Ala48fs), NM_004364.4(CEBPA):c.251A>T (p.His84Leu), NM_004364.4(CEBPA):c.115_121del (p.Pro39fs), NM_000204.4(CFI):c.162C>G (p.Cys54Trp), NM_000204.4(CFI):c.1253A>T (p.His418Leu), NM_014043.4(CHMP2B):c.618A>C (p.Gln206His), NM_017882.3(CLN6):c.200T>C (p.Leu67Pro), NM_017882.3(CLN6):c.17G>C (p.Arg6Thr), NM_001258392.3(CLPB):c.1159C>T (p.Arg387Ter), NM_001258392.3(CLPB):c.1847G>T (p.Gly616Val), NM_001258392.3(CLPB):c.1595del (p.IIe532fs), NM_001258392.3(CLPB):c.1760A>G (p.Tyr587Cys), NM_001258392.3(CLPB):c.871A>T (p.Lys291Ter), NM_001258392.3(CLPB):c.646+7323G>T, NM_001079878.2(CNGA3):c.107_110del (p.His36fs), NM_001079878.2(CNGA3):c.818C>G (p.Thr273Arg), NM_019098.4(CNGB3):c.819_826del (p.Arg274fs), NM_019098.4(CNGB3):c.1782-2A>C, NM_019098.4(CNGB3):c.1579-2A>G, NM_019098.4(CNGB3):c.702T>A (p.Cys234Ter), NM_019098.4(CNGB3):c.1635T>A (p.Tyr545Ter), NM_019098.4(CNGB3):c.904-2A>T, NM_019098.4(CNGB3):c.756C>G (p.Tyr252Ter), NM_019098.4(CNGB3):c.494-2A>T, NM_019098.4(CNGB3):c.882C>G (p.Tyr294Ter), NM_019098.4(CNGB3):c.190del (p.Glu64fs), NM_019098.4(CNGB3):c.589_590del (p.Leu197fs), NM_019098.4(CNGB3):c.2359del (p.Ser787fs), NM_019098.4(CNGB3):c.1493del (p.Leu498fs), NM_019098.4(CNGB3):c.281_284del (p.Pro94fs), NM_019098.4(CNGB3):c.1815del (p.Ala606fs), NM_019098.4(CNGB3):c.412del (p.Arg138fs), NM_000494.4(COL17A1):c.2407G>T (p.Gly803Ter), NM_000494.4(COL17A1):c.3922del (p.Ser1308fs), NM_001844.5(COL2A1):c.2671G>C (p.Gly891Arg), NM_001844.5(COL2A1):c.1957C>T (p.Arg653Ter), NM_001844.5(COL2A1):c.258C>A (p.Cys86Ter), NM_001844.5(COL2A1):c.2965C>T (p.Arg989Cys), NM_001845.6(COL4A1):c.1493G>T (p.Gly498Val), NM_001845.6(COL4A1):c.1492G>C (p.Gly498Arg), NM_000091.4(COL4A3):c.4474A>T (p.Ser1492Cys), NM_000091.4(COL4A3):c.998G>C (p.Gly333Ala), NM_000091.4(COL4A3):c.4441C>T (p.Arg1481Ter), NM_000091.4(COL4A3):c.2452G>A (p.Gly818Arg), NM_000092.4(COL4A4):c.2171del (p.Arg724fs), NM_000092.4(COL4A4):c.1389del (p.Asn464fs), NM_000092.4(COL4A4):c.4129C>T (p.Arg1377Ter), NM_000092.4(COL4A4):c.2638_2639del (p.Ala880fs), NM_000495.5(COL4A5):c.1147G>C (p.Gly383Arg), NM_000495.5(COL4A5):c.1780-1G>T, NM_000495.5(COL4A5):c.3178G>T (p.Gly1060Ter), NM_000495.5(COL4A5):c.1010G>T (p.Gly337Val), NM_000495.5(COL4A5):c.1561G>T (p.Gly521Cys), NM_000495.5(COL4A5):c.4613G>C (p.Trp1538Ser), NM_000138.4(FBN1):c.5788+5G>A, NM_015657.3(ABCA12):c.6369del (p.Val2124fs), NM_000350.3(ABCA4):c.3210_3211dup (p.Ser1071fs), NM_000350.3(ABCA4):c.6445C>T (p.Arg2149Ter), NM_000350.3(ABCA4):c.2041C>T (p.Arg681Ter), NM_003742.4(ABCB11):c.890A>G (p.Glu297Gly), NM_003500.4(ACOX2):c.207T>A (p.Tyr69Ter), NM_001101.5(ACTB):c.611C>G (p.Ala204Gly), NM_001101.5(ACTB):c.34A>C (p.Asn12His), NM_001101.5(ACTB):c.586C>A (p.Arg196Ser), NM_001101.5(ACTB):c.350A>T (p.Glu117Val), NM_001101.5(ACTB):c.802G>C (p.Gly268Arg), NM_001101.5(ACTB):c.329del (p.Leu110fs), NM_001101.5(ACTB):c.1117A>T (p.Lys373Ter), NM_001101.5(ACTB):c.269_271del (p.Phe90del), NM_001101.5(ACTB):c.587G>A (p.Arg196His), NM_001101.5(ACTB):c.193C>G (p.Leu65Val), NM_001614.5(ACTG1):c.766C>T (p.Arg256Trp), NM_001614.5(ACTG1):c.404C>T (p.Ala135Val), NM_001614.5(ACTG1):c.760C>T (p.Arg254Trp), NM_001614.5(ACTG1):c.608C>A (p.Thr203Lys), NM_001614.5(ACTG1):c.359C>T (p.Thr120IIe), NM_004302.5(ACVR1B):c.1159_1163del (p.Asp387fs), NM_198569.3(ADGRG6):c.2306T>A (p.Val769Glu), NM_198569.3(ADGRG6):c.2144dup (p.Gln716fs), NM_000027.4(AGA):c.800del (p.Leu267fs), NM_000027.4(AGA):c.216del (p.Ser72_Val73insTer), NM_000027.4(AGA):c.70del (p.Ser24fs), NM_000027.4(AGA):c.369_373del (p.His124fs), NM_000027.4(AGA):c.788del (p.IIe262_Leu263insTer), NM_000027.4(AGA):c.488G>C (p.Cys163Ser), NM_000383.4(AIRE):c.682G>T (p.Gly228Trp), NM_000383.4(AIRE):c.789del (p.Ala264fs), NM_000383.4(AIRE):c.967_979del (p.Leu323fs), NM_001139.3(ALOX12B):c.1389del (p.Phe463fs), NM_001139.3(ALOX12B):c.199A>T (p.IIe67Phe), NM_001139.3(ALOX12B):c.1562A>G (p.Tyr521Cys), NM_001165960.1(ALOXE3):c.2285C>T (p.Pro762Leu), NM_013275.6(ANKRD11):c.5317G>T (p.Glu1773Ter), NM_013275.6(ANKRD11):c.3138T>A (p.Cys1046Ter), NM_000039.2(APOA1):c.500C>G (p.Pro167Arg), NM_000039.2(APOA1):c.391A>T (p.Lys131Ter), NM_000039.2(APOA1):c.83C>G (p.Pro28Arg), NM_000039.2(APOA1):c.80C>G (p.Pro27Arg), NM_000039.2(APOA1):c.539T>A (p.Val180Glu), NM_000483.5(APOC2):c.229A>C (p.Lys77Gln), NM_000483.5(APOC2):c.122A>C (p.Lys41Thr), NM_000041.4(APOE):c.940A>C (p.Ser314Arg), NM_000485.3(APRT):c.407T>C (p.Met136Thr), NM_001195248.2(APTX):c.697A>T (p.Lys233Ter), NM_001195248.2(APTX):c.788T>G (p.Val263Gly), NM_001195248.2(APTX):c.689dup (p.Glu232fs), NM_001195248.2(APTX):c.841del (p.Ser281fs), NM_001195248.2(APTX):c.776del (p.Val259fs), NM_000045.4(ARG1):c.32T>C (p.IIe11Thr), NM_000045.4(ARG1):c.383A>G (p.Asp128Gly), NM_000045.4(ARG1):c.938del (p.Lys313fs), NM_000045.4(ARG1):c.61C>T (p.Arg21Ter), NM_000045.4(ARG1):c.466-1G>C, NM_020732.3(ARID1B):c.5968C>T (p.Arg1990Ter), NM_001278293.3(ARL6):c.92C>G (p.Thr31Arg), NM_001278293.3(ARL6):c.509T>G (p.Leu170Trp), NM_001278293.3(ARL6):c.185+1G>C, NM_025139.6(ARMC9):c.51+5G>T, NM_025139.6(ARMC9):c.1027C>A (p.Arg343Ser), NM_025139.6(ARMC9):c.1474+1G>C, NM_000487.6(ARSA):c.1283C>T (p.Pro428Leu), NM_000048.4(ASL):c.524+2T>G, NM_000048.4(ASL):c.1122dup (p.Tyr375fs), NM_000048.4(ASL):c.257A>C (p.Glu86Ala), NM_000048.4(ASL):c.94_100del (p.Arg32fs), NM_000048.4(ASL):c.857A>G (p.Gln286Arg), NM_000048.4(ASL):c.1060C>T (p.Gln354Ter), NM_000048.4(ASL):c.446+1G>A, NM_000048.4(ASL):c.544C>T (p.Arg182Ter), NM_000048.4(ASL):c.532G>A (p.Val178Met), NM_000048.4(ASL):c.1045_1057del (p.Val349fs), NM_001673.5(ASNS):c.413A>T (p.Asp138Val), NM_000049.3(ASPA):c.854A>C (p.Glu285Ala), NM_000049.3(ASPA):c.693C>A (p.Tyr231Ter), NM_030632.3(ASXL3):c.1961dup (p.Ser654_Ser655insTer), NM_030632.3(ASXL3):c.1682C>A (p.Ser561Ter), NM_030632.3(ASXL3):c.3106C>T (p.Arg1036Ter), NM_030632.3(ASXL3):c.3464C>A (p.Ser1155Ter), NM_004320.5(ATP2A1):c.1491T>G (p.Tyr497Ter), NM_000489.5(ATRX):c.6104A>T (p.Asp2035Val), NM_000489.5(ATRX):c.751A>G (p.Lys251Glu), NM_000489.5(ATRX):c.736C>T (p.Arg246Cys), NM_001701.4(BAAT):c.858C>G (p.Ser286=), NM_004656.4(BAP1):c.1717del (p.Leu573fs), NM_138761.4(BAX):c.121del (p.Glu41fs), NM_001195304.1(BBIP1):c.*18T>G, NM_024649.5(BBS1):c.1240G>T (p.Glu414Ter), NM_024649.5(BBS1):c.952-1G>C, NM_024649.5(BBS1):c.1514_1515del (p.Leu505fs), NM_024649.5(BBS1):c.223_224del (p.Leu75fs), NM_024649.5(BBS1):c.1072del (p.Tyr358fs), NM_024649.5(BBS1):c.1169T>G (p.Met390Arg), NM_024685.4(BBS10):c.999T>A (p.Cys333Ter), NM_024685.4(BBS10):c.1143T>G (p.Tyr381Ter), NM_024685.4(BBS10):c.1856_1865del (p.Lys619fs), NM_024685.4(BBS10):c.1547del (p.Thr516fs), NM_024685.4(BBS10):c.118A>T (p.Lys40Ter), NM_024685.4(BBS10):c.1024dup (p.IIe342fs), NM_024685.4(BBS10):c.1407T>G (p.Tyr469Ter), NM_024685.4(BBS10):c.1599_1602del (p.Thr534fs), NM_024685.4(BBS10):c.271dup (p.Cys91fs), NM_024685.4(BBS10):c.1044_1045del (p.Pro350fs), NM_024685.4(BBS10):c.145C>T (p.Arg49Trp), NM_024685.4(BBS10):c.273C>G (p.Cys91Trp), NM_024685.4(BBS10):c.687del (p.Val230fs), NM_024685.4(BBS10):c.1091del (p.Asn364fs), NM_024685.4(BBS10):c.39_46del (p.Ala14fs), NM_152618.3(BBS12):c.323C>G (p.Pro108Arg), NM_152618.3(BBS12):c.104C>A (p.Ser35Ter), NM_152618.3(BBS12):c.1483_1484del (p.Glu495fs), NM_152618.3(BBS12):c.1115_1116del (p.Gly371_Phe372insTer), NM_031885.4(BBS2):c.522T>A (p.Asp174Glu), NM_031885.4(BBS2):c.940del (p.IIe314fs), NM_031885.4(BBS2):c.263del (p.Gly88fs), NM_031885.4(BBS2):c.646C>T (p.Arg216Ter), NM_031885.4(BBS2):c.814C>T (p.Arg272Ter), NM_031885.4(BBS2):c.1770del (p.Phe590fs), NM_031885.4(BBS2):c.1895G>C (p.Arg632Pro), NM_033028.5(BBS4):c.220+1G>C, NM_033028.5(BBS4):c.406-2A>C, NM_033028.5(BBS4):c.638T>A (p.Leu213Ter), NM_033028.5(BBS4):c.1226del (p.Ser409fs), NM_033028.5(BBS4):c.157-2A>G, NM_033028.5(BBS4):c.1106+2T>A, NM_152384.3(BBS5):c.522+3A>G, NM_176824.3(BBS7):c.1413T>A (p.Tyr471Ter), NM_001003800.2(BICD2):c.565A>T (p.IIe189Phe), NM_001003800.2(BICD2):c.320C>T (p.Ser107Leu), NM_139343.3(BIN1):c.1723A>T (p.Lys575Ter), NM_000057.4(BLM):c.557_559del (p.Ser186_Lys187delinsTer), NM_000057.4(BLM):c.1544del (p.Asn515fs), NM_000057.4(BLM):c.213_214del (p.Ser72fs), NM_000057.4(BLM):c.320dup (p.Leu107fs), NM_000057.4(BLM):c.2258T>A (p.Leu753Ter), NM_000057.4(BLM):c.2193+1_2193+9del, NM_000057.4(BLM):c.991_995del (p.Lys331fs), NM_000057.4(BLM):c.2824-2A>T, NM_000057.4(BLM):c.4000_4004del (p.Arg1334fs), NM_000057.4(BLM):c.1479_1480del (p.Thr494fs), NM_000057.4(BLM):c.3305_3306del (p.His1102fs), NM_000057.4(BLM):c.662_665del (p.Thr221fs), NM_000057.4(BLM):c.3638del (p.Glu1213fs), NM_000057.4(BLM):c.1795del (p.Arg599fs), NM_000057.4(BLM):c.2506_2507del (p.Arg836fs), NM_000057.4(BLM):c.2098C>T (p.Gln700Ter), NM_000057.4(BLM):c.2407dup (p.Trp803fs), NM_000057.4(BLM):c.3210+2del, NM_001203.3(BMPR1B):c.975A>C (p.Lys325Asn), NM_004333.6(BRAF):c.1388T>G (p.IIe463Ser), NM_057176.3(BSND):c.1A>T (p.Met1Leu), NM_001370658.1(BTD):c.395C>G (p.Thr132Arg), NM_001370658.1(BTD):c.197T>G (p.Met66Arg), NM_001370658.1(BTD):c.623A>G (p.Asp208Gly), NM_001370658.1(BTD):c.1433dup (p.Leu478fs), NM_001370658.1(BTD):c.1312dup (p.Cys438fs), NM_001370658.1(BTD):c.1567G>C (p.Asp523His), NM_001370658.1(BTD):c.1568A>T (p.Asp523Val), NM_001370658.1(BTD):c.1556dup (p.Leu519fs), NM_001370658.1(BTD):c.1253A>G (p.Tyr418Cys), NM_001370658.1(BTD):c.1224C>A (p.Tyr408Ter), NM_001370658.1(BTD):c.1208G>C (p.Cys403Ser), NM_001370658.1(BTD):c.1154T>C (p.Leu385Pro), NM_001370658.1(BTD):c.941T>A (p.IIe314Asn), NM_001370658.1(BTD):c.1466C>G (p.Pro489Arg), NM_001370658.1(BTD):c.1451T>A (p.Met484Lys), NM_001370658.1(BTD):c.827T>G (p.Val276Gly), NM_001370658.1(BTD):c.134A>G (p.His45Arg), NM_001370658.1(BTD):c.185C>A (p.Ala62Asp), NM_001370658.1(BTD):c.776T>G (p.Leu259Trp), NM_001370658.1(BTD):c.754T>G (p.Trp252Gly), NM_001370658.1(BTD):c.635T>C (p.Phe212Ser), NM_001370658.1(BTD):c.604G>C (p.Asp202His), NM_001370658.1(BTD):c.592G>C (p.Glu198Gln), NM_001370658.1(BTD):c.124G>T (p.Val42Leu), NM_001370658.1(BTD):c.76G>T (p.Glu26Ter), NM_001370658.1(BTD):c.545A>T (p.Asn182IIe), NM_001605.2(AARS1):c.211A>T (p.Asn71Tyr), NM_020745.4(AARS2):c.464T>G (p.Leu155Arg), NM_000350.3(ABCA4):c.67-2A>G, NM_000350.3(ABCA4):c.6658C>T (p.Gln2220Ter), NM_018849.3(ABCB4):c.1633C>G (p.Arg545Gly), NM_018849.3(ABCB4):c.1712del (p.Val571fs), NM_005157.6(ABL1):c.1075T>A (p.Phe359IIe), NM_005157.6(ABL1):c.949T>A (p.Phe317IIe), NM_174917.5(ACSF3):c.593T>G (p.Met198Arg), NM_001615.4(ACTG2):c.769C>T (p.Arg257Cys), NM_001615.4(ACTG2):c.442C>A (p.Arg148Ser), NM_001615.4(ACTG2):c.119G>A (p.Arg40His), NM_018238.4(AGK):c.424-3C>G, NM_001029882.3(AHDC1):c.997del (p.Ala333fs), NM_001029882.3(AHDC1):c.1402dup (p.Cys468fs), NM 004208.4(AIFM1):c.603_605del (p.Arg201del), NM_000484.4(APP):c.2113C>G (p.Leu705Val), NM_020732.3(ARID1B):c.4377del (p.Pro1460fs), NM_020732.3(ARID1B):c.3228C>G (p.Tyr1076Ter), NM_020732.3(ARID1B):c.3345+2T>G, NM_017519.2(ARID1B):c.3650+1G>C, NM_020732.3(ARID1B):c.3737C>A (p.Ser1246Ter), NM_020732.3(ARID1B):c.4140C>G (p.Tyr1380Ter), NM_017519.2(ARID1B):c.3509del (p.Pro1170fs), NM_020732.3(ARID1B):c.3450del (p.Phe1150fs), NM_017519.2(ARID1B):c.6216_6217del (p.Leu2073_Cys2074insTer), NM_017519.2(ARID1B):c.3999T>A (p.Tyr1333Ter), NM_020732.3(ARID1B):c.5153del (p.Lys1718fs), NM_017519.2(ARID1B):c.3057_3061del (p.Lys1020fs), NM_020732.3(ARID1B):c.4009C>T (p.Arg1337Ter), NM_020732.3(ARID1B):c.4870C>T (p.Arg1624Ter), NM_152641.4(ARID2):c.3411_3412del (p.Gly1139fs), NM_152641.4(ARID2):c.4441del (p.His1481fs), NM_152641.4(ARID2):c.1028T>A (p.Leu343Ter), NM_000047.2(ARSL):c.410G>T (p.Gly137Val), NM_000047.2(ARSL):c.332G>C (p.Arg111Pro), NM_000047.2(ARSL):c.119T>G (p.IIe40Ser), NM_054012.4(ASS1):c.450 1_451del (p.Phe150fs), NM_054012.4(ASS1):c.535T>C (p.Trp179Arg), NM_054012.4(ASS1):c.1085G>T (p.Gly362Val), NM_054012.4(ASS1):c.421-2A>G, NM_015338.5(ASXL1):c.3083C>A (p.Ser1028Ter), NM_001164603.1(ASXL1):c.217A>T (p.Lys73Ter), NM_000701.8(ATP1A1):c.2432A>C (p.Asp811Ala), NM_000701.8(ATP1A1):c.1798C>A (p.Pro600Thr), NM_000701.8(ATP1A1):c.143T>G (p.Leu48Arg), NM_000701.8(ATP1A1):c.1798C>G (p.Pro600Ala), NM_005603.6(ATP8B1):c.1982T>C (p.IIe661Thr), NM_004281.3(BAG3):c.1034_1038del (p.Glu345fs), NM_004281.3(BAG3):c.1292dup (p.Val432fs), NM_001256447.2(BCAP31):c.341+2T>G, NM_004333.6(BRAF):c.1408_1410del (p.Thr470del), NM_004333.6(BRAF):c.1741A>G (p.Asn581Asp), NM_004333.6(BRAF):c.1914T>G (p.Asp638Glu), NM_004333.6(BRAF):c.770A>G (p.Gln257Arg), NM_004333.6(BRAF):c.735A>C (p.Leu245Phe), NM_004333.6(BRAF):c.1722C>G (p.His574Gln), NM_004333.6(BRAF):c.736G>C (p.Ala246Pro), NM_004333.6(BRAF):c.1787G>T (p.Gly596Val), NM_004333.6(BRAF):c.1789C>G (p.Leu597Val), NM_004333.6(BRAF):c.1785T>G (p.Phe595Leu), NM_004333.6(BRAF):c.735A>T (p.Leu245Phe), NM_004333.6(BRAF):c.1399T>G (p.Ser467Ala), NM_004333.6(BRAF):c.730A>C (p.Thr244Pro), NM_004333.6(BRAF):c.1403T>C (p.Phe468Ser), NM_001519.4(BRF1):c.875C>A (p.Pro292His), NM_032667.6(BSCL2):c.793C>T (p.Arg265Ter), NM_032667.6(BSCL2):c.263A>G (p.Asn88Ser), NM_152269.5(C12orf65):c.210del (p.Gly72fs), NM_152269.5(C12orf65):c.96_99dup (p.Pro34fs), NM_001212.4(C1QBP):c.562_564del (p.Tyr188del), NM_000587.4(C7):c.281-1G>T, NM_000587.4(C7):c.1458T>A (p.Cys486Ter), NM_000587.4(C7):c.2184T>A (p.Cys728Ter), NM_000587.4(C7):c.317del (p.Asp106fs), NM_000066.4(C8B):c.1282C>T (p.Arg428Ter), NM_000066.4(C8B):c.1041_1047dup (p.Leu350fs), NM_000066.4(C8B):c.605del (p.Pro202fs), NM_000066.4(C8B):c.336del (p.Asn113fs), NM_177965.4(C8orf37):c.156-2A>G, NM_001737.5(C9):c.355T>G (p.Cys119Gly), NM_001737.5(C9):c.577del (p.Tyr193fs), NM_001174051.3(CACNA2D2):c.3158T>C (p.Leu1053Pro), NM_001174051.3(CACNA2D2):c.1295del (p.Asn432fs), NM_001013838.3(CARMIL2):c.490dup (p.Ala164fs), NM_001013838.3(CARMIL2):c.871+1 G>T, NM_024537.4(CARS2):c.649_651del (p.Glu217del), NM_001232.3(CASQ2):c.546del (p.Phe182fs), NM_053054.4(CATSPER1):c.539dup (p.His182fs), NM_053054.4(CATSPER1):c.944_948dup (p.Asp317fs), NM_000071.2(CBS):c.919G>A (p.Gly307Ser), NM_000071.2(CBS):c.833T>C (p.IIe278Thr), NM_001080522.2(CC2D2A):c.2683C>T (p.Gln895Ter), NM_213607.3(CCDCl03):c.383dup (p.Pro129fs), NM_001364171.2(CCDCl14):c.1050del (p.His350fs), NM_145045.5(CCDCl51):c.925G>T (p.Glu309Ter), NM_145045.5(CCDCl51):c.1256C>A (p.Ser419Ter), NM_017950.4(CCDC40):c.248del (p.Ala83fs), NM_031443.3(CCM2):c.593T>G (p.Leu198Arg), NM_021147.5(CCNO):c.926del (p.Pro309fs), NM_021147.5(CCNO):c.263_267dup (p.Val90fs), NM_001178098.2(CD19):c.1464del (p.Ser489fs), NM_022124.6(CDH23):c.5237G>A (p.Arg1746Gln), NM_201548.5(CERKL):c.481+2T>G, NM_021254.4(CFAP298):c.792_795del (p.Ala263_Tyr264insTer), NM_021254.4(CFAP298):c.292C>T (p.Arg98Ter), NM_030787.3(CFHR5):c.678del (p.Glu226fs), NM_017780.4(CHD7):c.8962dup (p.Asp2988fs), NM_017780.4(CHD7):c.5405-2A>G, NM_017780.4(CHD7):c.7940_7941dup (p.Pro2648fs), NM_017780.4(CHD7):c.5752dup (p.Thr1918fs), NM_017780.4(CHD7):c.5238C>G (p.Tyr1746Ter), NM_017780.4(CHD7):c.1488dup (p.Pro497fs), NM_017780.4(CHD7):c.3071dup (p.Leu1025fs), NM_017780.4(CHD7):c.3202-3T>G, NM_017780.4(CHD7):c.3768C>G (p.Tyr1256Ter), NM_017780.4(CHD7):c.5211-1G>C, NM_017780.4(CHD7):c.5405-18C>A, NM_017780.4(CHD7):c.6446del (p.Gly2149fs), NM_017780.4(CHD7):c.5101del (p.Gln1701fs), NM_017780.4(CHD7):c.4835del (p.Asn1612fs), NM_017780.4(CHD7):c.1058del (p.Phe353fs), NM_017780.4(CHD7):c.3770T>G (p.Leu1257Arg), NM_017780.4(CHD7):c.4634del (p.Ala1544_Leu1545insTer), NM_017780.4(CHD7):c.3937del (p.Ser1313fs), NM_017780.4(CHD7):c.7663del (p.Arg2555fs), NM_017780.4(CHD7):c.2990del (p.Leu997fs), NM_017780.4(CHD7):c.3209del (p.Val1070fs), NM_017780.4(CHD7):c.5235_5236dup (p.Tyr1746fs), NM_017780.4(CHD7):c.8730_8731del (p.Pro2911fs), NM_017780.4(CHD7):c.3082A>G (p.IIe1028Val), NM_017780.4(CHD7):c.3205C>T (p.Arg1069Ter), NM_017780.4(CHD7):c.6157C>T (p.Arg2053Ter), NM_017780.4(CHD7):c.5405-7G>A, NM_017780.4(CHD7):c.6079C>T (p.Arg2027Ter), NM_017780.4(CHD7):c.4480C>T (p.Arg1494Ter), NM_017780.4(CHD7):c.4393C>T (p.Arg1465Ter), NM_017780.4(CHD7):c.1480C>T (p.Arg494Ter), NM_017780.4(CHD7):c.7891C>T (p.Arg2631Ter), NM_017780.4(CHD7):c.7252C>T (p.Arg2418Ter), NM_017780.4(CHD7):c.5833C>T (p.Arg1945Ter), NM_017780.4(CHD7):c.3106C>T (p.Arg1036Ter), NM_017780.4(CHD7):c.2959C>T (p.Arg987Ter), NM_017780.4(CHD7):c.3655C>T (p.Arg1219Ter), NM_017780.4(CHD7):c.6114_6120del (p.Leu2039fs), NM_017780.4(CHD7):c.2905_2906del (p.Arg969fs), NM_017780.4(CHD7):c.2836-2A>T, NM_017780.4(CHD7):c.469C>T (p.Arg157Ter), NM_000390.4(CHM):c.1609+2dup, NM_000390.4(CHM):c.280del (p.Thr94fs), NM_000390.4(CHM):c.715C>T (p.Arg239Ter), NM_000390.4(CHM):c.877C>T (p.Arg293Ter), NM_005199.5(CHRNG):c.753_754del (p.Val253fs), NM_013246.3(CLCF1):c.590G>T (p.Arg197Leu), NM_013246.3(CLCF1):c.321C>A (p.Tyr107Ter), NM_001830.4(CLCN4):c.1630G>C (p.Gly544Arg), NM_001830.4(CLCN4):c.662T>C (p.Leu221Pro), NM_001830.4(CLCN4):c.635T>G (p.Val212Gly), NM_000086.2(CLN3):c.883G>A (p.Glu295Lys), NM_006493.4(CLN5):c.371del (p.Ser124fs), NM_006493.4(CLN5):c.78G>A (p.Trp26Ter), NM_017882.2(CLN6):c.316dupC (p.Arg106Profs), NM_017882.3(CLN6):c.250T>A (p.Tyr84Asn), NM_017882.3(CLN6):c.214G>T (p.Glu72Ter), NM_018941.3(CLN8):c.181_183del (p.Lys61del), NM_018941.3(CLN8):c.263del (p.Asp88fs), NM_018941.3(CLN8):c.47del (p.Leu16fs), NM_018941.3(CLN8):c.792C>G (p.Asn264Lys), NM_018941.3(CLN8):c.70C>G (p.Arg24Gly), NM_153603.4(COG7):c.1476-1G>T, NM_153603.4(COG7):c.169+4A>C, NM_000089.3(COL1A2):c.739G>T (p.Gly247Cys), NM_000089.3(COL1A2):c.2958del (p.Val987fs), NM_000090.3(COL3A1):c.2642del (p.Pro881fs), NM_000090.3(COL3A1):c.3325C>T (p.Arg1109Ter), NM_000090.3(COL3A1):c.997-1G>C (p.Gly333_Lys350del+), NM_000090.3(COL3A1):c.2221G>T (p.Gly741Cys), NM_015697.8(COQ2):c.545T>G (p.Met182Arg), NM_015697.8(COQ2):c.590G>A (p.Arg197His), NM_000350.3(ABCA4):c.1819G>C (p.Gly607Arg), NM_000350.3(ABCA4):c.5899-1G>T, NM_000350.3(ABCA4):c.2888del (p.Gly963fs), NM_000350.3(ABCA4):c.2616_2617del (p.Phe873fs), NM_000350.3(ABCA4):c.1804C>T (p.Arg602Trp), NM_000350.3(ABCA4):c.4539+1G>T, NM_000350.3(ABCA4):c.6729+5_6729+19del, NM_000350.3(ABCA4):c.3093del (p.Gly1032fs), NM_000350.3(ABCA4):c.5819T>C (p.Leu1940Pro), NM_000350.3(ABCA4):c.2894A>G (p.Asn965Ser), NM_000017.4(ACADS):c.593_594del (p.Phe198fs), NM_000017.4(ACADS):c.1031del (p.Glu344fs), NM_000017.4(ACADS):c.125_135del (p.Leu42fs), NM_000017.4(ACADS):c.682_683del (p.Glu228fs), NM_001609.3(ACADSB):c.303+3A>G, NM_000019.4(ACAT1):c.1006-2A>C, NM_000019.4(ACAT1):c.1163+2T>C, NM_000019.4(ACAT1):c.2T>A (p.Met1Lys), NM_000019.4(ACAT1):c.1083dup (p.Ala362fs), NM_000019.4(ACAT1):c.149del (p.Thr50fs), NM_000019.4(ACAT1):c.444_445del (p.Met148fs), NM_001100.3(ACTA1):c.668T>C (p.Leu223Pro), NM_001100.3(ACTA1):c.881A>T (p.Asp294Val), NM_001079858.3(ADGRG2):c.1545dup (p.Glu516Ter), NM_006412.4(AGPAT2):c.493-1G>C, NM_006412.4(AGPAT2):c.643A>T (p.Lys215Ter), NM_006412.4(AGPAT2):c.183-2A>G, NM_006412.4(AGPAT2):c.661+2T>G, NM_006412.4(AGPAT2):c.377dup (p.Pro128fs), NM_006412.4(AGPAT2):c.755_763del (p.Met252_Thr254del), NM_006412.4(AGPAT2):c.538del (p.Asp180fs), NM_198576.4(AGRN):c.1362dup (p.Ser455fs), NM_198576.4(AGRN):c.314A>T (p.Asn105IIe), NM_198576.4(AGRN):c.5179G>T (p.Val1727Phe), NM_198576.4(AGRN):c.5125G>C (p.Gly1709Arg), NM_004208.4(AIFM1):c.1478A>T (p.Glu493Val), NM_014336.5(AIPL1):c.1053_1064del (p.Ala352_Pro355del), NM_005163.2(AKT1):c.1303A>C (p.Thr435Pro), NM_002860.4(ALDH18A1):c.413G>T (p.Arg138Leu), NM_002860.4(ALDH18A1):c.741del (p.Asp247fs), NM_002860.4(ALDH18A1):c.2131del (p.Leu711fs), NM_003748.4(ALDH4A1):c.1560dup (p.Gly521fs), NM_019109.4(ALG1):c.1025A>C (p.Gln342Pro), NM_019109.4(ALG1):c.1187+1G>A, NM_019109.4(ALG1):c.1188-2A>G, NM_019109.4(ALG1):c.15C>A (p.Cys5Ter), NM_001004127.3(ALG11):c.836A>C (p.Tyr279Ser), NM_001004127.3(ALG11):c.953A>C (p.Gln318Pro), NM_033087.4(ALG2):c.393G>T (p.Lys131Asn), NM_033087.4(ALG2):c.1040del (p.Gly347fs), NM_005787.6(ALG3):c.1037A>G (p.Asn346Ser), NM_005787.6(ALG3):c.350G>C (p.Arg117Pro), NM_005787.6(ALG3):c.470T>A (p.Met157Lys), NM_013339.4(ALG6):c.680+2T>G, NM_013339.4(ALG6):c.171T>A (p.Tyr57Ter), NM_024079.5(ALG8):c.139A>C (p.Thr47Pro), NM_024079.5(ALG8):c.413del (p.Thr138fs), NM_054027.6(ANKH):c.1001T>G (p.Leu334Arg), NM_054027.6(ANKH):c.1124_1126del (p.Ser375del), NM_000040.3(APOC3):c.55C>T (p.Arg19Ter), NM_001940.4(ATN1):c.3160C>A (p.His1054Asn), NM_170665.4(ATP2A2):c.2300A>G (p.Asn767Ser), NM_012463.4(ATP6VOA2):c.353_354del (p.Leu118fs), NM_012463.4(ATP6VOA2):c.840del (p.Glu281fs), NM_012463.4(ATP6VOA2):c.1929del (p.Gln645fs), NM_000052.7(ATP7A):c.1537G>T (p.Glu513Ter), NM_000052.7(ATP7A):c.3911A>G (p.Asn1304Ser), NM_000055.4(BCHE):c.495_498del (p.Arg166fs), NM_000055.4(BCHE):c.206_207del (p.Leu69fs), NM_001123383.1(BCOR):c.4288_4291del (p.Glu1430fs), NM_001724.5(BPGM):c.61del (p.Arg21fs), NM_004333.6(BRAF):c.1782T>A (p.Asp594Glu), NM_032667.6(BSCL2):c.823C>T (p.Arg275Ter), NM_032667.6(BSCL2):c.325dup (p.Thr109fs), NM_001122955.3(BSCL2):c.826G>C (p.Ala276Pro), NM_032667.6(BSCL2):c.672-3C>G, NM_032667.6(BSCL2):c.782dup (p.IIe262fs), NM_032667.6(BSCL2):c.210C>G (p.Tyr70Ter), NM_032667.6(BSCL2):c.315_316del (p.Tyr106fs), NM_032667.6(BSCL2):c.636del (p.Tyr213fs), NM_032667.6(BSCL2):c.672-2A>C, NM_032667.6(BSCL2):c.154_155dup (p.Tyr53fs), NM_032667.6(BSCL2):c.652_662del (p.Ala218fs), NM_032667.6(BSCL2):c.317_321del (p.Tyr106fs), NM_000719.7(CACNA1C):c.1204G>A (p.Gly402Ser), NM_005183.4(CACNA1F):c.1218del (p.Trp407fs), NM_005183.4(CACNA1F):c.1538_1542del (p.Arg513fs), NM_005183.4(CACNA1F):c.3341_3342del (p.Ser1114fs), NM_032357.4(CCDC115):c.92T>C (p.Leu31Ser), NM_001080414.4(CCDC88C):c.5841_5842del (p.Glu1949fs), NM_138477.4(CDAN1):c.1117_1119del (p.Val373del), NM_022124.6(CDH23):c.6202A>C (p.Thr2068Pro), NM_022124.6(CDH23):c.719C>T (p.Pro240Leu), NM_022124.6(CDH23):c.4877A>C (p.Asp1626Ala), NM_022124.6(CDH23):c.5369-1G>T, NM_022124.6(CDH23):c.5147A>C (p.Gln1716Pro), NM_022124.6(CDH23):c.6667del (p.Leu2223fs), NM_022124.6(CDH23):c.9129del (p.Asn3044fs), NM_022124.6(CDH23):c.683A>T (p.Asp228Val), NM_022124.6(CDH23):c.2132_2136del (p.Tyr711fs), NM_022124.6(CDH23):c.6050-9G>A, NM_022124.6(CDH23):c.4309C>T (p.Arg1437Ter), NM_003718.5(CDK13):c.2149G>A (p.Gly717Arg), NM_001039213.4(CEACAM16):c.418A>C (p.Thr140Pro), NM_033440.3(CELA2A):c.253C>A (p.Leu85Met), NM_033440.3(CELA2A):c.639+1G>C, NM_007186.6(CEP250):c.3337A>T (p.Lys1113Ter), NM 001330691.3(CEP78):c.633del (p.Trp212fs), NM_201548.5(CERKL):c.769C>T (p.Arg257Ter), NM_000492.3(CFTR):c.1766+5G>T, NM_000492.3(CFTR):c.2490+1G>A, NM_000492.3(CFTR):c.2012del (p.Ser670_Leu671 insTer), NM_000492.3(CFTR):c.2909G>A (p.Gly970Asp), NM_000492.3(CFTR):c.1792_1798del (p.Lys598fs), NM_000492.3(CFTR):c.274G>A (p.Glu92Lys), NM_000492.3(CFTR):c.2551C>T (p.Arg851Ter), NM_000492.3(CFTR):c.1654C>T (p.Gln552Ter), NM_000492.3(CFTR):c.2195T>G (p.Leu732Ter), NM_000492.3(CFTR):c.1393-2A>G, NM_000492.3(CFTR):c.3873+2T>C, NM_000492.3(CFTR):c.580-1G>T, NM_000492.3(CFTR):c.579+5G>A, NM_000492.3(CFTR):c.3230T>C (p.Leu1077Pro), NM_000492.3(CFTR):c.2780T>C (p.Leu927Pro), NM_000492.3(CFTR):c.825C>G (p.Tyr275Ter), NM_000492.3(CFTR):c.409del (p.Leu137fs), NM_000492.3(CFTR):c.1365_1366del (p.Val456fs), NM_000492.3(CFTR):c.1373del (p.Gly458fs), NM_000492.3(CFTR):c.2215del (p.Val739fs), NM_000492.3(CFTR):c.273+3A>C, NM_000492.3(CFTR):c.3659delC, NM_000492.3(CFTR):c.3532_3535dup (p.Thr1179fs), NM_000492.3(CFTR):c.3700A>G (p.IIe1234Val), NM_000492.3(CFTR):c.3717+4A>G, NM_000492.3(CFTR):c.1826A>G (p.His609Arg), NM_000492.3(CFTR):c.169T>G (p.Trp57Gly), NM_000492.3(CFTR):c.1001G>T (p.Arg334Leu), NM_000492.3(CFTR):c.274-2A>G, NM_000492.3(CFTR):c.164+1G>T, NM_000492.3(CFTR):c.1986_1989del (p.Thr663fs), NM_000492.3(CFTR):c.1530_1531del (p.Ser511fs), NM_000492.3(CFTR):c.531del (p.IIe177fs), NM_000492.3(CFTR):c.3691del (p.Ser1231fs), NM_000492.3(CFTR):c.2547C>A (p.Tyr849Ter), NM_000492.3(CFTR):c.1466C>A (p.Ser489Ter), NM_000492.3(CFTR):c.828C>A (p.Cys276Ter), NM_000492.3(CFTR):c.366T>A (p.Tyr122Ter), NM_000492.3(CFTR):c.305T>G (p.Leu102Arg), NM_000492.3(CFTR):c.79G>T (p.Gly27Ter), NM_000492.3(CFTR):c.4124A>C (p.His1375Pro), NM_000492.3(CFTR):c.1211delG, NM_000492.3(CFTR):c.4028del (p.Gly1343fs), NM_000492.3(CFTR):c.413_415dup (p.Leu138dup), NM_000492.3(CFTR):c.3486_3487del (p.Val1163fs), NM_000492.3(CFTR):c.409_412del (p.Leu137fs), NM_000492.3(CFTR):c.1081del (p.Trp361fs), NM_000492.3(CFTR):c.416A>G (p.His139Arg), NM_000492.3(CFTR):c.454A>G (p.Met152Val), NM_000492.3(CFTR):c.675T>A (p.Cys225Ter), NM_000492.3(CFTR):c.1505T>C (p.IIe502Thr), NM_000492.3(CFTR):c.164+2T>C, NM_000492.3(CFTR):c.164+4dup, NM_000492.3(CFTR):c.3844T>G (p.Trp1282Gly), NM_000492.3(CFTR):c.3292T>C (p.Trp1098Arg), NM_000492.3(CFTR):c.1538A>G (p.Asp513Gly), NM_000492.3(CFTR):c.1585-2A>G, NM_000492.3(CFTR):c.1736A>G (p.Asp579Gly), NM_000492.3(CFTR):c.3017C>A (p.Ala1006Glu), NM_000492.3(CFTR):c.3039dup (p.Tyr1014fs), NM_000492.3(CFTR):c.3719T>G (p.Val1240Gly), NM_000492.3(CFTR):c.326A>G (p.Tyr109Cys), NM_000492.3(CFTR):c.3848G>T (p.Arg1283Met), NM_000492.3(CFTR):c.3971T>C (p.Leu1324Pro), NM_000492.3(CFTR):c.3891dup (p.Gly1298fs), NM_000492.3(CFTR):c.2464G>T (p.Glu822Ter), NM_000492.3(CFTR):c.2601dup (p.Val868fs), NM_000492.3(CFTR):c.2658-1G>C, NM_000492.3(CFTR):c.2763_2764dup (p.Val922fs), NM_000492.3(CFTR):c.2989-2A>G, NM_000492.3(CFTR):c.3367+2T>A, NM_001089.3(ABCA3):c.875A>T (p.Glu292Val), NM_000392.5(ABCC2):c.3517A>T (p.IIe1173Phe), NM_000392.5(ABCC2):c.3399_3400del (p.Tyr1134fs), NM_001287174.2(ABCC8):c.221G>A (p.Arg74Gln), NM_001287174.2(ABCC8):c.394T>C (p.Phe132Leu), NM_005691.3(ABCC9):c.3460C>T (p.Arg1154Trp), NM_005691.3(ABCC9):c.3461G>A (p.Arg1154Gln), NM_001082486.2(ACD):c.250_252del (p.Lys84del), NM_001082486.2(ACD):c.1213C>A (p.Pro405Thr), NM_002036.4(ACKR1):c.286_299del (p.Trp96fs), NM_014244.5(ADAMTS2):c.2458-6_2458del, NM_014244.5(ADAMTS2):c.673C>T (p.Gln225Ter), NM_019032.5(ADAMTSL4):c.239del (p.Pro80fs), NM_183357.2(ADCY5):c.3086T>A (p.Met1029Lys), NM_001029882.3(AHDC1):c.2547del (p.Ser850fs), NM_001029882.3(AHDC1):c.2898del (p.Tyr967fs), NM_031418.4(ANO3):c.1480A>T (p.Arg494Trp), NM_000486.5(AQP2):c.170A>C (p.Gln57Pro), NM_000486.5(AQP2):c.559C>T (p.Arg187Cys), NM_001651.4(AQP5):c.529A>T (p.IIe177Phe), NM_173728.4(ARHGEF15):c.709_723del (p.Val237_Ala241del), NM_015185.3(ARHGEF9):c.164G>C (p.Gly55Ala), NM_020732.3(ARID1B):c.6357del (p.Asp2121fs), NM_152296.5(ATP1A3):c.829G>A (p.Glu277Lys), NM_152296.5(ATP1A3):c.2338T>C (p.Phe780Leu), NM_152296.5(ATP1A3):c.2273T>G (p.IIe758Ser), NM_152296.5(ATP1A3):c.821T>C (p.IIe274Thr), NM_152296.5(ATP1A3):c.1838C>T (p.Thr613Met), NM_004281.3(BAG3):c.925C>T (p.Arg309Ter), NM_004281.3(BAG3):c.699C>A (p.Tyr233Ter), NM_001365308.1(BMPER):c.1638T>A (p.Cys546Ter), NM_001733.7(C1R):c.1073G>T (p.Cys358Phe), NM_001733.7(C1R):c.899T>C (p.Leu300Pro), NM_001733.7(C1R):c.869A>G (p.Asp290Gly), NM_001733.7(C1R):c.902G>C (p.Arg301Pro), NM_001733.7(C1R):c.927C>G (p.Cys309Trp), NM_001733.7(C1R):c.1113C>G (p.Cys371Trp), NM_001733.7(C1R):c.905A>G (p.Tyr302Cys), NM_001733.7(C1R):c.1012T>C (p.Cys338Arg), NM_001733.7(C1R):c.1303T>C (p.Trp435Arg), NM_001733.7(C1R):c.1092G>C (p.Trp364Cys), NM_001734.5(C1S):c.880T>C (p.Cys294Arg), NM_001734.5(C1S):c.945_947del (p.Asp315_Val316delinsGlu), NM_001174051.3(CACNA2D2):c.485_486del (p.Tyr161_Tyr162insTer), NM_001159773.2(CANT1):c.734del (p.Pro245fs), NM_033337.2(CAV3):c.99C>G (p.Asn33Lys), NM_033337.2(CAV3):c.136G>A (p.Ala46Thr), NM_033337.2(CAV3):c.80G>A (p.Arg27Gln), NM_001080522.2(CC2D2A):c.4179+1del, NM_001793.6(CDH3):c.965A>T (p.Asn322IIe), NM_001793.6(CDH3):c.830del (p.Gly277fs), NM_001323289.2(CDKL5):c.578A>G (p.Asp193Gly), NM_001323289.2(CDKL5):c.533G>C (p.Arg178Pro), NM_001323289.2(CDKL5):c.1082dup (p.Ala362fs), NM_001323289.2(CDKL5):c.747dup (p.Pro250fs), NM_001323289.2(CDKL5):c.1797dup (p.Ser600fs), NM_001323289.2(CDKL5):c.1784dup (p.Leu596fs), NM_001323289.2(CDKL5):c.2016dup (p.Ser673fs), NM_001323289.2(CDKL5):c.2016del (p.Ser673fs), NM_001323289.2(CDKL5):c.2066del (p.Pro689fs), NM_001323289.2(CDKL5):c.2504del (p.Pro835fs), NM_001323289.2(CDKL5):c.39del (p.Phe13fs), NM_001323289.2(CDKL5):c.884del (p.Pro295fs), NM_001323289.2(CDKL5):c.1684_1687del (p.Thr562fs), NM_001323289.2(CDKL5):c.1147_1151del (p.Thr383fs), NM_001323289.2(CDKL5):c.2593C>T (p.Gln865Ter), NM_001323289.2(CDKL5):c.513C>A (p.Tyr171Ter), NM_001323289.2(CDKL5):c.2572del (p.Arg858fs), NM_001323289.2(CDKL5):c.125A>G (p.Lys42Arg), NM_003159.2(CDKL5):c.65dupG, NM_001323289.2(CDKL5):c.978-2A>G, NM_001323289.2(CDKL5):c.464-2A>G, NM_001323289.2(CDKL5):c.404-1G>T, NM_001323289.2(CDKL5):c.64+2del, NM_001323289.2(CDKL5):c.215T>C (p.IIe72Thr), NM_001822.5(CHN1):c.378T>G (p.IIe126Met), NM_130468.3(CHST14):c.638G>C (p.Arg213Pro), NM_001127898.4(CLCN5):c.989G>T (p.Gly330Val), NM_001127898.4(CLCN5):c.1396G>C (p.Asp466His), NM_001127898.4(CLCN5):c.809T>G (p.Leu270Arg), NM_001127898.4(CLCN5):c.2119C>T (p.Arg707Ter), NM_005076.5(CNTN2):c.504del (p.Trp168fs), NM_015386.3(COG4):c.1840G>T (p.Glu614Ter), NM_000088.3(COL1A1):c.3040C>T (p.Arg1014Cys), NM_000088.3(COL1A1):c.2089C>T (p.Arg697Ter), NM_000088.3(COL1A1):c.1243C>T (p.Arg415Ter), NM_000088.3(COL1A1):c.1299+1G>A, NM_000088.3(COL1A1):c.2362G>A (p.Gly788Ser), NM_000088.3(COL1A1):c.994G>A (p.Gly332Arg), NM_000089.3(COL1A2):c.1127G>T (p.Gly376Val), NM_000089.3(COL1A2):c.279+2T>C, NM_000089.3(COL1A2):c.767G>T (p.Gly256Val), NM_000089.3(COL1A2):c.577G>A (p.Gly193Ser), NM_000089.3(COL1A2):c.838G>A (p.Gly280Ser), NM_000090.3(COL3A1):c.1610delG, NM_000090.3(COL3A1):c.2022+2T>C (p.Gly660_Lys674del), NM_000090.3(COL3A1):c.2870G>T (p.Gly957Val), NM_000090.3(COL3A1):c.800G>T (p.Gly267Val), NM_000090.3(COL3A1):c.3149G>T (p.Gly1050Val), NM_000090.3(COL3A1):c.3230G>T (p.Gly1077Val), NM_000090.3(COL3A1):c.575G>T (p.Gly192Val), NM_000090.3(COL3A1):c.620G>T (p.Gly207Val), NM_000090.3(COL3A1):c.629G>T (p.Gly210Val), NM_000090.3(COL3A1):c.755G>T (p.Gly252Val), NM_000090.3(COL3A1):c.754G>C (p.Gly252Arg), NM_000090.3(COL3A1):c.746G>T (p.Gly249Val), NM_000090.3(COL3A1):c.728G>T (p.Gly243Val), NM_000090.3(COL3A1):c.718G>C (p.Gly240Arg), NM_000090.3(COL3A1):c.690+2T>G (p.Gly213_Asp230del), NM_000090.3(COL3A1):c.682G>C (p.Gly228Arg), NM_000090.3(COL3A1):c.674G>T (p.Gly225Val), NM_000090.3(COL3A1):c.665G>T (p.Gly222Val), NM_000090.3(COL3A1):c.656G>C (p.Gly219Ala), NM_000090.3(COL3A1):c.655G>T (p.Gly219Cys), NM_000090.3(COL3A1):c.764G>T (p.Gly255Val), NM_000090.3(COL3A1):c.547G>C (p.Gly183Arg), NM_000090.3(COL3A1):c.996+2T>A (p.Gly318_Pro332del), NM_000090.3(COL3A1):c.1347+1G>T (p.Arg449_Gly450insValSerPheThrAlaThrAspLeu+) (“ValSerPheThrAlaThrAspLeu” disclosed as SEQ ID NO: 606), NM_000090.3(COL3A1):c.1330G>C (p.Gly444Arg), NM_000090.3(COL3A1):c.1294-3T>G (p.Gly432_Arg449del), NM_000090.3(COL3A1):c.898-1G>C (p.Gly300_Ala317del), NM_000090.3(COL3A1):c.1240G>T (p.Gly414Cys), NM_000090.3(COL3A1):c.899G>T (p.Gly300Val), NM_000090.3(COL3A1):c.962G>T (p.Gly321Val), NM_000090.3(COL3A1):c.951+4A>T (p.Gly300_Ala317del), NM_000090.3(COL3A1):c.951+3G>T (p.Gly300_Ala317del), NM_000090.3(COL3A1):c.944G>C (p.Gly315Ala), NM_000090.3(COL3A1):c.1231G>C (p.Gly411Arg), NM_000090.3(COL3A1):c.583G>C (p.Gly195Arg), NM_000090.3(COL3A1):c.582+6T>A (p.Gly177_Pro194del), NM_000090.3(COL3A1):c.582+5G>T (p.Gly177_Pro194del), NM_000090.3(COL3A1):c.582+2dup, NM_000090.3(COL3A1):c.619G>T (p.Gly207Trp), NM_000090.3(COL3A1):c.582+1G>C (p.Gly177_Pro194del), NM_000090.3(COL3A1):c.565G>C (p.Gly189Arg), NM_000090.3(COL3A1):c.548G>C (p.Gly183Ala), NM_000090.3(COL3A1):c.547G>T (p.Gly183Cys), NM_000090.3(COL3A1):c.601G>C (p.Gly201Arg), NM_000090.3(COL3A1):c.1194+1G>C (p.Gly384_Met398del), NM_000090.3(COL3A1):c.1150-1G>C (p.Gly384_Met398del), NM_000090.3(COL3A1):c.1115G>T (p.Gly372Val), NM_000090.3(COL3A1):c.3275G>T (p.Gly1092Val), NM_000090.3(COL3A1):c.3417+1G>C (p.Gly1122 Arg1139del+), NM_000090.3(COL3A1):c.3257G>T (p.Gly1086Val), NM_000090.3(COL3A1):c.3203G>T (p.Gly1068Val), NM_000090.3(COL3A1):c.3176G>T (p.Gly1059Val), NM_000090.3(COL3A1):c.3157G>C (p.Gly1053Arg), NM_000090.3(COL3A1):c.3417+1G>T (p.Gly1122 Arg1139del+), NM_000090.3(COL3A1):c.3418-2A>T (p.Arg1139_Gly1140insVSSTERYYRSTCFRCLHFRKIFWHCDVMILSL) (“VSSTERYYRSTCFRCLHFRKIFWHCDVMILSL” disclosed as SEQ ID NO: 607), NM_000090.3(COL3A1):c.3535G>C (p.Gly1179Arg), NM_000090.3(COL3A1):c.3293G>T (p.Gly1098Val), NM_000090.3(COL3A1):c.1915G>C (p.Gly639Arg), NM_000090.3(COL3A1):c.1906G>C (p.Gly636Arg), NM_000090.3(COL3A1):c.2078G>C (p.Gly693Ala), NM_000090.3(COL3A1):c.3364-2A>C (p.Gly1122 Arg1139del), NM_000090.3(COL3A1):c.3347G>T (p.Gly1116Val), NM_000090.3(COL3A1):c.3536G>T (p.Gly1179Val), NM_000090.3(COL3A1):c.3518G>T (p.Gly1173Val), NM_000090.3(COL3A1):c.1925G>T (p.Gly642Val), NM_000090.3(COL3A1):c.3122G>T (p.Gly1041Val), NM_000090.3(COL3A1):c.3104G>T (p.Gly1035Val), NM_000090.3(COL3A1):c.3093+2T>G (p.Gly1014_Lys1031del), NM_000090.3(COL3A1):c.2815G>T (p.Gly939Cys), NM_000090.3(COL3A1):c.2806G>C (p.Gly936Arg), NM_020686.6(ABAT):c.1433T>C (p.Leu478Pro), NM_005502.4(ABCA1):c.5192C>G (p.Ser1731Cys), NM_001171.5(ABCC6):c.450dup (p.Ala151fs), NM_001171.5(ABCC6):c.2787+1G>T, NM_001171.5(ABCC6):c.4216C>A (p.Gln1406Lys), NM_001171.5(ABCC6):c.3940C>T (p.Arg1314Trp), NM_001171.5(ABCC6):c.3904G>A (p.Gly1302Arg), NM_001171.5(ABCC6):c.1552C>T (p.Arg518Ter), NM_001171.5(ABCC6):c.1553G>A (p.Arg518Gln), NM_001287174.2(ABCC8):c.62T>A (p.Val21Asp), NM_001287174.2(ABCC8):c.3992-9G>A, NM_001287174.2(ABCC8):c.4163_4165del (p.Phe1388del), NM_005159.5(ACTC1):c.997G>C (p.Ala333Pro), NM_005159.5(ACTC1):c.496C>G (p.Pro166Ala), NM_016188.5(ACTL6B):c.617T>C (p.Leu206Pro), NM_001134831.2(AHI1):c.1328T>A (p.Val443Asp), NM_024740.2(ALG9):c.1173+2T>A, NM_021926.4(ALX4):c.503del (p.Pro168fs), NM_001278512.2(AP3B2):c.1837del (p.Glu613fs), NM_001278512.2(AP3B2):c.2579_2582del (p.Leu860fs), NM_000039.2(APOA1):c.148G>C (p.Gly50Arg), NM_000039.2(APOA1):c.595G>C (p.Ala199Pro), NM_000039.2(APOA1):c.251T>G (p.Leu84Arg), NM_000384.3(APOB):c.10740C>G (p.Asn3580Lys), NM_000384.3(APOB):c.13168G>C (p.Asp4390His), NM_000384.3(APOB):c.11789-2A>C, NM_000384.3(APOB):c.13196A>C (p.Lys4399Thr), NM_000384.3(APOB):c.13129_13130del (p.IIe4377fs), NM_000384.3(APOB):c.3012del (p.Glu1004fs), NM_000384.3(APOB):c.39del (p.Leu14fs), NM_000384.3(APOB):c.4590del (p.Asn1531fs), NM_000384.3(APOB):c.2297_2298del (p.Lys766fs), NM_000384.3(APOB):c.819-2A>G, NM_000384.3(APOB):c.10633G>T (p.Glu3545Ter), NM_000384.3(APOB):c.5238T>G (p.Tyr1746Ter), NM_000384.3(APOB):c.905-1_905dup, NM_000384.3(APOB):c.9200del (p.Lys3067fs), NM_000384.2(APOB):c.11905del (p.Glu3969Asnfs), NM_000384.3(APOB):c.12181del (p.Glu4061fs), NM_000384.3(APOB):c.2786del (p.Pro929fs), NM_000041.4(APOE):c.490A>C (p.Lys164Gln), NM_006420.3(ARFGEF2):c.656dup (p.Val220fs), NM_139058.3(ARX):c.998C>G (p.Thr333Ser), NM_139058.3(ARX):c.34G>T (p.Glu12Ter), NM_139058.3(ARX):c.1604T>A (p.Leu535Gln), NM_139058.3(ARX):c.81C>G (p.Tyr27Ter), NM_177924.5(ASAH1):c.917+4A>G, NM_177924.5(ASAH1):c.703G>C (p.Gly235Arg), NM_177924.5(ASAH1):c.544C>G (p.Leu182Val), NM_177924.5(ASAH1):c.413A>T (p.Glu138Val), NM_001142459.2(ASB10):c.564C>A (p.Cys188Ter), NM_001690.4(ATP6V1A):c.1112A>G (p.Asp371Gly), NM_001690.4(ATP6V1A):c.298G>T (p.Asp100Tyr), NM_003921.5(BCL10):c.398dup (p.Ser134fs), NM_003921.5(BCL10):c.231dup (p.Gly78fs), NM_182641.4(BPTF):c.2366del (p.Asn789fs), NM_182641.4(BPTF):c.989del (p.Leu330fs), NM_182641.4(BPTF):c.2982_2992+1del, NM_001127221.1(CACNA1A):c.4046G>A (p.Arg1349Gln), NM_001127221.1(CACNA1A):c.2758G>T (p.Glu920Ter), NM_001127221.1(CACNA1A):c.5118T>G (p.Tyr1706Ter), NM_001127221.1(CACNA1A):c.2042del (p.Gln681fs), NM_001127221.1(CACNA1A):c.1997C>T (p.Thr666Met), NM_001127221.1(CACNA1A):c.1442del (p.Arg481fs), NM_001127221.1(CACNA1A):c.4469T>C (p.Phe1490Ser), NM_001127221.1(CACNA1A):c.4208T>G (p.Phe1403Cys), NM_001127221.1(CACNA1A):c.3846C>G (p.Tyr1282Ter), NM_001127221.1(CACNA1A):c.3797del (p.Pro1266fs), NM_001127221.1(CACNA1A):c.3414del (p.Lys1139fs), NM_001127221.1(CACNA1A):c.5126T>C (p.IIe1709Thr), NM_001127221.1(CACNA1A):c.5428A>C (p.IIe1810Leu), NM_001127221.1(CACNA1A):c.2141T>C (p.Val714Ala), NM_001127221.1(CACNA1A):c.4366G>T (p.Val1456Leu), NM_001127221.1(CACNA1A):c.1748G>A (p.Arg583Gln), NM_001127221.1(CACNA1A):c.4037G>A (p.Arg1346Gln), NM_001205293.3(CACNA1E):c.2101A>G (p.IIe701Val), NM_001159773.2(CANT1):c.511A>T (p.IIe171 Phe), NM_000388.4(CASR):c.1512_1515del (p.Phe505fs), NM_000388.4(CASR):c.164C>T (p.Pro55Leu), NM_001323289.2(CDKL5):c.1449_1452dup (p.Lys485fs), NM_020549.4(CHAT):c.669del (p.Gn223fs), NM_001271.3(CHD2):c.3787dupG (p.Val1263Glyfs), NM_001271.4(CHD2):c.628G>T (p.Glu210Ter), NM_001271.4(CHD2):c.879_883del (p.Ser293fs), NM_001271.4(CHD2):c.1808del (p.Lys603fs), NM_001271.4(CHD2):c.1452dup (p.Arg485fs), NM_001271.4(CHD2):c.4233_4236del (p.Glu1412fs), NM_001271.4(CHD2):c.4724del (p.Gly1575fs), NM_001271.4(CHD2):c.4931_4932del (p.Arg1644fs), NM_001271.4(CHD2):c.1552del (p.Gn518fs), NM_001271.4(CHD2):c.4971G>A (p.Trp1657Ter), NM_001271.4(CHD2):c.5035C>T (p.Arg1679Ter), NM_001271.4(CHD2):c.1396C>T (p.Arg466Ter), NM_000742.4(CHRNA2):c.836T>A (p.IIe279Asn), NM_000742.4(CHRNA2):c.889A>T (p.IIe297Phe), NM_000748.3(CHRNB2):c.1010T>G (p.Val337Gly), NM_000748.3(CHRNB2):c.901C>G (p.Leu301Val), NM_000748.3(CHRNB2):c.936C>G (p.IIe312Met), NM_152515.5(CKAP2L):c.751del (p.Ser251fs), NM_001854.4(COL11A1):c.3943G>T (p.Gly1315Ter), NM_001854.4(COL11A1):c.1786dup (p.Ala596fs), NM_001844.5(COL2A1):c.2710C>T (p.Arg904Cys), NM_000094.3(COL7A1):c.6900+4A>G, NM_023073.3(CPLANE1):c.7817T>A (p.Leu2606Ter), NM_005213.4(CSTA):c.64A>T (p.Lys22Ter), NM_005213.4(CSTA):c.67-2A>T, NM_000308.3(CTSA):c.1411A>G (p.Lys471Glu), NM_000308.3(CTSA):c.1372T>G (p.Phe458Val), NM_000308.3(CTSA):c.1271T>C (p.Met424Thr), NM_000308.3(CTSA):c.831+9C>G, NM_000308.3(CTSA):c.571_572del (p.Phe191fs), NM_000104.3(CYP1B1):c.1345del (p.Asp449fs), NM_000104.3(CYP1B1):c.1267A>T (p.Asn423Tyr), NM_183075.3(CYP2U1):c.947A>T (p.Asp316Val), NM_001005336.3(DNM1):c.618G>C (p.Lys206Asn), NM_001005336.3(DNM1):c.865A>T (p.IIe289Phe), NM_001005336.3(DNM1):c.709C>T (p.Arg237Trp), NM_001367561.1(DOCK7):c.4783del (p.Met1595fs), NM_001367561.1(DOCK7):c.6265G>T (p.Glu2089Ter), NM_001367561.1(DOCK7):c.2510del (p.Asp837fs), NM_173660.5(DOK7):c.331+1 G>T, NM_001942.4(DSG1):c.1861del (p.Ala621fs), NM_001958.4(EEF1A2):c.208G>A (p.Gly70Ser), NM_001013703.4(EIF2AK4):c.1153dup (p.Val385fs), NM_001013703.4(EIF2AK4):c.567dup (p.Lys190fs), NM_000799.4(EPO):c.33del (p.Trp11fs), NM_000799.4(EPO):c.20del (p.Pro7fs), NM_000400.3(ERCC2):c.2150C>G (p.Ala717Gly), NM_005236.2(ERCC4):c.1484_1488del (p.Thr495fs), NM_000125.3(ESR1):c.1125G>T (p.Gln375His), NM_014297.5(ETHE1):c.488G>A (p.Arg163Gln), NM_014297.5(ETHE1):c.554T>G (p.Leu185Arg), NM_014297.5(ETHE1):c.487C>T (p.Arg163Trp), NM_014297.5(ETHE1):c.406A>G (p.Thr136Ala), NM_014297.5(ETHE1):c.505+1G>C, NM_014297.5(ETHE1):c.230del (p.Asn77fs), NM_014297.5(ETHE1):c.440_450del (p.His147fs), NM_014297.5(ETHE1):c.66del (p.IIe23fs), NM_014297.4(ETHE1):c.−67-16_-67-12delCGCCC, NM_014297.5(ETHE1):c.3G>T (p.Met1IIe), NM_000504.4(F10):c.859A>T (p.Arg287Trp), NM_001994.2(F13B):c.1349G>T (p.Cys450Phe), NM_000130.4(F5):c.5189A>G (p.Tyr1730Cys), NM_000130.4(F5):c.1000A>G (p.Arg334Gly), NM_000131.4(F7):c.479A>G (p.Gln160Arg), NM_000131.4(F7):c.1165T>G (p.Cys389Gly), NM_000131.4(F7):c.1224T>G (p.His408Gln), NM_000132.3(F8):c.1172G>A (p.Arg391His), NM_000135.4(FANCA):c.1981A>T (p.Arg661Ter), NM_000135.4(FANCA):c.1074_1075del (p.Tyr359fs), NM_000135.4(FANCA):c.97del (p.Glu33fs), NM_000135.4(FANCA):c.2546del (p.Ser849fs), NM_000135.4(FANCA):c.3720_3724del (p.Glu1240fs), NM_000135.4(FANCA):c.1359+1G>C, NM_000135.4(FANCA):c.100A>T (p.Lys34Ter), NM_000135.4(FANCA):c.2601+1G>T, NM_000135.4(FANCA):c.1615del (p.Asp539fs), NM_000135.4(FANCA):c.1606del (p.Ser536fs), NM_000135.4(FANCA):c.4261-2A>C, NM_000135.4(FANCA):c.1827-1G>A, NM_000135.4(FANCA):c.3558dup (p.Arg1187fs), NM_000135.4(FANCA):c.862G>T (p.Glu288Ter), NM_001018113.3(FANCB):c.2150T>G (p.Leu717Ter), NM_001018113.3(FANCB):c.1668del (p.Asp557fs), NM_033084.5(FANCD2):c.1201del (p.Arg401fs), NM_004629.1(FANCG):c.156dup (p.Leu53fs), NM_004629.1(FANCG):c.925-2A>G, NM_004629.1(FANCG):c.307+1G>C, NM_004629.1(FANCG):c.637_643del (p.Tyr213fs), NM_004629.1(FANCG):c.1183_1192del (p.Glu395fs), NM_004629.1(FANCG):c.1795_1804del (p.Trp599Profs), NM_001113378.1(FANCI):c.1461T>A (p.Tyr487Ter), NM_001113378.1(FANCI):c.2422A>T (p.Lys808Ter), NM_018062.3(FANCL):c.430del (p.Ser144fs), NM_024622.6(FASTKD1):c.2230T>A (p.Tyr744Asn), NM_000507.4(FBP1):c.851C>G (p.Pro284Arg), NM_015665.6(AAAS):c.980dup (p.Ser328fs), NM_015665.6(AAAS):c.43C>A (p.Gln15Lys), NM_015665.6(AAAS):c.1432C>T (p.Arg478Ter), NM_005763.4(AASS):c.1925C>G (p.Ser642Ter), NM_005763.4(AASS):c.1256T>G (p.Leu419Arg), NM_005763.4(AASS):c.976_977del (p.Gn326fs), NM_000020.2(ACVRL1):c.150G>T (p.Trp50Cys), NM_000020.2(ACVRL1):c.1232G>C (p.Arg411 Pro), NM_001077401.2(ACVRL1):c.525+1del, NM_000020.2(ACVRL1):c.573del (p.Phe192fs), NM_001077401.2(ACVRL1):c.625+1del, NM_000020.2(ACVRL1):c.641del (p.Gly214fs), NM_000020.2(ACVRL1):c.889del (p.His297fs), NM_000020.2(ACVRL1):c.190del (p.Gn64fs), NM_000020.2(ACVRL1):c.1221G>T (p.Glu407Asp), NM_000020.2(ACVRL1):c.206G>T (p.Cys69Phe), NM_000020.2(ACVRL1):c.1147G>T (p.Glu383Ter), NM_000020.2(ACVRL1):c.1127T>G (p.Met376Arg), NM_000020.2(ACVRL1):c.1193T>A (p.IIe398Asn), NM_000020.2(ACVRL1):c.41dup (p.Met15fs), NM_000020.2(ACVRL1):c.542_545del (p.Asp181fs), NM_000020.2(ACVRL1):c.183del (p.Arg61fs), NM_000020.2(ACVRL1):c.200G>A (p.Arg67Gln), NM_000020.2(ACVRL1):c.924C>A (p.Cys308Ter), NM_000020.2(ACVRL1):c.998G>T (p.Ser333IIe), NM_000020.2(ACVRL1):c.601C>T (p.Gln201Ter), NM_000020.2(ACVRL1):c.145dup (p.Ala49fs), NM_000020.2(ACVRL1):c.406_409del (p.Gly136fs), NM_000020.2(ACVRL1):c.841G>T (p.Glu281Ter), NM_000020.2(ACVRL1):c.1435C>T (p.Arg479Ter), NM_000020.2(ACVRL1):c.430C>T (p.Arg144Ter), NM_000020.2(ACVRL1):c.1436G>A (p.Arg479Gln), NM_000020.2(ACVRL1):c.1120C>T (p.Arg374Trp), NM_000020.2(ACVRL1):c.1031G>A (p.Cys344Tyr), NM_000020.2(ACVRL1):c.1121G>A (p.Arg374Gln), NM_000020.2(ACVRL1):c.1231C>T (p.Arg411Trp), NM_000020.2(ACVRL1):c.760_762del (p.Asp254del), NM_001077401.2(ACVRL1):c.1232G>A (p.Arg411Gln), NM_000020.2(ACVRL1):c.1450C>T (p.Arg484Trp), NM_014243.3(ADAMTS3):c.503T>C (p.Leu168Pro), NM_014243.3(ADAMTS3):c.872T>C (p.IIe291Thr), NM_006721.4(ADK):c.953C>A (p.Ala318Glu), NM_001282531.3(ADNP):c.1211C>A (p.Ser404Ter), NM_001282531.3(ADNP):c.539_542del (p.Val180fs), NM_000642.3(AGL):c.4456del (p.Ser1486fs), NM_000642.3(AGL):c.3965del (p.Val1322fs), NM_000028.2(AGL):c.1999del (p.Gn667fs), NM_000642.3(AGL):c.2039G>A (p.Trp680Ter), NM_000642.3(AGL):c.853C>T (p.Arg285Ter), NM_000642.3(AGL):c.1589C>G (p.Ser530Ter), NM_000642.3(AGL):c.276del (p.Gln92fs), NM_000642.3(AGL):c.378T>A (p.Cys126Ter), NM_000642.3(AGL):c.4197del (p.Ala1400fs), NM 000642.3(AGL):c.2278del (p.Ser760fs), NM_000642.3(AGL):c.442del (p.Arg148fs), NM_000642.3(AGL):c.2590C>T (p.Arg864Ter), NM_000032.5(ALAS2):c.569A>T (p.Asp190Val), NM_000032.5(ALAS2):c.1570C>G (p.His524Asp), NM_000032.5(ALAS2):c.1163C>G (p.Thr388Ser), NM_000032.5(ALAS2):c.895A>C (p.Lys299Gln), NM_000035.4(ALDOB):c.522C>G (p.Tyr174Ter), NM_000035.4(ALDOB):c.548_553del (p.Leu183_Val184del), NM_000035.4(ALDOB):c.113-1_115del, NM_000035.4(ALDOB):c.420del (p.Asp141fs), NM_000035.4(ALDOB):c.546del (p.Leu183fs), NM_000035.4(ALDOB):c.448G>C (p.Ala150Pro), NM_213599.2(ANO5):c.989dup (p.Leu330fs), NM_213599.2(ANO5):c.1898+1G>A, NM_213599.2(ANO5):c.2272C>T (p.Arg758Cys), NM_213599.2(ANO5):c.1295C>G (p.Ala432Gly), NM_213599.2(ANO5):c.41-1G>A, NM_213599.2(ANO5):c.1627dup (p.Met543fs), NM_213599.2(ANO5):c.1520del (p.Phe507fs), NM_213599.2(ANO5):c.2004del (p.Leu669fs), NM_213599.2(ANO5):c.299del (p.Arg100fs), NM_213599.2(ANO5):c.1066T>C (p.Cys356Arg), NM_213599.2(ANO5):c.1066T>G (p.Cys356Gly), NM_058172.6(ANTXR2):c.1073dup (p.Ala359fs), NM_058172.6(ANTXR2):c.986T>G (p.Leu329Arg), NM_058172.6(ANTXR2):c.1305del (p.Thr436fs), NM_004069.5(AP2S1):c.43C>T (p.Arg15Cys), NM_003664.4(AP3B1):c.1739T>G (p.Leu580Arg), NM_003664.4(AP3B1):c.1975G>T (p.Glu659Ter), NM_003664.4(AP3B1):c.2702C>G (p.Ser901Cys), NM_003664.4(AP3B1):c.177del (p.Lys59fs), NM_000384.3(APOB):c.409G>T (p.Glu137Ter), NM_000384.3(APOB):c.11812_11813del (p.Asp3938fs), NM_000384.3(APOB):c.5116dup (p.Thr1706fs), NM_000384.3(APOB):c.3600T>A (p.Tyr1200Ter), NM_000384.3(APOB):c.2988_2994del (p.Gly997fs), NM_015915.4(ATL1):c.715C>T (p.Arg239Cys), NM_004656.4(BAP1):c.956C>G (p.Ser319Ter), NM_004656.4(BAP1):c.1882_1885del (p.Ser628fs), NM_004656.4(BAP1):c.1381del (p.IIe461fs), NM_004656.4(BAP1):c.458_459del (p.Pro153fs), NM_004656.4(BAP1):c.65del (p.Phe22fs), NM_000057.4(BLM):c.1500del (p.Phe500fs), NM_000057.4(BLM):c.1385del (p.Ser462fs), NM_212550.4(BLOC1S3):c.448del (p.Gln150fs), NM_001218.5(CA12):c.363C>A (p.His121Gln), NM_000388.4(CASR):c.2180T>A (p.Leu727Gln), NM_000388.4(CASR):c.1846C>G (p.Leu616Val), NM_000388.4(CASR):c.2318T>G (p.Leu773Arg), NM_000388.4(CASR):c.2363T>G (p.Phe788Cys), NM_000388.4(CASR):c.393C>G (p.Cys131Trp), NM_000388.4(CASR):c.374T>C (p.Leu125Pro), NM_000388.4(CASR):c.554G>A (p.Arg185Gln), NM_000388.4(CASR):c.658C>T (p.Arg220Trp), NM_000388.4(CASR):c.514A>G (p.Arg172Gly), NM_000388.4(CASR):c.186-1G>T, NM_000388.4(CASR):c.115C>G (p.Pro39Ala), NM_000071.2(CBS):c.467del (p.Leu156fs), NM_000071.2(CBS):c.19dup (p.Gln7fs), NM_000071.2(CBS):c.816T>A (p.Cys272Ter), NM_000071.2(CBS):c.1150A>G (p.Lys384Glu), NM_000071.2(CBS):c.572C>T (p.Thr191Met), NM_000071.2(CBS):c.1224-2A>C, NM_133459.4(CCBE1):c.305G>C (p.Cys102Ser), NM_133459.4(CCBE1):c.520T>C (p.Cys174Arg), NM_133459.4(CCBE1):c.979G>C (p.Gly327Arg), NM_016952.4(CDON):c.2339T>A (p.Val780Glu), NM_016952.4(CDON):c.2818A>C (p.Ser940Arg), NM_014246.3(CELSR1):c.5226+2T>A, NM_018131.5(CEP55):c.1274C>A (p.Ser425Ter), NM_032545.3(CFC1):c.522del (p.Ala175fs), NM_004366.6(CLCN2):c.1084A>T (p.Lys362Ter), NM_004366.6(CLCN2):c.76T>A (p.Tyr26Asn), NM_004366.6(CLCN2):c.65T>A (p.Met22Lys), NM_006580.3(CLDN16):c.908C>G (p.Thr303Arg), NM_014141.6(CNTNAP2):c.3408C>A (p.Tyr1136Ter), NM_001040431.3(COA3):c.199dup (p.Leu67fs), NM_198076.6(COX20):c.154A>C (p.Thr52Pro), NM_005211.3(CSF1R):c.2442+1G>T, NM_005211.3(CSF1R):c.2060dup (p.Ser688fs), NM_005211.3(CSF1R):c.2624T>C (p.Met875Thr), NM_005211.3(CSF1R):c.2381T>C (p.IIe794Thr), NM_005211.3(CSF1R):c.1754-2A>G, NM_005211.3(CSF1R):c.2378A>C (p.Lys793Thr), NM_005211.3(CSF1R):c.1889T>G (p.Leu630Arg), NM_005211.3(CSF1R):c.2528T>A (p.IIe843Asn), NM_005211.3(CSF1R):c.2527A>T (p.IIe843Phe), NM_005211.3(CSF1R):c.2562T>A (p.Asn854Lys), NM_000099.4(CST3):c.281T>A (p.Leu94Gln), NM_007272.3(CTRC):c.308del (p.Gly103fs), NM_001814.6(CTSC):c.857A>G (p.Gn286Arg), NM_000101.4(CYBA):c.246del (p.Phe83fs), NM_000397.3(CYBB):c.907C>A (p.His303Asn), NM_000397.3(CYBB):c.1244C>A (p.Pro415His), NM_000397.3(CYBB):c.1166G>C (p.Gly389Ala), NM_001033855.3(DCLRE1C):c.241C>T (p.Arg81Ter), NM_178152.3(DCX):c.532C>G (p.Arg178Gly), NM_178152.3(DCX):c.310A>T (p.IIe104Phe), NM_178152.3(DCX):c.266G>C (p.Arg89Pro), NM_178152.3(DCX):c.586C>G (p.Arg196Gly), NM_178152.3(DCX):c.766A>T (p.Lys256Ter), NM_178152.3(DCX):c.853A>T (p.Lys285Ter), NM_178152.3(DCX):c.533G>T (p.Arg178Leu), NM_178152.3(DCX):c.520A>G (p.Lys174Glu), NM_178152.3(DCX):c.1042-2A>G, NM_178152.3(DCX):c.628G>T (p.Val210Phe), NM_178152.3(DCX):c.611C>A (p.Ala204Asp), NM_178152.3(DCX):c.751G>T (p.Ala251Ser), NM_178152.3(DCX):c.409G>T (p.Glu137Ter), NM_178152.3(DCX):c.299G>T (p.Gly100Val), NM_178152.3(DCX):c.290T>G (p.Leu97Arg), NM_178152.3(DCX):c.210C>A (p.Tyr70Ter), NM_178152.3(DCX):c.150G>T (p.Lys50Asn), NM_178152.3(DCX):c.151_154del (p.Lys51fs), NM_178152.3(DCX):c.124del (p.Thr42fs), NM_178151.2(DCX):c.367del, NM_178152.3(DCX):c.478_479del (p.Gln160fs), NM_178152.3(DCX):c.494_504del (p.Ser165fs), NM_178152.3(DCX):c.578_585del (p.Lys193fs), NM_178152.3(DCX):c.574C>T (p.Arg192Trp), NM_178152.3(DCX):c.572C>G (p.Pro191Arg), NM_021800.3(DNAJC12):c.158-2A>T, NM_001130823.3(DNMT1):c.1531T>C (p.Tyr511His), NM_001122659.3(EDNRB):c.828G>T (p.Trp276Cys), NM_004247.4(EFTUD2):c.1149+1G>C, NM_004247.4(EFTUD2):c.1910T>G (p.Leu637Arg), NM_003742.4(ABCB11):c.3213+1del, NM_001098.3(ACO2):c.2105_2106del (p.Gn702fs), NM_001098.3(ACO2):c.2208G>C (p.Lys736Asn), NM_001098.3(ACO2):c.336C>G (p.Ser112Arg), NM_001098.3(ACO2):c.2338_2339del (p.Gn780fs), NM_001100.3(ACTA1):c.868G>C (p.Asp290His), NM_001101.5(ACTB):c.537C>G (p.Asp179Glu), NM_001101.5(ACTB):c.259C>G (p.His87Asp), NM_001615.4(ACTG2):c.533G>A (p.Arg178His), NM_001615.4(ACTG2):c.533G>T (p.Arg178Leu), NM_016188.5(ACTL6B):c.230A>G (p.Asp77Gly), NM_001282225.2(ADA2):c.1078A>G (p.Thr360Ala), NM_003183.6(ADAM17):c.603_606del (p.Asp201fs), NM_183357.2(ADCY5):c.1425C>G (p.IIe475Met), NM_001282531.3(ADNP):c.2287del (p.Ser763fs), NM_001282531.3(ADNP):c.2213C>G (p.Ser738Ter), NM_001134831.2(AHI1):c.1052G>T (p.Arg351Leu), NM_001134831.2(AH11):c.1115A>G (p.Asp372Gly), NM_001134831.2(AH11):c.1152-2A>G, NM_001134831.2(AH11):c.1976A>T (p.Asp659Val), NM_001134831.2(AH11):c.2705T>A (p.Val902Asp), NM_001134831.2(AH11):c.1917T>A (p.Tyr639Ter), NM_001134831.2(AH11):c.662C>G (p.Ser221Ter), NM_001134831.2(AH11):c.1897_1898dup (p.Tyr634fs), NM_001134831.2(AH11):c.1614del (p.Val539fs), NM_001134831.2(AH11):c.2172del (p.Trp725fs), NM_001134831.2(AH11):c.1267C>T (p.Gln423Ter), NM_020661.4(AICDA):c.177_185del (p.Leu59_Leu62delinsPhe), NM_006303.4(AIMP2):c.575-2A>G, NM_014336.5(AIPL1):c.715T>C (p.Cys239Arg), NM_014336.5(AIPL1):c.589G>C (p.Ala197Pro), NM_014336.5(AIPL1):c.617T>A (p.IIe206Asn), NM_001080.3(ALDH5A1):c.819del (p.Asp274fs), NM_000478.6(ALPL):c.648+1G>A, NM_000478.6(ALPL):c.1559del (p.Leu520fs), NM_000478.6(ALPL):c.18del (p.Val7fs), NM_000478.6(ALPL):c.997+2T>G, NM_000478.6(ALPL):c.129del (p.Gln44fs), NM_000478.6(ALPL):c.114del (p.Lys38fs), NM_000478.6(ALPL):c.620A>C (p.Gn207Pro), NM_000478.6(ALPL):c.46_49del (p.Asn16fs), NM_020919.4(ALS2):c.1007_1008del (p.IIe336fs), NM_020919.4(ALS2):c.4721del (p.Val1574fs), NM_020919.3(ALS2):c.1472_1481delTTTCCCCCAG, NM_020919.4(ALS2):c.3520A>T (p.Lys1174Ter), NM_020919.4(ALS2):c.1425_1428del (p.Gly477fs), NM_020919.4(ALS2):c.1867_1868del (p.Leu623fs), NM 020919.4(ALS2):c.2980-2A>G, NM_152424.4(AMER1):c.1072C>T (p.Arg358Ter), NM_013275.6(ANKRD11):c.7303del (p.Ala2435fs), NM_013275.6(ANKRD11):c.4902del (p.Leu1635fs), NM_013275.6(ANKRD11):c.4114G>T (p.Glu1372Ter), NM_013275.6(ANKRD11):c.1389del (p.Gly464fs), NM_013275.6(ANKRD11):c.1623_1630del (p.His542fs), NM_013275.6(ANKRD11):c.5537del (p.Leu1846fs), NM_013275.6(ANKRD11):c.6847C>T (p.Gn2283Ter), NM_013275.6(ANKRD11):c.6212C>G (p.Ser2071Ter), NM_013275.6(ANKRD11):c.3198_3199del (p.His1066fs), NM_013275.6(ANKRD11):c.1679C>G (p.Ser560Ter), NM_013275.6(ANKRD11):c.6792del (p.Ala2265fs), NM_013275.6(ANKRD11):c.3632_3633del (p.Lys1211fs), NM_013275.6(ANKRD11):c.6472G>T (p.Glu2158Ter), NM_013275.6(ANKRD11):c.6015dup (p.Gly2006fs), NM_013275.6(ANKRD11):c.6210_6211del (p.Lys2070fs), NM_000216.4(ANOS1):c.1A>T (p.Met1Leu), NM_000216.4(ANOS1):c.1449+2del, NM_001253852.3(AP4B1):c.1345A>T (p.Arg449Ter), NM_001253852.3(AP4B1):c.405_409del (p.Tyr135_Arg137delinsTer), NM_153000.5(APCDD1):c.26T>G (p.Leu9Arg), NM_000044.6(AR):c.2610T>G (p.IIe870Met), NM_014783.6(ARHGAP11A):c.2370_2371del (p.Thr790_Cys791insTer), NM_020732.3(ARID1B):c.3183C>G (p.Tyr1061Ter), NM_020732.3(ARID1B):c.3993T>A (p.Tyr1331Ter), NM_020732.3(ARID1B):c.4171_4172del (p.Met1391fs), NM_001174150.2(ARL13B):c.461A>G (p.Asn154Ser), NM_001290020.2(ARMC4):c.1669G>T (p.Glu557Ter), NM_139058.3(ARX):c.922G>T (p.Glu308Ter), NM_177924.5(ASAH1):c.850G>T (p.Gly284Ter), NM_018136.5(ASPM):c.10168C>T (p.Arg3390Ter), NM_030632.3(ASXL3):c.1354del (p.Glu452fs), NM_000701.8(ATP1A1):c.2576T>G (p.Met859Arg), NM_001183.6(ATP6AP1):c.542T>G (p.Leu181Arg), NM_020632.3(ATP6VOA4):c.777del (p.Met259fs), NM_001015878.2(AURKC):c.145del (p.Leu49fs), NM_015570.4(AUTS2):c.856A>T (p.Lys286Ter), NM_000054.6(AVPR2):c.382_384del (p.Tyr128del), NM_001478.5(B4GALNT1):c.263dup (p.Leu89fs), NM_022893.4(BCL11A):c.385+2T>C, NM_022893.4(BCL11A):c.139A>C (p.Thr47Pro), NM_004333.6(BRAF):c.1574T>A (p.Leu525Gln), NM_004333.6(BRAF):c.722C>G (p.Thr241Arg), NM_152743.4(BRAT1):c.803+1G>C, NM_152743.4(BRAT1):c.638dup (p.Val214fs), NM_001003694.2(BRPF1):c.2308_2309del (p.Asp770fs), NM_001003694.2(BRPF1):c.28_29del (p.Phe10fs), NM_001003694.2(BRPF1):c.567del (p.Asp190fs), NM_001003694.2(BRPF1):c.2982C>G (p.Tyr994Ter), NM_001735.2(C5):c.1115A>G (p.Lys372Arg), NM_000719.7(CACNA1C):c.3497T>C (p.IIe1166Thr), NM_000069.3(CACNA1S):c.2627T>A (p.Val876Glu), NM_000069.3(CACNA1S):c.3716G>A (p.Arg1239His), NM_000069.3(CACNA1S):c.1583G>A (p.Arg528His), NM_000069.3(CACNA1S):c.3715C>G (p.Arg1239Gly), NM_015981.4(CAMK2A):c.548A>T (p.Glu183Val), NM_015981.4(CAMK2A):c.856A>C (p.Thr286Pro), NM_015981.4(CAMK2A):c.327G>C (p.Glu109Asp), NM_015215.4(CAMTA1):c.420del (p.Lys141fs), NM_001013838.3(CARMIL2):c.1574T>A (p.Leu525Gln), NM_001013838.3(CARMIL2):c.1916T>A (p.Leu639His), NM_003688.3(CASK):c.2065A>T (p.Lys689Ter), NM_012114.3(CASP14):c.462_463del (p.Asp154fs), NM_001080522.2(CC2D2A):c.3744_3747dup (p.Pro1250fs), NM_001080522.2(CC2D2A):c.3134T>C (p.Val1045Ala), NM_001080522.2(CC2D2A):c.2999A>T (p.Glu1000Val), NM_001080522.2(CC2D2A):c.2624C>A (p.Ser875Ter), NM_001080522.2(CC2D2A):c.1676T>C (p.Leu559Pro), NM_001080522.2(CC2D2A):c.4289T>C (p.Val1430Ala), NM_001080522.2(CC2D2A):c.3892_3893del (p.Val1298fs), NM_001080522.2(CC2D2A):c.2848C>T (p.Arg950Ter), NM_001080522.2(CC2D2A):c.1017+1G>A, NM_198053.2(CD247):c.209A>T (p.Gln70Leu), NM_000733.3(CD3E):c.128_129del (p.Thr43fs), NM_001250.6(CD40):c.257-2A>T, NM_001250.6(CD40):c.247T>C (p.Cys83Arg), NM_000074.2(CD40LG):c.368C>A (p.Ala123Glu), NM_000074.2(CD40LG):c.31C>T (p.Arg11Ter), NM_000074.2(CD40LG):c.559del (p.Ala187fs), NM_001791.4(CDC42):c.196A>G (p.Arg66Gly), NM_031942.5(CDCA7):c.1118del (p.Gly373fs), NM_022124.6(CDH23):c.1428dup (p.Thr477fs), NM_001793.6(CDH3):c.981del (p.Met327fs), NM_001323289.2(CDKL5):c.1107_1120del (p.Gly369_Asn370insTer), NM_000076.2(CDKN1C):c.832A>G (p.Lys278Glu), NM_000076.2(CDKN1C):c.827T>C (p.Phe276Ser), NM_000076.2(CDKN1C):c.826T>G (p.Phe276Val), NM_000076.2(CDKN1C):c.815T>G (p.IIe272Ser), NM_014704.4(CEP104):c.759T>G (p.Tyr253Ter), NM_025114.3(CEP290):c.103-1G>T, NM_025114.3(CEP290):c.3185delT (p.Leu1062Argfs), NM_025114.3(CEP290):c.4522C>T (p.Arg1508Ter), NM_025114.3(CEP290):c.1984C>T (p.Gln662Ter), NM_025114.3(CEP290):c.2112delA (p.Val705Leufs), NM_025114.3(CEP290):c.1451delA (p.Lys484Argfs), NM_025114.3(CEP290):c.5931_5935delACGAG (p.Arg1978Phefs), NM_025114.3:c.4028delA, NM_025114.3(CEP290):c.1910-11T>G, NM_001042384.2(CEP63):c.448del (p.Arg150fs), NM_032436.4(CHAMP1):c.1489C>T (p.Arg497Ter), NM_001005273.2(CHD3):c.2745G>T (p.Leu915Phe), NM_001005273.2(CHD3):c.3472T>C (p.Trp1158Arg), NM_001005273.2(CHD3):c.3505C>T (p.Arg1169Trp), NM_001005273.2(CHD3):c.3515G>A (p.Arg1172Gln), NM_001005273.2(CHD3):c.3362G>C (p.Arg1121 Pro), NM_017780.4(CHD7):c.5167dup (p.Gln1723fs), NM_020920.4(CHD8):c.1994_1998del (p.Cys665fs), NM_021101.5(CLDN1):c.358del (p.Val120fs), NM_021101.5(CLDN1):c.200_201del (p.Val66_Phe67insTer), NM_148960.3(CLDN19):c.388G>T (p.Gly130Cys), NM_148960.3(CLDN19):c.269T>G (p.Leu90Arg), NM_024769.4(CLMP):c.664C>T (p.Arg222Ter), NM_024769.4(CLMP):c.371T>A (p.Val124Asp), NM_024769.4(CLMP):c.230del (p.Glu77fs), NM_000086.2(CLN3):c.1198-1G>T, NM_000086.2(CLN3):c.558_559del (p.Gly187fs), NM_000086.2(CLN3):c.424del (p.Val142fs), NM_000086.2(CLN3):c.622dup (p.Ser208fs), NM_000086.2(CLN3):c.1054C>T (p.Gn352Ter), NM_000086.2(CLN3):c.906+2T>A, NM_001854.4(COL11A1):c.3816+1G>A, NM_130445.2(COL18A1):c.2118dupC (p.Gly707Argfs), NM_030582.4(COL18A1):c.4349+2T>C, NM_030582.4(COL18A1):c.3509_3518del (p.Pro1170fs), NM_030582.4(COL18A1):c.4054_4055del (p.Leu1352fs), NM_003742.4(ABCB11):c.3213+1del, NM_001098.3(ACO2):c.2105_2106del (p.Gn702fs), NM_001098.3(ACO2):c.2208G>C (p.Lys736Asn), NM_001098.3(ACO2):c.336C>G (p.Ser112Arg), NM_001098.3(ACO2):c.2338_2339del (p.Gn780fs), NM_001100.3(ACTA1):c.868G>C (p.Asp290His), NM_001101.5(ACTB):c.537C>G (p.Asp179Glu), NM_001101.5(ACTB):c.259C>G (p.His87Asp), NM_001615.4(ACTG2):c.533G>A (p.Arg178His), NM_001615.4(ACTG2):c.533G>T (p.Arg178Leu), NM_016188.5(ACTL6B):c.230A>G (p.Asp77Gly), NM_001282225.2(ADA2):c.1078A>G (p.Thr360Ala), NM_003183.6(ADAM17):c.603_606del (p.Asp201fs), NM_183357.2(ADCY5):c.1425C>G (p.IIe475Met), NM_001282531.3(ADNP):c.2287del (p.Ser763fs), NM_001282531.3(ADNP):c.2213C>G (p.Ser738Ter), NM_001134831.2(AH11):c.1052G>T (p.Arg351Leu), NM_001134831.2(AH11):c.1115A>G (p.Asp372Gly), NM_001134831.2(AH11):c.1152-2A>G, NM_001134831.2(AH11):c.1976A>T (p.Asp659Val), NM_001134831.2(AH11):c.2705T>A (p.Val902Asp), NM_001134831.2(AH11):c.1917T>A (p.Tyr639Ter), NM_001134831.2(AH11):c.662C>G (p.Ser221Ter), NM_001134831.2(AH11):c.1897_1898dup (p.Tyr634fs), NM_001134831.2(AH11):c.1614del (p.Val539fs), NM_001134831.2(AH11):c.2172del (p.Trp725fs), NM_001134831.2(AH11):c.1267C>T (p.Gln423Ter), NM_020661.4(AICDA):c.177_185del (p.Leu59_Leu62delinsPhe), NM_006303.4(AIMP2):c.575-2A>G, NM_014336.5(AIPL1):c.715T>C (p.Cys239Arg), NM_014336.5(AIPL1):c.589G>C (p.Ala197Pro), NM_014336.5(AIPL1):c.617T>A (p.IIe206Asn), NM_001080.3(ALDH5A1):c.819del (p.Asp274fs), NM_000478.6(ALPL):c.648+1G>A, NM_000478.6(ALPL):c.1559del (p.Leu520fs), NM_000478.6(ALPL):c.18del (p.Val7fs), NM_000478.6(ALPL):c.997+2T>G, NM_000478.6(ALPL):c.129del (p.Gn44fs), NM_000478.6(ALPL):c.114del (p.Lys38fs), NM_000478.6(ALPL):c.620A>C (p.Gln207Pro), NM_000478.6(ALPL):c.46_49del (p.Asn16fs), NM_020919.4(ALS2):c.1007_1008del (p.IIe336fs), NM_020919.4(ALS2):c.4721del (p.Val1574fs), NM_020919.3(ALS2):c.1472_1481delTTTCCCCCAG, NM_020919.4(ALS2):c.3520A>T (p.Lys1174Ter), NM_020919.4(ALS2):c.1425_1428del (p.Gly477fs), NM_020919.4(ALS2):c.1867_1868del (p.Leu623fs), NM 020919.4(ALS2):c.2980-2A>G, NM_152424.4(AMER1):c.1072C>T (p.Arg358Ter), NM_013275.6(ANKRD11):c.7303del (p.Ala2435fs), NM_013275.6(ANKRD11):c.4902del (p.Leu1635fs), NM_013275.6(ANKRD11):c.4114G>T (p.Glu1372Ter), NM_013275.6(ANKRD11):c.1389del (p.Gly464fs), NM_013275.6(ANKRD11):c.1623_1630del (p.His542fs), NM_013275.6(ANKRD11):c.5537del (p.Leu1846fs), NM_013275.6(ANKRD11):c.6847C>T (p.Gn2283Ter), NM_013275.6(ANKRD11):c.6212C>G (p.Ser2071Ter), NM_013275.6(ANKRD11):c.3198_3199del (p.His1066fs), NM_013275.6(ANKRD11):c.1679C>G (p.Ser560Ter), NM_013275.6(ANKRD11):c.6792del (p.Ala2265fs), NM_013275.6(ANKRD11):c.3632_3633del (p.Lys1211fs), NM_013275.6(ANKRD11):c.6472G>T (p.Glu2158Ter), NM_013275.6(ANKRD11):c.6015dup (p.Gly2006fs), NM_013275.6(ANKRD11):c.6210_6211del (p.Lys2070fs), NM_000216.4(ANOS1):c.1A>T (p.Met1Leu), NM_000216.4(ANOS1):c.1449+2del, NM_001253852.3(AP4B1):c.1345A>T (p.Arg449Ter), NM_001253852.3(AP4B1):c.405_409del (p.Tyr135_Arg137delinsTer), NM_153000.5(APCDD1):c.26T>G (p.Leu9Arg), NM_000044.6(AR):c.2610T>G (p.IIe870Met), NM_014783.6(ARHGAP11A):c.2370_2371del (p.Thr790_Cys791insTer), NM_020732.3(ARID1B):c.3183C>G (p.Tyr1061Ter), NM_020732.3(ARID1B):c.3993T>A (p.Tyr1331Ter), NM_020732.3(ARID1B):c.4171_4172del (p.Met1391fs), NM_001174150.2(ARL13B):c.461A>G (p.Asn154Ser), NM_001290020.2(ARMC4):c.1669G>T (p.Glu557Ter), NM_139058.3(ARX):c.922G>T (p.Glu308Ter), NM_177924.5(ASAH1):c.850G>T (p.Gly284Ter), NM_018136.5(ASPM):c.10168C>T (p.Arg3390Ter), NM_030632.3(ASXL3):c.1354del (p.Glu452fs), NM_000701.8(ATP1A1):c.2576T>G (p.Met859Arg), NM_001183.6(ATP6AP1):c.542T>G (p.Leu181Arg), NM_020632.3(ATP6VOA4):c.777del (p.Met259fs), NM_001015878.2(AURKC):c.145del (p.Leu49fs), NM_015570.4(AUTS2):c.856A>T (p.Lys286Ter), NM_000054.6(AVPR2):c.382_384del (p.Tyr128del), NM_001478.5(B4GALNT1):c.263dup (p.Leu89fs), NM_022893.4(BCL11A):c.385+2T>C, NM_022893.4(BCL11A):c.139A>C (p.Thr47Pro), NM_004333.6(BRAF):c.1574T>A (p.Leu525Gln), NM_004333.6(BRAF):c.722C>G (p.Thr241Arg), NM_152743.4(BRAT1):c.803+1G>C, NM_152743.4(BRAT1):c.638dup (p.Val214fs), NM_001003694.2(BRPF1):c.2308_2309del (p.Asp770fs), NM_001003694.2(BRPF1):c.28_29del (p.Phe10fs), NM_001003694.2(BRPF1):c.567del (p.Asp190fs), NM_001003694.2(BRPF1):c.2982C>G (p.Tyr994Ter), NM_001735.2(C5):c.1115A>G (p.Lys372Arg), NM_000719.7(CACNA1C):c.3497T>C (p.IIe1166Thr), NM_000069.3(CACNA1S):c.2627T>A (p.Val876Glu), NM_000069.3(CACNA1S):c.3716G>A (p.Arg1239His), NM_000069.3(CACNA1S):c.1583G>A (p.Arg528His), NM_000069.3(CACNA1S):c.3715C>G (p.Arg1239Gly), NM_015981.4(CAMK2A):c.548A>T (p.Glu183Val), NM_015981.4(CAMK2A):c.856A>C (p.Thr286Pro), NM_015981.4(CAMK2A):c.327G>C (p.Glu109Asp), NM_015215.4(CAMTA1):c.420del (p.Lys141fs), NM_001013838.3(CARMIL2):c.1574T>A (p.Leu525Gln), NM_001013838.3(CARMIL2):c.1916T>A (p.Leu639His), NM_003688.3(CASK):c.2065A>T (p.Lys689Ter), NM_012114.3(CASP14):c.462_463del (p.Asp154fs), NM_001080522.2(CC2D2A):c.3744_3747dup (p.Pro1250fs), NM_001080522.2(CC2D2A):c.3134T>C (p.Val1045Ala), NM_001080522.2(CC2D2A):c.2999A>T (p.Glu1000Val), NM_001080522.2(CC2D2A):c.2624C>A (p.Ser875Ter), NM_001080522.2(CC2D2A):c.1676T>C (p.Leu559Pro), NM_001080522.2(CC2D2A):c.4289T>C (p.Val1430Ala), NM_001080522.2(CC2D2A):c.3892_3893del (p.Val298fs), NM_001080522.2(CC2D2A):c.2848C>T (p.Arg950Ter), NM_001080522.2(CC2D2A):c.1017+1G>A, NM_198053.2(CD247):c.209A>T (p.Gln70Leu), NM_000733.3(CD3E):c.128_129del (p.Thr43fs), NM_001250.6(CD40):c.257-2A>T, NM_001250.6(CD40):c.247T>C (p.Cys83Arg), NM_000074.2(CD40LG):c.368C>A (p.Ala123Glu), NM_000074.2(CD40LG):c.31C>T (p.Arg11Ter), NM_000074.2(CD40LG):c.559del (p.Ala187fs), NM_001791.4(CDC42):c.196A>G (p.Arg66Gly), NM_031942.5(CDCA7):c.1118del (p.Gly373fs), NM_022124.6(CDH23):c.1428dup (p.Thr477fs), NM_001793.6(CDH3):c.981del (p.Met327fs), NM_001323289.2(CDKL5):c.1107_1120del (p.Gly369_Asn370insTer), NM_000076.2(CDKN1C):c.832A>G (p.Lys278Glu), NM_000076.2(CDKN1C):c.827T>C (p.Phe276Ser), NM_000076.2(CDKN1C):c.826T>G (p.Phe276Val), NM_000076.2(CDKN1C):c.815T>G (p.IIe272Ser), NM_014704.4(CEP104):c.759T>G (p.Tyr253Ter), NM_025114.3(CEP290):c.103-1G>T, NM_025114.3(CEP290):c.3185delT (p.Leu1062Argfs), NM_025114.3(CEP290):c.4522C>T (p.Arg1508Ter), NM_025114.3(CEP290):c.1984C>T (p.Gln662Ter), NM_025114.3(CEP290):c.2112delA (p.Val705Leufs), NM_025114.3(CEP290):c.1451delA (p.Lys484Argfs), NM_025114.3(CEP290):c.5931_5935delACGAG (p.Arg1978Phefs), NM_025114.3:c.4028delA, NM_025114.3(CEP290):c.1910-11T>G, NM_001042384.2(CEP63):c.448del (p.Arg150fs), NM_032436.4(CHAMP1):c.1489C>T (p.Arg497Ter), NM_001005273.2(CHD3):c.2745G>T (p.Leu915Phe), NM_001005273.2(CHD3):c.3472T>C (p.Trp1158Arg), NM_001005273.2(CHD3):c.3505C>T (p.Arg1169Trp), NM_001005273.2(CHD3):c.3515G>A (p.Arg1172Gln), NM_001005273.2(CHD3):c.3362G>C (p.Arg1121 Pro), NM_017780.4(CHD7):c.5167dup (p.Gln1723fs), NM_020920.4(CHD8):c.1994_1998del (p.Cys665fs), NM_021101.5(CLDN1):c.358del (p.Val120fs), NM_021101.5(CLDN1):c.200_201del (p.Val66_Phe67insTer), NM_148960.3(CLDN19):c.388G>T (p.Gly130Cys), NM_148960.3(CLDN19):c.269T>G (p.Leu90Arg), NM_024769.4(CLMP):c.664C>T (p.Arg222Ter), NM_024769.4(CLMP):c.371T>A (p.Val124Asp), NM_024769.4(CLMP):c.230del (p.Glu77fs), NM_000086.2(CLN3):c.1198-1G>T, NM_000086.2(CLN3):c.558_559del (p.Gly187fs), NM_000086.2(CLN3):c.424del (p.Val142fs), NM_000086.2(CLN3):c.622dup (p.Ser208fs), NM_000086.2(CLN3):c.1054C>T (p.Gn352Ter), NM_000086.2(CLN3):c.906+2T>A, NM_001854.4(COL11A1):c.3816+1G>A, NM_130445.2(COL18A1):c.2118dupC (p.Gly707Argfs), NM_030582.4(COL18A1):c.4349+2T>C, NM_030582.4(COL18A1):c.3509_3518del (p.Pro1170fs), NM_030582.4(COL18A1):c.4054_4055del (p.Leu1352fs), NM_020745.4(AARS2):c.149T>G (p.Phe50Cys), NM_000350.3(ABCA4):c.4537del (p.Gln1513fs), NM_000350.3(ABCA4):c.4537dup (p.Gln1513fs), NM_005050.4(ABCD4):c.956A>G (p.Tyr319Cys), NM_005050.4(ABCD4):c.542+1G>T, NM_005157.6(ABL1):c.707A>T (p.Glu236Val), NM_000016.5(ACADM):c.985A>C (p.Lys329Gln), NM_000016.5(ACADM):c.1190A>C (p.Tyr397Ser), NM_000016.5(ACADM):c.946-6T>G, NM_000016.5(ACADM):c.343_348del (p.Gly115_Cys116del), NM_000016.5(ACADM):c.843A>T (p.Arg281Ser), NM_000016.5(ACADM):c.1073del (p.Lys358fs), NM_000016.5(ACADM):c.437del (p.Leu146fs), NM_000016.5(ACADM):c.431_434del (p.Lys144fs), NM_000016.5(ACADM):c.233T>C (p.IIe78Thr), NM_000016.5(ACADM):c.946-2A>C, NM_000016.5(ACADM):c.449_452del (p.Thr150fs), NM_000016.5(ACADM):c.224del (p.Val75fs), NM_138422.4(ADAT3):c.430G>A (p.Val144Met), NM_005465.7(AKT3):c.686A>G (p.Asn229Ser), NM_015365.3(AMMECR1):c.429T>A (p.Tyr143Ter), NM_030943.3(AMN):c.1014_1021del (p.Leu339fs), NM_030943.3(AMN):c.1006+34_1007-31del, NM_020987.5(ANK3):c.10995del (p.Thr3666fs), NM_213599.2(ANO5):c.1210C>T (p.Arg404Ter), NM_006015.6(ARID1A):c.1113del (p.Gln372fs), NM_032131.6(ARMC2):c.2279T>A (p.IIe760Asn), NM_000487.6(ARSA):c.1010A>T (p.Asp337Val), NM_000487.6(ARSA):c.229G>C (p.Ala77Pro), NM_000487.6(ARSA):c.495_501del (p.Pro166fs), NM_000487.6(ARSA):c.1388del (p.Leu463fs), NM_000487.6(ARSA):c.1223_1231del (p.Ser408_Thr410del), NM_000487.6(ARSA):c.979_979+3del, NM_000487.6(ARSA):c.410T>C (p.Leu137Pro), NM_000487.6(ARSA):c.1279C>A (p.Pro427Thr), NM_000487.6(ARSA):c.905G>T (p.Cys302Phe), NM_000487.6(ARSA):c.862A>C (p.Thr288Pro), NM_000487.6(ARSA):c.465+1G>A, NM_000487.6(ARSA):c.1210+1G>A, NM_000487.6(ARSA):c.257G>A (p.Arg86Gln), NM_000487.6(ARSA):c.890C>A (p.Ser297Tyr), NM_000487.6(ARSA):c.769G>C (p.Asp257His), NM_000487.6(ARSA):c.641C>T (p.Ala214Val), NM_000487.6(ARSA):c.1108-2A>G, NM_000487.6(ARSA):c.302G>T (p.Gly101Val), NM_139058.3(ARX):c.1372del (p.Ala458fs), NM_139058.3(ARX):c.617del (p.Gly206fs), NM_139058.3(ARX):c.1028T>A (p.Leu343Gln), NM_139058.3(ARX):c.995G>T (p.Arg332Leu), NM_139058.3(ARX):c.1096del (p.Asp366fs), NM_139058.3(ARX):c.1141del (p.Ala381fs), NM_018489.3(ASH1L):c.2170G>T (p.Ala724Ser), NM_018489.3(ASH1L):c.3664_3667del (p.Lys1222fs), NM_001687.5(ATP5F1D):c.317T>G (p.Val106Gly), NM_000052.7(ATP7A):c.2498+2T>A, NM_000052.7(ATP7A):c.3920C>G (p.Pro1307Arg), NM_000052.7(ATP7A):c.4005+1G>T, NM_000052.7(ATP7A):c.2172+5G>C, NM_000052.7(ATP7A):c.1874T>G (p.Leu625Ter), NM_000052.7(ATP7A):c.3124del (p.Val1042fs), NM_000052.7(ATP7A):c.3340del (p.Val1114fs), NM_000052.7(ATP7A):c.1355del (p.Val452fs), NM_000052.7(ATP7A):c.3920del (p.Pro1307fs), NM_000052.7(ATP7A):c.3537del (p.Val1180fs), NM_000052.7(ATP7A):c.3069_3083del (p.IIe1024_Gly1028del), NM_000052.7(ATP7A):c.2916+3_2916+6del, NM_000052.7(ATP7A):c.4132dup (p.Met1378fs), NM_000052.7(ATP7A):c.2160T>A (p.Cys720Ter), NM_000052.7(ATP7A):c.1020_1024dup (p.Leu342fs), NM_000489.5(ATRX):c.536A>G (p.Asn179Ser), NM_015570.4(AUTS2):c.857_858del (p.Lys286fs), NM_030578.4(B9D2):c.301A>C (p.Ser101Arg), NM_000709.4(BCKDHA):c.470A>C (p.Gln157Pro), NM_000709.4(BCKDHA):c.143del (p.Leu48fs), NM_000709.4(BCKDHA):c.554del (p.Leu185fs), NM_000709.4(BCKDHA):c.1198A>T (p.Lys400Ter), NM_000709.4(BCKDHA):c.929C>G (p.Thr310Arg), NM_000709.4(BCKDHA):c.1312T>A (p.Tyr438Asn), NM_000709.4(BCKDHA):c.861_868del (p.Gly288fs), NM_000709.4(BCKDHA):c.117del (p.Arg40fs), NM_000709.4(BCKDHA):c.661_664del (p.Tyr221fs), NM_183050.4(BCKDHB):c.1065del (p.Pro356fs), NM_183050.4(BCKDHB):c.964A>G (p.Thr322Ala), NM_183050.4(BCKDHB):c.401T>A (p.IIe134Asn), NM_183050.4(BCKDHB):c.348del (p.Asp117fs), NM_183050.4(BCKDHB):c.564T>A (p.Cys188Ter), NM_183050.4(BCKDHB):c.79_89del (p.Pro27fs), NM_183050.4(BCKDHB):c.730del (p.Tyr244fs), NM_183050.4(BCKDHB):c.139_149del (p.Gn47fs), NM_183050.4(BCKDHB):c.1119del (p.Phe374fs), NM_183050.4(BCKDHB):c.356T>G (p.Val119Gly), NM_183050.4(BCKDHB):c.487G>T (p.Glu163Ter), NM_183050.4(BCKDHB):c.1022T>A (p.IIe341Asn), NM_001711.6(BGN):c.908A>C (p.Gn303Pro), NM_017637.6(BNC2):c.2663A>G (p.His888Arg), NM_153252.5(BRWD3):c.946dup (p.Arg316fs), NM_000719.7(CACNA1C):c.1552C>T (p.Arg518Cys), NM_000719.7(CACNA1C):c.1114-316G>A, NM_001743.6(CALM2):c.293A>T (p.Asn98IIe), NM_001743.6(CALM2):c.287A>T (p.Asp96Val), NM_005184.4(CALM3):c.281A>C (p.Asp94Ala), NM_001220.5(CAMK2B):c.852A>T (p.Arg284Ser), NM_000070.3(CAPN3):c.483del (p.IIe162fs), NM_000070.3(CAPN3):c.439C>T (p.Arg147Ter), NM_000070.3(CAPN3):c.1795dup (p.Thr599fs), NM_000070.3(CAPN3):c.598_612del (p.Phe200_Leu204del), NM_000070.3(CAPN3):c.2051-1G>T, NM_000070.3(CAPN3):c.1714C>T (p.Arg572Trp), NM_000070.3(CAPN3):c.1465C>T (p.Arg489Trp), NM_000070.3(CAPN3):c.223dup (p.Tyr75fs), NM_000070.3(CAPN3):c.258dup (p.Leu87fs), NM_000070.3(CAPN3):c.1992+1G>T, NM_000070.3(CAPN3):c.1027G>T (p.Glu343Ter), NM_000070.3(CAPN3):c.1699G>T (p.Gly567Trp), NM_000070.3(CAPN3):c.1662C>G (p.Tyr554Ter), NM_000070.3(CAPN3):c.580del (p.Ser194fs), NM_003688.3(CASK):c.2303-2A>G, NM_003688.3(CASK):c.1315-7A>G, NM_003688.3(CASK):c.430-2A>T, NM_003688.3(CASK):c.1981del (p.Leu661fs), NM_003688.3(CASK):c.1644_1645del (p.Val549fs), NM_003688.3(CASK):c.68del (p.Phe23fs), NM_003688.3(CASK):c.2534_2535del (p.Phe845fs), NM_033337.2(CAV3):c.40G>C (p.Val14Leu), NM_033337.2(CAV3):c.236T>G (p.Leu79Arg), NM_033337.2(CAV3):c.423C>G (p.Ser141Arg), NM_001234.5(CAV3):c.10_17del (p.Glu4fs), NM_017721.5(CC2D1A):c.748+1G>T, NM_001080522.2(CC2D2A):c.3584del (p.Phe1195fs), NM_001080522.2(CC2D2A):c.3084del (p.Lys1029fs), NM_001759.4(CCND2):c.808A>T (p.Lys270Ter), NM_001759.4(CCND2):c.842C>G (p.Pro281Arg), NM_001759.4(CCND2):c.839C>A (p.Thr280Asn), NM_025114.3(CEP290):c.4621del (p.Thr1541 Profs), NM_032436.4(CHAMP1):c.1866_1867del (p.Asp622fs), NM_032436.4(CHAMP1):c.635del (p.Pro212fs), NM_001143981.2(CHRDL1):c.782G>T (p.Cys261 Phe), NM_001143981.2(CHRDL1):c.301+2T>G, NM_001143981.2(CHRDL1):c.872del (p.Cys291fs), NM_005199.5(CHRNG):c.320T>G (p.Val107Gly), NM_021615.5(CHST6):c.521A>G (p.Lys174Arg), NM_021615.5(CHST6):c.304T>G (p.Cys102Gly), NM_021615.5(CHST6):c.599T>G (p.Leu200Arg), NM_001206999.2(CIT):c.689A>T (p.Asp230Val), NM_001206999.2(CIT):c.753+3A>T, NM_004366.6(CLCN2):c.828dup (p.Arg277fs), NM_004366.6(CLCN2):c.1304del (p.Leu435fs), NM_004366.6(CLCN2):c.1422_1423del (p.Glu475fs), NM_004366.6(CLCN2):c.1957A>T (p.Arg653Ter), NM_004366.6(CLCN2):c.1143del (p.Gly382fs), NM_001830.4(CLCN4):c.661C>G (p.Leu221Val), NM_004859.4(CLTC):c.977_980del (p.Ser326fs), NM_003632.3(CNTNAP1):c.1561dup (p.Leu521fs), NM_003632.3(CNTNAP1):c.2993-1_2995del, NM_003632.3(CNTNAP1):c.3009dup (p.Glu1004Ter), NM_003632.3(CNTNAP1):c.2901_2902del (p.Cys968fs), NM_000493.4(COL10A1):c.1884C>G (p.Tyr628Ter), NM_000493.4(COL10A1):c.1792T>G (p.Tyr598Asp), NM_001854.4(COL11A1):c.2927G>T (p.Gly976Val), NM_006438.5(COLEC10):c.228del (p.Gly77fs), NM_001866.3(COX7B):c.196del (p.Leu66fs), NM_003805.5(CRADD):c.491T>G (p.Phe164Cys), NM_201253.3(CRB1):c.1431del (p.Ser478fs), NM_017541.4(CRYGS):c.53G>T (p.Gly18Val), NM_006565.4(CTCF):c.375dup (p.Val126fs), NM_006565.4(CTCF):c.1186dup (p.Arg396fs), NM_006565.4(CTCF):c.773_776del (p.Lys258fs), NM_001904.4(CTNNB1):c.495+1G>C, NM_001904.4(CTNNB1):c.705dup (p.Gly236fs), NM_001081.3(CUBN):c.3329+1G>T, NM_001081.3(CUBN):c.5428C>T (p.Arg1810Ter), NM_001081.3(CUBN):c.6928_6934del (p.Glu2310fs), NM_001081.3(CUBN):c.7955C>A (p.Ser2652Ter), NM_001081.3(CUBN):c.3096del (p.Ala1031_Tyr1032insTer), NM_001081.3(CUBN):c.1865del (p.Thr622fs), NM_148923.4(CYB5A):c.131A>T (p.His44Leu), NM_000398.7(CYB5R3):c.610T>C (p.Cys204Arg), NM_000398.7(CYB5R3):c.382T>C (p.Ser128Pro), NM_000398.7(CYB5R3):c.817_819del (p.Met273del), NM_000350.3(ABCA4):c.1025_1038del (p.Asp342fs), NM_019112.3(ABCA7):c.4208delT (p.Leu1403Argfs), NM_003742.4(ABCB11):c.1966_1967del (p.Leu656fs), NM_001287174.2(ABCC8):c.394T>G (p.Phe132Val), NM_000018.4(ACADVL):c.889_891del (p.Glu297del), NM_000018.4(ACADVL):c.602_603del (p.Tyr201fs), NM_013227.3(ACAN):c.7265_7275del (p.His2422fs), NM_001100.3(ACTA1):c.1007A>C (p.Glu336Ala), NM_001100.3(ACTA1):c.493G>C (p.Val165Leu), NM_001100.3(ACTA1):c.591G>T (p.Glu197Asp), NM_001100.3(ACTA1):c.109G>T (p.Val37Leu), NM_001100.3(ACTA1):c.419C>G (p.Ala140Gly), NM_001100.3(ACTA1):c.809-2A>T, NM_001100.3(ACTA1):c.449C>G (p.Thr150Ser), NM_001100.3(ACTA1):c.1075A>C (p.IIe359Leu), NM_017825.3(ADPRHL2):c.235A>C (p.Thr79Pro), NM_152328.4(ADSS1):c.919del (p.IIe307fs), NM_000642.3(AGL):c.4506_4510del (p.Glu1502fs), NM_000642.3(AGL):c.2977_2982del (p.Phe993_Tyr994del), NM_015239.2(AGTPBP1):c.2849A>T (p.His950Leu), NM_015239.2(AGTPBP1):c.1960T>G (p.Tyr654Asp), NM_000030.3(AGXT):c.731T>C (p.IIe244Thr), NM_033087.4(ALG2):c.203T>G (p.Val68Gly), NM_004304.5(ALK):c.3521T>G (p.Phe1174Cys), NM_004304.5(ALK):c.3512T>A (p.IIe1171Asn), NM_004304.5(ALK):c.3733T>G (p.Phe1245Val), NM_004304.5(ALK):c.3734T>G (p.Phe1245Cys), NM_004304.5(ALK):c.3833A>C (p.Tyr1278Ser), NM_004304.5(ALK):c.3733T>A (p.Phe1245IIe), NM_001368809.2(AMPD2):c.102_103del (p.Gly35fs), NM_000481.3(AMT):c.959G>A (p.Arg320His), NM_000481.3(AMT):c.471+2T>C, NM_000481.3(AMT):c.125A>G (p.His42Arg), NM_000481.3(AMT):c.1033+2T>C, NM_000481.3(AMT):c.970_972del (p.Met324del), NM_173551.5(ANKS6):c.2512-2A>C, NM_173551.5(ANKS6):c.2054_2064del (p.His685fs), NM_173551.5(ANKS6):c.2370_2372del (p.Tyr790_Gln791delinsTer), NM_018075.5(ANO10):c.289del (p.Thr96_Met97insTer), NM_000486.5(AQP2):c.450T>A (p.Asp150Glu), NM_004815.4(ARHGAP29):c.1475C>A (p.Ser492Ter), NM_001162383.2(ARHGEF2):c.1545+1del, NM_000046.5(ARSB):c.1143-1G>C, NM_000046.5(ARSB):c.979C>T (p.Arg327Ter), NM_000046.5(ARSB):c.116_123del (p.Ala39fs), NM_000046.5(ARSB):c.1261G>T (p.Glu421Ter), NM_000046.5(ARSB):c.152T>C (p.Leu51 Pro), NM_000046.5(ARSB):c.962T>C (p.Leu321Pro), NM_000046.5(ARSB):c.1213+6T>C, NM_000046.5(ARSB):c.1142+2T>C, NM_000046.5(ARSB):c.1208C>G (p.Ser403Ter), NM_000046.5(ARSB):c.257del (p.Tyr86fs), NM_000046.5(ARSB):c.1336+2T>G, NM_000046.5(ARSB):c.238del (p.Val80fs), NM_000046.5(ARSB):c.743del (p.Pro248fs), NM_000046.5(ARSB):c.454C>T (p.Arg152Trp), NM_000046.5(ARSB):c.753C>G (p.Tyr251Ter), NM_015338.5(ASXL1):c.2100T>A (p.Tyr700Ter), NM_030632.3(ASXL3):c.6165del (p.Lys2055fs), NM_001692.4(ATP6V1B1):c.175-1G>C, NM_000490.5(AVP):c.287G>T (p.Gly96Val), NM_000490.5(AVP):c.294C>A (p.Cys98Ter), NM_000490.5(AVP):c.337G>T (p.Glu113Ter), NM_000490.5(AVP):c.200T>C (p.Val67Ala), NM_000490.5(AVP):c.161G>T (p.Gly54Val), NM_000490.5(AVP):c.61T>C (p.Tyr21His), NM_000490.5(AVP):c.143G>T (p.Gly48Val), NM_000054.6(AVPR2):c.838dup (p.Tyr280fs), NM_000054.6(AVPR2):c.424del (p.Cys142fs), NM_000054.6(AVPR2):c.819_821del (p.Leu274del), NM_000054.6(AVPR2):c.137T>A (p.IIe46Lys), NM_000054.6(AVPR2):c.395C>A (p.Ala132Asp), NM_000054.6(AVPR2):c.966del (p.Trp323fs), NM_000054.6(AVPR2):c.24del (p.Ala9fs), NM_000054.6(AVPR2):c.614A>G (p.Tyr205Cys), NM_003921.5(BCL10):c.345del (p.Gly116fs), NM_152743.4(BRAT1):c.294dup (p.Leu99fs), NM_032667.6(BSCL2):c.439-1G>C, NM_001256047.1(C19orf12):c.−10G>C, NM_001031726.3(C19orf12):c.197_199del, NM_001256047.1(C19orf12):c.362T>A (p.Leu121Gln), NM_001256047.1(C19orf12):c.161-2del, NM_001256047.1(C19orf12):c.−2C>T, NM_001127221.1(CACNA1A):c.1589_1590del (p.Phe530fs), NM_000718.4(CACNA1B):c.4857+1G>C, NM_000388.4(CASR):c.2165del (p.Asn722fs), NM_000388.4(CASR):c.823_824del (p.Asp275fs), NM_005188.3(CBL):c.1186T>C (p.Cys396Arg), NM_004064.4(CDKN1B):c.49_52del (p.Asp17fs), NM_004064.4(CDKN1B):c.−454_−451del, NM_004064.4(CDKN1B):c.251T>A (p.Leu84Ter), NM_014956.5(CEP164):c.32A>C (p.Gln11 Pro), NM_025114.3(CEP290):c.5707A>T (p.Glu1903Ter), NM_016122.3(CEP83):c.1532G>C (p.Arg511 Pro), NM_005198.4(CHKB):c.116C>A (p.Ser39Ter), NM_005198.4(CHKB):c.458dup (p.Leu153fs), NM_000751.3(CHRND):c.820_820+1del, NM_000751.3(CHRND):c.236T>A (p.IIe79Lys), NM_001145536.2(C17orf107):c.*1779del, NM_001145536.2(C17orf107):c.*327T>G, NM_001145536.2(C17orf107):c.*1399C>A, NM_000080.4(CHRNE):c.1033-2A>T, NM_000080.4(CHRNE):c.1030del (p.His344fs), NM_005199.5(CHRNG):c.301_309dup (p.Arg101_Pro103dup), NM_005199.5(CHRNG):c.401_402del (p.Pro134fs), NM_000083.3(CLCN1):c.1667T>A (p.IIe556Asn), NM_000083.3(CLCN1):c.394A>T (p.Ser132Cys), NM_000083.3(CLCN1):c.2330del (p.Gly777fs), NM_001843.4(CNTN1):c.871dup (p.Ser291fs), NM_001130103.1(COL13A1):c.523-1delG, NM_001130103.2(COL13A1):c.1173del (p.Leu392fs), NM_000088.3(COL1A1):c.1155+3_1155+6del, NM_000088.3(COL1A1):c.2049_2050del (p.Glu684fs), NM_000088.3(COL1A1):c.1635_1643del (p.543_545SPG[1]), NM_000091.4(COL4A3):c.3068_3069del (p.Pro1023fs), NM_001851.5(COL9A1):c.171del (p.Phe57fs), NM_001851.5(COL9A1):c.188del (p.Phe63fs), NM_000095.3(COMP):c.1156_1158del (p.Asn386del), NM_000095.3(COMP):c.1021_1026del (p.Glu341_Asp342del), NM_024876.4(COQ8B):c.532C>T (p.Arg178Trp), NM_024876.4(COQ8B):c.1356_1362del (p.Gln452fs), NM_024876.4(COQ8B):c.1199dup (p.His400fs), NM_024876.4(COQ8B):c.857A>G (p.Asp286Gly), NM_024876.4(COQ8B):c.645del (p.Phe215fs), NM_000096.4(CP):c.48del (p.Ala17fs), NM_001101426.4(CRPPA):c.722del (p.Leu241fs), NM_156039.3(CSF3R):c.1245del (p.Thr416fs), NM_156039.3(CSF3R):c.998-2A>T, NM_005213.4(CSTA):c.102_103del (p.Tyr35fs), NM_001031681.2(CTNS):c.519_520del (p.Tyr173_Ser174delinsTer), NM_001031681.2(CTNS):c.251del (p.Asn84fs), NM_014780.4(CUL7):c.263del (p.Val88fs), NM_001165928.3(DAG1):c.1257del (p.Thr421fs), NM_001165928.3(DAG1):c.310_311del (p.Leu104fs), NM_016356.5(DCDC2):c.349-2A>G, NM_016356.5(DCDC2):c.649A>T (p.Lys217Ter), NM_016356.5(DCDC2):c.942del (p.Gly315fs), NM_006182.4(DDR2):c.1912A>T (p.IIe638Phe), NM_001927.4(DES):c.1325C>T (p.Thr442IIe), NM_001927.4(DES):c.373A>T (p.Lys125Ter), NM_001927.4(DES):c.1213del (p.Tyr405fs), NM_001927.4(DES):c.1049G>C (p.Arg350Pro), NM_001927.4(DES):c.1043A>C (p.Gln348Pro), NM_001927.4(DES):c.639+4_639+5del, NM_001927.4(DES):c.1024A>G (p.Asn342Asp), NM_001927.4(DES):c.1034T>C (p.Leu345Pro), NM_001927.4(DES):c.1009G>C (p.Ala337Pro), NM_001927.4(DES):c.1154T>C (p.Leu385Pro), NM_001927.4(DES):c.1166A>C (p.Gln389Pro), NM_001927.4(DES):c.735+3A>G, NM_001927.4(DES):c.1069G>C (p.Ala357Pro), NM_001927.4(DES):c.1223del (p.Leu408fs), NM_003647.3(DGKE):c.728_731del (p.IIe242_Leu243insTer), NM_080916.3(DGUOK):c.749T>C (p.Leu250Ser), NM_080916.3(DGUOK):c.609_610del (p.Tyr204fs), NM_080916.3(DGUOK):c.591G>A (p.Gln197=), NM_001005360.2(DNM2):c.1862T>C (p.Leu621Pro), NM_173660.5(DOK7):c.55-1G>T, NM_173660.5(DOK7):c.548_551del (p.Phe183fs), NM_173660.5(DOK7):c.596del (p.IIe199fs), NM_173660.5(DOK7):c.1143dup (p.Glu382fs), NM_024422.6(DSC2):c.77del (p.IIe26fs), NM_024422.6(DSC2):c.473del (p.Gln158fs), NM_001943.5(DSG2):c.91del (p.Thr31fs), NM_001943.5(DSG2):c.307_308del (p.Val103fs), NM_001943.5(DSG2):c.2358del (p.Asp787fs), NM_015548.5(DST):c.13887del (p.Ala4630fs), NM_001347721.2(DYRK1A):c.1025del (p.Leu342fs), NM_001130987.2(DYSF):c.991G>T (p.Gly331Trp), NM_001130987.2(DYSF):c.5139del (p.Phe1713fs), NM_004092.4(ECHS1):c.74G>C (p.Arg25Pro), NM_001972.4(ELANE):c.136_141del (p.Ser46_Leu47del), NM_000117.2(EMD):c.266-2A>G, NM_001429.4(EP300):c.5571_5578del (p.Gly1860fs), NM_006494.4(ERF):c.397_407del (p.Pro133fs), NM_006494.4(ERF):c.1201_1202del (p.Lys401fs), NM_006494.4(ERF):c.1072_1073del (p.Pro358fs), NM_000125.3(ESR1):c.1607T>G (p.Leu536Arg), NM_000125.3(ESR1):c.1601T>A (p.Val534Glu), NM_000127.2(EXT1):c.1418-2A>G, NM_144670.6(A2ML1):c.4061+1G>C, NM_001605.2(AARS1):c.327_331del (p.Tyr109_Lys111delinsTer), NM_001287174.2(ABCC8):c.4273A>G (p.IIe1425Val), NM_001287174.2(ABCC8):c.3557C>A (p.Ser1186Tyr), NM_001287174.2(ABCC8):c.638T>G (p.Leu213Arg), NM_001287174.2(ABCC8):c.257T>G (p.Val86Gly), NM_001287174.2(ABCC8):c.215A>G (p.Asn72Ser), NM_001287174.2(ABCC8):c.3512del (p.Leu1171fs), NM_001287174.2(ABCC8):c.1752del (p.His584fs), NM_001287174.2(ABCC8):c.2124_2127del (p.Leu708_Thr709insTer), NM_001287174.2(ABCC8):c.2098_2099del (p.Thr700fs), NM_001287174.2(ABCC8):c.4519G>A (p.Glu1507Lys), NM_001287174.2(ABCC8):c.560T>A (p.Val187Asp), NM_001287174.2(ABCC8):c.1630+1G>T, NM_001287174.2(ABCC8):c.2147G>T (p.Gly716Val), NM_001287174.2(ABCC8):c.3133_3152del (p.Thr1045fs), NM_013227.3(ACAN):c.273del (p.Arg93fs), NM_013227.3(ACAN):c.1745del (p.Phe582fs), NM_013227.3(ACAN):c.5391del (p.Gln1798fs), NM_013227.3(ACAN):c.1425del (p.Val478fs), NM_174917.5(ACSF3):c.1718del (p.Phe573fs), NM_000020.2(ACVRL1):c.302del (p.Leu101fs), NM_000020.2(ACVRL1):c.271del (p.Asp91fs), NM_000020.2(ACVRL1):c.626-3C>G, NM_000022.4(ADA):c.890C>A (p.Pro297Gln), NM_001282225.2(ADA2):c.1358A>G (p.Tyr453Cys), NM_001282225.2(ADA2):c.506G>A (p.Arg169Gln), NM_001282225.2(ADA2):c.140G>C (p.Gly47Ala), NM_001282225.2(ADA2):c.973-2A>G, NM_001282225.2(ADA2):c.326C>A (p.Ala109Asp), NM_001282225.2(ADA2):c.336C>G (p.His112Gln), NM_001282225.2(ADA2):c.140G>T (p.Gly47Val), NM_005682.7(ADGRG1):c.1533T>G (p.Tyr511Ter), NM_005682.7(ADGRG1):c.739_745del (p.Gln247fs), NM_005682.7(ADGRG1):c.944_945dup (p.Val316fs), NM_005682.7(ADGRG1):c.272G>C (p.Cys91Ser), NM_005682.7(ADGRG1):c.1426C>T (p.Arg476Ter), NM_001134831.2(AH11):c.72_85del (p.Ser24fs), NM_000383.4(AIRE):c.247A>G (p.Lys83Glu), NM_000383.4(AIRE):c.239T>G (p.Val80Gly), NM_000383.4(AIRE):c.1103dup (p.Leu370fs), NM_000383.4(AIRE):c.1513del (p.Ala505fs), NM_000383.4(AIRE):c.1638A>T (p.Ter546Cys), NM_000383.4(AIRE):c.1116_1117del (p.Ala373fs), NM_000383.4(AIRE):c.809_810del (p.Glu270fs), NM_000383.4(AIRE):c.260del (p.Leu87fs), NM_000383.4(AIRE):c.415C>T (p.Arg139Ter), NM 000383.4(AIRE):c.1265del (p.Pro422fs), NM_000383.4(AIRE):c.205_208dup (p.Asp70fs), NM_000383.4(AIRE):c.254A>G (p.Tyr85Cys), NM_001625.4(AK2):c.656del (p.Val219fs), NM_005989.4(AKR1 D1):c.843del (p.Glu282fs), NM_024079.5(ALG8):c.1090C>T (p.Arg364Ter), NM_015120.4(ALMS1):c.1674del (p.Pro559fs), NM_021926.4(ALX4):c.385_394del (p.Cys129fs), NM_021926.4(ALX4):c.504del (p.Asp169fs), NM_152424.4(AMER1):c.1267del (p.Leu423fs), NM_001368809.2(AMPD2):c.2094C>G (p.Tyr698Ter), NM_001368809.2(AMPD2):c.2165T>G (p.Leu722Arg), NM_001368809.2(AMPD2):c.885C>A (p.Tyr295Ter), NM_001368809.2(AMPD2):c.1492del (p.Asp498fs), NM_213599.2(ANO5):c.2599del (p.Arg867fs), NM_032208.2(ANTXR1):c.1435-12A>G, NM_001272071.2(AP1S2):c.426+1G>T, NM_001272071.2(AP1S2):c.281del (p.Phe94fs), NM_000044.6(AR):c.521T>G (p.Leu174Ter), NM_001658.4(ARF1):c.103T>C (p.Tyr35His), NM_017519.2(ARID1B):c.5423_5427del (p.Leu1808fs), NM_018136.5(ASPM):c.10337del (p.Lys3446fs), NM_145178.4(ATOH7):c.146A>T (p.Glu49Val), NM_022089.4(ATP13A2):c.2561T>G (p.Met854Arg), NM_022089.4(ATP13A2):c.2552_2553del (p.Phe851fs), NM_006886.4(ATP5F1E):c.35A>G (p.Tyr12Cys), NM_001693.4(ATP6V1B2):c.1316_1318del (p.Val439del), NM_145691.4(ATPAF2):c.280T>A (p.Trp94Arg), NM_194318.4(B3GLCT):c.1098T>A (p.Tyr366Ter), NM_007255.3(B4GALT7):c.268del (p.Trp90fs), NM_006876.3(B4GAT1):c.1179_1181del (p.Asn393del), NM_004281.3(BAG3):c.580del (p.Ser194fs), NM_001123383.1(BCOR):c.4072-1G>T, NM_001123383.1(BCOR):c.2613del (p.Phe871fs), NM_001123383.1(BCOR):c.3268del (p.Asp1090fs), NM_001123383.1(BCOR):c.3410_3411del (p.Lys1137fs), NM_001123383.1(BCOR):c.3437_3445del (p.Glu1146_Thr1148del), NM_001079866.2(BCS1L):c.901T>A (p.Tyr301Asn), NM_006129.5(BMP1):c.2107G>C (p.Asp703His), NM_006129.5(BMP1):c.34G>C (p.Gly12Arg), NM_001202.6(BMP4):c.272C>G (p.Ser91Cys), NM_001204.7(BMPR2):c.1506del (p.Glu503fs), NM_001370658.1(BTD):c.248del (p.Lys83fs), NM_000061.2(BTK):c.1771del (p.Tyr591fs), NM_001199563.2(BVES):c.731_734del (p.Phe244fs), NM_001011551.3(C1GALT1C1):c.3G>C (p.Met1IIe), NM_001286577.1(C2CD3):c.3085T>G (p.Cys1029Gly), NM_000587.4(C7):c.405del (p.Asn136fs), NM_000067.3(CA2):c.21C>A (p.Tyr7Ter), NM_000067.3(CA2):c.120T>G (p.Tyr40Ter), NM_015215.4(CAMTA1):c.838del (p.Ser280fs), NM_000070.3(CAPN3):c.1914_1914+18del, NM_001366385.1(CARD14):c.467T>C (p.Leu156Pro), NM_001190442.1(CAST):c.298A>T (p.Lys100Ter), NM_001190442.1(CAST):c.1585del (p.Val529fs), NM_001001547.3(CD36):c.1237A>C (p.IIe413Leu), NM_001001547.3(CD36):c.1228_1239del (p.IIe410_IIe413del), NM_024529.4(CDC73):c.13_16del (p.Leu5fs), NM 024529.4(CDC73):c.245del (p.Asn82fs), NM_024529.4(CDC73):c.1052del (p.Pro351fs), NM_024529.4(CDC73):c.766_767del (p.Val256fs), NM_022124.6(CDH23):c.9058_9060del (p.Arg3020del), NM_022124.6(CDH23):c.4136G>T (p.Arg1379Leu), NM_033100.4(CDHR1):c.2246_2253del (p.Arg749fs), NM_018249.6(CDK5RAP2):c.140del (p.Val47fs), NM_018249.6(CDK5RAP2):c.1967del (p.Lys656fs), NM_001323289.2(CDKL5):c.214_216del (p.IIe72del), NM_001264.5(CDSN):c.164_167dup (p.Thr57fs), NM_001264.5(CDSN):c.175A>T (p.Lys59Ter), NM_001264.5(CDSN):c.746del (p.Gly249fs), NM_001807.5(CEL):c.2172del (p.Val725fs), NM_016343.4(CENPF):c.1026del (p.Glu342fs), NM_001194998.2(CEP152):c.3249del (p.Val1084fs), NM_000078.3(CETP):c.953_962del (p.Phe318fs), NM_000492.3(CFTR):c.1375_1383del (p.Ser459_Gly461del), NM_001271.4(CHD2):c.4081del (p.IIe1361fs), NM_017780.4(CHD7):c.3226_3227del (p.Lys1076fs), NM_002768.5(CHMP1A):c.88C>T (p.Gln30Ter), NM_000083.3(CLCN1):c.1444_1449del (p.Gly482_Gly483del), NM_001287.6(CLCN7):c.781A>T (p.IIe261 Phe), NM_000086.2(CLN3):c.370del (p.Tyr124fs), NM_006012.4(CLPP):c.440G>C (p.Cys147Ser), NM_006012.4(CLPP):c.433A>C (p.Thr145Pro), NM_001079878.2(CNGA3):c.1687_1688del (p.Lys563fs), NM_001297.5(CNGB1):c.2762_2765del (p.Tyr921fs), NM_001297.5(CNGB1):c.1120_1121+2del, NM_014141.6(CNTNAP2):c.3480_3481del (p.Gly1161fs), NM_014141.6(CNTNAP2):c.2964del (p.Cys989fs), NM_014141.6(CNTNAP2):c.3709del (p.Asp1237fs), NM_014141.6(CNTNAP2):c.1671-1G>T, NM_015386.3(COG4):c.1809del (p.Lys603fs), NM_000088.3(COL1A1):c.1812del (p.Gly605fs), NM_000088.3(COL1A1):c.2418del (p.Gly809fs), NM_000088.3(COL1A1):c.1657del (p.Thr553fs), NM_000088.3(COL1A1):c.3235G>A (p.Gly1079Ser), NM_000088.3(COL1A1):c.1002+2T>C, NM_000088.3(COL1A1):c.2172del (p.Gly725fs), NM_000088.3(COL1A1):c.697-1G>C, NM_000088.3(COL1A1):c.3207+1G>C, NM_000088.3(COL1A1):c.2534G>C (p.Gly845Ala), NM_000088.3(COL1A1):c.2128-1G>C, NM_000088.3(COL1A1):c.1654A>T (p.Lys552Ter), NM_000088.3(COL1A1):c.851G>C (p.Gly284Ala), NM_000088.3(COL1A1):c.3647A>G (p.Tyr1216Cys), NM_000088.3(COL1A1):c.608G>T (p.Gly203Val), NM_000088.3(COL1A1):c.589G>T (p.Gly197Cys), NM_000088.3(COL1A1):c.862G>T (p.Glu288Ter), NM_000088.3(COL1A1):c.1A>G (p.Met1Val), NM_000088.3(COL1A1):c.4358_4362del, NM_000088.3(COL1A1):c.2127+2T>A, NM_000088.3(COL1A1):c.802A>T (p.Arg268Ter), NM_000088.3(COL1A1):c.2028+2T>G, NM_000088.3(COL1A1):c.2525del (p.Gly842fs), NM_000088.3(COL1A1):c.2550del (p.Gly851fs), NM_000088.3(COL1A1):c.2684del (p.Pro895fs), NM_000088.3(COL1A1):c.4386del (p.Phe1463fs), NM_000088.3(COL1A1):c.1261del (p.Gln421fs), NM_000088.3(COL1A1):c.1121del (p.Gly374fs), NM_000088.3(COL1A1):c.1269del (p.Gly424fs), NM_000088.3(COL1A1):c.1379del (p.Pro460fs), NM_000088.3(COL1A1):c.2091_2092del (p.Ala699fs), NM_000088.3(COL1A1):c.2217del (p.Pro741fs), NM_000088.3(COL1A1):c.678del (p.Gly227fs), NM_000088.3(COL1A1):c.111_117del (p.IIe38fs), NM_000088.3(COL1A1):c.3788del (p.Lys1263fs), NM_000088.3(COL1A1):c.2139del (p.Ala714fs), NM_005502.4(ABCA1):c.2803A>C (p.Asn935His), NM_005502.4(ABCA1):c.1824del (p.Thr609fs), NM_005502.4(ABCA1):c.3343_3344del (p.Ser1115fs), NM_001089.3(ABCA3):c.4772A>C (p.Gln1591Pro), NM_001089.3(ABCA3):c.817_821del (p.Tyr273fs), NM_000350.3(ABCA4):c.1822T>A (p.Phe608IIe), NM_000350.3(ABCA4):c.2461T>C (p.Trp821Arg), NM_000350.3(ABCA4):c.2798A>T (p.Asn933IIe), NM_000350.3(ABCA4):c.67-1G>C, NM_000350.3(ABCA4):c.1239+1G>C, NM_000350.3(ABCA4):c.4354G>T (p.Glu1452Ter), NM_000350.3(ABCA4):c.2160+1G>T, NM_000350.3(ABCA4):c.3081T>G (p.Tyr1027Ter), NM_000350.3(ABCA4):c.2564G>A (p.Trp855Ter), NM_000350.3(ABCA4):c.1222C>T (p.Arg408Ter), NM_000350.3(ABCA4):c.5161_5162del (p.Thr1721fs), NM_000350.3(ABCA4):c.1937+1G>A, NM_000350.3(ABCA4):c.3085C>T (p.Gln1029Ter), NM_000350.3(ABCA4):c.4234C>T (p.Gln1412Ter), NM_000350.3(ABCA4):c.4253+5G>T, NM_000350.3(ABCA4):c.5196+1G>A, NM_000350.3(ABCA4):c.6386+2C>G, NM_000350.3(ABCA4):c.161G>T (p.Cys54Phe), NM_000350.3(ABCA4):c.296dup (p.Asn99fs), NM_000350.3(ABCA4):c.1760+2T>G, NM_000350.3(ABCA4):c.5196+2T>C, NM_000350.3(ABCA4):c.6609C>A (p.Tyr2203Ter), NM_000350.3(ABCA4):c.571-2A>G, NM_000350.3(ABCA4):c.1018T>G (p.Tyr340Asp), NM_000350.3(ABCA4):c.2300T>A (p.Val767Asp), NM_000350.3(ABCA4):c.5316G>A (p.Trp1772Ter), NM_000350.3(ABCA4):c.5912T>G (p.Leu1971Arg), NM_022436.3(ABCG5):c.978del (p.Glu326fs), NM_022437.3(ABCG8):c.320C>G (p.Ser107Ter), NM_022437.3(ABCG8):c.1787T>G (p.Leu596Arg), NM_022437.3(ABCG8):c.547del (p.Gln183fs), NM_016006.6(ABHD5):c.594dup (p.Arg199fs), NM_016006.6(ABHD5):c.389A>C (p.Gln130Pro), NM_016006.6(ABHD5):c.98C>G (p.Ser33Ter), NM_013227.3(ACAN):c.5061T>A (p.Ser1687Arg), NM_001611.5(ACP5):c.831_833del (p.Tyr278del), NM_001611.5(ACP5):c.816dup (p.Lys273fs), NM_000022.4(ADA):c.1079-15T>A, NM_000022.4(ADA):c.911T>G (p.Leu304Arg), NM_000022.4(ADA):c.646G>A (p.Gly216Arg), NM_000022.4(ADA):c.7C>T (p.Gln3Ter), NM_000022.4(ADA):c.302G>A (p.Arg101Gln), NM_000022.4(ADA):c.478+1G>A, NM_000022.4(ADA):c.956_960del (p.Glu319fs), NM_139025.4(ADAMTS13):c.581G>T (p.Gly194Val), NM_006796.2(AFG3L2):c.2011G>A (p.Gly671Arg), NM_006796.2(AFG3L2):c.1997T>G (p.Met666Arg), NM_006796.2(AFG3L2):c.1996A>G (p.Met666Val), NM_006796.2(AFG3L2):c.1295A>C (p.Asn432Thr), NM_018238.4(AGK):c.672C>G (p.Tyr224Ter), NM_018238.4(AGK):c.306T>G (p.Tyr102Ter), NM_018238.4(AGK):c.1170T>G (p.Tyr390Ter), NM_018238.4(AGK):c.3G>C (p.Met1IIe), NM_003977.4(AIP):c.66_71del (p.Gly23_Glu24del), NM_003977.4(AIP):c.543del (p.IIe182fs), NM_003977.4(AIP):c.286_287del (p.Val96fs), NM_003977.4(AIP):c.404del (p.His135fs), NM_003977.4(AIP):c.521_525del (p.Glu174fs), NM_003977.4(AIP):c.854_857del (p.Gn285fs), NM_003977.4(AIP):c.245_249del (p.Glu82fs), NM_152327.5(AK7):c.2018T>G (p.Leu673Arg), NM_002860.4(ALDH18A1):c.359T>C (p.Val120Ala), NM_002860.4(ALDH18A1):c.1994G>T (p.Arg665Leu), NM_000382.3(ALDH3A2):c.521del (p.Leu174fs), NM_000382.3(ALDH3A2):c.371_373del (p.Gly124del), NM_000382.3(ALDH3A2):c.1100del (p.Asn367fs), NM 000382.3(ALDH3A2):c.231del (p.Glu77fs), NM_000382.3(ALDH3A2):c.943C>T (p.Pro315Ser), NM_000382.3(ALDH3A2):c.1309A>T (p.Lys437Ter), NM_001080.3(ALDH5A1):c.1226G>A (p.Gly409Asp), NM_001080.3(ALDH5A1):c.612G>A (p.Trp204Ter), NM_001080.3(ALDH5A1):c.278G>T (p.Cys93Phe), NM_000478.6(ALPL):c.360_361del (p.Val121fs), NM_001368809.2(AMPD2):c.157del (p.Cys53fs), NM_001142446.2(ANK1):c.1801-2A>C, NM_001142446.2(ANK1):c.1618dup (p.Leu540fs), NM_001142446.2(ANK1):c.127-39474_127-39473del, NM_018075.5(ANO10):c.1669-2A>T, NM_018075.5(ANO10):c.1529T>G (p.Leu510Arg), NM_018075.5(ANO10):c.1604del (p.Ala534_Leu535insTer), NM_018075.5(ANO10):c.96del (p.Glu33fs), NM_001253852.3(AP4B1):c.313del (p.Ala105fs), NM_004722.4(AP4M1):c.32del (p.Lys11fs), NM_014855.3(AP5Z1):c.1413_1426del (p.Leu473fs), NM_001655.5(ARCN1):c.633del (p.Val212fs), NM_004315.6(ASAH1):c.35G>C (p.Arg12Pro), NM_001198800.3(ASCC1):c.157dup (p.Glu53fs), NM_000049.3(ASPA):c.454T>C (p.Cys152Arg), NM_000049.3(ASPA):c.746A>T (p.Asp249Val), NM_001321336.1(SPATA22):c.−74+19047_-74+19048del, NM_001321336.1(SPATA22):c.−74+14439_-74+14440del, NM_018263.6(ASXL2):c.2424del (p.Thr809fs), NM_018263.6(ASXL2):c.2081dup (p.Gly696fs), NM_018263.6(ASXL2):c.2971_2974del (p.Gly991fs), NM_018263.6(ASXL2):c.2472del (p.Ser825fs), NM_018263.6(ASXL2):c.1225_1228del (p.Pro409fs), NM_015915.4(ATL1):c.470T>G (p.Leu157Trp), NM_015915.4(ATL1):c.773A>G (p.His258Arg), NM_001127713.1(ATL1):c.1519dupA (IIe507Asnfs), NM_015915.4(ATL1):c.596T>A (p.Leu199Gln), NM_001184.4(ATR):c.4641+1G>T, NM_002973.4(ATXN2):c.176_190del (p.Val59_Ser63del), NM_001478.5(B4GALNT1):c.1298A>C (p.Asp433Ala), NM_001478.5(B4GALNT1):c.395del (p.Pro132fs), NM_004656.4(BAP1):c.1358_1359del (p.Lys453fs), NM_004656.4(BAP1):c.517dup (p.Tyr173fs), NM 004656.4(BAP1):c.519T>G (p.Tyr173Ter), NM_004656.4(BAP1):c.544G>T (p.Glu182Ter), NM_004656.4(BAP1):c.1379C>A (p.Ser460Ter), NM_004656.4(BAP1):c.181A>T (p.Lys61Ter), NM_004656.4(BAP1):c.1975A>T (p.Lys659Ter), NM_004656.4(BAP1):c.1938T>A (p.Tyr646Ter), NM_004656.4(BAP1):c.1379C>G (p.Ser460Ter), NM_004656.4(BAP1):c.1654del (p.Asp552fs), NM 004656.4(BAP1):c.1305del (p.Gln436fs), NM_004656.4(BAP1):c.78_79del (p.Val27fs), NM_004656.4(BAP1):c.644del (p.Gly215fs), NM_004656.4(BAP1):c.1416del (p.Ser473fs), NM_001164405.1(BHLHA9):c.218G>C (p.Arg73Pro), NM_001164405.1(BHLHA9):c.211A>G (p.Asn71Asp), NM_001003800.2(BICD2):c.1502G>C (p.Arg501 Pro), NM_001003800.2(BICD2):c.1523A>C (p.Lys508Thr), NM_001003800.2(BICD2):c.563A>C (p.Asn188Thr), NM_001003800.2(BICD2):c.2321A>G (p.Glu774Gly), NM_152743.4(BRAT1):c.1313_1314del (p.Gn438fs), NM_138425.4(C12orf57):c.152T>A (p.Leu51Gln), NM_152269.5(C12orf65):c.282+2T>A, NM_152269.5(C12orf65):c.413_417del (p.Lys138fs), NM_000719.7(CACNA1C):c.1114-304G>C, NM_005184.4(CALM3):c.286G>C (p.Asp96His), NM_001198868.2(CAPN1):c.407del (p.Pro136fs), NM_033337.2(CAV3):c.84C>A (p.Asp28Glu), NM_014008.5(CCDC22):c.49A>G (p.Thr17Ala), NM_001791.4(CDC42):c.247T>C (p.Ser83Pro), NM_016343.4(CENPF):c.2734G>T (p.Glu912Ter), NM_016343.4(CENPF):c.574-2A>C, NM_018451.5(CENPJ):c.3302-1G>C, NM_018451.5(CENPJ):c.1404_1407del (p.Ser469fs), NM_018451.5(CENPJ):c.1882del (p.Ala628fs), NM_018451.5(CENPJ):c.1850_1851del (p.Pro617fs), NM_018451.5(CENPJ):c.1339A>T (p.Lys447Ter), NM_018451.5(CENPJ):c.3936_3939del (p.His1313fs), NM_004928.3(CFAP410):c.671T>C (p.Leu224Pro), NM_004928.3(CFAP410):c.103del (p.IIe35fs), NM_004928.3(CFAP410):c.643-23A>T, NM_001164496.2(CFAP44):c.1769T>A (p.Leu590Gln), NM_213720.3(CHCHD10):c.197G>T (p.Gly66Val), NM_023077.3(COA7):c.446G>T (p.Ser149IIe), NM_023077.3(COA7):c.17A>G (p.Asp6Gly), NM_023077.3(COA7):c.247+1G>T, NM_001854.4(COL11A1):c.781-70T>G, NM_001854.4(COL11A1):c.4396G>T (p.Glu1466Ter), NM_001854.4(COL11A1):c.1874G>T (p.Gly625Val), NM_032888.4(COL27A1):c.2089G>C (p.Gly697Arg), NM_001844.5(COL2A1):c.3517G>C (p.Gly1173Arg), NM_001844.5(COL2A1):c.3655G>C (p.Asp1219His), NM_001844.5(COL2A1):c.1510G>T (p.Gly504Cys), NM_001844.5(COL2A1):c.2035A>T (p.Lys679Ter), NM_001844.5(COL2A1):c.3642del (p.Gly1215fs), NM_001844.5(COL2A1):c.609+4del, NM_000090.3(COL3A1):c.536del (p.Pro179fs), NM_000094.3(COL7A1):c.3840del (p.Gly1281fs), NM_001852.4(COL9A2):c.843_846+4del, NM_001079846.1(CREBBP):c.2T>A (p.Met1 Lys), NM_001079846.1(CREBBP):c.2008_2009del (p.Leu670fs), NM_001079846.1(CREBBP):c.1912del (p.Gln638fs), NM_001079846.1(CREBBP):c.1476del (p.Asn492fs), NM_001079846.1(CREBBP):c.299del (p.Gly100fs), NM_001079846.1(CREBBP):c.4575del (p.Lys1527fs), NM_001079846.1(CREBBP):c.3723-2A>T, NM_001079846.1(CREBBP):c.827_828dup (p.Gly277fs), NM_001079846.1(CREBBP):c.4546A>T (p.Lys1516Ter), NM_000350.3(ABCA4):c.3098del (p.Lys1033fs), NM_000350.3(ABCA4):c.1225del (p.Arg409fs), NM_000350.3(ABCA4):c.2566T>A (p.Tyr856Asn), NM_000350.3(ABCA4):c.4773+3A>G, NM_000350.3(ABCA4):c.4253+5G>A, NM_000350.3(ABCA4):c.4919G>A (p.Arg1640Gln), NM_000350.3(ABCA4):c.2927del (p.Leu976fs), NM_000350.3(ABCA4):c.1848del (p.Glu616fs), NM_000350.3(ABCA4):c.2537A>T (p.Asp846Val), NM_000350.3(ABCA4):c.5175dup (p.Thr1726fs), NM_003742.4(ABCB11):c.1416T>A (p.Tyr472Ter), NM_003742.4(ABCB11):c.3767dup (p.Val1257fs), NM_003742.4(ABCB11):c.150+3A>C, NM_003742.4(ABCB11):c.908+1del, NM_003742.4(ABCB11):c.1445A>G (p.Asp482Gly), NM_018849.3(ABCB4):c.394_400del (p.Tyr132fs), NM_018849.3(ABCB4):c.3077del (p.Lys1026fs), NM_001171.5(ABCC6):c.3712G>C (p.Asp1238His), NM_001171.5(ABCC6):c.3775del (p.Trp1259fs), NM_001171.5(ABCC6):c.3892G>T (p.Val1298Phe), NM_001171.5(ABCC6):c.1064T>G (p.Leu355Arg), NM_001171.5(ABCC6):c.4420A>T (p.Lys1474Ter), NM_001171.5(ABCC6):c.4060G>C (p.Gly1354Arg), NM_001171.5(ABCC6):c.2432C>G (p.Thr811Arg), NM_001171.5(ABCC6):c.1563G>C (p.Glu521Asp), NM_001171.5(ABCC6):c.2419C>G (p.Arg807Gly), NM_001171.5(ABCC6):c.3907G>C (p.Ala1303Pro), NM_001171.5(ABCC6):c.3883-10C>G, NM_001171.5(ABCC6):c.3306+5G>C, NM_001171.5(ABCC6):c.3208G>C (p.Ala1070Pro), NM_001171.5(ABCC6):c.2974G>C (p.Gly992Arg), NM_001171.5(ABCC6):c.2488G>C (p.Ala830Pro), NM_001171.5(ABCC6):c.2458G>C (p.Ala820Pro), NM_001171.5(ABCC6):c.4041G>C (p.Gln1347His), NM_001171.5(ABCC6):c.1258C>G (p.Leu420Val), NM_001171.5(ABCC6):c.1194C>G (p.Ser398Arg), NM_001171.5(ABCC6):c.1176G>C (p.Lys392Asn), NM_001171.5(ABCC6):c.1091C>G (p.Thr364Arg), NM_001171.5(ABCC6):c.951C>G (p.Ser317Arg), NM_001171.5(ABCC6):c.681C>G (p.Tyr227Ter), NM_001171.5(ABCC6):c.220-1G>C, NM_001171.5(ABCC6):c.130C>G (p.Leu44Val), NM_001171.5(ABCC6):c.113G>C (p.Trp38Ser), NM_001171.5(ABCC6):c.1363G>C (p.Ala455Pro), NM_001171.5(ABCC6):c.1388T>A (p.Leu463His), NM_001171.5(ABCC6):c.1505A>T (p.Lys502Met), NM_001171.5(ABCC6):c.3880_3882del (p.Lys1294del), NM_001171.5(ABCC6):c.3506+2_3506+5del, NM_001171.5(ABCC6):c.3876_3882+1del, NM_001171.5(ABCC6):c.3912del (p.Lys1305fs), NM_001171.5(ABCC6):c.4254del (p.Lys1419fs), NM_001171.5(ABCC6):c.4335del (p.Ser1446fs), NM_001171.5(ABCC6):c.4434del (p.Glu1479fs), NM_001171.5(ABCC6):c.179_187del (p.Arg60_Tyr62del), NM_001171.5(ABCC6):c.177_181del (p.Arg60fs), NM_001171.5(ABCC6):c.105del (p.Val37fs), NM_001171.5(ABCC6):c.2248-2_2248-1del, NM_001171.5(ABCC6):c.2248-12_2248-11del, NM_001171.5(ABCC6):c.1674del (p.Glu559fs), NM_001171.5(ABCC6):c.998+2_998+3del, NM_001171.5(ABCC6):c.998+2del, NM_001171.5(ABCC6):c.2383del (p.Val795fs), NM_001171.5(ABCC6):c.2416-1_2416del, NM_001171.5(ABCC6):c.2784_2787del (p.Gly928_Arg929insTer), NM_001171.5(ABCC6):c.220_222del, NM_001171.5(ABCC6):c.280del (p.IIe94fs), NM_001171.5(ABCC6):c.373G>T (p.Glu125Ter), NM_001171.5(ABCC6):c.654G>T (p.Trp218Cys), NM_001171.5(ABCC6):c.724G>T (p.Glu242Ter), NM_001171.5(ABCC6):c.2858T>A (p.Leu953His), NM_001171.5(ABCC6):c.1484T>A (p.Leu495His), NM_001171.5(ABCC6):c.2252T>A (p.Met751Lys), NM_001171.5(ABCC6):c.1318T>G (p.Cys440Gly), NM_001171.5(ABCC6):c.3188T>G (p.Leu1063Arg), NM_001171.5(ABCC6):c.3145T>G (p.Ser1049Ala), NM_001171.5(ABCC6):c.2542del (p.Val848fs), NM_001171.5(ABCC6):c.3798del (p.Glu1266fs), NM_001171.5(ABCC6):c.4306_4312del (p.Thr1436fs), NM_001171.5(ABCC6):c.4318del (p.Met1440fs), NM_001171.5(ABCC6):c.1999del (p.Ala667fs), NM_001171.5(ABCC6):c.1519del (p.Glu507fs), NM_001171.5(ABCC6):c.3490C>T (p.Arg1164Ter), NM_001171.5(ABCC6):c.4182del (p.Lys1394fs), NM_001171.5(ABCC6):c.2018T>C (p.Leu673Pro), NM_001171.5(ABCC6):c.3143_3145del (p.Phe1048del), NM_000789.4(ACE):c.1319_1322del (p.Leu440fs), NM_004035.7(ACOX1):c.1851del (p.Gly618fs), NM_001105.5(ACVR1):c.617G>A (p.Arg206His), NM_000020.2(ACVRL1):c.1433C>A (p.Ala478Asp), NM_000020.2(ACVRL1):c.1390del (p.Gly463_Leu464insTer), NM_000020.2(ACVRL1):c.1280A>T (p.Asp427Val), NM_000020.2(ACVRL1):c.1460A>C (p.Lys487Thr), NM_000020.2(ACVRL1):c.593T>A (p.Val198Glu), NM_000020.2(ACVRL1):c.1388del (p.Gly463fs), NM_000020.2(ACVRL1):c.1385C>G (p.Ser462Ter), NM_001110.4(ADAM10):c.429T>A (p.Tyr143Ter), NM_032119.4(ADGRV1):c.14973-2A>G, NM_032119.4(ADGRV1):c.11253C>G (p.Tyr3751Ter), NM_032119.4(ADGRV1):c.2870dup (p.Asn957fs), NM_032119.4(ADGRV1):c.17662del (p.Ser5888fs), NM_032119.4(ADGRV1):c.1701del (p.Leu568fs), NM_032119.4(ADGRV1):c.5643del (p.Tyr1882fs), NM_032119.4(ADGRV1):c.7374_7375del (p.Glu2459fs), NM_032119.4(ADGRV1):c.8737del (p.Val2913fs), NM_032119.4(ADGRV1):c.12798T>A (p.Tyr4266Ter), NM_000030.3(AGXT):c.466G>A (p.Gly156Arg), NM_000030.3(AGXT):c.596-2A>G, NM_000030.3(AGXT):c.2T>C (p.Met1Thr), NM_000030.3(AGXT):c.447_454del (p.Leu151fs), NM_000030.3(AGXT):c.198C>G (p.Tyr66Ter), NM_000030.3(AGXT):c.680+1G>C, NM_000030.3(AGXT):c.1076T>C (p.Leu359Pro), NM_000030.3(AGXT):c.1151T>C (p.Leu384Pro), NM_000030.3(AGXT):c.1014C>G (p.Tyr338Ter), NM_000030.3(AGXT):c.947T>C (p.Leu316Pro), NM_000030.3(AGXT):c.851T>C (p.Leu284Pro), NM_000030.3(AGXT):c.823A>C (p.Ser275Arg), NM_000030.3(AGXT):c.806T>C (p.Leu269Pro), NM_000030.3(AGXT):c.783T>A (p.His261Gin), NM_000030.3(AGXT):c.776+1G>C, NM_000030.3(AGXT):c.757T>C (p.Cys253Arg), NM_000030.3(AGXT):c.680+2T>A, NM_000030.3(AGXT):c.727G>C (p.Asp243His), NM_000030.3(AGXT):c.680+5G>C, NM_000030.3(AGXT):c.725dup (p.Asp243fs), NM_000030.3(AGXT):c.698G>T (p.Arg233Leu), NM_000030.3(AGXT):c.777-2A>G, NM_000030.3(AGXT):c.846G>C (p.Gln282His), NM_000030.3(AGXT):c.853G>T (p.Glu285Ter), NM_000030.3(AGXT):c.893T>C (p.Leu298Pro), NM_000030.3(AGXT):c.942+1G>T, NM_000030.3(AGXT):c.661T>C (p.Ser221Pro), NM_000030.3(AGXT):c.481G>C (p.Gly161Arg), NM_000030.3(AGXT):c.466G>C (p.Gly156Arg), NM_000030.3(AGXT):c.449T>C (p.Leu150Pro), NM_000030.3(AGXT):c.371A>C (p.His124Pro), NM_000030.3(AGXT):c.358+1G>T, NM_000030.3(AGXT):c.349G>T (p.Glu117Ter), NM_000030.3(AGXT):c.583A>C (p.Met195Leu), NM_000030.3(AGXT):c.577dup (p.Leu193fs), NM_000030.3(AGXT):c.584T>G (p.Met195Arg), NM_000030.3(AGXT):c.74T>G (p.Leu25Arg), NM_000030.3(AGXT):c.628G>C (p.Ala210Pro), NM_000030.3(AGXT):c.614C>A (p.Ser205Ter), NM_000030.3(AGXT):c.612C>A (p.Tyr204Ter), NM_000030.3(AGXT):c.605T>A (p.IIe202Asn), NM_000030.3(AGXT):c.603C>A (p.Asp201Glu), NM_000030.3(AGXT):c.3G>T (p.Met1 lie), NM_000030.3(AGXT):c.242C>A (p.Ser81Ter), NM 000030.3(AGXT):c.215dup (p.Asn72fs), NM_000030.3(AGXT):c.209C>A (p.Thr70Asn), NM_000030.3(AGXT):c.187G>C (p.GIy63Arg), NM_000030.3(AGXT):c.244G>C (p.GIy82Arg), NM_000030.3(AGXT):c.77T>C (p.Leu26Pro), NM_000030.3(AGXT):c.32_33del (p.Pro11fs), NM_000030.3(AGXT):c.570del (p.Thr191fs), NM_000030.3(AGXT):c.642_645del (p.Pro215fs), NM_000030.3(AGXT):c.679_680+2del, NM_000030.3(AGXT):c.445del (p.Val149fs), NM_000030.3(AGXT):c.83del (p.Pro28fs), NM_000030.3(AGXT):c.126del (p.Leu43fs), NM_000030.3(AGXT):c.276del (p.Asn92fs), NM_000030.3(AGXT):c.983_988del (p.Ala328_Tyr330delinsAsp), NM_000030.3(AGXT):c.919del (p.Leu307fs), NM_000030.3(AGXT):c.744del (p.Asn249fs), NM_000030.3(AGXT):c.834dei (p.IIe279fs), NM_000030.3(AGXT):c.997A>T (p.Arg333Ter), NM_000030.3(AGXT):c.283_285dup (p.Glu95dup), NM_000030.3(AGXT):c.167T>A (p.IIe56Asn), NM_000030.3(AGXT):c.416_418del (p.Val139del), NM_000030.3(AGXT):c.673_676del (p.Lys225fs), NM_000030.3(AGXT):c.847-3C>G, NM_000030.3(AGXT):c.613T>C (p.Ser205Pro), NM_000030.3(AGXT):c.846+1G>T, NM_000030.3(AGXT):c.33dup (p.Lys12fs), NM_001625.4(AK2):c.25G>T (p.Glu9Ter), NM_001625.4(AK2):c.453del (p.Tyr152fs), NM_001625.4(AK2):c.523del (p.Arg175fs), and NM_001625.4(AK2):c.118del (p.Cys40fs).

### Nucleic Acid Modifications

One or more chemical or nucleic acid modifications can be applied to the pegRNAs described herein. Exemplary nucleic acid modifications include, but are not limited to, nucleobase modifications, sugar modifications, inter-sugar linkage modifications, conjugates (e.g., ligands), and any combinations thereof. Nucleic acid modifications also include unnatural, or degenerate nucleobases.

Exemplary modified nucleobases include, but are not limited to, inosine, xanthine, hypoxanthine, nebularine, isoguanosine, tubercidin, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, a modified nucleobase can be selected from the group consisting of: inosine, xanthine, hypoxanthine, nebularine, isoguanosine, tubercidin, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 5-ethynyl-2′-deoxyuridine, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2-(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2-(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2-(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidin, isoguanosine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

In some embodiments, a nucleic acid modification can include a non-natural or modified nucleobase.

Exemplary sugar modified nucleotides include, but are not limited to, 2′-O-methyl (2′-OMe) nucleotides, 2′-fluoro (2′-F) nucleotides, 3′-fluoro nucleotides, 3′-OMe nucleotides, bridged nucleic acid (BNA) nucleotides (e.g., 2′-0,4′-C-methylene (locked nucleic acid, LNA) nucleotides, 2′-0,4′-C-ethylene (locked nucleic acid, ENA) nucleotides, 5′-methyl-BNA, cEt BNA, cMOE BNA, oxy amino BNA and vinyl-carbo BNA), anhydrohexitol (1,5-anhydrohexitol nucleic acid, HNA) nucleotides, cyclohexene (Cyclohexene nucleic acid, CeNA) nucleotides, 2′-methoxyethyl (2′-MOE) nucleotides, 2′-O-allyl nucleotides, 2′-C-allyl ribose nucleotides, 2′-O—N-methylacetamido (2′-O-NMA) nucleotides, a 2′-O-dimethylaminoethoxyethyl (2′-O-DMAEOE) nucleotides, 2′-O-aminopropyl (2′-O-AP) nucleotides, 2′-F arabinose (2′-ara-F) nucleotides, threose (Threose nucleic acid, TNA) nucleotides, and acyclic nucleotides (e.g., peptide nucleic acid (PNA), unlocked nucleic acids (UNA), 2,3-dihydroxylpropyl (glycol nucleic acid, GNA)), and 2′-deoxy (2′-H); a modified internucleoside linkage; a non-natural or modified nucleobase; or a combination thereof.

In some embodiments, a nucleic acid modification can include replacement or modification of an inter-sugar linkage, i.e., a modified internucleoside linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH₂—C(═O)—N(H)-5′) and amide-4 (3′-CH₂—N(H)—C(═O)-5′), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methylenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′).

Backbone modifications such as phosphorothioates modify the charge on the phosphate backbone and can aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387). Modifications of sugars, such as 2′-O-methyl (2′-OMe), 2′-F, and locked nucleic acid (LNA), can enhance both base pairing and nuclease resistance (see, e.g., Allerson et al., “Fully 2′-modified oligonucleotide duplexes with improved in vitro potency and stability compared to unmodified small interfering RNA,” J Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet. 2012 Aug. 20; 3: 154). Additionally, the guide nucleic acid is amenable to both 5′ and 3′ end conjugations with a variety of functional moieties including, but not limited to, targeting ligands, fluorescent dyes, polyethylene glycol, or proteins.

In some embodiments of any one of the aspects described herein, each modified internucleoside linkage can be selected independently from the group consisting of phosphorothioates (R, S, or racemic), phosphorodithioates, methylenemethylimino (MMI, 3′-CH₂—N(CH₃)—O-5′), phosphotriesters, alkylphosphonates (e.g., methylphosphonates), phosphoramidate, methylenemethylimino (—CH₂—N(CH₃)—O—CH₂—), thiodiester (—O—C(O)—S—), thionocarbamate (—O—C(O)(NH)—S—), siloxane (—O—Si(H)₂—O— and dialkylsiloxane), N,N′-dimethylhydrazine (—CH₂—N(CH₃)—N(CH₃)—), amide-3 (3′-CH₂—C(═O)—N(H)-5′), amide-4 (3′-CH₂—N(H)—C(═O)-5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′), C3′-CH₂—NH—NH—C5′, 3′-NHP(O)(OCH₃)—O-5′, 3′-NHP(O)(OCH₃)—O-5′), 2′->5′ internucleoside linkages, 2′->3′ internucleoside linkages, 3′->3′ internucleoside linkages, 5′->5′ internucleoside linkages, and imidophosphoramidate (“imidp”) linkage

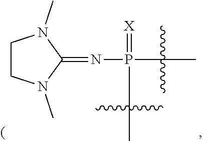

where X is O or S, preferably X is O).

A summary of chemical modifications amenable to the pegRNAs described herein can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

Nucleic Acids Encoding pegRNA, napDNAbp and/or a Nucleic Acid Modifying Enzyme

In another aspect provided herein is a nucleic acid encoding a pegRNA, napDNAbp and/or a nucleic acid modifying enzyme described herein. The skilled person will understand that, due to the degeneracy of the genetic code, a given polypeptide or protein can be encoded by different nucleic acids. These “variants” are encompassed herein.

### Vector

In some embodiments, a nucleic acid encoding a pegRNA, napDNAbp and/or a nucleic acid modifying enzyme described herein is comprised in a vector. In some of the aspects described herein, a nucleic acid sequence encoding pegRNA, napDNAbp and/or a nucleic acid modifying enzyme described herein, a genomic component comprised by a VLP, a given viral polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, a vector is recombinant, e.g., it comprises sequences originating from at least two different sources. In some embodiments of any of the aspects, a vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments, the vector is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments, the expression system is an organism other than the source of the native/wild-type sequence (or a cell obtained from such organism). In some embodiments, the vector and/or nucleic acid sequence described herein is codon-optimized for expression in a mammal or mammalian cell, e.g., a mouse, a murine cell, or a human cell. In some embodiments, the vector and/or nucleic acid sequence described herein is codon-optimized for expression in a human cell.

A vector can be an expression vector. As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Some non-limiting examples of a viral vector include, but are not limited to. an AAV vector, an adenovirus vector, a lentivirus vector, a retrovirus vector, a herpesvirus vector, an alphavirus vector, a poxvirus vector, a baculovirus vector, and a chimeric virus vector.

### Subject

As used herein, the term “subject” or “patient” refers to any organism to which a a pegRNA, a napDNAbp and/or nucleic acid modifying enzyme disclosed herein can be administered, e.g., for experimental, diagnostic, and/or therapeutic purposes. Typical subjects include animals (e.g., mammals such as mice, rats, rabbits, non-human primates, and humans) and/or plants. Usually, the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to with domesticated animals and/or pets.

In some embodiments, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species. In some embodiments, the subject can be of European ancestry. In some embodiments, the subject can be of African American ancestry. In some embodiments, the subject can be of Asian ancestry.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

A subject can be one who has been previously diagnosed with or identified as suffering from or having a condition in need of treatment. Alternatively, a subject can also be one who has not been previously diagnosed. A “subject in need” of testing for a particular condition can be a subject having that condition, diagnosed as having that condition, or at risk of developing that condition.

Some exemplary embodiments of the various aspects described herein can be described by one or more of the following numbered embodiments:

Embodiment 1: A prime editing guide RNA (pegRNA) comprising: (a) a spacer domain comprising a sequence substantially complementary to a region of a first strand (non-edit strand) of a double-stranded target nucleic acid; (b) a gRNA core domain capable of associating with a nucleic acid programmable DNA binding protein (napDNAbp); (c) a nucleic acid synthesis template domain (RTT) comprising an edit template domain comprising a sequence having one or more nucleotide changes compared to a second strand (edit strand) of the double-stranded target nucleic acid, and optionally the nucleic acid synthesis template domain further comprises an homology arm domain comprising a sequence substantially complementary the second strand of the double-stranded target nucleic acid; and (d) a primer binding site (PBS) comprising a sequence substantially complementary to a region upstream of the region complementary to the nucleic acid synthesis template domain in the second strand of the double-stranded target nucleic acid, and wherein: the pegRNA is circularized; or (ii) the pegRNA comprises a first portion of the gRNA core domain at one of the 5′-end or the 3′-end, and a second portion of the gRNA core domain at the other of the 5′-end or the 3′-end, and wherein the first and second portions together form the gRNA core domain; or (iii) the pegRNA comprises a first ribozyme and a first ligation sequence positioned 3′ to the first ribozyme at 5′-end, and a second ribozyme and a second ligation sequence positioned 3′ to the second ribozyme at the 3′-end, and wherein a portion of the first ligation sequence is complementary to a portion of the first ribozyme and a portion of the second ligation sequence is complementary to a portion of the second ribozyme, wherein a portion of the first ligation sequence is complementary to a portion of the second ligation sequence; and wherein the portion of the first ligation sequence complementary to the portion of the first ribozyme is complementary to the portion of the second ligation sequence complementary to the portion of the second ribozyme.

Embodiment 2: The pegRNA of Embodiment 1, wherein: the spacer domain is 5′ of the gRNA core domain, the gRNA core domain is 5′ of the nucleic acid synthesis template domain, and the nucleic acid synthesis template domain is 5′ of the primer binding site.

Embodiment 3: The pegRNA of any one of Embodiments 1-2, wherein: a first portion of the gRNA core domain is 5′ of the nucleic acid synthesis template domain, the nucleic acid synthesis template domain is 5′ of the primer binding site, the primer binding site is 5′ of the spacer domain, and the spacer domain is 5′ of a second portion of the gRNA core domain, and wherein the first and second portions together form the gRNA core domain.

Embodiment 4 The pegRNA of any one of Embodiments 1-2, wherein: a first ligation sequence is 5′ of a portion of the gRNA core domain, the first portion of the gRNA core domain is 5′ of the nucleic acid synthesis template domain, the nucleic acid synthesis template domain is 5′ of the primer binding site, the primer binding site is 5′ of the spacer domain, the spacer domain is 5′ of the second portion of the gRNA core domain, and a second portion of the gRNA core domain is 5′ of a second ligation sequence, and wherein the first and second portions together form the gRNA core domain, and optionally, a portion of the first ligation sequence is complementary to a portion of the second ligation sequence.

Embodiment 5: The pegRNA of Embodiment 3 or 4, further comprising a first linking domain between the primer binding site and the spacer domain.

Embodiment 6: The pegRNA of Embodiment 5, wherein the first linking domain does not form a secondary structure.

Embodiment 7: The pegRNA of Embodiment 5, wherein the first linking domain forms at least one secondary structure, (e.g., a hairpin).

Embodiment 8: The pegRNA of any one of Embodiments 5-7, wherein the first linking domain is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides in length, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 80 nucleotides, at least 55 nucleotides, at least 90 nucleotides, at least 95 nucleotides, at least 100 nucleotides, at least 150 nucleotides, at least 200 nucleotides, or at least 250 nucleotides in length.

Embodiment 9: The pegRNA of any one of Embodiments 3-8, further comprising a second linking domain between the first portion of the gRNA core domain and the second portion of the gRNA core domain.

Embodiment 10: The pegRNA of Embodiment 9, wherein the second linking domain does not form a secondary structure.

Embodiment 11: The pegRNA of Embodiment 9, wherein the second linking domain forms at least one secondary structure, (e.g., a hairpin).

Embodiment 11: The pegRNA of any one of Embodiments 9-11, wherein the second linking domain is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides in length, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 80 nucleotides, at least 55 nucleotides, at least 90 nucleotides, at least 95 nucleotides, at least 100 nucleotides, at least 150 nucleotides, at least 200 nucleotides, or at least 250 nucleotides in length.

Embodiment 12: The pegRNA of any one of Embodiments 1-8, wherein the pegRNA is a RNA:DNA chimera.

Embodiment 13: The pegRNA of any one of Embodiments 1-9, wherein nucleic acid synthesis template domain is a template for an RNA-dependent polymerase (e.g., reverse transcriptase).

Embodiment 14: The pegRNA of any one of Embodiments 1-9, wherein the nucleic acid synthesis template domain is a template for a DNA-dependent polymerase (e.g., DNA polymerase, such as Bsu polymerase or phiDNA polymerase).

Embodiment 15: The pegRNA of any one of Embodiments 1-11, wherein the nucleic acid synthesis template domain and the primer binding site are directly adjacent to each other.

Embodiment 17: The pegRNA of Embodiment 12, wherein the nucleic acid synthesis template domain is positioned 5′ to the primer binding site.

Embodiment 18: The pegRNA of any one of Embodiments 1-13, wherein the nucleic acid synthesis template domain is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides in length, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 80 nucleotides, at least 55 nucleotides, at least 90 nucleotides, at least 95 nucleotides, at least 100 nucleotides, at least 150 nucleotides, at least 200 nucleotides, at least 250 nucleotides, at least 500 nucleotides, at least 750 nucleotides, at least 1 k nucleotides, at least 1.5 k nucleotides, at least 2 k, at least 2 k nucleotides, at least 2.5 k nucleotides, at least 3 k nucleotides, at least 3.5 k nucleotides, at least 4 k nucleotides, at least 4.5 k nucleotides, at least 5 k nucleotides, at least 5.5 k nucleotides, at least 6 k nucleotides, at least 6.5 k nucleotides, at least 7 k nucleotides, at least 7.5 k nucleotides, at least 8 k nucleotides, at least 8.5 k nucleotides, at least 91 k nucleotides, at least 9.5 k nucleotides, or at least 10 k nucleotides in length.

Embodiment 19: The pegRNA of any one of Embodiments 1-13, wherein the nucleic acid synthesis template domain is from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, or from 7 to 17 nucleotides in length.

Embodiment 20: The pegRNA of anyone of Embodiments 1-15, wherein the one or more nucleotide changes comprises insertions of one or more nucleotides, substitutions of one or more nucleotides, deletions of one or more nucleotides, or a combination of any such nucleotide changes, as compared to the double-stranded target DNA sequence.

Embodiment 21: The pegRNA of anyone of Embodiments 1-16, wherein the one or more nucleotide changes comprises a transition selected from the group consisting of: (a) T to C; (b) A to G; (c) C to T; (d) G to A; and (e) A to I.

Embodiment 22: The pegRNA of anyone of Embodiments 1-16, wherein the one or more nucleotide changes comprises a transversion selected from the group consisting of: (a) T to A; (b) T to G; (c) C to G; (d) C to A; (e) A to T; (f) A to C; (g) G to C; (h) G to T; (i) and A to I.

Embodiment 23: The pegRNA of anyone of Embodiments 1-16, wherein the one or more nucleotide changes comprises changing (1) a G:C basepair to a T:A basepair, (2) a G:C basepair to an A:T basepair, (3) a G:C basepair to C:G basepair, (4) a T:A basepair to a G:C basepair, (5) a T:A basepair to an A:T basepair, (6) a T:A basepair to a C:G basepair, (7) a C:G basepair to a G:C basepair, (8) a C:G basepair to a T:A basepair, (9) a C:G basepair to an A:T basepair, (10) an A:T basepair to a T:A basepair, (11) an A:T basepair to a G:C basepair, or (12) an A:T basepair to a C:G basepair.

Embodiment 24: The pegRNA of any one of Embodiments 1-16, wherein the one or more nucleotide changes comprises insertion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides.

Embodiment 25: The pegRNA of anyone of Embodiments 1-16, wherein the one or more nucleotide changes comprises deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides.

Embodiment 26: The pegRNA of any one of Embodiments 1-21, wherein a position of the one or more nucleotides changes is at position +1, +2, +3, +4, +, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, +55, +56, +57, +58, +59, +60, +61, +62, +63, +64, +65, +66, +67, +68, +69, +70, +71, +72, +73, +74, +75, +76, +77, +78, +79, +80, +81, +82, +83, +84, +85, +86, +87, +88, +89, +90, +91, +92, +93, +94, +95, +96, +97, +98, +99, +100, +101, +102, +103, +104, +105, +106, +107, +108, +109, +110, +111, +112, +113, +114, +115, +116, +117, +118, +119, +120, +121, +122, +123, +124, +125, +126, +127, +128, +129, +130, +131, +132, +133, +134, +135, +136, +137, +138, +139, +140, +141, +142, +143, +144, +145, +146, +147, +148, +149, or +150, or more (relative to the downstream position of a nick site).

Embodiment 27: The pegRNA of anyone of Embodiments 1-26, wherein at least a part of the nucleic acid synthesis template domain comprises a sequence substantially complementary to a region downstream of a nick region in a second strand of the double-stranded target nucleic acid.

Embodiment 28: The pegRNA of any one of Embodiments 1-27, wherein the primer binding site is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides in length, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 80 nucleotides, at least 55 nucleotides, at least 90 nucleotides, at least 95 nucleotides, at least 100 nucleotides, at least 150 nucleotides, at least 200 nucleotides, at least 250 nucleotides, at least 275 nucleotides, or at least 300 nucleotides in length

Embodiment 29: The pegRNA of any one of Embodiments 1-27, wherein the primer binding site is from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, from 7 to 17 nucleotides, or from 50 nucleotides to 300 nucleotides in length.

Embodiment 30: The pegRNA of any one of Embodiments 1-29, wherein the primer binding site comprises a sequence having 100% complementarity to a region upstream of the nick site in the second strand of the double-stranded target nucleic acid.

Embodiment 31: The pegRNA of any one of Embodiments 1-30, wherein the spacer domain is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides in length, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 80 nucleotides, at least 55 nucleotides, at least 90 nucleotides, at least 95 nucleotides, at least 100 nucleotides, at least 150 nucleotides, at least 200 nucleotides, or at least 250 nucleotides in length

Embodiment 32: The pegRNA of any one of Embodiments 1-30, wherein the spacer domain is from 3 to 50 nucleotides, from 4 to 45 nucleotides, from 6 to 40 nucleotides, from 7 to 35 nucleotides, from 8 to 30 nucleotides, from 9 to 25 nucleotides, from 10 to 20 nucleotides, from 10 to 16 nucleotides, from 12 to 17 nucleotides, from 8 to 15 nucleotides, from 3 to 20 nucleotides, from 7 to 17 nucleotides in length, or from 20 nucleotide to 200 nucleotides.

Embodiment 33: The pegRNA of any one of Embodiments 1-31, wherein the spacer domain comprises a sequence having 100% complementarity to the first strand of the double-stranded target nucleic acid, or the spacer domain comprises a sequence having one or more (e.g., 1, 2, 3, 4, or 5) mismatches with the first strand of the double-stranded target nucleic acid.

Embodiment 34: The pegRNA of any one of Embodiments 1-33, wherein the gRNA core domain comprises one or more secondary structures.

Embodiment 35: The pegRNA of any one of Embodiments 1-34, wherein the gRNA core domain comprises at least one (e.g., two, three or more) hairpins.

Embodiment 36: The pegRNA of any one of Embodiments 1-35, wherein the gRNA core domain comprises a nucleotide sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to a sequence selected from the group consisting of:

(SEQ ID NO: 604)
GTTTCAGAGCTATGCTGGAAACAGCATAGCAAGTTGAAATAAGGCTAGT
CCGTTATCAACTTGAAAAAGTGGCACCGAGTCGGTGC;
and
(SEQ ID NO: 572)
GTTTTAGAGCTAGAAATAGCAAGTTAAAATAAGGCTAGTCCGTTATCAA
CTTGAAAAAGTGGGACCGAGTCGGTCC.

Embodiment 37: The pegRNA of any one of Embodiments 1-36, wherein the pegRNA does not comprise an RNA-binding protein recruitment domain.

Embodiment 38: The pegRNA of any one of Embodiments 1-36, further comprising an RNA-binding protein recruitment domain.

Embodiment 39: The pegRNA of Embodiment 38, wherein the RNA-binding protein recruitment domain is positioned 3′ to the primer binding site.

Embodiment 40: The pegRNA of Embodiment 387, wherein the RNA-binding protein recruitment domain is positioned 5′ to the primer binding site.

Embodiment 41: The pegRNA of Embodiment 38, wherein the RNA-binding protein recruitment domain is positioned 3′ to the spacer.

Embodiment 42: The pegRNA of Embodiment 38, wherein the RNA-binding protein recruitment domain is positioned 5′ to the spacer.

Embodiment 43: The pegRNA of any one of Embodiments 37-42, wherein the RNA-binding protein recruitment domain is an aptamer sequence.

Embodiment 44: The pegRNA of Embodiment 43, wherein the aptamer sequence is a MS2 aptamer sequence.

Embodiment 45: The pegRNA of any one of Embodiments 1-44, wherein the pegRNA is circularized.

Embodiment 46: The pegRNA of any one of Embodiments 1-44, wherein the pegRNA comprises a first portion of the gRNA core domain at one of the 5′-end or the 3′-end, and a second portion of the gRNA core domain at the other of the 5′-end or the 3′-end, and wherein the first and second portions together form the gRNA core domain.

Embodiment 47: The pegRNA of any one of Embodiments 1-44, wherein the pegRNA comprises a first ribozyme and a first ligation sequence positioned 3′ to the first ribozyme at 5′-end, and a second ribozyme and a second ligation sequence positioned 3′ to the second ribozyme at the 3′-end, and wherein a portion of the first ligation sequence is complementary to a portion of the first ribozyme and a portion of the second ligation sequence is complementary to a portion of the second ribozyme, wherein a portion of the first ligation sequence is complementary to a portion of the second ligation sequence; and wherein the portion of the first ligation sequence complementary to the portion of the first ribozyme is complementary to the portion of the second ligation sequence complementary to the portion of the second ribozyme.

Embodiment 48: The pegRNA of Embodiment 47, wherein each of the first ribozyme and the second ribozyme comprises a sequence that may be cleaved to produce a 5′-OH end and a 2′,3′-cyclic phosphate end.

Embodiment 49: The pegRNA of Embodiment 47 or 48, wherein each of the first and the second ribozyme is independently selected from the group consisting of Hammerhead, Hairpin, Hepatitis Delta Virus (“HDV”), Varkud Satellite (“VS”), Vg1, glucosamine-6-phosphate synthase (“glmS”), Twister, Twister Sister, Hatchet, Pistol ribozymes, engineered synthetic ribozymes, or derivatives thereof.

Embodiment 50: The pegRNA of any one of Embodiments 47-49, wherein each of the first and the second ribozyme is, independently, a split ribozyme or ligand-activated ribozyme derivative.

Embodiment 51: The pegRNA of any one of Embodiments 46-50, wherein the first ribozyme is a P3 Twister ribozyme and the second ribozyme is a P1 Twister ribozyme.

Embodiment 52: The pegRNA of any one of Embodiments 47-51, wherein each of the first ligation sequence and the second ligation sequence are substrates for an RNA ligase.

Embodiment 53: The pegRNA of any one of Embodiments 47-52, wherein each of the first ligation sequence and the second ligation sequence comprise a portion of a tRNA exon sequence or derivative thereof.

Embodiment 54: The pegRNA of Embodiment 53, wherein the RNA ligase is RtcB.

Embodiment 55: The pegRNA of any one of Embodiments 1-54, wherein the nucleic acid programmable DNA binding protein has nickase activity.

Embodiment 56: The pegRNA of any one of Embodiments 1-55, wherein the nucleic acid programmable DNA binding protein is an RNA guided DNA-binding protein, optionally the nucleic acid programmable DNA binding protein is a CRISPR Cas enzyme, an Argonaute protein, an obligate mobile element guided activity (OMEGA) enzyme, a RuVC nucleases, or a homolog, ortholog or variant thereof.

Embodiment 57: The pegRNA of any one of Embodiments 1-56, wherein the nucleic acid programmable DNA binding protein is selected from the group consisting of: Cas9 (also known as CsnI and CsxI2), Cas1, Cas100, Cas12a (Cpf1), Cas12b, Cas12b1 (C2c1), Cas12b2, Cas12c (C2c3), Cas12d (CasY), Cas12e (CasX), Cas13a (C2c2), Cas13b (C2c6), Cas13c (C2c7), Cas1B, Cas2, Cas3, Cas4, Cas5, Cas6, Cas7, Cas8, CasI, CasIB, CasIO, Cmr1, Cmr3, Cmr4, Cmr5, Cmr6, Cpf1, Csa5, Csa5, CsaX, Csb1, Csb2, Csb3, Csc1, Csc2, C2c5, C2c8, C2c9, C2c10, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, Ago1, Ago2, Ago3, Ago4, Fz TnpB, Fz, IS110 family recombinases (e.g., IS621, and homologs, orthologs and variants thereof.

Embodiment 58: The pegRNA of any one of Embodiments 1-57, wherein the nucleic acid programmable DNA binding protein is a Cas9.

Embodiment 59: The pegRNA of any one of Embodiments 1-58, wherein the nucleic acid programmable DNA binding protein is a mutated Cas9, optionally the mutated Cas9 comprises a dead HNH domain or a dead RuVC domain, and/or the mutated Cas9 is shorter than a wildtype Cas9.

Embodiment 60: The pegRNA of any one of Embodiments 1-59, wherein the nucleic acid programmable DNA binding protein is Cas9 nickase (nCas9).

Embodiment 61: The pegRNA of any one of Embodiments 1-60, wherein the nucleic acid modifying enzyme is a polymerase, an RNA deaminase, an RNA methylase, an RNA demethylase, a retrotransposon or an integrase fused with a polymerase.

Embodiment 62: The pegRNA of any one of Embodiments 1-61, wherein the nucleic acid modifying enzyme is a polymerase.

Embodiment 63: The pegRNA of Embodiment 62, wherein the polymerase is an RNA-dependent polymerase (e.g., reverse transcriptase).

Embodiment 64: The pegRNA of Embodiment 63, wherein the reverse transcriptase is a reverse transcriptase from a retrovirus or a retrotransposon.

Embodiment 65: The pegRNA of Embodiment 63, wherein the reverse transcriptase is a Moloney-Murine Leukemia Virus reverse transcriptase (M-MLV RT) or a variant of M-MLV RT.

Embodiment 66: The pegRNA of Embodiment 62, wherein the polymerase is a DNA-dependent polymerase (e.g., DNA polymerase, such as Bsu polymerase or phiDNA polymerase).

Embodiment 67: The pegRNA of any one of Embodiments 1-66, wherein the nucleic acid modifying enzyme lacks nuclease activity.

Embodiment 68: The pegRNA of any one of Embodiments 1-67, wherein the pegRNA comprises at least one nucleic acid modification.

Embodiment 69: The pegRNA of any one of Embodiments 1-67, wherein the pegRNA comprises at least one nucleic acid modification selected from the group consisting of modified internucleoside linkages, modified nucleobases, modified sugars, and any combinations thereof.

Embodiment 70: A nucleic acid encoding a pegRNA of any one of Embodiments 1-69.

Embodiment 71: A prime editing system, comprising: (a) a pegRNA of any one of Embodiments 1-68 or a nucleic acid encoding same; (b) a nucleic acid programmable DNA binding protein (napDNAbp); and (c) a nucleic acid modifying enzyme or a nucleic acid encoding same.

Embodiment 72: The prime editing system of Embodiment 71, wherein the nucleic acid programmable DNA binding protein is not attached or tethered to the nucleic acid modifying enzyme.

Embodiment 73: The prime editing system of Embodiment 71, wherein the nucleic acid programmable DNA binding protein is attached or tethered to the nucleic acid modifying enzyme.

Embodiment 74: The prime editing system of Embodiment 73, wherein the nucleic acid programmable DNA binding protein and the nucleic acid modifying enzyme are comprised in a fusion protein.

Embodiment 75: The prime editing system of any one of Embodiments 71-74, wherein the pegRNA does not comprise an RNA-binding protein recruitment domain (e.g., a MS2 aptamer sequence), and optionally the pegRNA is circularized.

Embodiment 76: The prime editing system of any one of Embodiments 71-75, wherein the nucleic acid programmable DNA binding protein has nickase activity.

Embodiment 77: The prime editing system of any one of Embodiments 71-76, wherein the nucleic acid programmable DNA binding protein is an RNA guided DNA-binding protein, optionally the nucleic acid programmable DNA binding protein is a CRISPR Cas enzyme, an Argonaute protein, an obligate mobile element guided activity (OMEGA) enzyme, a RuVC nucleases, or a homolog, ortholog or variant thereof.

Embodiment 78: The prime editing system of any one of Embodiments 71-77, wherein the nucleic acid programmable DNA binding protein is selected from the group consisting of: Cas9 (also known as CsnI and CsxI2), Cas1, Cas100, Cas12a (Cpf1), Cas12b, Cas12b1 (C2c1), Cas12b2, Cas12c (C2c3), Cas12d (CasY), Cas12e (CasX), Cas13a (C2c2), Cas13b (C2c6), Cas13c (C2c7), Cas1B, Cas2, Cas3, Cas4, Cas5, Cas6, Cas7, Cas8, CasI, CasIB, CasIO, Cmr1, Cmr3, Cmr4, Cmr5, Cmr6, Cpf1, Csa5, Csa5, CsaX, Csb1, Csb2, Csb3, Csc1, Csc2, C2c5, C2c8, C2c9, C2c10, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, Ago1, Ago2, Ago3, Ago4, Fz TnpB, Fz, IS110 family recombinases (e.g., IS621, and homologs, orthologs and variants thereof.

Embodiment 79: The prime editing system of any one of Embodiments 71-78, wherein the nucleic acid programmable DNA binding protein is a Cas9.

Embodiment 80: The prime editing system of any one of Embodiments 71-79, wherein the nucleic acid programmable DNA binding protein is a mutated Cas9, optionally the mutated Cas9 comprises a dead HNH domain or a dead RuVC domain, and/or the mutated Cas9 is shorter than a wildtype Cas9.

Embodiment 81: The prime editing system of any one of Embodiments 71-80, wherein the nucleic acid programmable DNA binding protein is Cas9 nickase (nCas9).

Embodiment 82: The prime editing system of any one of Embodiments 71-81, wherein the nucleic acid modifying enzyme is a polymerase, an RNA deaminase, an RNA methylase, an RNA demethylase, a retrotransposon or an integrase fused with a polymerase.

Embodiment 83: The prime editing system of any one of Embodiments 71-82, wherein the nucleic acid modifying enzyme is a polymerase.

Embodiment 84: The prime editing system of Embodiment 83, wherein the polymerase is an RNA-dependent polymerase (e.g., reverse transcriptase).

Embodiment 85: The pegRNA of Embodiment 84, wherein the reverse transcriptase is a reverse transcriptase from a retrovirus or a retrotransposon.

Embodiment 86: The pegRNA of Embodiment 84, wherein the reverse transcriptase is a Moloney-Murine Leukemia Virus reverse transcriptase (M-MLV RT) or a variant of M-MLV RT.

Embodiment 87: The prime editing system of Embodiment 83, wherein the polymerase is a DNA-dependent polymerase (e.g., DNA polymerase, such as Bsu polymerase or phiDNA polymerase).

Embodiment 88: The prime editing system of Embodiments 71-87, wherein the nucleic acid modifying enzyme lacks nuclease activity.

Embodiment 89: A composition comprising a pegRNA of any one of Embodiments 1-67 or a nucleic acid encoding same.

Embodiment 90: The composition of Embodiment 89, further comprising a nucleic acid programmable DNA binding protein, or a nucleic acid encoding same.

Embodiment 91: The composition of Embodiment 90, wherein the nucleic acid programmable DNA binding protein has nickase activity.

Embodiment 92: The composition of any one of Embodiments 90-91, wherein the nucleic acid programmable DNA binding protein is an RNA guided DNA-binding protein, optionally the nucleic acid programmable DNA binding protein is a CRISPR Cas enzyme, an Argonaute protein, an obligate mobile element guided activity (OMEGA) enzyme, a RuVC nucleases, or a homolog, ortholog or variant thereof.

Embodiment 93: The composition of any one of Embodiments 90-92, wherein the nucleic acid programmable DNA binding protein is selected from the group consisting of: Cas9 (also known as CsnI and CsxI2), Cas1, Cas100, Cas12a (Cpf1), Cas12b, Cas12b1 (C2c1), Cas12b2, Cas12c (C2c3), Cas12d (CasY), Cas12e (CasX), Cas13a (C2c2), Cas13b (C2c6), Cas13c (C2c7), Cas1B, Cas2, Cas3, Cas4, Cas5, Cas6, Cas7, Cas8, CasI, CasIB, CasIO, Cmr1, Cmr3, Cmr4, Cmr5, Cmr6, Cpf1, Csa5, Csa5, CsaX, Csb1, Csb2, Csb3, Csc1, Csc2, C2c5, C2c8, C2c9, C2c10, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, Ago1, Ago2, Ago3, Ago4, Fz TnpB, Fz, IS110 family recombinases (e.g., IS621, and homologs, orthologs and variants thereof.

Embodiment 94: The composition of any one of Embodiments 90-93, wherein the nucleic acid programmable DNA binding protein is a Cas9.

Embodiment 95: The composition of any one of Embodiments 90-94, wherein the nucleic acid programmable DNA binding protein is a mutated Cas9, optionally the mutated Cas9 comprises a dead HNH domain or a dead RuVC domain, and/or the mutated Cas9 is shorter than a wildtype Cas9.

Embodiment 96: The composition of any one of Embodiments 90-95, wherein the nucleic acid programmable DNA binding protein is Cas9 nickase (nCas9).

Embodiment 97: The composition of any one of Embodiments 89-96, further comprising a nucleic acid modifying enzyme or a nucleic acid encoding same.

Embodiment 981: The composition of Embodiment 97, wherein the nucleic acid modifying enzyme is a polymerase, an RNA deaminase, an RNA methylase, an RNA demethylase, or a transposon.

Embodiment 99: The composition of any one of Embodiments 96-98, wherein the nucleic acid modifying enzyme is a polymerase.

Embodiment 10: The composition of Embodiment 99, wherein the polymerase is an RNA-dependent polymerase (e.g., reverse transcriptase).

Embodiment 101: The composition of Embodiment 100, wherein the reverse transcriptase is a reverse transcriptase from a retrovirus or a retrotransposon.

Embodiment 102: The composition of Embodiment 100, wherein the reverse transcriptase is a Moloney-Murine Leukemia Virus reverse transcriptase (M-MLV RT) or a variant of M-MLV RT.

Embodiment 103: The composition of Embodiment 98, wherein the polymerase is a DNA-dependent polymerase (e.g., DNA polymerase, such as Bsu polymerase or phiDNA polymerase).

Embodiment 104: The composition of any one of Embodiments 97-104, wherein the nucleic acid modifying enzyme lacks nuclease activity.

Embodiment 105: The composition of any one of Embodiments 89-105, further comprising a pharmaceutically acceptable carrier or excipient.

Embodiment 106: The composition of any one of Embodiments 89-105, further comprising a nucleic acid delivery system, e.g., virus like particle such as engineered virus like particle.

Embodiment 107: A kit comprising a pegRNA of any one of Embodiments 1-67.

Embodiment 108: The kit of Embodiment 107, further comprising a nucleic acid programmable DNA binding protein, or a nucleic acid encoding same.

Embodiment 109: The kit of Embodiment 108, wherein the nucleic acid programmable DNA binding protein has nickase activity.

Embodiment 110: The kit of any one of Embodiments 108-109, wherein the nucleic acid programmable DNA binding protein is an RNA guided DNA-binding protein, optionally the nucleic acid programmable DNA binding protein is a CRISPR Cas enzyme.

Embodiment 111: The kit of any one of Embodiments 108-110, wherein the nucleic acid programmable DNA binding protein is selected from the group consisting of: Cas9 (also known as CsnI and CsxI2), Cas1, Cas100, Cas12a (Cpf1), Cas12b, Cas12b1 (C2c1), Cas12b2, Cas12c (C2c3), Cas12d (CasY), Cas12e (CasX), Cas13a (C2c2), Cas13b (C2c6), Cas13c (C2c7), Cas1B, Cas2, Cas3, Cas4, Cas5, Cas6, Cas7, Cas8, CasI, CasIB, CasIO, Cmr1, Cmr3, Cmr4, Cmr5, Cmr6, Cpf1, Csa5, Csa5, CsaX, Csb1, Csb2, Csb3, Csc1, Csc2, C2c5, C2c8, C2c9, C2c10, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, an Argonaute protein, an obligate mobile element guided activity (OMEGA) enzyme, a RuVC nucleases, or a homolog, ortholog or variant thereof.

Embodiment 112: The kit of any one of Embodiments 108-111, wherein the nucleic acid programmable DNA binding protein is a Cas9.

Embodiment 113: The kit of any one of Embodiments 108-112, wherein the nucleic acid programmable DNA binding protein is a mutated Cas9, optionally the mutated Cas9 comprises a dead HNH domain or a dead RuVC domain, and/or the mutated Cas9 is shorter than a wildtype Cas9.

Embodiment 114: The kit of any one of Embodiments 108-113, wherein the nucleic acid programmable DNA binding protein is Cas9 nickase (nCas9).

Embodiment 115: The kit of any one of Embodiments 107-114, further comprising a nucleic acid modifying enzyme or a nucleic acid encoding same.

Embodiment 116: The kit of Embodiment 115, wherein the nucleic acid modifying enzyme is a polymerase, an RNA deaminase, an RNA methylase, an RNA demethylase, a retrotransposon or an integrase fused with a polymerase.

Embodiment 117: The kit of any one of Embodiments 115-116, wherein the nucleic acid modifying enzyme is a polymerase.

Embodiment 118: The kit of Embodiment 117, wherein the polymerase is an RNA-dependent polymerase (e.g., reverse transcriptase).

Embodiment 119: The kit of Embodiment 118, wherein the reverse transcriptase is a reverse transcriptase from a retrovirus or a retrotransposon.

Embodiment 120: The kit of Embodiment 118, wherein the reverse transcriptase is a Moloney-Murine Leukemia Virus reverse transcriptase (M-MLV RT) or a variant of M-MLV RT.

Embodiment 121: The kit of Embodiment 116, wherein the polymerase is a DNA-dependent polymerase (e.g., DNA polymerase, such as Bsu polymerase or phiDNA polymerase).

Embodiment 122: The kit of any one of Embodiments 115-121, wherein the nucleic acid modifying enzyme lacks nuclease activity.

Embodiment 123: The kit of any one of Embodiments 107-122, further comprising a pharmaceutically acceptable carrier or excipient.

Embodiment 124: The kit of any one of Embodiments 107-122, further comprising a nucleic acid delivery system.

Embodiment 125: A cell comprising a pegRNA of any one of Embodiments 1-67.

Embodiment 126: The cell of Embodiment 125, wherein the cell is a mammalian cell.

Embodiment 127: The cell of Embodiment 125 or 126, wherein the cell is a human cell.

Embodiment 128: The cell of anyone of Embodiments 125-127, wherein the cell is a mismatch repair (MMR) deficient cell.

Embodiment 129: The cell of any one of Embodiments 125-127, wherein the cell is a mismatch repair (MMR) competent cell.

Embodiment 130: The cell of any one of Embodiments 125-127, wherein the cell is selected from the group consisting of hematopoietic stem cells, T cells, liver cells (e.g., hepatocytes, pancreatic islet beta cells, and lung epithelial cells.

Embodiment 131: The cell of any one of Embodiments 125-130, further comprising a nucleic acid programmable DNA binding protein, or a nucleic acid encoding same.

Embodiment 132: The cell of Embodiment 131, wherein the nucleic acid programmable DNA binding protein has nickase activity.

Embodiment 133: The cell of any one of Embodiments 131-132, wherein the nucleic acid programmable DNA binding protein is an RNA guided DNA-binding protein, optionally the nucleic acid programmable DNA binding protein is a CRISPR Cas enzyme, an Argonaute protein, an obligate mobile element guided activity (OMEGA) enzyme, a RuVC nucleases, or a homolog, ortholog or variant thereof.

Embodiment 134: The cell of any one of Embodiments 131-133, wherein the nucleic acid programmable DNA binding protein is selected from the group consisting of: Cas9 (also known as CsnI and CsxI2), Cas1, Cas100, Cas12a (Cpf1), Cas12b, Cas12b1 (C2c1), Cas12b2, Cas12c (C2c3), Cas12d (CasY), Cas12e (CasX), Cas13a (C2c2), Cas13b (C2c6), Cas13c (C2c7), Cas1B, Cas2, Cas3, Cas4, Cas5, Cas6, Cas7, Cas8, CasI, CasIB, CasIO, Cmr1, Cmr3, Cmr4, Cmr5, Cmr6, Cpf1, Csa5, Csa5, CsaX, Csb1, Csb2, Csb3, Csc1, Csc2, C2c5, C2c8, C2c9, C2c10, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, Ago1, Ago2, Ago3, Ago4, Fz TnpB, Fz, IS110 family recombinases (e.g., IS621, and homologs, orthologs and variants thereof.

Embodiment 135: The cell of any one of Embodiments 131-134, wherein the nucleic acid programmable DNA binding protein is a Cas9.

Embodiment 136: The cell of any one of Embodiments 131-135, wherein the nucleic acid programmable DNA binding protein is a mutated Cas9, optionally the mutated Cas9 comprises a dead HNH domain or a dead RuVC domain, and/or the mutated Cas9 is shorter than a wildtype Cas9.

Embodiment 137: The cell of any one of Embodiments 131-136, wherein the nucleic acid programmable DNA binding protein is Cas9 nickase (nCas9).

Embodiment 138: The cell of any one of Embodiments 125-137, further comprising a nucleic acid modifying enzyme or a nucleic acid encoding same.

Embodiment 139: The cell of Embodiment 138, wherein the nucleic acid modifying enzyme is a polymerase, an RNA deaminase, an RNA methylase, or an RNA demethylase.

Embodiment 140: The cell of any one of Embodiments 138-139, wherein the nucleic acid modifying enzyme is a polymerase.

Embodiment 141: The cell of Embodiment 140, wherein the polymerase is an RNA-dependent polymerase (e.g., reverse transcriptase).

Embodiment 142: The cell of Embodiment 141, wherein the reverse transcriptase is a reverse transcriptase from a retrovirus or a retrotransposon.

Embodiment 143: The cell of Embodiment 142, wherein the reverse transcriptase is a Moloney-Murine Leukemia Virus reverse transcriptase (M-MLV RT) or a variant of M-MLV RT.

Embodiment 144: The cell of Embodiment 140, wherein the polymerase is a DNA-dependent polymerase (e.g., DNA polymerase, such as Bsu polymerase or phiDNA polymerase).

Embodiment 145: The cell of any one of Embodiments 138-144, wherein the nucleic acid modifying enzyme lacks nuclease activity.

Embodiment 146: The cell of any one of Embodiments 125-146, wherein the cell is in vitro.

Embodiment 147: The cell of any one of Embodiments 125-146, wherein the cell is ex vivo.

Embodiment 148: The cell of anyone of Embodiments 125-146, wherein the cell is in vivo.

Embodiment 149: The cell of any one of Embodiments 125-146, wherein the cell is a modified cell.

Embodiment 150: A method of introducing one or more changes in the nucleotide sequence of a target nucleic acid, the method comprising contacting a double-stranded target nucleic acid (e.g., DNA) with a prime editing system of any one of Embodiments 71-81.

Embodiment 151: The method of Embodiment 150, wherein the target nucleic acid is in a cell.

Embodiment 152: The method of Embodiment 151, where the cell is a mammalian cell.

Embodiment 153: The method of Embodiment 151, wherein the cell is human cell.

Embodiment 154: The method of any one of Embodiments 150-153, wherein the cell is a mismatch repair (MMR) deficient cell.

Embodiment 155: The method of any one of Embodiments 150-153, wherein the cell is a mismatch repair (MMR) competent cell.

Embodiment 156: The method of any one of Embodiments 150-153, wherein the cell is selected from the group consisting of hematopoietic stem cell, T cells, liver cells (e.g., hepatocytes), pancreatic islet beta cells, and lung epithelial cells.

Embodiment 157: The method of anyone of Embodiments 150-156, wherein the one or more changes in the nucleotide sequence comprises a correction to a disease-associated gene.

Embodiment 158: The method of Embodiment 157,where the disease associated gene is associated with disorder selected from the group consisting of: Phenylketonuria; Hyperphenylalaninemia; Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; a trinucleotide repeat disorder; a prion disease; Tay-Sachs Disease; heart disease; high blood pressure; Alzheimer's disease; arthritis; diabetes; cancer; and obesity

Embodiment 159: The method of any one of Embodiments 150-158, wherein the one or more nucleotide changes comprises insertions of one or more nucleotides, substitutions of one or more nucleotides, deletions of one or more nucleotides, or a combination of any such nucleotide changes, as compared to the double-stranded target DNA sequence.

Embodiment 160: The method of any one of Embodiments 150-159, wherein the one or more nucleotide changes comprises a transition selected from the group consisting of: (a) T to C; (b) A to G; (c) C to T; (d) G to A; and (e) A to I.

Embodiment 161: The method of any one of Embodiments 150-159, wherein the one or more nucleotide changes comprises a transversion selected from the group consisting of: (a) T to A; (b) T to G; (c) C to G; (d) C to A; (e) A to T; (f) A to C; (g) G to C; (h) G to T; (i) and A to I.

Embodiment 162: The method of any one of Embodiments 150-159, wherein the one or more nucleotide changes comprises changing (1) a G:C basepair to a T:A basepair, (2) a G:C basepair to an A:T basepair, (3) a G:C basepair to C:G basepair, (4) a T:A basepair to a G:C basepair, (5) a T:A basepair to an A:T basepair, (6) a T:A basepair to a C:G basepair, (7) a C:G basepair to a G:C basepair, (8) a C:G basepair to a T:A basepair, (9) a C:G basepair to an A:T basepair, (10) an A:T basepair to a T:A basepair, (11) an A:T basepair to a G:C basepair, or (12) an A:T basepair to a C:G basepair.

Embodiment 163: The method of any one of Embodiments 150-159, wherein the one or more nucleotide changes comprises insertion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides.

Embodiment 164: The method of any one of Embodiments 150-159, wherein the one or more nucleotide changes comprises deletion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 nucleotides.

Embodiment 165: The method of any one of Embodiments 150-164, wherein a position of the one or more nucleotides changes is at position+1, +2, +3, +4, +, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, +55, +56, +57, +58, +59, +60, +61, +62, +63, +64, +65, +66, +67, +68, +69, +70, +71, +72, +73, +74, +75, +76, +77, +78, +79, +80, +81, +82, +83, +84, +85, +86, +87, +88, +89, +90, +91, +92, +93, +94, +95, +96, +97, +98, +99, +100, +101, +102, +103, +104, +105, +106, +107, +108, +109, +110, +111, +112, +113, +114, +115, +116, +117, +118, +119, +120, +121, +122, +123, +124, +125, +126, +127, +128, +129, +130, +131, +132, +133, +134, +135, +136, +137, +138, +139, +140, +141, +142, +143, +144, +145, +146, +147, +148, +149, or +150, or more (relative to the downstream position of a nick site).

Embodiment 166: The method of any one of Embodiments 150-165, wherein the method is a therapeutic gene editing method.

Embodiment 167: The method of Embodiments 166, wherein the method of therapeutic genome editing comprising administering to a target cell selected from the group consisting of: (a) hematopoietic stem cells; (b) T cells; (c) liver cells (hepatocytes); (d) pancreatic islet beta cells; (e) lung epithelial cells.

Embodiment 168: The method of Embodiment 167, wherein the genome-editing composition is selected from the group consisting of: (a) an autologous, ex vivo CRISPR/Cas9 gene-edited hematopoietic stem cell therapy for the treatment of sickle cell disease or β-thalassemia; (b) an allogeneic CRISPR/Cas9 gene-edited CAR T cell therapy targeting CD19+ malignancies and autoimmune diseases; (c) an allogeneic CRISPR/Cas9 gene-edited CAR T cell therapy targeting CD70 for the treatment of solid tumors and hematological malignancies; (d) an in vivo gene-editing therapy utilizing lipid nanoparticle (LNP) delivery to target ANGPTL3 for cardiovascular disease; (e) an in vivo gene-editing therapy utilizing LNP delivery to target Lp(a) for cardiovascular disease; (f) an in vivo gene-editing therapy utilizing LNP delivery to target hepatic angiotensinogen (AGT) for refractory hypertension; (g) an in vivo gene-editing therapy utilizing LNP delivery to target ALAS1 for acute hepatic porphyria (AHP); (h) an allogeneic, gene-edited, immune-evasive, stem cell-derived beta-cell replacement therapy for Type 1 diabetes mellitus; (i) an ex vivo base editing therapy for sickle cell disease to induce fetal hemoglobin expression; (j) a multiplex base edited anti-CD7 CAR-T cell therapy for the treatment of relapsed and refractory T-cell acute lymphoblastic leukemia and T-cell lymphoblastic lymphoma; (k) a liver-targeting LNP-formulated base editing therapy for glycogen storage disease type 1a; (l) a liver-targeting LNP-formulated base editing therapy for Alpha-1 antitrypsin deficiency; (m) an ex vivo autologous hematopoietic stem cell therapy for the treatment of p47 \hat{ }phox Chronic Granulomatous Disease; (n) an ex vivo hematopoietic stem cell therapy utilizing a prime editing approach for X-linked Chronic Granulomatous Disease; (o) an in vivo prime editing therapy targeting ATP7B for the treatment of Wilson's Disease; and (p) an in vivo prime editing therapy targeting mutations associated with cystic fibrosis.

### Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Other than in the operating examples, or where otherwise indicated, all numbers expressing quantities of ingredients or reaction conditions used herein should be understood as modified in all instances by the term “about.” The term “about” when used in connection with percentages can mean±1%. In some embodiments of the various aspects described herein, the term “about” when used in connection with percentages can mean±5%. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The abbreviation, “e.g.” is derived from the Latin exempli gratia, and is used herein to indicate a non-limiting example. Thus, the abbreviation “e.g.” is synonymous with the term “for example.”

As used herein, “rotated pegRNA”, abbreviated rpegRNA, refers to a linear circularly permutated version of the pegRNA.

As used herein, the terms “identity”, “identical” and the like refer to the sequence similarity between two polymeric molecules, e.g., between two nucleic acid molecules or between two polypeptide molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250). Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10. The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence. For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100. Preferably sequence identity is assessed over the length of the shorter of the two sequences being compared.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

As used herein, the term “substantially complementary”, with respect to a nucleotide sequence in relation to a reference nucleotide sequence means a nucleotide sequence having a percentage of identity between the substantially complementary nucleotide sequence and the exact complementary sequence of said reference of at least at least 80%, e.g., at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% (i.e., exactly complementary). Preferably complementarity is assessed over the length of the shorter of the two sequences being compared.

The term “operably linked”, “operably connected” or equivalent expressions as used herein refer to the arrangement of various nucleic acid elements relative to each such that the elements are functionally connected and are able to interact with each other in the manner intended. Such elements may include, without limitation, a promoter, an enhancer and/or a regulatory element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements. As understood by the skilled person, operably linked implies functional activity, and is not necessarily related to a natural positional link. Indeed, when used in a vector, cis-regulatory elements will typically be located immediately upstream of the promoter (although this is generally the case, it should definitely not be interpreted as a limitation or exclusion of positions within the vector, but this needs not be the case in vivo, e.g., a regulatory element sequence naturally occurring downstream of a gene whose transcription it affects is able to function in the same way when located upstream of the promoter. Hence, in some embodiments, the regulatory or enhancing effect of the regulatory element is position-independent.

The term “template for an RNA-dependent polymerase” as used herein refers to a ribonucleic acid (RNA) sequence that is utilized as a substrate for a RNA-dependent polymerase, such as a reverse transcriptase.

The term “template for an DNA-dependent polymerase” as used herein refers to a deoxyribonucleic acid (DNA) sequence that is utilized as a substrate for a DNA polymerase

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of treatme. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the treatment.

The terms “wild-type” or “wt” or “WT” or “native” as used herein is meant an amino acid sequence or a nucleotide sequence that is found in nature, including allelic variations. A wild-type protein, polypeptide, polynucleotide, DNA, RNA, and the like has an amino acid sequence or a nucleotide sequence that has not been intentionally modified.

In the various embodiments described herein, it is further contemplated that variants (naturally occurring or otherwise), alleles, homologs, conservatively modified variants, and/or conservative substitution variants of any of the particular proteins, polypeptides, enzymes, nucleic acids and polynucleotides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the assays described herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example.

A variant DNA or amino acid sequence can be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “statistically significant” or “significantly” refers to statistical significance and generally means a two standard deviation (2SD) or greater difference.

As used herein, “secondary structure” refers to the structure of a nucleic acid as a function of the basepairing interactions within a single nucleic acid strand or more than one strands. Exemplary secondary structures include, but are not limited to hairpins, stem-loops, pseudoknots, bulges, internal loops, external loops, R-loops, multiloops, kissing hairpins, and multibranched loops (or junctions).

As used herein, “stem-loop structure”, or “stem-loop element” refers to a polynucleotide having a secondary structure that includes a region of nucleotides known or predicted to form a double-stranded region (“stem element”) joined on one side to a region of predominantly single-stranded nucleotides (“loop element”). The terms “hairpin structure” or “hairpin loop” are also used herein to refer to a stem-loop structure. Such structures are well known in the art. Base pairing can be accurate. However, as known in the art, stem elements do not require accurate base pairing. Thus, the stem element may comprise one or more base mismatches or unpaired bases.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, at least 3, at least 4, at least 5, at least 6, at least 7, at least 8, at least 9, at least 10, at least 20, at least 23 or more unpaired nucleotides.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Other terms are defined herein within the description of the various aspects of the disclosure.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1—Engineered Circularized pegRNAs Provide Enhanced Prime Editing

Genomic modification technologies have a wide range of therapeutic and agricultural applications. Prime editors (PEs) are modular molecular machines that can engineer directed base substitution, deletion, or insertion modifications within the genome of the mammalian and plant cells. Prime editor guide RNAs (pegRNAs) direct the PE molecular machinery for the synthesis of the desired genomic alteration. The design and stability of pegRNAs play essential roles in defining PE performance and require multiple rounds of iterative screening. Nevertheless, PEs function less efficiently in Mismatch Repair (MMR) competent cells that account for most of the therapeutically relevant target cell types. Here, the data presented herein demonstrate that engineered circular pegRNAs (cpegRNAs) provide greater than 30-fold increase in PE efficiency in MMR-competent human cells compared to linear pegRNAs. The data further show that cpegRNAs improve the performance of split PE molecular machinery that consists of nCas9 and Mul-V reverse-transcriptase (RT) domains. These results represent a powerful new strategy for prime editing in hard-to-edit MMR-competent cells which can have implications for both ex vivo and in vivo therapeutic applications.

The ability to synthesize and rearrange genomic DNA in the living cells is important for the future of therapeutics and sustainability. Prime editors (PEs) are modular molecular machines that enable targeted genomic synthesis within plants¹, mammalian cells² and animals³. Prime editing can correct for ˜90% of the known human disease variants with minimal bystander editing². Unlike the CRISPR-dependent Homology Directed Repair (HDR) platforms, the PE system does not generate double-strand DNA breaks (DDBs) that are prone to undesired insertions and deletions (Indel) at the target site⁴⁵. The lack of DDBs for prime editing facilitates homogenous alterations at the genomic modification site, which improves the safety of the platform for therapeutic applications.

The PE molecular machinery consists of a fusion between the Cas9 nickase (nCas9) enzyme and a reverse transcriptase (RT) domain from the Molony murine leukemia virus (M-MLV). The initial generation of PEs (PE1-PE2) only requires a prime editing guide RNA (pegRNA) to perform genomic alterations². The pegRNA is composed of a 5′ spacer domain followed by the single guide RNA (sgRNA) scaffold and a 3′ extension composed of the Reverse Transcriptase Template (RTT) and Primer Binding Sequence (PBS) domains. PE-enabled genomic modification is initiated by the N-terminal nCas9 that introduces genomic nicking in a programmable manner based on the pegRNA spacer sequence. The directed nicking via the Cas9 nickase provides the first layer of genomic verification that results in the release of the 3′ flap domain at the genomic locus. The 3′ flap domain is in turn designed to be complementary to the 3′ PBS domain of the pegRNA. The hybridization of the genomic 3′ flap to the pegRNA PBS domain provides a second layer of genomic verification. This hybridization event initiates the targeted reverse transcription of the desired sequence through the RTT domain. The natively synthesized 3′ flap domain is further resolved into the genomic target locus by the endogenous DNA repair pathway².

Despite the functionality of prime editing in making targeted genomic modifications, the editing of the targeted strand results in the creation of a DNA heteroduplex product, which has at least one mismatch between the strands and in turn leads to recognition by the Mismatch Repair (MMR) pathway. The MMR pathway either resolves the error by retaining the edited or non-edited product. This event inherently reduces the efficiency of prime editing in MMR competent cells. To overcome this issue, the PE3/PE5 systems utilize a nicking sgRNA designed against the non-edited strand to favor the edited over non-edited genomic product, although this can result into DDBs and lead to indel formation that may raise additional safety concerns^{2,6}. Because of this effect, the use of PE2 is generally preferred over PE3/PE5 both due to ease of design and safety concerns⁹.

Parallel efforts in the field have led to creation of PE4/PE5 system, which involved the co-expression of a dominant-negative MMR protein (MLH1d) to impede with the endogenous MMR pathway⁶. Although the suppression of the MMR activity can favor for the genomic alteration, the long-term inhibition of MMR can lead to undesired mutations across the genome⁸. In addition, the delivery of the MLH1d in the PE4/PE5 system increases the size of the payload for therapeutic applications.

To overcome some of the safety concerns seen in the PE3, PE4 and PE5 systems, further improvement in PE engineering had modified the PE2 domains for development of the PEmax⁷ system. The engineered PEmax protein contains additional mutations in the nCas9 domain and bears a human codon-optimized RT domain. To enhance the nuclear delivery of the payload, PEmax contains a C-terminal c-Myc NLS and an additional 34-aa linker between the nCas9 and RT domain that encodes for an SV40 NLS. The use of PEmax can improve editing efficiency without bystander effects and has been applied toward genome modification applications in vitro and in vivos.

Parallel advances in improving PE functionality have led to the development of an engineered pegRNA (epegRNA) platform⁸. The epegRNA makes use of a 3′ nuclease-resistant pseudoknot structure that overcomes exonuclease degradation and in turn protects the 3′ PBS domain from truncation, which could be detrimental to PE functionality. The use of epegRNA requires an additional in silico design tool (pegLIT) that can be used for the screening of a linker domain and iterative screening of different pseudoknot structures (mpknot and TevopreQ1) that together can omit secondary structure crosstalk barrier within the pegRNA sequence.

Additional split PE paradigms have been developed through the efforts of Sontheimer et al. leading to the creation of a split PE system that utilizes a nicking sgRNA and separate prime-editing template (petRNA)¹⁰. The petRNA supplies the PBS and RTT domains using a MS2 bearing circularized PE template that directs Cas9 nickase using sgRNA nicking and enables MCP mediated M-MMLV RT to derive the genomic synthesis. To increase the efficiency for the split system, this platform is coupled with an additional sgRNA for nicking of the non-edited DNA strand and have been applied for AAV enabled in vivo delivery in mouse model.

Despite previous advances in PE engineering, the use of this platform has been found to have a low efficiency in MMR-competent cells that account for most the known therapeutic cell types. As a result, the use of PE across tissue types shows low efficiency in vivo³. Additionally, the editing of primary cells with PE has been found to be challenging² and requires chemical synthesis of the epegRNA that bears 2′O-methyl group and phosphorothioate base pairs on the first and last three nucleotides^{2,8}. Not to mention, the existing state-of-the-art chemical synthesis results into low-yield pegRNAs that are longer than 100 nucleotides due to presence of truncated byproducts which can perturb pegRNA functionality¹⁰. Together this restricts PE use for ex vivo and in vivo therapeutic applications and better pegRNA systems are required to further advance PE into clinical trials.

Herein, the inventors investigated whether the use of circularized pegRNA can improve PE functionality by protecting the pegRNA from exonuclease degradation and providing prolonged genomic modification tool that can be beneficial for use in MMR competent cells. The lack of 5′ and 3′ ends on the circularized pegRNA system was additionally sought to mitigate immunogenicity concern that can exist within the linear pegRNA systems.

### Results

Circularized pegRNAs Enhance Prime Editing

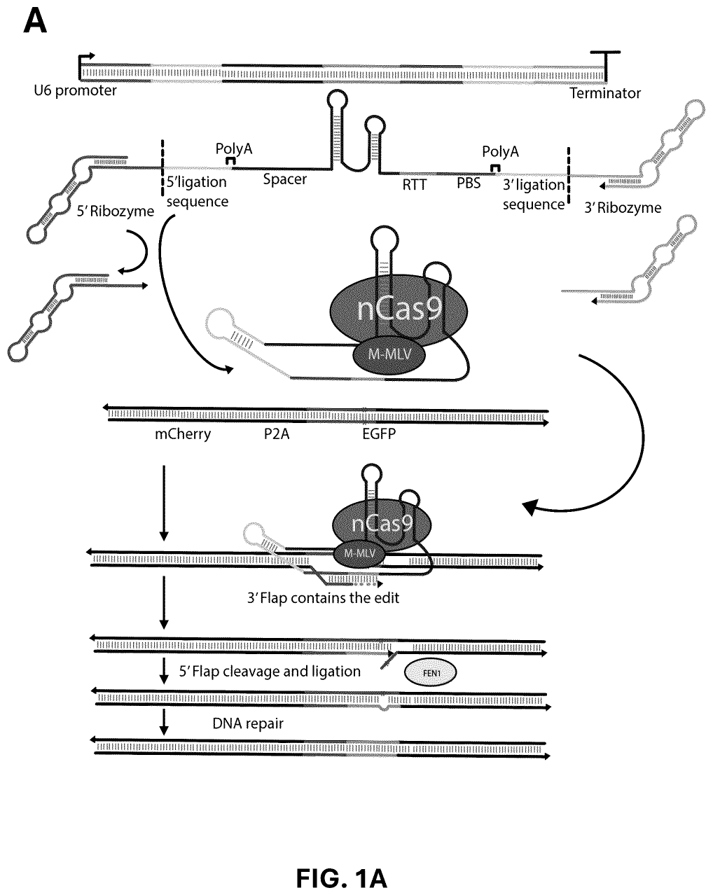

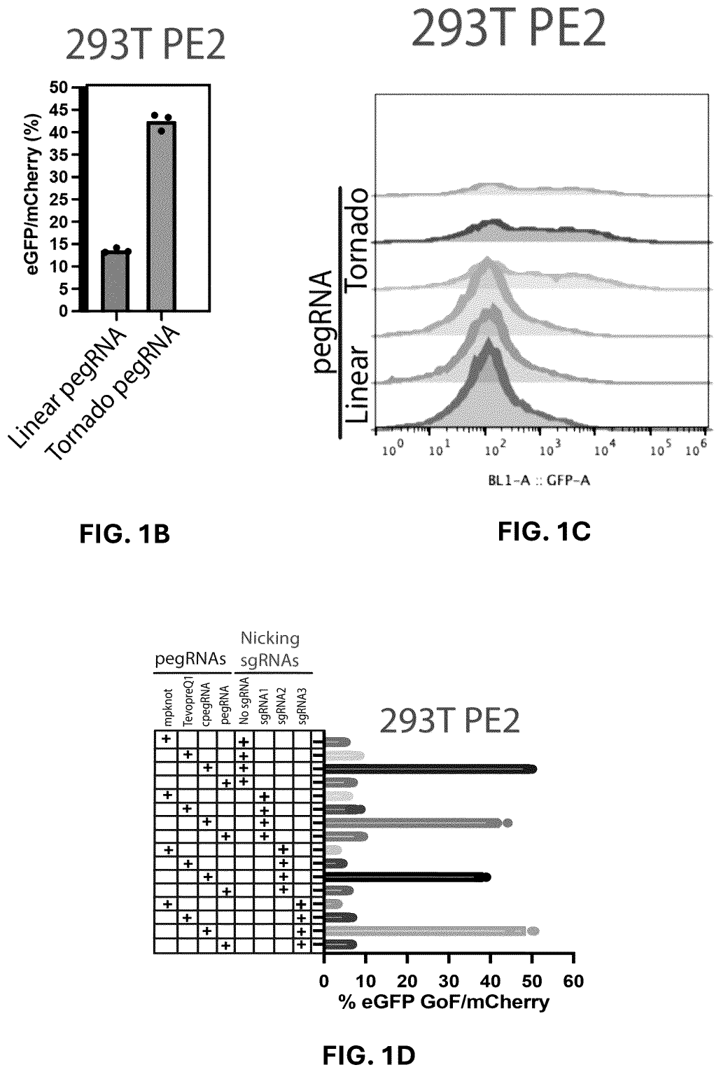

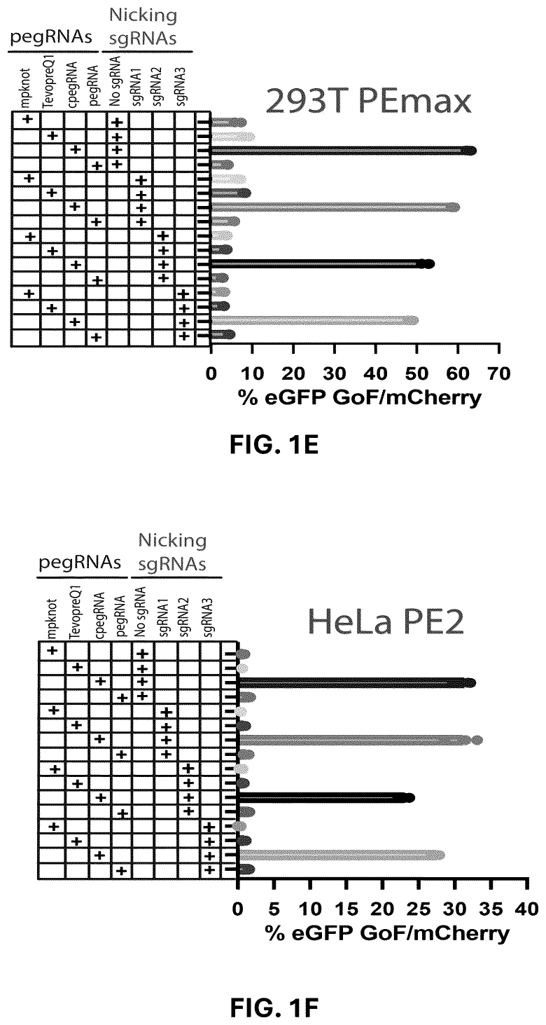

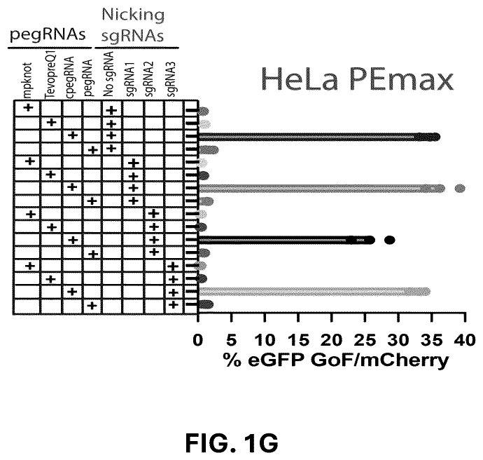

To increase pegRNA half-life, we attempted to develop a prime editing system that employed circularized pegRNAs (cpegRNAs). These circularized transcripts would present no ends to promote exonuclease degradation, thus increasing lifetime and potentially providing reduced immunogenicity¹¹. To generate the circularized RNA, we made use of the Tornado system, which drives self-ligation of the pegRNA using endogenously expressed RtcB ligase¹² (FIG. 1A) To promote proper cpegRNA function and mitigate steric effects, we flanked the 5′ spacer and the 3′ PBS domains with polyA sequences that are followed by ligation and ribozyme domains. Upon expression of the transcript, the 5′ and 3′ ribozyme domains undergo autocleavage to generate substrates (5′ hydroxyl and a 2′, 3′-cyclic phosphate) for ubiquitously expressed RtcB ligase that assists with the circularization event. The use of polyA sequence further provided a neutral secondary structure on the 5′ spacer and 3′ PBS domains to retain proper pegRNA functionality. This is because previous studies have shown that Cas9 does not tolerate 5′ extensions to the gRNA^{13, 14}.

The inventors initially compared cpegRNA to linear pegRNA by designing them against an exogenous reporter plasmid that encodes for mCherry and mutant EGFP containing an in-frame stop codon to be corrected by the PE. The mCherry and mutant EGFP were expressed from a single transcript using a P2A self-cleaving peptide. The linear and circularized pegRNAs both contain universal PBS (14 nt) and RTT (13 nt) domains and are designed against the EGFP stop-codon on the reporter plasmid. Using flow cytometry on HEK 293T cells, the inventors determined the efficiency of gene modification that results in base-substitution of the EGFP stop codon (TAG->TGG). The percentage of the EGFP-positive cells that were gated for the mCherry transfection control increased from 10% to 45% when the pegRNA was placed within the Tornado backbone (FIG. 1B). The base-substitution generated from the cpegRNA leads to a substantial shift in the EGFP histogram (FIG. 1C). Together these results indicate that the cpegRNA can be used for base-substitution on an exogenous plasmid. This result also provides evidence that the 5′ spacer, 3′ RTT and PBS domains can retain their functionality within the circularized backbone.

To compare the cpegRNA against the existing PE systems, the inventors combinatorially tested epegRNAs (mpknot and TevopreQ1) with nicking sgRNAs against the reporter plasmid. Using the PE2/PE3 molecular machinery in HEK 293T cells, the cpegRNA was found to increase EGFP gain-of-function (GoF) from ˜10% to ˜50% compared to the linear pegRNA counterparts (FIG. 1D). The use of PEmax in 293T cells further increased EGFP GoF from ˜10% to ˜60% (FIG. 1E). These results establish the significant performance improvement of the cpegRNA against the (e)pegRNA system in MMR-deficient 293T cells, providing a ˜5-6-fold increase in PE efficiency.

With the aim of comparing the cpegRNA efficiency against the epegRNA in MMR-competent cells, the inventors repeated the combinatorial experiment in HeLa cells. The use of PE2/PE3 with cpegRNA backbone increased EGFP GoF from ˜1% to ˜30% compared to its linear pegRNA counterpart (FIG. 1F). Further coupling of the PEmax with the cpegRNA backbone increased EGFP GoF from ˜1% to ˜35% (FIG. 1G). Thus, cpegRNA provides a remarkable increase in prime editing in MMR-competent human cells, yielding a ˜30-35-fold increase in the efficiency compared to the (e)pegRNA system.

### Split PE Strategy

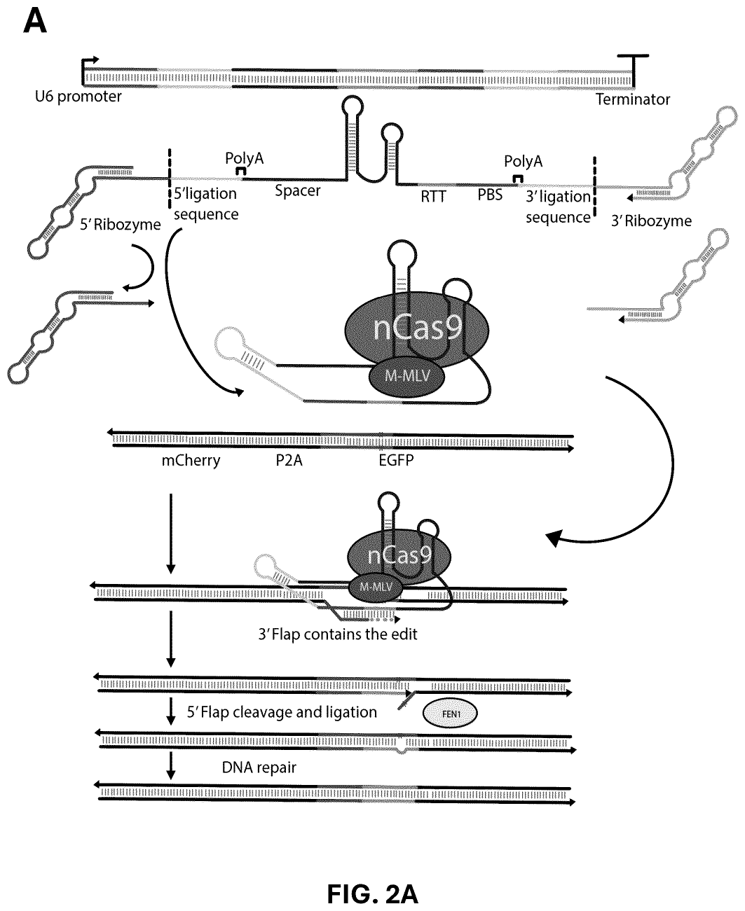

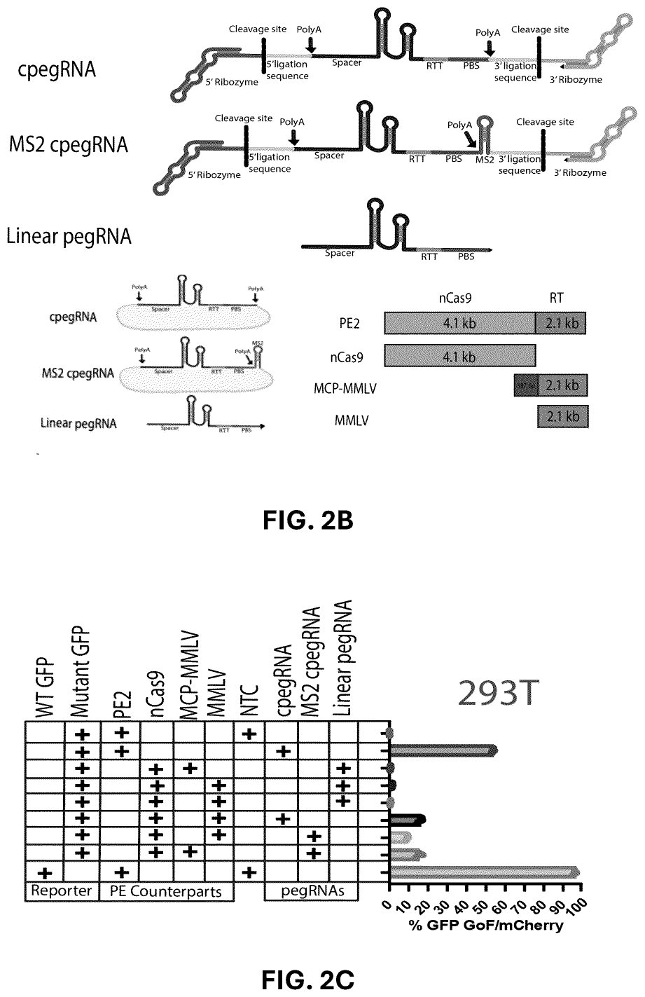

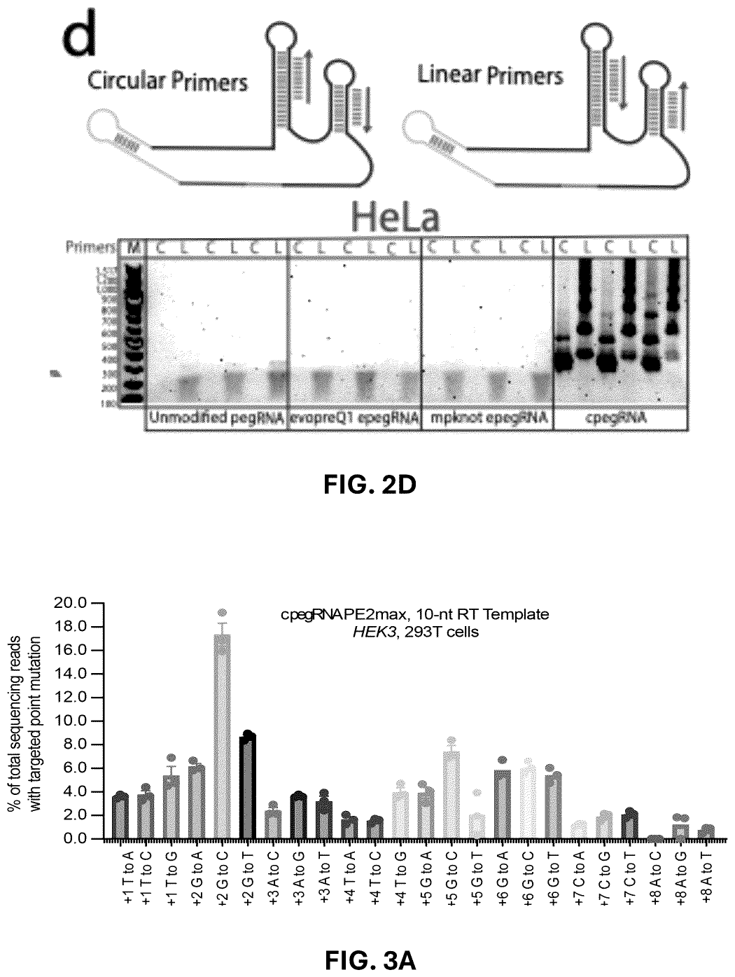

To enable the delivery of the PE systems compatible with the restricted AAV packaging size, the inventors investigated whether cpegRNAs could be coupled with split PE constructs to edit an exogenous reporter plasmid. Previously the use of the split PE system was coupled with a nicking sgRNA and an MS2 petRNA for recruitment of the MCP-RT domain and directed genomic synthesis in vivo¹⁰. To assess the functionality of the split PE system with the cpegRNA, the inventors coupled nCas9 either with MMLV RT or MCP-MMLV RT domains and then combinatorially compared the split PE constructs to cpegRNA; MS2-cpegRNA, a circularized pegRNA featuring an MS2 to recruit MCP-MMLV RT; and linear pegRNA (FIG. 2A). The cpegRNA was able to drive EGFP GoF in 293T cells at ˜17% efficiency when coupled with nCas9 and RT split domains, whereas the linear pegRNA generated negligible EGFP expression (FIG. 2B). Unexpectedly, the inventors found that the cpegRNA achieved similar performance to the MS2-cpegRNA, indicating that the MS2 domain is not necessary for efficient prime editing using the split PE and cpegRNA system. Taken together, these results indicate that the cpegRNA can more efficiency recruit the nCas9 and RT for AAV delivery applications, which overcomes the need for MCP and MS2 domains, reducing the size of the payload and overcoming possible immunogenicity concern.

Verifying cpegRNA Circularization

To verify for the circularization of the cpegRNA system, RT-PCR primers were designed either inward or outward on the sgRNA scaffold interrogate the effectiveness of the circularization. As expected, the linear primers provide the desired RT-PCR product on both the linear and cpegRNA counterparts, while the circular primers only provide concatemeric bands on the cpegRNA counterpart (FIG. 2C). Together, this verifies for the circularization of the pegRNA using the Tornado system. The increased RT-PCR band intensity seen in the cpegRNA as compared to the weak band intensity seen in the linear pegRNA, suggests that the concenration of the cpegRNA may be higher due to the prolong half-life.

### Genomic Modification Verification of the Circularized and Rotated-pegRNA

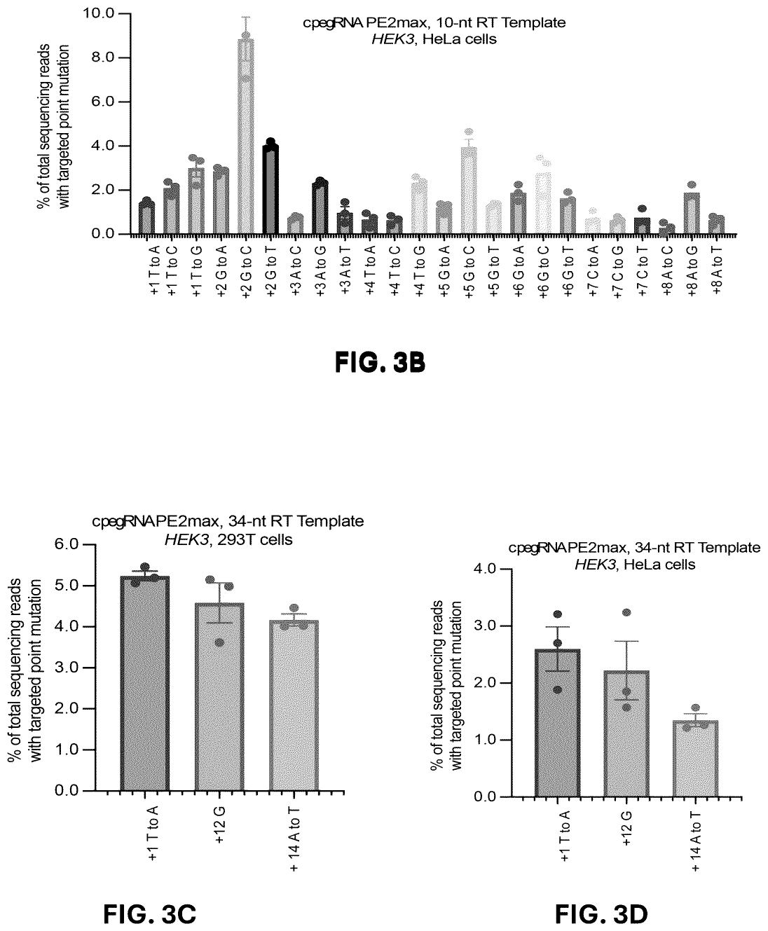

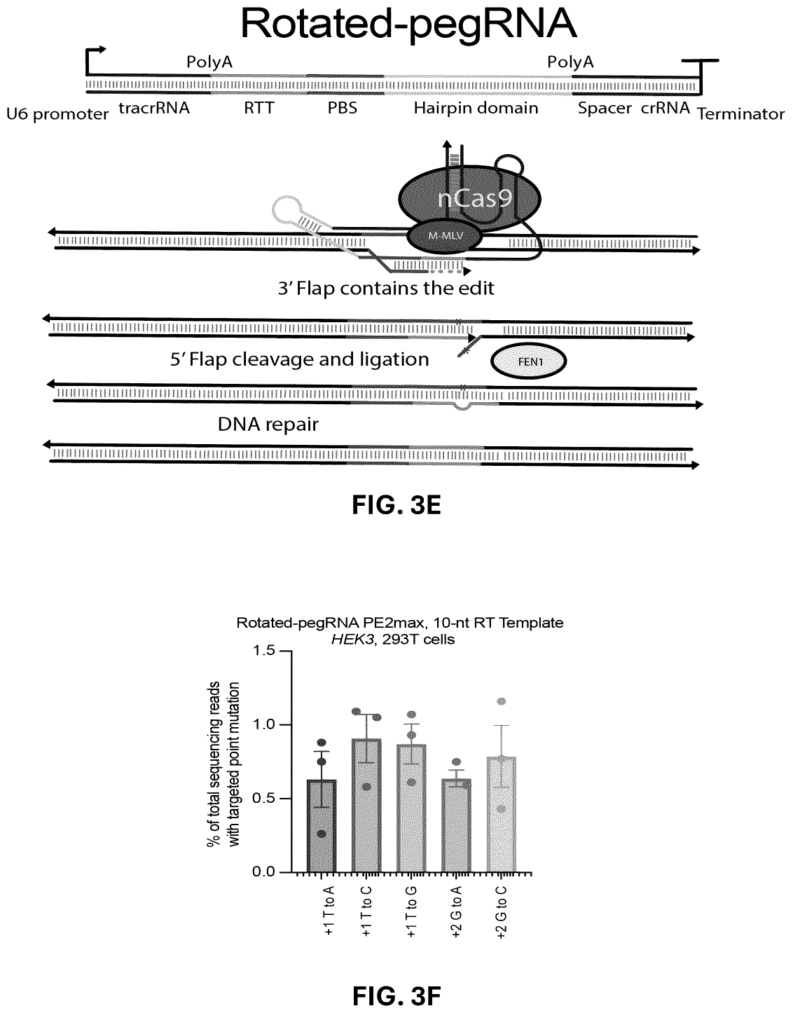

To verify the use of cpegRNAs for genomic modification, the inventors made use of functionally verified spacer, RTT and PBS pegRNA domains against HEK3 loci (2) and assessed the frequency of all the 12 possible types of base substitutions using the PE2max system. The use of cpegRNAs provided the desired genomic modification in HEK 293T and HeLa cells using the 10-nt RT template (FIG. 3A-3B). To examine longer-range genomic modifications, we made use of a 34-nt RT template embedded in the cpegRNA and assessed genomic alterations in HEK 293T and HeLa cells (FIG. 3C-3D). The genomic modification using cpegRNA does not provide any bystander editing at the genomic modification loci (FIG. X-X), ** Alex and Milad please let us know if you will be adding FIG. S3 mentioned in the manuscript draft** but varies in efficiency based on the RT template. Together we confirmed the functionality of cpegRNAs in MMR-deficient HEK 293T cells and MMR-competent HeLa cells.

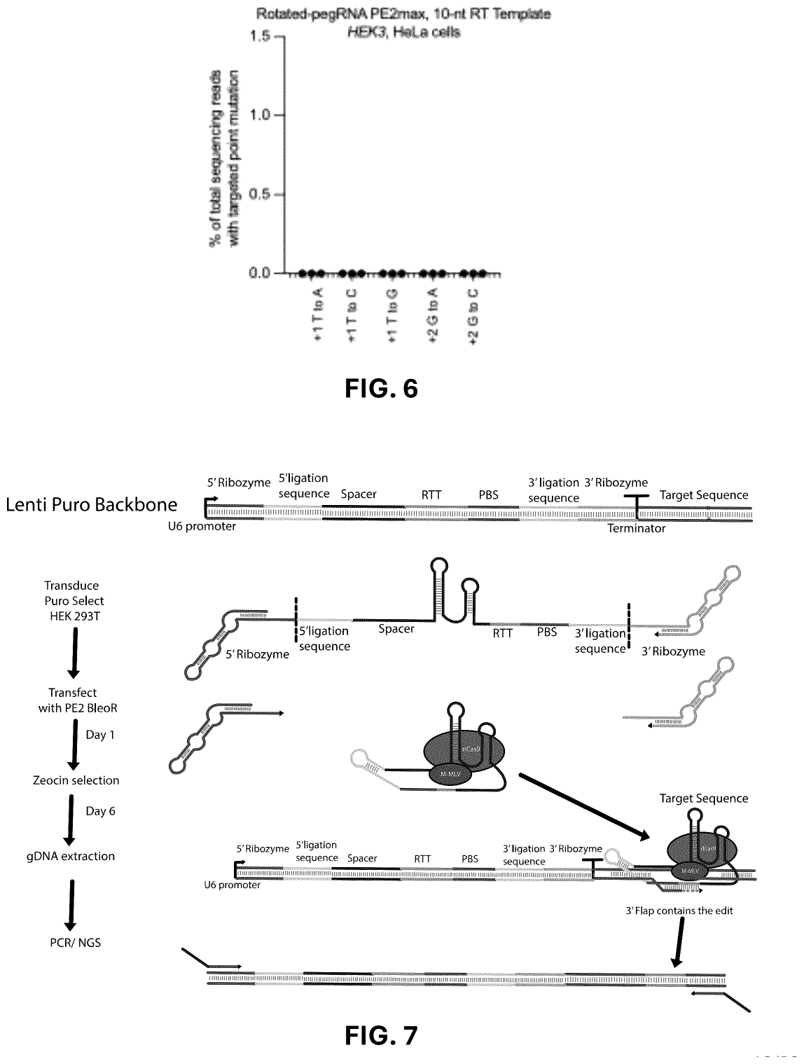

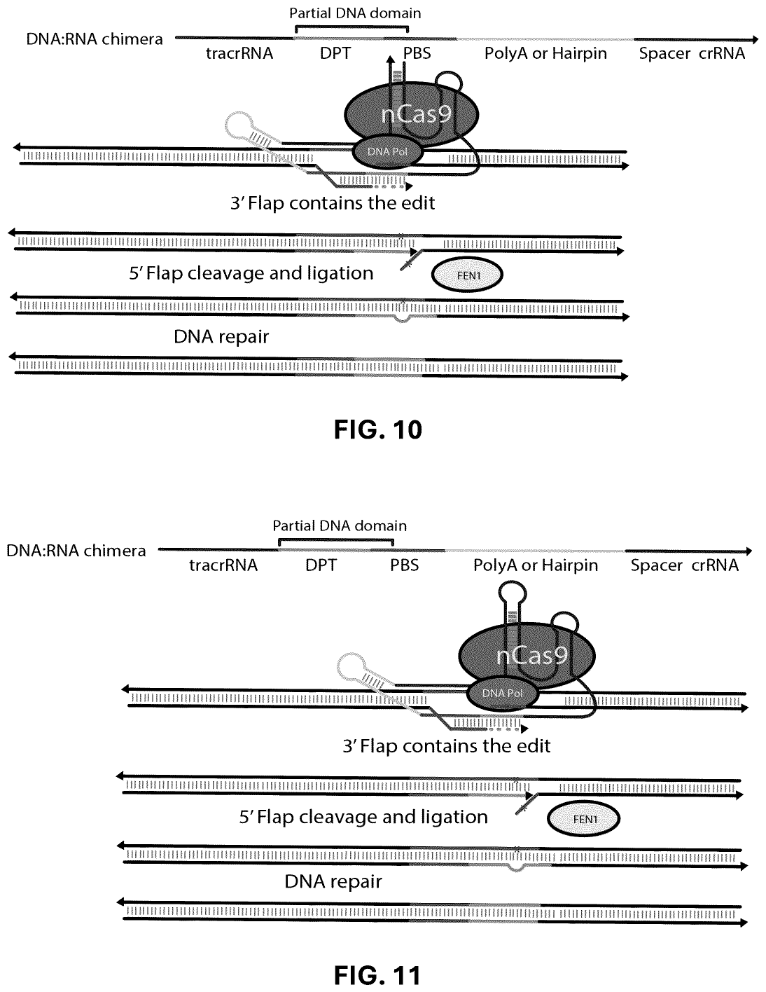

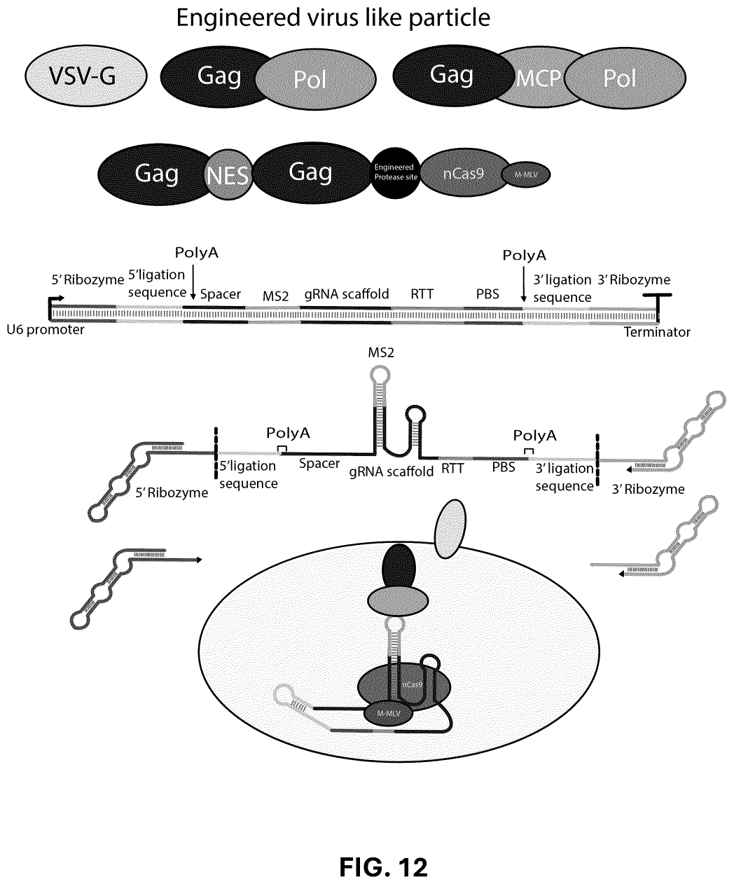

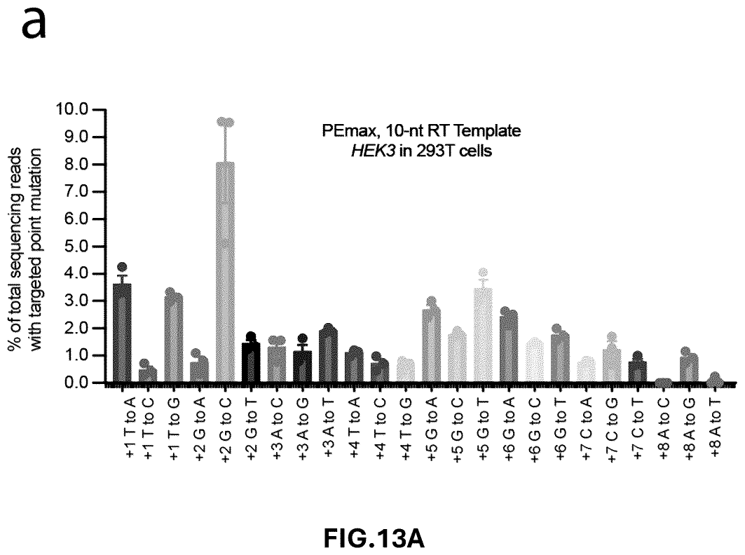

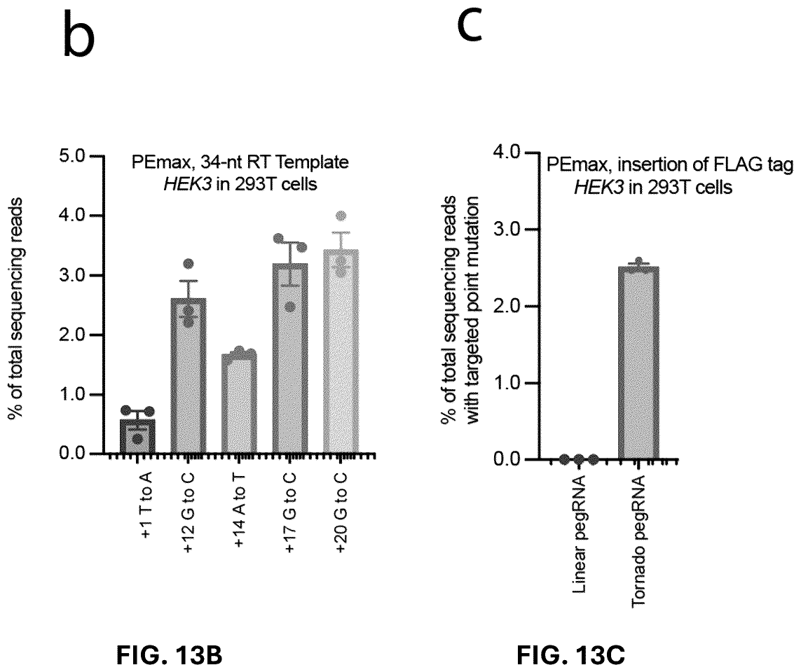

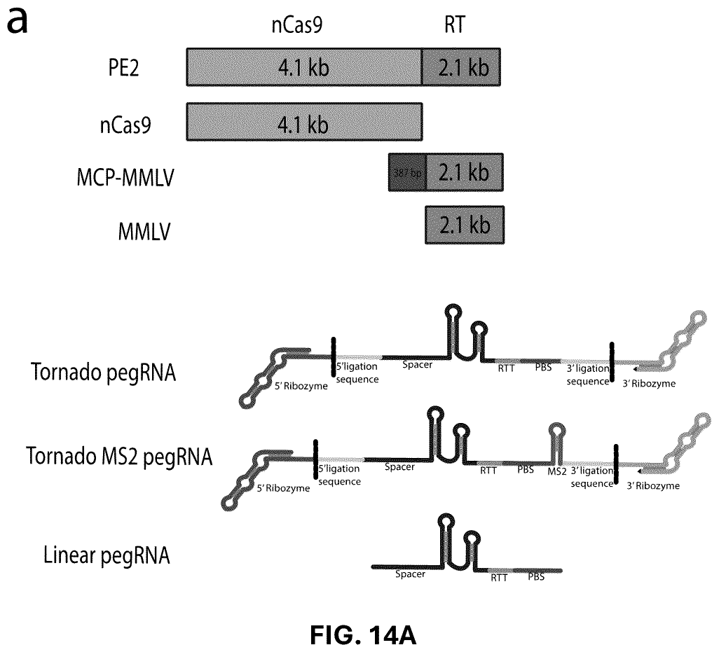

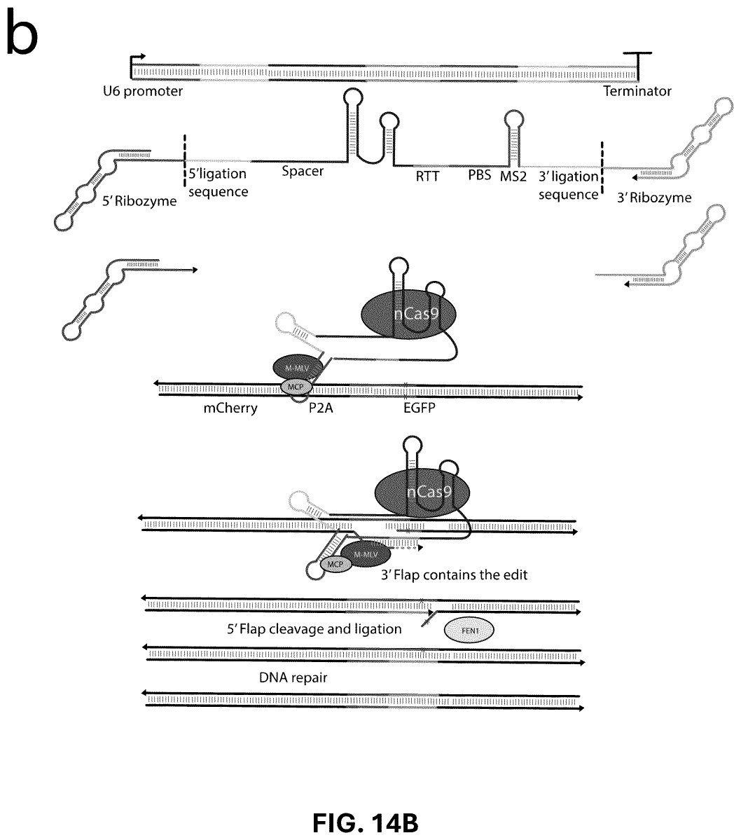

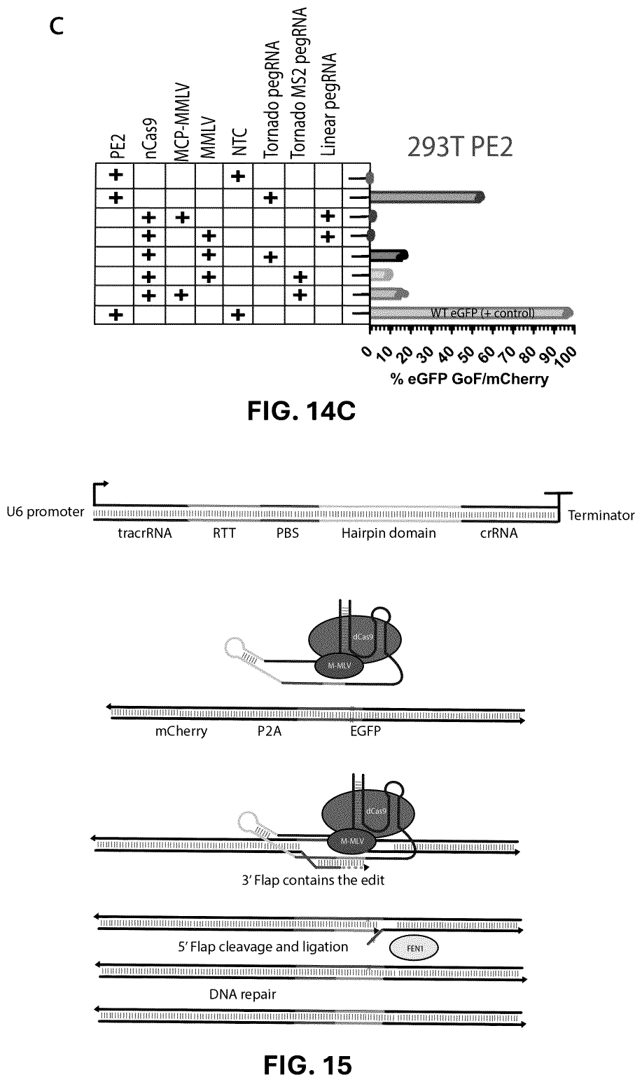

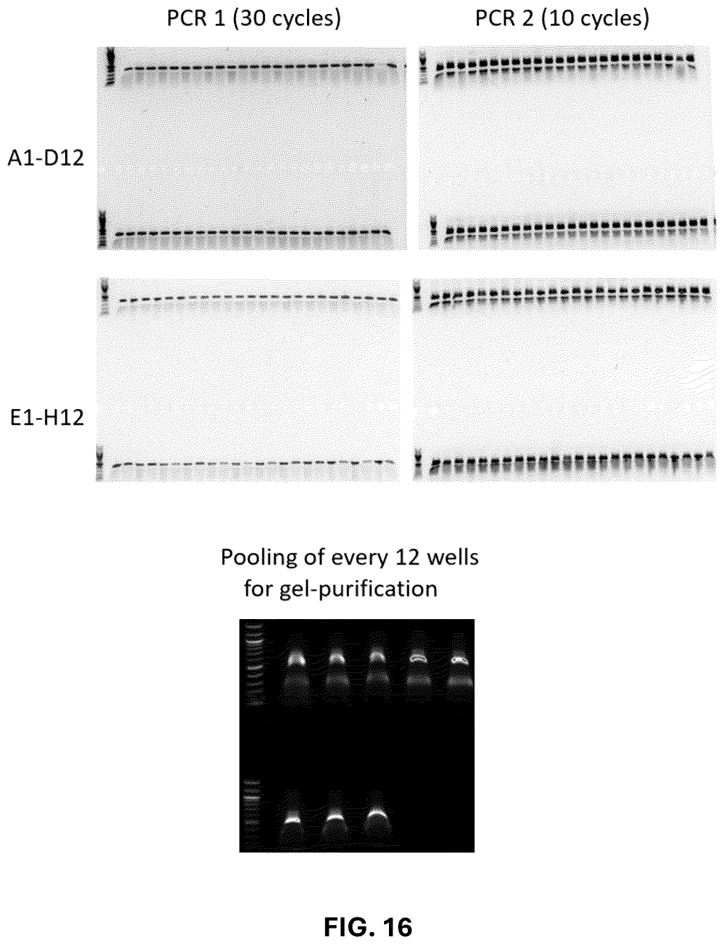

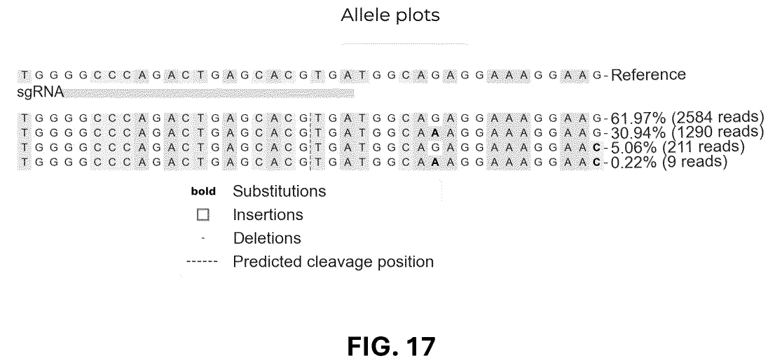

To determine if the improved performance of cpegRNAs was due to changes in backbone sequence rather than circularization, the inventors also studied a nicked version of the cpegRNA that retained the cpegRNA post-ligation backbone but featured a nick at the top of the stem formed between the crRNA and tracrRNA in wild-type cas9 guides (FIG. 3E). This linear circularly permutated version of the pegRNA is also referred to as a rotated pegRNA (rpegRNA). The rpegRNA was able to establish the desired alterations in HEK3 loci of 293T cells with a modest efficiency (FIG. 10F) compared to the cpegRNA counterpart (FIG. 3A-3D). However, the rpegRNA was not able to induce for the same edits in the HeLa cell line (FIG. 6) which provides further evidence of the benefits cpegRNA provides for genomic modification in MMR competent cells and demonstrates the importance of circularization for this effect.

Comparing of the cpegRNA to Engineered pegRNA

To compare the functionality of the circularized pegRNA to the state-of-the-art engineered pegRNA systems, we used PEmax to target three genomic loci (DMNT1, RUNX and VEGFA) using linear unmodified pegRNA, evopreQ1 epegRNA, mpknot epegRNA and circularized cpegRNA in HEK 293T cells (FIG. 4B-4D) and HeLa cells (FIG. 4E-4G). Although the use of PE2max and cpegRNA provided the desired edits in both cell-types, the linear (e)pegRNA systems could not provide any of the intended genomic modifications using identical transfection conditions as the cpegRNA. These results provide further evidence that cpegRNAs can be coupled with PE2 for genome editing in MMR-competent cells and do not require the nicking sgRNA leveraged in PE3 and PE5 systems that can promote indel formation.

### DISCUSSION

In this study, we demonstrated that circularized pegRNAs provide substantial improvements in prime-editing functionality using the PE2 system and demonstrated superior editing activity compared to their linear epegRNA counterparts in MMR-competent and MMR-deficient cell lines. Notably, the improved performance in MMR-deficient cells for cpegRNAs was achieved without the additional nicking sgRNA required for PE3/PE5 systems, eliminating a key source of indels. The cpegRNAs can also minimize possible immunogenicity concerns that may persist in linear (e)pegRNA systems due to bearing naked 5′ and 3′ ends¹¹. The increased editing rates of cpegRNAs also extends to split PEs consisting of nCas9 and MMLV-RT, suggesting that the increased lifetimes, concentrations, and reduced immunogenicity of circularized can enhance PE efficiency in general.

We expect that cpegRNA can be explored across a diverse set of PE applications that currently suffer from low genomic editing efficiency. For instance, the ex vivo engineering of patient primary cells are currently found to be difficult using the existing state-of-the-art chemically synthesized epegRNA system⁸. As such, alternative strategies to pursue PE in the ex vivo setting can be beneficial for autologous applications.

Other ex vivo applications that could benefit from cpegRNAs system may include integration of gene-sized cargo. There had been several PE enabled approaches that can embed gene-sized cargo within precise loci in the genome, namely TwinPE¹⁵, PASTE¹⁶, and TJ-PE¹⁷, and other dual pegRNA approaches that include PrimeDel¹⁸, PEDAR¹⁹, dual-pegRNAs²⁰, HOPE²¹, GRAND²² and Bi-PE²³. The existing gene-sized integration system may all benefit from the use of circularized pegRNA strategy that may harness their improved editing efficiencies.

For in vivo applications, the utility of the circularized pegRNA can be examined for AAV packaging that benefits from the use of split PE counterparts due to the limited size of the payload³. Alternatively, the use of circularized pegRNA system can be harnessed for LNP delivery of the cargo in the lung and liver. In this context, the circular pegRNA system can provide for long-lasting pegRNA functionality and obviate immune recognition due to the absence of naked 5′ and 3′ ends. Additional use of the circularized pegRNA can be explored with the engineered virus-like particles (eVLPs) that can bear cell-type specific recognition^{24,25}.

### Materials and Methods

Plasmid preparation: The reporter plasmid (pCMV_mCherry_P2A_EGFP*) was synthesized using Twist Bioscience clonal synthesis service. The pegRNAs, epegRNAs and sgRNAs were cloned using the Integrated DNATechnologies (IDT) MiniGene clonal synthesis service and further grew up using GenScript High Throughput (HT) Plasmid Prep service at 10 μg scale. The PE2 (Addgene #132775), PEmax (Addgene #174820), nCas9 (Addgene #51129) MMLV-RT (Addgene #181801), MCP-M-MLV-RT (Addgene #181799) were inoculated in 45 mL of Terrific Broth (Sigma Aldrich T0918) and grown overnight at 37° C. in the presence of antibiotic. The plasmids were extracted using Midiprep kit (Zymo Research Cat #D4213) according to the manufacturer's instructions.

Cellular maintenance: The 293T (ATCC CRL-3216) and HeLa (ATCC CCL-2) cell lines were kept at 37° C. and 5% CO₂ incubator and grown in Dulbecco's modified Eagle's medium (DMEM) (Thermo Fisher Scientific) that was supplemented with 10% heat-inactivated fetal-bovine serum (Thermo Fisher Scientific), 50 Ul/ml penicillin, and 50 μg/mL streptomycin.

Transfection for Flow Cytometry: The stock of PEI for transfection was made from polyethyleneimine (Polysciences 23966-2) and dissolved in deionized water at 0.323 g/L with the help of hydrochloric acid and sodium hydroxide. Upon dissolving PEI, the stock was filtered (0.22 μm) for sterilization. The PEI stocks were stored at −80′ C for use after thawing at room temperature. For 96-well transfection, 8 μL of plasmid DNA (50 ng/ul) was brought up to 20 μL using 0.15 M sodium chloride (NaCl). The DNA/NaCl was then pre-mixed with 20 μL of PEI-NaCl that was supplied from a master mix (80 μL PEI+420 μL 0.15 M NaCl). The 40 μL PEL: DNA mixture was incubated at room temperature for 10 minutes and 10 μL were added to per each well that were seeded at 5,000-15,000 cells the day before.

Transfection for Genomic Modification and RT-PCR: The 293T and HeLa cells were seeded one day prior to the transfection with 15,000-20,000 cells per each 96-well. Cells were transfected with jetOPTIMUS (Polyplus Transfection CAT #101000025) using the manufacture's protocol. For 96-well transfection, a total of 100 ng was used per well that supplied prime-editor and pegRNA in 1:1 ratio. Briefly 500 ng of DNA mix was diluted in 50 μL of jetOPTIMUS buffer followed by addition of 0.5 μL jetOPTIMUS, 10-minutes incubation at room-temperature and addition of the 10 μL transfection mix per well.

pegRNA design:The pegRNA design was undertaken using PrimeDesign tool and PegLIT software to design a linker domain and suitable 3′ pseudoknot (mpknot and TevopreQ1) structure.

RNA purification and RT-PCR: The RNA purification was performed after 48 hours of transfection using Bioresearch Technologies QuickExtract RNA Extraction Kit (CAT #QER090150) in 96-well format according to manufacturer's protocol with the following changes. Briefly, the adherent cells were carefully washed with 50 μL of pre-warmed PBS prior to addition of 50 μL ice cold QuickExtract RNA Extraction solution used for resuspension of the cells by pipetting. The RNA lysate was preheated at 65° C. for 2-minutes prior to addition DNase I treatment according to manufacturer's protocol. Following RNA purification, the NEB ProtoScript II First Strand cDNA Synthesis (CAT #E6560H) was used with random primers to derive cDNA synthesis. The resulting cDNA was used in a PCR reaction using NEB Ultra II Q5 Master Mix (CAT #M044L) where the reaction was incubated as follows: 98° C. for 3 minutes followed by 32 cycles of (98° C. for 10 s, 65° C. for 20 s and 72° C. for 30 s) and finishing with a final extension at 72° C. for 10 minutes.

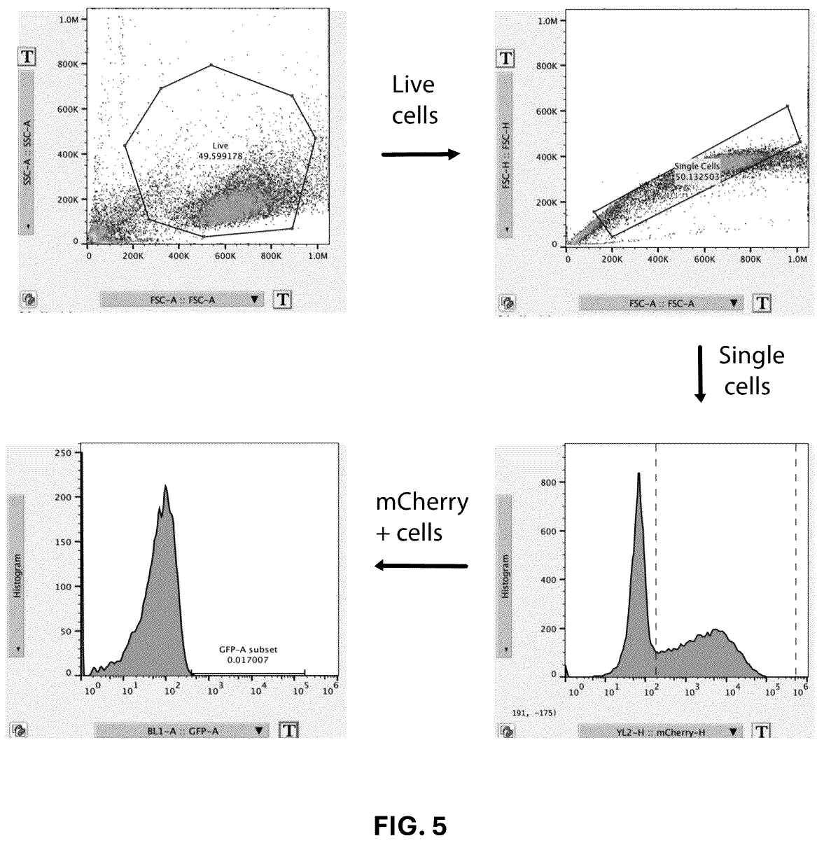

Flow cytometry: Flow cytometry was performed using 293T or HeLa cell lines that were prepared by trypsinization (0.05% Trypsin-EDTA Thermo Fisher) and resuspension in full media (DMEM+10% FBS). The Life Technologies Attune Nxt 4-laser acoustic focusing flow cytometer was used for flow cytometry. The FlowJo software tool was then used for gating of live cells, single cells, mCherry and eGFP positive cells, where the mCherry was used as a transfection control while no pegRNA control was used to access for eGFP positive cells (FIG. 5).

Genomic Purification and Library Preparation: The genomic purification was performed in a 96-well plate format according to the established protocol (2) with the described modification. The QuickExtract™ DNA Extraction Solution (CAT #QE09050) was used for genomic purification according to manufacturer's protocol, briefly the cells were washed with 1×PBS in 96-well prior to addition of 50 μL of the genomic extraction solution and resuspension into PCR plate for incubation at 65° C. for 6-minutes and 98° C. for 2-minutes.

The PCR was performed with NEB Ultra II Q5 Master Mix (CAT #M044L) using 1.5 μL of the genomic extract per 25 μL reaction. The genomic PCR reaction was incubated as follows: 98° C. for 3 minutes followed by 30 cycles of (98° C. for 10 s, 67° C. for 20 s and 72° C. for 30 s) and finishing with a final extension at 72° C. for 10 minutes. The PCR2 barcoding was followed by adding 1 μL of the PCR1 product per 25 μL reaction in 96-well format, where the reaction was incubated as follows: 98° C. for 3 minutes followed by 10 cycles of (98° C. for 10 s, 67° C. for 20 s and 72° C. for 30 s) and finishing with a final extension at 72° C. for 10 minutes. After barcoding, every 24 PCR2 were pooled via addition of 2 μL of the barcoded product followed by the gel purification using Wizard SV Gel and PCR Clean-Up System (CAT #A9282). The concentration of the gel-purified products was determined using Thermofisher Qubit 4 (CAT #33238) and Quibit 1×dsDNA BRAssay prior to normalizing each library to 8 nM final concentration. The pooled products underwent NGS using MiSeq system (MS-410-1003) with MiSeq Reagent Kit v2 (300-cycles) (MS-102-2002). The single-end reads were demultiplexed and CRISPResso2 tool was used to analyze the data²⁴.

TABLE 1
Sequences of pegRNAs and sgRNAs used in mammalian cell experiments. (All
sequences shown are in 5′ to 3′ orientation, wherein the pegRNA is composed of the spacer
sequence, the sgRNA scaffold, and the 3′ extension (contains PBS and RTT template) and
in the case of pegRNA there is a 3′ motif.)

| 4-Apr-25 | | |
|---|---|---|
| 12/31/24 | | |
| 94 | | |
| 13.4285714 | | |
| 14.7714286 | | |
| SEQ ID | | |
| NO: | | Seqeunce |
| 1 | sgRNA | gttttagagctagaaatagcaagttaaaataaggctagtccgttatcaacttgaaaaag |
| | scaffold | tggcaccgagtcggtgc |
| | sequence | |
| | (5′ to 3′) | |
| 2 | 5′ Twister | gagggcctatttcccatgattccttcatatttgcatatacgatagcttaccgtaacttgaaa |
| | Ribozyme | gtatttcgatttcttggctttatatatcttgtggaaaggacgaaacaccgaaacaccgcc |
| | and 5′ | atcagtcgccggtcccaagcccggataaaatgggagggggcgggaaaccgccta |
| | Ligation | accatgccgactgatggcagaaaaaaaaaa |
| | Domain | |
| 3 | 3′ Twister | AAAAAAAAAGCTGCCATCAGTCGGCGTGGACTGTAGAACA |
| | Ribozyme | CTGCCAATGCCGGTCCCAAGCCCGGATAAAAGTGGAGGG |
| | and 5′ | TACAGTCCACGC |
| | Ligation | |
| | Domain | |
| 4 | cpegRNA | gagggcctatttcccatgattccttcatatttgcatatacgatagcttaccgtaacttgaaa |
| | Architecture | gtatttcgatttcttggctttatatatcttgtggaaaggacgaaacaccgaaacaccgcc |
| | | atcagtcgccggtcccaagcccggataaaatgggagggggcgggaaaccgccta |
| | | accatgccgactgatggcagaaaaaaaaaagttttagagctagaaatagcaagtta |
| | | aaataaggctagtccgttatcaacttgaaaaagtggcaccgagtcggtgcaaaaaa |
| | | aaagctgccatcagtcggcgtggactgtagaacactgccaatgccggtcccaagcc |
| | | cggataaaagtggagggtacagtccacgc |
| 5 | Rotated | CGGCAGCATAGCAAGTTAAAATAAGGCTAGTCCGTTATCA |
| | pegRNA | ACTTGAAAAAGTGGCACCGAGTCGGTGC |
| | Architecture | AAAAAAAAAGCTGCCATCAGTCGGCGTGGACTGTAGAACC |
| | | ATGCCGACTGATGGCAGAAAAAAAAAA |
| | | GTTTTAGAGCTATGCTGCCG |

TABLE 2

| pegRNA and | | | |
|---|---|---|---|
| nicking | | | epegRNA with |
| sgRNA | Spacer sequence | 3′ extension | linker |
| FIG. 1b and c | GTCGTCACTAGT | GCCTGTTCCGTGGCCGAC | |
| pegRNA_GFP- | GTCGGCTA (SEQ | ACTAGTGAC (SEQ ID NO: | |
| GOF_RTT13_ | ID NO: 6) | 7) | |
| PBS14 | | | |
| FIG. 1b and | GTCGTCACTAGT | GCCTGTTCCGTGGCCGAC | AAGGAATTGGGTC |
| cpegRNA_GFP- | GTCGGCTA (SEQ | ACTAGTGAC (SEQ ID NO: | AGGAGCCCCCCC |
| GOF_RTT13_ | ID NO: 6) | 7) | CCTGAACCCAGG |
| PBS14_mpknot | | | ATAACCCTCAAAG |
| | | | TCGGGGGGCAAC |
| | | | CC (SEQ ID NO: 8) |
| FIG. 1e, f, g | GTCGTCACTAGT | GCCTGTTCCGTGGCCGAC | TAAAGGAACGCG |
| and h | GTCGGCTA (SEQ | ACTAGTGAC (SEQ ID NO: | GTTCTATCTAGTT |
| pegRNA_GFP- | ID NO: 6) | 7) | ACGCGTTAAACCA |
| GOF_RTT13_ | | | ACTAGAA (SEQ ID |
| PBS14_ | | | NO: 9) |
| TevopreQ1 | | | |
| FIG. 1e, f ,g | CTGGCAAACTGCCTGTTCCG | | |
| and h | (SEQ ID NO: 10) | | |
| nicking | | | |
| sgRNA1 | | | |
| FIG. 1e, f ,g | CTAGTGACGACGCTCTGCTA | | |
| and h | (SEQ ID NO: 11) | | |
| nicking | | | |
| sgRNA2 | | | |
| FIG. 1e, f ,g | CCAGTGCTTTTCAAGATACC | | |
| and h | (SEQ ID NO: 12) | | |
| nicking | | | |
| sgRNA3 | | | |
| FIG. 3a, c | GGCCCAGACTG | TCTGCCATCTCGTGCTCA | |
| HEK3_1TtoA | AGCACGTGA | GTCTG (SEQ ID NO: 14) | |
| | SEQ. ID NO: 13) | | |
| FIG. 3a, c | GGCCCAGACTG | TCTGCCATCGCGTGCTCA | |
| HEK3_1TtoC | AGCACGTGA | GTCTG (SEQ ID NO: 16) | |
| | (SEQ ID NO: 15) | | |
| FIG. 3a, c | GGCCCAGACTG | TCTGCCATCCCGTGCTCA | |
| HEK3_1TtoG | AGCACGTGA | GTCTG (SEQ ID NO. 17) | |
| | (SEQ ID NO: 15) | | |
| FIG. 3a, c | GGCCCAGACTG | TCTGCCATTACGTGCTCA | |
| HEK3_2GtoA | AGCACGTGA | GTCTG (SEQ ID NO. 18) | |
| | (SEQ ID NO: 15) | | |
| FIG. 3a, c | GGCCCAGACTG | TCTGCCATGACGTGCTCA | |
| HEK3_2GtoC | AGCACGTGA | GTCTG (SEQ ID NO. 19) | |
| | (SEQ ID NO: 15) | | |
| FIG. 3a, c | GGCCCAGACTG | TCTGCCATAACGTGCTCA | |
| HEK3_2GtoT | AGCACGTGA | GTCTG (SEQ ID NO. 20) | |
| | (SEQ ID NO: 15) | | |
| FIG. 3a, c | GGCCCAGACTG | TCTGCCAGCACGTGCTCA | |
| HEK3_3AtoC | AGCACGTGA | GTCTG (SEQ ID NO: 21) | |
| | (SEQ ID NO: 15) | | |
| FIG. 3a, c | GGCCCAGACTG | TCTGCCACCACGTGCTCA | |
| HEK3_3AtoG | AGCACGTGA | GTCTG (SEQ ID NO: 22) | |
| | (SEQ ID NO: 15) | | |
| FIG. 3a, c | GGCCCAGACTG | TCTGCCAACACGTGCTCA | |
| HEK3_3AtoT | AGCACGTGA | GTCTG (SEQ ID NO: 23) | |
| | (SEQ ID NO: 15) | | |
| FIG. 3a, c | GGCCCAGACTG | TCTGCCTTCACGTGCTCA | |
| HEK3_4TtoA | AGCACGTGA | GTCTG (SEQ ID NO: 24) | |
| | (SEQ ID NO: 15) | | |
| FIG. 3a, c | GGCCCAGACTG | TCTGCCGTCACGTGCTCA | |
| HEK3_4TtoC | AGCACGTGA | GTCTG (SEQ ID NO: 25) | |
| | (SEQ ID NO: 15) | | |
| FIG. 3a, c | GGCCCAGACTG | TCTGCCCTCACGTGCTCA | |
| HEK3_4TtoG | AGCACGTGA | GTCTG (SEQ ID NO: 26) | |
| | (SEQ ID NO: 15) | | |
| FIG. 3a, c | GGCCCAGACTG | TCTGCTATCACGTGCTCA | |
| HEK3_5GtoA | AGCACGTGA | GTCTG (SEQ ID NO: 27) | |
| | (SEQ ID NO: 15) | | |
| FIG. 3a, c | GGCCCAGACTG | TCTGCGATCACGTGCTCA | |
| HEK3_5GtoC | AGCACGTGA | GTCTG (SEQ ID NO: 28) | |
| | (SEQ ID NO: 15) | | |
| FIG. 3a, c | GGCCCAGACTG | TCTGCAATCACGTGCTCA | |
| HEK3_5GtoT | AGCACGTGA | GTCTG (SEQ ID NO: 29) | |
| | (SEQ ID NO: 15) | | |
| FIG. 3a, c | GGCCCAGACTG | TCTGTCATCACGTGCTCA | |
| HEK3_6GtoA | AGCACGTGA | GTCTG (SEQ ID NO: 30) | |
| | (SEQ ID NO: 15) | | |
| FIG. 3a, c | GGCCCAGACTG | TCTGGCATCACGTGCTCA | |
| HEK3_6GtoC | AGCACGTGA | GTCTG (SEQ ID NO: 31) | |
| | (SEQ ID NO: 15) | | |
| FIG. 3a, c | GGCCCAGACTG | TCTGACATCACGTGCTCA | |
| HEK3_6GtoT | AGCACGTGA | GTCTG (SEQ ID NO: 32) | |
| | (SEQ ID NO: 15) | | |
| FIG. 3a, c | GGCCCAGACTG | TCTTCCATCACGTGCTCAG | |
| HEK3_7CtoA | AGCACGTGA | TCTG (SEQ ID. NO: 33) | |
| | (SEQ ID NO: 15) | | |
| FIG. 3a, c | GGCCCAGACTG | TCTCCCATCACGTGCTCA | |
| HEK3_7CtoG | AGCACGTGA | GTCTG (SEQ ID NO: 34) | |
| | (SEQ ID NO: 15) | | |
| FIG. 3a, c | GGCCCAGACTG | TCTACCATCACGTGCTCA | |
| HEK3_7CtoT | AGCACGTGA | GTCTG (SEQ ID NO: 35) | |
| | (SEQ ID NO: 15) | | |
| FIG. 3a, c | GGCCCAGACTG | TCGGCCATCACGTGCTCA | |
| HEK3_8AtoC | AGCACGTGA | GTCTG (SEQ ID NO: 36) | |
| | (SEQ ID NO: 15) | | |
| FIG. 3a, c | GGCCCAGACTG | TCCGCCATCACGTGCTCA | |
| HEK3_8AtoG | AGCACGTGA | GTCTG (SEQ ID NO: 37) | |
| | (SEQ ID NO: 15) | | |
| FIG. 3a, c | GGCCCAGACTG | TCAGCCATCACGTGCTCA | |
| HEK3_8AtoT | AGCACGTGA | GTCTG (SEQ ID NO: 38) | |
| | (SEQ ID NO: 15) | | |
| FIG. 3b, d | GGCCCAGACTG | TGGAGGAAGCAGGGCTTC | |
| HEK3_1TtoA | AGCACGTGA | CTTTCCTCTGCCATCTCGT | |
| | (SEQ ID NO: 15) | GCTCAGTCTG (SEQ ID NO: | |
| | | 39) | |
| FIG. 3b, d | GGCCCAGACTG | TGGAGGAAGCAGGGCTTC | |
| HEK3_12GtoC | AGCACGTGA | CTTTGCTCTGCCATCACGT | |
| | (SEQ ID NO: 15) | GCTCAGTCTG (SEQ ID NO: | |
| | | 40) | |
| FIG. 3b, d | GGCCCAGACTG | TGGAGGAAGCAGGGCTTC | |
| HEK3 14AtoT | AGCACGTGA | CTATCCTCTGCCATCACGT | |
| | (SEQ ID NO: 15) | GCTCAGTCTG (SEQ ID NO: | |
| | | 41) | |
| FIG. 3f | GGCCCAGACTG | TCTGCCATCTCGTGCTCA | |
| HEK3_1TtoA | AGCACGTGA | GTCTG (SEQ ID NO: 14) | |
| | (SEQ ID NO: 15) | | |
| FIG. 3f | GGCCCAGACTG | TCTGCCATCGCGTGCTCA | |
| HEK3_1TtoC | AGCACGTGA | GTCTG (SEQ ID NO: 16) | |
| | (SEQ ID NO: 15) | | |
| FIG. 3f | GGCCCAGACTG | TCTGCCATCCCGTGCTCA | |
| HEK3_1TtoG | AGCACGTGA | GTCTG (SEQ ID NO. 17) | |
| | (SEQ ID NO: 15) | | |
| FIG. 3f | GGCCCAGACTG | TCTGCCATTACGTGCTCA | |
| HEK3_2GtoA | AGCACGTGA | GTCTG (SEQ ID NO. 18) | |
| | (SEQ ID NO: 15) | | |
| FIG. 3f | GGCCCAGACTG | TCTGCCATGACGTGCTCA | |
| HEK3_2GtoC | AGCACGTGA | GTCTG (SEQ ID NO. 19) | |
| | (SEQ ID NO: 15) | | |
| FIG. 4b, e | GATTCCTGGTGC | TCTGCCCTCCCGTCACCC | |
| DMNT1_FLAG- | CAGAAACA (SEQ | CTGTCTTATCGTCGTCATC | |
| Insertion | ID NO: 42) | CTTGTAATCTTCTGGCACC | |
| | | AGGA (SEQ ID NO: 43) | |
| FIG. 4b, e | GATTCCTGGTGC | TCTGCCCTCCCGTCACCC | CCTCTTCTTTGAC |
| DMNT1_FLAG- | CAGAAACA (SEQ | CTGTCTTATCGTCGTCATC | GCGGTTCTATCTA |
| Insertion_ | ID NO: 42) | CTTGTAATCTTCTGGCACC | GTTACGCGTTAAA |
| evopreQ1 | | AGGA (SEQ ID NO: 43) | CCAACTAGAAA |
| | | | (SEQ ID NO: 44) |
| FIG. 4b, e | GATTCCTGGTGC | TCTGCCCTCCCGTCACCC | CCTCTTCTGGGTC |
| DMNT1_FLAG- | CAGAAACA (SEQ | CTGTCTTATCGTCGTCATC | AGGAGCCCCCCC |
| Insertion_ | ID NO: 42) | CTTGTAATCTTCTGGCACC | CCTGAACCCAGG |
| mpknot | | AGGA (SEQ ID NO: 43) | ATAACCCTCAAAG |
| | | | TCGGGGGGCAAC |
| | | | CC (SEQ ID NO: 45) |
| FIG. 4c,f | GCATTTTCAGGA | TGTCTGAAGCCATCCCTTA | |
| RUNX_FLAG- | GGAAGCGA | TCGTCGTCATCCTTGTAAT | |
| Insertion | (SEQ ID NO: 46) | CCTTCCTCCTGAAAAT | |
| | | (SEQ ID NO: 47) | |
| FIG. 4c, f | GCATTTTCAGGA | TGTCTGAAGCCATCCCTTA | AACTCTCTTTGAC |
| RUNX_FLAG_ | GGAAGCGA | TCGTCGTCATCCTTGTAAT | GCGGTTCTATCTA |
| Insertion_ | (SEQ ID NO: 46) | CCTTCCTCCTGAAAAT | GTTACGCGTTAAA |
| evopreQ1 | | (SEQ ID NO: 47) | CCAACTAGAAA |
| | | | (SEQ ID NO: 48) |
| FIG. 4c, f | GCATTTTCAGGA | TGTCTGAAGCCATCCCTTA | AACTCTCTGGGTC |
| RUNX_FLAG- | GGAAGCGA | TCGTCGTCATCCTTGTAAT | AGGAGCCCCCCC |
| Insertion_ | (SEQ ID NO: 46) | CCTTCCTCCTGAAAAT | CCTGAACCCAGG |
| mpknot | | (SEQ ID NO: 47) | ATAACCCTCAAAG |
| | | | TCGGGGGGCAAC |
| | | | CC (SEQ ID NO: 49) |
| FIG. 4d, g | GATGTCTGCAG | AATGTGCCATCTGGAGCA | |
| VEGFA_FLAG- | GCCAGATGA | CTCACTTATCGTCGTCATC | |
| Insertion | (SEQ ID NO: 50) | CTTGTAATCTCTGGCCTGC | |
| | | AGA (SEQ ID NO: 51) | |
| FIG. 4d, g | GATGTCTGCAG | AATGTGCCATCTGGAGCA | ACAATCTCTTGAC |
| VEGFA_FLAG- | GCCAGATGA | CTCACTTATCGTCGTCATC | GCGGTTCTATCTA |
| Insertion_ | (SEQ ID NO: 50) | CTTGTAATCTCTGGCCTGC | GTTACGCGTTAAA |
| evopreQ1 | | AGA (SEQ ID NO: 51) | CCAACTAGAAA |
| | | | (SEQ ID NO: 52) |
| FIG. 4c, f | GATGTCTGCAG | AATGTGCCATCTGGAGCA | ACAATCTCGTCAG |
| VEGFA_FLAG- | GCCAGATGA | CTCACTTATCGTCGTCATC | GGTCAGGAGCCC |
| Insertion_ | SEQ ID NO: 50) | CTTGTAATCTCTGGCCTGC | CCCCCCTGCACC |
| mpknot | | AGA (SEQ ID NO: 51) | CAGGAAAACCCTC |
| | | | AAAGTCGGGGGG |
| | | | CAACCC (SEQ ID |
| | | | NO: 53) |

TABLE 3

| | | SEQ ID |
|---|---|---|
| Primer sequence | Sequence | NO: |
| FIG. 2d Fwd pegRNA | CCTTATTTTAACTTGCTATTTCTAGCTCTAAAAC | SEQ. |
| circular_check | | ID. NO: |
| | | 54 |
| FIG.2d Rev pegRNA | ATCAACTTGAAAAAGTGGCACCG | SEQ. |
| circular_check | | ID. NO: |
| | | 55 |
| FIG. 2d Fwd pegRNA | CGGTGCCACTTTTTCAAGTTGAT | SEQ. |
| linear_check | | ID. NO: |
| | | 56 |
| FIG. 2d Fwd pegRNA | GTTTTAGAGCTAGAAATAGCAAGTTAAAATAAG | SEQ. |
| linear_check | G | ID. NO: |
| | | 57 |
| FIG. 3a-f HEK3 PCR1 | ACACTCTTTCCCTACACGACGCTCTTCCGATCT | SEQ. |
| Fwd | ATGTGGGCTGCCTAGAAAGG | ID. NO: |
| | | 58 |
| FIG. 3a-f HEK3 PCR1 | GTGACTGGAGTTCAGACGTGTGCTCTTCCGAT | SEQ. |
| Rev | CTCCCAGCCAAACTTGTCAACC | ID. NO: |
| | | 59 |
| FIG. 4b, e | ACACTCTTTCCCTACACGACGCTCTTCCGATCT | SEQ. |
| DMNT1_FLAG- | CACAACAGCTTCATGTCAGCC | ID. NO: |
| Insertion Fwd | | 60 |
| FIG. 4b, e | TGGAGTTCAGACGTGTGCTCTTCCGATCTACGT | SEQ. |
| DMNT1_FLAG- | TAATGTTTCCTGATGGTCC | ID. NO: |
| Insertion Rev | | 61 |
| FIG. 4c,f RUNX_FLAG- | ACACTCTTTCCCTACACGACGCTCTTCCGATCT | SEQ. |
| Insertion Fwd | ACAAACAAGACAGGGAACTGG | ID. NO: |
| | | 62 |
| FIG. 4c,f RUNX_FLAG- | TGGAGTTCAGACGTGTGCTCTTCCGATCTCTAG | SEQ. |
| Insertion Rev | AGGGGTGAGGCTGAAAC | ID. NO: |
| | | 63 |
| FIG. 4d, g | ACACTCTTTCCCTACACGACGCTCTTCCGATCT | SEQ. |
| VEGFA_FLAG- | ACTTGGTGCCAAATTCTTCTCC | ID. NO: |
| Insertion Fwd | | 64 |
| FIG. 4d, g | TGGAGTTCAGACGTGTGCTCTTCCGATCTAAAG | SEQ. |
| VEGFA_FLAG- | AGGGAATGGGCTTTGGA | ID. NO: |
| Insertion Rev | | 65 |

mCherry_P2A_eGFP (mutant) Reporter sequence (SEQ. ID. NO: 66; Kozak sequence in bold, mCherry sequence in italics; P2A sequence in bold italics; eGFP sequence underlined with the targeted mutation in bold; and poly A sequence in bold italics underlined):

TCTGGCTAACTAGAGAACCCACTGCTTACTGGCTTATCGAAATTAATACGACTCACTATA
GGGAGACCCAAGCTGGCTAGCGTTTAAACGGGCCCTCTAGACTCGAGGCCACCATGG
TGAGCAAGGGCGAGGAGGACAACATGGCCATCATTAAGGAGTTCATGAGGTTCAAGGT
CCACATGGAGGGAAGCGTGAACGGCCACGAGTTCGAGATCGAGGGAGAGGGGGAGG
GCAGACCTTACGAGGGCACCCAGACCGCCAAGCTGAAGGTCACCAAGGGAGGCCCC
CTCCCCTTTGCTTGGGACATCCTGTCCCCCCAGTTCATGTACGGCAGCAAGGCCTATGT
CAAGCACCCCGCCGATATCCCCGACTACCTGAAGCTGTCCTTCCCCGAGGGCTTCAAG
TGGGAGAGGGTGATGAACTTCGAGGAGGGGGGGGTGGTCACCGTGACACAGGACTC
CAGCCTCCAGGACGGCGAGTTCATCTACAAGGTGAAGCTGAGGGGCACCAACTTCCC
TAGCGACGGCCCTGTCATGCAGAAGAAGACCATGGGATGGGAGGCCAGCTCCGAGAG
GATGTACCCCGAGGACGGCGCTCTGAAGGGCGAGATCAAGCAGAGGCTCAAGCTGAA
GGACGGCGGCCACTACGACGCCGAGGTGAAGACCACCTACAAAGCCAAGAAGCCCGT
GCAACTGCCCGGCGCCTACAACGTCAACATCAAGCTCGACATCACCTCCCACAACGAG
GACTACACCATCGTGGAGCAATACGAGAGGGCCGAGGGAAGACACAGCACAGGCGGC
ATGGACGAGCTGTACAAGGGCTCCGGCGCCACCACTTCAGCCTGCTCAAACAAGCC
GGCCATGTGGAGGAGAACCCTGGACCCGACTACAAGGACGACGACGACAAGGTGAG
CAAAGGCGAGGAGCTGTTCACCGGCGTGGTGCCTATCCTGGTCGAGCTGGACGGCGA
TGTGAACGGCCACAAGTTCAGCGTGTCCGGCGAAGGAGAGGGCGATGCCACCTATGG
CAAGCTGACCCTGAAGTTTATTTGCACTACTGGCAAACTGCCTGTTCCGTAGCCGACAC
TAGTGACGACGCTCTGCTATGGCGTCCAGTGCTTTTCAAGATACCCGGATCACATGAAA
CGGCATGACTTTTTCAAGAGTGCCATGCCCGAAGGTTATGTACAGGAAAGGACCATCTT
TTTCAAAGACGACGGCAACTACAAGACCAGGGCTGAGGTGAAGTTCGAGGGCGATACC
CTCGTGAACAGAATCGAGCTCAAGGGCATCGACTTCAAGGAGGACGGCAACATCCTCG
GCCACAAGCTGGAATACAATTACAACTCCCACAACGTCTATATCATGGCCGATAAGCAGA
AGAACGGCATTAAGGTCAATTTCAAGATCAGGCACAACATCGAGGACGGCAGCGTGCA
GCTGGCCGATCACTACCAGCAGAACACCCCCATCGGAGACGGACCTGTGCTGCTGCC
TGACAACCATTATCTGAGCACCCAGTCCGCCCTGTCCAAGGACCCTAACGAGAAGAGG
GACCACATGGTCCTCCTGGAGTTCGTGACCGCCGCTGGCATCACCCTGGGAATGGAC
GAGCTCTACAAATGAGGATCCGAGCTCGGTACCAAGCTTAAGTTTAAACCGCTGATCAG
CCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGCA
TCGCATTGTCTGAGTAGGTGTCATTCTATTCTGGGGGGTGGGGTGGGGCAGGACAGC
AAGGGGGAGGATTGGGAAGACAATAGCAGGCATGCTGGGGATGCGGTGGGCTCTAT
GG

pUCIDT_AmpR_pU6 sequence (SEQ. ID. NO: 67; U6 promoter sequence in bold italics and underlined; cpegRNA sequence in italics; ColE1/pMB1/pBR322/pUC sequence in bold and underlined; AmpR promotor sequence underlined; and AmpR sequence in bold):

TCGCGCGTTTCGGTGATGACGGTGAAAACCTCTGACACATGCAGCTCCCGGAGACGG
TCACAGCTTGTCTGTAAGCGGATGCCGGGAGCAGACAAGCCCGTCAGGGCGCGTCAG
CGGGTGTTGGCGGGTGTCGGGGCTGGCTTAACTATGCGGCATCAGAGCAGATTGTACT
GAGAGTGCACCAAATGCGGTGTGAAATACCGCACAGATGCGTAAGGAGAAAATACCGC
ATCAGGCGCCATTCGCCATTCAGGCTGCGCAACTGTTGGGAAGGGCGATCGGTGCGG
GCCTCATCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGCAACGC
GATGACGATGGATAGCGATTCATCGATGAGCTGACCCGATCGCCGCCGCCGGAGGGTT
GCGTTTGAGACGGGCGACAGATGAGGGCCTATTTCCCATGATTCCTTCATATTTGCATA
TACGATACAAGGCTGTTAGAGAGATAATTAGAATTAATTTGACTGTAAACACAAAGATA
TTAGTACAAAATACGTGACGTAGAAAGTAATAATTTCTTGGGTAGTTTGCAGTTTTAAA
ATTATGTTTTAAAATGGACTATCATATGCTTACCGTAACTTGAAAGTATTTCGATTTCTTG
GCTTTATATATCTTGTGGAAAGGACGAAACACCGCCATCAGTCGCCGGTCCCAAGCCC
GGATAAAATGGGAGGGGGCGGGAAACCGCCTAACCATGCCGACTGATGGCAGAAAAA
AAAAAGGCCCAGACTGAGCACGTGAGTTTTAGAGCTAGAAATAGCAAGTTAAAATAAGG
CTAGTCCGTTATCAACTTGAAAAAGTGGCACCGAGTCGGTGCTCTGCCATTACGTGCTC
AGTCTGAAAAAAAAAGCTGCCATCAGTCGGCGTGGACTGTAGAACACTGCCAATGCCG
GTCCCAAGCCCGGATAAAAGTGGAGGGTACAGTCCACGCTTTTTTTCATGGTCATAGCT
GTTTCCTTTTTTTCGTACTGAGTCGCCCAGTCTCAGAATCAGTTCTGGACCAGCGAGCT
GTGCTGCGACTCGTGGCGTAATCATGGTCATAGCTGTTTCCTGTGTGAAATTGTTATCC
GCTCACAATTCCACACAACATACGAGCCGGAAGCATAAAGTGTAAAGCCTGGGGTGCC
TAATGAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGGCGGTTT
GCGTATTGGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGTCGCTTACC
GGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAGC
GAGGTATGTAGGCGGTGCTACAGAGTTCTTGAAGTGGTGGCCTAACTACGGCTACAC
TAGAAGAACAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTTTTTTGTT
GCAAGCAGCAGATTACGCGCAGAAAAAAAGGATCTCAAGAAGATCCTTTGATCTTTTC
TACGGGGTCTGACGCTCAGTGGAACGAAAACTCACGTTAAGGGATTTTGGTCATGAGAT
TATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAAC
TACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGC
GCAGAAGTGGTCCTGCAACTTTATCCGCCTCCATCCAGTGTATTAATTGTTGCCGGGA
AGCTAGAGTAAGTAGTTCGCCAGTTAATAGTTTGCGCAACGTTGTTGCCATTGCTACA
GGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTG
AGTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCATT
GGAAAACGTTCTTCGGGGCGAAAACTCTCAAGGATOTTACCGOTGTTGAGATCCAGT
TCGATGTAACCCACTCGTGCACCCAACTGATCTTCAGCATCTTTTACTTTCACCAGCGT
TTCTGGGTGAGCAAAAACAGGAAGGCAAAATGCCGCAAAAAAGGGAATAAGGGCGA
CACGGAAATGTTGAATACTCATATGCCGCAAAAAAGGGAATAAGGGCGACACGGAAAT
GTTGAATACTCATACTCTACCTTTTTCAATATTATTGAAGCATTTATCAGGGTTATTGTCTC
ATGAGCGGATACATATTTGAATGTATTTAGAAAAATAAACAAATAGGGGTTCCGCGCACAT
TTCCCCGAAAAGTGCCACCTGACGTCTAAGAAACCATTATTATCATGACATTAACCTATAA
AAATAGGCGTATCACGAGGCCCTTTCGTC

To develop a high-throughput method for screening of cpegRNA library against a panel of known disease mutations, the inventors are making use of a pegRNA library transduction method (13) and had preliminarily chosen 500 pegRNAs against the subset of disease targets (Table 4). Integration of each pegRNA construct into genome of the cells is coupled for functional verification using a corresponding target mutation located downstream (FIG. 4A-4G). As such, we can transiently express PEmax and access for genomic modification using one-step PCR and NGS method. This method can provide us with a genome-wide capability to assess for all the possible disease mutations. In doing so, the inventors can iteratively optimize different pegRNA parameters (Spacer, PBS, RTT) and can further modify the Tornado backbone to access for better efficiency of the platform against different cell-types. As a result, the inventors are developing an attention-based bidirectional recurrent neural network to develop an advanced in sillico design tool for the circularized guided system.

In parallel, the inventors are formulating LNP to deliver the RNA components of cpegRNA in primary patient cells. To show proof-of-concept ex viva, we are working on delivering in vitro transcribed cpegRNA and PEmax mRNA (5′ cap, pseudouridine and polyA) in primary cells of a patient with Tay-Sachs disease to rescue the insertion (TATC) within the HEXA gene. To achieve this aim, we are initially making use of a SORT¹³ LNP formulation that can be readily made using the described pipetting or vortexing methodology.

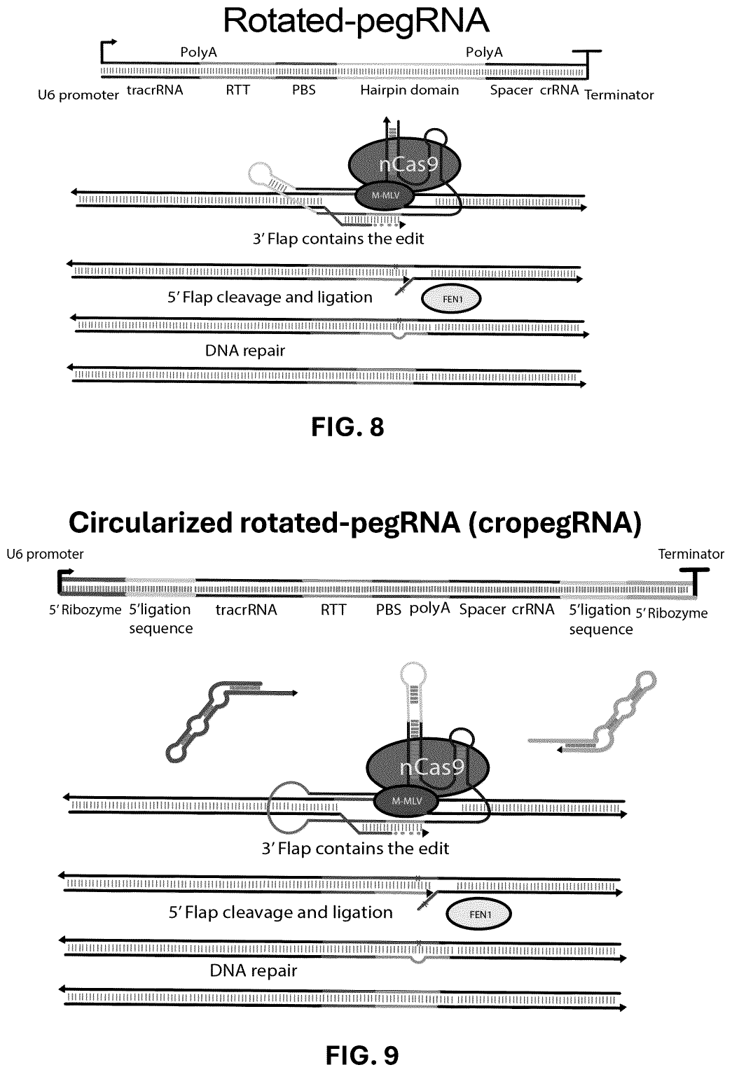

Alternative strategy using rotated pegRNAs (rpegRNAs): In addition to the use of circularized guide RNA methodologies that are discussed above, further use of the approach describe herein can be fine-tuned using a pegRNA transcript that is flanked via tracrRNA and crRNA domains (FIG. 8). In this strategy, the guide RNA is not circularized but the ends of the transcript can be protected from nucleases by the CRISPR/Cas enzyme. The portion of the pegRNA containing the spacer and RTT can be kept the same as typical circularized pegRNAs. For instance, the start of the transcript can be positioned either within tetraloop as well as stem loops 1, 2 and 3 (aka ST1,2 and 3 loops) of the Cas9 gRNA. Further extension of this domain can be iteratively optimized with various levels of complementarity to enhance the functionality of CRISPR platform. For example, the use of ST2 loop had been combined with MS2 and PP7 aptamer binding domains for various recruitment strategies. The use of this system can make use of additional hairpin domain that can retain PBS and crRNA functionality as seen in the existing circularized system.

Possible Variations: The circularization of the gRNA or gDNA can be used toward any CRISPR-like technology that is programmable by nature (or direct-evolved) and can be applied for in vitro (cell-free diagnostic), ex vivo and in vivo (therapeutics) applications. For example, the use of circular gRNA can be beneficial in therapeutic applications that leverages Base-Editors (BEs) (18, 19), TwinPE (9), PASTE (10), TJ-PE (11), PrimeDel (12), PEDAR (13), dual-pegRNAs (14), HOPE (15), GRAND (16) and Bi-PE (17) in addition to REPAIR (26) and RESCUE (27) that are RNA base-editing platforms. Moreover, the use of circular gRNA can be beneficial for use in diagnostic applications, for example in SHERLOCK (22, 23) and DETECTR (24) platforms. In each case, the circularization of the gRNA can provide for long-lasting and programmable functionality of the RNP which can improve the diagnostic and therapeutic applications via enhancing the guided stability for detection and modification purposes.

The use of circularized gRNA can also be leveraged toward the recently characterized mammalian CRISPR-like platform OMEGA (31) (aka HERMES (32)) which encodes for a RNA-guided IsrB nickase that is part of the IS200/IS605 super family of transposons. Broadly, the use of IsrB within therapeutic applications can be beneficial due to the humanized nature of the protein which can omit immunogenicity issues. For mammalian genome engineering, currently OMEGA requires the overexpression of both the IsrB protein and gRNA system (31). We envision that the use of endogenous IsrB can be reprogrammed using our circularized gRNA system. Additionally, we can make use of the IsrB within PE application via supplying a pegRNA that couples a reverse transcriptase domain to the IsrB nickase.

Additional means to leverage the circularized gRNA or gDNA strategy can make use of the argonaute proteins, which are expressed constitutively in mammalian cells and are required for processing of non-coding RNA (microRNA and (si)RNA) to induce programmable RNA silencing (33). In addition, the argonautes from prokaryotic ancestor function to use ssDNA or ssRNA guides to induce nicking against ssDNA or dsDNA substrates (34). As such, we can also utilize argonaute nickases in fusion with RT and helicase domains for coupling with our circularized guided system within PE

POC Diagnostic applications: The SHERLOCK (28, 29) and DETECTR (30) platform make use of gRNA to respectively detect for the desired RNA and DNA targets using the Cas13 and Cas12 trans-cleavage nuclease activity that are made against ssRNA and ssDNA reporter. The use of SHERLOCK and DETECTR for POC settings require for a shelf-stable product, however over time the gRNA can be degraded via 5′ and 3′ exonucleases. The use of SHERLOCK and DETECTR therefore can benefit from circularized gRNA component for development of a shelf-stable product. The use of circularized gRNA may also improve the Cas12 and Cas13 multi-turnover trans-nuclease activity and develop for a better sensitivity of detection against their targets.

Portable targeted sequencing applications: Due to the large size of the human genome, the existing whole genome sequencing methods are both laborious and cost-effective and often time these strategies can not infer for mutations that account for less than 5% of the target alleles as seen in liquid biopsy samples. To develop for a targeted sequencing approach with higher coverage of reads, the Oxford Nanopore Technology (ONT) had been coupled with Cas9 targeted cleavage approaches for appending of sequencing adaptors to the desired genomic loci (35). In this method, multiple gRNAs may be required to cleave the desired genomic loci in question. We envision that the use of circularized gRNA in this context can induce for a higher cleavage rate and therefore provide for higher coverage of sequencing reads eliminating the need for multiple gRNAs.

Therapeutic applications: Prior to development of PE, the Base-Editors (BEs) were developed to address for a subset of genomic base-substitutions (24, 25). The BE leverages a fusion between dead or nicking Cas9 and a (directed evolution derived) Adenine/Cytosine deaminase domain. The use of circularized sgRNA within BEs can improve genomic modification and resist against MMR reversion. Similar to the PE3 platform, the BE3 platform require secondary sgRNA nicking of the unedited strand to obtain adequate efficiency. Since the secondary sgRNA nicking of the unedited strand can result into DDBs and indel formation, alternative strategies to increase BE efficiency are desired. We envision that the use of circularized gRNA for BEs can improve its efficiency.

For large size genomic insertion applications the TwinPE has coupled the use of dual pegRNAs for targeted addition of the integrase landings sites (attB or attP) and DNA integration using Bxb1 integrase (9). Although the TwinPE can provide for gene-size insertion, its efficiency remains to be very low. We envision that the use of circularized pegRNA can improve TwinPE for large size DNA integration.

Alternative method for large size genomic insertion is PASTE, which requires nCas9 fusion to MMLV RT and Bxb1 integrase coupled with atgRNA (pegRNA that encodes for attB within the RTT domain) and a nicking sgRNA that is designed on the unedited strand (10). PASTE has a superior performance to the TwinPE platform as it provides for more than 20-fold increase in the insertion efficiency. To improve PASTE insertion efficiency, the use of described circularized gRNA strategy can be applied to both the atgRNA as well as the nicking sgRNA.

Erik J. Sontheimer et al. have also developed Template-Jumping (TJ) PE to drive shorter targeted genomic insertion that ranges between 200-500 bp in length. The TJ-PE platform requires a pegRNA and nicking sgRNA that are designed ˜90 bp apart in the opposing strands. In the TJ-PE system, the the natively synthesized 3′ flap domain from pegRNA is made cognate to the 3′ flap domain that results from the adjacent sgRNA nicking event and provides for template-jumping event and targeted synthesis. The use of TJ-PE can also benefit from circularization of the pegRNA as well as the nicking sgRNA components.

To develop for enabled CRISPR platforms that induces base-modification at transcriptomic level, Feng Zhang et al. had created REPAIR (RNA-Editing for Programmable A to I Replacement) and RESCUE (RNA Editing for Specific C to U Exchange) (26, 27). The REPAIR and RESCUE both make use of fusion between dCas13b and ADAR2 adenosine deaminase domains that underwent rational protein engineering for site-directed transcriptomic base-editing. We envision that the use of REPAIR and RESCUE can be improved via circularization of the gRNA component. This can provide for enhanced transcriptomic base modification where further stability of the circularized gRNA within the RNP complex can derive more number of edits per transcript.

RNA circularization and RNA modification methods: Other means of RNA circularization methods can be utilized to enhance the gRNA stability. In addition to P1 and P3 ribozymes used in Tornado, other natural or evolved ribozyme domains can be used to enhance the RtcB substrate formation. Other endogenous or exogenous ligases can also be utilized for the gRNA circularization. The exogenous ligase can be alternatively supplied as an in-frame fusion, or via a self-cleaving peptide to the PE, BEs, PASTE, RESCUE and REPAIR molecular machineries. To increase the efficiency of the gRNA self-circularization within Tornado, the ligation domains can undergo further iterative screening. These set of iterations can provide for various levels of self-complementarity within the 5′ and 3′ end of the RNA payload to physically join the two ends and enforce the self-ligation event. To omit secondary structure barrier within the gRNA domains, the 5′ and 3′ end of the gRNA can be flanked with polyA (existing strategy), polyC, polyG, or any domains with neutral secondary. The use of polyT can be problematic due to transcriptional termination from the U6 promoter but can be considered for chemical synthesis applications. gRNA self-circularization can also be accomplished through RNA self-splicing systems. For instance, self-splicing introns have been used to form circularized mRNA molecules (43). Other such systems, either identified from nature or those that have been engineered, are likely to be a valuable methods for gRNA circularization without the use of Tornado or the need for enzymes like RtcB.

For RNA delivery strategies such as LNP or nucleofection, the use of modified nucleotides within the CRISPR mRNA, pegRNA and sgRNA can further enhance the RNA half-life and overcome immunogenicity issues. For example, the use of pseudo-uridine is known to stabilize mRNA and overcome undesired immune response. Making use of 5′ cap and 3′ polyA within the CRISPR mRNA can also obviate immune related problems and improve the translation efficiency in the mammalian context. The use chemically synthesized pegRNA with three 2′OMe and three phosphorothioate base pairs on RNA ends is currently the state-of-the art for PE nucleofection in primary cells.

Broadening the genomic target and insertion: To broaden the use of PE platform, we can leverage PE toward insertion and/or modification of endogenous loci that includes but not limited to promoters, enhancers and epigenetic elements including CpG islands. The insertion of integrase recognition domains particularly in genomic loci that are known to safely harbor mutations can be developed for universal cell-lines that bear landing sites for DNA donation. The cargoes can encode for coding and non coding RNAs. In the case of coding elements, the desired ORFs can encode for proteins that have a broad set applications such as DNA binding proteins, RNA binding proteins or epigenetic modulators that can be used to alter gene expression at transcriptional and translational levels. Not to mention, the PE can be used to insert ORFs into an existing protein coding genes like housekeeping genes that are being constitutively expressed, and this can be done by using a P2A self-cleaving peptide that drives the co-expression of the two or more proteins using the housekeeping promoter. Alternatively, the ORFs can be fused downstream of the genes that are only expressed in response to a particular condition, for example over expression of an oncogene(s). This can develop as a conditional means to express a particular payload that only gets expressed in cancer cell types that have an unregulated oncogene.

The PE enabled site-directed insertion of non-coding RNA can be leveraged toward a broad set of elements like microRNAs and gRNAs that can be programmed with CRISPR or (endogenous) ADAR systems. The site directed encoding of non-coding and coding RNAs can be coupled with ribozymes (P1 and P3) and self-cleaving peptides (P2A and T2A) to encode for multiple RNAs and proteins, and their combination thereof, for example programmable RNPs with broad set of functionalities. The expression of coding and non-coding RNAs can be derived by endogenous or exogenous promoters. The related promoters can be programmed for activation in a conditional environment, for example using a directed heat or light responsive promoters. Alternatively, the promoters can be activated via extra-cellular environment, for example, the use of SynNotch (13) or SynZiFTR (38) receptors that can release a specific TF or Zinc-finger TF fusion that can regulate the expression of a particular payload using a promoter element that gets conditionally activated in response to an antigen recognition on the extra-cellular domain of the CART cell.

The use of the platform can have profound impact when leveraged toward plant engineering applications. Similar to mammalian context, the integration of gene-sized cargo within plants can be developed for expression of biosynthetic pathways that can give rise to chemicals with importance in bio-energy and commodities. However, since plants do not possess the endogenous RtcB enzyme, the circularization of the pegRNA can be derived in vitro for delivery in plant cells. Alternatively, the engineering of PE system that co-expresses RtcB ligase in fusion can be used for plant engineering applications or an RtcB-free circularization scheme could be used as described above.

Alternative Methods for Circularization: To enable gRNA circularization, we made use of Tornado that consists of a flanking P1 and P3 Twister ribozymes that undergo auto-cleavage to generate the respective substrates for the RtcB ligase (5′ hydroxyl and a 2′,3′-cyclic) and derive the self-ligation event (4,5). The Tornado system also contains flanking inner domains that bear partial complementarity and may enforce the proximity of the termini to assist with the self-ligation event. To couple the use of Tornado in our pegRNA system, we had additionally appended flanking polyA positioned upstream of the 5′ spacer and downstream of the 3′ PBS domains. This was done to omit possible secondary structure barriers that may interfere with the pegRNA functionality.

The use of our technology however can be generalizable to different methods of RNA circularization that includes ribozyme and splicing based platforms. Ribozyme based domains are natural and (direct) evolved parts that are originally found in the non-coding RNAs. Specifically Twister ribozyme belong to a self-cleaving family of ribozymes with 2,700 known consensus (6). Alternatively self-splicing domains that belong to group I introns can also serve to derive the self-circularization in vitro (7,8).

Other embodiments: The technology described herein can be used for in vitro, ex vivo and in vivo applications once coupled with the state-of-the-art CRISPR enabled technologies. In the context of ex vivo engineering, the primary cells from patients with genetic diseases can be reverted back to the wild-type sequence for further expansion in cell culture and delivered to the patient for therapeutic purposes. In addition to the 12 types of base-modification, the technology can be leveraged toward deletion and insertion within precise sites in the genome, which together can address for ˜90% known human genetic abnormalities. The use of the PE has also been leveraged toward integration of gene-sized cargo. There had been several approaches that can embed gene-sized cargo within precise loci in the genome, namely TwinPE, (9) PASTE, (10) and TJ-PE, (11) and other dual pegRNA approaches that include PrimeDel (12), PEDAR (13), dual-pegRNAs (14), HOPE (15), GRAND (16) and Bi-PE (17).

Another important advance within ex vivo engineering can be leveraged toward CAR-T engineering (18). The isolation of the primary T cells from patient can be followed by integration of the DNA payload at the precise genomic loci that is known to harbor safety. For these application integrase (e.g. attP or attB from Bxb1 integrase) recognition sequence can be encoded in the target loci of interest using the described circularized pegRNA system. This then can provide for site specific integration of CAR domains and larger gene size circuitry using a DNA donor that contains flanking integration motifs. For these set of applications, larger DNA payloads that are more than one gene can be fine-tuned for site-directed genomic integration or multiplexed integration at different loci. For example, the integration of the SynNotch receptors and its respective downstream gene can be used for DNA cargo integration (19).

Other applications of CART cell engineering can include development of allogenic CART cells that can be used as an “off-the-shelf” therapy and could be made tailored based on the patient's need (20). The existing CRISPR HDR strategies for allogenic CART cell engineering have low efficiency and often time result into heterogeneous indel formation at the targeted loci. As such, the existing methods to develop allogenic CART cells are challenging given the short life of primary T-cells.

For the use of the technology described herein for in vivo applications, one can leverage the split based constructs descried herein within an AAV payload. In addition, LNP strategies can be used to deliver the system upon RNA encapsulation of the cargo. The use of LNP can also be fine-tuned for RNA delivery that harbors tissue-specific tropism and can be achieved using several LNP formulations (21). The LNP strategy can additionally provide for non-toxic mean to engineer primary cells ex vivo and provide for in vivo delivery in clinical trials. The use of the technology described herein can additionally provide for multiplexed genomic modification to address for multiple co-occuring genetic abnormalities that may arise in cancer related conditions that disrupts DNA repair pathway(s).

To develop a safer delivery strategy that harbors lower off-targets, further use of the technology described herein for in vivo applications can leverage eVLPs for transient delivery of the ribonucleoprotein (RNP) (22). This technology was more recently optimized against the PE system (40) where the RNP was fused to the MLV-gag domain via a self-cleaving peptide to enable transient endosomal release of the complex. In this method, further appending of MS2 to the pegRNA and MCP on the viral particle had shown to enhance the RNP delivery per particle. Further use of the p3-p4 coiled-coil pairs was also leveraged to couple Cas9 nickase to the RT domain. In parallel, seminal work by Feng Zhang group had shown the use of antibody within the VSV-G viral surface domain to develop for an eVLP with cell-classifying capability (23). In addition, other efforts by Jennifer Doudna group developed Cas9-packaging enveloped delivery vehicles (Cas9-EDVs) that leverages mutant form of VSV-G that requires ScFv for multiplexed and orthogonal cell classification (41).

### REFERENCES

1. Nelson, J. W. et al. Engineered pegRNAs improve prime editing efficiency. Nat Biotechnol 40, 402-410 (2022).
2. Liu, B. et al. A split prime editor with untethered reverse transcriptase and circular RNA template. Nat Biotechnol 40, 1388-1393 (2022).
3. Wesselhoeft, R. A. et al. RNA Circularization Diminishes Immunogenicity and Can Extend Translation Duration In Vivo. Molecular Cell 74, 508-520.e4 (2019).
4. Litke, J. L. & Jaffrey, S. R. Highly efficient expression of circular RNA aptamers in cells using autocatalytic transcripts. Nat Biotechnol 37, 667-675 (2019).
5. Jaffrey, S. R. & LITKE, J. L. Rna molecules, methods of producing circular rna, and treatment methods. (2021).
6. Roth, A. et al. A widespread self-cleaving ribozyme class is revealed by bioinformatics. Nat Chem Biol 10, 56-60 (2014).
7. Wesselhoeft, R. A., Kowalski, P. S. & Anderson, D. G. Engineering circular RNA for potent and stable translation in eukaryotic cells. Nat Commun 9, 2629 (2018).
8. Liu, L. et al. Circular Guide RNA for Improved Stability and CRISPR-Cas9 Editing Efficiency in Vitro and in Bacteria. ACS Synth. Biol. 12, 350-359 (2023).
9. Anzalone, A. V. et al. Programmable deletion, replacement, integration and inversion of large DNA sequences with twin prime editing. Nat Biotechnol 40, 731-740 (2022).
10. Yarnall, M. T. N. et al. Drag-and-drop genome insertion of large sequences without double-strand DNA cleavage using CRISPR-directed integrases. Nat Biotechnol 41, 500-512 (2023).
11. Zheng, C. et al. Template-jumping prime editing enables large insertion and exon rewriting in vivo. Nat Commun 14, 3369 (2023).
12. Choi, J. et al. Precise genomic deletions using paired prime editing. Nat. Biotechnol. 40, 218-226 (2022).
13. Jiang, T., Zhang, X.-O., Weng, Z. & Xue, W. Deletion and replacement of long genomic sequences using prime editing. Nat. Biotechnol. 40, 227-234 (2022).
14. Lin, Q. et al. High-efficiency prime editing with optimized, paired pegRNAs in plants. Nat. Biotechnol. 39,923-927 (2021).
15. Zhuang, Y. et al. Increasing the efficiency and precision of prime editing with guide RNA pairs. Nat. Chem. Biol. 18, 29-37 (2022).
16. Wang, J. Efficient targeted insertion of large DNA fragments without DNA donors. Nat. Methods 19, 25 (2022).
17. Tao, R. et al. Bi-PE: bi-directional priming improves CRISPR/Cas9 prime editing in mammalian cells. Nucleic Acids Res. 50, 6423-6434 (2022).
18. Dimitri, A., Herbst, F. & Fraietta, J. A. Engineering the next-generation of CAR T-cells with CRISPR-Cas9 gene editing. Molecular Cancer 21, 78 (2022).
19. Morsut, L. et al. Engineering Customized Cell Sensing and Response Behaviors Using Synthetic Notch Receptors. Cell 164, 780-791 (2016).
20. Sheridan, C. Off-the-shelf, gene-edited CAR-T cells forge ahead, despite safety scare. Nature Biotechnology 40, 5-8 (2021).
21. Wang, X. et al. Preparation of selective organ-targeting (SORT) lipid nanoparticles (LNPs) using multiple technical methods for tissue-specific mRNA delivery. Nat Protoc 18, 265-291 (2023).
22. Banskota, S. et al. Engineered virus-like particles for efficient in vivo delivery of therapeutic proteins. Cell 185, 250-265.e16 (2022).
23. Strebinger, D. et al. Cell type-specific delivery by modular envelope design. Nat Commun 14, 5141 (2023).
24. Gaudelli, N. M. et al. Programmable base editing of A·T to G·C in genomic DNA without DNA cleavage. Nature 551, 464-471 (2017).
25. Neugebauer, M. E. et al. Evolution of an adenine base editor into a small, efficient cytosine base editor with low off-target activity. Nat Biotechnol 41, 673-685 (2023).
26. Cox, D. B. T. et al. RNA editing with CRISPR-Cas13. Science 358, 1019-1027 (2017).
27. Abudayyeh, O. O. et al. A cytosine deaminase for programmable single-base RNA editing. Science 365, 382-386 (2019).
28. Gootenberg, J. S. et al. Nucleic acid detection with CRISPR-Cas13a/C2c2. Science 356, 438-442 (2017).
29. Gootenberg, J. S. et al. Multiplexed and portable nucleic acid detection platform with Cas13, Cas12a, and Csm6. Science 360, 439-444 (2018).
30. Broughton, J. P. et al. CRISPR-Cas12-based detection of SARS-CoV-2. Nat Biotechnol 38, 870-874 (2020).
31. Hirano, S. et al. Structure of the OMEGA nickase IsrB in complex with ωRNA and target DNA. Nature 610, 575-581 (2022).
32. Jiang, K. et al. Programmable RNA-guided endonucleases are widespread in eukaryotes and their viruses. 2023.06.13.544871 Preprint at https://doi.org/10.1101/2023.06.13.544871 (2023).
33. Hutvagner, G. & Simard, M. J. Argonaute proteins: key players in RNA silencing. Nat Rev Mol Cell Biol 9, 22-32 (2008).
34. Hegge, J. W., Swarts, D. C. & van der Oost, J. Prokaryotic Argonaute proteins: novel genome-editing tools?Nat Rev Microbiol 16, 5-11 (2018).
35. Gilpatrick, T. et al. Targeted nanopore sequencing with Cas9-guided adapter ligation. Nat Biotechnol 38, 433-438 (2020).
36. Liang, S.-Q. et al. Genome-wide profiling of prime editor off-target sites in vitro and in vivo using PE-tag. Nat Methods 20, 898-907 (2023).
37. Koeppel, J. et al. Prediction of prime editing insertion efficiencies using sequence features and DNA repair determinants. Nat Biotechnol 1-11 (2023) doi:10.1038/s41587-023-01678-y.
38. Li, H.-S. et al. Multidimensional control of therapeutic human cell function with synthetic gene circuits. Science 378, 1227-1234 (2022).
39. Liang, R. et al. Prime editing using CRISPR-Cas12a and circular RNAs in human cells. Nat Biotechnol 1-9 (2024) doi:10.1038/s41587-023-02095-x.
40. An, M. et al. Engineered virus-like particles for transient delivery of prime editor ribonucleoprotein complexes in vivo. Nat Biotechnol 1-12 (2024) doi:10.1038/s41587-023-02078-y.
41. Hamilton, J. R. et al. In vivo human T cell engineering with enveloped delivery vehicles. Nat Biotechnol 1-9 (2024) doi:10.1038/s41587-023-02085-z.
42. Mathis, N. et al. Predicting prime editing efficiency and product purity by deep learning. Nat Biotechnol 41, 1151-1159 (2023).
43. Wesselhoeft, R. A., Kowalski, P. S. & Anderson, D. G. Engineering circular RNA for potent and stable translation in eukaryotic cells. Nat Commun 9, 2629 (2018). https://doi.org/10.1038/s41467-018-05096-6

TABLE 4
Lentiviral based cpegRNA screen against a panel of known disease mutations was
adopted⁴³ which varies PBS and/or RTT length against the given genetic mutation sequence
to screen for the most suitable cpegRNA against the desired disease (FIG. 7). The IDT oligo
pool sample sequence below is used for amplification and cloning into lentiviral plasmid that
bears U6 promoter, 5′ Ribozyme domain, polyA sequence, NsiI and BlpI restriction domains.
Sample annotation: 5′ Primer and NsiI Restriction Sequence-lowercase italics; spacer-
underline, sgRNA Scaffold-UPPERCASE ITALICS; RTT-bold; PBS-underline italics;
PolyA; 3′Ribozyme-bold underline, polyT, Target mutation sequence-bold underline
italics; and 3′ primer BiPI Restriction sequence-lowercase italics

| | | SEQ ID |
|---|---|---|
| | Sequence | NO: |
| Phenylketonuria; | ggccaaagcatgcatGGGGTCGTAGCGAACTGAGAGTTTCAGAGCT | 68 |
| not | ATGCTGGAAACAGCATAGCAAGTTGAAATAAGGCTAGTCCGTT | |
| provided | ATCAACTTGAAAAAGTGGCACCGAGTCGGTGCCCTCGGCCCT | |
| | TCTCAGTTCGCTACGAAAAAAAAAAGCTGCCATCAGTCGGCGT | |
| | GGACTGTAGAACACTGCCAATGCCGGTCCCAAGCCCGGATA | |
| | AAAGTGGAGGGTACAGTCCACGCTTTTTTTGGTTTTGGTCTTA | |
| | GGAACTTTGCTGCCACAATACCTTGGCCCTTCTCAGTTCGCT | |
| | ACGACCCATACACCCAAAGGTCCgctaagcagcttggcgtaactagat | |
| | ct | |
| Phenylketonuria; | ggccaaagcatgcatGGGGTCGTAGCGAACTGAGAGTTTCAGAGCT | 69 |
| not | ATGCTGGAAACAGCATAGCAAGTTGAAATAAGGCTAGTCCGTT | |
| provided | ATCAACTTGAAAAAGTGGCACCGAGTCGGTGCAATACCTCGG | |
| | CCCTTCTCAGTTCGCTACGAAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTTGGTCTTAG | |
| | GAACTTTGCTGCCACAATACCTTGGCCCTTCTCAGTTCGCTAC | |
| | GACCCATACACCCAACAGGCAgctaagcAGCTTGGCGTAACTAG | |
| | ATCT | |
| Phenylketonuria; | ggccaaagcatgcatGGGGTCGTAGCGAACTGAGAGTTTCAGAGCT | 70 |
| not | ATGCTGGAAACAGCATAGCAAGTTGAAATAAGGCTAGTCCGTT | |
| provided | ATCAACTTGAAAAAGTGGCACCGAGTCGGTGCGCTGCCACAA | |
| | TACCTCGGCCCTTCTCAGTTCGCTACGAAAAAAAAAAGCTGCC | |
| | ATCAGTCGGCGTGGACTGTAGAACACTGCCAATGCCGGTCCC | |
| | AAGCCCGGATAAAAGTGGAGGGTACAGTCCACGCTTTTTTTAG | |
| | GAACTTTGCTGCCACAATACCTTGGCCCTTCTCAGTTCGCTAC | |
| | GACCCATACACCCAAGAAGGTgctaagcAGCTTGGCGTAACTAG | |
| | ATCT | |
| Phenylketonuria; | ggccaaagcatgcatGGGTCGTAGCGAACTGAGAAGTTTCAGAGCT | 71 |
| not | ATGCTGGAAACAGCATAGCAAGTTGAAATAAGGCTAGTCCGTT | |
| provided | ATCAACTTGAAAAAGTGGCACCGAGTCGGTGCCCTCGGCCCT | |
| | TCTCAGTTCGCTACGAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGTGGTTTTGGTCTTA | |
| | GGAACTTTGCTGCCACAATACCTTGGCCCTTCTCAGTTCGCTA | |
| | CGACCCATACACCCAGCATTGgctaagcAGCTTGGCGTAACTAG | |
| | ATCT | |
| Phenylketonuria; | ggccaaagcatgcatGGGTCGTAGCGAACTGAGAAGTTTCAGAGCT | 72 |
| not | ATGCTGGAAACAGCATAGCAAGTTGAAATAAGGCTAGTCCGTT | |
| provided | ATCAACTTGAAAAAGTGGCACCGAGTCGGTGCAATACCTCGG | |
| | CCCTTCTCAGTTCGCTACGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTTTTTGGTCTTAG | |
| | GAACTTTGCTGCCACAATACCTTGGCCCTTCTCAGTTCGCTAC | |
| | GACCCATACACCCACTTAACgctaagcAGCTTGGCGTAACTAGAT | |
| | CT | |
| Phenylketonuria; | ggccaaagcatgcatGGGTCGTAGCGAACTGAGAAGTTTCAGAGCT | 73 |
| not | ATGCTGGAAACAGCATAGCAAGTTGAAATAAGGCTAGTCCGTT | |
| provided | ATCAACTTGAAAAAGTGGCACCGAGTCGGTGCCACAATACCTC | |
| | GGCCCTTCTCAGTTCGCTACGAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTTGGTCTTAG | |
| | GAACTTTGCTGCCACAATACCTTGGCCCTTCTCAGTTCGCTAC | |
| | GACCCATACACCCAGCGTAAgctaagcAGCTTGGCGTAACTAGAT | |
| | CT | |
| Phenylketonuria; | ggccaaagcatgcatGGGTCGTAGCGAACTGAGAAGTTTCAGAGCT | 74 |
| not | ATGCTGGAAACAGCATAGCAAGTTGAAATAAGGCTAGTCCGTT | |
| provided | ATCAACTTGAAAAAGTGGCACCGAGTCGGTGCGCTGCCACAA | |
| | TACCTCGGCCCTTCTCAGTTCGCTACGAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTTTA | |
| | GGAACTTTGCTGCCACAATACCTTGGCCCTTCTCAGTTCGCTA | |
| | CGACCCATACACCCATTCTAAgctaagcAGCTTGGCGTAACTAGA | |
| | TCT | |
| Phenylketonuria; | ggccaaagcatgcatGAGCGAACTGAGAAGGGCCAGTTTCAGAGCT | 75 |
| not | ATGCTGGAAACAGCATAGCAAGTTGAAATAAGGCTAGTCCGTT | |
| provided | ATCAACTTGAAAAAGTGGCACCGAGTCGGTGCCCTCGGCCCT | |
| | TCTCAGTTCAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTTCACTCAAGCCTGTGGTTTTG | |
| | GTCTTAGGAACTTTGCTGCCACAATACCTTGGCCCTTCTCAGT | |
| | TCGCTACGACCCATAACTAGCgctaagcAGCTTGGCGTAACTAGA | |
| | TCT | |
| Phenylketonuria; | ggccaaagcatgcatGAGCGAACTGAGAAGGGCCAGTTTCAGAGCT | 76 |
| not | ATGCTGGAAACAGCATAGCAAGTTGAAATAAGGCTAGTCCGTT | |
| provided | ATCAACTTGAAAAAGTGGCACCGAGTCGGTGCAATACCTCGG | |
| | CCCTTCTCAGTTCAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTCAAGCCTGTGGTTTTG | |
| | GTCTTAGGAACTTTGCTGCCACAATACCTTGGCCCTTCTCAGT | |
| | TCGCTACGACCCATAGTAAGGgctaagcAGCTTGGCGTAACTAG | |
| | ATCT | |
| Phenylketonuria; | ggccaaagcatgcatGAGCGAACTGAGAAGGGCCAGTTTCAGAGCT | 77 |
| not | ATGCTGGAAACAGCATAGCAAGTTGAAATAAGGCTAGTCCGTT | |
| provided | ATCAACTTGAAAAAGTGGCACCGAGTCGGTGCCACAATACCTC | |
| | GGCCCTTCTCAGTTCAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTAGCCTGTGGTTTTGG | |
| | TCTTAGGAACTTTGCTGCCACAATACCTTGGCCCTTCTCAGTT | |
| | CGCTACGACCCATATACAGCgctaagcAGCTTGGCGTAACTAGAT | |
| | CT | |
| Phenylketonuria; | ggccaaagcatgcatGAGCGAACTGAGAAGGGCCAGTTTCAGAGCT | 78 |
| not | ATGCTGGAAACAGCATAGCAAGTTGAAATAAGGCTAGTCCGTT | |
| provided | ATCAACTTGAAAAAGTGGCACCGAGTCGGTGCGCTGCCACAA | |
| | TACCTCGGCCCTTCTCAGTTCAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTGTGGTTTTG | |
| | GTCTTAGGAACTTTGCTGCCACAATACCTTGGCCCTTCTCAGT | |
| | TCGCTACGACCCATACCCCGAgctaagcAGCTTGGCGTAACTAG | |
| | ATCT | |
| Hyperphenyl | ggccaaagcatgcatGCCTCAATCCTTTGGGTGTAGTTTCAGAGCTA | 79 |
| alaninemia, | TGCTGGAAACAGCATAGCAAGTTGAAATAAGGCTAGTCCGTTA | |
| non- | TCAACTTGAAAAAGTGGCACCGAGTCGGTGCGCTACGACCCA | |
| pku; | TACACCCAAAGGATTGAAAAAAAAAGCTGCCATCAGTCGGCGT | |
| Phenylketonuria; | GGACTGTAGAACACTGCCAATGCCGGTCCCAAGCCCGGATAA | |
| not provided | AAGTGGAGGGTACAGTCCACGCTTTTTTTGCTGCCACAATACC | |
| | TCGGCCCTTCTCAGTTCGCTGCGACCCATACACCCAAAGGATT | |
| | GAGGTCTTGGACAACTGCAGgctaagcAGCTTGGCGTAACTAGA | |
| | TCT | |
| Hyperphenyl | ggccaaagcatgcatGCCTCAATCCTTTGGGTGTAGTTTCAGAGCTA | 80 |
| alaninemia, | TGCTGGAAACAGCATAGCAAGTTGAAATAAGGCTAGTCCGTTA | |
| non- | TCAACTTGAAAAAGTGGCACCGAGTCGGTGCGTTCGCTACGA | |
| pku; | CCCATACACCCAAAGGATTGAAAAAAAAAGCTGCCATCAGTCG | |
| Phenylketonuria; | GCGTGGACTGTAGAACACTGCCAATGCCGGTCCCAAGCCCGG | |
| not provided | ATAAAAGTGGAGGGTACAGTCCACGCTTTTTTTCCACAATACC | |
| | TCGGCCCTTCTCAGTTCGCTGCGACCCATACACCCAAAGGATT | |
| | GAGGTCTTGGACAACTGCTGgctaagcAGCTTGGCGTAACTAGA | |
| | TCT | |
| Hyperphenyl | ggccaaagcatgcatGCCTCAATCCTTTGGGTGTAGTTTCAGAGCTA | 81 |
| alaninemia, | TGCTGGAAACAGCATAGCAAGTTGAAATAAGGCTAGTCCGTTA | |
| non- | TCAACTTGAAAAAGTGGCACCGAGTCGGTGCCCTTCTCAGTTC | |
| pku; | GCTACGACCCATACACCCAAAGGATTGAAAAAAAAAGCTGCCA | |
| Phenylketonuria; | TCAGTCGGCGTGGACTGTAGAACACTGCCAATGCCGGTCCCA | |
| not provided | AGCCCGGATAAAAGTGGAGGGTACAGTCCACGCTTTTTTTCCT | |
| | CGGCCCTTCTCAGTTCGCTGCGACCCATACACCCAAAGGATT | |
| | GAGGTCTTGGACAACAGATGgctaagcAGCTTGGCGTAACTAGA | |
| | TCT | |
| Hyperphenyl | ggccaaagcatgcatGCTCAATCCTTTGGGTGTATGTTTCAGAGCTA | 82 |
| alaninemia, | TGCTGGAAACAGCATAGCAAGTTGAAATAAGGCTAGTCCGTTA | |
| non- | TCAACTTGAAAAAGTGGCACCGAGTCGGTGCGCTACGACCCA | |
| pku; | TACACCCAAAGGATTAAAAAAAAAGCTGCCATCAGTCGGCGTG | |
| Phenylketonuria; | GACTGTAGAACACTGCCAATGCCGGTCCCAAGCCCGGATAAA | |
| not provided | AGTGGAGGGTACAGTCCACGCTTTTTTTTTGCTGCCACAATAC | |
| | CTCGGCCCTTCTCAGTTCGCTGCGACCCATACACCCAAAGGA | |
| | TTGAGGTCTTGGACAGCACGCgctaagcAGCTTGGCGTAACTAG | |
| | ATCT | |
| Hyperphenyl | ggccaaagcatgcatGCTCAATCCTTTGGGTGTATGTTTCAGAGCTA | 83 |
| alaninemia, | TGCTGGAAACAGCATAGCAAGTTGAAATAAGGCTAGTCCGTTA | |
| non- | TCAACTTGAAAAAGTGGCACCGAGTCGGTGCGTTCGCTACGA | |
| pku; | CCCATACACCCAAAGGATTAAAAAAAAAGCTGCCATCAGTCGG | |
| Phenylketonuria; | CGTGGACTGTAGAACACTGCCAATGCCGGTCCCAAGCCCGGA | |
| not provided | TAAAAGTGGAGGGTACAGTCCACGCTTTTTTTTGCCACAATAC | |
| | CTCGGCCCTTCTCAGTTCGCTGCGACCCATACACCCAAAGGA | |
| | TTGAGGTCTTGGACAATCGGGgctaagcAGCTTGGCGTAACTAG | |
| | ATCT | |
| Hyperphenyl | ggccaaagcatgcatGCTCAATCCTTTGGGTGTATGTTTCAGAGCTA | 84 |
| alaninemia, | TGCTGGAAACAGCATAGCAAGTTGAAATAAGGCTAGTCCGTTA | |
| non- | TCAACTTGAAAAAGTGGCACCGAGTCGGTGCTCAGTTCGCTAC | |
| pku; | GACCCATACACCCAAAGGATTAAAAAAAAAGCTGCCATCAGTC | |
| Phenylketonuria; | GGCGTGGACTGTAGAACACTGCCAATGCCGGTCCCAAGCCCG | |
| not provided | GATAAAAGTGGAGGGTACAGTCCACGCTTTTTTTCACAATACC | |
| | TCGGCCCTTCTCAGTTCGCTGCGACCCATACACCCAAAGGATT | |
| | GAGGTCTTGGACACGAGTTgctaagcAGCTTGGCGTAACTAGAT | |
| | CT | |
| Hyperphenyl | ggccaaagcatgcatGCTCAATCCTTTGGGTGTATGTTTCAGAGCTA | 85 |
| alaninemia, | TGCTGGAAACAGCATAGCAAGTTGAAATAAGGCTAGTCCGTTA | |
| non- | TCAACTTGAAAAAGTGGCACCGAGTCGGTGCCCTTCTCAGTTC | |
| pku; | GCTACGACCCATACACCCAAAGGATTAAAAAAAAAGCTGCCAT | |
| Phenylketonuria; | CAGTCGGCGTGGACTGTAGAACACTGCCAATGCCGGTCCCAA | |
| not provided | GCCCGGATAAAAGTGGAGGGTACAGTCCACGCTTTTTTTTACC | |
| | TCGGCCCTTCTCAGTTCGCTGCGACCCATACACCCAAAGGATT | |
| | GAGGTCTTGGACAGGGGGAgctaagcAGCTTGGCGTAACTAGAT | |
| | CT | |
| Hyperphenyl | ggccaaagcatgcatGTGCCACGTAATAGAGGGGCGTTTCAGAGCT | 86 |
| alaninemia, | ATGCTGGAAACAGCATAGCAAGTTGAAATAAGGCTAGTCCGTT | |
| non- | ATCAACTTGAAAAAGTGGCACCGAGTCGGTGCTCACGGAGTT | |
| pku; | CCAGCCCCTCTATTACGTGAAAAAAAAAGCTGCCATCAGTCGG | |
| Marfanoid habitus | CGTGGACTGTAGAACACTGCCAATGCCGGTCCCAAGCCCGGA | |
| and | TAAAAGTGGAGGGTACAGTCCACGCTTTTTTTAGAAGACAGCC | |
| intellectual | ATCCAAAATTACACTGTCATGGAGTTCCAGCCCCTCTATTACG | |
| disability; | TGGCAGAGAGTTTTACGTTCTgctaagcAGCTTGGCGTAACTAGA | |
| Phenylketonuria; | TCT | |
| not | | |
| provided | | |
| Hyperphenyl | ggccaaagcatgcatGTGCCACGTAATAGAGGGGCGTTTCAGAGCT | 87 |
| alaninemia, | ATGCTGGAAACAGCATAGCAAGTTGAAATAAGGCTAGTCCGTT | |
| non- | ATCAACTTGAAAAAGTGGCACCGAGTCGGTGCTACACTGTCAC | |
| pku; | GGAGTTCCAGCCCCTCTATTACGTGAAAAAAAAAGCTGCCATC | |
| Marfanoid habitus | AGTCGGCGTGGACTGTAGAACACTGCCAATGCCGGTCCCAAG | |
| and | CCCGGATAAAAGTGGAGGGTACAGTCCACGCTTTTTTTAGCCA | |
| intellectual | TCCAAAATTACACTGTCATGGAGTTCCAGCCCCTCTATTACGT | |
| disability; | GGCAGAGAGTTTTAAAGTTGgctaagcAGCTTGGCGTAACTAGAT | |
| Phenylketonuria; | CT | |
| not | | |
| provided | | |
| Hyperphenyl | ggccaaagcatgcatGTGCCACGTAATAGAGGGGCGTTTCAGAGCT | 88 |
| alaninemia, | ATGCTGGAAACAGCATAGCAAGTTGAAATAAGGCTAGTCCGTT | |
| non- | ATCAACTTGAAAAAGTGGCACCGAGTCGGTGCAAAATTACACT | |
| pku; | GTCACGGAGTTCCAGCCCCTCTATTACGTGAAAAAAAAAGCTG | |
| Marfanoid habitus | CCATCAGTCGGCGTGGACTGTAGAACACTGCCAATGCCGGTC | |
| and | CCAAGCCCGGATAAAAGTGGAGGGTACAGTCCACGCTTTTTTT | |
| intellectual | TCCAAAATTACACTGTCATGGAGTTCCAGCCCCTCTATTACGT | |
| disability; | GGCAGAGAGTTTTAGATTGTgctaagcAGCTTGGCGTAACTAGAT | |
| Phenylketonuria; | CT | |
| not | | |
| provided | | |
| Hyperphenyl | ggccaaagcatgcatGATCCAAAATTACACTGTCAGTTTCAGAGCTAT | 89 |
| alaninemia, | GCTGGAAACAGCATAGCAAGTTGAAATAAGGCTAGTCCGTTAT | |
| non- | CAACTTGAAAAAGTGGCACCGAGTCGGTGCTCCGTGACAGTG | |
| pku; | TAATTTTGAAAAAAAAAGCTGCCATCAGTCGGCGTGGACTGTA | |
| Marfanoid habitus | GAACACTGCCAATGCCGGTCCCAAGCCCGGATAAAAGTGGAG | |
| and | GGTACAGTCCACGCTTTTTTTTTGGCATCATTAAAACTCTCTGC | |
| intellectual | CACGTAATAGAGGGGCTGGAACTCCATGACAGTGTAATTTTGG | |
| disability; | ATGGCTGTCTTCAACACCgctaagcAGCTTGGCGTAACTAGATCT | |
| Phenylketonuria; | | |
| not | | |
| provided | | |
| Hyperphenyl | ggccaaagcatgcatGATCCAAAATTACACTGTCAGTTTCAGAGCTAT | 90 |
| alaninemia, | GCTGGAAACAGCATAGCAAGTTGAAATAAGGCTAGTCCGTTAT | |
| non- | CAACTTGAAAAAGTGGCACCGAGTCGGTGCGAACTCCGTGAC | |
| pku; | AGTGTAATTTTGAAAAAAAAAGCTGCCATCAGTCGGCGTGGAC | |
| Marfanoid habitus | TGTAGAACACTGCCAATGCCGGTCCCAAGCCCGGATAAAAGT | |
| and | GGAGGGTACAGTCCACGCTTTTTTTCATCATTAAAACTCTCTG | |
| intellectual | CCACGTAATAGAGGGGCTGGAACTCCATGACAGTGTAATTTTG | |
| disability; | GATGGCTGTCTTCTATAATgctaagcAGCTTGGCGTAACTAGATC | |
| Phenylketonuria; | T | |
| not | | |
| provided | | |
| Hyperphenyl | ggccaaagcatgcatGATCCAAAATTACACTGTCAGTTTCAGAGCTAT | 91 |
| alaninemia, | GCTGGAAACAGCATAGCAAGTTGAAATAAGGCTAGTCCGTTAT | |
| non- | CAACTTGAAAAAGTGGCACCGAGTCGGTGCCTGGAACTCCGT | |
| pku; | GACAGTGTAATTTTGAAAAAAAAAGCTGCCATCAGTCGGCGTG | |
| Marfanoid habitus | GACTGTAGAACACTGCCAATGCCGGTCCCAAGCCCGGATAAA | |
| and | AGTGGAGGGTACAGTCCACGCTTTTTTTCATTAAAACTCTCTG | |
| intellectual | CCACGTAATAGAGGGGCTGGAACTCCATGACAGTGTAATTTTG | |
| disability; | GATGGCTGTCTTCCAGTCAgctaagcAGCTTGGCGTAACTAGATC | |
| Phenylketonuria; | T | |
| not | | |
| provided | | |
| Hyperphenyl | ggccaaagcatgcatGATCCAAAATTACACTGTCAGTTTCAGAGCTAT | 92 |
| alaninemia, | GCTGGAAACAGCATAGCAAGTTGAAATAAGGCTAGTCCGTTAT | |
| non- | CAACTTGAAAAAGTGGCACCGAGTCGGTGCAGGGGCTGGAAC | |
| pku; | TCCGTGACAGTGTAATTTTGAAAAAAAAAGCTGCCATCAGTCG | |
| Marfanoid habitus | GCGTGGACTGTAGAACACTGCCAATGCCGGTCCCAAGCCCGG | |
| and | ATAAAAGTGGAGGGTACAGTCCACGCTTTTTTTAAACTCTCTG | |
| intellectual | CCACGTAATAGAGGGGCTGGAACTCCATGACAGTGTAATTTTG | |
| disability; | GATGGCTGTCTTCCACATAgctaagcAGCTTGGCGTAACTAGATC | |
| Phenylketonuria; | T | |
| not | | |
| provided | | |
| Inborn | ggccaaagcatgcatGGAGAAGGGCCGAGGTATTGGTTTCAGAGCT | 93 |
| genetic | ATGCTGGAAACAGCATAGCAAGTTGAAATAAGGCTAGTCCGTT | |
| diseases; | ATCAACTTGAAAAAGTGGCACCGAGTCGGTGCTTGCTGCCAC | |
| Phenylketonuria; | AATACCTCGGCCCTTAAAAAAAAAGCTGCCATCAGTCGGCGTG | |
| not | GACTGTAGAACACTGCCAATGCCGGTCCCAAGCCCGGATAAA | |
| provided | AGTGGAGGGTACAGTCCACGCTTTTTTTCTTCACTCAAGCCTG | |
| | TGGTTTTGGTCTTAGGAACTTTGTTGCCACAATACCTCGGCCC | |
| | TTCTCAGTTCGCTACAGATCAgctaagcAGCTTGGCGTAACTAGA | |
| | TCT | |
| Inborn | ggccaaagcatgcatGGAGAAGGGCCGAGGTATTGGTTTCAGAGCT | 94 |
| genetic | ATGCTGGAAACAGCATAGCAAGTTGAAATAAGGCTAGTCCGTT | |
| diseases; | ATCAACTTGAAAAAGTGGCACCGAGTCGGTGCAACTTTGCTGC | |
| Phenylketonuria; | CACAATACCTCGGCCCTTAAAAAAAAAGCTGCCATCAGTCGGC | |
| not | GTGGACTGTAGAACACTGCCAATGCCGGTCCCAAGCCCGGAT | |
| provided | AAAAGTGGAGGGTACAGTCCACGCTTTTTTTACTCAAGCCTGT | |
| | GGTTTTGGTCTTAGGAACTTTGTTGCCACAATACCTCGGCCCT | |
| | TCTCAGTTCGCTACCGAGATgctaagcAGCTTGGCGTAACTAGAT | |
| | CT | |
| Inborn | ggccaaagcatgcatGGAGAAGGGCCGAGGTATTGGTTTCAGAGCT | 95 |
| genetic | ATGCTGGAAACAGCATAGCAAGTTGAAATAAGGCTAGTCCGTT | |
| diseases; | ATCAACTTGAAAAAGTGGCACCGAGTCGGTGCAGGAACTTTG | |
| Phenylketonuria; | CTGCCACAATACCTCGGCCCTTAAAAAAAAAGCTGCCATCAGT | |
| not | CGGCGTGGACTGTAGAACACTGCCAATGCCGGTCCCAAGCCC | |
| provided | GGATAAAAGTGGAGGGTACAGTCCACGCTTTTTTTCAAGCCTG | |
| | TGGTTTTGGTCTTAGGAACTTTGTTGCCACAATACCTCGGCCC | |
| | TTCTCAGTTCGCTACGGTCGTgctaagcAGCTTGGCGTAACTAGA | |
| | TCT | |
| Inborn | ggccaaagcatgcatGAGCCTGTGGTTTTGGTCTTGTTTCAGAGCTA | 96 |
| genetic | TGCTGGAAACAGCATAGCAAGTTGAAATAAGGCTAGTCCGTTA | |
| diseases; | TCAACTTGAAAAAGTGGCACCGAGTCGGTGCTGTGGCAGCAA | |
| Phenylketonuria; | AGTTCCTAAGACCAAAACCACAGAAAAAAAAAGCTGCCATCAG | |
| not | TCGGCGTGGACTGTAGAACACTGCCAATGCCGGTCCCAAGCC | |
| provided | CGGATAAAAGTGGAGGGTACAGTCCACGCTTTTTTTGAGAAGG | |
| | GCCGAGGTATTGTGGCAACAAAGTTCCTAAGACCAAAACCACA | |
| | GGCTTGAGTGAAGGGCTGGCgctaagcAGCTTGGCGTAACTAGA | |
| | TCT | |
| Inborn | ggccaaagcatgcatGAGCCTGTGGTTTTGGTCTTGTTTCAGAGCTA | 97 |
| genetic | TGCTGGAAACAGCATAGCAAGTTGAAATAAGGCTAGTCCGTTA | |
| diseases; | TCAACTTGAAAAAGTGGCACCGAGTCGGTGCTATTGTGGCAG | |
| Phenylketonuria; | CAAAGTTCCTAAGACCAAAACCACAGAAAAAAAAAGCTGCCAT | |
| not | CAGTCGGCGTGGACTGTAGAACACTGCCAATGCCGGTCCCAA | |
| provided | GCCCGGATAAAAGTGGAGGGTACAGTCCACGCTTTTTTTAAGG | |
| | GCCGAGGTATTGTGGCAACAAAGTTCCTAAGACCAAAACCACA | |
| | GGCTTGAGTGAAGGAGGCTTgctaagcAGCTTGGCGTAACTAGA | |
| | TCT | |
| Inborn | ggccaaagcatgcatGAGCCTGTGGTTTTGGTCTTGTTTCAGAGCTA | 98 |
| genetic | TGCTGGAAACAGCATAGCAAGTTGAAATAAGGCTAGTCCGTTA | |
| diseases; | TCAACTTGAAAAAGTGGCACCGAGTCGGTGCCGAGGTATTGT | |
| Phenylketonuria; | GGCAGCAAAGTTCCTAAGACCAAAACCACAGAAAAAAAAAGCT | |
| not | GCCATCAGTCGGCGTGGACTGTAGAACACTGCCAATGCCGGT | |
| provided | CCCAAGCCCGGATAAAAGTGGAGGGTACAGTCCACGCTTTTTT | |
| | TCCGAGGTATTGTGGCAACAAAGTTCCTAAGACCAAAACCACA | |
| | GGCTTGAGTGAAGGGAATTAgctaagcAGCTTGGCGTAACTAGAT | |
| | CT | |
| Phenylketonuria; | ggccaaagcatgcatGAGAAGGTCTAGATTCAGTGGTTTCAGAGCTA | 99 |
| not | TGCTGGAAACAGCATAGCAAGTTGAAATAAGGCTAGTCCGTTA | |
| provided | TCAACTTGAAAAAGTGGCACCGAGTCGGTGCACATTGAATCTA | |
| | GACCTAAAAAAAAAGCTGCCATCAGTCGGCGTGGACTGTAGA | |
| | ACACTGCCAATGCCGGTCCCAAGCCCGGATAAAAGTGGAGGG | |
| | TACAGTCCACGCTTTTTTTCTCCTCACCCTCCCCATTCTCTCTT | |
| | CTAGGAGAATGATGTAAACCTGACCCACACTGAATCTAGACCT | |
| | TCTCGTTTAAAGACAAGGAgctaagcAGCTTGGCGTAACTAGATC | |
| | T | |
| Phenylketonuria; | ggccaaagcatgcatGAGAAGGTCTAGATTCAGTGGTTTCAGAGCTA | 100 |
| not | TGCTGGAAACAGCATAGCAAGTTGAAATAAGGCTAGTCCGTTA | |
| provided | TCAACTTGAAAAAGTGGCACCGAGTCGGTGCACCCACATTGAA | |
| | TCTAGACCTAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTTCACCCTCCCCATTCTCTCTTC | |
| | TAGGAGAATGATGTAAACCTGACCCACACTGAATCTAGACCTT | |
| | CTCGTTTAAAGACTGCTAgctaagcAGCTTGGCGTAACTAGATCT | |
| Phenylketonuria; | ggccaaagcatgcatGAGAAGGTCTAGATTCAGTGGTTTCAGAGCTA | 101 |
| not | TGCTGGAAACAGCATAGCAAGTTGAAATAAGGCTAGTCCGTTA | |
| provided | TCAACTTGAAAAAGTGGCACCGAGTCGGTGCCTGACCCACATT | |
| | GAATCTAGACCTAAAAAAAAAGCTGCCATCAGTCGGCGTGGAC | |
| | TGTAGAACACTGCCAATGCCGGTCCCAAGCCCGGATAAAAGT | |
| | GGAGGGTACAGTCCACGCTTTTTTTCCCTCCCCATTCTCTCTT | |
| | CTAGGAGAATGATGTAAACCTGACCCACACTGAATCTAGACCT | |
| | TCTCGTTTAAAGAGCTCACgctaagcAGCTTGGCGTAACTAGATC | |
| | T | |
| Phenylketonuria; | ggccaaagcatgcatGAGAAGGTCTAGATTCAGTGGTTTCAGAGCTA | 102 |
| not | TGCTGGAAACAGCATAGCAAGTTGAAATAAGGCTAGTCCGTTA | |
| provided | TCAACTTGAAAAAGTGGCACCGAGTCGGTGCTAAACCTGACC | |
| | CACATTGAATCTAGACCTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTCCCATTCTCTCTT | |
| | CTAGGAGAATGATGTAAACCTGACCCACACTGAATCTAGACCT | |
| | TCTCGTTTAAAGAGAAACTgctaagcAGCTTGGCGTAACTAGATC | |
| | T | |
| Phenylketonuria; | ggccaaagcatgcatGTCTGATGTACTGTGTGCAGGTTTCAGAGCTA | 103 |
| not | TGCTGGAAACAGCATAGCAAGTTGAAATAAGGCTAGTCCGTTA | |
| provided | TCAACTTGAAAAAGTGGCACCGAGTCGGTGCTCCGAGTCTTC | |
| | CACTGCACACAGTACATCAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTCTCGGGATTTCT | |
| | TGGGTGGCCTGGCCTTCCAAGTCTTCCACTGCACACAGTACAT | |
| | CAGACATGGATCCAAAGTGAgctaagcAGCTTGGCGTAACTAGAT | |
| | CT | |
| Phenylketonuria; | ggccaaagcatgcatGTCTGATGTACTGTGTGCAGGTTTCAGAGCTA | 104 |
| not | TGCTGGAAACAGCATAGCAAGTTGAAATAAGGCTAGTCCGTTA | |
| provided | TCAACTTGAAAAAGTGGCACCGAGTCGGTGCGCCTTCCGAGT | |
| | CTTCCACTGCACACAGTACATCAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTGGATTTCT | |
| | TGGGTGGCCTGGCCTTCCAAGTCTTCCACTGCACACAGTACAT | |
| | CAGACATGGATCCACTTCTCgctaagcAGCTTGGCGTAACTAGAT | |
| | CT | |
| Phenylketonuria; | ggccaaagcatgcatGTCTGATGTACTGTGTGCAGGTTTCAGAGCTA | 105 |
| not | TGCTGGAAACAGCATAGCAAGTTGAAATAAGGCTAGTCCGTTA | |
| provided | TCAACTTGAAAAAGTGGCACCGAGTCGGTGCCTGGCCTTCCG | |
| | AGTCTTCCACTGCACACAGTACATCAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTTTTCT | |
| | TGGGTGGCCTGGCCTTCCAAGTCTTCCACTGCACACAGTACAT | |
| | CAGACATGGATCCAACCGGTgctaagcAGCTTGGCGTAACTAGA | |
| | TCT | |
| Phenylketonuria; | ggccaaagcatgcatGTCTGATGTACTGTGTGCAGGTTTCAGAGCTA | 106 |
| not | TGCTGGAAACAGCATAGCAAGTTGAAATAAGGCTAGTCCGTTA | |
| provided | TCAACTTGAAAAAGTGGCACCGAGTCGGTGCGTGGCCTGGCC | |
| | TTCCGAGTCTTCCACTGCACACAGTACATCAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | TGGGTGGCCTGGCCTTCCAAGTCTTCCACTGCACACAGTACAT | |
| | CAGACATGGATCCATGATCGgctaagcAGCTTGGCGTAACTAGAT | |
| | CT | |
| Phenylketonuria; | ggccaaagcatgcatGCTGTGTGCAGTGGAAGACTGTTTCAGAGCTA | 107 |
| not | TGCTGGAAACAGCATAGCAAGTTGAAATAAGGCTAGTCCGTTA | |
| provided | TCAACTTGAAAAAGTGGCACCGAGTCGGTGCTCCGAGTCTTC | |
| | CACTGCACAAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTTGGCTGGCCTGCTTTCCTCT | |
| | CGGGATTTCTTGGGTGGCCTGGCCTTCCAAGTCTTCCACTGC | |
| | ACACAGTACATCAGACGGTCGGgctaagcAGCTTGGCGTAACTA | |
| | GATCT | |
| Phenylketonuria; | ggccaaagcatgcatGCTGTGTGCAGTGGAAGACTGTTTCAGAGCTA | 108 |
| not | TGCTGGAAACAGCATAGCAAGTTGAAATAAGGCTAGTCCGTTA | |
| provided | TCAACTTGAAAAAGTGGCACCGAGTCGGTGCGCCTTCCGAGT | |
| | CTTCCACTGCACAAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTGGCCTGCTTTCCTCTC | |
| | GGGATTTCTTGGGTGGCCTGGCCTTCCAAGTCTTCCACTGCA | |
| | CACAGTACATCAGACTGGACCgctaagcAGCTTGGCGTAACTAG | |
| | ATCT | |
| Phenylketonuria; | ggccaaagcatgcatGCTGTGTGCAGTGGAAGACTGTTTCAGAGCTA | 109 |
| not | TGCTGGAAACAGCATAGCAAGTTGAAATAAGGCTAGTCCGTTA | |
| provided | TCAACTTGAAAAAGTGGCACCGAGTCGGTGCCTGGCCTTCCG | |
| | AGTCTTCCACTGCACAAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTCCTGCTTTCCTCTC | |
| | GGGATTTCTTGGGTGGCCTGGCCTTCCAAGTCTTCCACTGCA | |
| | CACAGTACATCAGACTAGCTAgctaagcAGCTTGGCGTAACTAGA | |
| | TCT | |
| Phenylketonuria; | ggccaaagcatgcatGCTGTGTGCAGTGGAAGACTGTTTCAGAGCTA | 110 |
| not | TGCTGGAAACAGCATAGCAAGTTGAAATAAGGCTAGTCCGTTA | |
| provided | TCAACTTGAAAAAGTGGCACCGAGTCGGTGCGTGGCCTGGCC | |
| | TTCCGAGTCTTCCACTGCACAAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTTTTCCTCTC | |
| | GGGATTTCTTGGGTGGCCTGGCCTTCCAAGTCTTCCACTGCA | |
| | CACAGTACATCAGACCATAATgctaagcAGCTTGGCGTAACTAGA | |
| | TCT | |
| Phenylketonuria; | ggccaaagcatgcatGCGGGATTTCTTGGGTGGCCGTTTCAGAGCT | 111 |
| not | ATGCTGGAAACAGCATAGCAAGTTGAAATAAGGCTAGTCCGTT | |
| provided | ATCAACTTGAAAAAGTGGCACCGAGTCGGTGCACTCGGAAGG | |
| | CCAGGCCACCCAAGAAATCAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTGTCTGATGT | |
| | ACTGTGTGCAGTGGAAGACTTGGAAGGCCAGGCCACCCAAGA | |
| | AATCCCGAGAGGAAAGCAGAATAgctaagcAGCTTGGCGTAACT | |
| | AGATCT | |
| Phenylketonuria; | ggccaaagcatgcatGCGGGATTTCTTGGGTGGCCGTTTCAGAGCT | 112 |
| not | ATGCTGGAAACAGCATAGCAAGTTGAAATAAGGCTAGTCCGTT | |
| provided | ATCAACTTGAAAAAGTGGCACCGAGTCGGTGCGAAGACTCGG | |
| | AAGGCCAGGCCACCCAAGAAATCAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTTGATGT | |
| | ACTGTGTGCAGTGGAAGACTTGGAAGGCCAGGCCACCCAAGA | |
| | AATCCCGAGAGGAAAGCGAACCGgctaagcAGCTTGGCGTAACT | |
| | AGATCT | |
| Phenylketonuria; | ggccaaagcatgcatGCGGGATTTCTTGGGTGGCCGTTTCAGAGCT | 113 |
| not | ATGCTGGAAACAGCATAGCAAGTTGAAATAAGGCTAGTCCGTT | |
| provided | ATCAACTTGAAAAAGTGGCACCGAGTCGGTGCGTGGAAGACT | |
| | CGGAAGGCCAGGCCACCCAAGAAATCAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTTGT | |
| | ACTGTGTGCAGTGGAAGACTTGGAAGGCCAGGCCACCCAAGA | |
| | AATCCCGAGAGGAAAGCGCGAACgctaagcAGCTTGGCGTAACT | |
| | AGATCT | |
| Phenylketonuria; | ggccaaagcatgcatGCGGGATTTCTTGGGTGGCCGTTTCAGAGCT | 114 |
| not | ATGCTGGAAACAGCATAGCAAGTTGAAATAAGGCTAGTCCGTT | |
| provided | ATCAACTTGAAAAAGTGGCACCGAGTCGGTGCGTGCAGTGGA | |
| | AGACTCGGAAGGCCAGGCCACCCAAGAAATCAAAAAAAAAGC | |
| | TGCCATCAGTCGGCGTGGACTGTAGAACACTGCCAATGCCGG | |
| | TCCCAAGCCCGGATAAAAGTGGAGGGTACAGTCCACGCTTTTT | |
| | TTTGTGTGCAGTGGAAGACTTGGAAGGCCAGGCCACCCAAGA | |
| | AATCCCGAGAGGAAAGCGATACCgctaagcAGCTTGGCGTAACT | |
| | AGATCT | |
| Hyperphenyl | ggccaaagcatgcatGATTCCTTACCTGGGAAAACGTTTCAGAGCTA | 115 |
| alaninemia, | TGCTGGAAACAGCATAGCAAGTTGAAATAAGGCTAGTCCGTTA | |
| non- | TCAACTTGAAAAAGTGGCACCGAGTCGGTGCTTTGCCCAGTTT | |
| pku; | TCCCAGGTAAGGAAAAAAAAAGCTGCCATCAGTCGGCGTGGA | |
| Phenylketonuria; | CTGTAGAACACTGCCAATGCCGGTCCCAAGCCCGGATAAAAG | |
| not provided | TGGAGGGTACAGTCCACGCTTTTTTTGCTGTTGGGACATGTGC | |
| | CCTTGTTTTCAGATCGCAGCTTTTCCCAGTTTTCCCAGGTAAG | |
| | GAATGGATTTTTTATCAAGCgctaagcAGCTTGGCGTAACTAGAT | |
| | CT | |
| Hyperphenyl | ggccaaagcatgcatGATTCCTTACCTGGGAAAACGTTTCAGAGCTA | 116 |
| alaninemia, | TGCTGGAAACAGCATAGCAAGTTGAAATAAGGCTAGTCCGTTA | |
| non- | TCAACTTGAAAAAGTGGCACCGAGTCGGTGCCAGCTTTGCCC | |
| pku; | AGTTTTCCCAGGTAAGGAAAAAAAAAGCTGCCATCAGTCGGCG | |
| Phenylketonuria; | TGGACTGTAGAACACTGCCAATGCCGGTCCCAAGCCCGGATA | |
| not provided | AAAGTGGAGGGTACAGTCCACGCTTTTTTTTTGGGACATGTGC | |
| | CCTTGTTTTCAGATCGCAGCTTTTCCCAGTTTTCCCAGGTAAG | |
| | GAATGGATTTTTTAGACTACgctaagcAGCTTGGCGTAACTAGAT | |
| | CT | |
| Hyperphenyl | ggccaaagcatgcatGATTCCTTACCTGGGAAAACGTTTCAGAGCTA | 117 |
| alaninemia, | TGCTGGAAACAGCATAGCAAGTTGAAATAAGGCTAGTCCGTTA | |
| non- | TCAACTTGAAAAAGTGGCACCGAGTCGGTGCTCGCAGCTTTG | |
| pku; | CCCAGTTTTCCCAGGTAAGGAAAAAAAAAGCTGCCATCAGTCG | |
| Phenylketonuria; | GCGTGGACTGTAGAACACTGCCAATGCCGGTCCCAAGCCCGG | |
| not provided | ATAAAAGTGGAGGGTACAGTCCACGCTTTTTTTGGACATGTGC | |
| | CCTTGTTTTCAGATCGCAGCTTTTCCCAGTTTTCCCAGGTAAG | |
| | GAATGGATTTTTTACATAGAgctaagcAGCTTGGCGTAACTAGAT | |
| | CT | |
| Hyperphenyl | ggccaaagcatgcatGATTCCTTACCTGGGAAAACGTTTCAGAGCTA | 118 |
| alaninemia, | TGCTGGAAACAGCATAGCAAGTTGAAATAAGGCTAGTCCGTTA | |
| non- | TCAACTTGAAAAAGTGGCACCGAGTCGGTGCTCAGATCGCAG | |
| pku; | CTTTGCCCAGTTTTCCCAGGTAAGGAAAAAAAAAGCTGCCATC | |
| Phenylketonuria; | AGTCGGCGTGGACTGTAGAACACTGCCAATGCCGGTCCCAAG | |
| not provided | CCCGGATAAAAGTGGAGGGTACAGTCCACGCTTTTTTTTGTGC | |
| | CCTTGTTTTCAGATCGCAGCTTTTCCCAGTTTTCCCAGGTAAG | |
| | GAATGGATTTTTTAGATTCCgctaagcAGCTTGGCGTAACTAGAT | |
| | CT | |
| Hyperphenyl | ggccaaagcatgcatGTTCCTTACCTGGGAAAACTGTTTCAGAGCTA | 119 |
| alaninemia, | TGCTGGAAACAGCATAGCAAGTTGAAATAAGGCTAGTCCGTTA | |
| non- | TCAACTTGAAAAAGTGGCACCGAGTCGGTGCTTTGCCCAGTTT | |
| pku; | TCCCAGGTAAGAAAAAAAAAGCTGCCATCAGTCGGCGTGGAC | |
| Phenylketonuria; | TGTAGAACACTGCCAATGCCGGTCCCAAGCCCGGATAAAAGT | |
| not provided | GGAGGGTACAGTCCACGCTTTTTTTGAGCTGTTGGGACATGTG | |
| | CCCTTGTTTTCAGATCGCAGCTTTTCCCAGTTTTCCCAGGTAA | |
| | GGAATGGATTTTTTTGCCTTgctaagcAGCTTGGCGTAACTAGAT | |
| | CT | |
| Hyperphenyl | ggccaaagcatgcatGTTCCTTACCTGGGAAAACTGTTTCAGAGCTA | 120 |
| alaninemia, | TGCTGGAAACAGCATAGCAAGTTGAAATAAGGCTAGTCCGTTA | |
| non- | TCAACTTGAAAAAGTGGCACCGAGTCGGTGCCAGCTTTGCCC | |
| pku; | AGTTTTCCCAGGTAAGAAAAAAAAAGCTGCCATCAGTCGGCGT | |
| Phenylketonuria; | GGACTGTAGAACACTGCCAATGCCGGTCCCAAGCCCGGATAA | |
| not provided | AAGTGGAGGGTACAGTCCACGCTTTTTTTTGTTGGGACATGTG | |
| | CCCTTGTTTTCAGATCGCAGCTTTTCCCAGTTTTCCCAGGTAA | |
| | GGAATGGATTTTTTTGTCTCgctaagcAGCTTGGCGTAACTAGAT | |
| | CT | |
| Hyperphenyl | ggccaaagcatgcatGTTCCTTACCTGGGAAAACTGTTTCAGAGCTA | 121 |
| alaninemia, | TGCTGGAAACAGCATAGCAAGTTGAAATAAGGCTAGTCCGTTA | |
| non- | TCAACTTGAAAAAGTGGCACCGAGTCGGTGCTCGCAGCTTTG | |
| pku; | CCCAGTTTTCCCAGGTAAGAAAAAAAAAGCTGCCATCAGTCGG | |
| Phenylketonuria; | CGTGGACTGTAGAACACTGCCAATGCCGGTCCCAAGCCCGGA | |
| not provided | TAAAAGTGGAGGGTACAGTCCACGCTTTTTTTTGGGACATGTG | |
| | CCCTTGTTTTCAGATCGCAGCTTTTCCCAGTTTTCCCAGGTAA | |
| | GGAATGGATTTTTTGGAGGTgctaagcAGCTTGGCGTAACTAGAT | |
| | CT | |
| Hyperphenyl | ggccaaagcatgcatGCCTCAATCCTTTGGGTGTAGTTTCAGAGCTA | 122 |
| alaninemia, | TGCTGGAAACAGCATAGCAAGTTGAAATAAGGCTAGTCCGTTA | |
| non- | TCAACTTGAAAAAGTGGCACCGAGTCGGTGCTACGACCCATA | |
| pku; | CACCCAAAGGATTGAAAAAAAAAGCTGCCATCAGTCGGCGTG | |
| Phenylketonuria; | GACTGTAGAACACTGCCAATGCCGGTCCCAAGCCCGGATAAA | |
| not provided | AGTGGAGGGTACAGTCCACGCTTTTTTTTTGCTGCCACAATAC | |
| | CTCGGCCCTTCTCAGTTCGCTACAACCCATACACCCAAAGGAT | |
| | TGAGGTCTTGGACAACCTGCAgctaagcAGCTTGGCGTAACTAG | |
| | ATCT | |
| Hyperphenyl | ggccaaagcatgcatGCCTCAATCCTTTGGGTGTAGTTTCAGAGCTA | 123 |
| alaninemia, | TGCTGGAAACAGCATAGCAAGTTGAAATAAGGCTAGTCCGTTA | |
| non- | TCAACTTGAAAAAGTGGCACCGAGTCGGTGCTCGCTACGACC | |
| pku; | CATACACCCAAAGGATTGAAAAAAAAAGCTGCCATCAGTCGGC | |
| Phenylketonuria; | GTGGACTGTAGAACACTGCCAATGCCGGTCCCAAGCCCGGAT | |
| not provided | AAAAGTGGAGGGTACAGTCCACGCTTTTTTTTGCCACAATACC | |
| | TCGGCCCTTCTCAGTTCGCTACAACCCATACACCCAAAGGATT | |
| | GAGGTCTTGGACAAAACGCCgctaagcAGCTTGGCGTAACTAGA | |
| | TCT | |
| Hyperphenyl | ggccaaagcatgcatGCTCAATCCTTTGGGTGTATGTTTCAGAGCTA | 124 |
| alaninemia, | TGCTGGAAACAGCATAGCAAGTTGAAATAAGGCTAGTCCGTTA | |
| non- | TCAACTTGAAAAAGTGGCACCGAGTCGGTGCTACGACCCATA | |
| pku; | CACCCAAAGGATTAAAAAAAAAGCTGCCATCAGTCGGCGTGG | |
| Phenylketonuria; | ACTGTAGAACACTGCCAATGCCGGTCCCAAGCCCGGATAAAA | |
| not provided | GTGGAGGGTACAGTCCACGCTTTTTTTCTTTGCTGCCACAATA | |
| | CCTCGGCCCTTCTCAGTTCGCTACAACCCATACACCCAAAGGA | |
| | TTGAGGTCTTGGACACGTTTCgctaagcAGCTTGGCGTAACTAGA | |
| | TCT | |
| Hyperphenyl | ggccaaagcatgcatGCTCAATCCTTTGGGTGTATGTTTCAGAGCTA | 125 |
| alaninemia, | TGCTGGAAACAGCATAGCAAGTTGAAATAAGGCTAGTCCGTTA | |
| non- | TCAACTTGAAAAAGTGGCACCGAGTCGGTGCTCGCTACGACC | |
| pku; | CATACACCCAAAGGATTAAAAAAAAAGCTGCCATCAGTCGGCG | |
| Phenylketonuria; | TGGACTGTAGAACACTGCCAATGCCGGTCCCAAGCCCGGATA | |
| not provided | AAAGTGGAGGGTACAGTCCACGCTTTTTTTGCTGCCACAATAC | |
| | CTCGGCCCTTCTCAGTTCGCTACAACCCATACACCCAAAGGAT | |
| | TGAGGTCTTGGACAGTGGCAgctaagcAGCTTGGCGTAACTAGA | |
| | TCT | |
| Hyperphenyl | ggccaaagcatgcatGCTCAATCCTTTGGGTGTATGTTTCAGAGCTA | 126 |
| alaninemia, | TGCTGGAAACAGCATAGCAAGTTGAAATAAGGCTAGTCCGTTA | |
| non- | TCAACTTGAAAAAGTGGCACCGAGTCGGTGCAGTTCGCTACG | |
| pku; | ACCCATACACCCAAAGGATTAAAAAAAAAGCTGCCATCAGTCG | |
| Phenylketonuria; | GCGTGGACTGTAGAACACTGCCAATGCCGGTCCCAAGCCCGG | |
| not provided | ATAAAAGTGGAGGGTACAGTCCACGCTTTTTTTGCCACAATAC | |
| | CTCGGCCCTTCTCAGTTCGCTACAACCCATACACCCAAAGGAT | |
| | TGAGGTCTTGGACACACACTgctaagcAGCTTGGCGTAACTAGAT | |
| | CT | |
| Hyperphenyl | ggccaaagcatgcatGCTCAATCCTTTGGGTGTATGTTTCAGAGCTA | 127 |
| alaninemia, | TGCTGGAAACAGCATAGCAAGTTGAAATAAGGCTAGTCCGTTA | |
| non- | TCAACTTGAAAAAGTGGCACCGAGTCGGTGCTTCTCAGTTCGC | |
| pku; | TACGACCCATACACCCAAAGGATTAAAAAAAAAGCTGCCATCA | |
| Phenylketonuria; | GTCGGCGTGGACTGTAGAACACTGCCAATGCCGGTCCCAAGC | |
| not provided | CCGGATAAAAGTGGAGGGTACAGTCCACGCTTTTTTTAATACC | |
| | TCGGCCCTTCTCAGTTCGCTACAACCCATACACCCAAAGGATT | |
| | GAGGTCTTGGACAAATGCAgctaagcAGCTTGGCGTAACTAGATC | |
| | T | |
| Phenylketonuria; | ggccaaagcatgcatGAGCTGGAGGACAGTACTCAGTTTCAGAGCTA | 128 |
| not | TGCTGGAAACAGCATAGCAAGTTGAAATAAGGCTAGTCCGTTA | |
| provided | TCAACTTGAAAAAGTGGCACCGAGTCGGTGCCCCGAACCGTG | |
| | AGTACTGTCCTCCAAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTAGTACATCAGACATGG | |
| | ATCCAAGCCCATGTATACCCCCAAACCGTGAGTACTGTCCTCC | |
| | AGCTACCAGTTGCCCACTCAgctaagcAGCTTGGCGTAACTAGAT | |
| | CT | |
| Phenylketonuria; | ggccaaagcatgcatGAGCTGGAGGACAGTACTCAGTTTCAGAGCTA | 129 |
| not | TGCTGGAAACAGCATAGCAAGTTGAAATAAGGCTAGTCCGTTA | |
| provided | TCAACTTGAAAAAGTGGCACCGAGTCGGTGCTACCCCCGAAC | |
| | CGTGAGTACTGTCCTCCAAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTCATCAGACATGG | |
| | ATCCAAGCCCATGTATACCCCCAAACCGTGAGTACTGTCCTCC | |
| | AGCTACCAGTTGCCGGTGGGgctaagcAGCTTGGCGTAACTAGA | |
| | TCT | |
| Phenylketonuria; | ggccaaagcatgcatGAGGACAGTACTCACGGTTTGTTTCAGAGCTA | 130 |
| not | TGCTGGAAACAGCATAGCAAGTTGAAATAAGGCTAGTCCGTTA | |
| provided | TCAACTTGAAAAAGTGGCACCGAGTCGGTGCCCCGAACCGTG | |
| | AGTACTGTAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTTCCACTGCACACAGTACATCAG | |
| | ACATGGATCCAAGCCCATGTATACCCCCAAACCGTGAGTACTG | |
| | TCCTCCAGCTACCACTCAAAgctaagcAGCTTGGCGTAACTAGAT | |
| | CT | |
| Phenylketonuria; | ggccaaagcatgcatGAGGACAGTACTCACGGTTTGTTTCAGAGCTA | 131 |
| not | TGCTGGAAACAGCATAGCAAGTTGAAATAAGGCTAGTCCGTTA | |
| provided | TCAACTTGAAAAAGTGGCACCGAGTCGGTGCTACCCCCGAAC | |
| | CGTGAGTACTGTAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTCTGCACACAGTACATCA | |
| | GACATGGATCCAAGCCCATGTATACCCCCAAACCGTGAGTACT | |
| | GTCCTCCAGCTACCATGAAGCgctaagcAGCTTGGCGTAACTAG | |
| | ATCT | |
| Phenylketonuria; | ggccaaagcatgcatGAGGACAGTACTCACGGTTTGTTTCAGAGCTA | 132 |
| not | TGCTGGAAACAGCATAGCAAGTTGAAATAAGGCTAGTCCGTTA | |
| provided | TCAACTTGAAAAAGTGGCACCGAGTCGGTGCGTATACCCCCG | |
| | AACCGTGAGTACTGTAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTCACACAGTACATCAG | |
| | ACATGGATCCAAGCCCATGTATACCCCCAAACCGTGAGTACTG | |
| | TCCTCCAGCTACCAGATGTGgctaagcAGCTTGGCGTAACTAGAT | |
| | CT | |
| Phenylketonuria; | ggccaaagcatgcatGAGGACAGTACTCACGGTTTGTTTCAGAGCTA | 133 |
| not | TGCTGGAAACAGCATAGCAAGTTGAAATAAGGCTAGTCCGTTA | |
| provided | TCAACTTGAAAAAGTGGCACCGAGTCGGTGCCCCATGTATACC | |
| | CCCGAACCGTGAGTACTGTAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTAGTACATCAG | |
| | ACATGGATCCAAGCCCATGTATACCCCCAAACCGTGAGTACTG | |
| | TCCTCCAGCTACCAAGCCATgctaagcAGCTTGGCGTAACTAGAT | |
| | CT | |
| Phenylketonuria; | ggccaaagcatgcatGGGACAGTACTCACGGTTTGGTTTCAGAGCTA | 134 |
| not | TGCTGGAAACAGCATAGCAAGTTGAAATAAGGCTAGTCCGTTA | |
| provided | TCAACTTGAAAAAGTGGCACCGAGTCGGTGCCCCGAACCGTG | |
| | AGTACTGAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTCTTCCACTGCACACAGTACATC | |
| | AGACATGGATCCAAGCCCATGTATACCCCCAAACCGTGAGTAC | |
| | TGTCCTCCAGCTACCCTCTATgctaagcAGCTTGGCGTAACTAGA | |
| | TCT | |
| Phenylketonuria; | ggccaaagcatgcatGGGACAGTACTCACGGTTTGGTTTCAGAGCTA | 135 |
| not | TGCTGGAAACAGCATAGCAAGTTGAAATAAGGCTAGTCCGTTA | |
| provided | TCAACTTGAAAAAGTGGCACCGAGTCGGTGCTACCCCCGAAC | |
| | CGTGAGTACTGAAAAAAAAAGCTGCCATCAGTCGGCGTGGAC | |
| | TGTAGAACACTGCCAATGCCGGTCCCAAGCCCGGATAAAAGT | |
| | GGAGGGTACAGTCCACGCTTTTTTTCACTGCACACAGTACATC | |
| | AGACATGGATCCAAGCCCATGTATACCCCCAAACCGTGAGTAC | |
| | TGTCCTCCAGCTACCGTATGGgctaagcAGCTTGGCGTAACTAGA | |
| | TCT | |
| Phenylketonuria; | ggccaaagcatgcatGGGACAGTACTCACGGTTTGGTTTCAGAGCTA | 136 |
| not | TGCTGGAAACAGCATAGCAAGTTGAAATAAGGCTAGTCCGTTA | |
| provided | TCAACTTGAAAAAGTGGCACCGAGTCGGTGCGTATACCCCCG | |
| | AACCGTGAGTACTGAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTGCACACAGTACATC | |
| | AGACATGGATCCAAGCCCATGTATACCCCCAAACCGTGAGTAC | |
| | TGTCCTCCAGCTACCCCTGTTgctaagcAGCTTGGCGTAACTAGA | |
| | TCT | |
| Phenylketonuria; | ggccaaagcatgcatGGGACAGTACTCACGGTTTGGTTTCAGAGCTA | 137 |
| not | TGCTGGAAACAGCATAGCAAGTTGAAATAAGGCTAGTCCGTTA | |
| provided | TCAACTTGAAAAAGTGGCACCGAGTCGGTGCCCCATGTATACC | |
| | CCCGAACCGTGAGTACTGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTACAGTACATCA | |
| | GACATGGATCCAAGCCCATGTATACCCCCAAACCGTGAGTACT | |
| | GTCCTCCAGCTACCATTTAGgctaagcAGCTTGGCGTAACTAGAT | |
| | CT | |
| Phenylketonuria; | ggccaaagcatgcatGCTCTGATAAGCAGTACTGTGTTTCAGAGCTA | 138 |
| not | TGCTGGAAACAGCATAGCAAGTTGAAATAAGGCTAGTCCGTTA | |
| provided | TCAACTTGAAAAAGTGGCACCGAGTCGGTGCCTTGGGGCCTA | |
| | CAGTACTGCTTATCAAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTGATCCTGATTTAAC | |
| | AGTGATAATAACTTTTCACTTAGGGCCTACAGTACTGCTTATCA | |
| | GAGAAGCCAAAGCCACCAGgctaagcAGCTTGGCGTAACTAGAT | |
| | CT | |
| Phenylketonuria; | ggccaaagcatgcatGCTCTGATAAGCAGTACTGTGTTTCAGAGCTA | 139 |
| not | TGCTGGAAACAGCATAGCAAGTTGAAATAAGGCTAGTCCGTTA | |
| provided | TCAACTTGAAAAAGTGGCACCGAGTCGGTGCTTCACTTGGGG | |
| | CCTACAGTACTGCTTATCAAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTCCTGATTTAACA | |
| | GTGATAATAACTTTTCACTTAGGGCCTACAGTACTGCTTATCAG | |
| | AGAAGCCAAAGCTAAGCAgctaagcAGCTTGGCGTAACTAGATCT | |
| Phenylketonuria; | ggccaaagcatgcatGCTCTGATAAGCAGTACTGTGTTTCAGAGCTA | 140 |
| not | TGCTGGAAACAGCATAGCAAGTTGAAATAAGGCTAGTCCGTTA | |
| provided | TCAACTTGAAAAAGTGGCACCGAGTCGGTGCCTTTTCACTTGG | |
| | GGCCTACAGTACTGCTTATCAAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTGATTTAACA | |
| | GTGATAATAACTTTTCACTTAGGGCCTACAGTACTGCTTATCAG | |
| | AGAAGCCAAAGCCAAGTGgctaagcAGCTTGGCGTAACTAGATC | |
| | T | |
| Phenylketonuria; | ggccaaagcatgcatGTGATAATAACTTTTCACTTGTTTCAGAGCTAT | 141 |
| not | GCTGGAAACAGCATAGCAAGTTGAAATAAGGCTAGTCCGTTAT | |
| provided | CAACTTGAAAAAGTGGCACCGAGTCGGTGCCCCCAAGTGAAA | |
| | AGTTATTAAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTTCCAGGGGGAGAAGCTTTGGCT | |
| | TCTCTGATAAGCAGTACTGTAGGCCCTAAGTGAAAAGTTATTAT | |
| | CACTGTTAAATCATCGACgctaagcAGCTTGGCGTAACTAGATCT | |
| Phenylketonuria; | ggccaaagcatgcatGTGATAATAACTTTTCACTTGTTTCAGAGCTAT | 142 |
| not | GCTGGAAACAGCATAGCAAGTTGAAATAAGGCTAGTCCGTTAT | |
| provided | CAACTTGAAAAAGTGGCACCGAGTCGGTGCTAGGCCCCAAGT | |
| | GAAAAGTTATTAAAAAAAAAAGCTGCCATCAGTCGGCGTGGAC | |
| | TGTAGAACACTGCCAATGCCGGTCCCAAGCCCGGATAAAAGT | |
| | GGAGGGTACAGTCCACGCTTTTTTTGGGGGAGAAGCTTTGGC | |
| | TTCTCTGATAAGCAGTACTGTAGGCCCTAAGTGAAAAGTTATTA | |
| | TCACTGTTAAATCATCATGgctaagcAGCTTGGCGTAACTAGATC | |
| | T | |
| Phenylketonuria; | ggccaaagcatgcatGTGATAATAACTTTTCACTTGTTTCAGAGCTAT | 143 |
| not | GCTGGAAACAGCATAGCAAGTTGAAATAAGGCTAGTCCGTTAT | |
| provided | CAACTTGAAAAAGTGGCACCGAGTCGGTGCCTGTAGGCCCCA | |
| | AGTGAAAAGTTATTAAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGGAGAAGCTTTGGCT | |
| | TCTCTGATAAGCAGTACTGTAGGCCCTAAGTGAAAAGTTATTAT | |
| | CACTGTTAAATCCCGGTGgctaagcAGCTTGGCGTAACTAGATCT | |
| Phenylketonuria; | ggccaaagcatgcatGTGATAATAACTTTTCACTTGTTTCAGAGCTAT | 144 |
| not | GCTGGAAACAGCATAGCAAGTTGAAATAAGGCTAGTCCGTTAT | |
| provided | CAACTTGAAAAAGTGGCACCGAGTCGGTGCCAGTACTGTAGG | |
| | CCCCAAGTGAAAAGTTATTAAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTAGCTTTGGCT | |
| | TCTCTGATAAGCAGTACTGTAGGCCCTAAGTGAAAAGTTATTAT | |
| | CACTGTTAAATCCCTTCAgctaagcAGCTTGGCGTAACTAGATCT | |
| Phenylketonuria; | ggccaaagcatgcatGGATAATAACTTTTCACTTAGTTTCAGAGCTAT | 145 |
| not | GCTGGAAACAGCATAGCAAGTTGAAATAAGGCTAGTCCGTTAT | |
| provided | CAACTTGAAAAAGTGGCACCGAGTCGGTGCCCCCAAGTGAAA | |
| | AGTTATTAAAAAAAAAGCTGCCATCAGTCGGCGTGGACTGTAG | |
| | AACACTGCCAATGCCGGTCCCAAGCCCGGATAAAAGTGGAGG | |
| | GTACAGTCCACGCTTTTTTTGCTCCAGGGGGAGAAGCTTTGGC | |
| | TTCTCTGATAAGCAGTACTGTAGGCCCTAAGTGAAAAGTTATTA | |
| | TCACTGTTAAATGGGACAgctaagcAGCTTGGCGTAACTAGATCT | |
| Phenylketonuria; | ggccaaagcatgcatGGATAATAACTTTTCACTTAGTTTCAGAGCTAT | 146 |
| not | GCTGGAAACAGCATAGCAAGTTGAAATAAGGCTAGTCCGTTAT | |
| provided | CAACTTGAAAAAGTGGCACCGAGTCGGTGCTAGGCCCCAAGT | |
| | GAAAAGTTATTAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTCAGGGGGAGAAGCTTTGG | |
| | CTTCTCTGATAAGCAGTACTGTAGGCCCTAAGTGAAAAGTTAT | |
| | TATCACTGTTAAATCCATGAgctaagcAGCTTGGCGTAACTAGAT | |
| | CT | |
| Phenylketonuria; | ggccaaagcatgcatGGATAATAACTTTTCACTTAGTTTCAGAGCTAT | 147 |
| not | GCTGGAAACAGCATAGCAAGTTGAAATAAGGCTAGTCCGTTAT | |
| provided | CAACTTGAAAAAGTGGCACCGAGTCGGTGCCTGTAGGCCCCA | |
| | AGTGAAAAGTTATTAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTGGGGAGAAGCTTTGG | |
| | CTTCTCTGATAAGCAGTACTGTAGGCCCTAAGTGAAAAGTTAT | |
| | TATCACTGTTAAATTCTTATgctaagcAGCTTGGCGTAACTAGATC | |
| | T | |
| Phenylketonuria; | ggccaaagcatgcatGGATAATAACTTTTCACTTAGTTTCAGAGCTAT | 148 |
| not | GCTGGAAACAGCATAGCAAGTTGAAATAAGGCTAGTCCGTTAT | |
| provided | CAACTTGAAAAAGTGGCACCGAGTCGGTGCCAGTACTGTAGG | |
| | CCCCAAGTGAAAAGTTATTAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTGAAGCTTTGGC | |
| | TTCTCTGATAAGCAGTACTGTAGGCCCTAAGTGAAAAGTTATTA | |
| | TCACTGTTAAATTGTCCTgctaagcAGCTTGGCGTAACTAGATCT | |
| Hyperphenyl | ggccaaagcatgcatGTTCCAGCCCCTCTATTACGGTTTCAGAGCTA | 149 |
| alaninemia, | TGCTGGAAACAGCATAGCAAGTTGAAATAAGGCTAGTCCGTTA | |
| non- | TCAACTTGAAAAAGTGGCACCGAGTCGGTGCCTCTCTGCCAC | |
| pku; | GTAATAGAGGGGCTGAAAAAAAAAGCTGCCATCAGTCGGCGT | |
| Phenylketonuria; | GGACTGTAGAACACTGCCAATGCCGGTCCCAAGCCCGGATAA | |
| not provided | AAGTGGAGGGTACAGTCCACGCTTTTTTTCCTCACCTTACTTT | |
| | CTCCTTGGCATCATTAAAACTCCCTGCCACGTAATAGAGGGGC | |
| | TGGAACTCCGTGACAATTGACgctaagcAGCTTGGCGTAACTAGA | |
| | TCT | |
| Hyperphenyl | ggccaaagcatgcatGTTCCAGCCCCTCTATTACGGTTTCAGAGCTA | 150 |
| alaninemia, | TGCTGGAAACAGCATAGCAAGTTGAAATAAGGCTAGTCCGTTA | |
| non- | TCAACTTGAAAAAGTGGCACCGAGTCGGTGCAAAACTCTCTGC | |
| pku; | CACGTAATAGAGGGGCTGAAAAAAAAAGCTGCCATCAGTCGG | |
| Phenylketonuria; | CGTGGACTGTAGAACACTGCCAATGCCGGTCCCAAGCCCGGA | |
| not provided | TAAAAGTGGAGGGTACAGTCCACGCTTTTTTTACCTTACTTTCT | |
| | CCTTGGCATCATTAAAACTCCCTGCCACGTAATAGAGGGGCTG | |
| | GAACTCCGTGACAGGCCACgctaagcAGCTTGGCGTAACTAGAT | |
| | CT | |
| Hyperphenyl | ggccaaagcatgcatGTTCCAGCCCCTCTATTACGGTTTCAGAGCTA | 151 |
| alaninemia, | TGCTGGAAACAGCATAGCAAGTTGAAATAAGGCTAGTCCGTTA | |
| non- | TCAACTTGAAAAAGTGGCACCGAGTCGGTGCATTAAAACTCTC | |
| pku; | TGCCACGTAATAGAGGGGCTGAAAAAAAAAGCTGCCATCAGT | |
| Phenylketonuria; | CGGCGTGGACTGTAGAACACTGCCAATGCCGGTCCCAAGCCC | |
| not provided | GGATAAAAGTGGAGGGTACAGTCCACGCTTTTTTTTTACTTTCT | |
| | CCTTGGCATCATTAAAACTCCCTGCCACGTAATAGAGGGGCTG | |
| | GAACTCCGTGACACCTGTAgctaagcAGCTTGGCGTAACTAGATC | |
| | T | |
| Hyperphenyl | ggccaaagcatgcatGTTCCAGCCCCTCTATTACGGTTTCAGAGCTA | 152 |
| alaninemia, | TGCTGGAAACAGCATAGCAAGTTGAAATAAGGCTAGTCCGTTA | |
| non- | TCAACTTGAAAAAGTGGCACCGAGTCGGTGCGCATCATTAAAA | |
| pku; | CTCTCTGCCACGTAATAGAGGGGCTGAAAAAAAAAGCTGCCAT | |
| Phenylketonuria; | CAGTCGGCGTGGACTGTAGAACACTGCCAATGCCGGTCCCAA | |
| not provided | GCCCGGATAAAAGTGGAGGGTACAGTCCACGCTTTTTTTTTCT | |
| | CCTTGGCATCATTAAAACTCCCTGCCACGTAATAGAGGGGCTG | |
| | GAACTCCGTGACAGAACATgctaagcAGCTTGGCGTAACTAGATC | |
| | T | |
| Hyperphenyl | ggccaaagcatgcatGAGCCCCTCTATTACGTGGCGTTTCAGAGCTA | 153 |
| alaninemia, | TGCTGGAAACAGCATAGCAAGTTGAAATAAGGCTAGTCCGTTA | |
| non- | TCAACTTGAAAAAGTGGCACCGAGTCGGTGCCTCTCTGCCAC | |
| pku; | GTAATAGAGGGAAAAAAAAAGCTGCCATCAGTCGGCGTGGAC | |
| Phenylketonuria; | TGTAGAACACTGCCAATGCCGGTCCCAAGCCCGGATAAAAGT | |
| not provided | GGAGGGTACAGTCCACGCTTTTTTTTGTCACCACCTCACCTTA | |
| | CTTTCTCCTTGGCATCATTAAAACTCCCTGCCACGTAATAGAG | |
| | GGGCTGGAACTCCGTGTAACCgctaagcAGCTTGGCGTAACTAG | |
| | ATCT | |
| Hyperphenyl | ggccaaagcatgcatGAGCCCCTCTATTACGTGGCGTTTCAGAGCTA | 154 |
| alaninemia, | TGCTGGAAACAGCATAGCAAGTTGAAATAAGGCTAGTCCGTTA | |
| non- | TCAACTTGAAAAAGTGGCACCGAGTCGGTGCAAAACTCTCTGC | |
| pku; | CACGTAATAGAGGGAAAAAAAAAGCTGCCATCAGTCGGCGTG | |
| Phenylketonuria; | GACTGTAGAACACTGCCAATGCCGGTCCCAAGCCCGGATAAA | |
| not provided | AGTGGAGGGTACAGTCCACGCTTTTTTTACCACCTCACCTTAC | |
| | TTTCTCCTTGGCATCATTAAAACTCCCTGCCACGTAATAGAGG | |
| | GGCTGGAACTCCGTGGACGTgctaagcAGCTTGGCGTAACTAGA | |
| | TCT | |
| Hyperphenyl | ggccaaagcatgcatGAGCCCCTCTATTACGTGGCGTTTCAGAGCTA | 155 |
| alaninemia, | TGCTGGAAACAGCATAGCAAGTTGAAATAAGGCTAGTCCGTTA | |
| non- | TCAACTTGAAAAAGTGGCACCGAGTCGGTGCATTAAAACTCTC | |
| pku; | TGCCACGTAATAGAGGGAAAAAAAAAGCTGCCATCAGTCGGC | |
| Phenylketonuria; | GTGGACTGTAGAACACTGCCAATGCCGGTCCCAAGCCCGGAT | |
| not provided | AAAAGTGGAGGGTACAGTCCACGCTTTTTTTACCTCACCTTAC | |
| | TTTCTCCTTGGCATCATTAAAACTCCCTGCCACGTAATAGAGG | |
| | GGCTGGAACTCCGTAAATAGgctaagcAGCTTGGCGTAACTAGAT | |
| | CT | |
| Hyperphenyl | ggccaaagcatgcatGAGCCCCTCTATTACGTGGCGTTTCAGAGCTA | 156 |
| alaninemia, | TGCTGGAAACAGCATAGCAAGTTGAAATAAGGCTAGTCCGTTA | |
| non- | TCAACTTGAAAAAGTGGCACCGAGTCGGTGCGCATCATTAAAA | |
| pku; | CTCTCTGCCACGTAATAGAGGGAAAAAAAAAGCTGCCATCAGT | |
| Phenylketonuria; | CGGCGTGGACTGTAGAACACTGCCAATGCCGGTCCCAAGCCC | |
| not provided | GGATAAAAGTGGAGGGTACAGTCCACGCTTTTTTTACCTTACT | |
| | TTCTCCTTGGCATCATTAAAACTCCCTGCCACGTAATAGAGGG | |
| | GCTGGAACTCCGTTGCGATgctaagcAGCTTGGCGTAACTAGAT | |
| | CT | |
| Hyperphenyl | ggccaaagcatgcatGGCCCCTCTATTACGTGGCAGTTTCAGAGCTA | 157 |
| alaninemia, | TGCTGGAAACAGCATAGCAAGTTGAAATAAGGCTAGTCCGTTA | |
| non- | TCAACTTGAAAAAGTGGCACCGAGTCGGTGCCTCTCTGCCAC | |
| pku; | GTAATAGAGGAAAAAAAAAGCTGCCATCAGTCGGCGTGGACT | |
| Phenylketonuria; | GTAGAACACTGCCAATGCCGGTCCCAAGCCCGGATAAAAGTG | |
| not provided | GAGGGTACAGTCCACGCTTTTTTTTTTGTCACCACCTCACCTTA | |
| | CTTTCTCCTTGGCATCATTAAAACTCCCTGCCACGTAATAGAG | |
| | GGGCTGGAACTCCGTACATTgctaagcAGCTTGGCGTAACTAGAT | |
| | CT | |
| Hyperphenyl | ggccaaagcatgcatGGCCCCTCTATTACGTGGCAGTTTCAGAGCTA | 158 |
| alaninemia, | TGCTGGAAACAGCATAGCAAGTTGAAATAAGGCTAGTCCGTTA | |
| non- | TCAACTTGAAAAAGTGGCACCGAGTCGGTGCAAAACTCTCTGC | |
| pku; | CACGTAATAGAGGAAAAAAAAAGCTGCCATCAGTCGGCGTGG | |
| Phenylketonuria; | ACTGTAGAACACTGCCAATGCCGGTCCCAAGCCCGGATAAAA | |
| not provided | GTGGAGGGTACAGTCCACGCTTTTTTTTCACCACCTCACCTTA | |
| | CTTTCTCCTTGGCATCATTAAAACTCCCTGCCACGTAATAGAG | |
| | GGGCTGGAACTCCGACTACCgctaagcAGCTTGGCGTAACTAGA | |
| | TCT | |
| Hyperphenyl | ggccaaagcatgcatGGCCCCTCTATTACGTGGCAGTTTCAGAGCTA | 159 |
| alaninemia, | TGCTGGAAACAGCATAGCAAGTTGAAATAAGGCTAGTCCGTTA | |
| non- | TCAACTTGAAAAAGTGGCACCGAGTCGGTGCATTAAAACTCTC | |
| pku; | TGCCACGTAATAGAGGAAAAAAAAAGCTGCCATCAGTCGGCG | |
| Phenylketonuria; | TGGACTGTAGAACACTGCCAATGCCGGTCCCAAGCCCGGATA | |
| not provided | AAAGTGGAGGGTACAGTCCACGCTTTTTTTCCACCTCACCTTA | |
| | CTTTCTCCTTGGCATCATTAAAACTCCCTGCCACGTAATAGAG | |
| | GGGCTGGAACTCCGGGCGACgctaagcAGCTTGGCGTAACTAGA | |
| | TCT | |
| Hyperphenyl | ggccaaagcatgcatGGCCCCTCTATTACGTGGCAGTTTCAGAGCTA | 160 |
| alaninemia, | TGCTGGAAACAGCATAGCAAGTTGAAATAAGGCTAGTCCGTTA | |
| non- | TCAACTTGAAAAAGTGGCACCGAGTCGGTGCGCATCATTAAAA | |
| pku; | CTCTCTGCCACGTAATAGAGGAAAAAAAAAGCTGCCATCAGTC | |
| Phenylketonuria; | GGCGTGGACTGTAGAACACTGCCAATGCCGGTCCCAAGCCCG | |
| not provided | GATAAAAGTGGAGGGTACAGTCCACGCTTTTTTTTCACCTTAC | |
| | TTTCTCCTTGGCATCATTAAAACTCCCTGCCACGTAATAGAGG | |
| | GGCTGGAACTCCGACCCGCgctaagcAGCTTGGCGTAACTAGAT | |
| | CT | |
| Phenylketonuria; | ggccaaagcatgcatGCCTCCATGTATTCCACTCAGTTTCAGAGCTA | 161 |
| not | TGCTGGAAACAGCATAGCAAGTTGAAATAAGGCTAGTCCGTTA | |
| provided | TCAACTTGAAAAAGTGGCACCGAGTCGGTGCCCTCGAGTGGA | |
| | ATACATGGAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTTGCTTGAGACACCTATTTTGTGC | |
| | CTGTATTCTAGTGGGCAGCCCATCCCTTGAGTGGAATACATGG | |
| | AGGAAGAAAAGAACGTAACgctaagcAGCTTGGCGTAACTAGATC | |
| | T | |
| Phenylketonuria; | ggccaaagcatgcatGCCTCCATGTATTCCACTCAGTTTCAGAGCTA | 162 |
| not | TGCTGGAAACAGCATAGCAAGTTGAAATAAGGCTAGTCCGTTA | |
| provided | TCAACTTGAAAAAGTGGCACCGAGTCGGTGCCATCCCTCGAG | |
| | TGGAATACATGGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTGAGACACCTATTTTGT | |
| | GCCTGTATTCTAGTGGGCAGCCCATCCCTTGAGTGGAATACAT | |
| | GGAGGAAGAAAAGAACTAAAAgctaagcAGCTTGGCGTAACTAG | |
| | ATCT | |
| Phenylketonuria; | ggccaaagcatgcatGCCTCCATGTATTCCACTCAGTTTCAGAGCTA | 163 |
| not | TGCTGGAAACAGCATAGCAAGTTGAAATAAGGCTAGTCCGTTA | |
| provided | TCAACTTGAAAAAGTGGCACCGAGTCGGTGCGCCCATCCCTC | |
| | GAGTGGAATACATGGAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTGACACCTATTTTGT | |
| | GCCTGTATTCTAGTGGGCAGCCCATCCCTTGAGTGGAATACAT | |
| | GGAGGAAGAAAAGAATAAAAAgctaagcAGCTTGGCGTAACTAGA | |
| | TCT | |
| Phenylketonuria; | ggccaaagcatgcatGCCTCCATGTATTCCACTCAGTTTCAGAGCTA | 164 |
| not | TGCTGGAAACAGCATAGCAAGTTGAAATAAGGCTAGTCCGTTA | |
| provided | TCAACTTGAAAAAGTGGCACCGAGTCGGTGCGGGCAGCCCAT | |
| | CCCTCGAGTGGAATACATGGAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTCTATTTTGTGC | |
| | CTGTATTCTAGTGGGCAGCCCATCCCTTGAGTGGAATACATGG | |
| | AGGAAGAAAAGAATAGAGTgctaagcAGCTTGGCGTAACTAGATC | |
| | T | |
| Phenylketonuria; | ggccaaagcatgcatGCTCCATGTATTCCACTCAAGTTTCAGAGCTA | 165 |
| not | TGCTGGAAACAGCATAGCAAGTTGAAATAAGGCTAGTCCGTTA | |
| provided | TCAACTTGAAAAAGTGGCACCGAGTCGGTGCCCTCGAGTGGA | |
| | ATACATGAAAAAAAAAGCTGCCATCAGTCGGCGTGGACTGTAG | |
| | AACACTGCCAATGCCGGTCCCAAGCCCGGATAAAAGTGGAGG | |
| | GTACAGTCCACGCTTTTTTTCCTGCTTGAGACACCTATTTTGTG | |
| | CCTGTATTCTAGTGGGCAGCCCATCCCTTGAGTGGAATACATG | |
| | GAGGAAGAAAAGATCTCGTgctaagcAGCTTGGCGTAACTAGATC | |
| | T | |
| Phenylketonuria; | ggccaaagcatgcatGCTCCATGTATTCCACTCAAGTTTCAGAGCTA | 166 |
| not | TGCTGGAAACAGCATAGCAAGTTGAAATAAGGCTAGTCCGTTA | |
| provided | TCAACTTGAAAAAGTGGCACCGAGTCGGTGCCATCCCTCGAG | |
| | TGGAATACATGAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTCTTGAGACACCTATTTTGT | |
| | GCCTGTATTCTAGTGGGCAGCCCATCCCTTGAGTGGAATACAT | |
| | GGAGGAAGAAAAGATGTGCAgctaagcAGCTTGGCGTAACTAGA | |
| | TCT | |
| Phenylketonuria; | ggccaaagcatgcatGCTCCATGTATTCCACTCAAGTTTCAGAGCTA | 167 |
| not | TGCTGGAAACAGCATAGCAAGTTGAAATAAGGCTAGTCCGTTA | |
| provided | TCAACTTGAAAAAGTGGCACCGAGTCGGTGCGCCCATCCCTC | |
| | GAGTGGAATACATGAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGAGACACCTATTTTG | |
| | TGCCTGTATTCTAGTGGGCAGCCCATCCCTTGAGTGGAATACA | |
| | TGGAGGAAGAAAAGACACGGCgctaagcAGCTTGGCGTAACTAG | |
| | ATCT | |
| Phenylketonuria; | ggccaaagcatgcatGCTCCATGTATTCCACTCAAGTTTCAGAGCTA | 168 |
| not | TGCTGGAAACAGCATAGCAAGTTGAAATAAGGCTAGTCCGTTA | |
| provided | TCAACTTGAAAAAGTGGCACCGAGTCGGTGCGGGCAGCCCAT | |
| | CCCTCGAGTGGAATACATGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTACCTATTTTGTG | |
| | CCTGTATTCTAGTGGGCAGCCCATCCCTTGAGTGGAATACATG | |
| | GAGGAAGAAAAGAGGGGCAgctaagcAGCTTGGCGTAACTAGAT | |
| | CT | |
| Inborn | ggccaaagcatgcatGAGGAAAGCAGGCCAGCCACGTTTCAGAGCT | 169 |
| genetic | ATGCTGGAAACAGCATAGCAAGTTGAAATAAGGCTAGTCCGTT | |
| diseases; | ATCAACTTGAAAAAGTGGCACCGAGTCGGTGCTCCGACCTGT | |
| Phenylketonuria; | GGCTGGCCTGCTTTAAAAAAAAAGCTGCCATCAGTCGGCGTG | |
| not | GACTGTAGAACACTGCCAATGCCGGTCCCAAGCCCGGATAAA | |
| provided | AGTGGAGGGTACAGTCCACGCTTTTTTTTTTCTTCTTTTCATCC | |
| | CAGCTTGCACTGGTTTCCGCCTCCAACCTGTGGCTGGCCTGC | |
| | TTTCCTCTCGGGATTTGATATGgctaagcAGCTTGGCGTAACTAG | |
| | ATCT | |
| Inborn | ggccaaagcatgcatGAGGAAAGCAGGCCAGCCACGTTTCAGAGCT | 170 |
| genetic | ATGCTGGAAACAGCATAGCAAGTTGAAATAAGGCTAGTCCGTT | |
| diseases; | ATCAACTTGAAAAAGTGGCACCGAGTCGGTGCCTGGTTTCCG | |
| Phenylketonuria; | CCTCCGACCTGTGGCTGGCCTGCTTTAAAAAAAAAGCTGCCAT | |
| not | CAGTCGGCGTGGACTGTAGAACACTGCCAATGCCGGTCCCAA | |
| provided | GCCCGGATAAAAGTGGAGGGTACAGTCCACGCTTTTTTTATCC | |
| | CAGCTTGCACTGGTTTCCGCCTCCAACCTGTGGCTGGCCTGC | |
| | TTTCCTCTCGGGATTTTCGAGGgctaagcAGCTTGGCGTAACTAG | |
| | ATCT | |
| Inborn | ggccaaagcatgcatGAAGCAGGCCAGCCACAGGTGTTTCAGAGCT | 171 |
| genetic | ATGCTGGAAACAGCATAGCAAGTTGAAATAAGGCTAGTCCGTT | |
| diseases; | ATCAACTTGAAAAAGTGGCACCGAGTCGGTGCTCCGACCTGT | |
| Phenylketonuria; | GGCTGGCCTGAAAAAAAAAGCTGCCATCAGTCGGCGTGGACT | |
| not | GTAGAACACTGCCAATGCCGGTCCCAAGCCCGGATAAAAGTG | |
| provided | GAGGGTACAGTCCACGCTTTTTTTGAGTTTTCTTTCTTCTTTTC | |
| | ATCCCAGCTTGCACTGGTTTCCGCCTCCAACCTGTGGCTGGC | |
| | CTGCTTTCCTCTCGGGTCAATTgctaagcAGCTTGGCGTAACTAG | |
| | ATCT | |
| Inborn | ggccaaagcatgcatGAAGCAGGCCAGCCACAGGTGTTTCAGAGCT | 172 |
| genetic | ATGCTGGAAACAGCATAGCAAGTTGAAATAAGGCTAGTCCGTT | |
| diseases;Ph | ATCAACTTGAAAAAGTGGCACCGAGTCGGTGCCGCCTCCGAC | |
| diseases; | CTGTGGCTGGCCTGAAAAAAAAAGCTGCCATCAGTCGGCGTG | |
| Phenylketonuria; | GACTGTAGAACACTGCCAATGCCGGTCCCAAGCCCGGATAAA | |
| not | AGTGGAGGGTACAGTCCACGCTTTTTTTTTTCTTTCTTCTTTTC | |
| | ATCCCAGCTTGCACTGGTTTCCGCCTCCAACCTGTGGCTGGC | |
| | CTGCTTTCCTCTCGGGTAGTTTgctaagcAGCTTGGCGTAACTAG | |
| | ATCT | |
| Inborn | ggccaaagcatgcatGAAGCAGGCCAGCCACAGGTGTTTCAGAGCT | 173 |
| genetic | ATGCTGGAAACAGCATAGCAAGTTGAAATAAGGCTAGTCCGTT | |
| diseases; | ATCAACTTGAAAAAGTGGCACCGAGTCGGTGCCTGGTTTCCG | |
| Phenylketonuria; | CCTCCGACCTGTGGCTGGCCTGAAAAAAAAAGCTGCCATCAG | |
| not | TCGGCGTGGACTGTAGAACACTGCCAATGCCGGTCCCAAGCC | |
| provided | CGGATAAAAGTGGAGGGTACAGTCCACGCTTTTTTTTTCTTTTC | |
| | ATCCCAGCTTGCACTGGTTTCCGCCTCCAACCTGTGGCTGGC | |
| | CTGCTTTCCTCTCGGGTGTAGCgctaagcAGCTTGGCGTAACTAG | |
| | ATCT | |
| Inborn | ggccaaagcatgcatGCAGGCCAGCCACAGGTTGGGTTTCAGAGCT | 174 |
| genetic | ATGCTGGAAACAGCATAGCAAGTTGAAATAAGGCTAGTCCGTT | |
| diseases; | ATCAACTTGAAAAAGTGGCACCGAGTCGGTGCTCCGACCTGT | |
| Phenylketonuria; | GGCTGGCAAAAAAAAAGCTGCCATCAGTCGGCGTGGACTGTA | |
| not | GAACACTGCCAATGCCGGTCCCAAGCCCGGATAAAAGTGGAG | |
| provided | GGTACAGTCCACGCTTTTTTTAGCTTTGAGTTTTCTTTCTTCTTT | |
| | TCATCCCAGCTTGCACTGGTTTCCGCCTCCAACCTGTGGCTG | |
| | GCCTGCTTTCCTCTCGTACATgctaagcAGCTTGGCGTAACTAGA | |
| | TCT | |
| Inborn | ggccaaagcatgcatGCAGGCCAGCCACAGGTTGGGTTTCAGAGCT | 175 |
| genetic | ATGCTGGAAACAGCATAGCAAGTTGAAATAAGGCTAGTCCGTT | |
| diseases; | ATCAACTTGAAAAAGTGGCACCGAGTCGGTGCCTGGTTTCCG | |
| Phenylketonuria; | CCTCCGACCTGTGGCTGGCAAAAAAAAAGCTGCCATCAGTCG | |
| not | GCGTGGACTGTAGAACACTGCCAATGCCGGTCCCAAGCCCGG | |
| provided | ATAAAAGTGGAGGGTACAGTCCACGCTTTTTTTTCTTTCTTCTT | |
| | TTCATCCCAGCTTGCACTGGTTTCCGCCTCCAACCTGTGGCTG | |
| | GCCTGCTTTCCTCTCTTTTACgctaagcAGCTTGGCGTAACTAGA | |
| | TCT | |
| Hyperphenyl | ggccaaagcatgcatGCCTCCATGTATTCCACTAGGTTTCAGAGCTA | 176 |
| alaninemia, | TGCTGGAAACAGCATAGCAAGTTGAAATAAGGCTAGTCCGTTA | |
| non- | TCAACTTGAAAAAGTGGCACCGAGTCGGTGCCTCGAGTGGAA | |
| pku; | TACATGGAAAAAAAAAGCTGCCATCAGTCGGCGTGGACTGTA | |
| Phenylketonuria; | GAACACTGCCAATGCCGGTCCCAAGCCCGGATAAAAGTGGAG | |
| not provided | GGTACAGTCCACGCTTTTTTTCTGCTTGAGACACCTATTTTGTG | |
| | CCTGTATTCTAGTGGGCAGCCCATCCCTCTAGTGGAATACATG | |
| | GAGGAAGAAAAGAAGTCCATgctaagcAGCTTGGCGTAACTAGAT | |
| | CT | |
| Hyperphenyl | ggccaaagcatgcatGCCTCCATGTATTCCACTAGGTTTCAGAGCTA | 177 |
| alaninemia, | TGCTGGAAACAGCATAGCAAGTTGAAATAAGGCTAGTCCGTTA | |
| non- | TCAACTTGAAAAAGTGGCACCGAGTCGGTGCATCCCTCGAGT | |
| pku; | GGAATACATGGAAAAAAAAAGCTGCCATCAGTCGGCGTGGAC | |
| Phenylketonuria; | TGTAGAACACTGCCAATGCCGGTCCCAAGCCCGGATAAAAGT | |
| not provided | GGAGGGTACAGTCCACGCTTTTTTTTTGAGACACCTATTTTGT | |
| | GCCTGTATTCTAGTGGGCAGCCCATCCCTCTAGTGGAATACAT | |
| | GGAGGAAGAAAAGAACGATTTgctaagcAGCTTGGCGTAACTAG | |
| | ATCT | |
| Hyperphenyl | ggccaaagcatgcatGCCTCCATGTATTCCACTAGGTTTCAGAGCTA | 178 |
| alaninemia, | TGCTGGAAACAGCATAGCAAGTTGAAATAAGGCTAGTCCGTTA | |
| non- | TCAACTTGAAAAAGTGGCACCGAGTCGGTGCCCCATCCCTCG | |
| pku; | AGTGGAATACATGGAAAAAAAAAGCTGCCATCAGTCGGCGTG | |
| Phenylketonuria; | GACTGTAGAACACTGCCAATGCCGGTCCCAAGCCCGGATAAA | |
| not provided | AGTGGAGGGTACAGTCCACGCTTTTTTTAGACACCTATTTTGT | |
| | GCCTGTATTCTAGTGGGCAGCCCATCCCTCTAGTGGAATACAT | |
| | GGAGGAAGAAAAGAATTAGTCgctaagcAGCTTGGCGTAACTAG | |
| | ATCT | |
| Hyperphenyl | ggccaaagcatgcatGCCTCCATGTATTCCACTAGGTTTCAGAGCTA | 179 |
| alaninemia, | TGCTGGAAACAGCATAGCAAGTTGAAATAAGGCTAGTCCGTTA | |
| non- | TCAACTTGAAAAAGTGGCACCGAGTCGGTGCGGCAGCCCATC | |
| pku; | CCTCGAGTGGAATACATGGAAAAAAAAAGCTGCCATCAGTCG | |
| Phenylketonuria; | GCGTGGACTGTAGAACACTGCCAATGCCGGTCCCAAGCCCGG | |
| not provided | ATAAAAGTGGAGGGTACAGTCCACGCTTTTTTTCCTATTTTGTG | |
| | CCTGTATTCTAGTGGGCAGCCCATCCCTCTAGTGGAATACATG | |
| | GAGGAAGAAAAGAAGGATGCgctaagcAGCTTGGCGTAACTAGA | |
| | TCT | |
| Hyperphenyl | ggccaaagcatgcatGCTCCATGTATTCCACTAGAGTTTCAGAGCTA | 180 |
| alaninemia, | TGCTGGAAACAGCATAGCAAGTTGAAATAAGGCTAGTCCGTTA | |
| non- | TCAACTTGAAAAAGTGGCACCGAGTCGGTGCCTCGAGTGGAA | |
| pku; | TACATGAAAAAAAAAGCTGCCATCAGTCGGCGTGGACTGTAGA | |
| Phenylketonuria; | ACACTGCCAATGCCGGTCCCAAGCCCGGATAAAAGTGGAGGG | |
| not provided | TACAGTCCACGCTTTTTTTCCCTGCTTGAGACACCTATTTTGTG | |
| | CCTGTATTCTAGTGGGCAGCCCATCCCTCTAGTGGAATACATG | |
| | GAGGAAGAAAAGAACCCATgctaagcAGCTTGGCGTAACTAGAT | |
| | CT | |
| Hyperphenyl | ggccaaagcatgcatGCTCCATGTATTCCACTAGAGTTTCAGAGCTA | 181 |
| alaninemia, | TGCTGGAAACAGCATAGCAAGTTGAAATAAGGCTAGTCCGTTA | |
| non- | TCAACTTGAAAAAGTGGCACCGAGTCGGTGCATCCCTCGAGT | |
| pku; | GGAATACATGAAAAAAAAAGCTGCCATCAGTCGGCGTGGACT | |
| Phenylketonuria; | GTAGAACACTGCCAATGCCGGTCCCAAGCCCGGATAAAAGTG | |
| not provided | GAGGGTACAGTCCACGCTTTTTTTGCTTGAGACACCTATTTTG | |
| | TGCCTGTATTCTAGTGGGCAGCCCATCCCTCTAGTGGAATACA | |
| | TGGAGGAAGAAAAGACTACCAgctaagcAGCTTGGCGTAACTAG | |
| | ATCT | |
| Hyperphenyl | ggccaaagcatgcatGCTCCATGTATTCCACTAGAGTTTCAGAGCTA | 182 |
| alaninemia, | TGCTGGAAACAGCATAGCAAGTTGAAATAAGGCTAGTCCGTTA | |
| non- | TCAACTTGAAAAAGTGGCACCGAGTCGGTGCCCCATCCCTCG | |
| pku;Phenylk | AGTGGAATACATGAAAAAAAAAGCTGCCATCAGTCGGCGTGG | |
| etonuria;not | ACTGTAGAACACTGCCAATGCCGGTCCCAAGCCCGGATAAAA | |
| provided | GTGGAGGGTACAGTCCACGCTTTTTTTTGAGACACCTATTTTG | |
| | TGCCTGTATTCTAGTGGGCAGCCCATCCCTCTAGTGGAATACA | |
| | TGGAGGAAGAAAAGAGGGATAgctaagcAGCTTGGCGTAACTAG | |
| | ATCT | |
| Hyperphenyl | ggccaaagcatgcatGCTCCATGTATTCCACTAGAGTTTCAGAGCTA | 183 |
| alaninemia, | TGCTGGAAACAGCATAGCAAGTTGAAATAAGGCTAGTCCGTTA | |
| non- | TCAACTTGAAAAAGTGGCACCGAGTCGGTGCGGCAGCCCATC | |
| pku; | CCTCGAGTGGAATACATGAAAAAAAAAGCTGCCATCAGTCGGC | |
| Phenylketonuria; | GTGGACTGTAGAACACTGCCAATGCCGGTCCCAAGCCCGGAT | |
| not provided | AAAAGTGGAGGGTACAGTCCACGCTTTTTTTCACCTATTTTGT | |
| | GCCTGTATTCTAGTGGGCAGCCCATCCCTCTAGTGGAATACAT | |
| | GGAGGAAGAAAAGAAACGACgctaagcAGCTTGGCGTAACTAGA | |
| | TCT | |
| Hyperphenyl | ggccaaagcatgcatGGGGCAGCCCATCCCTCTAGGTTTCAGAGCT | 184 |
| alaninemia, | ATGCTGGAAACAGCATAGCAAGTTGAAATAAGGCTAGTCCGTT | |
| non- | ATCAACTTGAAAAAGTGGCACCGAGTCGGTGCACTCGAGGGA | |
| pku; | TGGGCTGAAAAAAAAAGCTGCCATCAGTCGGCGTGGACTGTA | |
| Phenylketonuria; | GAACACTGCCAATGCCGGTCCCAAGCCCGGATAAAAGTGGAG | |
| not provided | GGTACAGTCCACGCTTTTTTTAGAGTCTTGAACACTGTGCCCC | |
| | ATGTTTTCTTTTCTTCCTCCATGTATTCCACTAGAGGGATGGGC | |
| | TGCCCACTAGAATACCAGGCCgctaagcAGCTTGGCGTAACTAG | |
| | ATCT | |
| Hyperphenyl | ggccaaagcatgcatGGGGCAGCCCATCCCTCTAGGTTTCAGAGCT | 185 |
| alaninemia, | ATGCTGGAAACAGCATAGCAAGTTGAAATAAGGCTAGTCCGTT | |
| non- | ATCAACTTGAAAAAGTGGCACCGAGTCGGTGCTTCCACTCGA | |
| pku; | GGGATGGGCTGAAAAAAAAAGCTGCCATCAGTCGGCGTGGAC | |
| Phenylketonuria; | TGTAGAACACTGCCAATGCCGGTCCCAAGCCCGGATAAAAGT | |
| not provided | GGAGGGTACAGTCCACGCTTTTTTTTCTTGAACACTGTGCCCC | |
| | ATGTTTTCTTTTCTTCCTCCATGTATTCCACTAGAGGGATGGGC | |
| | TGCCCACTAGAATACACTGGCgctaagcAGCTTGGCGTAACTAGA | |
| | TCT | |
| Hyperphenyl | ggccaaagcatgcatGGGGCAGCCCATCCCTCTAGGTTTCAGAGCT | 186 |
| alaninemia, | ATGCTGGAAACAGCATAGCAAGTTGAAATAAGGCTAGTCCGTT | |
| non- | ATCAACTTGAAAAAGTGGCACCGAGTCGGTGCGTATTCCACTC | |
| pku; | GAGGGATGGGCTGAAAAAAAAAGCTGCCATCAGTCGGCGTGG | |
| Phenylketonuria; | ACTGTAGAACACTGCCAATGCCGGTCCCAAGCCCGGATAAAA | |
| not provided | GTGGAGGGTACAGTCCACGCTTTTTTTTGAACACTGTGCCCCA | |
| | TGTTTTCTTTTCTTCCTCCATGTATTCCACTAGAGGGATGGGCT | |
| | GCCCACTAGAATACAGCTACgctaagcAGCTTGGCGTAACTAGAT | |
| | CT | |
| Hyperphenyl | ggccaaagcatgcatGGGGCAGCCCATCCCTCTAGGTTTCAGAGCT | 187 |
| alaninemia, | ATGCTGGAAACAGCATAGCAAGTTGAAATAAGGCTAGTCCGTT | |
| non- | ATCAACTTGAAAAAGTGGCACCGAGTCGGTGCTCCATGTATTC | |
| pku; | CACTCGAGGGATGGGCTGAAAAAAAAAGCTGCCATCAGTCGG | |
| Phenylketonuria; | CGTGGACTGTAGAACACTGCCAATGCCGGTCCCAAGCCCGGA | |
| not provided | TAAAAGTGGAGGGTACAGTCCACGCTTTTTTTACTGTGCCCCA | |
| | TGTTTTCTTTTCTTCCTCCATGTATTCCACTAGAGGGATGGGCT | |
| | GCCCACTAGAATACGCGCGGgctaagcAGCTTGGCGTAACTAGA | |
| | TCT | |
| Phenylketonuria; | ggccaaagcatgcatGCCACCCAAGAAATCCCAAGGTTTCAGAGCTA | 188 |
| Phenylketonuria; | TGCTGGAAACAGCATAGCAAGTTGAAATAAGGCTAGTCCGTTA | |
| not | TCAACTTGAAAAAGTGGCACCGAGTCGGTGCTCTCGGGATTTC | |
| provided | TTGGGAAAAAAAAAGCTGCCATCAGTCGGCGTGGACTGTAGA | |
| | ACACTGCCAATGCCGGTCCCAAGCCCGGATAAAAGTGGAGGG | |
| | TACAGTCCACGCTTTTTTTATCCCAGCTTGCACTGGTTTCCGC | |
| | CTCCGACCTGTGGCTGGCCTGCTTTCCTCTTGGGATTTCTTGG | |
| | GTGGCCTGGCCTTCGCCGAGgctaagcAGCTTGGCGTAACTAGA | |
| | TCT | |
| Phenylketonuria; | ggccaaagcatgcatGCCACCCAAGAAATCCCAAGGTTTCAGAGCTA | 189 |
| Phenylketonuria; | TGCTGGAAACAGCATAGCAAGTTGAAATAAGGCTAGTCCGTTA | |
| not | TCAACTTGAAAAAGTGGCACCGAGTCGGTGCTTCCTCTCGGG | |
| provided | ATTTCTTGGGAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTCAGCTTGCACTGGTTTCCGC | |
| | CTCCGACCTGTGGCTGGCCTGCTTTCCTCTTGGGATTTCTTGG | |
| | GTGGCCTGGCCTTCGTTCGAgctaagcAGCTTGGCGTAACTAGA | |
| | TCT | |
| Phenylketonuria; | ggccaaagcatgcatGCCACCCAAGAAATCCCAAGGTTTCAGAGCTA | 190 |
| Phenylketonuria; | TGCTGGAAACAGCATAGCAAGTTGAAATAAGGCTAGTCCGTTA | |
| not | TCAACTTGAAAAAGTGGCACCGAGTCGGTGCGCTTTCCTCTCG | |
| provided | GGATTTCTTGGGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTCTTGCACTGGTTTCCGC | |
| | CTCCGACCTGTGGCTGGCCTGCTTTCCTCTTGGGATTTCTTGG | |
| | GTGGCCTGGCCTTCGGCTCGgctaagcAGCTTGGCGTAACTAGA | |
| | TCT | |
| Phenylketonuria; | ggccaaagcatgcatGCCACCCAAGAAATCCCAAGGTTTCAGAGCTA | 191 |
| Phenylketonuria; | TGCTGGAAACAGCATAGCAAGTTGAAATAAGGCTAGTCCGTTA | |
| not | TCAACTTGAAAAAGTGGCACCGAGTCGGTGCGGCCTGCTTTC | |
| provided | CTCTCGGGATTTCTTGGGAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTACTGGTTTCCGC | |
| | CTCCGACCTGTGGCTGGCCTGCTTTCCTCTTGGGATTTCTTGG | |
| | GTGGCCTGGCCTTCTTTTTAgctaagcAGCTTGGCGTAACTAGAT | |
| | CT | |
| Phenylketonuria; | ggccaaagcatgcatGGCTGGCCTGCTTTCCTCTTGTTTCAGAGCTA | 192 |
| Phenylketonuria; | TGCTGGAAACAGCATAGCAAGTTGAAATAAGGCTAGTCCGTTA | |
| not | TCAACTTGAAAAAGTGGCACCGAGTCGGTGCCCCGAGAGGAA | |
| provided | AGCAGGCCAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTGTACTGTGTGCAGTGGAAGAC | |
| | TCGGAAGGCCAGGCCACCCAAGAAATCCCAAGAGGAAAGCAG | |
| | GCCAGCCACAGGTCGGGAATACgctaagcAGCTTGGCGTAACTA | |
| | GATCT | |
| Phenylketonuria; | ggccaaagcatgcatGGCTGGCCTGCTTTCCTCTTGTTTCAGAGCTA | 193 |
| Phenylketonuria; | TGCTGGAAACAGCATAGCAAGTTGAAATAAGGCTAGTCCGTTA | |
| not | TCAACTTGAAAAAGTGGCACCGAGTCGGTGCAAATCCCGAGA | |
| provided | GGAAAGCAGGCCAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTGTGTGCAGTGGAAGAC | |
| | TCGGAAGGCCAGGCCACCCAAGAAATCCCAAGAGGAAAGCAG | |
| | GCCAGCCACAGGTCGGAAACTAgctaagcAGCTTGGCGTAACTA | |
| | GATCT | |
| Phenylketonuria; | ggccaaagcatgcatGGAGGACAGTACTCACGGTTGTTTCAGAGCTA | 194 |
| not | TGCTGGAAACAGCATAGCAAGTTGAAATAAGGCTAGTCCGTTA | |
| provided | TCAACTTGAAAAAGTGGCACCGAGTCGGTGCATGTATACCCCC | |
| | GAACCGTGAGTACTGTCAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTCAGTACATCAGA | |
| | CATGGATCCAAGCCCATGGATACCCCCGAACCGTGAGTACTG | |
| | TCCTCCAGCTACCAGACCAAGgctaagcAGCTTGGCGTAACTAG | |
| | ATCT | |
| Phenylketonuria; | ggccaaagcatgcatGGAGGACAGTACTCACGGTTGTTTCAGAGCTA | 195 |
| not | TGCTGGAAACAGCATAGCAAGTTGAAATAAGGCTAGTCCGTTA | |
| provided | TCAACTTGAAAAAGTGGCACCGAGTCGGTGCGCCCATGTATA | |
| | CCCCCGAACCGTGAGTACTGTCAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTACATCAGA | |
| | CATGGATCCAAGCCCATGGATACCCCCGAACCGTGAGTACTG | |
| | TCCTCCAGCTACCAGCCACATgctaagcAGCTTGGCGTAACTAGA | |
| | TCT | |
| Phenylketonuria; | ggccaaagcatgcatGGAGGACAGTACTCACGGTTGTTTCAGAGCTA | 196 |
| not | TGCTGGAAACAGCATAGCAAGTTGAAATAAGGCTAGTCCGTTA | |
| provided | TCAACTTGAAAAAGTGGCACCGAGTCGGTGCCAAGCCCATGT | |
| | ATACCCCCGAACCGTGAGTACTGTCAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTTCAGA | |
| | CATGGATCCAAGCCCATGGATACCCCCGAACCGTGAGTACTG | |
| | TCCTCCAGCTACCAGTCCTAGgctaagcAGCTTGGCGTAACTAGA | |
| | TCT | |
| Phenylketonuria; | ggccaaagcatgcatGGAGGACAGTACTCACGGTTGTTTCAGAGCTA | 197 |
| not | TGCTGGAAACAGCATAGCAAGTTGAAATAAGGCTAGTCCGTTA | |
| provided | TCAACTTGAAAAAGTGGCACCGAGTCGGTGCGGATCCAAGCC | |
| | CATGTATACCCCCGAACCGTGAGTACTGTCAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | CATGGATCCAAGCCCATGGATACCCCCGAACCGTGAGTACTG | |
| | TCCTCCAGCTACCAGAGAATCgctaagcAGCTTGGCGTAACTAGA | |
| | TCT | |
| Phenylketonuria; | ggccaaagcatgcatGGGACAGTACTCACGGTTCGGTTTCAGAGCTA | 198 |
| not | TGCTGGAAACAGCATAGCAAGTTGAAATAAGGCTAGTCCGTTA | |
| provided | TCAACTTGAAAAAGTGGCACCGAGTCGGTGCGCCCATGTATA | |
| | CCCCCGAACCGTGAGTACTGAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCAGTACATC | |
| | AGACATGGATCCAAGCCCATGGATACCCCCGAACCGTGAGTA | |
| | CTGTCCTCCAGCTACCACCGAAgctaagcAGCTTGGCGTAACTA | |
| | GATCT | |
| Phenylketonuria; | ggccaaagcatgcatGGGACAGTACTCACGGTTCGGTTTCAGAGCTA | 199 |
| not | TGCTGGAAACAGCATAGCAAGTTGAAATAAGGCTAGTCCGTTA | |
| provided | TCAACTTGAAAAAGTGGCACCGAGTCGGTGCCAAGCCCATGT | |
| | ATACCCCCGAACCGTGAGTACTGAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTTACATCA | |
| | GACATGGATCCAAGCCCATGGATACCCCCGAACCGTGAGTAC | |
| | TGTCCTCCAGCTACCAGTGTCgctaagcAGCTTGGCGTAACTAGA | |
| | TCT | |
| Phenylketonuria; | ggccaaagcatgcatGGGACAGTACTCACGGTTCGGTTTCAGAGCTA | 200 |
| not | TGCTGGAAACAGCATAGCAAGTTGAAATAAGGCTAGTCCGTTA | |
| provided | TCAACTTGAAAAAGTGGCACCGAGTCGGTGCGGATCCAAGCC | |
| | CATGTATACCCCCGAACCGTGAGTACTGAAAAAAAAAGCTGCC | |
| | ATCAGTCGGCGTGGACTGTAGAACACTGCCAATGCCGGTCCC | |
| | AAGCCCGGATAAAAGTGGAGGGTACAGTCCACGCTTTTTTTCA | |
| | GACATGGATCCAAGCCCATGGATACCCCCGAACCGTGAGTAC | |
| | TGTCCTCCAGCTACCCGTTGAgctaagcAGCTTGGCGTAACTAGA | |
| | TCT | |
| Phenylketonuria; | ggccaaagcatgcatGGACAGTACTCACGGTTCGGGTTTCAGAGCTA | 201 |
| not | TGCTGGAAACAGCATAGCAAGTTGAAATAAGGCTAGTCCGTTA | |
| provided | TCAACTTGAAAAAGTGGCACCGAGTCGGTGCATGTATACCCCC | |
| | GAACCGTGAGTACTAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTGCACACAGTACATC | |
| | AGACATGGATCCAAGCCCATGGATACCCCCGAACCGTGAGTA | |
| | CTGTCCTCCAGCTACTCTTTCgctaagcAGCTTGGCGTAACTAGA | |
| | TCT | |
| Phenylketonuria; | ggccaaagcatgcatGGACAGTACTCACGGTTCGGGTTTCAGAGCTA | 202 |
| not | TGCTGGAAACAGCATAGCAAGTTGAAATAAGGCTAGTCCGTTA | |
| provided | TCAACTTGAAAAAGTGGCACCGAGTCGGTGCGCCCATGTATA | |
| | CCCCCGAACCGTGAGTACTAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTCACAGTACAT | |
| | CAGACATGGATCCAAGCCCATGGATACCCCCGAACCGTGAGT | |
| | ACTGTCCTCCAGCTACCCTAGGgctaagcAGCTTGGCGTAACTA | |
| | GATCT | |
| Phenylketonuria; | ggccaaagcatgcatGGACAGTACTCACGGTTCGGGTTTCAGAGCTA | 203 |
| not | TGCTGGAAACAGCATAGCAAGTTGAAATAAGGCTAGTCCGTTA | |
| provided | TCAACTTGAAAAAGTGGCACCGAGTCGGTGCCAAGCCCATGT | |
| | ATACCCCCGAACCGTGAGTACTAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTAGTACATC | |
| | AGACATGGATCCAAGCCCATGGATACCCCCGAACCGTGAGTA | |
| | CTGTCCTCCAGCTACAGTCTAgctaagcAGCTTGGCGTAACTAGA | |
| | TCT | |
| Phenylketonuria; | ggccaaagcatgcatGGACAGTACTCACGGTTCGGGTTTCAGAGCTA | 204 |
| not | TGCTGGAAACAGCATAGCAAGTTGAAATAAGGCTAGTCCGTTA | |
| provided | TCAACTTGAAAAAGTGGCACCGAGTCGGTGCGGATCCAAGCC | |
| | CATGTATACCCCCGAACCGTGAGTACTAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTATC | |
| | AGACATGGATCCAAGCCCATGGATACCCCCGAACCGTGAGTA | |
| | CTGTCCTCCAGCTACACTACGgctaagcAGCTTGGCGTAACTAGA | |
| | TCT | |
| Argininosuccinate | ggccaaagcatgcatGGGGAAGCAGCCTGATGCCCGTTTCAGAGCT | 205 |
| lyase | ATGCTGGAAACAGCATAGCAAGTTGAAATAAGGCTAGTCCGTT | |
| deficiency; | ATCAACTTGAAAAAGTGGCACCGAGTCGGTGCTTCTGGGGCA | |
| not provided | TCAGGCTGCTTAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTCCCAGCCTTGCTCCGGATC | |
| | AGCTCCAAACTGTCGGGGTTTTTCTTCCGGGGCATCAGGCTG | |
| | CTTCCCGTGCTGGGGAGGAGGAgctaagcAGCTTGGCGTAACTA | |
| | GATCT | |
| Argininosuccinate | ggccaaagcatgcatGGGGAAGCAGCCTGATGCCCGTTTCAGAGCT | 206 |
| lyase | ATGCTGGAAACAGCATAGCAAGTTGAAATAAGGCTAGTCCGTT | |
| deficiency; | ATCAACTTGAAAAAGTGGCACCGAGTCGGTGCTTTCTTCTGGG | |
| not provided | GCATCAGGCTGCTTAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGCCTTGCTCCGGATC | |
| | AGCTCCAAACTGTCGGGGTTTTTCTTCCGGGGCATCAGGCTG | |
| | CTTCCCGTGCTGGGGATTCCAGgctaagcAGCTTGGCGTAACTA | |
| | GATCT | |
| Argininosuccinate | ggccaaagcatgcatGGGGAAGCAGCCTGATGCCCGTTTCAGAGCT | 207 |
| lyase | ATGCTGGAAACAGCATAGCAAGTTGAAATAAGGCTAGTCCGTT | |
| deficiency; | ATCAACTTGAAAAAGTGGCACCGAGTCGGTGCGTTTTTCTTCT | |
| not provided | GGGGCATCAGGCTGCTTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTTTGCTCCGGATC | |
| | AGCTCCAAACTGTCGGGGTTTTTCTTCCGGGGCATCAGGCTG | |
| | CTTCCCGTGCTGGGGAAGTTCGgctaagcAGCTTGGCGTAACTA | |
| | GATCT | |
| Argininosuccinate | ggccaaagcatgcatGGGGAAGCAGCCTGATGCCCGTTTCAGAGCT | 208 |
| lyase | ATGCTGGAAACAGCATAGCAAGTTGAAATAAGGCTAGTCCGTT | |
| deficiency; | ATCAACTTGAAAAAGTGGCACCGAGTCGGTGCTCGGGGTTTTT | |
| not provided | CTTCTGGGGCATCAGGCTGCTTAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTCCGGATCA | |
| | GCTCCAAACTGTCGGGGTTTTTCTTCCGGGGCATCAGGCTGC | |
| | TTCCCGTGCTGGGGAGGCACAgctaagcAGCTTGGCGTAACTAG | |
| | ATCT | |
| Argininosuccinate | ggccaaagcatgcatGACTGTCGGGGTTTTTCTTCGTTTCAGAGCTA | 209 |
| lyase | TGCTGGAAACAGCATAGCAAGTTGAAATAAGGCTAGTCCGTTA | |
| deficiency; | TCAACTTGAAAAAGTGGCACCGAGTCGGTGCCCCAGAAGAAA | |
| not provided | AACCCCGACAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTTCAGCGCCAGCACCTCTGTC | |
| | CCCAGCACGGGAAGCAGCCTGATGCCCCGGAAGAAAAACCC | |
| | CGACAGTTTGGAGCTGAAAAACTgctaagcAGCTTGGCGTAACTA | |
| | GATCT | |
| Argininosuccinate | ggccaaagcatgcatGACTGTCGGGGTTTTTCTTCGTTTCAGAGCTA | 210 |
| lyase | TGCTGGAAACAGCATAGCAAGTTGAAATAAGGCTAGTCCGTTA | |
| deficiency; | TCAACTTGAAAAAGTGGCACCGAGTCGGTGCATGCCCCAGAA | |
| not provided | GAAAAACCCCGACAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTCGCCAGCACCTCTGTC | |
| | CCCAGCACGGGAAGCAGCCTGATGCCCCGGAAGAAAAACCC | |
| | CGACAGTTTGGAGCTGACTGGCTgctaagcAGCTTGGCGTAACT | |
| | AGATCT | |
| Argininosuccinate | ggccaaagcatgcatGACTGTCGGGGTTTTTCTTCGTTTCAGAGCTA | 211 |
| lyase | TGCTGGAAACAGCATAGCAAGTTGAAATAAGGCTAGTCCGTTA | |
| deficiency; | TCAACTTGAAAAAGTGGCACCGAGTCGGTGCCTGATGCCCCA | |
| not provided | GAAGAAAAACCCCGACAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTCAGCACCTCTGTC | |
| | CCCAGCACGGGAAGCAGCCTGATGCCCCGGAAGAAAAACCC | |
| | CGACAGTTTGGAGCTGATGCACGgctaagcAGCTTGGCGTAACT | |
| | AGATCT | |
| Argininosuccinate | ggccaaagcatgcatGACTGTCGGGGTTTTTCTTCGTTTCAGAGCTA | 212 |
| lyase | TGCTGGAAACAGCATAGCAAGTTGAAATAAGGCTAGTCCGTTA | |
| deficiency; | TCAACTTGAAAAAGTGGCACCGAGTCGGTGCGCAGCCTGATG | |
| not provided | CCCCAGAAGAAAAACCCCGACAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCCTCTGTCC | |
| | CCAGCACGGGAAGCAGCCTGATGCCCCGGAAGAAAAACCCC | |
| | GACAGTTTGGAGCTGAACGAGGgctaagcAGCTTGGCGTAACTA | |
| | GATCT | |
| Argininosuccinate | ggccaaagcatgcatGCTGTCGGGGTTTTTCTTCCGTTTCAGAGCTA | 213 |
| lyase | TGCTGGAAACAGCATAGCAAGTTGAAATAAGGCTAGTCCGTTA | |
| deficiency; | TCAACTTGAAAAAGTGGCACCGAGTCGGTGCCCCAGAAGAAA | |
| not provided | AACCCCGAAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTCTTCAGCGCCAGCACCTCTGT | |
| | CCCCAGCACGGGAAGCAGCCTGATGCCCCGGAAGAAAAACC | |
| | CCGACAGTTTGGAGCTGGAAATGgctaagcAGCTTGGCGTAACT | |
| | AGATCT | |
| Argininosuccinate | ggccaaagcatgcatGCTGTCGGGGTTTTTCTTCCGTTTCAGAGCTA | 214 |
| lyase | TGCTGGAAACAGCATAGCAAGTTGAAATAAGGCTAGTCCGTTA | |
| deficiency; | TCAACTTGAAAAAGTGGCACCGAGTCGGTGCATGCCCCAGAA | |
| not provided | GAAAAACCCCGAAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTAGCGCCAGCACCTCTGT | |
| | CCCCAGCACGGGAAGCAGCCTGATGCCCCGGAAGAAAAACC | |
| | CCGACAGTTTGGAGCTGCGGATTgctaagcAGCTTGGCGTAACT | |
| | AGATCT | |
| Argininosuccinate | ggccaaagcatgcatGCTGTCGGGGTTTTTCTTCCGTTTCAGAGCTA | 215 |
| lyase | TGCTGGAAACAGCATAGCAAGTTGAAATAAGGCTAGTCCGTTA | |
| deficiency; | TCAACTTGAAAAAGTGGCACCGAGTCGGTGCCTGATGCCCCA | |
| not provided | GAAGAAAAACCCCGAAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTGCCAGCACCTCTGT | |
| | CCCCAGCACGGGAAGCAGCCTGATGCCCCGGAAGAAAAACC | |
| | CCGACAGTTTGGAGCTGACTGGAgctaagcAGCTTGGCGTAACT | |
| | AGATCT | |
| Argininosuccinate | ggccaaagcatgcatGCTGTCGGGGTTTTTCTTCCGTTTCAGAGCTA | 216 |
| lyase | TGCTGGAAACAGCATAGCAAGTTGAAATAAGGCTAGTCCGTTA | |
| deficiency; | TCAACTTGAAAAAGTGGCACCGAGTCGGTGCGCAGCCTGATG | |
| not provided | CCCCAGAAGAAAAACCCCGAAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCACCTCTGT | |
| | CCCCAGCACGGGAAGCAGCCTGATGCCCCGGAAGAAAAACC | |
| | CCGACAGTTTGGAGCTGAGGGAGgctaagcAGCTTGGCGTAACT | |
| | AGATCT | |
| Phenylketonuria; | ggccaaagcatgcatGCATACCTGTAATTCACCAAGTTTCAGAGCTA | 217 |
| not | TGCTGGAAACAGCATAGCAAGTTGAAATAAGGCTAGTCCGTTA | |
| provided | TCAACTTGAAAAAGTGGCACCGAGTCGGTGCCTCCTGTCATCC | |
| | TTTGGTGAATTACAGGTAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTGACTCCATAAAGG | |
| | CATATGGTGCTGGGCTCGTGTCATCCTTTGGTGAATTACAGGT | |
| | ATGACCTTCACAGGGAGAAgctaagcAGCTTGGCGTAACTAGATC | |
| | T | |
| Phenylketonuria; | ggccaaagcatgcatGCATACCTGTAATTCACCAAGTTTCAGAGCTA | 218 |
| not | TGCTGGAAACAGCATAGCAAGTTGAAATAAGGCTAGTCCGTTA | |
| provided | TCAACTTGAAAAAGTGGCACCGAGTCGGTGCTGGGCTCCTGT | |
| | CATCCTTTGGTGAATTACAGGTAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTCCATAAAG | |
| | GCATATGGTGCTGGGCTCGTGTCATCCTTTGGTGAATTACAGG | |
| | TATGACCTTCACAGCCCAGAgctaagcAGCTTGGCGTAACTAGAT | |
| | CT | |
| Phenylketonuria; | ggccaaagcatgcatGCATACCTGTAATTCACCAAGTTTCAGAGCTA | 219 |
| not | TGCTGGAAACAGCATAGCAAGTTGAAATAAGGCTAGTCCGTTA | |
| provided | TCAACTTGAAAAAGTGGCACCGAGTCGGTGCTGCTGGGCTCC | |
| | TGTCATCCTTTGGTGAATTACAGGTAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTTAAAG | |
| | GCATATGGTGCTGGGCTCGTGTCATCCTTTGGTGAATTACAGG | |
| | TATGACCTTCACAGGAAGAAgctaagcAGCTTGGCGTAACTAGAT | |
| | CT | |
| Phenylketonuria; | ggccaaagcatgcatGCATACCTGTAATTCACCAAGTTTCAGAGCTA | 220 |
| not | TGCTGGAAACAGCATAGCAAGTTGAAATAAGGCTAGTCCGTTA | |
| provided | TCAACTTGAAAAAGTGGCACCGAGTCGGTGCTATGGTGCTGG | |
| | GCTCCTGTCATCCTTTGGTGAATTACAGGTAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | GCATATGGTGCTGGGCTCGTGTCATCCTTTGGTGAATTACAGG | |
| | TATGACCTTCACAGGTTGTCgctaagcAGCTTGGCGTAACTAGAT | |
| | CT | |
| Phenylketonuria; | ggccaaagcatgcatGCCATAAAGGCATATGGTGCGTTTCAGAGCTA | 221 |
| not | TGCTGGAAACAGCATAGCAAGTTGAAATAAGGCTAGTCCGTTA | |
| provided | TCAACTTGAAAAAGTGGCACCGAGTCGGTGCGATGACAGGAG | |
| | CCCAGCACCATATGCCTTTAAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTGTCATACCTG | |
| | TAATTCACCAAAGGATGACACGAGCCCAGCACCATATGCCTTT | |
| | ATGGAGTCTCCTTGGAATAGgctaagcAGCTTGGCGTAACTAGAT | |
| | CT | |
| Phenylketonuria; | ggccaaagcatgcatGCCATAAAGGCATATGGTGCGTTTCAGAGCTA | 222 |
| not | TGCTGGAAACAGCATAGCAAGTTGAAATAAGGCTAGTCCGTTA | |
| provided | TCAACTTGAAAAAGTGGCACCGAGTCGGTGCAAGGATGACAG | |
| | GAGCCCAGCACCATATGCCTTTAAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTATACCTG | |
| | TAATTCACCAAAGGATGACACGAGCCCAGCACCATATGCCTTT | |
| | ATGGAGTCTCCTTGCGGGCAgctaagcAGCTTGGCGTAACTAGA | |
| | TCT | |
| Phenylketonuria; | ggccaaagcatgcatGCCATAAAGGCATATGGTGCGTTTCAGAGCTA | 223 |
| not | TGCTGGAAACAGCATAGCAAGTTGAAATAAGGCTAGTCCGTTA | |
| provided | TCAACTTGAAAAAGTGGCACCGAGTCGGTGCCACCAAAGGAT | |
| | GACAGGAGCCCAGCACCATATGCCTTTAAAAAAAAAAGCTGCC | |
| | ATCAGTCGGCGTGGACTGTAGAACACTGCCAATGCCGGTCCC | |
| | AAGCCCGGATAAAAGTGGAGGGTACAGTCCACGCTTTTTTTTG | |
| | TAATTCACCAAAGGATGACACGAGCCCAGCACCATATGCCTTT | |
| | ATGGAGTCTCCTTGGTTAAGgctaagcAGCTTGGCGTAACTAGAT | |
| | CT | |
| Phenylketonuria; | ggccaaagcatgcatGCATAAAGGCATATGGTGCTGTTTCAGAGCTA | 224 |
| not | TGCTGGAAACAGCATAGCAAGTTGAAATAAGGCTAGTCCGTTA | |
| provided | TCAACTTGAAAAAGTGGCACCGAGTCGGTGCACAGGAGCCCA | |
| | GCACCATATGCCTTTAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGTGAAGGTCATACCT | |
| | GTAATTCACCAAAGGATGACACGAGCCCAGCACCATATGCCTT | |
| | TATGGAGTCTCCTTGGACCAgctaagcAGCTTGGCGTAACTAGAT | |
| | CT | |
| Phenylketonuria; | ggccaaagcatgcatGCATAAAGGCATATGGTGCTGTTTCAGAGCTA | 225 |
| not | TGCTGGAAACAGCATAGCAAGTTGAAATAAGGCTAGTCCGTTA | |
| provided | TCAACTTGAAAAAGTGGCACCGAGTCGGTGCGATGACAGGAG | |
| | CCCAGCACCATATGCCTTTAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTAGGTCATACCT | |
| | GTAATTCACCAAAGGATGACACGAGCCCAGCACCATATGCCTT | |
| | TATGGAGTCTCCTTTACCGAgctaagcAGCTTGGCGTAACTAGAT | |
| | CT | |
| Phenylketonuria; | ggccaaagcatgcatGCATAAAGGCATATGGTGCTGTTTCAGAGCTA | 226 |
| not | TGCTGGAAACAGCATAGCAAGTTGAAATAAGGCTAGTCCGTTA | |
| provided | TCAACTTGAAAAAGTGGCACCGAGTCGGTGCAAGGATGACAG | |
| | GAGCCCAGCACCATATGCCTTTAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTTCATACCT | |
| | GTAATTCACCAAAGGATGACACGAGCCCAGCACCATATGCCTT | |
| | TATGGAGTCTCCTTTACGCTgctaagcAGCTTGGCGTAACTAGAT | |
| | CT | |
| Phenylketonuria; | ggccaaagcatgcatGCATAAAGGCATATGGTGCTGTTTCAGAGCTA | 227 |
| not | TGCTGGAAACAGCATAGCAAGTTGAAATAAGGCTAGTCCGTTA | |
| provided | TCAACTTGAAAAAGTGGCACCGAGTCGGTGCCACCAAAGGAT | |
| | GACAGGAGCCCAGCACCATATGCCTTTAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTCCT | |
| | GTAATTCACCAAAGGATGACACGAGCCCAGCACCATATGCCTT | |
| | TATGGAGTCTCCTTAATATTgctaagcAGCTTGGCGTAACTAGAT | |
| | CT | |
| Phenylketonuria; | ggccaaagcatgcatGAGGAAAGCAGGCCAGCCACGTTTCAGAGCT | 228 |
| not | ATGCTGGAAACAGCATAGCAAGTTGAAATAAGGCTAGTCCGTT | |
| provided | ATCAACTTGAAAAAGTGGCACCGAGTCGGTGCCTCCGACCTG | |
| | TGGCTGGCCTGCTTTAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTTTCTTCTTTTCATCC | |
| | CAGCTTGCACTGGTTTCCGCCTCTGACCTGTGGCTGGCCTGC | |
| | TTTCCTCTCGGGATTTCAGATCgctaagcAGCTTGGCGTAACTAG | |
| | ATCT | |
| Phenylketonuria; | ggccaaagcatgcatGAGGAAAGCAGGCCAGCCACGTTTCAGAGCT | 229 |
| not | ATGCTGGAAACAGCATAGCAAGTTGAAATAAGGCTAGTCCGTT | |
| provided | ATCAACTTGAAAAAGTGGCACCGAGTCGGTGCCCGCCTCCGA | |
| | CCTGTGGCTGGCCTGCTTTAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTTCTTTTCATCCC | |
| | AGCTTGCACTGGTTTCCGCCTCTGACCTGTGGCTGGCCTGCT | |
| | TTCCTCTCGGGATTTGAACTCgctaagcAGCTTGGCGTAACTAGA | |
| | TCT | |
| Phenylketonuria; | ggccaaagcatgcatGAGGAAAGCAGGCCAGCCACGTTTCAGAGCT | 230 |
| not | ATGCTGGAAACAGCATAGCAAGTTGAAATAAGGCTAGTCCGTT | |
| provided | ATCAACTTGAAAAAGTGGCACCGAGTCGGTGCTTTCCGCCTCC | |
| | GACCTGTGGCTGGCCTGCTTTAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTTTTCATCCC | |
| | AGCTTGCACTGGTTTCCGCCTCTGACCTGTGGCTGGCCTGCT | |
| | TTCCTCTCGGGATTTTTCTGTgctaagcAGCTTGGCGTAACTAGA | |
| | TCT | |
| Phenylketonuria; | ggccaaagcatgcatGAGGAAAGCAGGCCAGCCACGTTTCAGAGCT | 231 |
| not | ATGCTGGAAACAGCATAGCAAGTTGAAATAAGGCTAGTCCGTT | |
| provided | ATCAACTTGAAAAAGTGGCACCGAGTCGGTGCACTGGTTTCC | |
| | GCCTCCGACCTGTGGCTGGCCTGCTTTAAAAAAAAAGCTGCC | |
| | ATCAGTCGGCGTGGACTGTAGAACACTGCCAATGCCGGTCCC | |
| | AAGCCCGGATAAAAGTGGAGGGTACAGTCCACGCTTTTTTTTC | |
| | CCAGCTTGCACTGGTTTCCGCCTCTGACCTGTGGCTGGCCTG | |
| | CTTTCCTCTCGGGATTTGCTCGCgctaagcAGCTTGGCGTAACTA | |
| | GATCT | |
| Phenylketonuria; | ggccaaagcatgcatGCAGGCCAGCCACAGGTCAGGTTTCAGAGCT | 232 |
| not | ATGCTGGAAACAGCATAGCAAGTTGAAATAAGGCTAGTCCGTT | |
| provided | ATCAACTTGAAAAAGTGGCACCGAGTCGGTGCCTCCGACCTG | |
| | TGGCTGGCAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTGCTTTGAGTTTTCTTTCTTCTT | |
| | TTCATCCCAGCTTGCACTGGTTTCCGCCTCTGACCTGTGGCTG | |
| | GCCTGCTTTCCTCTCTACTGGgctaagcAGCTTGGCGTAACTAGA | |
| | TCT | |
| Phenylketonuria; | ggccaaagcatgcatGCAGGCCAGCCACAGGTCAGGTTTCAGAGCT | 233 |
| not | ATGCTGGAAACAGCATAGCAAGTTGAAATAAGGCTAGTCCGTT | |
| provided | ATCAACTTGAAAAAGTGGCACCGAGTCGGTGCCCGCCTCCGA | |
| | CCTGTGGCTGGCAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTGAGTTTTCTTTCTTCTT | |
| | TTCATCCCAGCTTGCACTGGTTTCCGCCTCTGACCTGTGGCTG | |
| | GCCTGCTTTCCTCTCCTTCACgctaagcAGCTTGGCGTAACTAGA | |
| | TCT | |
| Phenylketonuria; | ggccaaagcatgcatGCAGGCCAGCCACAGGTCAGGTTTCAGAGCT | 234 |
| not | ATGCTGGAAACAGCATAGCAAGTTGAAATAAGGCTAGTCCGTT | |
| provided | ATCAACTTGAAAAAGTGGCACCGAGTCGGTGCTTTCCGCCTCC | |
| | GACCTGTGGCTGGCAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGTTTTCTTTCTTCTTT | |
| | TCATCCCAGCTTGCACTGGTTTCCGCCTCTGACCTGTGGCTG | |
| | GCCTGCTTTCCTCTCTCTTGCgctaagcAGCTTGGCGTAACTAGA | |
| | TCT | |
| Phenylketonuria; | ggccaaagcatgcatGCAGGCCAGCCACAGGTCAGGTTTCAGAGCT | 235 |
| not | ATGCTGGAAACAGCATAGCAAGTTGAAATAAGGCTAGTCCGTT | |
| provided | ATCAACTTGAAAAAGTGGCACCGAGTCGGTGCACTGGTTTCC | |
| | GCCTCCGACCTGTGGCTGGCAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCTTTCTTCTT | |
| | TTCATCCCAGCTTGCACTGGTTTCCGCCTCTGACCTGTGGCTG | |
| | GCCTGCTTTCCTCTCCAAATAgctaagcAGCTTGGCGTAACTAGA | |
| | TCT | |
| Phenylketonuria; | ggccaaagcatgcatGACGGTTCGGGGGTATACATGTTTCAGAGCTA | 236 |
| not | TGCTGGAAACAGCATAGCAAGTTGAAATAAGGCTAGTCCGTTA | |
| provided | TCAACTTGAAAAAGTGGCACCGAGTCGGTGCCATGGATCCAA | |
| | GCCCATGTATACCCCCGAACAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTCCACTGCAC | |
| | ACAGTACATCAGACATTGATCCAAGCCCATGTATACCCCCGAA | |
| | CCGTGAGTACTGTCTGTGGCgctaagcAGCTTGGCGTAACTAGA | |
| | TCT | |
| Phenylketonuria; | ggccaaagcatgcatGACGGTTCGGGGGTATACATGTTTCAGAGCTA | 237 |
| not | TGCTGGAAACAGCATAGCAAGTTGAAATAAGGCTAGTCCGTTA | |
| provided | TCAACTTGAAAAAGTGGCACCGAGTCGGTGCCAGACATGGAT | |
| | CCAAGCCCATGTATACCCCCGAACAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTCTGCAC | |
| | ACAGTACATCAGACATTGATCCAAGCCCATGTATACCCCCGAA | |
| | CCGTGAGTACTGTCACAACTgctaagcAGCTTGGCGTAACTAGAT | |
| | CT | |
| Phenylketonuria; | ggccaaagcatgcatGACGGTTCGGGGGTATACATGTTTCAGAGCTA | 238 |
| not | TGCTGGAAACAGCATAGCAAGTTGAAATAAGGCTAGTCCGTTA | |
| provided | TCAACTTGAAAAAGTGGCACCGAGTCGGTGCCATCAGACATG | |
| | GATCCAAGCCCATGTATACCCCCGAACAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTCAC | |
| | ACAGTACATCAGACATTGATCCAAGCCCATGTATACCCCCGAA | |
| | CCGTGAGTACTGTCGGTACAgctaagcAGCTTGGCGTAACTAGAT | |
| | CT | |
| Phenylketonuria; | ggccaaagcatgcatGTCGGGGGTATACATGGGCTGTTTCAGAGCT | 239 |
| not | ATGCTGGAAACAGCATAGCAAGTTGAAATAAGGCTAGTCCGTT | |
| provided | ATCAACTTGAAAAAGTGGCACCGAGTCGGTGCCATGGATCCA | |
| | AGCCCATGTATACCCCAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTTTCCGAGTCTTCCA | |
| | CTGCACACAGTACATCAGACATTGATCCAAGCCCATGTATACC | |
| | CCCGAACCGTGAGTAGATCGAgctaagcAGCTTGGCGTAACTAG | |
| | ATCT | |
| Phenylketonuria; | ggccaaagcatgcatGAGGAAAGCAGGCCAGCCACGTTTCAGAGCT | 240 |
| not | ATGCTGGAAACAGCATAGCAAGTTGAAATAAGGCTAGTCCGTT | |
| provided | ATCAACTTGAAAAAGTGGCACCGAGTCGGTGCTGGTTTCCGC | |
| | CTCCGACCTGTGGCTGGCCTGCTTTAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTCATCC | |
| | CAGCTTGCACTGGTTTCTGCCTCCGACCTGTGGCTGGCCTGC | |
| | TTTCCTCTCGGGATTTGTTGCTgctaagcAGCTTGGCGTAACTAG | |
| | ATCT | |
| Phenylketonuria; | ggccaaagcatgcatGAAGCAGGCCAGCCACAGGTGTTTCAGAGCT | 241 |
| not | ATGCTGGAAACAGCATAGCAAGTTGAAATAAGGCTAGTCCGTT | |
| provided | ATCAACTTGAAAAAGTGGCACCGAGTCGGTGCTGGTTTCCGC | |
| | CTCCGACCTGTGGCTGGCCTGAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTCTTCTTTTC | |
| | ATCCCAGCTTGCACTGGTTTCTGCCTCCGACCTGTGGCTGGC | |
| | CTGCTTTCCTCTCGGGAAATCAgctaagcAGCTTGGCGTAACTAG | |
| | ATCT | |
| Phenylketonuria; | ggccaaagcatgcatGAAGCAGGCCAGCCACAGGTGTTTCAGAGCT | 242 |
| not | ATGCTGGAAACAGCATAGCAAGTTGAAATAAGGCTAGTCCGTT | |
| provided | ATCAACTTGAAAAAGTGGCACCGAGTCGGTGCCACTGGTTTCC | |
| | GCCTCCGACCTGTGGCTGGCCTGAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTCTTTTC | |
| | ATCCCAGCTTGCACTGGTTTCTGCCTCCGACCTGTGGCTGGC | |
| | CTGCTTTCCTCTCGGGCAACCCgctaagcAGCTTGGCGTAACTA | |
| | GATCT | |
| Phenylketonuria; | ggccaaagcatgcatGAAGCAGGCCAGCCACAGGTGTTTCAGAGCT | 243 |
| not | ATGCTGGAAACAGCATAGCAAGTTGAAATAAGGCTAGTCCGTT | |
| provided | ATCAACTTGAAAAAGTGGCACCGAGTCGGTGCGCTTGCACTG | |
| | GTTTCCGCCTCCGACCTGTGGCTGGCCTGAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | CATCCCAGCTTGCACTGGTTTCTGCCTCCGACCTGTGGCTGG | |
| | CCTGCTTTCCTCTCGGGTGGACGgctaagcAGCTTGGCGTAACT | |
| | AGATCT | |
| Phenylketonuria; | ggccaaagcatgcatGTTTTCATCCCAGCTTGCACGTTTCAGAGCTA | 244 |
| not | TGCTGGAAACAGCATAGCAAGTTGAAATAAGGCTAGTCCGTTA | |
| provided | TCAACTTGAAAAAGTGGCACCGAGTCGGTGCGGTCGGAGGCG | |
| | GAAACCAGTGCAAGCTGGGATGAAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTAAAGCA | |
| | GGCCAGCCACAGGTCGGAGGCAGAAACCAGTGCAAGCTGGG | |
| | ATGAAAAGAAGAAAGAACCTCTCgctaagcAGCTTGGCGTAACTA | |
| | GATCT | |
| Phenylketonuria; | ggccaaagcatgcatGTTTTCATCCCAGCTTGCACGTTTCAGAGCTA | 245 |
| not | TGCTGGAAACAGCATAGCAAGTTGAAATAAGGCTAGTCCGTTA | |
| provided | TCAACTTGAAAAAGTGGCACCGAGTCGGTGCCCACAGGTCGG | |
| | AGGCGGAAACCAGTGCAAGCTGGGATGAAAAAAAAAAGCTGC | |
| | CATCAGTCGGCGTGGACTGTAGAACACTGCCAATGCCGGTCC | |
| | CAAGCCCGGATAAAAGTGGAGGGTACAGTCCACGCTTTTTTTA | |
| | GGCCAGCCACAGGTCGGAGGCAGAAACCAGTGCAAGCTGGG | |
| | ATGAAAAGAAGAAAGAAACCCAAgctaagcAGCTTGGCGTAACTA | |
| | GATCT | |
| Phenylketonuria; | ggccaaagcatgcatGCTGAAAAAATCTCATCCTAGTTTCAGAGCTAT | 246 |
| not | GCTGGAAACAGCATAGCAAGTTGAAATAAGGCTAGTCCGTTAT | |
| provided | CAACTTGAAAAAGTGGCACCGAGTCGGTGCCACCCTGTGAGT | |
| | CCATGGCCCGTAGGATGAGATTTTTTAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTGATG | |
| | CTGACCACCCTGTGATTCCATGGCCCGTAGGATGAGATTTTTT | |
| | CAGTGCCTCTCCTGCACCGgctaagcAGCTTGGCGTAACTAGAT | |
| | CT | |
| Phenylketonuria; | ggccaaagcatgcatGCTGAAAAAATCTCATCCTAGTTTCAGAGCTAT | 247 |
| not | GCTGGAAACAGCATAGCAAGTTGAAATAAGGCTAGTCCGTTAT | |
| provided | CAACTTGAAAAAGTGGCACCGAGTCGGTGCCTGACCACCCTG | |
| | TGAGTCCATGGCCCGTAGGATGAGATTTTTTAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TTGACCACCCTGTGATTCCATGGCCCGTAGGATGAGATTTTTT | |
| | CAGTGCCTCTCCTGCCGAAgctaagcAGCTTGGCGTAACTAGAT | |
| | CT | |
| Phenylketonuria; | ggccaaagcatgcatGTGAAAAAATCTCATCCTACGTTTCAGAGCTAT | 248 |
| not | GCTGGAAACAGCATAGCAAGTTGAAATAAGGCTAGTCCGTTAT | |
| provided | CAACTTGAAAAAGTGGCACCGAGTCGGTGCTGAGTCCATGGC | |
| | CCGTAGGATGAGATTTTTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGCGGAACTGGAT | |
| | GCTGACCACCCTGTGATTCCATGGCCCGTAGGATGAGATTTTT | |
| | TCAGTGCCTCTCCCCGCAAgctaagcAGCTTGGCGTAACTAGATC | |
| | T | |
| Phenylketonuria; | ggccaaagcatgcatGTGAAAAAATCTCATCCTACGTTTCAGAGCTAT | 249 |
| not | GCTGGAAACAGCATAGCAAGTTGAAATAAGGCTAGTCCGTTAT | |
| provided | CAACTTGAAAAAGTGGCACCGAGTCGGTGCCCTGTGAGTCCA | |
| | TGGCCCGTAGGATGAGATTTTTAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTAACTGGAT | |
| | GCTGACCACCCTGTGATTCCATGGCCCGTAGGATGAGATTTTT | |
| | TCAGTGCCTCTCCTTCTCTgctaagcAGCTTGGCGTAACTAGATC | |
| | T | |
| Phenylketonuria; | ggccaaagcatgcatGTGAAAAAATCTCATCCTACGTTTCAGAGCTAT | 250 |
| not | GCTGGAAACAGCATAGCAAGTTGAAATAAGGCTAGTCCGTTAT | |
| provided | CAACTTGAAAAAGTGGCACCGAGTCGGTGCCACCCTGTGAGT | |
| | CCATGGCCCGTAGGATGAGATTTTTAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTTGGAT | |
| | GCTGACCACCCTGTGATTCCATGGCCCGTAGGATGAGATTTTT | |
| | TCAGTGCCTCTCCTTAAAGgctaagcAGCTTGGCGTAACTAGATC | |
| | T | |
| Phenylketonuria; | ggccaaagcatgcatGTGAAAAAATCTCATCCTACGTTTCAGAGCTAT | 251 |
| not | GCTGGAAACAGCATAGCAAGTTGAAATAAGGCTAGTCCGTTAT | |
| provided | CAACTTGAAAAAGTGGCACCGAGTCGGTGCCTGACCACCCTG | |
| | TGAGTCCATGGCCCGTAGGATGAGATTTTTAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | GCTGACCACCCTGTGATTCCATGGCCCGTAGGATGAGATTTTT | |
| | TCAGTGCCTCTCCACCCACgctaagcAGCTTGGCGTAACTAGATC | |
| | T | |
| Phenylketonuria; | ggccaaagcatgcatGAATCTCATCCTACGGGCCAGTTTCAGAGCTA | 252 |
| not | TGCTGGAAACAGCATAGCAAGTTGAAATAAGGCTAGTCCGTTA | |
| provided | TCAACTTGAAAAAGTGGCACCGAGTCGGTGCTGAGTCCATGG | |
| | CCCGTAGGATGAGAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTCTCAGCTATGGAGCG | |
| | GAACTGGATGCTGACCACCCTGTGATTCCATGGCCCGTAGGA | |
| | TGAGATTTTTTCAGTGCAGTTTGgctaagcAGCTTGGCGTAACTA | |
| | GATCT | |
| Phenylketonuria; | ggccaaagcatgcatGAATCTCATCCTACGGGCCAGTTTCAGAGCTA | 253 |
| not | TGCTGGAAACAGCATAGCAAGTTGAAATAAGGCTAGTCCGTTA | |
| provided | TCAACTTGAAAAAGTGGCACCGAGTCGGTGCCCTGTGAGTCC | |
| | ATGGCCCGTAGGATGAGAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGCTATGGAGCGG | |
| | AACTGGATGCTGACCACCCTGTGATTCCATGGCCCGTAGGAT | |
| | GAGATTTTTTCAGTGCAAGTAGgctaagcAGCTTGGCGTAACTAG | |
| | ATCT | |
| Phenylketonuria; | ggccaaagcatgcatGAATCTCATCCTACGGGCCAGTTTCAGAGCTA | 254 |
| not | TGCTGGAAACAGCATAGCAAGTTGAAATAAGGCTAGTCCGTTA | |
| provided | TCAACTTGAAAAAGTGGCACCGAGTCGGTGCCACCCTGTGAG | |
| | TCCATGGCCCGTAGGATGAGAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTATGGAGCGG | |
| | AACTGGATGCTGACCACCCTGTGATTCCATGGCCCGTAGGAT | |
| | GAGATTTTTTCAGTGCTCAAGAgctaagcAGCTTGGCGTAACTAG | |
| | ATCT | |
| Phenylketonuria; | ggccaaagcatgcatGAATCTCATCCTACGGGCCAGTTTCAGAGCTA | 255 |
| not | TGCTGGAAACAGCATAGCAAGTTGAAATAAGGCTAGTCCGTTA | |
| provided | TCAACTTGAAAAAGTGGCACCGAGTCGGTGCCTGACCACCCT | |
| | GTGAGTCCATGGCCCGTAGGATGAGAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTGCG | |
| | GAACTGGATGCTGACCACCCTGTGATTCCATGGCCCGTAGGA | |
| | TGAGATTTTTTCAGTGCACTCGCgctaagcAGCTTGGCGTAACTA | |
| | GATCT | |
| Phenylketonuria; | ggccaaagcatgcatGTCATACCTGTAATTCACAAGTTTCAGAGCTAT | 256 |
| not | GCTGGAAACAGCATAGCAAGTTGAAATAAGGCTAGTCCGTTAT | |
| provided | CAACTTGAAAAAGTGGCACCGAGTCGGTGCTTGGTGAATTACA | |
| | GGTAAAAAAAAAAGCTGCCATCAGTCGGCGTGGACTGTAGAA | |
| | CACTGCCAATGCCGGTCCCAAGCCCGGATAAAAGTGGAGGGT | |
| | ACAGTCCACGCTTTTTTTTGCAAACAAGGAGACTCCATAAAGG | |
| | CATATGGTGCTGGGCTCCTGTCATCCTTTGTGAATTACAGGTA | |
| | TGACCTTCACAGGCCGCCTgctaagcAGCTTGGCGTAACTAGAT | |
| | CT | |
| Phenylketonuria; | ggccaaagcatgcatGTCATACCTGTAATTCACAAGTTTCAGAGCTAT | 257 |
| not | GCTGGAAACAGCATAGCAAGTTGAAATAAGGCTAGTCCGTTAT | |
| provided | CAACTTGAAAAAGTGGCACCGAGTCGGTGCTCCTTTGGTGAAT | |
| | TACAGGTAAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTAACAAGGAGACTCCATAAAGGC | |
| | ATATGGTGCTGGGCTCCTGTCATCCTTTGTGAATTACAGGTAT | |
| | GACCTTCACAGGGATTAAgctaagcAGCTTGGCGTAACTAGATCT | |
| Phenylketonuria; | ggccaaagcatgcatGTCATACCTGTAATTCACAAGTTTCAGAGCTAT | 258 |
| not | GCTGGAAACAGCATAGCAAGTTGAAATAAGGCTAGTCCGTTAT | |
| provided | CAACTTGAAAAAGTGGCACCGAGTCGGTGCTCCTGTCATCCTT | |
| | TGGTGAATTACAGGTAAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTGACTCCATAAAGGC | |
| | ATATGGTGCTGGGCTCCTGTCATCCTTTGTGAATTACAGGTAT | |
| | GACCTTCACAGGGAGCTGgctaagcAGCTTGGCGTAACTAGATC | |
| | T | |
| Phenylketonuria; | ggccaaagcatgcatGGGCTGTCTTCTCCAGCTCCGTTTCAGAGCTA | 259 |
| not | TGCTGGAAACAGCATAGCAAGTTGAAATAAGGCTAGTCCGTTA | |
| provided | TCAACTTGAAAAAGTGGCACCGAGTCGGTGCAAAGCTTCTCCC | |
| | CCTGGAGCTGGAGAAGACAAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTACAGTACTGC | |
| | TTATCAGAGAAGCCAAACTTCTCCCCCTGGAGCTGGAGAAGA | |
| | CAGCCATCCAAAATTGCATGTgctaagcAGCTTGGCGTAACTAGA | |
| | TCT | |
| Phenylketonuria; | ggccaaagcatgcatGGGCTGTCTTCTCCAGCTCCGTTTCAGAGCTA | 260 |
| not | TGCTGGAAACAGCATAGCAAGTTGAAATAAGGCTAGTCCGTTA | |
| provided | TCAACTTGAAAAAGTGGCACCGAGTCGGTGCAGAAGCCAAAG | |
| | CTTCTCCCCCTGGAGCTGGAGAAGACAAAAAAAAAAGCTGCC | |
| | ATCAGTCGGCGTGGACTGTAGAACACTGCCAATGCCGGTCCC | |
| | AAGCCCGGATAAAAGTGGAGGGTACAGTCCACGCTTTTTTTTG | |
| | CTTATCAGAGAAGCCAAACTTCTCCCCCTGGAGCTGGAGAAG | |
| | ACAGCCATCCAAAATTAAGTCTgctaagcAGCTTGGCGTAACTAG | |
| | ATCT | |
| Phenylketonuria; | ggccaaagcatgcatGGGCTGTCTTCTCCAGCTCCGTTTCAGAGCTA | 261 |
| not | TGCTGGAAACAGCATAGCAAGTTGAAATAAGGCTAGTCCGTTA | |
| provided | TCAACTTGAAAAAGTGGCACCGAGTCGGTGCATCAGAGAAGC | |
| | CAAAGCTTCTCCCCCTGGAGCTGGAGAAGACAAAAAAAAAAG | |
| | CTGCCATCAGTCGGCGTGGACTGTAGAACACTGCCAATGCCG | |
| | GTCCCAAGCCCGGATAAAAGTGGAGGGTACAGTCCACGCTTT | |
| | TTTTATCAGAGAAGCCAAACTTCTCCCCCTGGAGCTGGAGAAG | |
| | ACAGCCATCCAAAATTCGACGCgctaagcAGCTTGGCGTAACTAG | |
| | ATCT | |
| Phenylketonuria; | ggccaaagcatgcatGCTGTCTTCTCCAGCTCCAGGTTTCAGAGCTA | 262 |
| not | TGCTGGAAACAGCATAGCAAGTTGAAATAAGGCTAGTCCGTTA | |
| provided | TCAACTTGAAAAAGTGGCACCGAGTCGGTGCAAAGCTTCTCCC | |
| | CCTGGAGCTGGAGAAGAAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGCCTACAGTACT | |
| | GCTTATCAGAGAAGCCAAACTTCTCCCCCTGGAGCTGGAGAA | |
| | GACAGCCATCCAAAAGTGCCGgctaagcAGCTTGGCGTAACTAG | |
| | ATCT | |
| Phenylketonuria; | ggccaaagcatgcatGCTGTCTTCTCCAGCTCCAGGTTTCAGAGCTA | 263 |
| not | TGCTGGAAACAGCATAGCAAGTTGAAATAAGGCTAGTCCGTTA | |
| provided | TCAACTTGAAAAAGTGGCACCGAGTCGGTGCAGAAGCCAAAG | |
| | CTTCTCCCCCTGGAGCTGGAGAAGAAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTGTACT | |
| | GCTTATCAGAGAAGCCAAACTTCTCCCCCTGGAGCTGGAGAA | |
| | GACAGCCATCCAAAAGCGGGTgctaagcAGCTTGGCGTAACTAG | |
| | ATCT | |
| Phenylketonuria; | ggccaaagcatgcatGCTGTCTTCTCCAGCTCCAGGTTTCAGAGCTA | 264 |
| not | TGCTGGAAACAGCATAGCAAGTTGAAATAAGGCTAGTCCGTTA | |
| provided | TCAACTTGAAAAAGTGGCACCGAGTCGGTGCATCAGAGAAGC | |
| | CAAAGCTTCTCCCCCTGGAGCTGGAGAAGAAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TGCTTATCAGAGAAGCCAAACTTCTCCCCCTGGAGCTGGAGAA | |
| | GACAGCCATCCAAAATCTCTTgctaagcAGCTTGGCGTAACTAGA | |
| | TCT | |
| Phenylketonuria; | ggccaaagcatgcatGAGCTCCAGGGGGAGAAGTTGTTTCAGAGCT | 265 |
| not | ATGCTGGAAACAGCATAGCAAGTTGAAATAAGGCTAGTCCGTT | |
| provided | ATCAACTTGAAAAAGTGGCACCGAGTCGGTGCAAAGCTTCTCC | |
| | CCCTGGAAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTTGATAATAACTTTTCACTTGGGG | |
| | CCTACAGTACTGCTTATCAGAGAAGCCAAACTTCTCCCCCTGG | |
| | AGCTGGAGAAGACAAATTCGgctaagcAGCTTGGCGTAACTAGAT | |
| | CT | |
| Phenylketonuria; | ggccaaagcatgcatGAGCTCCAGGGGGAGAAGTTGTTTCAGAGCT | 266 |
| not | ATGCTGGAAACAGCATAGCAAGTTGAAATAAGGCTAGTCCGTT | |
| provided | ATCAACTTGAAAAAGTGGCACCGAGTCGGTGCAGCCAAAGCT | |
| | TCTCCCCCTGGAAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTAATAACTTTTCACTTGGG | |
| | GCCTACAGTACTGCTTATCAGAGAAGCCAAACTTCTCCCCCTG | |
| | GAGCTGGAGAAGACAAGTAACgctaagcAGCTTGGCGTAACTAG | |
| | ATCT | |
| Phenylketonuria; | ggccaaagcatgcatGAGCTCCAGGGGGAGAAGTTGTTTCAGAGCT | 267 |
| not | ATGCTGGAAACAGCATAGCAAGTTGAAATAAGGCTAGTCCGTT | |
| provided | ATCAACTTGAAAAAGTGGCACCGAGTCGGTGCAGAAGCCAAA | |
| | GCTTCTCCCCCTGGAAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTAACTTTTCACTTGG | |
| | GGCCTACAGTACTGCTTATCAGAGAAGCCAAACTTCTCCCCCT | |
| | GGAGCTGGAGAAGACAACATGCgctaagcAGCTTGGCGTAACTA | |
| | GATCT | |
| Phenylketonuria; | ggccaaagcatgcatGAGCTCCAGGGGGAGAAGTTGTTTCAGAGCT | 268 |
| not | ATGCTGGAAACAGCATAGCAAGTTGAAATAAGGCTAGTCCGTT | |
| provided | ATCAACTTGAAAAAGTGGCACCGAGTCGGTGCATCAGAGAAG | |
| | CCAAAGCTTCTCCCCCTGGAAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTTCACTTGGG | |
| | GCCTACAGTACTGCTTATCAGAGAAGCCAAACTTCTCCCCCTG | |
| | GAGCTGGAGAAGACAGTCCAGgctaagcAGCTTGGCGTAACTAG | |
| | ATCT | |
| Ornithine | ggccaaagcatgcatGAAAATAAAGTGCAGCTGAAGTTTCAGAGCTA | 269 |
| carbamoyl- | TGCTGGAAACAGCATAGCAAGTTGAAATAAGGCTAGTCCGTTA | |
| transferase | TCAACTTGAAAAAGTGGCACCGAGTCGGTGCTCACGGCCCTT | |
| deficiency; | CAGCTGCACTTTATAAAAAAAAAGCTGCCATCAGTCGGCGTGG | |
| not provided | ACTGTAGAACACTGCCAATGCCGGTCCCAAGCCCGGATAAAA | |
| | GTGGAGGGTACAGTCCACGCTTTTTTTTTTCTTCTCCGGTAAA | |
| | GTTTTTTAGAGTGAGAAGGTCATGGCCCTTCAGCTGCACTTTA | |
| | TTTTGTAGTGGTTGAGAATGgctaagcAGCTTGGCGTAACTAGAT | |
| | CT | |
| Ornithine | ggccaaagcatgcatGAAAATAAAGTGCAGCTGAAGTTTCAGAGCTA | 270 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAAGGTCACGGC | |
| deficiency; | CCTTCAGCTGCACTTTATAAAAAAAAAGCTGCCATCAGTCGGC | |
| not provided | GTGGACTGTAGAACACTGCCAATGCCGGTCCCAAGCCCGGAT | |
| | AAAAGTGGAGGGTACAGTCCACGCTTTTTTTTTCTCCGGTAAA | |
| | GTTTTTTAGAGTGAGAAGGTCATGGCCCTTCAGCTGCACTTTA | |
| | TTTTGTAGTGGTTGCTTATAgctaagcAGCTTGGCGTAACTAGAT | |
| | CT | |
| Ornithine | ggccaaagcatgcatGAAAATAAAGTGCAGCTGAAGTTTCAGAGCTA | 271 |
| carbamoyl- | TGCTGGAAACAGCATAGCAAGTTGAAATAAGGCTAGTCCGTTA | |
| transferase | TCAACTTGAAAAAGTGGCACCGAGTCGGTGCGAGAAGGTCAC | |
| deficiency; | GGCCCTTCAGCTGCACTTTATAAAAAAAAAGCTGCCATCAGTC | |
| not provided | GGCGTGGACTGTAGAACACTGCCAATGCCGGTCCCAAGCCCG | |
| | GATAAAAGTGGAGGGTACAGTCCACGCTTTTTTTTCCGGTAAA | |
| | GTTTTTTAGAGTGAGAAGGTCATGGCCCTTCAGCTGCACTTTA | |
| | TTTTGTAGTGGTTGGTTAATgctaagcAGCTTGGCGTAACTAGAT | |
| | CT | |
| Ornithine | ggccaaagcatgcatGAAAATAAAGTGCAGCTGAAGTTTCAGAGCTA | 272 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAGAGTGAGAAG | |
| deficiency; | GTCACGGCCCTTCAGCTGCACTTTATAAAAAAAAAGCTGCCAT | |
| not provided | CAGTCGGCGTGGACTGTAGAACACTGCCAATGCCGGTCCCAA | |
| | GCCCGGATAAAAGTGGAGGGTACAGTCCACGCTTTTTTTTAAA | |
| | GTTTTTTAGAGTGAGAAGGTCATGGCCCTTCAGCTGCACTTTA | |
| | TTTTGTAGTGGTTGATACTGgctaagcAGCTTGGCGTAACTAGAT | |
| | CT | |
| Ornithine | ggccaaagcatgcatGAAAGTTTTTTAGAGTGAGAGTTTCAGAGCTA | 273 |
| carbamoyl- | TGCTGGAAACAGCATAGCAAGTTGAAATAAGGCTAGTCCGTTA | |
| transferase | TCAACTTGAAAAAGTGGCACCGAGTCGGTGCGCCGTGACCTT | |
| deficiency; | CTCACTCTAAAAAACAAAAAAAAAGCTGCCATCAGTCGGCGTG | |
| not provided | GACTGTAGAACACTGCCAATGCCGGTCCCAAGCCCGGATAAA | |
| | AGTGGAGGGTACAGTCCACGCTTTTTTTGACAACCACTACAAA | |
| | ATAAAGTGCAGCTGAAGGGCCATGACCTTCTCACTCTAAAAAA | |
| | CTTTACCGGAGAAGGAGGCAgctaagcAGCTTGGCGTAACTAGA | |
| | TCT | |
| Ornithine | ggccaaagcatgcatGAAAGTTTTTTAGAGTGAGAGTTTCAGAGCTA | 274 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAAGGGCCGTGA | |
| deficiency; | CCTTCTCACTCTAAAAAACAAAAAAAAAGCTGCCATCAGTCGG | |
| not provided | CGTGGACTGTAGAACACTGCCAATGCCGGTCCCAAGCCCGGA | |
| | TAAAAGTGGAGGGTACAGTCCACGCTTTTTTTACCACTACAAA | |
| | ATAAAGTGCAGCTGAAGGGCCATGACCTTCTCACTCTAAAAAA | |
| | CTTTACCGGAGAAGCTTACAgctaagcAGCTTGGCGTAACTAGAT | |
| | CT | |
| Ornithine | ggccaaagcatgcatGAAAGTTTTTTAGAGTGAGAGTTTCAGAGCTA | 275 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCTGAAGGGCCG | |
| deficiency; | TGACCTTCTCACTCTAAAAAACAAAAAAAAAGCTGCCATCAGTC | |
| not provided | GGCGTGGACTGTAGAACACTGCCAATGCCGGTCCCAAGCCCG | |
| | GATAAAAGTGGAGGGTACAGTCCACGCTTTTTTTACTACAAAA | |
| | TAAAGTGCAGCTGAAGGGCCATGACCTTCTCACTCTAAAAAAC | |
| | TTTACCGGAGAAGTTTAACgctaagcAGCTTGGCGTAACTAGATC | |
| | T | |
| Ornithine | ggccaaagcatgcatGAAAGTTTTTTAGAGTGAGAGTTTCAGAGCTA | 276 |
| carbamoyl- | TGCTGGAAACAGCATAGCAAGTTGAAATAAGGCTAGTCCGTTA | |
| transferase | TCAACTTGAAAAAGTGGCACCGAGTCGGTGCTGCAGCTGAAG | |
| deficiency; | GGCCGTGACCTTCTCACTCTAAAAAACAAAAAAAAAGCTGCCA | |
| not provided | TCAGTCGGCGTGGACTGTAGAACACTGCCAATGCCGGTCCCA | |
| | AGCCCGGATAAAAGTGGAGGGTACAGTCCACGCTTTTTTTAAA | |
| | ATAAAGTGCAGCTGAAGGGCCATGACCTTCTCACTCTAAAAAA | |
| | CTTTACCGGAGAAGTGTGTTgctaagcAGCTTGGCGTAACTAGAT | |
| | CT | |
| Ornithine | ggccaaagcatgcatGTTTTAGAGTGAGAAGGTCAGTTTCAGAGCTA | 277 |
| carbamoyl- | TGCTGGAAACAGCATAGCAAGTTGAAATAAGGCTAGTCCGTTA | |
| transferase | TCAACTTGAAAAAGTGGCACCGAGTCGGTGCGCCGTGACCTT | |
| deficiency; | CTCACTCTAAAAAAAAAAGCTGCCATCAGTCGGCGTGGACTGT | |
| not provided | AGAACACTGCCAATGCCGGTCCCAAGCCCGGATAAAAGTGGA | |
| | GGGTACAGTCCACGCTTTTTTTTTTACAGGTGTGGACAACCAC | |
| | TACAAAATAAAGTGCAGCTGAAGGGCCATGACCTTCTCACTCT | |
| | AAAAAACTTTACCGCAGAATgctaagcAGCTTGGCGTAACTAGAT | |
| | CT | |
| Ornithine | ggccaaagcatgcatGTTTTAGAGTGAGAAGGTCAGTTTCAGAGCTA | 278 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAAGGGCCGTGA | |
| deficiency; | CCTTCTCACTCTAAAAAAAAAAGCTGCCATCAGTCGGCGTGGA | |
| not provided | CTGTAGAACACTGCCAATGCCGGTCCCAAGCCCGGATAAAAG | |
| | TGGAGGGTACAGTCCACGCTTTTTTTCAGGTGTGGACAACCAC | |
| | TACAAAATAAAGTGCAGCTGAAGGGCCATGACCTTCTCACTCT | |
| | AAAAAACTTTACCGACGACAgctaagcAGCTTGGCGTAACTAGAT | |
| | CT | |
| Ornithine | ggccaaagcatgcatGTTTTAGAGTGAGAAGGTCAGTTTCAGAGCTA | 279 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCTGAAGGGCCG | |
| deficiency; | TGACCTTCTCACTCTAAAAAAAAAAGCTGCCATCAGTCGGCGT | |
| not provided | GGACTGTAGAACACTGCCAATGCCGGTCCCAAGCCCGGATAA | |
| | AAGTGGAGGGTACAGTCCACGCTTTTTTTGTGTGGACAACCAC | |
| | TACAAAATAAAGTGCAGCTGAAGGGCCATGACCTTCTCACTCT | |
| | AAAAAACTTTACCGCCCTATgctaagcAGCTTGGCGTAACTAGAT | |
| | CT | |
| Ornithine | ggccaaagcatgcatGTTTTAGAGTGAGAAGGTCAGTTTCAGAGCTA | 280 |
| carbamoyl- | TGCTGGAAACAGCATAGCAAGTTGAAATAAGGCTAGTCCGTTA | |
| transferase | TCAACTTGAAAAAGTGGCACCGAGTCGGTGCTGCAGCTGAAG | |
| deficiency; | GGCCGTGACCTTCTCACTCTAAAAAAAAAAGCTGCCATCAGTC | |
| not provided | GGCGTGGACTGTAGAACACTGCCAATGCCGGTCCCAAGCCCG | |
| | GATAAAAGTGGAGGGTACAGTCCACGCTTTTTTTGACAACCAC | |
| | TACAAAATAAAGTGCAGCTGAAGGGCCATGACCTTCTCACTCT | |
| | AAAAAACTTTACCGTACCACgctaagcAGCTTGGCGTAACTAGAT | |
| | CT | |
| Phenylketonuria; | ggccaaagcatgcatGCCTCAATCCTTTGGGTGTAGTTTCAGAGCTA | 281 |
| not | TGCTGGAAACAGCATAGCAAGTTGAAATAAGGCTAGTCCGTTA | |
| provided | TCAACTTGAAAAAGTGGCACCGAGTCGGTGCTTCGCTACGAC | |
| | CCATACACCCAAAGGATTGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTGCCACAATACC | |
| | TCGGCCCTTCTCAGTTCCCTACGACCCATACACCCAAAGGATT | |
| | GAGGTCTTGGACAAGTGCACgctaagcAGCTTGGCGTAACTAGA | |
| | TCT | |
| Phenylketonuria; | ggccaaagcatgcatGCCTCAATCCTTTGGGTGTAGTTTCAGAGCTA | 282 |
| not | TGCTGGAAACAGCATAGCAAGTTGAAATAAGGCTAGTCCGTTA | |
| provided | TCAACTTGAAAAAGTGGCACCGAGTCGGTGCGGCCCTTCTCA | |
| | GTTCGCTACGACCCATACACCCAAAGGATTGAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TCGGCCCTTCTCAGTTCCCTACGACCCATACACCCAAAGGATT | |
| | GAGGTCTTGGACAAGTTAGAgctaagcAGCTTGGCGTAACTAGAT | |
| | CT | |
| Phenylketonuria; | ggccaaagcatgcatGCTTTGGGTGTATGGGTCGTGTTTCAGAGCTA | 283 |
| not | TGCTGGAAACAGCATAGCAAGTTGAAATAAGGCTAGTCCGTTA | |
| provided | TCAACTTGAAAAAGTGGCACCGAGTCGGTGCTTCGCTACGAC | |
| | CCATACACCCAAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTTCTTAGGAACTTTGCTGCC | |
| | ACAATACCTCGGCCCTTCTCAGTTCCCTACGACCCATACACCC | |
| | AAAGGATTGAGGTCGTGATCgctaagcAGCTTGGCGTAACTAGAT | |
| | CT | |
| Phenylketonuria; | ggccaaagcatgcatGCTTTGGGTGTATGGGTCGTGTTTCAGAGCTA | 284 |
| not | TGCTGGAAACAGCATAGCAAGTTGAAATAAGGCTAGTCCGTTA | |
| provided | TCAACTTGAAAAAGTGGCACCGAGTCGGTGCTCAGTTCGCTAC | |
| | GACCCATACACCCAAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTAGGAACTTTGCTGCC | |
| | ACAATACCTCGGCCCTTCTCAGTTCCCTACGACCCATACACCC | |
| | AAAGGATTGAGGTCCGATAGgctaagcAGCTTGGCGTAACTAGAT | |
| | CT | |
| Phenylketonuria; | ggccaaagcatgcatGCTTTGGGTGTATGGGTCGTGTTTCAGAGCTA | 285 |
| not | TGCTGGAAACAGCATAGCAAGTTGAAATAAGGCTAGTCCGTTA | |
| provided | TCAACTTGAAAAAGTGGCACCGAGTCGGTGCTTCTCAGTTCGC | |
| | TACGACCCATACACCCAAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTAACTTTGCTGCCA | |
| | CAATACCTCGGCCCTTCTCAGTTCCCTACGACCCATACACCCA | |
| | AAGGATTGAGGTCACGGCGgctaagcAGCTTGGCGTAACTAGAT | |
| | CT | |
| Phenylketonuria; | ggccaaagcatgcatGCTTTGGGTGTATGGGTCGTGTTTCAGAGCTA | 286 |
| not | TGCTGGAAACAGCATAGCAAGTTGAAATAAGGCTAGTCCGTTA | |
| provided | TCAACTTGAAAAAGTGGCACCGAGTCGGTGCGGCCCTTCTCA | |
| | GTTCGCTACGACCCATACACCCAAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTTGCTGCC | |
| | ACAATACCTCGGCCCTTCTCAGTTCCCTACGACCCATACACCC | |
| | AAAGGATTGAGGTCTCCGAAgctaagcAGCTTGGCGTAACTAGAT | |
| | CT | |
| Phenylketonuria; | ggccaaagcatgcatGTTTGGGTGTATGGGTCGTAGTTTCAGAGCTA | 287 |
| not | TGCTGGAAACAGCATAGCAAGTTGAAATAAGGCTAGTCCGTTA | |
| provided | TCAACTTGAAAAAGTGGCACCGAGTCGGTGCTTCGCTACGAC | |
| | CCATACACCCAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTGGTCTTAGGAACTTTGCTG | |
| | CCACAATACCTCGGCCCTTCTCAGTTCCCTACGACCCATACAC | |
| | CCAAAGGATTGAGGTCGGGGCgctaagcAGCTTGGCGTAACTAG | |
| | ATCT | |
| Phenylketonuria; | ggccaaagcatgcatGTTTGGGTGTATGGGTCGTAGTTTCAGAGCTA | 288 |
| not | TGCTGGAAACAGCATAGCAAGTTGAAATAAGGCTAGTCCGTTA | |
| provided | TCAACTTGAAAAAGTGGCACCGAGTCGGTGCTCAGTTCGCTAC | |
| | GACCCATACACCCAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTTTAGGAACTTTGCTGC | |
| | CACAATACCTCGGCCCTTCTCAGTTCCCTACGACCCATACACC | |
| | CAAAGGATTGAGGTATATGAgctaagcAGCTTGGCGTAACTAGAT | |
| | CT | |
| Phenylketonuria; | ggccaaagcatgcatGTTTGGGTGTATGGGTCGTAGTTTCAGAGCTA | 289 |
| not | TGCTGGAAACAGCATAGCAAGTTGAAATAAGGCTAGTCCGTTA | |
| provided | TCAACTTGAAAAAGTGGCACCGAGTCGGTGCTTCTCAGTTCGC | |
| | TACGACCCATACACCCAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTGGAACTTTGCTGCC | |
| | ACAATACCTCGGCCCTTCTCAGTTCCCTACGACCCATACACCC | |
| | AAAGGATTGAGGTTAAAGCgctaagcAGCTTGGCGTAACTAGATC | |
| | T | |
| Phenylketonuria; | ggccaaagcatgcatGTTTGGGTGTATGGGTCGTAGTTTCAGAGCTA | 290 |
| not | TGCTGGAAACAGCATAGCAAGTTGAAATAAGGCTAGTCCGTTA | |
| provided | TCAACTTGAAAAAGTGGCACCGAGTCGGTGCGGCCCTTCTCA | |
| | GTTCGCTACGACCCATACACCCAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTTTTGCTGC | |
| | CACAATACCTCGGCCCTTCTCAGTTCCCTACGACCCATACACC | |
| | CAAAGGATTGAGGTTTGCCTgctaagcAGCTTGGCGTAACTAGAT | |
| | CT | |
| Citrin | ggccaaagcatgcatGATCCGTTCAATGTCTGCTAGTTTCAGAGCTA | 291 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCACGTATGACCTT | |
| type | AGCAGACATTGAACGAAAAAAAAAGCTGCCATCAGTCGGCGT | |
| II; Neonatal | GGACTGTAGAACACTGCCAATGCCGGTCCCAAGCCCGGATAA | |
| intrahepatic | AAGTGGAGGGTACAGTCCACGCTTTTTTTTATATTTGTTGCTTG | |
| cholestasis | TGTTTGTTTTTCCCCTACAGACGACCTTAGCAGACATTGAACG | |
| caused by | GATTGCTCCTCTGGTGATTgctaagcAGCTTGGCGTAACTAGATC | |
| citrin | T | |
| deficiency; | | |
| not provided | | |
| Citrin | ggccaaagcatgcatGATCCGTTCAATGTCTGCTAGTTTCAGAGCTA | 292 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCACAGACGTATG | |
| type | ACCTTAGCAGACATTGAACGAAAAAAAAAGCTGCCATCAGTCG | |
| II; Neonatal | GCGTGGACTGTAGAACACTGCCAATGCCGGTCCCAAGCCCGG | |
| intrahepatic | ATAAAAGTGGAGGGTACAGTCCACGCTTTTTTTTTTGTTGCTTG | |
| cholestasis | TGTTTGTTTTTCCCCTACAGACGACCTTAGCAGACATTGAACG | |
| caused by | GATTGCTCCTCTGGTCGACgctaagcAGCTTGGCGTAACTAGATC | |
| citrin | T | |
| deficiency; | | |
| not provided | | |
| Citrin | ggccaaagcatgcatGATCCGTTCAATGTCTGCTAGTTTCAGAGCTA | 293 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCCCTACAGACGT | |
| type | ATGACCTTAGCAGACATTGAACGAAAAAAAAAGCTGCCATCAG | |
| II; Neonatal | TCGGCGTGGACTGTAGAACACTGCCAATGCCGGTCCCAAGCC | |
| intrahepatic | CGGATAAAAGTGGAGGGTACAGTCCACGCTTTTTTTGTTGCTT | |
| cholestasis | GTGTTTGTTTTTCCCCTACAGACGACCTTAGCAGACATTGAAC | |
| caused by | GGATTGCTCCTCTGTCGACTgctaagcAGCTTGGCGTAACTAGAT | |
| citrin | CT | |
| deficiency; | | |
| not provided | | |
| Citrin | ggccaaagcatgcatGATCCGTTCAATGTCTGCTAGTTTCAGAGCTA | 294 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCTTTCCCCTACAG | |
| type | ACGTATGACCTTAGCAGACATTGAACGAAAAAAAAAGCTGCCA | |
| II; Neonatal | TCAGTCGGCGTGGACTGTAGAACACTGCCAATGCCGGTCCCA | |
| intrahepatic | AGCCCGGATAAAAGTGGAGGGTACAGTCCACGCTTTTTTTTTG | |
| cholestasis | TGTTTGTTTTTCCCCTACAGACGACCTTAGCAGACATTGAACG | |
| caused by | GATTGCTCCTCTGAATTGTgctaagcAGCTTGGCGTAACTAGATC | |
| citrin | T | |
| deficiency; | | |
| not provided | | |
| Phenylketonuria; | ggccaaagcatgcatGGGGTCGTAGCGAACTGAGAGTTTCAGAGCT | 295 |
| not | ATGCTGGAAACAGCATAGCAAGTTGAAATAAGGCTAGTCCGTT | |
| provided | ATCAACTTGAAAAAGTGGCACCGAGTCGGTGCCTCGGCCCTT | |
| | CTCAGTTCGCTACGAAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTGGTTTTGGTCTTAG | |
| | GAACTTTGCTGCCACAATACCTCAGCCCTTCTCAGTTCGCTAC | |
| | GACCCATACACCCAAGAAAGAgctaagcAGCTTGGCGTAACTAG | |
| | ATCT | |
| Phenylketonuria; | ggccaaagcatgcatGGGGTCGTAGCGAACTGAGAGTTTCAGAGCT | 296 |
| not | ATGCTGGAAACAGCATAGCAAGTTGAAATAAGGCTAGTCCGTT | |
| provided | ATCAACTTGAAAAAGTGGCACCGAGTCGGTGCATACCTCGGC | |
| | CCTTCTCAGTTCGCTACGAAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTTTTGGTCTTAG | |
| | GAACTTTGCTGCCACAATACCTCAGCCCTTCTCAGTTCGCTAC | |
| | GACCCATACACCCAAAATACTgctaagcAGCTTGGCGTAACTAGA | |
| | TCT | |
| Phenylketonuria; | ggccaaagcatgcatGGGGTCGTAGCGAACTGAGAGTTTCAGAGCT | 297 |
| not | ATGCTGGAAACAGCATAGCAAGTTGAAATAAGGCTAGTCCGTT | |
| provided | ATCAACTTGAAAAAGTGGCACCGAGTCGGTGCACAATACCTCG | |
| | GCCCTTCTCAGTTCGCTACGAAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTGGTCTTAGG | |
| | AACTTTGCTGCCACAATACCTCAGCCCTTCTCAGTTCGCTACG | |
| | ACCCATACACCCAACCATCCgctaagcAGCTTGGCGTAACTAGAT | |
| | CT | |
| Phenylketonuria; | ggccaaagcatgcatGGGGTCGTAGCGAACTGAGAGTTTCAGAGCT | 298 |
| not | ATGCTGGAAACAGCATAGCAAGTTGAAATAAGGCTAGTCCGTT | |
| provided | ATCAACTTGAAAAAGTGGCACCGAGTCGGTGCCTGCCACAATA | |
| | CCTCGGCCCTTCTCAGTTCGCTACGAAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTTAGG | |
| | AACTTTGCTGCCACAATACCTCAGCCCTTCTCAGTTCGCTACG | |
| | ACCCATACACCCAAACGTCAgctaagcAGCTTGGCGTAACTAGAT | |
| | CT | |
| Phenylketonuria; | ggccaaagcatgcatGGGTCGTAGCGAACTGAGAAGTTTCAGAGCT | 299 |
| not | ATGCTGGAAACAGCATAGCAAGTTGAAATAAGGCTAGTCCGTT | |
| provided | ATCAACTTGAAAAAGTGGCACCGAGTCGGTGCATACCTCGGC | |
| | CCTTCTCAGTTCGCTACGAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGTTTTGGTCTTA | |
| | GGAACTTTGCTGCCACAATACCTCAGCCCTTCTCAGTTCGCTA | |
| | CGACCCATACACCCATGAGAAgctaagcAGCTTGGCGTAACTAG | |
| | ATCT | |
| Phenylketonuria; | ggccaaagcatgcatGGGTCGTAGCGAACTGAGAAGTTTCAGAGCT | 300 |
| not | ATGCTGGAAACAGCATAGCAAGTTGAAATAAGGCTAGTCCGTT | |
| provided | ATCAACTTGAAAAAGTGGCACCGAGTCGGTGCACAATACCTCG | |
| | GCCCTTCTCAGTTCGCTACGAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTTGGTCTTAG | |
| | GAACTTTGCTGCCACAATACCTCAGCCCTTCTCAGTTCGCTAC | |
| | GACCCATACACCCAAGTAGCgctaagcAGCTTGGCGTAACTAGAT | |
| | CT | |
| Phenylketonuria; | ggccaaagcatgcatGAGCGAACTGAGAAGGGCTGGTTTCAGAGCT | 301 |
| not | ATGCTGGAAACAGCATAGCAAGTTGAAATAAGGCTAGTCCGTT | |
| provided | ATCAACTTGAAAAAGTGGCACCGAGTCGGTGCATACCTCGGC | |
| | CCTTCTCAGTTCAAAAAAAAAGCTGCCATCAGTCGGCGTGGAC | |
| | TGTAGAACACTGCCAATGCCGGTCCCAAGCCCGGATAAAAGT | |
| | GGAGGGTACAGTCCACGCTTTTTTTCTCAAGCCTGTGGTTTTG | |
| | GTCTTAGGAACTTTGCTGCCACAATACCTCAGCCCTTCTCAGT | |
| | TCGCTACGACCCATACCGACTgctaagcAGCTTGGCGTAACTAGA | |
| | TCT | |
| Phenylketonuria; | ggccaaagcatgcatGAGCGAACTGAGAAGGGCTGGTTTCAGAGCT | 302 |
| not | ATGCTGGAAACAGCATAGCAAGTTGAAATAAGGCTAGTCCGTT | |
| provided | ATCAACTTGAAAAAGTGGCACCGAGTCGGTGCACAATACCTCG | |
| | GCCCTTCTCAGTTCAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTAAGCCTGTGGTTTTGG | |
| | TCTTAGGAACTTTGCTGCCACAATACCTCAGCCCTTCTCAGTT | |
| | CGCTACGACCCATATTGCATgctaagcAGCTTGGCGTAACTAGAT | |
| | CT | |
| Hyperphenyl | ggccaaagcatgcatGTCCCCCAATTACAGGAAGTGTTTCAGAGCTA | 303 |
| alaninemia, | TGCTGGAAACAGCATAGCAAGTTGAAATAAGGCTAGTCCGTTA | |
| non- | TCAACTTGAAAAAGTGGCACCGAGTCGGTGCCAATTTCCTGTA | |
| pku; | ATTGGGAAAAAAAAAGCTGCCATCAGTCGGCGTGGACTGTAG | |
| Phenylketonuria; | AACACTGCCAATGCCGGTCCCAAGCCCGGATAAAAGTGGAGG | |
| not provided | GTACAGTCCACGCTTTTTTTGTGGCGAGCTTTTCAATGTATTCA | |
| | TCAGGTGCACCCAGAGAGGCAAGGCCAACTTCCTGTAATTGG | |
| | GGGAAAATAGAACCGGTGAAgctaagcAGCTTGGCGTAACTAGA | |
| | TCT | |
| Hyperphenyl | ggccaaagcatgcatGTCCCCCAATTACAGGAAGTGTTTCAGAGCTA | 304 |
| alaninemia, | TGCTGGAAACAGCATAGCAAGTTGAAATAAGGCTAGTCCGTTA | |
| non- | TCAACTTGAAAAAGTGGCACCGAGTCGGTGCAGGCCAATTTC | |
| pku; | CTGTAATTGGGAAAAAAAAAGCTGCCATCAGTCGGCGTGGACT | |
| Phenylketonuria; | GTAGAACACTGCCAATGCCGGTCCCAAGCCCGGATAAAAGTG | |
| not provided | GAGGGTACAGTCCACGCTTTTTTTCGAGCTTTTCAATGTATTCA | |
| | TCAGGTGCACCCAGAGAGGCAAGGCCAACTTCCTGTAATTGG | |
| | GGGAAAATAGAACCAATGGAgctaagcAGCTTGGCGTAACTAGAT | |
| | CT | |
| Hyperphenyl | ggccaaagcatgcatGTCCCCCAATTACAGGAAGTGTTTCAGAGCTA | 305 |
| alaninemia, | TGCTGGAAACAGCATAGCAAGTTGAAATAAGGCTAGTCCGTTA | |
| non- | TCAACTTGAAAAAGTGGCACCGAGTCGGTGCGCAAGGCCAAT | |
| pku; | TTCCTGTAATTGGGAAAAAAAAAGCTGCCATCAGTCGGCGTGG | |
| Phenylketonuria; | ACTGTAGAACACTGCCAATGCCGGTCCCAAGCCCGGATAAAA | |
| not provided | GTGGAGGGTACAGTCCACGCTTTTTTTGCTTTTCAATGTATTCA | |
| | TCAGGTGCACCCAGAGAGGCAAGGCCAACTTCCTGTAATTGG | |
| | GGGAAAATAGAACCAGCGTAgctaagcAGCTTGGCGTAACTAGA | |
| | TCT | |
| Hyperphenyl | ggccaaagcatgcatGTCCCCCAATTACAGGAAGTGTTTCAGAGCTA | 306 |
| alaninemia, | TGCTGGAAACAGCATAGCAAGTTGAAATAAGGCTAGTCCGTTA | |
| non- | TCAACTTGAAAAAGTGGCACCGAGTCGGTGCGAGAGGCAAGG | |
| pku; | CCAATTTCCTGTAATTGGGAAAAAAAAAGCTGCCATCAGTCGG | |
| Phenylketonuria; | CGTGGACTGTAGAACACTGCCAATGCCGGTCCCAAGCCCGGA | |
| not provided | TAAAAGTGGAGGGTACAGTCCACGCTTTTTTTTCAATGTATTCA | |
| | TCAGGTGCACCCAGAGAGGCAAGGCCAACTTCCTGTAATTGG | |
| | GGGAAAATAGAACCATCTCCgctaagcAGCTTGGCGTAACTAGAT | |
| | CT | |
| Phenylketonuria; | ggccaaagcatgcatGTCTGATGTACTGTGTGCAGGTTTCAGAGCTA | 307 |
| not | TGCTGGAAACAGCATAGCAAGTTGAAATAAGGCTAGTCCGTTA | |
| provided | TCAACTTGAAAAAGTGGCACCGAGTCGGTGCTTCCGAGTCTTC | |
| | CACTGCACACAGTACATCAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTTCGGGATTTCTT | |
| | GGGTGGCCTGGCCTTCTGAGTCTTCCACTGCACACAGTACAT | |
| | CAGACATGGATCCAGCGGAGgctaagcAGCTTGGCGTAACTAGA | |
| | TCT | |
| Phenylketonuria; | ggccaaagcatgcatGTCTGATGTACTGTGTGCAGGTTTCAGAGCTA | 308 |
| not | TGCTGGAAACAGCATAGCAAGTTGAAATAAGGCTAGTCCGTTA | |
| provided | TCAACTTGAAAAAGTGGCACCGAGTCGGTGCGGCCTTCCGAG | |
| | TCTTCCACTGCACACAGTACATCAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTGATTTCT | |
| | TGGGTGGCCTGGCCTTCTGAGTCTTCCACTGCACACAGTACAT | |
| | CAGACATGGATCCACAGGCTgctaagcAGCTTGGCGTAACTAGA | |
| | TCT | |
| Phenylketonuria; | ggccaaagcatgcatGTCTGATGTACTGTGTGCAGGTTTCAGAGCTA | 309 |
| not | TGCTGGAAACAGCATAGCAAGTTGAAATAAGGCTAGTCCGTTA | |
| provided | TCAACTTGAAAAAGTGGCACCGAGTCGGTGCCCTGGCCTTCC | |
| | GAGTCTTCCACTGCACACAGTACATCAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTTTCT | |
| | TGGGTGGCCTGGCCTTCTGAGTCTTCCACTGCACACAGTACAT | |
| | CAGACATGGATCCAATGCCCgctaagcAGCTTGGCGTAACTAGAT | |
| | CT | |
| Phenylketonuria; | ggccaaagcatgcatGTCTGATGTACTGTGTGCAGGTTTCAGAGCTA | 310 |
| not | TGCTGGAAACAGCATAGCAAGTTGAAATAAGGCTAGTCCGTTA | |
| provided | TCAACTTGAAAAAGTGGCACCGAGTCGGTGCGGTGGCCTGGC | |
| | CTTCCGAGTCTTCCACTGCACACAGTACATCAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TGGGTGGCCTGGCCTTCTGAGTCTTCCACTGCACACAGTACAT | |
| | CAGACATGGATCCAAGATTTgctaagcAGCTTGGCGTAACTAGAT | |
| | CT | |
| Phenylketonuria; | ggccaaagcatgcatGGTGCAGTGGAAGACTCAGAGTTTCAGAGCT | 311 |
| not | ATGCTGGAAACAGCATAGCAAGTTGAAATAAGGCTAGTCCGTT | |
| provided | ATCAACTTGAAAAAGTGGCACCGAGTCGGTGCTTCCGAGTCTT | |
| | CCACTGAAAAAAAAAGCTGCCATCAGTCGGCGTGGACTGTAG | |
| | AACACTGCCAATGCCGGTCCCAAGCCCGGATAAAAGTGGAGG | |
| | GTACAGTCCACGCTTTTTTTCGACCTGTGGCTGGCCTGCTTTC | |
| | CTCTCGGGATTTCTTGGGTGGCCTGGCCTTCTGAGTCTTCCAC | |
| | TGCACACAGTACATCCGTGGTgctaagcAGCTTGGCGTAACTAGA | |
| | TCT | |
| Phenylketonuria; | ggccaaagcatgcatGGTGCAGTGGAAGACTCAGAGTTTCAGAGCT | 312 |
| not | ATGCTGGAAACAGCATAGCAAGTTGAAATAAGGCTAGTCCGTT | |
| provided | ATCAACTTGAAAAAGTGGCACCGAGTCGGTGCGGCCTTCCGA | |
| | GTCTTCCACTGAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTCTGTGGCTGGCCTGCTTTC | |
| | CTCTCGGGATTTCTTGGGTGGCCTGGCCTTCTGAGTCTTCCAC | |
| | TGCACACAGTACATCATGTTGgctaagcAGCTTGGCGTAACTAGA | |
| | TCT | |
| Phenylketonuria; | ggccaaagcatgcatGGTGCAGTGGAAGACTCAGAGTTTCAGAGCT | 313 |
| not | ATGCTGGAAACAGCATAGCAAGTTGAAATAAGGCTAGTCCGTT | |
| provided | ATCAACTTGAAAAAGTGGCACCGAGTCGGTGCCCTGGCCTTC | |
| | CGAGTCTTCCACTGAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTGGCTGGCCTGCTTT | |
| | CCTCTCGGGATTTCTTGGGTGGCCTGGCCTTCTGAGTCTTCCA | |
| | CTGCACACAGTACATCTTAGAGgctaagcAGCTTGGCGTAACTAG | |
| | ATCT | |
| Phenylketonuria; | ggccaaagcatgcatGGTGCAGTGGAAGACTCAGAGTTTCAGAGCT | 314 |
| not | ATGCTGGAAACAGCATAGCAAGTTGAAATAAGGCTAGTCCGTT | |
| provided | ATCAACTTGAAAAAGTGGCACCGAGTCGGTGCGGTGGCCTGG | |
| | CCTTCCGAGTCTTCCACTGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTGGCCTGCTTTC | |
| | CTCTCGGGATTTCTTGGGTGGCCTGGCCTTCTGAGTCTTCCAC | |
| | TGCACACAGTACATCTTCAGTgctaagcAGCTTGGCGTAACTAGA | |
| | TCT | |
| Phenylketonuria; | ggccaaagcatgcatGCGGGATTTCTTGGGTGGCCGTTTCAGAGCT | 315 |
| not | ATGCTGGAAACAGCATAGCAAGTTGAAATAAGGCTAGTCCGTT | |
| provided | ATCAACTTGAAAAAGTGGCACCGAGTCGGTGCCTCGGAAGGC | |
| | CAGGCCACCCAAGAAATCAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTATGTCTGATGT | |
| | ACTGTGTGCAGTGGAAGACTCAGAAGGCCAGGCCACCCAAGA | |
| | AATCCCGAGAGGAAAGCAAAGTCgctaagcAGCTTGGCGTAACT | |
| | AGATCT | |
| Phenylketonuria; | ggccaaagcatgcatGCGGGATTTCTTGGGTGGCCGTTTCAGAGCT | 316 |
| not | ATGCTGGAAACAGCATAGCAAGTTGAAATAAGGCTAGTCCGTT | |
| provided | ATCAACTTGAAAAAGTGGCACCGAGTCGGTGCAAGACTCGGA | |
| | AGGCCAGGCCACCCAAGAAATCAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTCTGATGT | |
| | ACTGTGTGCAGTGGAAGACTCAGAAGGCCAGGCCACCCAAGA | |
| | AATCCCGAGAGGAAAGCCTGGTCgctaagcAGCTTGGCGTAACT | |
| | AGATCT | |
| Phenylketonuria; | ggccaaagcatgcatGCGGGATTTCTTGGGTGGCCGTTTCAGAGCT | 317 |
| not | ATGCTGGAAACAGCATAGCAAGTTGAAATAAGGCTAGTCCGTT | |
| provided | ATCAACTTGAAAAAGTGGCACCGAGTCGGTGCTGGAAGACTC | |
| | GGAAGGCCAGGCCACCCAAGAAATCAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTATGT | |
| | ACTGTGTGCAGTGGAAGACTCAGAAGGCCAGGCCACCCAAGA | |
| | AATCCCGAGAGGAAAGCGTCCCTgctaagcAGCTTGGCGTAACT | |
| | AGATCT | |
| Phenylketonuria; | ggccaaagcatgcatGCGGGATTTCTTGGGTGGCCGTTTCAGAGCT | 318 |
| not | ATGCTGGAAACAGCATAGCAAGTTGAAATAAGGCTAGTCCGTT | |
| provided | ATCAACTTGAAAAAGTGGCACCGAGTCGGTGCTGCAGTGGAA | |
| | GACTCGGAAGGCCAGGCCACCCAAGAAATCAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TCTGTGTGCAGTGGAAGACTCAGAAGGCCAGGCCACCCAAGA | |
| | AATCCCGAGAGGAAAGCTAGTTCgctaagcAGCTTGGCGTAACTA | |
| | GATCT | |
| Inborn | ggccaaagcatgcatGTGAGCAGCTCAGGCTGCCGGTTTCAGAGCT | 319 |
| genetic | ATGCTGGAAACAGCATAGCAAGTTGAAATAAGGCTAGTCCGTT | |
| diseases; | ATCAACTTGAAAAAGTGGCACCGAGTCGGTGCTGAGCCACGG | |
| Phenylketonuria; | CAGCCTGAGCTGCAAAAAAAAAGCTGCCATCAGTCGGCGTGG | |
| not | ACTGTAGAACACTGCCAATGCCGGTCCCAAGCCCGGATAAAA | |
| provided | GTGGAGGGTACAGTCCACGCTTTTTTTACCCAGGCTTGGGCA | |
| | GGAAACTCTCTGACTTTGGACAGGTGATCCACGGCAGCCTGA | |
| | GCTGCTCAGTTAGGGGAAGAGGTGgctaagcAGCTTGGCGTAAC | |
| | TAGATCT | |
| Inborn | ggccaaagcatgcatGTGAGCAGCTCAGGCTGCCGGTTTCAGAGCT | 320 |
| genetic | ATGCTGGAAACAGCATAGCAAGTTGAAATAAGGCTAGTCCGTT | |
| diseases; | ATCAACTTGAAAAAGTGGCACCGAGTCGGTGCCAGGTGAGCC | |
| Phenylketonuria; | ACGGCAGCCTGAGCTGCAAAAAAAAAGCTGCCATCAGTCGGC | |
| not | GTGGACTGTAGAACACTGCCAATGCCGGTCCCAAGCCCGGAT | |
| provided | AAAAGTGGAGGGTACAGTCCACGCTTTTTTTAGGCTTGGGCAG | |
| | GAAACTCTCTGACTTTGGACAGGTGATCCACGGCAGCCTGAG | |
| | CTGCTCAGTTAGGGGAAATAGCCgctaagcAGCTTGGCGTAACT | |
| | AGATCT | |
| Inborn | ggccaaagcatgcatGTGAGCAGCTCAGGCTGCCGGTTTCAGAGCT | 321 |
| genetic | ATGCTGGAAACAGCATAGCAAGTTGAAATAAGGCTAGTCCGTT | |
| diseases; | ATCAACTTGAAAAAGTGGCACCGAGTCGGTGCGGACAGGTGA | |
| Phenylketonuria; | GCCACGGCAGCCTGAGCTGCAAAAAAAAAGCTGCCATCAGTC | |
| not | GGCGTGGACTGTAGAACACTGCCAATGCCGGTCCCAAGCCCG | |
| provided | GATAAAAGTGGAGGGTACAGTCCACGCTTTTTTTCTTGGGCAG | |
| | GAAACTCTCTGACTTTGGACAGGTGATCCACGGCAGCCTGAG | |
| | CTGCTCAGTTAGGGGAATCACTGgctaagcAGCTTGGCGTAACTA | |
| | GATCT | |
| Inborn | ggccaaagcatgcatGTGAGCAGCTCAGGCTGCCGGTTTCAGAGCT | 322 |
| genetic | ATGCTGGAAACAGCATAGCAAGTTGAAATAAGGCTAGTCCGTT | |
| diseases; | ATCAACTTGAAAAAGTGGCACCGAGTCGGTGCACTTTGGACA | |
| Phenylketonuria; | GGTGAGCCACGGCAGCCTGAGCTGCAAAAAAAAAGCTGCCAT | |
| not | CAGTCGGCGTGGACTGTAGAACACTGCCAATGCCGGTCCCAA | |
| provided | GCCCGGATAAAAGTGGAGGGTACAGTCCACGCTTTTTTTGCA | |
| | GGAAACTCTCTGACTTTGGACAGGTGATCCACGGCAGCCTGA | |
| | GCTGCTCAGTTAGGGGAATCCACTgctaagcAGCTTGGCGTAAC | |
| | TAGATCT | |
| Inborn | ggccaaagcatgcatGGCAGGAAACTCTCTGACTTGTTTCAGAGCTA | 323 |
| genetic | TGCTGGAAACAGCATAGCAAGTTGAAATAAGGCTAGTCCGTTA | |
| diseases; | TCAACTTGAAAAAGTGGCACCGAGTCGGTGCTGGCTCACCTG | |
| Phenylketonuria; | TCCAAAGTCAGAGAGTTTCCAAAAAAAAAGCTGCCATCAGTCG | |
| not | GCGTGGACTGTAGAACACTGCCAATGCCGGTCCCAAGCCCGG | |
| provided | ATAAAAGTGGAGGGTACAGTCCACGCTTTTTTTTAACTGAGCA | |
| | GCTCAGGCTGCCGTGGATCACCTGTCCAAAGTCAGAGAGTTT | |
| | CCTGCCCAAGCCTGGGATTTCgctaagcAGCTTGGCGTAACTAG | |
| | ATCT | |
| Inborn | ggccaaagcatgcatGGCAGGAAACTCTCTGACTTGTTTCAGAGCTA | 324 |
| genetic | TGCTGGAAACAGCATAGCAAGTTGAAATAAGGCTAGTCCGTTA | |
| diseases; | TCAACTTGAAAAAGTGGCACCGAGTCGGTGCGCCGTGGCTCA | |
| Phenylketonuria; | CCTGTCCAAAGTCAGAGAGTTTCCAAAAAAAAAGCTGCCATCA | |
| not | GTCGGCGTGGACTGTAGAACACTGCCAATGCCGGTCCCAAGC | |
| provided | CCGGATAAAAGTGGAGGGTACAGTCCACGCTTTTTTTTGAGCA | |
| | GCTCAGGCTGCCGTGGATCACCTGTCCAAAGTCAGAGAGTTT | |
| | CCTGCCCAAGCCTGGACGTCGgctaagcAGCTTGGCGTAACTAG | |
| | ATCT | |
| Inborn | ggccaaagcatgcatGGCAGGAAACTCTCTGACTTGTTTCAGAGCTA | 325 |
| genetic | TGCTGGAAACAGCATAGCAAGTTGAAATAAGGCTAGTCCGTTA | |
| diseases; | TCAACTTGAAAAAGTGGCACCGAGTCGGTGCGCTGCCGTGGC | |
| Phenylketonuria; | TCACCTGTCCAAAGTCAGAGAGTTTCCAAAAAAAAAGCTGCCA | |
| not | TCAGTCGGCGTGGACTGTAGAACACTGCCAATGCCGGTCCCA | |
| provided | AGCCCGGATAAAAGTGGAGGGTACAGTCCACGCTTTTTTTGCA | |
| | GCTCAGGCTGCCGTGGATCACCTGTCCAAAGTCAGAGAGTTT | |
| | CCTGCCCAAGCCTGGACTCGTgctaagcAGCTTGGCGTAACTAG | |
| | ATCT | |
| Inborn | ggccaaagcatgcatGAAACTCTCTGACTTTGGACGTTTCAGAGCTA | 326 |
| genetic | TGCTGGAAACAGCATAGCAAGTTGAAATAAGGCTAGTCCGTTA | |
| diseases; | TCAACTTGAAAAAGTGGCACCGAGTCGGTGCTGGCTCACCTG | |
| Phenylketonuria; | TCCAAAGTCAGAGAGAAAAAAAAAGCTGCCATCAGTCGGCGT | |
| not | GGACTGTAGAACACTGCCAATGCCGGTCCCAAGCCCGGATAA | |
| provided | AAGTGGAGGGTACAGTCCACGCTTTTTTTCAAATTCCCCTAAC | |
| | TGAGCAGCTCAGGCTGCCGTGGATCACCTGTCCAAAGTCAGA | |
| | GAGTTTCCTGCCCAAGGAGGAAgctaagcAGCTTGGCGTAACTA | |
| | GATCT | |
| Inborn | ggccaaagcatgcatGAAACTCTCTGACTTTGGACGTTTCAGAGCTA | 327 |
| genetic | TGCTGGAAACAGCATAGCAAGTTGAAATAAGGCTAGTCCGTTA | |
| diseases; | TCAACTTGAAAAAGTGGCACCGAGTCGGTGCGCCGTGGCTCA | |
| Phenylketonuria; | CCTGTCCAAAGTCAGAGAGAAAAAAAAAGCTGCCATCAGTCG | |
| not | GCGTGGACTGTAGAACACTGCCAATGCCGGTCCCAAGCCCGG | |
| provided | ATAAAAGTGGAGGGTACAGTCCACGCTTTTTTTTTCCCCTAACT | |
| | GAGCAGCTCAGGCTGCCGTGGATCACCTGTCCAAAGTCAGAG | |
| | AGTTTCCTGCCCAAGATTCCTgctaagcAGCTTGGCGTAACTAGA | |
| | TCT | |
| Inborn | ggccaaagcatgcatGAAACTCTCTGACTTTGGACGTTTCAGAGCTA | 328 |
| genetic | TGCTGGAAACAGCATAGCAAGTTGAAATAAGGCTAGTCCGTTA | |
| diseases; | TCAACTTGAAAAAGTGGCACCGAGTCGGTGCGCTGCCGTGGC | |
| Phenylketonuria; | TCACCTGTCCAAAGTCAGAGAGAAAAAAAAAGCTGCCATCAGT | |
| not | CGGCGTGGACTGTAGAACACTGCCAATGCCGGTCCCAAGCCC | |
| provided | GGATAAAAGTGGAGGGTACAGTCCACGCTTTTTTTCCCTAACT | |
| | GAGCAGCTCAGGCTGCCGTGGATCACCTGTCCAAAGTCAGAG | |
| | AGTTTCCTGCCCAAGCGCCTTgctaagcAGCTTGGCGTAACTAGA | |
| | TCT | |
| Inborn | ggccaaagcatgcatGAAACTCTCTGACTTTGGACGTTTCAGAGCTA | 329 |
| genetic | TGCTGGAAACAGCATAGCAAGTTGAAATAAGGCTAGTCCGTTA | |
| diseases; | TCAACTTGAAAAAGTGGCACCGAGTCGGTGCCTCAGGCTGCC | |
| Phenylketonuria; | GTGGCTCACCTGTCCAAAGTCAGAGAGAAAAAAAAAGCTGCC | |
| not | ATCAGTCGGCGTGGACTGTAGAACACTGCCAATGCCGGTCCC | |
| provided | AAGCCCGGATAAAAGTGGAGGGTACAGTCCACGCTTTTTTTAC | |
| | TGAGCAGCTCAGGCTGCCGTGGATCACCTGTCCAAAGTCAGA | |
| | GAGTTTCCTGCCCAAGTCGGGTgctaagcAGCTTGGCGTAACTA | |
| | GATCT | |
| Phenylketonuria; | ggccaaagcatgcatGCTTATCTCGTGAAAGCTCAGTTTCAGAGCTA | 330 |
| not | TGCTGGAAACAGCATAGCAAGTTGAAATAAGGCTAGTCCGTTA | |
| provided | TCAACTTGAAAAAGTGGCACCGAGTCGGTGCGTGCCACTGTC | |
| | CATGAGCTTTCACGAGATAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTTCATCAAGATCTT | |
| | GAGGCATGACATTGGTGACACTGTCCATGAGCTTTCACGAGAT | |
| | AAGAAGAAAGACAAACTAAgctaagcAGCTTGGCGTAACTAGATC | |
| | T | |
| Phenylketonuria; | ggccaaagcatgcatGCTTATCTCGTGAAAGCTCAGTTTCAGAGCTA | 331 |
| not | TGCTGGAAACAGCATAGCAAGTTGAAATAAGGCTAGTCCGTTA | |
| provided | TCAACTTGAAAAAGTGGCACCGAGTCGGTGCATTGGTGCCAC | |
| | TGTCCATGAGCTTTCACGAGATAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTCAAGATCT | |
| | TGAGGCATGACATTGGTGACACTGTCCATGAGCTTTCACGAGA | |
| | TAAGAAGAAAGACATCAGTTgctaagcAGCTTGGCGTAACTAGAT | |
| | CT | |
| Phenylketonuria; | ggccaaagcatgcatGCTTATCTCGTGAAAGCTCAGTTTCAGAGCTA | 332 |
| not | TGCTGGAAACAGCATAGCAAGTTGAAATAAGGCTAGTCCGTTA | |
| provided | TCAACTTGAAAAAGTGGCACCGAGTCGGTGCGACATTGGTGC | |
| | CACTGTCCATGAGCTTTCACGAGATAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTGATCT | |
| | TGAGGCATGACATTGGTGACACTGTCCATGAGCTTTCACGAGA | |
| | TAAGAAGAAAGACATTTCGGgctaagcAGCTTGGCGTAACTAGAT | |
| | CT | |
| Phenylketonuria; | ggccaaagcatgcatGCTTATCTCGTGAAAGCTCAGTTTCAGAGCTA | 333 |
| not | TGCTGGAAACAGCATAGCAAGTTGAAATAAGGCTAGTCCGTTA | |
| provided | TCAACTTGAAAAAGTGGCACCGAGTCGGTGCGGCATGACATT | |
| | GGTGCCACTGTCCATGAGCTTTCACGAGATAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | TGAGGCATGACATTGGTGACACTGTCCATGAGCTTTCACGAGA | |
| | TAAGAAGAAAGACAACGGTAgctaagcAGCTTGGCGTAACTAGAT | |
| | CT | |
| Phenylketonuria; | ggccaaagcatgcatGAGATCTTGAGGCATGACATGTTTCAGAGCTA | 334 |
| not | TGCTGGAAACAGCATAGCAAGTTGAAATAAGGCTAGTCCGTTA | |
| provided | TCAACTTGAAAAAGTGGCACCGAGTCGGTGCGTGGCACCAAT | |
| | GTCATGCCTCAAGAAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTGTCTTTCTTCTTATC | |
| | TCGTGAAAGCTCATGGACAGTGTCACCAATGTCATGCCTCAAG | |
| | ATCTTGATGATGTTGAGTTCgctaagcAGCTTGGCGTAACTAGAT | |
| | CT | |
| Phenylketonuria; | ggccaaagcatgcatGAGATCTTGAGGCATGACATGTTTCAGAGCTA | 335 |
| not | TGCTGGAAACAGCATAGCAAGTTGAAATAAGGCTAGTCCGTTA | |
| provided | TCAACTTGAAAAAGTGGCACCGAGTCGGTGCGACAGTGGCAC | |
| | CAATGTCATGCCTCAAGAAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTTTTCTTCTTATCT | |
| | CGTGAAAGCTCATGGACAGTGTCACCAATGTCATGCCTCAAGA | |
| | TCTTGATGATGTTGCTAGGgctaagcAGCTTGGCGTAACTAGATC | |
| | T | |
| Phenylketonuria; | ggccaaagcatgcatGAGATCTTGAGGCATGACATGTTTCAGAGCTA | 336 |
| not | TGCTGGAAACAGCATAGCAAGTTGAAATAAGGCTAGTCCGTTA | |
| provided | TCAACTTGAAAAAGTGGCACCGAGTCGGTGCATGGACAGTGG | |
| | CACCAATGTCATGCCTCAAGAAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCTTCTTATCT | |
| | CGTGAAAGCTCATGGACAGTGTCACCAATGTCATGCCTCAAGA | |
| | TCTTGATGATGTTTAGTTGgctaagcAGCTTGGCGTAACTAGATC | |
| | T | |
| Phenylketonuria; | ggccaaagcatgcatGAGATCTTGAGGCATGACATGTTTCAGAGCTA | 337 |
| not | TGCTGGAAACAGCATAGCAAGTTGAAATAAGGCTAGTCCGTTA | |
| provided | TCAACTTGAAAAAGTGGCACCGAGTCGGTGCAGCTCATGGAC | |
| | AGTGGCACCAATGTCATGCCTCAAGAAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTTATC | |
| | TCGTGAAAGCTCATGGACAGTGTCACCAATGTCATGCCTCAAG | |
| | ATCTTGATGATGTTCATTAGgctaagcAGCTTGGCGTAACTAGAT | |
| | CT | |
| Phenylketonuria; | ggccaaagcatgcatGGGGCAGCCCATCCCTCGAGGTTTCAGAGCT | 338 |
| not | ATGCTGGAAACAGCATAGCAAGTTGAAATAAGGCTAGTCCGTT | |
| provided | ATCAACTTGAAAAAGTGGCACCGAGTCGGTGCTATTCCACTCG | |
| | AGGGATGGGCTGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTTGAACACTGTGCCCCA | |
| | TGTTTTCTTTTCTTCCTCCATGTATCCCACTCGAGGGATGGGC | |
| | TGCCCACTAGAATACAGGGTGgctaagcAGCTTGGCGTAACTAG | |
| | ATCT | |
| Phenylketonuria; | ggccaaagcatgcatGGGGCAGCCCATCCCTCGAGGTTTCAGAGCT | 339 |
| not | ATGCTGGAAACAGCATAGCAAGTTGAAATAAGGCTAGTCCGTT | |
| provided | ATCAACTTGAAAAAGTGGCACCGAGTCGGTGCCATGTATTCCA | |
| | CTCGAGGGATGGGCTGAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTACACTGTGCCCCA | |
| | TGTTTTCTTTTCTTCCTCCATGTATCCCACTCGAGGGATGGGC | |
| | TGCCCACTAGAATACTATGTCgctaagcAGCTTGGCGTAACTAGA | |
| | TCT | |
| Phenylketonuria; | ggccaaagcatgcatGGGGCAGCCCATCCCTCGAGGTTTCAGAGCT | 340 |
| not | ATGCTGGAAACAGCATAGCAAGTTGAAATAAGGCTAGTCCGTT | |
| provided | ATCAACTTGAAAAAGTGGCACCGAGTCGGTGCCTCCATGTATT | |
| | CCACTCGAGGGATGGGCTGAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTCTGTGCCCCA | |
| | TGTTTTCTTTTCTTCCTCCATGTATCCCACTCGAGGGATGGGC | |
| | TGCCCACTAGAATACCTTCCAgctaagcAGCTTGGCGTAACTAGA | |
| | TCT | |
| Phenylketonuria; | ggccaaagcatgcatGGGGCAGCCCATCCCTCGAGGTTTCAGAGCT | 341 |
| not | ATGCTGGAAACAGCATAGCAAGTTGAAATAAGGCTAGTCCGTT | |
| provided | ATCAACTTGAAAAAGTGGCACCGAGTCGGTGCTCTTCCTCCAT | |
| | GTATTCCACTCGAGGGATGGGCTGAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTCCCCAT | |
| | GTTTTCTTTTCTTCCTCCATGTATCCCACTCGAGGGATGGGCT | |
| | GCCCACTAGAATACCAGAGTgctaagcAGCTTGGCGTAACTAGAT | |
| | CT | |
| Phenylketonuria; | ggccaaagcatgcatGGGCAGCCCATCCCTCGAGTGTTTCAGAGCT | 342 |
| not | ATGCTGGAAACAGCATAGCAAGTTGAAATAAGGCTAGTCCGTT | |
| provided | ATCAACTTGAAAAAGTGGCACCGAGTCGGTGCTCTTCCTCCAT | |
| | GTATTCCACTCGAGGGATGGGCTAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTTGCCCCA | |
| | TGTTTTCTTTTCTTCCTCCATGTATCCCACTCGAGGGATGGGC | |
| | TGCCCACTAGAATAACGGACgctaagcAGCTTGGCGTAACTAGAT | |
| | CT | |
| Phenylketonuria; | ggccaaagcatgcatGAGGAAGAAAAGAAAACATGGTTTCAGAGCTA | 343 |
| not | TGCTGGAAACAGCATAGCAAGTTGAAATAAGGCTAGTCCGTTA | |
| provided | TCAACTTGAAAAAGTGGCACCGAGTCGGTGCGCCCCATGTTTT | |
| | CTTTTCTTAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTGCAAGCATGGGTTTTATACAAG | |
| | GACTTCAGAGTCTTGAACACTGTGCCTCATGTTTTCTTTTCTTC | |
| | CTCCATGTATTCGTCACCgctaagcAGCTTGGCGTAACTAGATCT | |
| Phenylketonuria; | ggccaaagcatgcatGAGGAAGAAAAGAAAACATGGTTTCAGAGCTA | 344 |
| not | TGCTGGAAACAGCATAGCAAGTTGAAATAAGGCTAGTCCGTTA | |
| provided | TCAACTTGAAAAAGTGGCACCGAGTCGGTGCCTGTGCCCCAT | |
| | GTTTTCTTTTCTTAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTGCATGGGTTTTATACAA | |
| | GGACTTCAGAGTCTTGAACACTGTGCCTCATGTTTTCTTTTCTT | |
| | CCTCCATGTATTCCTAGGGgctaagcAGCTTGGCGTAACTAGATC | |
| | T | |
| Phenylketonuria; | ggccaaagcatgcatGTTTTCATCCCAGCTTGCACGTTTCAGAGCTA | 345 |
| not | TGCTGGAAACAGCATAGCAAGTTGAAATAAGGCTAGTCCGTTA | |
| provided | TCAACTTGAAAAAGTGGCACCGAGTCGGTGCAGGCGGAAACC | |
| | AGTGCAAGCTGGGATGAAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGAGAGGAAAGCA | |
| | GGCCAGCCACAGGTCGGAGGTGGAAACCAGTGCAAGCTGGG | |
| | ATGAAAAGAAGAAAGAACGGATGgctaagcAGCTTGGCGTAACTA | |
| | GATCT | |
| Phenylketonuria; | ggccaaagcatgcatGTTTTCATCCCAGCTTGCACGTTTCAGAGCTA | 346 |
| not | TGCTGGAAACAGCATAGCAAGTTGAAATAAGGCTAGTCCGTTA | |
| provided | TCAACTTGAAAAAGTGGCACCGAGTCGGTGCTCGGAGGCGGA | |
| | AACCAGTGCAAGCTGGGATGAAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTGGAAAGCAG | |
| | GCCAGCCACAGGTCGGAGGTGGAAACCAGTGCAAGCTGGGA | |
| | TGAAAAGAAGAAAGAACGAACGgctaagcAGCTTGGCGTAACTA | |
| | GATCT | |
| Phenylketonuria; | ggccaaagcatgcatGTTTTCATCCCAGCTTGCACGTTTCAGAGCTA | 347 |
| not | TGCTGGAAACAGCATAGCAAGTTGAAATAAGGCTAGTCCGTTA | |
| provided | TCAACTTGAAAAAGTGGCACCGAGTCGGTGCAGGTCGGAGGC | |
| | GGAAACCAGTGCAAGCTGGGATGAAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTAAGCA | |
| | GGCCAGCCACAGGTCGGAGGTGGAAACCAGTGCAAGCTGGG | |
| | ATGAAAAGAAGAAAGAAGCAGTTgctaagcAGCTTGGCGTAACTA | |
| | GATCT | |
| Phenylketonuria; | ggccaaagcatgcatGTTTTCATCCCAGCTTGCACGTTTCAGAGCTA | 348 |
| not | TGCTGGAAACAGCATAGCAAGTTGAAATAAGGCTAGTCCGTTA | |
| provided | TCAACTTGAAAAAGTGGCACCGAGTCGGTGCGCCACAGGTCG | |
| | GAGGCGGAAACCAGTGCAAGCTGGGATGAAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | GGCCAGCCACAGGTCGGAGGTGGAAACCAGTGCAAGCTGGG | |
| | ATGAAAAGAAGAAAGAACCTGATgctaagcAGCTTGGCGTAACTA | |
| | GATCT | |
| Phenylketonuria; | ggccaaagcatgcatGCCACCCAAGAAATCCCGAGGTTTCAGAGCTA | 349 |
| not | TGCTGGAAACAGCATAGCAAGTTGAAATAAGGCTAGTCCGTTA | |
| provided | TCAACTTGAAAAAGTGGCACCGAGTCGGTGCCTGCTTTCCTCT | |
| | CGGGATTTCTTGGGAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTGCACTGGTTTCCGC | |
| | CTCCGACCTGTGGCTGGCCTGTTTTCCTCTCGGGATTTCTTGG | |
| | GTGGCCTGGCCTTCCAAACAgctaagcAGCTTGGCGTAACTAGA | |
| | TCT | |
| Phenylketonuria; | ggccaaagcatgcatGCCACCCAAGAAATCCCGAGGTTTCAGAGCTA | 350 |
| not | TGCTGGAAACAGCATAGCAAGTTGAAATAAGGCTAGTCCGTTA | |
| provided | TCAACTTGAAAAAGTGGCACCGAGTCGGTGCTGGCCTGCTTT | |
| | CCTCTCGGGATTTCTTGGGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTCTGGTTTCCGC | |
| | CTCCGACCTGTGGCTGGCCTGTTTTCCTCTCGGGATTTCTTGG | |
| | GTGGCCTGGCCTTCCTCCCGgctaagcAGCTTGGCGTAACTAGA | |
| | TCT | |
| Phenylketonuria; | ggccaaagcatgcatGCCACCCAAGAAATCCCGAGGTTTCAGAGCTA | 351 |
| not | TGCTGGAAACAGCATAGCAAGTTGAAATAAGGCTAGTCCGTTA | |
| provided | TCAACTTGAAAAAGTGGCACCGAGTCGGTGCGGCTGGCCTGC | |
| | TTTCCTCTCGGGATTTCTTGGGAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTGTTTCCGC | |
| | CTCCGACCTGTGGCTGGCCTGTTTTCCTCTCGGGATTTCTTGG | |
| | GTGGCCTGGCCTTCACACAAgctaagcAGCTTGGCGTAACTAGA | |
| | TCT | |
| Phenylketonuria; | ggccaaagcatgcatGCCACCCAAGAAATCCCGAGGTTTCAGAGCTA | 352 |
| not | TGCTGGAAACAGCATAGCAAGTTGAAATAAGGCTAGTCCGTTA | |
| provided | TCAACTTGAAAAAGTGGCACCGAGTCGGTGCCCTGTGGCTGG | |
| | CCTGCTTTCCTCTCGGGATTTCTTGGGAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTCGC | |
| | CTCCGACCTGTGGCTGGCCTGTTTTCCTCTCGGGATTTCTTGG | |
| | GTGGCCTGGCCTTCGCGTGCgctaagcAGCTTGGCGTAACTAGA | |
| | TCT | |
| Phenylketonuria; | ggccaaagcatgcatGGAAATCCCGAGAGGAAAACGTTTCAGAGCTA | 353 |
| not | TGCTGGAAACAGCATAGCAAGTTGAAATAAGGCTAGTCCGTTA | |
| provided | TCAACTTGAAAAAGTGGCACCGAGTCGGTGCCTGCTTTCCTCT | |
| | CGGGATAAAAAAAAAGCTGCCATCAGTCGGCGTGGACTGTAG | |
| | AACACTGCCAATGCCGGTCCCAAGCCCGGATAAAAGTGGAGG | |
| | GTACAGTCCACGCTTTTTTTTCTTTTCATCCCAGCTTGCACTGG | |
| | TTTCCGCCTCCGACCTGTGGCTGGCCTGTTTTCCTCTCGGGAT | |
| | TTCTTGGGTGGCCAGGGAAgctaagcAGCTTGGCGTAACTAGAT | |
| | CT | |
| Phenylketonuria; | ggccaaagcatgcatGGAAATCCCGAGAGGAAAACGTTTCAGAGCTA | 354 |
| not | TGCTGGAAACAGCATAGCAAGTTGAAATAAGGCTAGTCCGTTA | |
| provided | TCAACTTGAAAAAGTGGCACCGAGTCGGTGCTGGCCTGCTTT | |
| | CCTCTCGGGATAAAAAAAAAGCTGCCATCAGTCGGCGTGGAC | |
| | TGTAGAACACTGCCAATGCCGGTCCCAAGCCCGGATAAAAGT | |
| | GGAGGGTACAGTCCACGCTTTTTTTTTCATCCCAGCTTGCACT | |
| | GGTTTCCGCCTCCGACCTGTGGCTGGCCTGTTTTCCTCTCGG | |
| | GATTTCTTGGGTGGCCGCTGGAgctaagcAGCTTGGCGTAACTA | |
| | GATCT | |
| Phenylketonuria; | ggccaaagcatgcatGGAAATCCCGAGAGGAAAACGTTTCAGAGCTA | 355 |
| not | TGCTGGAAACAGCATAGCAAGTTGAAATAAGGCTAGTCCGTTA | |
| provided | TCAACTTGAAAAAGTGGCACCGAGTCGGTGCGGCTGGCCTGC | |
| | TTTCCTCTCGGGATAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTATCCCAGCTTGCACTG | |
| | GTTTCCGCCTCCGACCTGTGGCTGGCCTGTTTTCCTCTCGGG | |
| | ATTTCTTGGGTGGCCTCTGTAgctaagcAGCTTGGCGTAACTAGA | |
| | TCT | |
| Phenylketonuria; | ggccaaagcatgcatGGAAATCCCGAGAGGAAAACGTTTCAGAGCTA | 356 |
| not | TGCTGGAAACAGCATAGCAAGTTGAAATAAGGCTAGTCCGTTA | |
| provided | TCAACTTGAAAAAGTGGCACCGAGTCGGTGCCCTGTGGCTGG | |
| | CCTGCTTTCCTCTCGGGATAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTAGCTTGCACTG | |
| | GTTTCCGCCTCCGACCTGTGGCTGGCCTGTTTTCCTCTCGGG | |
| | ATTTCTTGGGTGGCCGCAACCgctaagcAGCTTGGCGTAACTAG | |
| | ATCT | |
| Phenylketonuria; | ggccaaagcatgcatGTCCGCCTCCGACCTGTGGCGTTTCAGAGCT | 357 |
| not | ATGCTGGAAACAGCATAGCAAGTTGAAATAAGGCTAGTCCGTT | |
| provided | ATCAACTTGAAAAAGTGGCACCGAGTCGGTGCAAAGCAGGCC | |
| | AGCCACAGGTCGGAGGCAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTAAGGCCAGGCCA | |
| | CCCAAGAAATCCCGAGAGGAAAACAGGCCAGCCACAGGTCGG | |
| | AGGCGGAAACCAGTGCAAGACATgctaagcAGCTTGGCGTAACT | |
| | AGATCT | |
| Phenylketonuria; | ggccaaagcatgcatGTCCGCCTCCGACCTGTGGCGTTTCAGAGCT | 358 |
| not | ATGCTGGAAACAGCATAGCAAGTTGAAATAAGGCTAGTCCGTT | |
| provided | ATCAACTTGAAAAAGTGGCACCGAGTCGGTGCCGAGAGGAAA | |
| | GCAGGCCAGCCACAGGTCGGAGGCAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTGGCCA | |
| | CCCAAGAAATCCCGAGAGGAAAACAGGCCAGCCACAGGTCGG | |
| | AGGCGGAAACCAGTGCAGCTTAAgctaagcAGCTTGGCGTAACT | |
| | AGATCT | |
| Phenylketonuria; | ggccaaagcatgcatGGCTGGCCTGCTTTCCTCTCGTTTCAGAGCTA | 359 |
| not | TGCTGGAAACAGCATAGCAAGTTGAAATAAGGCTAGTCCGTTA | |
| provided | TCAACTTGAAAAAGTGGCACCGAGTCGGTGCTCCCGAGAGGA | |
| | AAGCAGGCCAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTTACTGTGTGCAGTGGAAGAC | |
| | TCGGAAGGCCAGGCCACCCAAGAAATCCTGAGAGGAAAGCAG | |
| | GCCAGCCACAGGTCGGCGATCTgctaagcAGCTTGGCGTAACTA | |
| | GATCT | |
| Phenylketonuria; | ggccaaagcatgcatGTTCCAGCCCCTCTATTACAGTTTCAGAGCTA | 360 |
| not | TGCTGGAAACAGCATAGCAAGTTGAAATAAGGCTAGTCCGTTA | |
| provided | TCAACTTGAAAAAGTGGCACCGAGTCGGTGCCCACGTAATAG | |
| | AGGGGCTGAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTGTCACCACCTCACCTTACTTTC | |
| | TCCTTGGCATCATTAAAACTCTCTGCCATGTAATAGAGGGGCT | |
| | GGAACTCCGTGACAACTAGGgctaagcAGCTTGGCGTAACTAGA | |
| | TCT | |
| Phenylketonuria; | ggccaaagcatgcatGTTCCAGCCCCTCTATTACAGTTTCAGAGCTA | 361 |
| not | TGCTGGAAACAGCATAGCAAGTTGAAATAAGGCTAGTCCGTTA | |
| provided | TCAACTTGAAAAAGTGGCACCGAGTCGGTGCTCTGCCACGTA | |
| | ATAGAGGGGCTGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTCCACCTCACCTTACTTT | |
| | CTCCTTGGCATCATTAAAACTCTCTGCCATGTAATAGAGGGGC | |
| | TGGAACTCCGTGACACACGTAgctaagcAGCTTGGCGTAACTAG | |
| | ATCT | |
| Phenylketonuria; | ggccaaagcatgcatGTTCCAGCCCCTCTATTACAGTTTCAGAGCTA | 362 |
| not | TGCTGGAAACAGCATAGCAAGTTGAAATAAGGCTAGTCCGTTA | |
| provided | TCAACTTGAAAAAGTGGCACCGAGTCGGTGCCTCTCTGCCAC | |
| | GTAATAGAGGGGCTGAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTCCTCACCTTACTTT | |
| | CTCCTTGGCATCATTAAAACTCTCTGCCATGTAATAGAGGGGC | |
| | TGGAACTCCGTGACAAGCCGGgctaagcAGCTTGGCGTAACTAG | |
| | ATCT | |
| Phenylketonuria; | ggccaaagcatgcatGTTCCAGCCCCTCTATTACAGTTTCAGAGCTA | 363 |
| not | TGCTGGAAACAGCATAGCAAGTTGAAATAAGGCTAGTCCGTTA | |
| provided | TCAACTTGAAAAAGTGGCACCGAGTCGGTGCTAAAACTCTCTG | |
| | CCACGTAATAGAGGGGCTGAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTCCTTACTTTCT | |
| | CCTTGGCATCATTAAAACTCTCTGCCATGTAATAGAGGGGCTG | |
| | GAACTCCGTGACAATGTCGgctaagcAGCTTGGCGTAACTAGATC | |
| | T | |
| Phenylketonuria; | ggccaaagcatgcatGAGCTGGAGGACAGTACTCAGTTTCAGAGCTA | 364 |
| not | TGCTGGAAACAGCATAGCAAGTTGAAATAAGGCTAGTCCGTTA | |
| provided | TCAACTTGAAAAAGTGGCACCGAGTCGGTGCACCGTGAGTAC | |
| | TGTCCTCCAAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTCACACAGTACATCAGACATGG | |
| | ATCCAAGCCCATGTATACCCCCGAACCATGAGTACTGTCCTCC | |
| | AGCTACCAGTTGCCTCTGTTgctaagcAGCTTGGCGTAACTAGAT | |
| | CT | |
| Phenylketonuria; | ggccaaagcatgcatGAGCTGGAGGACAGTACTCAGTTTCAGAGCTA | 365 |
| not | TGCTGGAAACAGCATAGCAAGTTGAAATAAGGCTAGTCCGTTA | |
| provided | TCAACTTGAAAAAGTGGCACCGAGTCGGTGCCCCCCGAACCG | |
| | TGAGTACTGTCCTCCAAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTTACATCAGACATGG | |
| | ATCCAAGCCCATGTATACCCCCGAACCATGAGTACTGTCCTCC | |
| | AGCTACCAGTTGCCCATTAAgctaagcAGCTTGGCGTAACTAGAT | |
| | CT | |
| Hyperphenyl | ggccaaagcatgcatGAGGACCGCAGTGGACACGCGTTTCAGAGCT | 366 |
| alaninemia, | ATGCTGGAAACAGCATAGCAAGTTGAAATAAGGCTAGTCCGTT | |
| non- | ATCAACTTGAAAAAGTGGCACCGAGTCGGTGCAGCATGTCCA | |
| pku; | CTGCGGTAAAAAAAAAGCTGCCATCAGTCGGCGTGGACTGTA | |
| Phenylketonuria; | GAACACTGCCAATGCCGGTCCCAAGCCCGGATAAAAGTGGAG | |
| not provided | GGTACAGTCCACGCTTTTTTTCTGCCTGTACCTGAGGCCCTAA | |
| | AAAGCCAGAGACCTCACTCCCGGGGAGCCAGCGTGTCCACTG | |
| | CGGTCCTGGAAAACCCAGCTTCTgctaagcAGCTTGGCGTAACTA | |
| | GATCT | |
| Hyperphenyl | ggccaaagcatgcatGAGGACCGCAGTGGACACGCGTTTCAGAGCT | 367 |
| alaninemia, | ATGCTGGAAACAGCATAGCAAGTTGAAATAAGGCTAGTCCGTT | |
| non- | ATCAACTTGAAAAAGTGGCACCGAGTCGGTGCAGCCAGCATG | |
| pku; | TCCACTGCGGTAAAAAAAAAGCTGCCATCAGTCGGCGTGGAC | |
| Phenylketonuria; | TGTAGAACACTGCCAATGCCGGTCCCAAGCCCGGATAAAAGT | |
| not provided | GGAGGGTACAGTCCACGCTTTTTTTCTGTACCTGAGGCCCTAA | |
| | AAAGCCAGAGACCTCACTCCCGGGGAGCCAGCGTGTCCACTG | |
| | CGGTCCTGGAAAACCCAGAATCCgctaagcAGCTTGGCGTAACT | |
| | AGATCT | |
| Hyperphenyl | ggccaaagcatgcatGAGGACCGCAGTGGACACGCGTTTCAGAGCT | 368 |
| alaninemia, | ATGCTGGAAACAGCATAGCAAGTTGAAATAAGGCTAGTCCGTT | |
| non- | ATCAACTTGAAAAAGTGGCACCGAGTCGGTGCGGGAGCCAGC | |
| pku; | ATGTCCACTGCGGTAAAAAAAAAGCTGCCATCAGTCGGCGTG | |
| Phenylketonuria; | GACTGTAGAACACTGCCAATGCCGGTCCCAAGCCCGGATAAA | |
| not provided | AGTGGAGGGTACAGTCCACGCTTTTTTTTACCTGAGGCCCTAA | |
| | AAAGCCAGAGACCTCACTCCCGGGGAGCCAGCGTGTCCACTG | |
| | CGGTCCTGGAAAACCCAGTCTAGgctaagcAGCTTGGCGTAACT | |
| | AGATCT | |
| Hyperphenyl | ggccaaagcatgcatGAGGACCGCAGTGGACACGCGTTTCAGAGCT | 369 |
| alaninemia, | ATGCTGGAAACAGCATAGCAAGTTGAAATAAGGCTAGTCCGTT | |
| non- | ATCAACTTGAAAAAGTGGCACCGAGTCGGTGCTCCCGGGGAG | |
| pku; | CCAGCATGTCCACTGCGGTAAAAAAAAAGCTGCCATCAGTCG | |
| Phenylketonuria; | GCGTGGACTGTAGAACACTGCCAATGCCGGTCCCAAGCCCGG | |
| not provided | ATAAAAGTGGAGGGTACAGTCCACGCTTTTTTTGAGGCCCTAA | |
| | AAAGCCAGAGACCTCACTCCCGGGGAGCCAGCGTGTCCACTG | |
| | CGGTCCTGGAAAACCCAATGAGAgctaagcAGCTTGGCGTAACT | |
| | AGATCT | |
| Hyperphenyl | ggccaaagcatgcatGAGCCAGAGACCTCACTCCCGTTTCAGAGCTA | 370 |
| alaninemia, | TGCTGGAAACAGCATAGCAAGTTGAAATAAGGCTAGTCCGTTA | |
| non- | TCAACTTGAAAAAGTGGCACCGAGTCGGTGCGTGGACATGCT | |
| pku; | GGCTCCCCGGGAGTGAGGTCTCTGAAAAAAAAAGCTGCCATC | |
| Phenylketonuria; | AGTCGGCGTGGACTGTAGAACACTGCCAATGCCGGTCCCAAG | |
| not provided | CCCGGATAAAAGTGGAGGGTACAGTCCACGCTTTTTTTTTTTC | |
| | CAGGACCGCAGTGGACACGCTGGCTCCCCGGGAGTGAGGTC | |
| | TCTGGCTTTTTAGGGCCAGACCTgctaagcAGCTTGGCGTAACTA | |
| | GATCT | |
| Hyperphenyl | ggccaaagcatgcatGAGCCAGAGACCTCACTCCCGTTTCAGAGCTA | 371 |
| alaninemia, | TGCTGGAAACAGCATAGCAAGTTGAAATAAGGCTAGTCCGTTA | |
| non- | TCAACTTGAAAAAGTGGCACCGAGTCGGTGCGCAGTGGACAT | |
| pku; | GCTGGCTCCCCGGGAGTGAGGTCTCTGAAAAAAAAAGCTGCC | |
| Phenylketonuria; | ATCAGTCGGCGTGGACTGTAGAACACTGCCAATGCCGGTCCC | |
| not provided | AAGCCCGGATAAAAGTGGAGGGTACAGTCCACGCTTTTTTTTC | |
| | CAGGACCGCAGTGGACACGCTGGCTCCCCGGGAGTGAGGTC | |
| | TCTGGCTTTTTAGGGCCTACTTCgctaagcAGCTTGGCGTAACTA | |
| | GATCT | |
| Hyperphenyl | ggccaaagcatgcatGGCCAGAGACCTCACTCCCGGTTTCAGAGCT | 372 |
| alaninemia, | ATGCTGGAAACAGCATAGCAAGTTGAAATAAGGCTAGTCCGTT | |
| non- | ATCAACTTGAAAAAGTGGCACCGAGTCGGTGCACATGCTGGC | |
| pku; | TCCCCGGGAGTGAGGTCTCTAAAAAAAAAGCTGCCATCAGTC | |
| Phenylketonuria; | GGCGTGGACTGTAGAACACTGCCAATGCCGGTCCCAAGCCCG | |
| not provided | GATAAAAGTGGAGGGTACAGTCCACGCTTTTTTTCCTGGGTTT | |
| | TCCAGGACCGCAGTGGACACGCTGGCTCCCCGGGAGTGAGG | |
| | TCTCTGGCTTTTTAGGGCCTGTCAgctaagcAGCTTGGCGTAACT | |
| | AGATCT | |
| Hyperphenyl | ggccaaagcatgcatGGCCAGAGACCTCACTCCCGGTTTCAGAGCT | 373 |
| alaninemia, | ATGCTGGAAACAGCATAGCAAGTTGAAATAAGGCTAGTCCGTT | |
| non- | ATCAACTTGAAAAAGTGGCACCGAGTCGGTGCGTGGACATGC | |
| pku; | TGGCTCCCCGGGAGTGAGGTCTCTAAAAAAAAAGCTGCCATC | |
| Phenylketonuria; | AGTCGGCGTGGACTGTAGAACACTGCCAATGCCGGTCCCAAG | |
| not provided | CCCGGATAAAAGTGGAGGGTACAGTCCACGCTTTTTTTGGTTT | |
| | TCCAGGACCGCAGTGGACACGCTGGCTCCCCGGGAGTGAGG | |
| | TCTCTGGCTTTTTAGGGCAAGCGCgctaagcAGCTTGGCGTAACT | |
| | AGATCT | |
| Hyperphenyl | ggccaaagcatgcatGGCCAGAGACCTCACTCCCGGTTTCAGAGCT | 374 |
| alaninemia, | ATGCTGGAAACAGCATAGCAAGTTGAAATAAGGCTAGTCCGTT | |
| non- | ATCAACTTGAAAAAGTGGCACCGAGTCGGTGCGGACCGCAGT | |
| pku; | GGACATGCTGGCTCCCCGGGAGTGAGGTCTCTAAAAAAAAAG | |
| Phenylketonuria; | CTGCCATCAGTCGGCGTGGACTGTAGAACACTGCCAATGCCG | |
| not provided | GTCCCAAGCCCGGATAAAAGTGGAGGGTACAGTCCACGCTTT | |
| | TTTTAGGACCGCAGTGGACACGCTGGCTCCCCGGGAGTGAGG | |
| | TCTCTGGCTTTTTAGGGCGATACAgctaagcAGCTTGGCGTAACT | |
| | AGATCT | |
| Argininosuccinate | ggccaaagcatgcatGGGCGTCATCTCTACGCTGTGTTTCAGAGCTA | 375 |
| lyase | TGCTGGAAACAGCATAGCAAGTTGAAATAAGGCTAGTCCGTTA | |
| deficiency; | TCAACTTGAAAAAGTGGCACCGAGTCGGTGCCCTGCAGCGTA | |
| not provided | GAGATGACAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTCCAGTGCCTGGGACCTAGGG | |
| | GAGGAGAAGCAGGGGGGTGATGTCTTGCCTACAGCGTAGAGA | |
| | TGACGCCAGTGGCCACCTTATCTgctaagcAGCTTGGCGTAACTA | |
| | GATCT | |
| Argininosuccinate | ggccaaagcatgcatGGGCGTCATCTCTACGCTGTGTTTCAGAGCTA | 376 |
| lyase | TGCTGGAAACAGCATAGCAAGTTGAAATAAGGCTAGTCCGTTA | |
| deficiency; | TCAACTTGAAAAAGTGGCACCGAGTCGGTGCCTTGCCTGCAG | |
| not provided | CGTAGAGATGACAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTGCCTGGGACCTAGGG | |
| | GAGGAGAAGCAGGGGGGTGATGTCTTGCCTACAGCGTAGAGA | |
| | TGACGCCAGTGGCCACCATCCGTgctaagcAGCTTGGCGTAACT | |
| | AGATCT | |
| Argininosuccinate | ggccaaagcatgcatGGGCGTCATCTCTACGCTGTGTTTCAGAGCTA | 377 |
| lyase | TGCTGGAAACAGCATAGCAAGTTGAAATAAGGCTAGTCCGTTA | |
| deficiency; | TCAACTTGAAAAAGTGGCACCGAGTCGGTGCTGTCTTGCCTG | |
| not provided | CAGCGTAGAGATGACAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTCTGGGACCTAGGG | |
| | GAGGAGAAGCAGGGGGGTGATGTCTTGCCTACAGCGTAGAGA | |
| | TGACGCCAGTGGCCACCTGACCTgctaagcAGCTTGGCGTAACT | |
| | AGATCT | |
| Argininosuccinate | ggccaaagcatgcatGGGCGTCATCTCTACGCTGTGTTTCAGAGCTA | 378 |
| lyase | TGCTGGAAACAGCATAGCAAGTTGAAATAAGGCTAGTCCGTTA | |
| deficiency; | TCAACTTGAAAAAGTGGCACCGAGTCGGTGCGGTGATGTCTT | |
| not provided | GCCTGCAGCGTAGAGATGACAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTACCTAGGGG | |
| | AGGAGAAGCAGGGGGGTGATGTCTTGCCTACAGCGTAGAGAT | |
| | GACGCCAGTGGCCACCAAGGTCgctaagcAGCTTGGCGTAACTA | |
| | GATCT | |
| Phenylketonuria; | ggccaaagcatgcatGGAGAGTTTTAATGATGCCAGTTTCAGAGCTA | 379 |
| not | TGCTGGAAACAGCATAGCAAGTTGAAATAAGGCTAGTCCGTTA | |
| provided | TCAACTTGAAAAAGTGGCACCGAGTCGGTGCCCTTACTTTCTC | |
| | CTTGGCATCATTAAAACTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGTGGCTCACCTT | |
| | TGTCACCACCTCACCTAACTTTCTCCTTGGCATCATTAAAACTC | |
| | TCTGCCACGTAAACCGATgctaagcAGCTTGGCGTAACTAGATCT | |
| Phenylketonuria; | ggccaaagcatgcatGGAGAGTTTTAATGATGCCAGTTTCAGAGCTA | 380 |
| not | TGCTGGAAACAGCATAGCAAGTTGAAATAAGGCTAGTCCGTTA | |
| provided | TCAACTTGAAAAAGTGGCACCGAGTCGGTGCCTCACCTTACTT | |
| | TCTCCTTGGCATCATTAAAACTAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCTCACCTTT | |
| | GTCACCACCTCACCTAACTTTCTCCTTGGCATCATTAAAACTCT | |
| | CTGCCACGTAACTCGTGgctaagcAGCTTGGCGTAACTAGATCT | |
| Phenylketonuria; | ggccaaagcatgcatGGAGAGTTTTAATGATGCCAGTTTCAGAGCTA | 381 |
| not | TGCTGGAAACAGCATAGCAAGTTGAAATAAGGCTAGTCCGTTA | |
| provided | TCAACTTGAAAAAGTGGCACCGAGTCGGTGCCACCTCACCTTA | |
| | CTTTCTCCTTGGCATCATTAAAACTAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTACCTTT | |
| | GTCACCACCTCACCTAACTTTCTCCTTGGCATCATTAAAACTCT | |
| | CTGCCACGTAAGAGTTGgctaagcAGCTTGGCGTAACTAGATCT | |
| Phenylketonuria; | ggccaaagcatgcatGGAGAGTTTTAATGATGCCAGTTTCAGAGCTA | 382 |
| not | TGCTGGAAACAGCATAGCAAGTTGAAATAAGGCTAGTCCGTTA | |
| provided | TCAACTTGAAAAAGTGGCACCGAGTCGGTGCGTCACCACCTC | |
| | ACCTTACTTTCTCCTTGGCATCATTAAAACTAAAAAAAAAGCTG | |
| | CCATCAGTCGGCGTGGACTGTAGAACACTGCCAATGCCGGTC | |
| | CCAAGCCCGGATAAAAGTGGAGGGTACAGTCCACGCTTTTTTT | |
| | TGTCACCACCTCACCTAACTTTCTCCTTGGCATCATTAAAACTC | |
| | TCTGCCACGTAACCTCCTgctaagcAGCTTGGCGTAACTAGATCT | |
| Phenylketonuria; | ggccaaagcatgcatGTGATGCCAAGGAGAAAGTTGTTTCAGAGCTA | 383 |
| not | TGCTGGAAACAGCATAGCAAGTTGAAATAAGGCTAGTCCGTTA | |
| provided | TCAACTTGAAAAAGTGGCACCGAGTCGGTGCCCTTACTTTCTC | |
| | CTTGGCAAAAAAAAAAGCTGCCATCAGTCGGCGTGGACTGTA | |
| | GAACACTGCCAATGCCGGTCCCAAGCCCGGATAAAAGTGGAG | |
| | GGTACAGTCCACGCTTTTTTTAGTCAGGAGGCCCCCAGAGCTA | |
| | GTGGCTCACCTTTGTCACCACCTCACCTAACTTTCTCCTTGGC | |
| | ATCATTAAAACTCTTATCTGgctaagcAGCTTGGCGTAACTAGATC | |
| | T | |
| Phenylketonuria; | ggccaaagcatgcatGTGATGCCAAGGAGAAAGTTGTTTCAGAGCTA | 384 |
| not | TGCTGGAAACAGCATAGCAAGTTGAAATAAGGCTAGTCCGTTA | |
| provided | TCAACTTGAAAAAGTGGCACCGAGTCGGTGCCACCTCACCTTA | |
| | CTTTCTCCTTGGCAAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTAGGCCCCCAGAGCTA | |
| | GTGGCTCACCTTTGTCACCACCTCACCTAACTTTCTCCTTGGC | |
| | ATCATTAAAACTCTAGATAAgctaagcAGCTTGGCGTAACTAGATC | |
| | T | |
| Phenylketonuria; | ggccaaagcatgcatGTGATGCCAAGGAGAAAGTTGTTTCAGAGCTA | 385 |
| not | TGCTGGAAACAGCATAGCAAGTTGAAATAAGGCTAGTCCGTTA | |
| provided | TCAACTTGAAAAAGTGGCACCGAGTCGGTGCGTCACCACCTC | |
| | ACCTTACTTTCTCCTTGGCAAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTCCCAGAGCTA | |
| | GTGGCTCACCTTTGTCACCACCTCACCTAACTTTCTCCTTGGC | |
| | ATCATTAAAACTCTCAACGGgctaagcAGCTTGGCGTAACTAGAT | |
| | CT | |
| Phenylketonuria; | ggccaaagcatgcatGAGGTGCACCCAGAGAGACAGTTTCAGAGCT | 386 |
| not | ATGCTGGAAACAGCATAGCAAGTTGAAATAAGGCTAGTCCGTT | |
| provided | ATCAACTTGAAAAAGTGGCACCGAGTCGGTGCTTGCCTCTCTG | |
| | GGTGCAAAAAAAAAAGCTGCCATCAGTCGGCGTGGACTGTAG | |
| | AACACTGCCAATGCCGGTCCCAAGCCCGGATAAAAGTGGAGG | |
| | GTACAGTCCACGCTTTTTTTATTACAGGAACAGAACAGGTTCTA | |
| | TTTTCCCCCAATTACAGGAAATTGGCCTTGTCTCTCTGGGTGC | |
| | ACCTGATGAATACACTATTCgctaagcAGCTTGGCGTAACTAGAT | |
| | CT | |
| Phenylketonuria; | ggccaaagcatgcatGAGGTGCACCCAGAGAGACAGTTTCAGAGCT | 387 |
| not | ATGCTGGAAACAGCATAGCAAGTTGAAATAAGGCTAGTCCGTT | |
| provided | ATCAACTTGAAAAAGTGGCACCGAGTCGGTGCGGCCTTGCCT | |
| | CTCTGGGTGCAAAAAAAAAAGCTGCCATCAGTCGGCGTGGAC | |
| | TGTAGAACACTGCCAATGCCGGTCCCAAGCCCGGATAAAAGT | |
| | GGAGGGTACAGTCCACGCTTTTTTTCAGGAACAGAACAGGTTC | |
| | TATTTTCCCCCAATTACAGGAAATTGGCCTTGTCTCTCTGGGT | |
| | GCACCTGATGAATACAATTTTCgctaagcAGCTTGGCGTAACTAG | |
| | ATCT | |
| Phenylketonuria; | ggccaaagcatgcatGAGGTGCACCCAGAGAGACAGTTTCAGAGCT | 388 |
| not | ATGCTGGAAACAGCATAGCAAGTTGAAATAAGGCTAGTCCGTT | |
| provided | ATCAACTTGAAAAAGTGGCACCGAGTCGGTGCATTGGCCTTG | |
| | CCTCTCTGGGTGCAAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGAACAGAACAGGTTC | |
| | TATTTTCCCCCAATTACAGGAAATTGGCCTTGTCTCTCTGGGT | |
| | GCACCTGATGAATACAATTAATgctaagcAGCTTGGCGTAACTAG | |
| | ATCT | |
| Phenylketonuria; | ggccaaagcatgcatGAGGTGCACCCAGAGAGACAGTTTCAGAGCT | 389 |
| not | ATGCTGGAAACAGCATAGCAAGTTGAAATAAGGCTAGTCCGTT | |
| provided | ATCAACTTGAAAAAGTGGCACCGAGTCGGTGCAGGAAATTGG | |
| | CCTTGCCTCTCTGGGTGCAAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTGAACAGGTTCT | |
| | ATTTTCCCCCAATTACAGGAAATTGGCCTTGTCTCTCTGGGTG | |
| | CACCTGATGAATACAAATAGTgctaagcAGCTTGGCGTAACTAGA | |
| | TCT | |
| Phenylketonuria; | ggccaaagcatgcatGTCCCCCAATTACAGGAAATGTTTCAGAGCTA | 390 |
| not | TGCTGGAAACAGCATAGCAAGTTGAAATAAGGCTAGTCCGTTA | |
| provided | TCAACTTGAAAAAGTGGCACCGAGTCGGTGCGAGGCAAGGCC | |
| | AATTTCCTGTAATTGGGAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTTTTCAATGTATTCA | |
| | TCAGGTGCACCCAGAGAGACAAGGCCAATTTCCTGTAATTGG | |
| | GGGAAAATAGAACCCGTTCGgctaagcAGCTTGGCGTAACTAGA | |
| | TCT | |
| Phenylketonuria; | ggccaaagcatgcatGTCCCCCAATTACAGGAAATGTTTCAGAGCTA | 391 |
| not | TGCTGGAAACAGCATAGCAAGTTGAAATAAGGCTAGTCCGTTA | |
| provided | TCAACTTGAAAAAGTGGCACCGAGTCGGTGCCAGAGAGGCAA | |
| | GGCCAATTTCCTGTAATTGGGAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTAATGTATTCA | |
| | TCAGGTGCACCCAGAGAGACAAGGCCAATTTCCTGTAATTGG | |
| | GGGAAAATAGAACCGACTCCgctaagcAGCTTGGCGTAACTAGA | |
| | TCT | |
| Phenylketonuria; | ggccaaagcatgcatGTCCCCCAATTACAGGAAATGTTTCAGAGCTA | 392 |
| not | TGCTGGAAACAGCATAGCAAGTTGAAATAAGGCTAGTCCGTTA | |
| provided | TCAACTTGAAAAAGTGGCACCGAGTCGGTGCACCCAGAGAGG | |
| | CAAGGCCAATTTCCTGTAATTGGGAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTGTATTC | |
| | ATCAGGTGCACCCAGAGAGACAAGGCCAATTTCCTGTAATTGG | |
| | GGGAAAATAGAACCTCCTTTgctaagcAGCTTGGCGTAACTAGAT | |
| | CT | |
| Phenylketonuria; | ggccaaagcatgcatGTCCCCCAATTACAGGAAATGTTTCAGAGCTA | 393 |
| not | TGCTGGAAACAGCATAGCAAGTTGAAATAAGGCTAGTCCGTTA | |
| provided | TCAACTTGAAAAAGTGGCACCGAGTCGGTGCGGTGCACCCAG | |
| | AGAGGCAAGGCCAATTTCCTGTAATTGGGAAAAAAAAAGCTGC | |
| | CATCAGTCGGCGTGGACTGTAGAACACTGCCAATGCCGGTCC | |
| | CAAGCCCGGATAAAAGTGGAGGGTACAGTCCACGCTTTTTTTC | |
| | ATCAGGTGCACCCAGAGAGACAAGGCCAATTTCCTGTAATTGG | |
| | GGGAAAATAGAACCGCTAAAgctaagcAGCTTGGCGTAACTAGAT | |
| | CT | |
| Phenylketonuria; | ggccaaagcatgcatGAAAAAATCCATTCCTTATCGTTTCAGAGCTAT | 394 |
| not | GCTGGAAACAGCATAGCAAGTTGAAATAAGGCTAGTCCGTTAT | |
| provided | CAACTTGAAAAAGTGGCACCGAGTCGGTGCCAGGTAAGGAAT | |
| | GGATTTAAAAAAAAAGCTGCCATCAGTCGGCGTGGACTGTAGA | |
| | ACACTGCCAATGCCGGTCCCAAGCCCGGATAAAAGTGGAGGG | |
| | TACAGTCCACGCTTTTTTTGTTGGGACATGTGCCCTTGTTTTCA | |
| | GATCGCAGCTTTGCCCAGTTTTCCCAGATAAGGAATGGATTTT | |
| | TTAGCCTTCTAGCCATCAgctaagcAGCTTGGCGTAACTAGATCT | |
| Phenylketonuria; | ggccaaagcatgcatGAAAAAATCCATTCCTTATCGTTTCAGAGCTAT | 395 |
| not | GCTGGAAACAGCATAGCAAGTTGAAATAAGGCTAGTCCGTTAT | |
| provided | CAACTTGAAAAAGTGGCACCGAGTCGGTGCTTCCCAGGTAAG | |
| | GAATGGATTTAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTGGACATGTGCCCTTGTTTTC | |
| | AGATCGCAGCTTTGCCCAGTTTTCCCAGATAAGGAATGGATTT | |
| | TTTAGCCTTCTAGGGGTGAgctaagcAGCTTGGCGTAACTAGATC | |
| | T | |
| Phenylketonuria; | ggccaaagcatgcatGAAAAAATCCATTCCTTATCGTTTCAGAGCTAT | 396 |
| not | GCTGGAAACAGCATAGCAAGTTGAAATAAGGCTAGTCCGTTAT | |
| provided | CAACTTGAAAAAGTGGCACCGAGTCGGTGCGTTTTCCCAGGT | |
| | AAGGAATGGATTTAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTCATGTGCCCTTGTTTTC | |
| | AGATCGCAGCTTTGCCCAGTTTTCCCAGATAAGGAATGGATTT | |
| | TTTAGCCTTCTAGCATCACgctaagcAGCTTGGCGTAACTAGATC | |
| | T | |
| Phenylketonuria; | ggccaaagcatgcatGAAAAAATCCATTCCTTATCGTTTCAGAGCTAT | 397 |
| not | GCTGGAAACAGCATAGCAAGTTGAAATAAGGCTAGTCCGTTAT | |
| provided | CAACTTGAAAAAGTGGCACCGAGTCGGTGCGCCCAGTTTTCC | |
| | CAGGTAAGGAATGGATTTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGCCCTTGTTTTC | |
| | AGATCGCAGCTTTGCCCAGTTTTCCCAGATAAGGAATGGATTT | |
| | TTTAGCCTTCTAGGTCCTCgctaagcAGCTTGGCGTAACTAGATC | |
| | T | |
| Phenylketonuria; | ggccaaagcatgcatGAAAAATCCATTCCTTATCTGTTTCAGAGCTAT | 398 |
| not | GCTGGAAACAGCATAGCAAGTTGAAATAAGGCTAGTCCGTTAT | |
| provided | CAACTTGAAAAAGTGGCACCGAGTCGGTGCCAGGTAAGGAAT | |
| | GGATTAAAAAAAAAGCTGCCATCAGTCGGCGTGGACTGTAGAA | |
| | CACTGCCAATGCCGGTCCCAAGCCCGGATAAAAGTGGAGGGT | |
| | ACAGTCCACGCTTTTTTTCTGTTGGGACATGTGCCCTTGTTTTC | |
| | AGATCGCAGCTTTGCCCAGTTTTCCCAGATAAGGAATGGATTT | |
| | TTTAGCCTTCTATTCGAAgctaagcAGCTTGGCGTAACTAGATCT | |
| Phenylketonuria; | ggccaaagcatgcatGAAAAATCCATTCCTTATCTGTTTCAGAGCTAT | 399 |
| not | GCTGGAAACAGCATAGCAAGTTGAAATAAGGCTAGTCCGTTAT | |
| provided | CAACTTGAAAAAGTGGCACCGAGTCGGTGCTTCCCAGGTAAG | |
| | GAATGGATTAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTTGGGACATGTGCCCTTGTTTT | |
| | CAGATCGCAGCTTTGCCCAGTTTTCCCAGATAAGGAATGGATT | |
| | TTTTAGCCTTCTAGCCAGGgctaagcAGCTTGGCGTAACTAGATC | |
| | T | |
| Phenylketonuria; | ggccaaagcatgcatGAAAAATCCATTCCTTATCTGTTTCAGAGCTAT | 400 |
| not | GCTGGAAACAGCATAGCAAGTTGAAATAAGGCTAGTCCGTTAT | |
| provided | CAACTTGAAAAAGTGGCACCGAGTCGGTGCGTTTTCCCAGGT | |
| | AAGGAATGGATTAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTGACATGTGCCCTTGTTT | |
| | TCAGATCGCAGCTTTGCCCAGTTTTCCCAGATAAGGAATGGAT | |
| | TTTTTAGCCTTCTAGAGGGAgctaagcAGCTTGGCGTAACTAGAT | |
| | CT | |
| Phenylketonuria; | ggccaaagcatgcatGAAAAATCCATTCCTTATCTGTTTCAGAGCTAT | 401 |
| not | GCTGGAAACAGCATAGCAAGTTGAAATAAGGCTAGTCCGTTAT | |
| provided | CAACTTGAAAAAGTGGCACCGAGTCGGTGCGCCCAGTTTTCC | |
| | CAGGTAAGGAATGGATTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGTGCCCTTGTTT | |
| | TCAGATCGCAGCTTTGCCCAGTTTTCCCAGATAAGGAATGGAT | |
| | TTTTTAGCCTTCTAATCAAGgctaagcAGCTTGGCGTAACTAGATC | |
| | T | |
| Phenylketonuria; | ggccaaagcatgcatGTGCCCAGTTTTCCCAGATAGTTTCAGAGCTA | 402 |
| not | TGCTGGAAACAGCATAGCAAGTTGAAATAAGGCTAGTCCGTTA | |
| provided | TCAACTTGAAAAAGTGGCACCGAGTCGGTGCTTACCTGGGAA | |
| | AACTGGAAAAAAAAAGCTGCCATCAGTCGGCGTGGACTGTAG | |
| | AACACTGCCAATGCCGGTCCCAAGCCCGGATAAAAGTGGAGG | |
| | GTACAGTCCACGCTTTTTTTTGAGAAATTCAGGTCACAGACCT | |
| | ATAACTAGAAGGCTAAAAAATCCATTCCTTATCTGGGAAAACTG | |
| | GGCAAAGCTGCGATTCCAGTgctaagcAGCTTGGCGTAACTAGA | |
| | TCT | |
| Phenylketonuria; | ggccaaagcatgcatGTGCCCAGTTTTCCCAGATAGTTTCAGAGCTA | 403 |
| not | TGCTGGAAACAGCATAGCAAGTTGAAATAAGGCTAGTCCGTTA | |
| provided | TCAACTTGAAAAAGTGGCACCGAGTCGGTGCTTCCTTACCTGG | |
| | GAAAACTGGAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTAAATTCAGGTCACAGACCTA | |
| | TAACTAGAAGGCTAAAAAATCCATTCCTTATCTGGGAAAACTG | |
| | GGCAAAGCTGCGATGAGCCGgctaagcAGCTTGGCGTAACTAGA | |
| | TCT | |
| Phenylketonuria; | ggccaaagcatgcatGTGCCCAGTTTTCCCAGATAGTTTCAGAGCTA | 404 |
| not | TGCTGGAAACAGCATAGCAAGTTGAAATAAGGCTAGTCCGTTA | |
| provided | TCAACTTGAAAAAGTGGCACCGAGTCGGTGCCCATTCCTTACC | |
| | TGGGAAAACTGGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTTTCAGGTCACAGACCTA | |
| | TAACTAGAAGGCTAAAAAATCCATTCCTTATCTGGGAAAACTG | |
| | GGCAAAGCTGCGATGGTGTTgctaagcAGCTTGGCGTAACTAGA | |
| | TCT | |
| Phenylketonuria; | ggccaaagcatgcatGTGCCCAGTTTTCCCAGATAGTTTCAGAGCTA | 405 |
| not | TGCTGGAAACAGCATAGCAAGTTGAAATAAGGCTAGTCCGTTA | |
| provided | TCAACTTGAAAAAGTGGCACCGAGTCGGTGCAAAATCCATTCC | |
| | TTACCTGGGAAAACTGGAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGTCACAGACCTA | |
| | TAACTAGAAGGCTAAAAAATCCATTCCTTATCTGGGAAAACTG | |
| | GGCAAAGCTGCGATTAGGACgctaagcAGCTTGGCGTAACTAGA | |
| | TCT | |
| Phenylketonuria; | ggccaaagcatgcatGGGGCTTGGATCCATGTCTGGTTTCAGAGCTA | 406 |
| not | TGCTGGAAACAGCATAGCAAGTTGAAATAAGGCTAGTCCGTTA | |
| provided | TCAACTTGAAAAAGTGGCACCGAGTCGGTGCTACATCAGACAT | |
| | GGATCCAAGAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTTTTCTTGGGTGGCCTGGCCT | |
| | TCCGAGTCTTCCACTGCACACAGTACCTCAGACATGGATCCAA | |
| | GCCCATGTATACCCGTCAGCgctaagcAGCTTGGCGTAACTAGAT | |
| | CT | |
| Phenylketonuria; | ggccaaagcatgcatGGGGCTTGGATCCATGTCTGGTTTCAGAGCTA | 407 |
| not | TGCTGGAAACAGCATAGCAAGTTGAAATAAGGCTAGTCCGTTA | |
| provided | TCAACTTGAAAAAGTGGCACCGAGTCGGTGCACAGTACATCA | |
| | GACATGGATCCAAGAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTTGGGTGGCCTGGC | |
| | CTTCCGAGTCTTCCACTGCACACAGTACCTCAGACATGGATCC | |
| | AAGCCCATGTATACCCTTCCCTgctaagcAGCTTGGCGTAACTAG | |
| | ATCT | |
| Phenylketonuria; | ggccaaagcatgcatGGGGCTTGGATCCATGTCTGGTTTCAGAGCTA | 408 |
| not | TGCTGGAAACAGCATAGCAAGTTGAAATAAGGCTAGTCCGTTA | |
| provided | TCAACTTGAAAAAGTGGCACCGAGTCGGTGCCACACAGTACAT | |
| | CAGACATGGATCCAAGAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTGGTGGCCTGGCC | |
| | TTCCGAGTCTTCCACTGCACACAGTACCTCAGACATGGATCCA | |
| | AGCCCATGTATACCCGTATTCgctaagcAGCTTGGCGTAACTAGA | |
| | TCT | |
| Phenylketonuria; | ggccaaagcatgcatGGGGCTTGGATCCATGTCTGGTTTCAGAGCTA | 409 |
| not | TGCTGGAAACAGCATAGCAAGTTGAAATAAGGCTAGTCCGTTA | |
| provided | TCAACTTGAAAAAGTGGCACCGAGTCGGTGCCACTGCACACA | |
| | GTACATCAGACATGGATCCAAGAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTCCTGGCCT | |
| | TCCGAGTCTTCCACTGCACACAGTACCTCAGACATGGATCCAA | |
| | GCCCATGTATACCCGTTGCCgctaagcAGCTTGGCGTAACTAGAT | |
| | CT | |
| Phenylketonuria; | ggccaaagcatgcatGGGAAGCCACAGTACTTTTCGTTTCAGAGCTA | 410 |
| not | TGCTGGAAACAGCATAGCAAGTTGAAATAAGGCTAGTCCGTTA | |
| provided | TCAACTTGAAAAAGTGGCACCGAGTCGGTGCTTCTTGAAAAGT | |
| | ACTGTGGCTAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTTATAAAACCCATGCTTGCTAT | |
| | GAGTACAATCACATTTTTCCACTTCCTGAAAAGTACTGTGGCTT | |
| | CCATGAAGATAACCTTACgctaagcAGCTTGGCGTAACTAGATCT | |
| Phenylketonuria; | ggccaaagcatgcatGGGAAGCCACAGTACTTTTCGTTTCAGAGCTA | 411 |
| not | TGCTGGAAACAGCATAGCAAGTTGAAATAAGGCTAGTCCGTTA | |
| provided | TCAACTTGAAAAAGTGGCACCGAGTCGGTGCCCACTTCTTGAA | |
| | AAGTACTGTGGCTAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTAAACCCATGCTTGCTA | |
| | TGAGTACAATCACATTTTTCCACTTCCTGAAAAGTACTGTGGCT | |
| | TCCATGAAGATAAGCTCGGgctaagcAGCTTGGCGTAACTAGATC | |
| | T | |
| Phenylketonuria; | ggccaaagcatgcatGGGAAGCCACAGTACTTTTCGTTTCAGAGCTA | 412 |
| not | TGCTGGAAACAGCATAGCAAGTTGAAATAAGGCTAGTCCGTTA | |
| provided | TCAACTTGAAAAAGTGGCACCGAGTCGGTGCTTTCCACTTCTT | |
| | GAAAAGTACTGTGGCTAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTCCCATGCTTGCTAT | |
| | GAGTACAATCACATTTTTCCACTTCCTGAAAAGTACTGTGGCTT | |
| | CCATGAAGATAACCATTGgctaagcAGCTTGGCGTAACTAGATCT | |
| Phenylketonuria; | ggccaaagcatgcatGGGAAGCCACAGTACTTTTCGTTTCAGAGCTA | 413 |
| not | TGCTGGAAACAGCATAGCAAGTTGAAATAAGGCTAGTCCGTTA | |
| provided | TCAACTTGAAAAAGTGGCACCGAGTCGGTGCACATTTTTCCAC | |
| | TTCTTGAAAAGTACTGTGGCTAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTGCTTGCTAT | |
| | GAGTACAATCACATTTTTCCACTTCCTGAAAAGTACTGTGGCTT | |
| | CCATGAAGATAACGAAGCgctaagcAGCTTGGCGTAACTAGATCT | |
| Argininosuccinate | ggccaaagcatgcatGCATTAGGACCATGGTGGATGTTTCAGAGCTA | 414 |
| lyase | TGCTGGAAACAGCATAGCAAGTTGAAATAAGGCTAGTCCGTTA | |
| deficiency; | TCAACTTGAAAAAGTGGCACCGAGTCGGTGCTCACGCCTCTG | |
| not provided | CCCGATCCACCATGGTCCTAAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTCCCCCTGGGT | |
| | GTCCCTGTAGGACTCATGCCTCTGCCCGATCCACCATGGTCC | |
| | TAATGAGCTCCCAGAACCTGTgctaagcAGCTTGGCGTAACTAGA | |
| | TCT | |
| Argininosuccinate | ggccaaagcatgcatGCATTAGGACCATGGTGGATGTTTCAGAGCTA | 415 |
| lyase | TGCTGGAAACAGCATAGCAAGTTGAAATAAGGCTAGTCCGTTA | |
| deficiency; | TCAACTTGAAAAAGTGGCACCGAGTCGGTGCGGACTCACGCC | |
| not provided | TCTGCCCGATCCACCATGGTCCTAAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTCTGGGT | |
| | GTCCCTGTAGGACTCATGCCTCTGCCCGATCCACCATGGTCC | |
| | TAATGAGCTCCCAGATATTTCgctaagcAGCTTGGCGTAACTAGA | |
| | TCT | |
| Argininosuccinate | ggccaaagcatgcatGCATTAGGACCATGGTGGATGTTTCAGAGCTA | 416 |
| lyase | TGCTGGAAACAGCATAGCAAGTTGAAATAAGGCTAGTCCGTTA | |
| deficiency; | TCAACTTGAAAAAGTGGCACCGAGTCGGTGCGTAGGACTCAC | |
| not provided | GCCTCTGCCCGATCCACCATGGTCCTAAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTGGT | |
| | GTCCCTGTAGGACTCATGCCTCTGCCCGATCCACCATGGTCC | |
| | TAATGAGCTCCCAGAGGCGAGgctaagcAGCTTGGCGTAACTAG | |
| | ATCT | |
| Argininosuccinate | ggccaaagcatgcatGATTAGGACCATGGTGGATCGTTTCAGAGCTA | 417 |
| lyase | TGCTGGAAACAGCATAGCAAGTTGAAATAAGGCTAGTCCGTTA | |
| deficiency; | TCAACTTGAAAAAGTGGCACCGAGTCGGTGCTCACGCCTCTG | |
| not provided | CCCGATCCACCATGGTCCTAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTTGCCCCCTGGG | |
| | TGTCCCTGTAGGACTCATGCCTCTGCCCGATCCACCATGGTC | |
| | CTAATGAGCTCCCAGGCGGTAgctaagcAGCTTGGCGTAACTAG | |
| | ATCT | |
| Argininosuccinate | ggccaaagcatgcatGATTAGGACCATGGTGGATCGTTTCAGAGCTA | 418 |
| lyase | TGCTGGAAACAGCATAGCAAGTTGAAATAAGGCTAGTCCGTTA | |
| deficiency; | TCAACTTGAAAAAGTGGCACCGAGTCGGTGCGGACTCACGCC | |
| not provided | TCTGCCCGATCCACCATGGTCCTAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTCCCTGGG | |
| | TGTCCCTGTAGGACTCATGCCTCTGCCCGATCCACCATGGTC | |
| | CTAATGAGCTCCCAGAGAGAGgctaagcAGCTTGGCGTAACTAG | |
| | ATCT | |
| Argininosuccinate | ggccaaagcatgcatGATTAGGACCATGGTGGATCGTTTCAGAGCTA | 419 |
| lyase | TGCTGGAAACAGCATAGCAAGTTGAAATAAGGCTAGTCCGTTA | |
| deficiency; | TCAACTTGAAAAAGTGGCACCGAGTCGGTGCGTAGGACTCAC | |
| not provided | GCCTCTGCCCGATCCACCATGGTCCTAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTTGG | |
| | GTGTCCCTGTAGGACTCATGCCTCTGCCCGATCCACCATGGT | |
| | CCTAATGAGCTCCCAGGGTTGAgctaagcAGCTTGGCGTAACTA | |
| | GATCT | |
| Argininosuccinate | ggccaaagcatgcatGATTAGGACCATGGTGGATCGTTTCAGAGCTA | 420 |
| lyase | TGCTGGAAACAGCATAGCAAGTTGAAATAAGGCTAGTCCGTTA | |
| deficiency; | TCAACTTGAAAAAGTGGCACCGAGTCGGTGCTCCCTGTAGGA | |
| not provided | CTCACGCCTCTGCCCGATCCACCATGGTCCTAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TGTCCCTGTAGGACTCATGCCTCTGCCCGATCCACCATGGTC | |
| | CTAATGAGCTCCCAGCACTAGgctaagcAGCTTGGCGTAACTAGA | |
| | TCT | |
| Argininosuccinate | ggccaaagcatgcatGGCCCCCTGGGTGTCCCTGTGTTTCAGAGCT | 421 |
| lyase | ATGCTGGAAACAGCATAGCAAGTTGAAATAAGGCTAGTCCGTT | |
| deficiency; | ATCAACTTGAAAAAGTGGCACCGAGTCGGTGCGGCGTGAGTC | |
| not provided | CTACAGGGACACCCAGGGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTCTCATTAGGAC | |
| | CATGGTGGATCGGGCAGAGGCATGAGTCCTACAGGGACACCC | |
| | AGGGGGCAGACAGAGGTTCTTTTgctaagcAGCTTGGCGTAACT | |
| | AGATCT | |
| Argininosuccinate | ggccaaagcatgcatGGCCCCCTGGGTGTCCCTGTGTTTCAGAGCT | 422 |
| lyase | ATGCTGGAAACAGCATAGCAAGTTGAAATAAGGCTAGTCCGTT | |
| deficiency; | ATCAACTTGAAAAAGTGGCACCGAGTCGGTGCCAGAGGCGTG | |
| not provided | AGTCCTACAGGGACACCCAGGGAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTTTAGGAC | |
| | CATGGTGGATCGGGCAGAGGCATGAGTCCTACAGGGACACCC | |
| | AGGGGGCAGACAGAGGTTAAGCGgctaagcAGCTTGGCGTAACT | |
| | AGATCT | |
| Argininosuccinate | ggccaaagcatgcatGGCCCCCTGGGTGTCCCTGTGTTTCAGAGCT | 423 |
| lyase | ATGCTGGAAACAGCATAGCAAGTTGAAATAAGGCTAGTCCGTT | |
| deficiency; | ATCAACTTGAAAAAGTGGCACCGAGTCGGTGCGGGCAGAGGC | |
| not provided | GTGAGTCCTACAGGGACACCCAGGGAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTGGA | |
| | CCATGGTGGATCGGGCAGAGGCATGAGTCCTACAGGGACACC | |
| | CAGGGGGCAGACAGAGGTTTGACCgctaagcAGCTTGGCGTAAC | |
| | TAGATCT | |
| Argininosuccinate | ggccaaagcatgcatGGCCCCCTGGGTGTCCCTGTGTTTCAGAGCT | 424 |
| lyase | ATGCTGGAAACAGCATAGCAAGTTGAAATAAGGCTAGTCCGTT | |
| deficiency; | ATCAACTTGAAAAAGTGGCACCGAGTCGGTGCGGATCGGGCA | |
| not provided | GAGGCGTGAGTCCTACAGGGACACCCAGGGAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TATGGTGGATCGGGCAGAGGCATGAGTCCTACAGGGACACCC | |
| | AGGGGGCAGACAGAGGTTACCCTgctaagcAGCTTGGCGTAACT | |
| | AGATCT | |
| Phenylketonuria; | ggccaaagcatgcatGAAAGTATTGCGCTTATTTGGTTTCAGAGCTAT | 425 |
| not | GCTGGAAACAGCATAGCAAGTTGAAATAAGGCTAGTCCGTTAT | |
| provided | CAACTTGAAAAAGTGGCACCGAGTCGGTGCGCACTGACCTCA | |
| | AATAAGCGCAATACAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTGCTACGACATTATCCA | |
| | AGACAAACATGATTGTAGCAGTGACCTCAAATAAGCGCAATAC | |
| | TTTGGCCAATGCACAAGTAgctaagcAGCTTGGCGTAACTAGATC | |
| | T | |
| Phenylketonuria; | ggccaaagcatgcatGAAAGTATTGCGCTTATTTGGTTTCAGAGCTAT | 426 |
| not | GCTGGAAACAGCATAGCAAGTTGAAATAAGGCTAGTCCGTTAT | |
| provided | CAACTTGAAAAAGTGGCACCGAGTCGGTGCTGTAGCACTGAC | |
| | CTCAAATAAGCGCAATACAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTCGACATTATCCA | |
| | AGACAAACATGATTGTAGCAGTGACCTCAAATAAGCGCAATAC | |
| | TTTGGCCAATGCAGAGTGTgctaagcAGCTTGGCGTAACTAGATC | |
| | T | |
| Phenylketonuria; | ggccaaagcatgcatGAAAGTATTGCGCTTATTTGGTTTCAGAGCTAT | 427 |
| not | GCTGGAAACAGCATAGCAAGTTGAAATAAGGCTAGTCCGTTAT | |
| provided | CAACTTGAAAAAGTGGCACCGAGTCGGTGCGATTGTAGCACT | |
| | GACCTCAAATAAGCGCAATACAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCATTATCCA | |
| | AGACAAACATGATTGTAGCAGTGACCTCAAATAAGCGCAATAC | |
| | TTTGGCCAATGCATAGAATgctaagcAGCTTGGCGTAACTAGATC | |
| | T | |
| Phenylketonuria; | ggccaaagcatgcatGAAAGTATTGCGCTTATTTGGTTTCAGAGCTAT | 428 |
| not | GCTGGAAACAGCATAGCAAGTTGAAATAAGGCTAGTCCGTTAT | |
| provided | CAACTTGAAAAAGTGGCACCGAGTCGGTGCAACATGATTGTAG | |
| | CACTGACCTCAAATAAGCGCAATACAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTTCCAA | |
| | GACAAACATGATTGTAGCAGTGACCTCAAATAAGCGCAATACT | |
| | TTGGCCAATGCATTGCAGgctaagcAGCTTGGCGTAACTAGATCT | |
| Phenylketonuria; | ggccaaagcatgcatGAGCAAGGCAGACTTACTCGGTTTCAGAGCTA | 429 |
| not | TGCTGGAAACAGCATAGCAAGTTGAAATAAGGCTAGTCCGTTA | |
| provided | TCAACTTGAAAAAGTGGCACCGAGTCGGTGCCTACCGCCAGT | |
| | AAGTCTGCCTTAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTTACCGTGCAAGACGGAAGC | |
| | AGTTTGCTGACATTGCCTACAACTACCGCGAGTAAGTCTGCCT | |
| | TGCTTGTTGAGGGGGGTGCCgctaagcAGCTTGGCGTAACTAGA | |
| | TCT | |
| Phenylketonuria; | ggccaaagcatgcatGAGCAAGGCAGACTTACTCGGTTTCAGAGCTA | 430 |
| not | TGCTGGAAACAGCATAGCAAGTTGAAATAAGGCTAGTCCGTTA | |
| provided | TCAACTTGAAAAAGTGGCACCGAGTCGGTGCCAACTACCGCC | |
| | AGTAAGTCTGCCTTAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTCGTGCAAGACGGAAG | |
| | CAGTTTGCTGACATTGCCTACAACTACCGCGAGTAAGTCTGCC | |
| | TTGCTTGTTGAGGGGGTGGGTgctaagcAGCTTGGCGTAACTAG | |
| | ATCT | |
| Phenylketonuria; | ggccaaagcatgcatGAGCAAGGCAGACTTACTCGGTTTCAGAGCTA | 431 |
| not | TGCTGGAAACAGCATAGCAAGTTGAAATAAGGCTAGTCCGTTA | |
| provided | TCAACTTGAAAAAGTGGCACCGAGTCGGTGCGCCTACAACTA | |
| | CCGCCAGTAAGTCTGCCTTAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTAAGACGGAAGC | |
| | AGTTTGCTGACATTGCCTACAACTACCGCGAGTAAGTCTGCCT | |
| | TGCTTGTTGAGGGGTACGATgctaagcAGCTTGGCGTAACTAGAT | |
| | CT | |
| Phenylketonuria; | ggccaaagcatgcatGCCAAGGAGAAAGTAAGCTGGTTTCAGAGCTA | 432 |
| not | TGCTGGAAACAGCATAGCAAGTTGAAATAAGGCTAGTCCGTTA | |
| provided | TCAACTTGAAAAAGTGGCACCGAGTCGGTGCTCACCTTACTTT | |
| | CTCCTAAAAAAAAAGCTGCCATCAGTCGGCGTGGACTGTAGAA | |
| | CACTGCCAATGCCGGTCCCAAGCCCGGATAAAAGTGGAGGGT | |
| | ACAGTCCACGCTTTTTTTTGGCACCAGTCAGGAGGCCCCCAG | |
| | AGCTAGTGGCTCACCTTTGTCACCACCTCAGCTTACTTTCTCC | |
| | TTGGCATCATTAAACCCGGCgctaagcAGCTTGGCGTAACTAGAT | |
| | CT | |
| Phenylketonuria; | ggccaaagcatgcatGCCAAGGAGAAAGTAAGCTGGTTTCAGAGCTA | 433 |
| not | TGCTGGAAACAGCATAGCAAGTTGAAATAAGGCTAGTCCGTTA | |
| provided | TCAACTTGAAAAAGTGGCACCGAGTCGGTGCCACCTCACCTTA | |
| | CTTTCTCCTAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTACCAGTCAGGAGGCCCCCAG | |
| | AGCTAGTGGCTCACCTTTGTCACCACCTCAGCTTACTTTCTCC | |
| | TTGGCATCATTAAACGACGAgctaagcAGCTTGGCGTAACTAGAT | |
| | CT | |
| Phenylketonuria; | ggccaaagcatgcatGCCAAGGAGAAAGTAAGCTGGTTTCAGAGCTA | 434 |
| not | TGCTGGAAACAGCATAGCAAGTTGAAATAAGGCTAGTCCGTTA | |
| provided | TCAACTTGAAAAAGTGGCACCGAGTCGGTGCCACCACCTCAC | |
| | CTTACTTTCTCCTAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTAGTCAGGAGGCCCCCA | |
| | GAGCTAGTGGCTCACCTTTGTCACCACCTCAGCTTACTTTCTC | |
| | CTTGGCATCATTAAATGGCGCgctaagcAGCTTGGCGTAACTAGA | |
| | TCT | |
| Phenylketonuria; | ggccaaagcatgcatGCCAAGGAGAAAGTAAGCTGGTTTCAGAGCTA | 435 |
| not | TGCTGGAAACAGCATAGCAAGTTGAAATAAGGCTAGTCCGTTA | |
| provided | TCAACTTGAAAAAGTGGCACCGAGTCGGTGCTTTGTCACCACC | |
| | TCACCTTACTTTCTCCTAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTGGAGGCCCCCAG | |
| | AGCTAGTGGCTCACCTTTGTCACCACCTCAGCTTACTTTCTCC | |
| | TTGGCATCATTAAACTTGACgctaagcAGCTTGGCGTAACTAGAT | |
| | CT | |
| Citrullinemia | ggccaaagcatgcatGGGTCACTCCCAAGAACCCGGTTTCAGAGCT | 436 |
| type | ATGCTGGAAACAGCATAGCAAGTTGAAATAAGGCTAGTCCGTT | |
| I;Citrullinemi | ATCAACTTGAAAAAGTGGCACCGAGTCGGTGCTCCACGGGTT | |
| a, mild;not | CTTGGGAGTGAAAAAAAAAGCTGCCATCAGTCGGCGTGGACT | |
| provided | GTAGAACACTGCCAATGCCGGTCCCAAGCCCGGATAAAAGTG | |
| | GAGGGTACAGTCCACGCTTTTTTTTGGAGGGTGGGATTTACCT | |
| | GATGTGCATGAGGTTCTCATCCATGCTCCGCGGGTTCTTGGG | |
| | AGTGACCGGGATGGGAAATGTGTgctaagcAGCTTGGCGTAACT | |
| | AGATCT | |
| Citrullinemia | ggccaaagcatgcatGGGTCACTCCCAAGAACCCGGTTTCAGAGCT | 437 |
| type I; | ATGCTGGAAACAGCATAGCAAGTTGAAATAAGGCTAGTCCGTT | |
| Citrullinemia, | ATCAACTTGAAAAAGTGGCACCGAGTCGGTGCATGCTCCACG | |
| mild; not | GGTTCTTGGGAGTGAAAAAAAAAGCTGCCATCAGTCGGCGTG | |
| provided | GACTGTAGAACACTGCCAATGCCGGTCCCAAGCCCGGATAAA | |
| | AGTGGAGGGTACAGTCCACGCTTTTTTTGGGTGGGATTTACCT | |
| | GATGTGCATGAGGTTCTCATCCATGCTCCGCGGGTTCTTGGG | |
| | AGTGACCGGGATGGGAAGTTGCGgctaagcAGCTTGGCGTAACT | |
| | AGATCT | |
| Citrullinemia | ggccaaagcatgcatGGGTCACTCCCAAGAACCCGGTTTCAGAGCT | 438 |
| type I; | ATGCTGGAAACAGCATAGCAAGTTGAAATAAGGCTAGTCCGTT | |
| Citrullinemia, | ATCAACTTGAAAAAGTGGCACCGAGTCGGTGCTCCATGCTCCA | |
| mild; not | CGGGTTCTTGGGAGTGAAAAAAAAAGCTGCCATCAGTCGGCG | |
| provided | TGGACTGTAGAACACTGCCAATGCCGGTCCCAAGCCCGGATA | |
| | AAAGTGGAGGGTACAGTCCACGCTTTTTTTTGGGATTTACCTG | |
| | ATGTGCATGAGGTTCTCATCCATGCTCCGCGGGTTCTTGGGA | |
| | GTGACCGGGATGGGAATATTCGgctaagcAGCTTGGCGTAACTA | |
| | GATCT | |
| Citrullinemia | ggccaaagcatgcatGGGTCACTCCCAAGAACCCGGTTTCAGAGCT | 439 |
| type I; | ATGCTGGAAACAGCATAGCAAGTTGAAATAAGGCTAGTCCGTT | |
| Citrullinemia, | ATCAACTTGAAAAAGTGGCACCGAGTCGGTGCTCTCATCCATG | |
| mild; not | CTCCACGGGTTCTTGGGAGTGAAAAAAAAAGCTGCCATCAGTC | |
| provided | GGCGTGGACTGTAGAACACTGCCAATGCCGGTCCCAAGCCCG | |
| | GATAAAAGTGGAGGGTACAGTCCACGCTTTTTTTTTTACCTGAT | |
| | GTGCATGAGGTTCTCATCCATGCTCCGCGGGTTCTTGGGAGT | |
| | GACCGGGATGGGAACTCTCGgctaagcAGCTTGGCGTAACTAGA | |
| | TCT | |
| Citrullinemia | ggccaaagcatgcatGGGTTCTCATCCATGCTCCGGTTTCAGAGCTA | 440 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCCCGTGGAGCAT | |
| mild; not | GGATGAGAAAAAAAAAAGCTGCCATCAGTCGGCGTGGACTGT | |
| provided | AGAACACTGCCAATGCCGGTCCCAAGCCCGGATAAAAGTGGA | |
| | GGGTACAGTCCACGCTTTTTTTCTTTCCTGCAGCAACACGGGA | |
| | TTCCCATCCCGGTCACTCCCAAGAACCCGCGGAGCATGGATG | |
| | AGAACCTCATGCACATGCTAGTgctaagcAGCTTGGCGTAACTAG | |
| | ATCT | |
| Citrullinemia | ggccaaagcatgcatGGGTTCTCATCCATGCTCCGGTTTCAGAGCTA | 441 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCGAACCCGTGGA | |
| mild; not | GCATGGATGAGAAAAAAAAAAGCTGCCATCAGTCGGCGTGGA | |
| provided | CTGTAGAACACTGCCAATGCCGGTCCCAAGCCCGGATAAAAG | |
| | TGGAGGGTACAGTCCACGCTTTTTTTCCTGCAGCAACACGGG | |
| | ATTCCCATCCCGGTCACTCCCAAGAACCCGCGGAGCATGGAT | |
| | GAGAACCTCATGCACATAACCGTgctaagcAGCTTGGCGTAACTA | |
| | GATCT | |
| Citrullinemia | ggccaaagcatgcatGGGTTCTCATCCATGCTCCGGTTTCAGAGCTA | 442 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCCAAGAACCCGT | |
| mild; not | GGAGCATGGATGAGAAAAAAAAAAGCTGCCATCAGTCGGCGT | |
| provided | GGACTGTAGAACACTGCCAATGCCGGTCCCAAGCCCGGATAA | |
| | AAGTGGAGGGTACAGTCCACGCTTTTTTTGCAGCAACACGGG | |
| | ATTCCCATCCCGGTCACTCCCAAGAACCCGCGGAGCATGGAT | |
| | GAGAACCTCATGCACATTGGTAGgctaagcAGCTTGGCGTAACTA | |
| | GATCT | |
| Citrullinemia | ggccaaagcatgcatGGGTTCTCATCCATGCTCCGGTTTCAGAGCTA | 443 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCACTCCCAAGAA | |
| mild; not | CCCGTGGAGCATGGATGAGAAAAAAAAAAGCTGCCATCAGTC | |
| provided | GGCGTGGACTGTAGAACACTGCCAATGCCGGTCCCAAGCCCG | |
| | GATAAAAGTGGAGGGTACAGTCCACGCTTTTTTTAACACGGGA | |
| | TTCCCATCCCGGTCACTCCCAAGAACCCGCGGAGCATGGATG | |
| | AGAACCTCATGCACATGCTTTTgctaagcAGCTTGGCGTAACTAG | |
| | ATCT | |
| Citrullinemia | ggccaaagcatgcatGGTTCTCATCCATGCTCCGCGTTTCAGAGCTA | 444 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCCCGTGGAGCAT | |
| mild; not | GGATGAGAAAAAAAAAGCTGCCATCAGTCGGCGTGGACTGTA | |
| provided | GAACACTGCCAATGCCGGTCCCAAGCCCGGATAAAAGTGGAG | |
| | GGTACAGTCCACGCTTTTTTTCTCTTTCCTGCAGCAACACGGG | |
| | ATTCCCATCCCGGTCACTCCCAAGAACCCGCGGAGCATGGAT | |
| | GAGAACCTCATGCACACCGTACgctaagcAGCTTGGCGTAACTA | |
| | GATCT | |
| Citrullinemia | ggccaaagcatgcatGGTTCTCATCCATGCTCCGCGTTTCAGAGCTA | 445 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCGAACCCGTGGA | |
| mild; not | GCATGGATGAGAAAAAAAAAGCTGCCATCAGTCGGCGTGGAC | |
| provided | TGTAGAACACTGCCAATGCCGGTCCCAAGCCCGGATAAAAGT | |
| | GGAGGGTACAGTCCACGCTTTTTTTTTCCTGCAGCAACACGGG | |
| | ATTCCCATCCCGGTCACTCCCAAGAACCCGCGGAGCATGGAT | |
| | GAGAACCTCATGCACACTAGTTgctaagcAGCTTGGCGTAACTAG | |
| | ATCT | |
| Citrullinemia | ggccaaagcatgcatGGTTCTCATCCATGCTCCGCGTTTCAGAGCTA | 446 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCCAAGAACCCGT | |
| mild; not | GGAGCATGGATGAGAAAAAAAAAGCTGCCATCAGTCGGCGTG | |
| provided | GACTGTAGAACACTGCCAATGCCGGTCCCAAGCCCGGATAAA | |
| | AGTGGAGGGTACAGTCCACGCTTTTTTTCTGCAGCAACACGG | |
| | GATTCCCATCCCGGTCACTCCCAAGAACCCGCGGAGCATGGA | |
| | TGAGAACCTCATGCACAATACACgctaagcAGCTTGGCGTAACTA | |
| | GATCT | |
| Citrullinemia | ggccaaagcatgcatGGTTCTCATCCATGCTCCGCGTTTCAGAGCTA | 447 |
| type I; | TGCTGGAAACAGCATAGCAAGTTGAAATAAGGCTAGTCCGTTA | |
| Citrullinemia, | TCAACTTGAAAAAGTGGCACCGAGTCGGTGCACTCCCAAGAA | |
| mild; not | CCCGTGGAGCATGGATGAGAAAAAAAAAGCTGCCATCAGTCG | |
| provided | GCGTGGACTGTAGAACACTGCCAATGCCGGTCCCAAGCCCGG | |
| | ATAAAAGTGGAGGGTACAGTCCACGCTTTTTTTGCAACACGGG | |
| | ATTCCCATCCCGGTCACTCCCAAGAACCCGCGGAGCATGGAT | |
| | GAGAACCTCATGCACATGCTCAgctaagcAGCTTGGCGTAACTAG | |
| | ATCT | |
| Ornithine | ggccaaagcatgcatGTTAAATTCTTCCTCCTTTAGTTTCAGAGCTAT | 448 |
| carbamoyl- | GCTGGAAACAGCATAGCAAGTTGAAATAAGGCTAGTCCGTTAT | |
| transferase | CAACTTGAAAAAGTGGCACCGAGTCGGTGCTCCGGCTCATAA | |
| deficiency; | CCCTAAAGGAGGAAGAATTAAAAAAAAAGCTGCCATCAGTCGG | |
| not provided | CGTGGACTGTAGAACACTGCCAATGCCGGTCCCAAGCCCGGA | |
| | TAAAAGTGGAGGGTACAGTCCACGCTTTTTTTCTGCCAACTTG | |
| | GTTACACTAGCATCCAGCTCATAACCCTAAAGGAGGAAGAATT | |
| | TAAATCTTATTTACGCGGTgctaagcAGCTTGGCGTAACTAGATC | |
| | T | |
| Ornithine | ggccaaagcatgcatGTTAAATTCTTCCTCCTTTAGTTTCAGAGCTAT | 449 |
| carbamoyl- | GCTGGAAACAGCATAGCAAGTTGAAATAAGGCTAGTCCGTTAT | |
| transferase | CAACTTGAAAAAGTGGCACCGAGTCGGTGCGTTACACTAGCAT | |
| deficiency; | CCGGCTCATAACCCTAAAGGAGGAAGAATTAAAAAAAAAGCTG | |
| not provided | CCATCAGTCGGCGTGGACTGTAGAACACTGCCAATGCCGGTC | |
| | CCAAGCCCGGATAAAAGTGGAGGGTACAGTCCACGCTTTTTTT | |
| | TTACACTAGCATCCAGCTCATAACCCTAAAGGAGGAAGAATTT | |
| | AAATCTTATTTAAATGTGgctaagcAGCTTGGCGTAACTAGATCT | |
| Ornithine | ggccaaagcatgcatGCTCCTTTAGGGTTATGAGCGTTTCAGAGCTA | 450 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAGCATCCGGCT | |
| deficiency; | CATAACCCTAAAGAAAAAAAAAGCTGCCATCAGTCGGCGTGGA | |
| not provided | CTGTAGAACACTGCCAATGCCGGTCCCAAGCCCGGATAAAAG | |
| | TGGAGGGTACAGTCCACGCTTTTTTTCCTCTTTGGCATACTGC | |
| | TCTGCCAACTTGGTTACACTAGCATCCAGCTCATAACCCTAAA | |
| | GGAGGAAGAATTTACAAATCgctaagcAGCTTGGCGTAACTAGAT | |
| | CT | |
| Ornithine | ggccaaagcatgcatGCTCCTTTAGGGTTATGAGCGTTTCAGAGCTA | 451 |
| carbamoyl- | TGCTGGAAACAGCATAGCAAGTTGAAATAAGGCTAGTCCGTTA | |
| transferase | TCAACTTGAAAAAGTGGCACCGAGTCGGTGCACTAGCATCCG | |
| deficiency; | GCTCATAACCCTAAAGAAAAAAAAAGCTGCCATCAGTCGGCGT | |
| not provided | GGACTGTAGAACACTGCCAATGCCGGTCCCAAGCCCGGATAA | |
| | AAGTGGAGGGTACAGTCCACGCTTTTTTTCTTTGGCATACTGC | |
| | TCTGCCAACTTGGTTACACTAGCATCCAGCTCATAACCCTAAA | |
| | GGAGGAAGAATTTACGTTAAgctaagcAGCTTGGCGTAACTAGAT | |
| | CT | |
| Ornithine | ggccaaagcatgcatGCTCCTTTAGGGTTATGAGCGTTTCAGAGCTA | 452 |
| carbamoyl- | TGCTGGAAACAGCATAGCAAGTTGAAATAAGGCTAGTCCGTTA | |
| transferase | TCAACTTGAAAAAGTGGCACCGAGTCGGTGCGTTACACTAGCA | |
| deficiency; | TCCGGCTCATAACCCTAAAGAAAAAAAAAGCTGCCATCAGTCG | |
| not provided | GCGTGGACTGTAGAACACTGCCAATGCCGGTCCCAAGCCCGG | |
| | ATAAAAGTGGAGGGTACAGTCCACGCTTTTTTTGCATACTGCT | |
| | CTGCCAACTTGGTTACACTAGCATCCAGCTCATAACCCTAAAG | |
| | GAGGAAGAATTTATCAGCGgctaagcAGCTTGGCGTAACTAGATC | |
| | T | |
| Argininosuccinate | ggccaaagcatgcatGAGCAGCCGCTCAGAGTCTCGTTTCAGAGCT | 453 |
| lyase | ATGCTGGAAACAGCATAGCAAGTTGAAATAAGGCTAGTCCGTT | |
| deficiency; | ATCAACTTGAAAAAGTGGCACCGAGTCGGTGCACCCGAGACT | |
| not provided | CTGAGCGGCTAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTCTGACCCTCCTGCCCCTGG | |
| | CTTCCCACAGCCACGCCGTGGCACTGACCTGAGACTCTGAGC | |
| | GGCTGCTGGAGGTGCGGAGTTTCgctaagcAGCTTGGCGTAACT | |
| | AGATCT | |
| Argininosuccinate | ggccaaagcatgcatGAGCAGCCGCTCAGAGTCTCGTTTCAGAGCT | 454 |
| lyase | ATGCTGGAAACAGCATAGCAAGTTGAAATAAGGCTAGTCCGTT | |
| deficiency; | ATCAACTTGAAAAAGTGGCACCGAGTCGGTGCACTGACCCGA | |
| not provided | GACTCTGAGCGGCTAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTCCCTCCTGCCCCTGG | |
| | CTTCCCACAGCCACGCCGTGGCACTGACCTGAGACTCTGAGC | |
| | GGCTGCTGGAGGTGCGGTGCGAAgctaagcAGCTTGGCGTAACT | |
| | AGATCT | |
| Argininosuccinate | ggccaaagcatgcatGAGCAGCCGCTCAGAGTCTCGTTTCAGAGCT | 455 |
| lyase | ATGCTGGAAACAGCATAGCAAGTTGAAATAAGGCTAGTCCGTT | |
| deficiency; | ATCAACTTGAAAAAGTGGCACCGAGTCGGTGCGGCACTGACC | |
| not provided | CGAGACTCTGAGCGGCTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTTCCTGCCCCTGG | |
| | CTTCCCACAGCCACGCCGTGGCACTGACCTGAGACTCTGAGC | |
| | GGCTGCTGGAGGTGCGGAATGAAgctaagcAGCTTGGCGTAACT | |
| | AGATCT | |
| Argininosuccinate | ggccaaagcatgcatGAGCAGCCGCTCAGAGTCTCGTTTCAGAGCT | 456 |
| lyase | ATGCTGGAAACAGCATAGCAAGTTGAAATAAGGCTAGTCCGTT | |
| deficiency; | ATCAACTTGAAAAAGTGGCACCGAGTCGGTGCGCCGTGGCAC | |
| not provided | TGACCCGAGACTCTGAGCGGCTAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTCCCCTGG | |
| | CTTCCCACAGCCACGCCGTGGCACTGACCTGAGACTCTGAGC | |
| | GGCTGCTGGAGGTGCGGTTGGAGgctaagcAGCTTGGCGTAACT | |
| | AGATCT | |
| Phenylketonuria; | ggccaaagcatgcatGTCCTGTGTACCGTGCAAGAGTTTCAGAGCTA | 457 |
| not | TGCTGGAAACAGCATAGCAAGTTGAAATAAGGCTAGTCCGTTA | |
| provided | TCAACTTGAAAAAGTGGCACCGAGTCGGTGCTCCGTCTTGCA | |
| | CGGTACACAAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTGGCAGACTTACTGGCGGTAG | |
| | TTGTAGGCAATGTCAGCAAACTGCTTCCATCTTGCACGGTACA | |
| | CAGGATCTTTAAAACACGGCTgctaagcAGCTTGGCGTAACTAGA | |
| | TCT | |
| Phenylketonuria; | ggccaaagcatgcatGTCCTGTGTACCGTGCAAGAGTTTCAGAGCTA | 458 |
| not | TGCTGGAAACAGCATAGCAAGTTGAAATAAGGCTAGTCCGTTA | |
| provided | TCAACTTGAAAAAGTGGCACCGAGTCGGTGCTGCTTCCGTCTT | |
| | GCACGGTACACAAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTGACTTACTGGCGGTAGT | |
| | TGTAGGCAATGTCAGCAAACTGCTTCCATCTTGCACGGTACAC | |
| | AGGATCTTTAAAACTCCGCAgctaagcAGCTTGGCGTAACTAGAT | |
| | CT | |
| Phenylketonuria; | ggccaaagcatgcatGTCCTGTGTACCGTGCAAGAGTTTCAGAGCTA | 459 |
| not | TGCTGGAAACAGCATAGCAAGTTGAAATAAGGCTAGTCCGTTA | |
| provided | TCAACTTGAAAAAGTGGCACCGAGTCGGTGCAACTGCTTCCGT | |
| | CTTGCACGGTACACAAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTTTACTGGCGGTAGT | |
| | TGTAGGCAATGTCAGCAAACTGCTTCCATCTTGCACGGTACAC | |
| | AGGATCTTTAAAACCTTTTCgctaagcAGCTTGGCGTAACTAGAT | |
| | CT | |
| Phenylketonuria; | ggccaaagcatgcatGTCCTGTGTACCGTGCAAGAGTTTCAGAGCTA | 460 |
| not | TGCTGGAAACAGCATAGCAAGTTGAAATAAGGCTAGTCCGTTA | |
| provided | TCAACTTGAAAAAGTGGCACCGAGTCGGTGCCAGCAAACTGC | |
| | TTCCGTCTTGCACGGTACACAAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTGGCGGTAGT | |
| | TGTAGGCAATGTCAGCAAACTGCTTCCATCTTGCACGGTACAC | |
| | AGGATCTTTAAAACGTGCGCgctaagcAGCTTGGCGTAACTAGAT | |
| | CT | |
| Hyperphenyl | ggccaaagcatgcatGGGGCAGCCCATCCCTCGAAGTTTCAGAGCT | 461 |
| alaninemia, | ATGCTGGAAACAGCATAGCAAGTTGAAATAAGGCTAGTCCGTT | |
| non- | ATCAACTTGAAAAAGTGGCACCGAGTCGGTGCCCACTCGAGG | |
| pku; | GATGGGCTGAAAAAAAAAGCTGCCATCAGTCGGCGTGGACTG | |
| Phenylketonuria; | TAGAACACTGCCAATGCCGGTCCCAAGCCCGGATAAAAGTGG | |
| not provided | AGGGTACAGTCCACGCTTTTTTTAGTCTTGAACACTGTGCCCC | |
| | ATGTTTTCTTTTCTTCCTCCATGTATTCCATTCGAGGGATGGGC | |
| | TGCCCACTAGAATACCGTCTAgctaagcAGCTTGGCGTAACTAGA | |
| | TCT | |
| Hyperphenyl | ggccaaagcatgcatGGGGCAGCCCATCCCTCGAAGTTTCAGAGCT | 462 |
| alaninemia, | ATGCTGGAAACAGCATAGCAAGTTGAAATAAGGCTAGTCCGTT | |
| non- | ATCAACTTGAAAAAGTGGCACCGAGTCGGTGCTATTCCACTCG | |
| pku; | AGGGATGGGCTGAAAAAAAAAGCTGCCATCAGTCGGCGTGGA | |
| Phenylketonuria; | CTGTAGAACACTGCCAATGCCGGTCCCAAGCCCGGATAAAAG | |
| not provided | TGGAGGGTACAGTCCACGCTTTTTTTTTGAACACTGTGCCCCA | |
| | TGTTTTCTTTTCTTCCTCCATGTATTCCATTCGAGGGATGGGCT | |
| | GCCCACTAGAATACTTCTCAgctaagcAGCTTGGCGTAACTAGAT | |
| | CT | |
| Hyperphenyl | ggccaaagcatgcatGGGGCAGCCCATCCCTCGAAGTTTCAGAGCT | 463 |
| alaninemia, | ATGCTGGAAACAGCATAGCAAGTTGAAATAAGGCTAGTCCGTT | |
| non- | ATCAACTTGAAAAAGTGGCACCGAGTCGGTGCATGTATTCCAC | |
| pku; | TCGAGGGATGGGCTGAAAAAAAAAGCTGCCATCAGTCGGCGT | |
| Phenylketonuria; | GGACTGTAGAACACTGCCAATGCCGGTCCCAAGCCCGGATAA | |
| not provided | AAGTGGAGGGTACAGTCCACGCTTTTTTTAACACTGTGCCCCA | |
| | TGTTTTCTTTTCTTCCTCCATGTATTCCATTCGAGGGATGGGCT | |
| | GCCCACTAGAATACCAACGCgctaagcAGCTTGGCGTAACTAGAT | |
| | CT | |
| Hyperphenyl | ggccaaagcatgcatGGGGCAGCCCATCCCTCGAAGTTTCAGAGCT | 464 |
| alaninemia, | ATGCTGGAAACAGCATAGCAAGTTGAAATAAGGCTAGTCCGTT | |
| non- | ATCAACTTGAAAAAGTGGCACCGAGTCGGTGCCCTCCATGTAT | |
| pku; | TCCACTCGAGGGATGGGCTGAAAAAAAAAGCTGCCATCAGTC | |
| Phenylketonuria; | GGCGTGGACTGTAGAACACTGCCAATGCCGGTCCCAAGCCCG | |
| not provided | GATAAAAGTGGAGGGTACAGTCCACGCTTTTTTTTGTGCCCCA | |
| | TGTTTTCTTTTCTTCCTCCATGTATTCCATTCGAGGGATGGGCT | |
| | GCCCACTAGAATACATGAGTgctaagcAGCTTGGCGTAACTAGAT | |
| | CT | |
| Hyperphenyl | ggccaaagcatgcatGTTCCTTACCTGGGAAAACTGTTTCAGAGCTA | 465 |
| alaninemia, | TGCTGGAAACAGCATAGCAAGTTGAAATAAGGCTAGTCCGTTA | |
| non- | TCAACTTGAAAAAGTGGCACCGAGTCGGTGCATCGCAGCTTT | |
| pku; | GCCCAGTTTTCCCAGGTAAGAAAAAAAAAGCTGCCATCAGTCG | |
| Phenylketonuria; | GCGTGGACTGTAGAACACTGCCAATGCCGGTCCCAAGCCCGG | |
| not provided | ATAAAAGTGGAGGGTACAGTCCACGCTTTTTTTGGGACATGTG | |
| | CCCTTGTTTTCAGATCACAGCTTTGCCCAGTTTTCCCAGGTAA | |
| | GGAATGGATTTTTTGCAACAgctaagcAGCTTGGCGTAACTAGAT | |
| | CT | |
| Hyperphenyl | ggccaaagcatgcatGTTCCTTACCTGGGAAAACTGTTTCAGAGCTA | 466 |
| alaninemia, | TGCTGGAAACAGCATAGCAAGTTGAAATAAGGCTAGTCCGTTA | |
| non- | TCAACTTGAAAAAGTGGCACCGAGTCGGTGCTTTTCAGATCGC | |
| pku; | AGCTTTGCCCAGTTTTCCCAGGTAAGAAAAAAAAAGCTGCCAT | |
| Phenylketonuria; | CAGTCGGCGTGGACTGTAGAACACTGCCAATGCCGGTCCCAA | |
| not provided | GCCCGGATAAAAGTGGAGGGTACAGTCCACGCTTTTTTTGTGC | |
| | CCTTGTTTTCAGATCACAGCTTTGCCCAGTTTTCCCAGGTAAG | |
| | GAATGGATTTTTTTGACATgctaagcAGCTTGGCGTAACTAGATCT | |
| Hyperphenyl | ggccaaagcatgcatGTTCCTTACCTGGGAAAACTGTTTCAGAGCTA | 467 |
| alaninemia, | TGCTGGAAACAGCATAGCAAGTTGAAATAAGGCTAGTCCGTTA | |
| non- | TCAACTTGAAAAAGTGGCACCGAGTCGGTGCCCTTGTTTTCAG | |
| pku; | ATCGCAGCTTTGCCCAGTTTTCCCAGGTAAGAAAAAAAAAGCT | |
| Phenylketonuria; | GCCATCAGTCGGCGTGGACTGTAGAACACTGCCAATGCCGGT | |
| not provided | CCCAAGCCCGGATAAAAGTGGAGGGTACAGTCCACGCTTTTTT | |
| | TCTTGTTTTCAGATCACAGCTTTGCCCAGTTTTCCCAGGTAAG | |
| | GAATGGATTTTTTTCGACCgctaagcAGCTTGGCGTAACTAGATC | |
| | T | |
| Hyperphenyl | ggccaaagcatgcatGTTGTACTCATAGCAAGCATGTTTCAGAGCTA | 468 |
| alaninemia, | TGCTGGAAACAGCATAGCAAGTTGAAATAAGGCTAGTCCGTTA | |
| non- | TCAACTTGAAAAAGTGGCACCGAGTCGGTGCACCCATGCTTG | |
| pku;Phenylk | CTATGAGTAAAAAAAAAAGCTGCCATCAGTCGGCGTGGACTGT | |
| etonuria;not | AGAACACTGCCAATGCCGGTCCCAAGCCCGGATAAAAGTGGA | |
| provided | GGGTACAGTCCACGCTTTTTTTAAAACATGGGGCACAGTGTTC | |
| | AAGACTCTGAAGTCCTTGTATAAAACCTATGCTTGCTATGAGTA | |
| | CAATCACATTTTTGAGGTAgctaagcAGCTTGGCGTAACTAGATC | |
| | T | |
| Hyperphenyl | ggccaaagcatgcatGTTGTACTCATAGCAAGCATGTTTCAGAGCTA | 469 |
| alaninemia, | TGCTGGAAACAGCATAGCAAGTTGAAATAAGGCTAGTCCGTTA | |
| non- | TCAACTTGAAAAAGTGGCACCGAGTCGGTGCTAAAACCCATGC | |
| pku; | TTGCTATGAGTAAAAAAAAAAGCTGCCATCAGTCGGCGTGGAC | |
| Phenylketonuria; | TGTAGAACACTGCCAATGCCGGTCCCAAGCCCGGATAAAAGT | |
| not provided | GGAGGGTACAGTCCACGCTTTTTTTCATGGGGCACAGTGTTCA | |
| | AGACTCTGAAGTCCTTGTATAAAACCTATGCTTGCTATGAGTAC | |
| | AATCACATTTTTCCCTTAgctaagcAGCTTGGCGTAACTAGATCT | |
| Hyperphenyl | ggccaaagcatgcatGTTGTACTCATAGCAAGCATGTTTCAGAGCTA | 470 |
| alaninemia, | TGCTGGAAACAGCATAGCAAGTTGAAATAAGGCTAGTCCGTTA | |
| non- | TCAACTTGAAAAAGTGGCACCGAGTCGGTGCGTATAAAACCCA | |
| pku; | TGCTTGCTATGAGTAAAAAAAAAAGCTGCCATCAGTCGGCGTG | |
| Phenylketonuria; | GACTGTAGAACACTGCCAATGCCGGTCCCAAGCCCGGATAAA | |
| not provided | AGTGGAGGGTACAGTCCACGCTTTTTTTGGGGCACAGTGTTCA | |
| | AGACTCTGAAGTCCTTGTATAAAACCTATGCTTGCTATGAGTAC | |
| | AATCACATTTTTTCTGGGgctaagcAGCTTGGCGTAACTAGATCT | |
| Hyperphenyl | ggccaaagcatgcatGTTGTACTCATAGCAAGCATGTTTCAGAGCTA | 471 |
| alaninemia, | TGCTGGAAACAGCATAGCAAGTTGAAATAAGGCTAGTCCGTTA | |
| non- | TCAACTTGAAAAAGTGGCACCGAGTCGGTGCTCCTTGTATAAA | |
| pku; | ACCCATGCTTGCTATGAGTAAAAAAAAAAGCTGCCATCAGTCG | |
| Phenylketonuria; | GCGTGGACTGTAGAACACTGCCAATGCCGGTCCCAAGCCCGG | |
| not provided | ATAAAAGTGGAGGGTACAGTCCACGCTTTTTTTACAGTGTTCA | |
| | AGACTCTGAAGTCCTTGTATAAAACCTATGCTTGCTATGAGTAC | |
| | AATCACATTTTTCCGTTTgctaagcAGCTTGGCGTAACTAGATCT | |
| Phenylketonuria; | ggccaaagcatgcatGATGGCTTTACTTTATTTTCGTTTCAGAGCTAT | 472 |
| not | GCTGGAAACAGCATAGCAAGTTGAAATAAGGCTAGTCCGTTAT | |
| provided | CAACTTGAAAAAGTGGCACCGAGTCGGTGCGTGCCCTCCAGA | |
| | AAATAAAGTAAAGCAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTTTTTCTTTGTAGGTGAA | |
| | ATTGGAATCCTTTGCAGTGACCTCCAGAAAATAAAGTAAAGCC | |
| | ATGGACAGAATGGGTACTgctaagcAGCTTGGCGTAACTAGATCT | |
| Phenylketonuria; | ggccaaagcatgcatGATGGCTTTACTTTATTTTCGTTTCAGAGCTAT | 473 |
| not | GCTGGAAACAGCATAGCAAGTTGAAATAAGGCTAGTCCGTTAT | |
| provided | CAACTTGAAAAAGTGGCACCGAGTCGGTGCTGCAGTGCCCTC | |
| | CAGAAAATAAAGTAAAGCAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTCTTTGTAGGTGA | |
| | AATTGGAATCCTTTGCAGTGACCTCCAGAAAATAAAGTAAAGC | |
| | CATGGACAGAATGGATCTGgctaagcAGCTTGGCGTAACTAGATC | |
| | T | |
| Phenylketonuria; | ggccaaagcatgcatGATGGCTTTACTTTATTTTCGTTTCAGAGCTAT | 474 |
| not | GCTGGAAACAGCATAGCAAGTTGAAATAAGGCTAGTCCGTTAT | |
| provided | CAACTTGAAAAAGTGGCACCGAGTCGGTGCCTTTGCAGTGCC | |
| | CTCCAGAAAATAAAGTAAAGCAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTTGTAGGTGA | |
| | AATTGGAATCCTTTGCAGTGACCTCCAGAAAATAAAGTAAAGC | |
| | CATGGACAGAATGGATCGGgctaagcAGCTTGGCGTAACTAGAT | |
| | CT | |
| Phenylketonuria; | ggccaaagcatgcatGATGGCTTTACTTTATTTTCGTTTCAGAGCTAT | 475 |
| not | GCTGGAAACAGCATAGCAAGTTGAAATAAGGCTAGTCCGTTAT | |
| provided | CAACTTGAAAAAGTGGCACCGAGTCGGTGCGAATCCTTTGCA | |
| | GTGCCCTCCAGAAAATAAAGTAAAGCAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTGTGA | |
| | AATTGGAATCCTTTGCAGTGACCTCCAGAAAATAAAGTAAAGC | |
| | CATGGACAGAATGTCTGCCgctaagcAGCTTGGCGTAACTAGATC | |
| | T | |
| Phenylketonuria; | ggccaaagcatgcatGGCTTTACTTTATTTTCTGGGTTTCAGAGCTAT | 476 |
| not | GCTGGAAACAGCATAGCAAGTTGAAATAAGGCTAGTCCGTTAT | |
| provided | CAACTTGAAAAAGTGGCACCGAGTCGGTGCGTGCCCTCCAGA | |
| | AAATAAAGTAAAAAAAAAAAGCTGCCATCAGTCGGCGTGGACT | |
| | GTAGAACACTGCCAATGCCGGTCCCAAGCCCGGATAAAAGTG | |
| | GAGGGTACAGTCCACGCTTTTTTTTGGTGTTTTTCTTTGTAGGT | |
| | GAAATTGGAATCCTTTGCAGTGACCTCCAGAAAATAAAGTAAA | |
| | GCCATGGACAGACCAAAAgctaagcAGCTTGGCGTAACTAGATCT | |
| Phenylketonuria; | ggccaaagcatgcatGGCTTTACTTTATTTTCTGGGTTTCAGAGCTAT | 477 |
| not | GCTGGAAACAGCATAGCAAGTTGAAATAAGGCTAGTCCGTTAT | |
| provided | CAACTTGAAAAAGTGGCACCGAGTCGGTGCTGCAGTGCCCTC | |
| | CAGAAAATAAAGTAAAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGTTTTTCTTTGTAGGT | |
| | GAAATTGGAATCCTTTGCAGTGACCTCCAGAAAATAAAGTAAA | |
| | GCCATGGACAGACCCAATgctaagcAGCTTGGCGTAACTAGATCT | |
| Phenylketonuria; | ggccaaagcatgcatGGCTTTACTTTATTTTCTGGGTTTCAGAGCTAT | 478 |
| not | GCTGGAAACAGCATAGCAAGTTGAAATAAGGCTAGTCCGTTAT | |
| provided | CAACTTGAAAAAGTGGCACCGAGTCGGTGCGAATCCTTTGCA | |
| | GTGCCCTCCAGAAAATAAAGTAAAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTTTGTAGG | |
| | TGAAATTGGAATCCTTTGCAGTGACCTCCAGAAAATAAAGTAA | |
| | AGCCATGGACAGATCACATgctaagcAGCTTGGCGTAACTAGATC | |
| | T | |
| Phenylketonuria; | ggccaaagcatgcatGTCGGGGGTATACATGGGCTGTTTCAGAGCT | 479 |
| not | ATGCTGGAAACAGCATAGCAAGTTGAAATAAGGCTAGTCCGTT | |
| provided | ATCAACTTGAAAAAGTGGCACCGAGTCGGTGCTACATCAGACA | |
| | TGGATCCAAGCCCATGTATACCCCAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTTTCCAC | |
| | TGCACACAGTACATCAAACATGGATCCAAGCCCATGTATACCC | |
| | CCGAACCGTGAGTAGGTTGCgctaagcAGCTTGGCGTAACTAGA | |
| | TCT | |
| Phenylketonuria; | ggccaaagcatgcatGACTCATCTTTCTTTAAACGGTTTCAGAGCTAT | 480 |
| not | GCTGGAAACAGCATAGCAAGTTGAAATAAGGCTAGTCCGTTAT | |
| provided | CAACTTGAAAAAGTGGCACCGAGTCGGTGCCCTTCTCGTTTAA | |
| | AGAAAGATGAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTTCTTCTAGGAGAATGATGTAA | |
| | ACCTGACCCACATTGAATCTAGACCTCGTTTAAAGAAAGATGA | |
| | GTATGAATTTTTAGAGCCgctaagcAGCTTGGCGTAACTAGATCT | |
| Phenylketonuria; | ggccaaagcatgcatGACTCATCTTTCTTTAAACGGTTTCAGAGCTAT | 481 |
| not | GCTGGAAACAGCATAGCAAGTTGAAATAAGGCTAGTCCGTTAT | |
| provided | CAACTTGAAAAAGTGGCACCGAGTCGGTGCTAGACCTTCTCGT | |
| | TTAAAGAAAGATGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTCTAGGAGAATGATGTAA | |
| | ACCTGACCCACATTGAATCTAGACCTCGTTTAAAGAAAGATGA | |
| | GTATGAATTTTTTATTCAgctaagcAGCTTGGCGTAACTAGATCT | |
| Phenylketonuria; | ggccaaagcatgcatGACTCATCTTTCTTTAAACGGTTTCAGAGCTAT | 482 |
| not | GCTGGAAACAGCATAGCAAGTTGAAATAAGGCTAGTCCGTTAT | |
| provided | CAACTTGAAAAAGTGGCACCGAGTCGGTGCATCTAGACCTTCT | |
| | CGTTTAAAGAAAGATGAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTGGAGAATGATGTAA | |
| | ACCTGACCCACATTGAATCTAGACCTCGTTTAAAGAAAGATGA | |
| | GTATGAATTTTTCACGACgctaagcAGCTTGGCGTAACTAGATCT | |
| Phenylketonuria; | ggccaaagcatgcatGACTCATCTTTCTTTAAACGGTTTCAGAGCTAT | 483 |
| not | GCTGGAAACAGCATAGCAAGTTGAAATAAGGCTAGTCCGTTAT | |
| provided | CAACTTGAAAAAGTGGCACCGAGTCGGTGCATTGAATCTAGAC | |
| | CTTCTCGTTTAAAGAAAGATGAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTATGATGTAA | |
| | ACCTGACCCACATTGAATCTAGACCTCGTTTAAAGAAAGATGA | |
| | GTATGAATTTTTTCAAAAgctaagcAGCTTGGCGTAACTAGATCT | |
| Phenylketonuria; | ggccaaagcatgcatGACAAGCAAGGCAGACTTACGTTTCAGAGCTA | 484 |
| not | TGCTGGAAACAGCATAGCAAGTTGAAATAAGGCTAGTCCGTTA | |
| provided | TCAACTTGAAAAAGTGGCACCGAGTCGGTGCACTACCGCCAG | |
| | TAAGTCTGCCTTGCTAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTGCAAGACGGAAGCA | |
| | GTTTGCTGACATTGCCTACAACTCCGCCAGTAAGTCTGCCTTG | |
| | CTTGTTGAGGGGAAGTTGGTTgctaagcAGCTTGGCGTAACTAGA | |
| | TCT | |
| Phenylketonuria; | ggccaaagcatgcatGACAAGCAAGGCAGACTTACGTTTCAGAGCTA | 485 |
| not | TGCTGGAAACAGCATAGCAAGTTGAAATAAGGCTAGTCCGTTA | |
| provided | TCAACTTGAAAAAGTGGCACCGAGTCGGTGCTACAACTACCG | |
| | CCAGTAAGTCTGCCTTGCTAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTGACGGAAGCAG | |
| | TTTGCTGACATTGCCTACAACTCCGCCAGTAAGTCTGCCTTGC | |
| | TTGTTGAGGGGAAGTTCGGTgctaagcAGCTTGGCGTAACTAGAT | |
| | CT | |
| Phenylketonuria; | ggccaaagcatgcatGAGCAAGGCAGACTTACTGGGTTTCAGAGCTA | 486 |
| not | TGCTGGAAACAGCATAGCAAGTTGAAATAAGGCTAGTCCGTTA | |
| provided | TCAACTTGAAAAAGTGGCACCGAGTCGGTGCACTACCGCCAG | |
| | TAAGTCTGCCTTAAAAAAAAAGCTGCCATCAGTCGGCGTGGAC | |
| | TGTAGAACACTGCCAATGCCGGTCCCAAGCCCGGATAAAAGT | |
| | GGAGGGTACAGTCCACGCTTTTTTTTACCGTGCAAGACGGAA | |
| | GCAGTTTGCTGACATTGCCTACAACTCCGCCAGTAAGTCTGCC | |
| | TTGCTTGTTGAGGGGTATGTGgctaagcAGCTTGGCGTAACTAGA | |
| | TCT | |
| Phenylketonuria; | ggccaaagcatgcatGAGCAAGGCAGACTTACTGGGTTTCAGAGCTA | 487 |
| not | TGCTGGAAACAGCATAGCAAGTTGAAATAAGGCTAGTCCGTTA | |
| provided | TCAACTTGAAAAAGTGGCACCGAGTCGGTGCTACAACTACCG | |
| | CCAGTAAGTCTGCCTTAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTGTGCAAGACGGAA | |
| | GCAGTTTGCTGACATTGCCTACAACTCCGCCAGTAAGTCTGCC | |
| | TTGCTTGTTGAGGGGATGTGCgctaagcAGCTTGGCGTAACTAG | |
| | ATCT | |
| Phenylketonuria; | ggccaaagcatgcatGCCATCCACCCAGGGAGAGAGTTTCAGAGCT | 488 |
| not | ATGCTGGAAACAGCATAGCAAGTTGAAATAAGGCTAGTCCGTT | |
| provided | ATCAACTTGAAAAAGTGGCACCGAGTCGGTGCGCCACAGTAA | |
| | GTCCCTTCTCTCCCTGGGTGGAAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTCCTGATGA | |
| | ATACATTGAAAAGCTCGCCGTAAGTCCCTTCTCTCCCTGGGTG | |
| | GATGGTGGAGTGCTAGCAAGAgctaagcAGCTTGGCGTAACTAG | |
| | ATCT | |
| Phenylketonuria; | ggccaaagcatgcatGCCATCCACCCAGGGAGAGAGTTTCAGAGCT | 489 |
| not | ATGCTGGAAACAGCATAGCAAGTTGAAATAAGGCTAGTCCGTT | |
| provided | ATCAACTTGAAAAAGTGGCACCGAGTCGGTGCAAAGCTCGCC | |
| | ACAGTAAGTCCCTTCTCTCCCTGGGTGGAAAAAAAAAAGCTGC | |
| | CATCAGTCGGCGTGGACTGTAGAACACTGCCAATGCCGGTCC | |
| | CAAGCCCGGATAAAAGTGGAGGGTACAGTCCACGCTTTTTTTA | |
| | ATACATTGAAAAGCTCGCCGTAAGTCCCTTCTCTCCCTGGGTG | |
| | GATGGTGGAGTGCTAGTAGCAgctaagcAGCTTGGCGTAACTAG | |
| | ATCT | |
| Phenylketonuria; | ggccaaagcatgcatGCCATCCACCCAGGGAGAGAGTTTCAGAGCT | 490 |
| not | ATGCTGGAAACAGCATAGCAAGTTGAAATAAGGCTAGTCCGTT | |
| provided | ATCAACTTGAAAAAGTGGCACCGAGTCGGTGCATTGAAAAGCT | |
| | CGCCACAGTAAGTCCCTTCTCTCCCTGGGTGGAAAAAAAAAAG | |
| | CTGCCATCAGTCGGCGTGGACTGTAGAACACTGCCAATGCCG | |
| | GTCCCAAGCCCGGATAAAAGTGGAGGGTACAGTCCACGCTTT | |
| | TTTTATTGAAAAGCTCGCCGTAAGTCCCTTCTCTCCCTGGGTG | |
| | GATGGTGGAGTGCTATGGTTGgctaagcAGCTTGGCGTAACTAG | |
| | ATCT | |
| Phenylketonuria; | ggccaaagcatgcatGCATCCACCCAGGGAGAGAAGTTTCAGAGCT | 491 |
| not | ATGCTGGAAACAGCATAGCAAGTTGAAATAAGGCTAGTCCGTT | |
| provided | ATCAACTTGAAAAAGTGGCACCGAGTCGGTGCGCCACAGTAA | |
| | GTCCCTTCTCTCCCTGGGTGGAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCACCTGATG | |
| | AATACATTGAAAAGCTCGCCGTAAGTCCCTTCTCTCCCTGGGT | |
| | GGATGGTGGAGTGCTCAGTAAgctaagcAGCTTGGCGTAACTAG | |
| | ATCT | |
| Phenylketonuria; | ggccaaagcatgcatGCATCCACCCAGGGAGAGAAGTTTCAGAGCT | 492 |
| not | ATGCTGGAAACAGCATAGCAAGTTGAAATAAGGCTAGTCCGTT | |
| provided | ATCAACTTGAAAAAGTGGCACCGAGTCGGTGCGCTCGCCACA | |
| | GTAAGTCCCTTCTCTCCCTGGGTGGAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTTGATG | |
| | AATACATTGAAAAGCTCGCCGTAAGTCCCTTCTCTCCCTGGGT | |
| | GGATGGTGGAGTGCTGGAGGCgctaagcAGCTTGGCGTAACTAG | |
| | ATCT | |
| Phenylketonuria; | ggccaaagcatgcatGCATCCACCCAGGGAGAGAAGTTTCAGAGCT | 493 |
| not | ATGCTGGAAACAGCATAGCAAGTTGAAATAAGGCTAGTCCGTT | |
| provided | ATCAACTTGAAAAAGTGGCACCGAGTCGGTGCAAAGCTCGCC | |
| | ACAGTAAGTCCCTTCTCTCCCTGGGTGGAAAAAAAAAGCTGCC | |
| | ATCAGTCGGCGTGGACTGTAGAACACTGCCAATGCCGGTCCC | |
| | AAGCCCGGATAAAAGTGGAGGGTACAGTCCACGCTTTTTTTTG | |
| | AATACATTGAAAAGCTCGCCGTAAGTCCCTTCTCTCCCTGGGT | |
| | GGATGGTGGAGTGCTTTTTAAgctaagcAGCTTGGCGTAACTAGA | |
| | TCT | |
| Phenylketonuria; | ggccaaagcatgcatGCATCCACCCAGGGAGAGAAGTTTCAGAGCT | 494 |
| not | ATGCTGGAAACAGCATAGCAAGTTGAAATAAGGCTAGTCCGTT | |
| provided | ATCAACTTGAAAAAGTGGCACCGAGTCGGTGCATTGAAAAGCT | |
| | CGCCACAGTAAGTCCCTTCTCTCCCTGGGTGGAAAAAAAAAGC | |
| | TGCCATCAGTCGGCGTGGACTGTAGAACACTGCCAATGCCGG | |
| | TCCCAAGCCCGGATAAAAGTGGAGGGTACAGTCCACGCTTTTT | |
| | TTACATTGAAAAGCTCGCCGTAAGTCCCTTCTCTCCCTGGGTG | |
| | GATGGTGGAGTGCTAAACACgctaagcAGCTTGGCGTAACTAGA | |
| | TCT | |
| Phenylketonuria; | ggccaaagcatgcatGCAGGGAGAGAAGGGACTTAGTTTCAGAGCT | 495 |
| not | ATGCTGGAAACAGCATAGCAAGTTGAAATAAGGCTAGTCCGTT | |
| provided | ATCAACTTGAAAAAGTGGCACCGAGTCGGTGCGCCACAGTAA | |
| | GTCCCTTCTCTCCAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTCTTGCCTCTCTGGGTGC | |
| | ACCTGATGAATACATTGAAAAGCTCGCCGTAAGTCCCTTCTCT | |
| | CCCTGGGTGGATGGTTCTGGAgctaagcAGCTTGGCGTAACTAG | |
| | ATCT | |
| Phenylketonuria; | ggccaaagcatgcatGCAGGGAGAGAAGGGACTTAGTTTCAGAGCT | 496 |
| not | ATGCTGGAAACAGCATAGCAAGTTGAAATAAGGCTAGTCCGTT | |
| provided | ATCAACTTGAAAAAGTGGCACCGAGTCGGTGCGCTCGCCACA | |
| | GTAAGTCCCTTCTCTCCAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTCCTCTCTGGGTGC | |
| | ACCTGATGAATACATTGAAAAGCTCGCCGTAAGTCCCTTCTCT | |
| | CCCTGGGTGGATGGTACAGTAgctaagcAGCTTGGCGTAACTAG | |
| | ATCT | |
| Phenylketonuria; | ggccaaagcatgcatGCAGGGAGAGAAGGGACTTAGTTTCAGAGCT | 497 |
| not | ATGCTGGAAACAGCATAGCAAGTTGAAATAAGGCTAGTCCGTT | |
| provided | ATCAACTTGAAAAAGTGGCACCGAGTCGGTGCAAAGCTCGCC | |
| | ACAGTAAGTCCCTTCTCTCCAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTCTCTGGGTGC | |
| | ACCTGATGAATACATTGAAAAGCTCGCCGTAAGTCCCTTCTCT | |
| | CCCTGGGTGGATGGTGGGCCCgctaagcAGCTTGGCGTAACTAG | |
| | ATCT | |
| Phenylketonuria; | ggccaaagcatgcatGCAGGGAGAGAAGGGACTTAGTTTCAGAGCT | 498 |
| not | ATGCTGGAAACAGCATAGCAAGTTGAAATAAGGCTAGTCCGTT | |
| provided | ATCAACTTGAAAAAGTGGCACCGAGTCGGTGCATTGAAAAGCT | |
| | CGCCACAGTAAGTCCCTTCTCTCCAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTGGTGCA | |
| | CCTGATGAATACATTGAAAAGCTCGCCGTAAGTCCCTTCTCTC | |
| | CCTGGGTGGATGGTTATTTGgctaagcAGCTTGGCGTAACTAGAT | |
| | CT | |
| Citrin | ggccaaagcatgcatGCTAACAGGTATTGAGCATGGTTTCAGAGCTA | 499 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCGAGGTTAGTGC | |
| type II; | CACATGCTCAATACCTGTAAAAAAAAAGCTGCCATCAGTCGGC | |
| Neonatal | GTGGACTGTAGAACACTGCCAATGCCGGTCCCAAGCCCGGAT | |
| intrahepatic | AAAAGTGGAGGGTACAGTCCACGCTTTTTTTGCTATGAAGGCT | |
| cholestasis | TCTTTGGACTGTATAGAGATTAGTGCCACATGCTCAATACCTG | |
| caused by | TTAGGTGAAATAACTCTATTgctaagcAGCTTGGCGTAACTAGAT | |
| citrin | CT | |
| deficiency | | |
| Citrin | ggccaaagcatgcatGCTAACAGGTATTGAGCATGGTTTCAGAGCTA | 500 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCTATAGAGGTTAG | |
| type II; | TGCCACATGCTCAATACCTGTAAAAAAAAAGCTGCCATCAGTC | |
| Neonatal | GGCGTGGACTGTAGAACACTGCCAATGCCGGTCCCAAGCCCG | |
| intrahepatic | GATAAAAGTGGAGGGTACAGTCCACGCTTTTTTTTGAAGGCTT | |
| cholestasis | CTTTGGACTGTATAGAGATTAGTGCCACATGCTCAATACCTGT | |
| caused by | TAGGTGAAATAACAGGCGCgctaagcAGCTTGGCGTAACTAGAT | |
| citrin | CT | |
| deficiency | | |
| Citrin | ggccaaagcatgcatGCTAACAGGTATTGAGCATGGTTTCAGAGCTA | 501 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCCTGTATAGAGG | |
| type II; | TTAGTGCCACATGCTCAATACCTGTAAAAAAAAAGCTGCCATC | |
| Neonatal | AGTCGGCGTGGACTGTAGAACACTGCCAATGCCGGTCCCAAG | |
| intrahepatic | CCCGGATAAAAGTGGAGGGTACAGTCCACGCTTTTTTTAGGCT | |
| cholestasis | TCTTTGGACTGTATAGAGATTAGTGCCACATGCTCAATACCTG | |
| caused by | TTAGGTGAAATAACTGTTTGgctaagcAGCTTGGCGTAACTAGAT | |
| citrin | CT | |
| deficiency | | |
| Citrin | ggccaaagcatgcatGCTAACAGGTATTGAGCATGGTTTCAGAGCTA | 502 |
| deficiency; | TGCTGGAAACAGCATAGCAAGTTGAAATAAGGCTAGTCCGTTA | |
| Citrullinemia | TCAACTTGAAAAAGTGGCACCGAGTCGGTGCTTGGACTGTATA | |
| type II; | GAGGTTAGTGCCACATGCTCAATACCTGTAAAAAAAAAGCTGC | |
| Neonatal | CATCAGTCGGCGTGGACTGTAGAACACTGCCAATGCCGGTCC | |
| intrahepatic | CAAGCCCGGATAAAAGTGGAGGGTACAGTCCACGCTTTTTTTT | |
| cholestasis | CTTTGGACTGTATAGAGATTAGTGCCACATGCTCAATACCTGT | |
| caused by | TAGGTGAAATAACGCGAGGgctaagcAGCTTGGCGTAACTAGAT | |
| citrin | CT | |
| deficiency | | |
| Argininosuccinate | ggccaaagcatgcatGGAGTCTCGGGTCAGTGCCAGTTTCAGAGCT | 503 |
| lyase | ATGCTGGAAACAGCATAGCAAGTTGAAATAAGGCTAGTCCGTT | |
| deficiency; | ATCAACTTGAAAAAGTGGCACCGAGTCGGTGCCAGCCACGCC | |
| not provided | GTGGCACTGACCCGAGAAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTCTGCCCTGACCC | |
| | TCCTGCCCCTGGCTTCCCACAGCCACGCCATGGCACTGACCC | |
| | GAGACTCTGAGCGGCTGGAGTTTgctaagcAGCTTGGCGTAACT | |
| | AGATCT | |
| Argininosuccinate | ggccaaagcatgcatGGAGTCTCGGGTCAGTGCCAGTTTCAGAGCT | 504 |
| lyase | ATGCTGGAAACAGCATAGCAAGTTGAAATAAGGCTAGTCCGTT | |
| deficiency; | ATCAACTTGAAAAAGTGGCACCGAGTCGGTGCTCCCACAGCC | |
| not provided | ACGCCGTGGCACTGACCCGAGAAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTCTGACCC | |
| | TCCTGCCCCTGGCTTCCCACAGCCACGCCATGGCACTGACCC | |
| | GAGACTCTGAGCGGCTGAGCAGAgctaagcAGCTTGGCGTAACT | |
| | AGATCT | |
| Arginase | ggccaaagcatgcatGTTTTAATTGTTCAGCCATGGTTTCAGAGCTAT | 505 |
| deficiency; | GCTGGAAACAGCATAGCAAGTTGAAATAAGGCTAGTCCGTTAT | |
| not provided | CAACTTGAAAAAGTGGCACCGAGTCGGTGCCTCGTGGCTGAA | |
| | CAATTAAAAAAAAAAGCTGCCATCAGTCGGCGTGGACTGTAGA | |
| | ACACTGCCAATGCCGGTCCCAAGCCCGGATAAAAGTGGAGGG | |
| | TACAGTCCACGCTTTTTTTTCAAGCAGACCAGCCTTTCTCAATA | |
| | CTGTAGGGCCTTCTTCCACCCCTCCTCATGGCTGAACAATTAA | |
| | AAAATAAAGTACCGGACAgctaagcAGCTTGGCGTAACTAGATCT | |
| Arginase | ggccaaagcatgcatGTTTTAATTGTTCAGCCATGGTTTCAGAGCTAT | 506 |
| deficiency; | GCTGGAAACAGCATAGCAAGTTGAAATAAGGCTAGTCCGTTAT | |
| not provided | CAACTTGAAAAAGTGGCACCGAGTCGGTGCCCTCCTCGTGGC | |
| | TGAACAATTAAAAAAAAAAGCTGCCATCAGTCGGCGTGGACTG | |
| | TAGAACACTGCCAATGCCGGTCCCAAGCCCGGATAAAAGTGG | |
| | AGGGTACAGTCCACGCTTTTTTTGCAGACCAGCCTTTCTCAAT | |
| | ACTGTAGGGCCTTCTTCCACCCCTCCTCATGGCTGAACAATTA | |
| | AAAAATAAAGTACGTCCTGgctaagcAGCTTGGCGTAACTAGATC | |
| | T | |
| Arginase | ggccaaagcatgcatGTTTTAATTGTTCAGCCATGGTTTCAGAGCTAT | 507 |
| deficiency; | GCTGGAAACAGCATAGCAAGTTGAAATAAGGCTAGTCCGTTAT | |
| not provided | CAACTTGAAAAAGTGGCACCGAGTCGGTGCCTTCCACCCCTC | |
| | CTCGTGGCTGAACAATTAAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTGCCTTTCTCAAT | |
| | ACTGTAGGGCCTTCTTCCACCCCTCCTCATGGCTGAACAATTA | |
| | AAAAATAAAGTACAAGGTGgctaagcAGCTTGGCGTAACTAGATC | |
| | T | |
| Arginase | ggccaaagcatgcatGCTTCTTCCACCCCTCCTCAGTTTCAGAGCTA | 508 |
| deficiency; | TGCTGGAAACAGCATAGCAAGTTGAAATAAGGCTAGTCCGTTA | |
| not provided | TCAACTTGAAAAAGTGGCACCGAGTCGGTGCCCACGAGGAGG | |
| | GGTGGAAGAAAAAAAAAGCTGCCATCAGTCGGCGTGGACTGT | |
| | AGAACACTGCCAATGCCGGTCCCAAGCCCGGATAAAAGTGGA | |
| | GGGTACAGTCCACGCTTTTTTTTGATGGTCATGATAATGTCTG | |
| | AAGTACTTTATTTTTTAATTGTTCAGCCATGAGGAGGGGTGGA | |
| | AGAAGGCCCTACAGTGATTGCgctaagcAGCTTGGCGTAACTAG | |
| | ATCT | |
| Arginase | ggccaaagcatgcatGCTTCTTCCACCCCTCCTCAGTTTCAGAGCTA | 509 |
| deficiency; | TGCTGGAAACAGCATAGCAAGTTGAAATAAGGCTAGTCCGTTA | |
| not provided | TCAACTTGAAAAAGTGGCACCGAGTCGGTGCTCAGCCACGAG | |
| | GAGGGGTGGAAGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTGGTCATGATAATGTCTG | |
| | AAGTACTTTATTTTTTAATTGTTCAGCCATGAGGAGGGGTGGA | |
| | AGAAGGCCCTACAGTGTGCTGgctaagcAGCTTGGCGTAACTAG | |
| | ATCT | |
| Arginase | ggccaaagcatgcatGCTTCTTCCACCCCTCCTCAGTTTCAGAGCTA | 510 |
| deficiency; | TGCTGGAAACAGCATAGCAAGTTGAAATAAGGCTAGTCCGTTA | |
| not provided | TCAACTTGAAAAAGTGGCACCGAGTCGGTGCTGTTCAGCCAC | |
| | GAGGAGGGGTGGAAGAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTCATGATAATGTCTG | |
| | AAGTACTTTATTTTTTAATTGTTCAGCCATGAGGAGGGGTGGA | |
| | AGAAGGCCCTACAGTCCGGAAgctaagcAGCTTGGCGTAACTAG | |
| | ATCT | |
| Arginase | ggccaaagcatgcatGCTTCTTCCACCCCTCCTCAGTTTCAGAGCTA | 511 |
| deficiency; | TGCTGGAAACAGCATAGCAAGTTGAAATAAGGCTAGTCCGTTA | |
| not provided | TCAACTTGAAAAAGTGGCACCGAGTCGGTGCTTAATTGTTCAG | |
| | CCACGAGGAGGGGTGGAAGAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTAATGTCTGAA | |
| | GTACTTTATTTTTTAATTGTTCAGCCATGAGGAGGGGTGGAAG | |
| | AAGGCCCTACAGTGGCACGgctaagcAGCTTGGCGTAACTAGAT | |
| | CT | |
| Ornithine | ggccaaagcatgcatGAGCATGGCAGATGCAGTATGTTTCAGAGCTA | 512 |
| carbamoyl- | TGCTGGAAACAGCATAGCAAGTTGAAATAAGGCTAGTCCGTTA | |
| transferase | TCAACTTGAAAAAGTGGCACCGAGTCGGTGCACTCGAGCCAA | |
| deficiency; | TACTGCATCTGCCATAAAAAAAAAGCTGCCATCAGTCGGCGTG | |
| not provided | GACTGTAGAACACTGCCAATGCCGGTCCCAAGCCCGGATAAA | |
| | AGTGGAGGGTACAGTCCACGCTTTTTTTTTAGCCAGGGTGTCC | |
| | AAATCTGATTGTTTATACACTTGAGCCAATACTGCATCTGCCAT | |
| | GCTAGACAATACACATTATgctaagcAGCTTGGCGTAACTAGATC | |
| | T | |
| Ornithine | ggccaaagcatgcatGAGCATGGCAGATGCAGTATGTTTCAGAGCTA | 513 |
| carbamoyl- | TGCTGGAAACAGCATAGCAAGTTGAAATAAGGCTAGTCCGTTA | |
| transferase | TCAACTTGAAAAAGTGGCACCGAGTCGGTGCATACACTCGAG | |
| deficiency; | CCAATACTGCATCTGCCATAAAAAAAAAGCTGCCATCAGTCGG | |
| not provided | CGTGGACTGTAGAACACTGCCAATGCCGGTCCCAAGCCCGGA | |
| | TAAAAGTGGAGGGTACAGTCCACGCTTTTTTTCCAGGGTGTCC | |
| | AAATCTGATTGTTTATACACTTGAGCCAATACTGCATCTGCCAT | |
| | GCTAGACAATACACGTCTTgctaagcAGCTTGGCGTAACTAGATC | |
| | T | |
| Ornithine | ggccaaagcatgcatGAGCATGGCAGATGCAGTATGTTTCAGAGCTA | 514 |
| carbamoyl- | TGCTGGAAACAGCATAGCAAGTTGAAATAAGGCTAGTCCGTTA | |
| transferase | TCAACTTGAAAAAGTGGCACCGAGTCGGTGCTTTATACACTCG | |
| deficiency; | AGCCAATACTGCATCTGCCATAAAAAAAAAGCTGCCATCAGTC | |
| not provided | GGCGTGGACTGTAGAACACTGCCAATGCCGGTCCCAAGCCCG | |
| | GATAAAAGTGGAGGGTACAGTCCACGCTTTTTTTGGGTGTCCA | |
| | AATCTGATTGTTTATACACTTGAGCCAATACTGCATCTGCCATG | |
| | CTAGACAATACAACGGCAgctaagcAGCTTGGCGTAACTAGATCT | |
| Ornithine | ggccaaagcatgcatGAGCATGGCAGATGCAGTATGTTTCAGAGCTA | 515 |
| carbamoyl- | TGCTGGAAACAGCATAGCAAGTTGAAATAAGGCTAGTCCGTTA | |
| transferase | TCAACTTGAAAAAGTGGCACCGAGTCGGTGCGATTGTTTATAC | |
| deficiency; | ACTCGAGCCAATACTGCATCTGCCATAAAAAAAAAGCTGCCAT | |
| not provided | CAGTCGGCGTGGACTGTAGAACACTGCCAATGCCGGTCCCAA | |
| | GCCCGGATAAAAGTGGAGGGTACAGTCCACGCTTTTTTTTCCA | |
| | AATCTGATTGTTTATACACTTGAGCCAATACTGCATCTGCCATG | |
| | CTAGACAATACACGGAGTgctaagcAGCTTGGCGTAACTAGATCT | |
| Arginase | ggccaaagcatgcatGCAGGAGAATCCTGGCACATGTTTCAGAGCTA | 516 |
| deficiency; | TGCTGGAAACAGCATAGCAAGTTGAAATAAGGCTAGTCCGTTA | |
| not provided | TCAACTTGAAAAAGTGGCACCGAGTCGGTGCTTAGATTCCCGA | |
| | TGTGCCAGGATTCTCAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTTCATACATAACCAAG | |
| | TGAAAACATTGTAATTTTACATTCCCGATGTGCCAGGATTCTCC | |
| | TGGGTGACTCCCTCCATGgctaagcAGCTTGGCGTAACTAGATCT | |
| Arginase | ggccaaagcatgcatGCAGGAGAATCCTGGCACATGTTTCAGAGCTA | 517 |
| deficiency; | TGCTGGAAACAGCATAGCAAGTTGAAATAAGGCTAGTCCGTTA | |
| not provided | TCAACTTGAAAAAGTGGCACCGAGTCGGTGCAATTTTAGATTC | |
| | CCGATGTGCCAGGATTCTCAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTACATAACCAAG | |
| | TGAAAACATTGTAATTTTACATTCCCGATGTGCCAGGATTCTCC | |
| | TGGGTGACTCCCAACTTAgctaagcAGCTTGGCGTAACTAGATCT | |
| Arginase | ggccaaagcatgcatGCAGGAGAATCCTGGCACATGTTTCAGAGCTA | 518 |
| deficiency; | TGCTGGAAACAGCATAGCAAGTTGAAATAAGGCTAGTCCGTTA | |
| not provided | TCAACTTGAAAAAGTGGCACCGAGTCGGTGCTGTAATTTTAGA | |
| | TTCCCGATGTGCCAGGATTCTCAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTTAACCAAG | |
| | TGAAAACATTGTAATTTTACATTCCCGATGTGCCAGGATTCTCC | |
| | TGGGTGACTCCCCAAGAGgctaagcAGCTTGGCGTAACTAGATC | |
| | T | |
| Arginase | ggccaaagcatgcatGCAGGAGAATCCTGGCACATGTTTCAGAGCTA | 519 |
| deficiency; | TGCTGGAAACAGCATAGCAAGTTGAAATAAGGCTAGTCCGTTA | |
| not provided | TCAACTTGAAAAAGTGGCACCGAGTCGGTGCAACATTGTAATT | |
| | TTAGATTCCCGATGTGCCAGGATTCTCAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTAAG | |
| | TGAAAACATTGTAATTTTACATTCCCGATGTGCCAGGATTCTCC | |
| | TGGGTGACTCCCCTGGCCgctaagcAGCTTGGCGTAACTAGATC | |
| | T | |
| Arginase | ggccaaagcatgcatGAGGAGAATCCTGGCACATCGTTTCAGAGCTA | 520 |
| deficiency; | TGCTGGAAACAGCATAGCAAGTTGAAATAAGGCTAGTCCGTTA | |
| not provided | TCAACTTGAAAAAGTGGCACCGAGTCGGTGCTTAGATTCCCGA | |
| | TGTGCCAGGATTCTAAAAAAAAAGCTGCCATCAGTCGGCGTG | |
| | GACTGTAGAACACTGCCAATGCCGGTCCCAAGCCCGGATAAA | |
| | AGTGGAGGGTACAGTCCACGCTTTTTTTAATCATACATAACCA | |
| | AGTGAAAACATTGTAATTTTACATTCCCGATGTGCCAGGATTCT | |
| | CCTGGGTGACTCCCGAAAAgctaagcAGCTTGGCGTAACTAGAT | |
| | CT | |
| Arginase | ggccaaagcatgcatGAGGAGAATCCTGGCACATCGTTTCAGAGCTA | 521 |
| deficiency; | TGCTGGAAACAGCATAGCAAGTTGAAATAAGGCTAGTCCGTTA | |
| not provided | TCAACTTGAAAAAGTGGCACCGAGTCGGTGCAATTTTAGATTC | |
| | CCGATGTGCCAGGATTCTAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTATACATAACCAA | |
| | GTGAAAACATTGTAATTTTACATTCCCGATGTGCCAGGATTCTC | |
| | CTGGGTGACTCCTAGCCAgctaagcAGCTTGGCGTAACTAGATCT | |
| Arginase | ggccaaagcatgcatGAGGAGAATCCTGGCACATCGTTTCAGAGCTA | 522 |
| deficiency; | TGCTGGAAACAGCATAGCAAGTTGAAATAAGGCTAGTCCGTTA | |
| not provided | TCAACTTGAAAAAGTGGCACCGAGTCGGTGCTGTAATTTTAGA | |
| | TTCCCGATGTGCCAGGATTCTAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTCATAACCAA | |
| | GTGAAAACATTGTAATTTTACATTCCCGATGTGCCAGGATTCTC | |
| | CTGGGTGACTCCAGAAGAgctaagcAGCTTGGCGTAACTAGATC | |
| | T | |
| Arginase | ggccaaagcatgcatGAGGAGAATCCTGGCACATCGTTTCAGAGCTA | 523 |
| deficiency; | TGCTGGAAACAGCATAGCAAGTTGAAATAAGGCTAGTCCGTTA | |
| not provided | TCAACTTGAAAAAGTGGCACCGAGTCGGTGCAACATTGTAATT | |
| | TTAGATTCCCGATGTGCCAGGATTCTAAAAAAAAAGCTGCCAT | |
| | CAGTCGGCGTGGACTGTAGAACACTGCCAATGCCGGTCCCAA | |
| | GCCCGGATAAAAGTGGAGGGTACAGTCCACGCTTTTTTTCCAA | |
| | GTGAAAACATTGTAATTTTACATTCCCGATGTGCCAGGATTCTC | |
| | CTGGGTGACTCCTACGGCgctaagcAGCTTGGCGTAACTAGATC | |
| | T | |
| Ornithine | ggccaaagcatgcatGCATCCATCCCAATTATCAGGTTTCAGAGCTA | 524 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCCATTGATAATT | |
| deficiency; | GGGATGGAAAAAAAAAGCTGCCATCAGTCGGCGTGGACTGTA | |
| not provided | GAACACTGCCAATGCCGGTCCCAAGCCCGGATAAAAGTGGAG | |
| | GGTACAGTCCACGCTTTTTTTTGAGGTAATCAGCCAGGATCTG | |
| | GATAGGATGGTACAAATCTGACAGCCCACTGATAATTGGGATG | |
| | GATGCTTCTTTAGCACCGGGgctaagcAGCTTGGCGTAACTAGAT | |
| | CT | |
| Ornithine | ggccaaagcatgcatGCATCCATCCCAATTATCAGGTTTCAGAGCTA | 525 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCAGCCCATTGA | |
| deficiency; | TAATTGGGATGGAAAAAAAAAGCTGCCATCAGTCGGCGTGGA | |
| not provided | CTGTAGAACACTGCCAATGCCGGTCCCAAGCCCGGATAAAAG | |
| | TGGAGGGTACAGTCCACGCTTTTTTTGTAATCAGCCAGGATCT | |
| | GGATAGGATGGTACAAATCTGACAGCCCACTGATAATTGGGAT | |
| | GGATGCTTCTTTAGCCCAAATgctaagcAGCTTGGCGTAACTAGA | |
| | TCT | |
| Ornithine | ggccaaagcatgcatGCATCCATCCCAATTATCAGGTTTCAGAGCTA | 526 |
| carbamoyl- | TGCTGGAAACAGCATAGCAAGTTGAAATAAGGCTAGTCCGTTA | |
| transferase | TCAACTTGAAAAAGTGGCACCGAGTCGGTGCTGACAGCCCAT | |
| deficiency; | TGATAATTGGGATGGAAAAAAAAAGCTGCCATCAGTCGGCGTG | |
| not provided | GACTGTAGAACACTGCCAATGCCGGTCCCAAGCCCGGATAAA | |
| | AGTGGAGGGTACAGTCCACGCTTTTTTTATCAGCCAGGATCTG | |
| | GATAGGATGGTACAAATCTGACAGCCCACTGATAATTGGGATG | |
| | GATGCTTCTTTAGCTATAACgctaagcAGCTTGGCGTAACTAGAT | |
| | CT | |
| Ornithine | ggccaaagcatgcatGCATCCATCCCAATTATCAGGTTTCAGAGCTA | 527 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAAATCTGACAG | |
| deficiency; | CCCATTGATAATTGGGATGGAAAAAAAAAGCTGCCATCAGTCG | |
| not provided | GCGTGGACTGTAGAACACTGCCAATGCCGGTCCCAAGCCCGG | |
| | ATAAAAGTGGAGGGTACAGTCCACGCTTTTTTTCCAGGATCTG | |
| | GATAGGATGGTACAAATCTGACAGCCCACTGATAATTGGGATG | |
| | GATGCTTCTTTAGCGACCTAgctaagcAGCTTGGCGTAACTAGAT | |
| | CT | |
| Ornithine | ggccaaagcatgcatGATCCATCCCAATTATCAGTGTTTCAGAGCTAT | 528 |
| carbamoyl- | GCTGGAAACAGCATAGCAAGTTGAAATAAGGCTAGTCCGTTAT | |
| transferase | CAACTTGAAAAAGTGGCACCGAGTCGGTGCCCATTGATAATTG | |
| deficiency; | GGATGAAAAAAAAAGCTGCCATCAGTCGGCGTGGACTGTAGA | |
| not provided | ACACTGCCAATGCCGGTCCCAAGCCCGGATAAAAGTGGAGGG | |
| | TACAGTCCACGCTTTTTTTCGTGAGGTAATCAGCCAGGATCTG | |
| | GATAGGATGGTACAAATCTGACAGCCCACTGATAATTGGGATG | |
| | GATGCTTCTTTAGCACCATgctaagcAGCTTGGCGTAACTAGATC | |
| | T | |
| Ornithine | ggccaaagcatgcatGATCCATCCCAATTATCAGTGTTTCAGAGCTAT | 529 |
| carbamoyl- | GCTGGAAACAGCATAGCAAGTTGAAATAAGGCTAGTCCGTTAT | |
| transferase | CAACTTGAAAAAGTGGCACCGAGTCGGTGCCAGCCCATTGAT | |
| deficiency; | AATTGGGATGAAAAAAAAAGCTGCCATCAGTCGGCGTGGACT | |
| not provided | GTAGAACACTGCCAATGCCGGTCCCAAGCCCGGATAAAAGTG | |
| | GAGGGTACAGTCCACGCTTTTTTTAGGTAATCAGCCAGGATCT | |
| | GGATAGGATGGTACAAATCTGACAGCCCACTGATAATTGGGAT | |
| | GGATGCTTCTTTAGCGCGCCgctaagcAGCTTGGCGTAACTAGAT | |
| | CT | |
| Ornithine | ggccaaagcatgcatGATCCATCCCAATTATCAGTGTTTCAGAGCTAT | 530 |
| carbamoyl- | GCTGGAAACAGCATAGCAAGTTGAAATAAGGCTAGTCCGTTAT | |
| transferase | CAACTTGAAAAAGTGGCACCGAGTCGGTGCTGACAGCCCATT | |
| deficiency; | GATAATTGGGATGAAAAAAAAAGCTGCCATCAGTCGGCGTGG | |
| not provided | ACTGTAGAACACTGCCAATGCCGGTCCCAAGCCCGGATAAAA | |
| | GTGGAGGGTACAGTCCACGCTTTTTTTTAATCAGCCAGGATCT | |
| | GGATAGGATGGTACAAATCTGACAGCCCACTGATAATTGGGAT | |
| | GGATGCTTCTTTAGGTAGGCgctaagcAGCTTGGCGTAACTAGAT | |
| | CT | |
| Ornithine | ggccaaagcatgcatGATCCATCCCAATTATCAGTGTTTCAGAGCTAT | 531 |
| carbamoyl- | GCTGGAAACAGCATAGCAAGTTGAAATAAGGCTAGTCCGTTAT | |
| transferase | CAACTTGAAAAAGTGGCACCGAGTCGGTGCAAATCTGACAGC | |
| deficiency; | CCATTGATAATTGGGATGAAAAAAAAAGCTGCCATCAGTCGGC | |
| not provided | GTGGACTGTAGAACACTGCCAATGCCGGTCCCAAGCCCGGAT | |
| | AAAAGTGGAGGGTACAGTCCACGCTTTTTTTAGCCAGGATCTG | |
| | GATAGGATGGTACAAATCTGACAGCCCACTGATAATTGGGATG | |
| | GATGCTTCTTTAGCATATCgctaagcAGCTTGGCGTAACTAGATC | |
| | T | |
| Abnormality | ggccaaagcatgcatGTCATTCATTGTGTTAAATAGTTTCAGAGCTAT | 532 |
| of ornithine | GCTGGAAACAGCATAGCAAGTTGAAATAAGGCTAGTCCGTTAT | |
| metabolism; | CAACTTGAAAAAGTGGCACCGAGTCGGTGCTTCGGTGCCATA | |
| Hyperammonemia; | TTTAACACAATGAAAAAAAAAAAGCTGCCATCAGTCGGCGTGG | |
| Ornithine | ACTGTAGAACACTGCCAATGCCGGTCCCAAGCCCGGATAAAA | |
| carbamoyl- | GTGGAGGGTACAGTCCACGCTTTTTTTTCTCATCTTATGTTCTG | |
| transferase | TCAATCCCTTTGATTTTTCAGTGCCATATTTAACACAATGAATG | |
| deficiency; | AATAGAGAACATGAGTAgctaagcAGCTTGGCGTAACTAGATCT | |
| Protein | | |
| avoidance; | | |
| not provided | | |
| Abnormality | ggccaaagcatgcatGTCATTCATTGTGTTAAATAGTTTCAGAGCTAT | 533 |
| of ornithine | GCTGGAAACAGCATAGCAAGTTGAAATAAGGCTAGTCCGTTAT | |
| metabolism; | CAACTTGAAAAAGTGGCACCGAGTCGGTGCATTTTTCGGTGCC | |
| Hyperammonemia; | ATATTTAACACAATGAAAAAAAAAAAGCTGCCATCAGTCGGCG | |
| Ornithine | TGGACTGTAGAACACTGCCAATGCCGGTCCCAAGCCCGGATA | |
| carbamoyl- | AAAGTGGAGGGTACAGTCCACGCTTTTTTTATCTTATGTTCTGT | |
| transferase | CAATCCCTTTGATTTTTCAGTGCCATATTTAACACAATGAATGA | |
| deficiency; | ATAGAGAACACGCCCCgctaagcAGCTTGGCGTAACTAGATCT | |
| Protein | | |
| avoidance; | | |
| not provided | | |
| Abnormality | ggccaaagcatgcatGTCATTCATTGTGTTAAATAGTTTCAGAGCTAT | 534 |
| of ornithine | GCTGGAAACAGCATAGCAAGTTGAAATAAGGCTAGTCCGTTAT | |
| metabolism; | CAACTTGAAAAAGTGGCACCGAGTCGGTGCTTGATTTTTCGGT | |
| Hyperammonemia; | GCCATATTTAACACAATGAAAAAAAAAAAGCTGCCATCAGTCG | |
| Ornithine | GCGTGGACTGTAGAACACTGCCAATGCCGGTCCCAAGCCCGG | |
| carbamoyl- | ATAAAAGTGGAGGGTACAGTCCACGCTTTTTTTTTATGTTCTGT | |
| transferase | CAATCCCTTTGATTTTTCAGTGCCATATTTAACACAATGAATGA | |
| deficiency; | ATAGAGAACATCTAGGgctaagcAGCTTGGCGTAACTAGATCT | |
| Protein | | |
| avoidance; | | |
| not provided | | |
| Abnormality | ggccaaagcatgcatGTCATTCATTGTGTTAAATAGTTTCAGAGCTAT | 535 |
| of ornithine | GCTGGAAACAGCATAGCAAGTTGAAATAAGGCTAGTCCGTTAT | |
| metabolism; | CAACTTGAAAAAGTGGCACCGAGTCGGTGCTCCCTTTGATTTT | |
| Hyperammonemia; | TCGGTGCCATATTTAACACAATGAAAAAAAAAAAGCTGCCATC | |
| Ornithine | AGTCGGCGTGGACTGTAGAACACTGCCAATGCCGGTCCCAAG | |
| carbamoyl- | CCCGGATAAAAGTGGAGGGTACAGTCCACGCTTTTTTTTTCTG | |
| transferase | TCAATCCCTTTGATTTTTCAGTGCCATATTTAACACAATGAATG | |
| deficiency; | AATAGAGAACATTTCAAgctaagcAGCTTGGCGTAACTAGATCT | |
| Protein | | |
| avoidance; | | |
| not provided | | |
| Phenylketonuria; | ggccaaagcatgcatGGGAGGAAGAAAAGAAAACAGTTTCAGAGCTA | 536 |
| not | TGCTGGAAACAGCATAGCAAGTTGAAATAAGGCTAGTCCGTTA | |
| provided | TCAACTTGAAAAAGTGGCACCGAGTCGGTGCAACACTGTGCC | |
| | CCATGTTTTCTTTTCTTCCAAAAAAAAAGCTGCCATCAGTCGGC | |
| | GTGGACTGTAGAACACTGCCAATGCCGGTCCCAAGCCCGGAT | |
| | AAAAGTGGAGGGTACAGTCCACGCTTTTTTTTTTATACAAGGA | |
| | CTTCAGAGTCTTGAACGCTGTGCCCCATGTTTTCTTTTCTTCCT | |
| | CCATGTATTCCAACACGGgctaagcAGCTTGGCGTAACTAGATCT | |
| Phenylketonuria; | ggccaaagcatgcatGGGAGGAAGAAAAGAAAACAGTTTCAGAGCTA | 537 |
| not | TGCTGGAAACAGCATAGCAAGTTGAAATAAGGCTAGTCCGTTA | |
| provided | TCAACTTGAAAAAGTGGCACCGAGTCGGTGCCTTGAACACTGT | |
| | GCCCCATGTTTTCTTTTCTTCCAAAAAAAAAGCTGCCATCAGTC | |
| | GGCGTGGACTGTAGAACACTGCCAATGCCGGTCCCAAGCCCG | |
| | GATAAAAGTGGAGGGTACAGTCCACGCTTTTTTTTACAAGGAC | |
| | TTCAGAGTCTTGAACGCTGTGCCCCATGTTTTCTTTTCTTCCTC | |
| | CATGTATTCCACATGAGgctaagcAGCTTGGCGTAACTAGATCT | |
| Phenylketonuria; | ggccaaagcatgcatGGGAGGAAGAAAAGAAAACAGTTTCAGAGCTA | 538 |
| not | TGCTGGAAACAGCATAGCAAGTTGAAATAAGGCTAGTCCGTTA | |
| provided | TCAACTTGAAAAAGTGGCACCGAGTCGGTGCAGTCTTGAACAC | |
| | TGTGCCCCATGTTTTCTTTTCTTCCAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTAAGGAC | |
| | TTCAGAGTCTTGAACGCTGTGCCCCATGTTTTCTTTTCTTCCTC | |
| | CATGTATTCCACACAATgctaagcAGCTTGGCGTAACTAGATCT | |
| Phenylketonuria; | ggccaaagcatgcatGGAGGAAGAAAAGAAAACATGTTTCAGAGCTA | 539 |
| not | TGCTGGAAACAGCATAGCAAGTTGAAATAAGGCTAGTCCGTTA | |
| provided | TCAACTTGAAAAAGTGGCACCGAGTCGGTGCAACACTGTGCC | |
| | CCATGTTTTCTTTTCTTCAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTGTTTTATACAAGG | |
| | ACTTCAGAGTCTTGAACGCTGTGCCCCATGTTTTCTTTTCTTCC | |
| | TCCATGTATTCCTGCACTgctaagcAGCTTGGCGTAACTAGATCT | |
| Phenylketonuria; | ggccaaagcatgcatGGAGGAAGAAAAGAAAACATGTTTCAGAGCTA | 540 |
| not | TGCTGGAAACAGCATAGCAAGTTGAAATAAGGCTAGTCCGTTA | |
| provided | TCAACTTGAAAAAGTGGCACCGAGTCGGTGCCTTGAACACTGT | |
| | GCCCCATGTTTTCTTTTCTTCAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTATACAAGGA | |
| | CTTCAGAGTCTTGAACGCTGTGCCCCATGTTTTCTTTTCTTCCT | |
| | CCATGTATTCCTGGCATgctaagcAGCTTGGCGTAACTAGATCT | |
| Phenylketonuria; | ggccaaagcatgcatGGAGGAAGAAAAGAAAACATGTTTCAGAGCTA | 541 |
| not | TGCTGGAAACAGCATAGCAAGTTGAAATAAGGCTAGTCCGTTA | |
| provided | TCAACTTGAAAAAGTGGCACCGAGTCGGTGCAGTCTTGAACAC | |
| | TGTGCCCCATGTTTTCTTTTCTTCAAAAAAAAAGCTGCCATCAG | |
| | TCGGCGTGGACTGTAGAACACTGCCAATGCCGGTCCCAAGCC | |
| | CGGATAAAAGTGGAGGGTACAGTCCACGCTTTTTTTACAAGGA | |
| | CTTCAGAGTCTTGAACGCTGTGCCCCATGTTTTCTTTTCTTCCT | |
| | CCATGTATTCCGTTCCTgctaagcAGCTTGGCGTAACTAGATCT | |
| Phenylketonuria; | ggccaaagcatgcatGGAGGAAGAAAAGAAAACATGTTTCAGAGCTA | 542 |
| not | TGCTGGAAACAGCATAGCAAGTTGAAATAAGGCTAGTCCGTTA | |
| provided | TCAACTTGAAAAAGTGGCACCGAGTCGGTGCTTCAGAGTCTTG | |
| | AACACTGTGCCCCATGTTTTCTTTTCTTCAAAAAAAAAGCTGCC | |
| | ATCAGTCGGCGTGGACTGTAGAACACTGCCAATGCCGGTCCC | |
| | AAGCCCGGATAAAAGTGGAGGGTACAGTCCACGCTTTTTTTGA | |
| | CTTCAGAGTCTTGAACGCTGTGCCCCATGTTTTCTTTTCTTCCT | |
| | CCATGTATTCCATAGCTgctaagcAGCTTGGCGTAACTAGATCT | |
| Phenylketonuria; | ggccaaagcatgcatGAGGAAGAAAAGAAAACATGGTTTCAGAGCTA | 543 |
| not | TGCTGGAAACAGCATAGCAAGTTGAAATAAGGCTAGTCCGTTA | |
| provided | TCAACTTGAAAAAGTGGCACCGAGTCGGTGCAACACTGTGCC | |
| | CCATGTTTTCTTTTCTTAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTGGGTTTTATACAAG | |
| | GACTTCAGAGTCTTGAACGCTGTGCCCCATGTTTTCTTTTCTTC | |
| | CTCCATGTATTCGTATGTgctaagcAGCTTGGCGTAACTAGATCT | |
| Phenylketonuria; | ggccaaagcatgcatGAGGAAGAAAAGAAAACATGGTTTCAGAGCTA | 544 |
| not | TGCTGGAAACAGCATAGCAAGTTGAAATAAGGCTAGTCCGTTA | |
| provided | TCAACTTGAAAAAGTGGCACCGAGTCGGTGCAGTCTTGAACAC | |
| | TGTGCCCCATGTTTTCTTTTCTTAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTATACAAGG | |
| | ACTTCAGAGTCTTGAACGCTGTGCCCCATGTTTTCTTTTCTTCC | |
| | TCCATGTATTCCCTAAGgctaagcAGCTTGGCGTAACTAGATCT | |
| Phenylketonuria; | ggccaaagcatgcatGAGGAAGAAAAGAAAACATGGTTTCAGAGCTA | 545 |
| not | TGCTGGAAACAGCATAGCAAGTTGAAATAAGGCTAGTCCGTTA | |
| provided | TCAACTTGAAAAAGTGGCACCGAGTCGGTGCTTCAGAGTCTTG | |
| | AACACTGTGCCCCATGTTTTCTTTTCTTAAAAAAAAAGCTGCCA | |
| | TCAGTCGGCGTGGACTGTAGAACACTGCCAATGCCGGTCCCA | |
| | AGCCCGGATAAAAGTGGAGGGTACAGTCCACGCTTTTTTTAGG | |
| | ACTTCAGAGTCTTGAACGCTGTGCCCCATGTTTTCTTTTCTTCC | |
| | TCCATGTATTCTTTCCAgctaagcAGCTTGGCGTAACTAGATCT | |
| Ornithine | ggccaaagcatgcatGAGGCAGCTACTCCAAAGTTGTTTCAGAGCTA | 546 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCTACCTTTGGA | |
| deficiency; | GTAGCTGAAAAAAAAAGCTGCCATCAGTCGGCGTGGACTGTA | |
| not provided | GAACACTGCCAATGCCGGTCCCAAGCCCGGATAAAAGTGGAG | |
| | GGTACAGTCCACGCTTTTTTTATCTGAGGATGATTTAAGAGAG | |
| | GGGTTAGTTTCAAGGCAAAAAGTTTCCCTAACTTTGGAGTAGC | |
| | TGCCTGAAGGTGCATGGTAATgctaagcAGCTTGGCGTAACTAGA | |
| | TCT | |
| Ornithine | ggccaaagcatgcatGAGGCAGCTACTCCAAAGTTGTTTCAGAGCTA | 547 |
| carbamoyl- | TGCTGGAAACAGCATAGCAAGTTGAAATAAGGCTAGTCCGTTA | |
| transferase | TCAACTTGAAAAAGTGGCACCGAGTCGGTGCTTCCCTACCTTT | |
| deficiency; | GGAGTAGCTGAAAAAAAAAGCTGCCATCAGTCGGCGTGGACT | |
| not provided | GTAGAACACTGCCAATGCCGGTCCCAAGCCCGGATAAAAGTG | |
| | GAGGGTACAGTCCACGCTTTTTTTGAGGATGATTTAAGAGAGG | |
| | GGTTAGTTTCAAGGCAAAAAGTTTCCCTAACTTTGGAGTAGCT | |
| | GCCTGAAGGTGCATATCACTgctaagcAGCTTGGCGTAACTAGAT | |
| | CT | |
| Ornithine | ggccaaagcatgcatGAGGCAGCTACTCCAAAGTTGTTTCAGAGCTA | 548 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAGTTTCCCTACC | |
| deficiency; | TTTGGAGTAGCTGAAAAAAAAAGCTGCCATCAGTCGGCGTGG | |
| not provided | ACTGTAGAACACTGCCAATGCCGGTCCCAAGCCCGGATAAAA | |
| | GTGGAGGGTACAGTCCACGCTTTTTTTGATGATTTAAGAGAGG | |
| | GGTTAGTTTCAAGGCAAAAAGTTTCCCTAACTTTGGAGTAGCT | |
| | GCCTGAAGGTGCATTCTCGCgctaagcAGCTTGGCGTAACTAGA | |
| | TCT | |
| Ornithine | ggccaaagcatgcatGAGGCAGCTACTCCAAAGTTGTTTCAGAGCTA | 549 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCAAAAAGTTTCC | |
| deficiency; | CTACCTTTGGAGTAGCTGAAAAAAAAAGCTGCCATCAGTCGGC | |
| not provided | GTGGACTGTAGAACACTGCCAATGCCGGTCCCAAGCCCGGAT | |
| | AAAAGTGGAGGGTACAGTCCACGCTTTTTTTTTTAAGAGAGGG | |
| | GTTAGTTTCAAGGCAAAAAGTTTCCCTAACTTTGGAGTAGCTG | |
| | CCTGAAGGTGCATTGACCGgctaagcAGCTTGGCGTAACTAGAT | |
| | CT | |
| Ornithine | ggccaaagcatgcatGGGCAGCTACTCCAAAGTTAGTTTCAGAGCTA | 550 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCTACCTTTGGA | |
| deficiency; | GTAGCTAAAAAAAAAGCTGCCATCAGTCGGCGTGGACTGTAG | |
| not provided | AACACTGCCAATGCCGGTCCCAAGCCCGGATAAAAGTGGAGG | |
| | GTACAGTCCACGCTTTTTTTGCATCTGAGGATGATTTAAGAGA | |
| | GGGGTTAGTTTCAAGGCAAAAAGTTTCCCTAACTTTGGAGTAG | |
| | CTGCCTGAAGGTGCATATGTTgctaagcAGCTTGGCGTAACTAGA | |
| | TCT | |
| Ornithine | ggccaaagcatgcatGGGCAGCTACTCCAAAGTTAGTTTCAGAGCTA | 551 |
| carbamoyl- | TGCTGGAAACAGCATAGCAAGTTGAAATAAGGCTAGTCCGTTA | |
| transferase | TCAACTTGAAAAAGTGGCACCGAGTCGGTGCTTCCCTACCTTT | |
| deficiency; | GGAGTAGCTAAAAAAAAAGCTGCCATCAGTCGGCGTGGACTG | |
| not provided | TAGAACACTGCCAATGCCGGTCCCAAGCCCGGATAAAAGTGG | |
| | AGGGTACAGTCCACGCTTTTTTTCTGAGGATGATTTAAGAGAG | |
| | GGGTTAGTTTCAAGGCAAAAAGTTTCCCTAACTTTGGAGTAGC | |
| | TGCCTGAAGGTGCAACAACCgctaagcAGCTTGGCGTAACTAGA | |
| | TCT | |
| Ornithine | ggccaaagcatgcatGGGCAGCTACTCCAAAGTTAGTTTCAGAGCTA | 552 |
| carbamoyl- | TGCTGGAAACAGCATAGCAAGTTGAAATAAGGCTAGTCCGTTA | |
| transferase | TCAACTTGAAAAAGTGGCACCGAGTCGGTGCAGTTTCCCTACC | |
| deficiency; | TTTGGAGTAGCTAAAAAAAAAGCTGCCATCAGTCGGCGTGGAC | |
| not provided | TGTAGAACACTGCCAATGCCGGTCCCAAGCCCGGATAAAAGT | |
| | GGAGGGTACAGTCCACGCTTTTTTTAGGATGATTTAAGAGAGG | |
| | GGTTAGTTTCAAGGCAAAAAGTTTCCCTAACTTTGGAGTAGCT | |
| | GCCTGAAGGTGCAGTCGCAgctaagcAGCTTGGCGTAACTAGAT | |
| | CT | |
| Ornithine | ggccaaagcatgcatGGGCAGCTACTCCAAAGTTAGTTTCAGAGCTA | 553 |
| carbamoyl- | TGCTGGAAACAGCATAGCAAGTTGAAATAAGGCTAGTCCGTTA | |
| transferase | TCAACTTGAAAAAGTGGCACCGAGTCGGTGCCAAAAAGTTTCC | |
| deficiency; | CTACCTTTGGAGTAGCTAAAAAAAAAGCTGCCATCAGTCGGCG | |
| not provided | TGGACTGTAGAACACTGCCAATGCCGGTCCCAAGCCCGGATA | |
| | AAAGTGGAGGGTACAGTCCACGCTTTTTTTGATTTAAGAGAGG | |
| | GGTTAGTTTCAAGGCAAAAAGTTTCCCTAACTTTGGAGTAGCT | |
| | GCCTGAAGGTGCACAGTCGgctaagcAGCTTGGCGTAACTAGAT | |
| | CT | |
| Phenylketonuria; | ggccaaagcatgcatGAGCTGGAGGACAGTACTCAGTTTCAGAGCTA | 554 |
| not | TGCTGGAAACAGCATAGCAAGTTGAAATAAGGCTAGTCCGTTA | |
| provided | TCAACTTGAAAAAGTGGCACCGAGTCGGTGCATACCCCCGAA | |
| | CCGTGAGTACTGTCCTCCAAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTATCAGACATGG | |
| | ATCCAAGCCCATGTATATCCCCGAACCGTGAGTACTGTCCTCC | |
| | AGCTACCAGTTGCCGGCCGAgctaagcAGCTTGGCGTAACTAGA | |
| | TCT | |
| Phenylketonuria; | ggccaaagcatgcatGAGCTGGAGGACAGTACTCAGTTTCAGAGCTA | 555 |
| not | TGCTGGAAACAGCATAGCAAGTTGAAATAAGGCTAGTCCGTTA | |
| provided | TCAACTTGAAAAAGTGGCACCGAGTCGGTGCATGTATACCCCC | |
| | GAACCGTGAGTACTGTCCTCCAAAAAAAAAAGCTGCCATCAGT | |
| | CGGCGTGGACTGTAGAACACTGCCAATGCCGGTCCCAAGCCC | |
| | GGATAAAAGTGGAGGGTACAGTCCACGCTTTTTTTGACATGGA | |
| | TCCAAGCCCATGTATATCCCCGAACCGTGAGTACTGTCCTCCA | |
| | GCTACCAGTTGCCCGTGCAgctaagcAGCTTGGCGTAACTAGAT | |
| | CT | |
| Phenylketonuria; | ggccaaagcatgcatGAGCTGGAGGACAGTACTCAGTTTCAGAGCTA | 556 |
| not | TGCTGGAAACAGCATAGCAAGTTGAAATAAGGCTAGTCCGTTA | |
| provided | TCAACTTGAAAAAGTGGCACCGAGTCGGTGCCCAAGCCCATG | |
| | TATACCCCCGAACCGTGAGTACTGTCCTCCAAAAAAAAAAGCT | |
| | GCCATCAGTCGGCGTGGACTGTAGAACACTGCCAATGCCGGT | |
| | CCCAAGCCCGGATAAAAGTGGAGGGTACAGTCCACGCTTTTTT | |
| | TTCCAAGCCCATGTATATCCCCGAACCGTGAGTACTGTCCTCC | |
| | AGCTACCAGTTGCCACTTTGgctaagcAGCTTGGCGTAACTAGAT | |
| | CT | |
| Phenylketonuria; | ggccaaagcatgcatGAGGACAGTACTCACGGTTCGTTTCAGAGCTA | 557 |
| not | TGCTGGAAACAGCATAGCAAGTTGAAATAAGGCTAGTCCGTTA | |
| provided | TCAACTTGAAAAAGTGGCACCGAGTCGGTGCATACCCCCGAA | |
| | CCGTGAGTACTGTAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTTGCACACAGTACATCA | |
| | GACATGGATCCAAGCCCATGTATATCCCCGAACCGTGAGTACT | |
| | GTCCTCCAGCTACCAGCCAAAgctaagcAGCTTGGCGTAACTAG | |
| | ATCT | |
| Phenylketonuria; | ggccaaagcatgcatGAGGACAGTACTCACGGTTCGTTTCAGAGCTA | 558 |
| not | TGCTGGAAACAGCATAGCAAGTTGAAATAAGGCTAGTCCGTTA | |
| provided | TCAACTTGAAAAAGTGGCACCGAGTCGGTGCATGTATACCCCC | |
| | GAACCGTGAGTACTGTAAAAAAAAAGCTGCCATCAGTCGGCGT | |
| | GGACTGTAGAACACTGCCAATGCCGGTCCCAAGCCCGGATAA | |
| | AAGTGGAGGGTACAGTCCACGCTTTTTTTCACAGTACATCAGA | |
| | CATGGATCCAAGCCCATGTATATCCCCGAACCGTGAGTACTGT | |
| | CCTCCAGCTACCACAACCTgctaagcAGCTTGGCGTAACTAGATC | |
| | T | |
| Phenylketonuria; | ggccaaagcatgcatGAGGACAGTACTCACGGTTCGTTTCAGAGCTA | 559 |
| not | TGCTGGAAACAGCATAGCAAGTTGAAATAAGGCTAGTCCGTTA | |
| provided | TCAACTTGAAAAAGTGGCACCGAGTCGGTGCCCCATGTATACC | |
| | CCCGAACCGTGAGTACTGTAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTAGTACATCAG | |
| | ACATGGATCCAAGCCCATGTATATCCCCGAACCGTGAGTACTG | |
| | TCCTCCAGCTACCAAACGGGgctaagcAGCTTGGCGTAACTAGA | |
| | TCT | |
| Phenylketonuria; | ggccaaagcatgcatGAGGACAGTACTCACGGTTCGTTTCAGAGCTA | 560 |
| not | TGCTGGAAACAGCATAGCAAGTTGAAATAAGGCTAGTCCGTTA | |
| provided | TCAACTTGAAAAAGTGGCACCGAGTCGGTGCCCAAGCCCATG | |
| | TATACCCCCGAACCGTGAGTACTGTAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTATCAG | |
| | ACATGGATCCAAGCCCATGTATATCCCCGAACCGTGAGTACTG | |
| | TCCTCCAGCTACCAGCCCCAgctaagcAGCTTGGCGTAACTAGAT | |
| | CT | |
| Phenylketonuria; | ggccaaagcatgcatGGGACAGTACTCACGGTTCGGTTTCAGAGCTA | 561 |
| not | TGCTGGAAACAGCATAGCAAGTTGAAATAAGGCTAGTCCGTTA | |
| provided | TCAACTTGAAAAAGTGGCACCGAGTCGGTGCATACCCCCGAA | |
| | CCGTGAGTACTGAAAAAAAAAGCTGCCATCAGTCGGCGTGGA | |
| | CTGTAGAACACTGCCAATGCCGGTCCCAAGCCCGGATAAAAG | |
| | TGGAGGGTACAGTCCACGCTTTTTTTACTGCACACAGTACATC | |
| | AGACATGGATCCAAGCCCATGTATATCCCCGAACCGTGAGTAC | |
| | TGTCCTCCAGCTACCAGTCTTgctaagcAGCTTGGCGTAACTAGA | |
| | TCT | |
| Phenylketonuria; | ggccaaagcatgcatGGGACAGTACTCACGGTTCGGTTTCAGAGCTA | 562 |
| not | TGCTGGAAACAGCATAGCAAGTTGAAATAAGGCTAGTCCGTTA | |
| provided | TCAACTTGAAAAAGTGGCACCGAGTCGGTGCCCCATGTATACC | |
| | CCCGAACCGTGAGTACTGAAAAAAAAAGCTGCCATCAGTCGG | |
| | CGTGGACTGTAGAACACTGCCAATGCCGGTCCCAAGCCCGGA | |
| | TAAAAGTGGAGGGTACAGTCCACGCTTTTTTTACAGTACATCA | |
| | GACATGGATCCAAGCCCATGTATATCCCCGAACCGTGAGTACT | |
| | GTCCTCCAGCTACCTCAACGgctaagcAGCTTGGCGTAACTAGAT | |
| | CT | |
| Phenylketonuria; | ggccaaagcatgcatGGGACAGTACTCACGGTTCGGTTTCAGAGCTA | 563 |
| not | TGCTGGAAACAGCATAGCAAGTTGAAATAAGGCTAGTCCGTTA | |
| provided | TCAACTTGAAAAAGTGGCACCGAGTCGGTGCCCAAGCCCATG | |
| | TATACCCCCGAACCGTGAGTACTGAAAAAAAAAGCTGCCATCA | |
| | GTCGGCGTGGACTGTAGAACACTGCCAATGCCGGTCCCAAGC | |
| | CCGGATAAAAGTGGAGGGTACAGTCCACGCTTTTTTTACATCA | |
| | GACATGGATCCAAGCCCATGTATATCCCCGAACCGTGAGTACT | |
| | GTCCTCCAGCTACCCCCCACgctaagcAGCTTGGCGTAACTAGA | |
| | TCT | |
| Phenylketonuria; | ggccaaagcatgcatGCACGGTTCGGGGGTATACAGTTTCAGAGCT | 564 |
| not | ATGCTGGAAACAGCATAGCAAGTTGAAATAAGGCTAGTCCGTT | |
| provided | ATCAACTTGAAAAAGTGGCACCGAGTCGGTGCAAGCCCATGT | |
| | ATACCCCCGAACCAAAAAAAAAGCTGCCATCAGTCGGCGTGG | |
| | ACTGTAGAACACTGCCAATGCCGGTCCCAAGCCCGGATAAAA | |
| | GTGGAGGGTACAGTCCACGCTTTTTTTCGAGTCTTCCACTGCA | |
| | CACAGTACATCAGACATGGATCCAAGTCCATGTATACCCCCGA | |
| | ACCGTGAGTACTGTCCCTCTCAgctaagcAGCTTGGCGTAACTAG | |
| | ATCT | |
| Phenylketonuria; | ggccaaagcatgcatGCACGGTTCGGGGGTATACAGTTTCAGAGCT | 565 |
| not | ATGCTGGAAACAGCATAGCAAGTTGAAATAAGGCTAGTCCGTT | |
| provided | ATCAACTTGAAAAAGTGGCACCGAGTCGGTGCATCCAAGCCC | |
| | ATGTATACCCCCGAACCAAAAAAAAAGCTGCCATCAGTCGGCG | |
| | TGGACTGTAGAACACTGCCAATGCCGGTCCCAAGCCCGGATA | |
| | AAAGTGGAGGGTACAGTCCACGCTTTTTTTTCTTCCACTGCAC | |
| | ACAGTACATCAGACATGGATCCAAGTCCATGTATACCCCCGAA | |
| | CCGTGAGTACTGTCCATATACgctaagcAGCTTGGCGTAACTAGA | |
| | TCT | |
| Phenylketonuria; | ggccaaagcatgcatGCACGGTTCGGGGGTATACAGTTTCAGAGCT | 566 |
| not | ATGCTGGAAACAGCATAGCAAGTTGAAATAAGGCTAGTCCGTT | |
| provided | ATCAACTTGAAAAAGTGGCACCGAGTCGGTGCTGGATCCAAG | |
| | CCCATGTATACCCCCGAACCAAAAAAAAAGCTGCCATCAGTCG | |
| | GCGTGGACTGTAGAACACTGCCAATGCCGGTCCCAAGCCCGG | |
| | ATAAAAGTGGAGGGTACAGTCCACGCTTTTTTTTCCACTGCAC | |
| | ACAGTACATCAGACATGGATCCAAGTCCATGTATACCCCCGAA | |
| | CCGTGAGTACTGTCCGTCTTCgctaagcAGCTTGGCGTAACTAGA | |
| | TCT | |
| Phenylketonuria; | ggccaaagcatgcatGCACGGTTCGGGGGTATACAGTTTCAGAGCT | 567 |
| not | ATGCTGGAAACAGCATAGCAAGTTGAAATAAGGCTAGTCCGTT | |
| provided | ATCAACTTGAAAAAGTGGCACCGAGTCGGTGCAGACATGGAT | |
| | CCAAGCCCATGTATACCCCCGAACCAAAAAAAAAGCTGCCATC | |
| | AGTCGGCGTGGACTGTAGAACACTGCCAATGCCGGTCCCAAG | |
| | CCCGGATAAAAGTGGAGGGTACAGTCCACGCTTTTTTTTGCAC | |
| | ACAGTACATCAGACATGGATCCAAGTCCATGTATACCCCCGAA | |
| | CCGTGAGTACTGTCCGTACGTgctaagcAGCTTGGCGTAACTAG | |
| | ATCT | |

SEQ ID NO: 568: Lentiviral plasmid (Lenti_Puro_U6_5′Ribo-Nsil_Blpl) that bears U6 promoter and 5′ Ribozyme domain for oligo pool for restriction cloning into Nsil and Blpl that can be used for stable expression of the FIG. 4 in human cell lines:

GAGGGCCTATTTCCCATGATTCCTTCATATTTGCATATACGATACAAGGCTGTTAGAGAGA
TAATTAGAATTAATTTGACTGTAAACACAAAGATATTAGTACAAAATACGTGACGTAGAAA
GTAATAATTTCTTGGGTAGTTTGCAGTTTTAAAATTATGTTTTAAAATGGACTATCATATGC
TTACCGTAACTTGAAAGTATTTCGATTTCTTGGCTTTATATATCTTGTGGAAAGGACGAAA
CACCGCCATCAGTCGCCGGTCCCAAGCCCGGATAAAATGGGAGGGGGGGGAAACCG
CCTAACCATGCCGACTGATGGCAGAAAAAAAAAAATGCATCGAAGGACTGGAGGTGAA
GGTTGACCAGAGCCAGTAGATCAGTGAGTCGAAAGGCAGGCTAAGCACGCGTGTACTA
GTGTCTCGAGCTTATTCCAGATGCGTGCGGATGGAATTCGAGCTCGGTACCATGCCAAA
AGCAAAGCGCTATCGCGCCTTACGTTACTGGCCGAAGCCGCTTGGAATAAGGCCGGTG
TGCGTTTGTCTATATGTTATTTTCCACCATATTGCCGTCTTTTGGCAATGTGAGGGCCCG
GAAACCTGGCCCTGTCTTCTTGACGAGCATTCCTAGGGGTCTTTCCCCTCTCGCCAAA
GGAATGCAAGGTCTGTTGAATGTCGTGAAGGAAGCAGTTCCTCTGGAAGCTTCTTGAA
GACAAACAACGTCTGTAGCGACCCTTTGCAGGCAGCGGAACCCCCCACCTGGCGACA
GGTGCCTCTGCGGCCAAAAGCCACGTGTATAAGATACACCTGCAAAGGCGGCACAACC
CCAGTGCCACGTTGTGAGTTGGATAGTTGTGGAAAGAGTCAAATGGCTCCCCTCAAGC
GTATTCAACAAGGGGCTGAAGGATGCCCAGAAGGTACCCCATTGTATGGGATCTGATCT
GGGGCCTCGGTGCACATGCTTTTCATGTGTTTAGTCGAGGTTAAAAAACGTCTAGGCCC
CCCGAACCACGGGGACGTGGTTTTCCTTTGAAAAACACGATGATAATAACATGACCGAG
TACAAGCCCACGGTGCGCCTCGCCACCCGCGACGACGTCCCCAGGGCCGTACGCAC
CCTCGCCGCCGCGTTCGCCGACTACCCCGCCACGCGCCACACCGTCGATCCGGACC
GCCACATCGAGCGGGTCACCGAGCTGCAAGAACTCTTCCTCACGCGCGTCGGGCTCG
ACATCGGCAAGGTGTGGGTCGCGGACGACGGCGCCGCGGTGGCGGTCTGGACCACG
CCGGAGAGCGTCGAAGCGGGGGCGGTGTTCGCCGAGATCGGCCCGCGCATGGCCGA
GTTGAGCGGTTCCCGGCTGGCCGCGCAGCAACAGATGGAGGGCCTCCTGGCGCCGC
ACCGGCCCAAGGAGCCCGCGTGGTTCCTGGCCACCGTCGGCGTCTCGCCCGACCAC
CAGGGCAAGGGTCTGGGCAGCGCCGTCGTGCTCCCCGGAGTGGAGGCCGCCGAGC
GCGCCGGGGTGCCCGCCTTCCTGGAGACCTCCGCGCCCCGCAACCTCCCCTTCTACG
AGCGGCTCGGCTTCACCGTCACCGCCGACGTCGAGGTGCCCGAAGGACCGCGCACC
TGGTGCATGACCCGCAAGCCCGGTGCCTAGGCTAGCTTGACTGACTGAGTCGACAATC
AACCTTTTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGC
TTTCATTTTCTCCTCCTTGTATAAATCCTGGTTGCTGTCTCTTTATGAGGAGTTGTGGCC
CGTTGTCAGGCAACGTGGCGTGGTGTGCACTGTGTTTGCTGACGCAACCCCCACTGG
TTGGGGCATTGCCACCACCTGTCAGCTCCTTTCCGGGACTTTCGCTTTCCCCCTCCCT
ATTGCCACGGCGGAACTCATCGCCGCCTGCCTTGCCCGCTGCTGGACAGGGGCTCGG
CTGTTGGGCACTGACAATTCCGTGGTGTTGTCGGGGAAGCTGACGTCCTTTCCATGGC
TGCTCGCCTGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCTCT
TCCGCGTCTTCGCCTTCGCCCTCAGACGAGTCGGATCTCCCTTTGGGCCGCCTCCCC
GCCTGGAATTCGAGCTCGGTACCTTTAAGACCAATGACTTACAAGGCAGCTGTAGATCT
TAGCCACTTTTTAAAAGAAAAGGGGGGACTGGAAGGGCTAATTCACTCCCAACGAAGA
CAAGATCTGCTTTTTGCTTGTACTGGGTCTCTCTGGTTAGACCAGATCTGAGCCTGGGA
GCTCTCTGGCTAACTAGGGAACCCACTGCTTAAGCCTCAATAAAGCTTGCCTTGAGTGC
TTCAAGTAGTGTGTGCCCGTCTGTTGTGTGACTCTGGTAACTAGAGATCCCTCAGACCC
TTTTAGTCAGTGTGGAAAATCTCTAGCAGTCCTGGCCAACGTGAGCACCGTGCTGACC
TCCAAATATCGTTAAGCTGGAGCCTGGGAGCCGGCCTGGCCCTCCGCCCCCCCCACC
CCCGCAGCCCACCCCTGGTCTTTGAATAAAGTCTGAGTGAGTGGCCGACAGTGCCCGT
GGAGTTCTCGTGACCTGAGGTGCAGGGCCGGCGCTAGGGACACGTCCGTGCACGTG
CCGAGGCCCCCTGTGCAGCTGCAAGGGACAGGCCTAGCCCTGCAGGCCTAACTCCGC
CCATCCCGCCCCTAACTCCGCCCAGTTCCGCCCATTCTCCGCCTCATGGCTGACTAATT
TTTTTTATTTATGCAGAGGCCGAGGCCGCCTCGGCCTCTGAGCTATTCCAGAAGTAGTG
AGGACGCTTTTTTGGAGGCCGAGGCTTTTGCAAAGATCGAACAAGAGACAGGACCTGC
AGGTTAATTAAATTTAAATCATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGAAC
AGTATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAGCA
GATTACGCGCAGAAAAAAAGGATCTCAAGAAGATCCTTTGATCTTTTCTACGGGGTCTG
ACGCTCAGTGGAACGAAAACTCACGTTAAGGGATTTTGGTCATGAGATTATCAAAAAGG
ATCTTCACCTAGATCCTTTTAAATTAAAAATGAAGTTTTAAATCAATCTAAAGTATATATGAG
TAAACTTGGTCTGACAGTTACCAATGCTTAATCAGTGAGGCACCTATCTCAGCGATCTGT
CTATTTCGTTCATCCATAGTTGCATTTAAATGGCCGGCCTGGCGCGCCGTTTAAACCTAG
ATATTGATAGTCTGATCGGTCAACGTATAATCGAGTCCTAGCTTTTGCAAACATCTATCAA
GAGACAGGATCAGCAGGAGGCTTTCGCATGAGTATTCAACATTTCCGTGTCGCCCTTAT
TCCCTTTTTTGCGGCATTTTGCCTTCCTGTTTTTGCTCACCCAGAAACGCTGGTGAAAG
TAAAAGATGCTGAAGATCAGTTGGGTGCGCGAGTGGGTTACATCGAACTGGATCTCAAC
AGCGGTAAGATCCTTGAGAGTTTTCGCCCCGAAGAACGCTTTCCAATGATGAGCACTTT
TAAAGTTCTGCTATGTGGCGCGGTATTATCCCGTATTGACGCCGGGCAAGAGCAACTCG
GTCGCCGCATACACTATTCTCAGAATGACTTGGTTGAGTATTCACCAGTCACAGAAAAG
CATCTTACGGATGGCATGACAGTAAGAGAATTATGCAGTGCTGCCATAACCATGAGTGAT
AACACTGCGGCCAACTTACTTCTGACAACGATTGGAGGACCGAAGGAGCTAACCGCTT
TTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACCT
TGCGTAAACTATTAACTGGCGAACTACTTACTCTAGCTTCCCGGCAACAGTTGATAGACT
GGATGGAGGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATCATTGCAGCA
CTGGGGCCAGATGGTAAGCCCTCCCGTATCGTAGTTATCTACACGACGGGGAGTCAGG
CAACTATGGATGAACGAAATAGACAGATCGCTGAGATAGGTGCCTCACTGATTAAGCATT
GGTAACCGATTCTAGGTGCATTGGCGCAGAAAAAAATGCCTGATGCGACGCTGCGCGT
CTTATACTCCCACATATGCCAGATTCAGCAACGGATACGGCTTCCCCAACTTGCCCACTT
CCATACGTGTCCTCCTTACCAGAAATTTATCCTTAAGATCCCGAATCGTTTAAACGCGAT
CGCAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCA
ATAATGACGTATGTTCCCATAGTAACGCCAATAGGGACTTTCCATTGACGTCAATGGGTG
GAGTATTTACGGTAAACTGCCCACTTGGCAGTACATCAAGTGTATCATATGCCAAGTACG
CCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTG
ATGCGGTTTTGGCAGTACATCAATGGGCGTGGATAGCGGTTTGACTCACGGGGATTTC
CAAGTCTCCACCCCATTGACGTCAATGGGAGTTTGTTTTGGCACCAAAATCAACGGGAC
TTTCCAAAATGTCGTAACAACTCCGCCCCATTGACGCAAATGGGCGGTAGGCGTGTAC
GGTGGGAGGTCTATATAAGCAGAGCTCGTTTAGTGAACCGGGGTCTCTCTGGTTAGAC
CAGATCTGAGCCTGGGAGCTCTCTGGCTAACTAGGGAACCCACTGCTTAAGCCTCAATA
AAGCTTGCCTTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGCGCCCGAAC
AGGGACCTGAAAGCGAAAGGGAAACCAGAGCTCTCTCGACGCAGGACTCGGCTTGCT
GAAGCGCGCACGGCAAGAGGCGAGGGGCGGCGACTGGTGAGTACGCCAAAAATTTTG
ACTAGCGGAGGCTAGAAGGAGAGAGATGGGTGCGAGAGCGTCAGTATTAAGCGGGGG
AGAATTAGATCGCGATGGGAAAAAATTCGGTTAAGGCCAGGGGGAAAGAAAAAATATAA
ATTAAAACATATAGTATGGGCAAGCAGGGAGCTAGAACGATTCGCAGTTAATCCTGGCCT
GTTAGAAACATCAGAAGGCTGTAGACAAATACTGGGACAGCTACAACCATCCCTTCAGA
CAGGATCAGAAGAACTTAGATCATTATATAATACAGTAGCAACCCTCTATTGTGTGCATCA
AAGGATAGAGATAAAAGACACCAAGGAAGCTTTAGACAAGATAGAGGAAGAGCAAAACA
AAAGTAAGACCACCGCACAGCAAGCGGCCGCTGATCTTCAGACCTGGAGGAGGAGAT
ATGAGGGACAATTGGAGAAGTGAATTATATAAATATAAAGTAGTAAAAATTGAACCATTAG
GAGTAGCACCCACCAAGGCAAAGAGAAGAGTGGTGCAGAGAGAAAAAAGAGCAGTGG
GAATAGGAGCTTTGTTCCTTGGGTTCTTGGGAGCAGCAGGAAGCACTATGGGCGCAGC
CTCAATGACGCTGACGGTACAGGCCAGACAATTATTGTCTGGTATAGTGCAGCAGCAGA
ACAATTTGCTGAGGGCTATTGAGGCGCAACAGCATCTGTTGCAACTCACAGTCTGGGG
CATCAAGCAGCTCCAGGCAAGAATCCTGGCTGTGGAAAGATACCTAAAGGATCAACAG
CTCCTGGGGATTTGGGGTTGCTCTGGAAAACTCATTTGCACCACTGCTGTGCCTTGGA
ATGCTAGTTGGAGTAATAAATCTCTGGAACAGATTTGGAATCACACGACCTGGATGGAG
TGGGACAGAGAAATTAACAATTACACAAGCTTAATACACTCCTTAATTGAAGAATCGCAA
AACCAGCAAGAAAAGAATGAACAAGAATTATTGGAATTAGATAAATGGGCAAGTTTGTGG
AATTGGTTTAACATAACAAATTGGCTGTGGTATATAAAATTATTCATAATGATAGTAGGAGG
CTTGGTAGGTTTAAGAATAGTTTTTGCTGTACTTTCTATAGTGAATAGAGTTAGGCAGGG
ATATTCACCATTATCGTTTCAGACCCACCTCCCAACCCCGAGGGGACCCGACAGGCCC
GAAGGAATAGAAGAAGAAGGTGGAGAGAGAGACAGAGACAGATCCATTCGATTAGTGA
ACGGATCTCGACGGTATCGGTTAACTTTTAAAAGAAAAGGGGGGATTGGGGGGTACAG
TGCAGGGGAAAGAATAGTAGACATAATAGCAACAGACATACAAACTAAAGAATTACAAAA
ACAAATTACAAAAATTCAAAATTTTGGCTCCCGATCGTTGCGTTACACACACAATTACTG
CTGATCGAGTGTAGCCTTCGAATGAAAGACCCCACCTGTAGGTTTGGCAAGATAGCTGC
AGTAACGCCATTTTGCAAGGCATGGAAAAATACCAAACCAAGAATAGAGAAGTTCAGAT
CAAGGGCGGGTACATGAAAATAGCTAACGTTGGGCCAAACAGGATATCTGCGGTGAGC
AGTTTCGGCCCCGGCCCGGGGCCAAGAACAGATGGTCACCGCAGTTTCGGCCCCGG
CCCGAGGCCAAGAACAGATGGTCCCCAGATATGGCCCAACCCTCAGCAGTTTCTTAAG
ACCCATCAGATGTTTCCAGGCTCCCCCAAGGACCTGAAATGACCCTGCGCCTTATTTGA
ATTAACCAATCAGCCTGCTTCTCGCTTCTGTTCGCGCGCTTCTGCTTCCCGAGCTCTAT
AAAAGAGCTCACAACCCCTCACTCGGCGCGCCAGTCCTCCGATTGACTGAGTCGCCCT
GATCATTGTCGATCCTACCATCCACTCGACACACCCGCCAGGGCCCGCATCCACCATC
GCAGACTTATCATGGATCC

TABLE 5
Circular (Rotated) pegRNA exemplary sequences.

| Name | Sequence | SEQ ID NO: |
|---|---|---|
| Circular roted pegRNA | gagggcctatttcccatgattccttcatatttgcatatacgatag | SEQ ID NO: 573 |
| HEK3_1TtoA | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCATCTCGTGCTCAGTCTGaaaaaaaaaaaaaaa | |
| | aaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 574 |
| HEK3_1TtoC | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCATCGCGTGCTCAGTCTGaaaaaaaaaaaaaa | |
| | aaaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 575 |
| FHEK3_1TtoG | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCATCCCGTGCTCAGTCTGaaaaaaaaaaaaaa | |
| | aaaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 576 |
| HEK3_2GtoA | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCATTACGTGCTCAGTCTGaaaaaaaaaaaaaaa | |
| | aaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 577 |
| HEK3_2GtoC | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCATGACGTGCTCAGTCTGaaaaaaaaaaaaaa | |
| | aaaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 578 |
| HEK3_2GtoT | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCATAACGTGCTCAGTCTGaaaaaaaaaaaaaaa | |
| | aaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 579 |
| HEK3_3AtoC | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCAGCACGTGCTCAGTCTGaaaaaaaaaaaaaa | |
| | aaaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 580 |
| HEK3_3AtoG | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCACCACGTGCTCAGTCTGaaaaaaaaaaaaaa | |
| | aaaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 581 |
| HEK3_3AtoT | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCAACACGTGCTCAGTCTGaaaaaaaaaaaaaa | |
| | aaaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 582 |
| HEK3_4TtoA | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaCGGCA | |
| | GCATAGCAAGTTAAAATAAGGCTAGTCCGTTATC | |
| | AACTTGAAAAAGTGGCACCGAGTCGGTGCTCT | |
| | GCCTTCACGTGCTCAGTCTGaaaaaaaaaaaaaaa | |
| | aaaaaGGCCCAGACTGAGCACGTGA | |
| | GTTTTAGAGCTATGCTGCCGaaaaaaaaagctgcca | |
| | tcagtcggcgtggactgtagaacactgccaatgccggtccca | |
| | agcccggataaaagtggagggtacagtccacgcTTTTTTAA | |
| | AGGACAGGATTAGGCCAGGAAGGAGGAAGCA | |
| | AGGAA | |
| Circular roted pegRNA | gagggcctatttcccatgattccttcatatttgcatatacgatag | SEQ ID NO: 583 |
| HEK3_1TtoA | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCATCTCGTGCTCAGTCTGa | |
| | aaaaaaaagctgccatcagtcggcgtggactgtagaacactg | |
| | ccaatgccggtcccaagcccggataaaagtggagggtacag | |
| | tccacgcTTTTTTAAAGGACAGGATTAGGCCAGGA | |
| | AGGAGGAAGCAAGGAA | |
| Circular pegRNA | gagggcctatttcccatgattccttcatatttgcatatacgatag | SEQ ID NO: 584 |
| HEK3_1TtoC | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCATCGCGTGCTCAGTCTG | |
| | aaaaaaaaagctgccatcagtcggcgtggactgtagaacac | |
| | tgccaatgccggtcccaagcccggataaaagtggagggtac | |
| | agtccacgcTTTTTTAAAGGACAGGATTAGGCCAG | |
| | GAAGGAGGAAGCAAGGAA | |
| Circular pegRNA | gagggcctatttcccatgattccttcatatttgcatatacgatag | SEQ ID NO: 585 |
| FHEK3_1TtoG | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCATCCCGTGCTCAGTCTG | |
| | aaaaaaaaagctgccatcagtcggcgtggactgtagaacac | |
| | tgccaatgccggtcccaagcccggataaaagtggagggtac | |
| | agtccacgcTTTTTTAAAGGACAGGATTAGGCCAG | |
| | GAAGGAGGAAGCAAGGAA | |
| Circular pegRNA | gagggcctatttcccatgattccttcatatttgcatatacgatag | SEQ ID NO: 586 |
| HEK3_2GtoA | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCATTACGTGCTCAGTCTGa | |
| | aaaaaaaagctgccatcagtcggcgtggactgtagaacactg | |
| | ccaatgccggtcccaagcccggataaaagtggagggtacag | |
| | tccacgcTTTTTTAAAGGACAGGATTAGGCCAGGA | |
| | AGGAGGAAGCAAGGAA | |
| Circular pegRNA | gagggcctatttcccatgattccttcatatttgcatatacgatag | SEQ ID NO: 587 |
| HEK3_2GtoC | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCATGACGTGCTCAGTCTG | |
| | aaaaaaaaagctgccatcagtcggcgtggactgtagaacac | |
| | tgccaatgccggtcccaagcccggataaaagtggagggtac | |
| | agtccacgcTTTTTTAAAGGACAGGATTAGGCCAG | |
| | GAAGGAGGAAGCAAGGAA | |
| Circular pegRNA | gagggcctatttcccatgattccttcatatttgcatatacgatag | SEQ ID NO: 588 |
| HEK3_2GtoT | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCATAACGTGCTCAGTCTGa | |
| | aaaaaaaagctgccatcagtcggcgtggactgtagaacactg | |
| | ccaatgccggtcccaagcccggataaaagtggagggtacag | |
| | tccacgcTTTTTTAAAGGACAGGATTAGGCCAGGA | |
| | AGGAGGAAGCAAGGAA | |
| Circular pegRNA | gagggcctatttcccatgattccttcatatttgcatatacgatag | SEQ ID NO: 589 |
| HEK3_3AtoC | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCAGCACGTGCTCAGTCTG | |
| | aaaaaaaaagctgccatcagtcggcgtggactgtagaacac | |
| | tgccaatgccggtcccaagcccggataaaagtggagggtac | |
| | agtccacgcTTTTTTAAAGGACAGGATTAGGCCAG | |
| | GAAGGAGGAAGCAAGGAA | |
| Circular pegRNA | gagggcctatttcccatgattccttcatatttgcatatacgatag | SEQ ID NO: 590 |
| HEK3_3AtoG | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCACCACGTGCTCAGTCTG | |
| | aaaaaaaaagctgccatcagtcggcgtggactgtagaacac | |
| | tgccaatgccggtcccaagcccggataaaagtggagggtac | |
| | agtccacgcTTTTTTAAAGGACAGGATTAGGCCAG | |
| | GAAGGAGGAAGCAAGGAA | |
| Circular pegRNA | gagggcctatttcccatgattccttcatatttgcatatacgatag | SEQ ID NO: 591 |
| HEK3_3AtoT | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCAACACGTGCTCAGTCTG | |
| | aaaaaaaaagctgccatcagtcggcgtggactgtagaacac | |
| | tgccaatgccggtcccaagcccggataaaagtggagggtac | |
| | agtccacgcTTTTTTAAAGGACAGGATTAGGCCAG | |
| | GAAGGAGGAAGCAAGGAA | |
| Circular pegRNA | gagggcctatttcccatgattccttcatatttgcatatacgatag | SEQ ID NO: 592 |
| HEK3_4TtoA | cttaccgtaacttgaaagtatttcgatttcttggctttatatatctt | |
| | gtggaaaggacgaaacaccgaaacaccgccatcagtcgcc | |
| | ggtcccaagcccggataaaatgggagggggcgggaaaccg | |
| | cctaaccatgccgactgatggcagaaaaaaaaaaGGCC | |
| | CAGACTGAGCACGTGA | |
| | GTTTTAGAGCTAGAAATAGCAAGTTAAAATAAGG | |
| | CTAGTCCGTTATCAACTTGAAAAAGTGGGACCG | |
| | AGTCGGTCCTCTGCCTTCACGTGCTCAGTCTGa | |
| | aaaaaaaagctgccatcagtcggcgtggactgtagaacactg | |
| | ccaatgccggtcccaagcccggataaaagtggagggtacag | |
| | tccacgcTTTTTTAAAGGACAGGATTAGGCCAGGA | |
| | AGGAGGAAGCAAGGAA | |
| Linear pegRNA | GGCCCAGACTGAGCACGTGA | SEQ ID NO: 593 |
| HEK3_1TtoA | GTTTTAGAGCTAGAAATAGCAAGTTAAAATAAGG | |
| | CTAGTCCGTTATCAACTTGAAAAAGTGGGACCG | |
| | AGTCGGTCCTCTGCCATCTCGTGCTCAGTCTGT | |
| | TTTTTAAAGGACAGGATTAGGCCAGGAAGGAGG | |
| | AAGCAAGGAA | |
| Linear pegRNA | GGCCCAGACTGAGCACGTGA | SEQ ID NO: 594 |
| HEK3_1TtoC | GTTTTAGAGCTAGAAATAGCAAGTTAAAATAAGG | |
| | CTAGTCCGTTATCAACTTGAAAAAGTGGGACCG | |
| | AGTCGGTCCTCTGCCATCGCGTGCTCAGTCTG | |
| | TTTTTTAAAGGACAGGATTAGGCCAGGAAGGAG | |
| | GAAGCAAGGAA | |
| Linear pegRNA | GGCCCAGACTGAGCACGTGA | SEQ ID NO: 595 |
| FHEK3_1TtoG | GTTTTAGAGCTAGAAATAGCAAGTTAAAATAAGG | |
| | CTAGTCCGTTATCAACTTGAAAAAGTGGGACCG | |
| | AGTCGGTCCTCTGCCATCCCGTGCTCAGTCTG | |
| | TTTTTTAAAGGACAGGATTAGGCCAGGAAGGAG | |
| | GAAGCAAGGAA | |
| Linear pegRNA | GGCCCAGACTGAGCACGTGA | SEQ ID NO: 596 |
| HEK3_2GtoA | GTTTTAGAGCTAGAAATAGCAAGTTAAAATAAGG | |
| | CTAGTCCGTTATCAACTTGAAAAAGTGGGACCG | |
| | AGTCGGTCCTCTGCCATTACGTGCTCAGTCTGT | |
| | TTTTTAAAGGACAGGATTAGGCCAGGAAGGAGG | |
| | AAGCAAGGAA | |
| Linear pegRNA | GGCCCAGACTGAGCACGTGA | SEQ ID NO: 597 |
| HEK3_2GtoC | GTTTTAGAGCTAGAAATAGCAAGTTAAAATAAGG | |
| | CTAGTCCGTTATCAACTTGAAAAAGTGGGACCG | |
| | AGTCGGTCCTCTGCCATGACGTGCTCAGTCTGT | |
| | TTTTTAAAGGACAGGATTAGGCCAGGAAGGAGG | |
| | AAGCAAGGAA | |
| Linear pegRNA | GGCCCAGACTGAGCACGTGA | SEQ ID NO: 598 |
| HEK3_2GtoT | GTTTTAGAGCTAGAAATAGCAAGTTAAAATAAGG | |
| | CTAGTCCGTTATCAACTTGAAAAAGTGGGACCG | |
| | AGTCGGTCCTCTGCCATAACGTGCTCAGTCTGT | |
| | TTTTTAAAGGACAGGATTAGGCCAGGAAGGAGG | |
| | AAGCAAGGAA | |
| Linear pegRNA | GGCCCAGACTGAGCACGTGA | SEQ ID NO: 599 |
| HEK3_3AtoC | GTTTTAGAGCTAGAAATAGCAAGTTAAAATAAGG | |
| | CTAGTCCGTTATCAACTTGAAAAAGTGGGACCG | |
| | AGTCGGTCCTCTGCCAGCACGTGCTCAGTCTG | |
| | TTTTTTAAAGGACAGGATTAGGCCAGGAAGGAG | |
| | GAAGCAAGGAA | |
| Linear pegRNA | GGCCCAGACTGAGCACGTGA | SEQ ID NO: 600 |
| HEK3_3AtoG | GTTTTAGAGCTAGAAATAGCAAGTTAAAATAAGG | |
| | CTAGTCCGTTATCAACTTGAAAAAGTGGGACCG | |
| | AGTCGGTCCTCTGCCACCACGTGCTCAGTCTG | |
| | TTTTTTAAAGGACAGGATTAGGCCAGGAAGGAG | |
| | GAAGCAAGGAA | |
| Linear pegRNA | GGCCCAGACTGAGCACGTGA | SEQ ID NO: 601 |
| HEK3_3AtoT | GTTTTAGAGCTAGAAATAGCAAGTTAAAATAAGG | |
| | CTAGTCCGTTATCAACTTGAAAAAGTGGGACCG | |
| | AGTCGGTCCTCTGCCAACACGTGCTCAGTCTG | |
| | TTTTTTAAAGGACAGGATTAGGCCAGGAAGGAG | |
| | GAAGCAAGGAA | |
| Linear pegRNA | GGCCCAGACTGAGCACGTGA | SEQ ID NO: 602 |
| HEK3_4TtoA | GTTTTAGAGCTAGAAATAGCAAGTTAAAATAAGG | |
| | CTAGTCCGTTATCAACTTGAAAAAGTGGGACCG | |
| | AGTCGGTCCTCTGCCTTCACGTGCTCAGTCTGT | |
| | TTTTTAAAGGACAGGATTAGGCCAGGAAGGAGG | |
| | AAGCAAGGAA | |
| Nick_sgRNA HEK3_4a_+90 | GTCAACCAGTATCCCGGTGCGTTTTAGAGCTAG | SEQ ID NO: 603 |
| | AAATAGCAAGTTAAAATAAGGCTAGTCCGTTATC | |
| | AACTTGAAAAAGTGGCACCGAGTCGGTGCTTTT | |
| | TTAAAGGACAGGATTAGGCCAGGAAGGAGGAA | |
| | GCAAGGAA | |

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A prime editing guide RNA (pegRNA) comprising:
a. a spacer domain comprising a sequence substantially complementary to a region of a first strand (non-edit strand) of a double-stranded target nucleic acid;
b. a gRNA core domain capable of associating with a nucleic acid programmable DNA binding protein (napDNAbp);
c. a nucleic acid synthesis template domain (RTT) comprising an edit template domain comprising a sequence having one or more nucleotide changes compared to a second strand (edit strand) of the double-stranded target nucleic acid, and optionally the nucleic acid synthesis template domain further comprises an homology arm domain comprising a sequence substantially complementary the second strand of the double-stranded target nucleic acid; and
d. a primer binding site (PBS) comprising a sequence substantially complementary to a region upstream of the region complementary to the nucleic acid synthesis template domain in the second strand of the double-stranded target nucleic acid, and
wherein:
(i) the pegRNA is circularized; or
(ii) the pegRNA comprises a first portion of the gRNA core domain at one of the 5′-end or the 3′-end, and a second portion of the gRNA core domain at the other of the 5′-end or the 3′-end, and wherein the first and second portions together form the gRNA core domain; or
(iii) the pegRNA comprises a first ribozyme and a first ligation sequence positioned 3′ to the first ribozyme at 5′-end, and a second ribozyme and a second ligation sequence positioned 3′ to the second ribozyme at the 3′-end, and wherein a portion of the first ligation sequence is complementary to a portion of the first ribozyme and a portion of the second ligation sequence is complementary to a portion of the second ribozyme, wherein a portion of the first ligation sequence is complementary to a portion of the second ligation sequence; and wherein the portion of the first ligation sequence complementary to the portion of the first ribozyme is complementary to the portion of the second ligation sequence complementary to the portion of the second ribozyme.

2. The pegRNA of claim 1, wherein the pegRNA is circularized.

3. The pegRNA of claim 1, wherein: the spacer domain is 5′ of the gRNA core domain, the gRNA core domain is 5′ of the nucleic acid synthesis template domain, and the nucleic acid synthesis template domain is 5′ the primer binding site.

4. The pegRNA of claim 1, wherein: a first portion of the gRNA core domain is 5′ of the nucleic acid synthesis template domain, the nucleic acid synthesis template domain is 5′ of the primer binding site, the primer binding site is 5′ of the spacer domain, and the spacer domain is 5′ of a second portion of the gRNA core domain, and wherein the first and second portions together form the gRNA core domain.

5. The pegRNA of claim 1, wherein: a first ligation sequence is 5′ of a portion of the gRNA core domain, the first portion of the gRNA core domain is 5′ of the nucleic acid synthesis template domain, the nucleic acid synthesis template domain is 5′ of the primer binding site, the primer binding site is 5′ of the spacer domain, the spacer domain is 5′ of the second portion of the gRNA core domain, and a second portion of the gRNA core domain is 5′ of a second ligation sequence, and wherein the first and second portions together form the gRNA core domain, and optionally, a portion of the first ligation sequence is complementary to a portion of the second ligation sequence.

6. The pegRNA of claim 1, wherein the pegRNA is a RNA:DNA chimera.

7. The pegRNA of claim 1, wherein nucleic acid synthesis template domain is a template for an RNA-dependent polymerase or a DNA-dependent polymerase.

8. The pegRNA of claim 1, wherein the one or more nucleotide changes comprises insertions of one or more nucleotides, substitutions of one or more nucleotides, deletions of one or more nucleotides, or a combination of any such nucleotide changes, as compared to the double-stranded target DNA sequence.

9. The pegRNA of claim 1, wherein: (i) the one or more nucleotide changes comprises a transition selected from the group consisting of: (a) T to C; (b) A to G; (c) C to T; (d) G to A; and (e) A to I; or (ii) the one or more nucleotide changes comprises a transversion selected from the group consisting of: (a) T to A; (b) T to G; (c) C to G; (d) C to A; (e) A to T; (f) A to C; (g) G to C; (h) G to T; (i) and A to I; or (iii) one or more nucleotide changes comprises changing (1) a G:C basepair to a T:A basepair, (2) a G:C basepair to an A:T basepair, (3) a G:C basepair to C:G basepair, (4) a T:A basepair to a G:C basepair, (5) a T:A basepair to an A:T basepair, (6) a T:A basepair to a C:G basepair, (7) a C:G basepair to a G:C basepair, (8) a C:G basepair to a T:A basepair, (9) a C:G basepair to an A:T basepair, (10) an A:T basepair to a T:A basepair, (11) an A:T basepair to a G:C basepair, or (12) an A:T basepair to a C:G basepair; or (iv) the one or more nucleotide changes comprises insertion of at least 1 nucleotide; or (v) the one or more nucleotide changes comprises deletion of at least nucleotide.

10. The pegRNA of claim 1, wherein a part of the nucleic acid synthesis template domain comprises a sequence substantially complementary to a region downstream of a nick region in a second strand of the double-stranded target nucleic acid.

11. The pegRNA of claim 1, wherein the spacer domain comprises a sequence having 100% complementarity to the first strand of the double-stranded target nucleic acid, or the spacer domain comprises a sequence having one or more (e.g., 1, 2, 3, 4, or 5) mismatches with the first strand of the double-stranded target nucleic acid.

12. The pegRNA of any one of claims 1-35, wherein the gRNA core domain comprises a nucleotide sequence having at least 80% identity to a sequence selected from the group consisting of:
(SEQ ID NO: 1)
GTTTCAGAGCTATGCTGGAAACAGCATAGCAAGTTGAAATAAGGCTAGT
CCGTTATCAACTTGAAAAAGTGGCACCGAGTCGGTGC;
and
(SEQ ID NO: 572)
GTTTTAGAGCTAGAAATAGCAAGTTAAAATAAGGCTAGTCCGTTATCAA
CTTGAAAAAGTGGGACCGAGTCGGTCC.

13. The pegRNA of claim 1, wherein the pegRNA does not comprise an RNA-binding protein recruitment domain.

14. The pegRNA of claim 1, wherein the nucleic acid programmable DNA binding protein is an RNA guided DNA-binding protein, optionally the nucleic acid programmable DNA binding protein is a CRISPR Cas enzyme, an Argonaute protein, an obligate mobile element guided activity (OMEGA) enzyme, a RuVC nucleases, or a homolog, ortholog or variant thereof.

15. The pegRNA of claim 1, wherein the nucleic acid modifying enzyme is a polymerase, an RNA deaminase, an RNA methylase, an RNA demethylase, a retrotransposon or an integrase fused with a polymerase.

16. A prime editing system, comprising: (a) a pegRNA of claim 1 or a nucleic acid encoding same; (b) a nucleic acid programmable DNA binding protein (napDNAbp); and (c) a nucleic acid modifying enzyme or a nucleic acid encoding same.

17. A composition comprising a pegRNA of claim 1 or a nucleic acid encoding same, optionally, the composition further comprising a nucleic acid programmable DNA binding protein or a nucleic acid encoding same, and/or a nucleic acid modifying enzyme or a nucleic acid encoding same.

18. A cell comprising a pegRNA of claim 1.

19. The cell of claim 18, wherein the cell is a mismatch repair (MMR) competent cell.

20. A method of introducing one or more changes in the nucleotide sequence of a target nucleic acid, the method comprising contacting a double-stranded target nucleic acid with a prime editing system of claim 16.
